(19) United States
(12) Patent Application Publication
(10) Pub. No.: US 2013/0067618 A1
(43) Pub. Date: Mar. 14, 2013

(54) METHODS AND COMPOSITIONS FOR WEED CONTROL

(71) Applicants: DANIEL ADER, St. Louis, MO (US); John J. Finnessy, Des Peres, MO (US); Zhaolong Li, St. Charles, MO (US); Christina Marie Taylor, Chesterfield, MO (US); Jennifer Chou Taylor, Ballwin, MO (US); Ronak Hasmukh Shah, Chesterfield, MO (US); Nengbing Tao, O'Fallon, MO (US); Dafu Wang, Chesterfield, MO (US); Lisa Marie Weaver, O'Fallon, MO (US)

(72) Inventors: DANIEL ADER, St. Louis, MO (US); John J. Finnessy, Des Peres, MO (US); Zhaolong Li, St. Charles, MO (US); Christina Marie Taylor, Chesterfield, MO (US); Jennifer Chou Taylor, Ballwin, MO (US); Ronak Hasmukh Shah, Chesterfield, MO (US); Nengbing Tao, O'Fallon, MO (US); Dafu Wang, Chesterfield, MO (US); Lisa Marie Weaver, O'Fallon, MO (US)

(21) Appl. No.: 13/612,929

(22) Filed: Sep. 13, 2012

Related U.S. Application Data
(60) Provisional application No. 61/534,061, filed on Sep. 13, 2011.

(51) Int. Cl. A01N 57/16; C12N 15/63; A01P 13/00; A01H 3/04; A01P 21/00; C12P 19/34; C07H 21/04

(52) U.S. Cl. 800/278; 435/91.1; 435/320.1; 504/128; 504/196; 536/24.5

(57) ABSTRACT

The present invention provides novel compositions for use to enhance weed control. Specifically, the present invention provides for methods and compositions that modulate acetolactate synthase in weed species. The present invention also provides for combinations of compositions and methods that enhance weed control.

This application claims benefit under 35USC §119(e) of U.S. provisional application Ser. No. 61/534,061 filed Sep. 13, 2011, herein incorporated by reference in it's entirety. The sequence listing that is contained in the file named “40_{—}21(58635)B seq listing.txt”, which is 1,997,877 bytes (measured in operating system MS-Windows) and was created on 9 Sep. 2012, is filed herewith and incorporated herein by reference.

### FIELD

The invention relates generally to the field of weed management. More specifically, the invention relates to acetolactate synthase genes in weedy plants and compositions containing polynucleotide molecules for modulating their expression. The invention further provides methods and compositions useful for weed control.

### BACKGROUND

Weeds are plants that compete with cultivated plants in an agronomic environment and cost farmers billions of dollars annually in crop losses and the expense of efforts to keep weeds under control. Weeds also serve as hosts for crop diseases and insect pests. The losses caused by weeds in agricultural production environments include decreases in crop yield, reduced crop quality, increased irrigation costs, increased harvesting costs, reduced land value, injury to livestock, and crop damage from insects and diseases harbored by the weeds. The principal means by which weeds cause these effects are: 1) competing with crop plants for water, nutrients, sunlight and other essentials for growth and development, 2) production of toxic or irritant chemicals that cause human or animal health problem, 3) production of immense quantities of seed or vegetative reproductive parts or both that contaminate agricultural products and perpetuate the species in agricultural lands, and 4) production on agricultural and nonagricultural lands of vast amounts of vegetation that must be disposed of. Herbicide tolerant weeds are a problem with nearly all herbicides in use, there is a need to effectively manage these weeds. There are over 365 weed biotypes currently identified as being herbicide resistant to one or more herbicides by the Herbicide Resistance Action Committee (HRAC), the North American Herbicide Resistance Action Committee (NAHRAC), and the Weed Science Society of America (WSSA).

The ALS (acetolactate synthase, also known as acetohydroxyacid synthase, AHAS) enzyme catalyzes the first step in the synthesis of the branched-chain amino acids (valine, leucine, and isoleucine). ALS includes a large subunit member and a small subunit member that function to provide regulated production of the branched-chain amino acids. This enzyme is the target of many ALS inhibiting herbicides that include members of the chemical families of Sulfonylureas, Imidazolinones, Triazolopyrimidines, Pyrimidinyl(thio)benzoates, and Sulfonylaminocarbonyl-triazolinones.

### SUMMARY

In one aspect, the invention comprises a method of weedy plant control comprising an external application to a weedy plant of a composition comprising a polynucleotide and a transfer agent, wherein the polynucleotide is essentially identical or essentially complementary to an ALS gene sequence, or to the RNA transcript of said ALS gene sequence, wherein said ALS gene sequence is selected from the group consisting of SEQ ID NO:1-45 and 1692-1788 or a polynucleotide fragment thereof, whereby the weedy plant growth or development or reproductive ability is reduced or the weedy plant is more sensitive to an ALS inhibitor herbicide relative to a weedy plant not treated with said composition. In this manner, plants that have become resistant to the application of glyphosate containing herbicides may be made more susceptible to the herbicidal effects of a glyphosate containing herbicide, thus potentiating the effect of the herbicide. The polynucleotide fragment is at least 18 contiguous nucleotides, at least 19 contiguous nucleotides, at least 20 contiguous nucleotides or at least 21 contiguous nucleotides in length and at least 85 percent identical to an ALS gene sequence selected from the group consisting of SEQ ID NO:1-45 and 1692-1788 and the transfer agent is an organosilicone composition or compound. The polynucleotide fragment can also be sense or anti-sense ssDNA or ssRNA, dsRNA, or dsDNA, or dsDNA/RNA hybrids. The composition can include more than one polynucleotide fragments, and the composition can include an ALS inhibitor herbicide and/or other herbicides that enhance the weed control activity of the composition.

In another aspect, polynucleotide molecules and methods for modulating ALS gene expression in weedy plant species are provided. The method reduces expression of an ALS gene in a weedy plant comprising an external application to a weedy plant of a composition comprising a polynucleotide and a transfer agent, wherein the polynucleotide is essentially identical or essentially complementary to an ALS gene sequence or fragment thereof, or to the RNA transcript of the ALS gene sequence or fragment thereof, wherein the ALS gene sequence is selected from the group consisting of SEQ ID NO:1-45 and 1692-1788 or a polynucleotide fragment thereof. The polynucleotide fragment fragment is at least 18 contiguous nucleotides, at least 19 contiguous nucleotides, at least 20 contiguous nucleotides at least 21 contiguous nucleotides in length and at least 85 percent identical to an ALS gene sequence selected from the group consisting of SEQ ID NO:1-45 and 1692-1788 and the transfer agent is an organosilicone compound. The polynucleotide fragment can also be sense or anti-sense ssDNA or ssRNA, dsRNA, or dsDNA, or dsDNA/RNA hybrids.

In a further aspect of the invention, the polynucleotide molecule containing composition of the invention may be combined with other herbicidal compounds to provide additional control of unwanted plants in a field of cultivated plants.

In a further aspect, the polynucleotide molecule composition may be combined with any one or more additional agricultural chemicals, such as, insecticides, fungicides, nematocides, bactericides, acaricides, growth regulators, chemosterilants, semiochemicals, repellents, attractants, pheromones, feeding stimulants, biopesticides, microbial pesticides or other biologically active compounds to form a multi-component pesticide giving an even broader spectrum of agricultural protection.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein. The invention can be more fully understood from the following description of the figures:

FIG. 1. Treatment of Amaranthus palmer plants with ssDNA trigger polynucleotides and ALS inhibitor herbicide (Staple®).

FIG. 2. Treatment of Amaranthus palmer plants with the ALS_pro_S1 trigger molecule demonstrating improved activity of the Staple® and Classic® herbicides.

### DETAILED DESCRIPTION

The invention provides a method and compositions containing a polynucleotide that provide for regulation of an ALS (acetolactate synthase) large subunit and ALS small subunit gene expression and enhanced control of weedy plant species and importantly ALS inhibitor resistant weed biotypes. Aspects of the method can be applied to manage various weedy plants in agronomic and other cultivated environments.

The following definitions and methods are provided to better define the present invention and to guide those of ordinary skill in the art in the practice of the present invention. Unless otherwise noted, terms are to be understood according to conventional usage by those of ordinary skill in the relevant art. Where a term is provided in the singular, the inventors also contemplate aspects of the invention described by the plural of that term.

By “non-transcribable” polynucleotides is meant that the polynucleotides do not comprise a complete polymerase II transcription unit.

As used herein “solution” refers to homogeneous mixtures and non-homogeneous mixtures such as suspensions, colloids, micelles, and emulsions.

Weedy plants are plants that compete with cultivated plants, those of particular importance include, but are not limited to important invasive and noxious weeds in crop production, such as, Amaranthus species—A. albus, A. blitoides, A. hybridus, A. palmeri, A. powellii, A. retroflexus, A. spinosus, A. tuberculatus, and A. viridis; Ambrosia species—A. trifida, A. artemisifolia; Lolium species—L. multiflorum, L. rigidium, L perenne; Digitaria species—D. insularis; Euphorbia species—E. heterophylla; Kochia species—K. scoparia; Sorghum species—S. halepense; Conyza species—C. bonariensis, C. canadensis, C. sumatrensis; Chloris species—C. truncate; Echinochola species—E. colona, E. crus-galli; Eleusine species—E. indica; Poa species—P. annua; Plantago species—P. lanceolata; Avena species—A. fatua; Chenopodium species—C. album; Setaria species—S. viridis, Abutilon theophrasti, Ipomoea species, Sesbania, species, Cassia species, Sida species, Brachiaria, species and Solanum species.

Additional weedy plant species found in cultivated areas include Alopecurus myosuroides, Avena sterilis, Avena sterilis ludoviciana, Brachiaria plantaginea, Bromus diandrus, Bromus rigidus, Cynosurus echinatus, Digitaria ciliaris, Digitaria ischaemum, Digitaria sanguinalis, Echinochloa oryzicola, Echinochloa phyllopogon, Eriochloa punctata, Hordeum glaucum, Hordeum leporinum, Ischaemum rugosum, Leptochloa chinensis, Lolium persicum, Phalaris minor, Phalaris paradoxa, Rottboellia exalta, Setaria faberi, Setaria viridis var, robusta-alba schreiber, Setaria viridis var, robusta-purpurea, Snowdenia polystachea, Sorghum sudanese, Alisma plantago-aquatica, Amaranthus lividus, Amaranthus quitensis, Ammania auriculata, Ammania coccinea, Anthemis cotula, Apera spica-venti, Bacopa rotundifolia, Bidens pilosa, Bidens subalternans, Brassica tournefortii, Bromus tectorum, Camelina microcarpa, Chrysanthemum coronarium, Cuscuta campestris, Cyperus difformis, Damasonium minus, Descurainia sophia, Diplotaxis tenuifolia, Echium plantagineum, Elatine triandra var, pedicellate, Euphorbia heterophylla, Fallopia convolvulus, Fimbristylis miliacea, Galeopsis tetrahit, Galium spurium, Helianthus annuus, Iva xanthifolia, Ixophorus unisetus, Ipomoea indica, Ipomoea purpurea, Ipomoea sepiaria, Ipomoea aquatic, Ipomoea triloba, Lactuca serriola, Limnocharis flava, Limnophila erecta, Limnophila sessiliflora, Lindernia dubia, Lindernia dubia var, major, Lindernia micrantha, Lindernia procumbens, Mesembryanthemum crystallinum, Monochoria korsakowii, Monochoria vaginalis, Neslia paniculata, Papaver rhoeas, Parthenium hysterophorus, Pentzia suffruticosa, Phalaris minor, Raphanus raphanistrum, Raphanus sativus, Rapistrum rugosum, Rotala indica var, uliginosa, Sagittaria guyanensis, Sagittaria montevidensis, Sagittaria pygmaea, Salsola iberica, Scirpus juncoides var, ohwianus, Scirpus mucronatus, Setaria lutescens, Sida spinosa, Sinapis arvensis, Sisymbrium orientate, Sisymbrium thellungii, Solanum ptycanthum, Sonchus asper, Sonchus oleraceus, Sorghum bicolor, Stellaria media, Thlaspi arvense, Xanthium strumarium, Arctotheca calendula, Conyza sumatrensis, Crassocephalum crepidiodes, Cuphea carthagenenis, Epilobium adenocaulon, Erigeron philadelphicus, Landoltia punctata, Lepidium virginicum, Monochoria korsakowii, Solanum americanum, Solanum nigrum, Vulpia bromoides, Youngia japonica, Hydrilla verticillata, Carduus nutans, Carduus pycnocephalus, Centaurea solstitialis, Cirsium arvense, Commelina diffusa, Convolvulus arvensis, Daucus carota, Digitaria ischaemum, Echinochloa crus-pavonis, Fimbristylis miliacea, Galeopsis tetrahit, Galium spurium, Limnophila erecta, Matricaria perforate, Papaver rhoeas, Ranunculus acris, Soliva sessilis, Sphenoclea zeylanica, Stellaria media, Nassella trichotoma, Stipa neesiana, Agrostis stolonifera, Polygonum aviculare, Alopecurus japonicus, Beckmannia syzigachne, Bromus tectorum, Chloris inflate, Echinochloa erecta, Portulaca oleracea, and Senecio vulgaris. It is believed that all plants contain a acetolactate synthase gene in their genome, the sequence of which can be isolated and polynucleotides made according to the methods of the present invention that are useful for regulation, suppressing or delaying the expression of the target ALS gene (either the large subunit or the small subunit or both) in the plants and the growth or development of the treated plants.

A cultivated plant may also be weedy plant when it occurs in unwanted environments. For example, corn plants growing in a soybean field. Transgenic crops with one or more herbicide tolerances will need specialized methods of management to control weeds and volunteer crop plants. The present invention enables the targeting of a transgene for herbicide tolerance to permit the treated plants to become sensitive to the herbicide. For example, transgene ALS DNA sequences in transgenic events that include DP-356043-5.

A “trigger” or “trigger polynucleotide” is a polynucleotide molecule that is homologous or complementary to a target gene polynucleotide. The trigger polynucleotide molecules modulate expression of the target gene when topically applied to a plant surface with a transfer agent, whereby a plant treated with said composition has its growth or development or reproductive ability regulated, suppressed or delayed or said plant is more sensitive to an ALS inhibitor herbicide as a result of said polynucleotide containing composition relative to a plant not treated with a composition containing the trigger molecule. Trigger polynucleotides disclosed herein are generally described in relation to the target gene sequence and maybe used in the sense (homologous) or antisense (complementary) orientation as single stranded molecules or comprise both strands as double stranded molecules or nucleotide variants and modified nucleotides thereof depending on the various regions of a gene being targeted.

It is contemplated that the composition of the present invention will contain multiple polynucleotides and herbicides that include but not limited to ALS gene trigger polynucleotides and an ALS inhibitor herbicide and anyone or more additional herbicide target gene trigger polynucleotides and the related herbicides and anyone or more additional essential gene trigger polynucleotides. Essential genes are genes in a plant that provide key enzymes or other proteins, for example, a biosynthetic enzyme, metabolizing enzyme, receptor, signal transduction protein, structural gene product, transcription factor, or transport protein; or regulating RNAs, such as, microRNAs, that are essential to the growth or survival of the organism or cell or involved in the normal growth and development of the plant (Meinke, et al., Trends Plant Sci. 2008 September; 13(9):483-91). The suppression of an essential gene enhances the effect of a herbicide that affects the function of a gene product different than the suppressed essential gene. The compositions of the present invention can include various trigger polynucleotides that modulate the expression of an essential gene other than ALS.

Herbicides for which transgenes for plant tolerance have been demonstrated and the method of the present invention can be applied, include but are not limited to: auxin-like herbicides, glyphosate, glufosinate, sulfonylureas, imidazolinones, bromoxynil, delapon, dicamba, cyclohezanedione, protoporphyrionogen oxidase inhibitors, 4-hydroxyphenyl-pyruvate-dioxygenase inhibitors herbicides. For example, transgenes and their polynucleotide molecules that encode proteins involved in herbicide tolerance are known in the art, and include, but are not limited to an 5-enolpyruvylshikimate-3-phosphate synthase (EPSPS), for example, as more fully described in U.S. Pat. Nos. 7,807,791 (SEQ ID NO:5); 6,248,876 B1; 5,627,061; 5,804,425; 5,633,435; 5,145,783; 4,971,908; 5,312,910; 5,188,642; 4,940,835; 5,866,775; 6,225,114 B1; 6,130,366; 5,310,667; 4,535,060; 4,769,061; 5,633,448; 5,510,471; U.S. Pat. No. Re. 36,449; U.S. Pat. Nos. RE 37,287 E; and 5,491,288; tolerance to sulfonylurea and/or imidazolinone, for example, as described more fully in U.S. Pat. Nos. 5,605,011; 5,013,659; 5,141,870; 5,767,361; 5,731,180; 5,304,732; 4,761,373; 5,331,107; 5,928,937; and 5,378,824; and international publication WO 96/33270; tolerance to hydroxyphenylpyruvatedioxygenases inhibiting herbicides in plants are described in U.S. Pat. Nos. 6,245,968 B1; 6,268,549; and 6,069,115; and U.S. Pat. No. 7,312,379 SEQ ID NO:3; U.S. Pat. No. 7,935,869; U.S. Pat. No. 7,304,209, SEQ ID NO:1, 3, 5 and 15; aryloxyalkanoate dioxygenase polynucleotides, which confer tolerance to 2,4-D and other phenoxy auxin herbicides as well as to aryloxyphenoxypropionate herbicides as described, for example, in WO2005/107437; U.S. Pat. No. 7,838,733 SEQ ID NO:5) and dicamba-tolerance polynucleotides as described, for example, in Herman et al. (2005) J. Biol. Chem. 280: 24759-24767. Other examples of herbicide-tolerance traits include those conferred by polynucleotides encoding an exogenous phosphinothricin acetyltransferase, as described in U.S. Pat. Nos. 5,969,213; 5,489,520; 5,550,318; 5,874,265; 5,919,675; 5,561,236; 5,648,477; 5,646,024; 6,177,616; and 5,879,903. Plants containing an exogenous phosphinothricin acetyltransferase can exhibit improved tolerance to glufosinate herbicides, which inhibit the enzyme glutamine synthase. Additionally, herbicide-tolerance polynucleotides include those conferred by polynucleotides conferring altered protoporphyrinogen oxidase (protox) activity, as described in U.S. Pat. Nos. 6,288,306 B1; 6,282,837 B1; and 5,767,373; and WO 01/12825. Plants containing such polynucleotides can exhibit improved tolerance to any of a variety of herbicides which target the protox enzyme (also referred to as protox inhibitors). Polynucleotides encoding a glyphosate oxidoreductase and a glyphosate-N-acetyl transferase (GOX described in U.S. Pat. No. 5,463,175 and GAT described in U.S. Patent publication 20030083480, dicamba monooxygenase U.S. Patent publication 20030135879, all of which are incorporated herein by reference); a polynucleotide molecule encoding bromoxynil nitrilase (Bxn described in U.S. Pat. No. 4,810,648 for Bromoxynil tolerance, which is incorporated herein by reference); a polynucleotide molecule encoding phytoene desaturase (crtI) described in Misawa et al, (1993) Plant J. 4:833-840 and Misawa et al, (1994) Plant J. 6:481-489 for norflurazon tolerance; a polynucleotide molecule encoding acetohydroxyacid synthase (AHAS, aka ALS) described in Sathasiivan et al. (1990) Nucl. Acids Res. 18:2188-2193 for tolerance to sulfonylurea herbicides; and the bar gene described in DeBlock, et al. (1987) EMBO J. 6:2513-2519 for glufosinate and bialaphos tolerance. The transgenic coding regions and regulatory elements of the herbicide tolerance genes are targets in which polynucleotide triggers and herbicides can be included in the composition of the present invention.

The composition of the present invention include a component that is an ALS inhibitor herbicide which includes but are not limited to amidosulfuron, azimsulfuron, bensulfuron-methyl, chlorimuron-ethyl, chlorsulfuron, cinosulfuron, cyclosulfamuron, ethametsulfuron-methyl, ethoxysulfuron, flazasulfuron, flupyrsulfuron-methyl-Na, foramsulfuron, halosulfuron-methyl, imazosulfuron, iodosulfuron, metsulfuron-methyl, nicosulfuron, oxasulfuron, primisulfuron-methyl, prosulfuron, pyrazosulfuron-ethyl, rimsulfuron, sulfometuron-methyl, sulfosulfuron, thifensulfuron-methyl, triasulfuron, tribenuron-methyl, trifloxysulfuron, triflusulfuron-methyl, tritosulfuron, imazapic, imazamethabenz-methyl, imazamox, imazapyr, imazaquin, imazethapyr, cloransulam-methyl, diclosulam, florasulam, flumetsulam, metosulam, bispyribac-Na, pyribenzoxim, pyriftalid, pyrithiobac-Na, pyriminobac-methyl, flucarbazone-Na, and procarbazone-Na.

Numerous herbicides (herein referred to as co-herbicides) are available that can be added to the composition, for example, members of the herbicide families that include but are not limited to amide herbicides, aromatic acid herbicides, arsenical herbicides, benzothiazole herbicides, benzoylcyclohexanedione herbicides, benzofuranyl alkylsulfonate herbicides, carbamate herbicides, cyclohexene oxime herbicides, cyclopropylisoxazole herbicides, dicarboximide herbicides, dinitroaniline herbicides, dinitrophenol herbicides, diphenyl ether herbicides, dithiocarbamate herbicides, halogenated aliphatic herbicides, imidazolinone herbicides, inorganic herbicides, nitrile herbicides, organophosphorus herbicides, oxadiazolone herbicides, oxazole herbicides, phenoxy herbicides, phenylenediamine herbicides, pyrazole herbicides, pyridazine herbicides, pyridazinone herbicides, pyridine herbicides, pyrimidinediamine herbicides, pyrimidinyloxybenzylamine herbicides, quaternary ammonium herbicides, thiocarbamate herbicides, thiocarbonate herbicides, thiourea herbicides, triazine herbicides, triazinone herbicides, triazole herbicides, triazolone herbicides, triazolopyrimidine herbicides, uracil herbicides, and urea herbicides. In particular, the rates of use of the added herbicides can be reduced in compositions comprising the polynucleotides. Use rate reductions of the additional added herbicides can be 10-25 percent, 26-50 percent, 51-75 percent or more can be achieved that enhance the activity of the polynucleotides and herbicide composition and is contemplated. Representative herbicides of the families include but are not limited to acetochlor, acifluorfen, acifluorfen-sodium, aclonifen, acrolein, alachlor, alloxydim, allyl alcohol, ametryn, amicarbazone, amidosulfuron, aminopyralid, amitrole, ammonium sulfamate, anilofos, asulam, atraton, atrazine, azimsulfuron, BCPC, beflubutamid, benazolin, benfluralin, benfuresate, bensulfuron, bensulfuron-methyl, bensulide, bentazone, benzfendizone, benzobicyclon, benzofenap, bifenox, bilanafos, bispyribac, bispyribac-sodium, borax, bromacil, bromobutide, bromoxynil, butachlor, butafenacil, butamifos, butralin, butroxydim, butylate, cacodylic acid, calcium chlorate, cafenstrole, carbetamide, carfentrazone, carfentrazone-ethyl, CDEA, CEPC, chlorflurenol, chlorflurenol-methyl, chloridazon, chlorimuron, chlorimuron-ethyl, chloroacetic acid, chlorotoluron, chlorpropham, chlorsulfuron, chlorthal, chlorthal-dimethyl, cinidon-ethyl, cinmethylin, cinosulfuron, cisanilide, clethodim, clodinafop, clodinafop-propargyl, clomazone, clomeprop, clopyralid, cloransulam, cloransulam-methyl, CMA, 4-CPB, CPMF, 4-CPP, CPPC, cresol, cumyluron, cyanamide, cyanazine, cycloate, cyclosulfamuron, cycloxydim, cyhalofop, cyhalofop-butyl, 2,4-D, 3,4-DA, daimuron, dalapon, dazomet, 2,4-DB, 3,4-DB, 2,4-DEB, desmedipham, dicamba, dichlobenil, ortho-dichlorobenzene, para-dichlorobenzene, dichlorprop, dichlorprop-P, diclofop, diclofop-methyl, diclosulam, difenzoquat, difenzoquat metilsulfate, diflufenican, diflufenzopyr, dimefuron, dimepiperate, dimethachlor, dimethametryn, dimethenamid, dimethenamid-P, dimethipin, dimethylarsinic acid, dinitramine, dinoterb, diphenamid, diquat, diquat dibromide, dithiopyr, diuron, DNOC, 3,4-DP, DSMA, EBEP, endothal, EPTC, esprocarb, ethalfluralin, ethametsulfuron, ethametsulfuron-methyl, ethofumesate, ethoxyfen, ethoxysulfuron, etobenzanid, fenoxaprop-P, fenoxaprop-P-ethyl, fentrazamide, ferrous sulfate, flamprop-M, flazasulfuron, florasulam, fluazifop, fluazifop-butyl, fluazifop-P, fluazifop-P-butyl, flucarbazone, flucarbazone-sodium, flucetosulfuron, fluchloralin, flufenacet, flufenpyr, flufenpyr-ethyl, flumetsulam, flumiclorac, flumiclorac-pentyl, flumioxazin, fluometuron, fluoroglycofen, fluoroglycofen-ethyl, flupropanate, flupyrsulfuron, flupyrsulfuron-methyl-sodium, flurenol, fluridone, fluorochloridone, fluoroxypyr, flurtamone, fluthiacet, fluthiacet-methyl, fomesafen, foramsulfuron, fosamine, glufosinate, glufosinate-ammonium, glyphosate, halosulfuron, halosulfuron-methyl, haloxyfop, haloxyfop-P, HC-252, hexazinone, imazamethabenz, imazamethabenz-methyl, imazamox, imazapic, imazapyr, imazaquin, imazethapyr, imazosulfuron, indanofan, iodomethane, iodosulfuron, iodosulfuron-methyl-sodium, ioxynil, isoproturon, isouron, isoxaben, isoxachlortole, isoxaflutole, karbutilate, lactofen, lenacil, linuron, MAA, MAMA, MCPA, MCPA-thioethyl, MCPB, mecoprop, mecoprop-P, mefenacet, mefluidide, mesosulfuron, mesosulfuron-methyl, mesotrione, metam, metamifop, metamitron, metazachlor, methabenzthiazuron, methylarsonic acid, methyldymron, methyl isothiocyanate, metobenzuron, metolachlor, S-metolachlor, metosulam, metoxuron, metribuzin, metsulfuron, metsulfuron-methyl, MK-66, molinate, monolinuron, MSMA, naproanilide, napropamide, naptalam, neburon, nicosulfuron, nonanoic acid, norflurazon, oleic acid (fatty acids), orbencarb, orthosulfamuron, oryzalin, oxadiargyl, oxadiazon, oxasulfuron, oxaziclomefone, oxyfluorfen, paraquat, paraquat dichloride, pebulate, pendimethalin, penoxsulam, pentachlorophenol, pentanochlor, pentoxazone, pethoxamid, petrolium oils, phenmedipham, phenmedipham-ethyl, picloram, picolinafen, pinoxaden, piperophos, potassium arsenite, potassium azide, pretilachlor, primisulfuron, primisulfuron-methyl, prodiamine, profluazol, profoxydim, prometon, prometryn, propachlor, propanil, propaquizafop, propazine, propham, propisochlor, propoxycarbazone, propoxycarbazone-sodium, propyzamide, prosulfocarb, prosulfuron, pyraclonil, pyraflufen, pyraflufen-ethyl, pyrazolynate, pyrazosulfuron, pyrazosulfuron-ethyl, pyrazoxyfen, pyribenzoxim, pyributicarb, pyridafol, pyridate, pyriftalid, pyriminobac, pyriminobac-methyl, pyrimisulfan, pyrithiobac, pyrithiobac-sodium, quinclorac, quinmerac, quinoclamine, quizalofop, quizalofop-P, rimsulfuron, sethoxydim, siduron, simazine, simetryn, SMA, sodium arsenite, sodium azide, sodium chlorate, sulcotrione, sulfentrazone, sulfometuron, sulfometuron-methyl, sulfosate, sulfosulfuron, sulfuric acid, tar oils, 2,3,6-TBA, TCA, TCA-sodium, tebuthiuron, tepraloxydim, terbacil, terbumeton, terbuthylazine, terbutryn, thenylchlor, thiazopyr, thifensulfuron, thifensulfuron-methyl, thiobencarb, tiocarbazil, topramezone, tralkoxydim, tri-allate, triasulfuron, triaziflam, tribenuron, tribenuron-methyl, tricamba, triclopyr, trietazine, trifloxysulfuron, trifloxysulfuron-sodium, trifluralin, triflusulfuron, triflusulfuron-methyl, trihydroxytriazine, tritosulfuron, [3-[2-chloro-4-fluoro-5-(-methyl-6-trifluoromethyl-2,4-dioxo-,2,3,44-etrahydropyrimidin-3-yl)phenoxy]-2-pyridyloxy]acetic acid ethyl ester (CAS RN 353292-3-6), 4-[(4,5-dihydro-3-methoxy-4-methyl-5-oxo)-H-,2,4-triazol-1-ylcarbonyl-sulfamoyl]-5-methylthiophene-3-carboxylic acid (BAY636), BAY747 (CAS RN 33504-84-2), topramezone (CAS RN 2063-68-8), 4-hydroxy-3-[[2-[(2-methoxyethoxy)methyl]-6-(trifluoro-methyl)-3-pyridi-nyl]carbonyl]-bicyclo[3.2.]oct-3-en-2-one (CAS RN 35200-68-5), and 4-hydroxy-3-[[2-(3-methoxypropyl)-6-(difluoromethyl)-3-pyridinyl]carbon-yl]-bicyclo[3.2.]oct-3-en-2-one. Additionally, including herbicidal compounds of unspecified modes of action as described in CN101279950A, CN101279951A, DE10000600A1, DE10116399A1, DE102004054666A1, DE102005014638A1, DE102005014906A1, DE102007012168A1, DE102010042866A1, DE10204951A1, DE10234875A1, DE10234876A1, DE10256353A1, DE10256354A1, DE10256367A1, EP1157991A2, EP1238586A1, EP2147919A1, EP2160098A2, JP03968012B2, JP2001253874A, JP2002080454A, JP2002138075A, JP2002145707A, JP2002220389A, JP2003064059A, JP2003096059A, JP2004051628A, JP2004107228A, JP2005008583A, JP2005239675A, JP2005314407A, JP2006232824A, JP2006282552A, JP2007153847A, JP2007161701A, JP2007182404A, JP2008074840A, JP2008074841A, JP2008133207A, JP2008133218A, JP2008169121A, JP2009067739A, JP2009114128A, JP2009126792A, JP2009137851A, US20060111241A1, US20090036311A1, US20090054240A1, US20090215628A1, US20100099561A1, US20100152443A1, US20110105329A1, US20110201501A1, WO2001055066A2, WO2001056975A1, WO2001056979A1, WO2001090071A2, WO2001090080A1, WO2002002540A1, WO2002028182A1, WO2002040473A1, WO2002044173A2, WO2003000679A2, WO2003006422A1, WO2003013247A1, WO2003016308A1, WO2003020704A1, WO2003022051A1, WO2003022831A1, WO2003022843A1, WO2003029243A2, WO2003037085A1, WO2003037878A1, WO2003045878A2, WO2003050087A2, WO2003051823A1, WO2003051824A1, WO2003051846A2, WO2003076409A1, WO2003087067A1, WO2003090539A1, WO2003091217A1, WO2003093269A2, WO2003104206A2, WO2004002947A1, WO2004002981A2, WO2004011429A1, WO2004029060A1, WO2004035545A2, WO2004035563A1, WO2004035564A1, WO2004037787A1, WO2004067518A1, WO2004067527A1, WO2004077950A1, WO2005000824A1, WO2005007627A1, WO2005040152A1, WO2005047233A1, WO2005047281A1, WO2005061443A2, WO2005061464A1, WO2005068434A1, WO2005070889A1, WO2005089551A1, WO2005095335A1, WO2006006569A1, WO2006024820A1, WO2006029828A1, WO2006029829A1, WO2006037945A1, WO2006050803A1, WO2006090792A1, WO2006123088A2, WO2006125687A1, WO2006125688A1, WO2007003294A1, WO2007026834A1, WO2007071900A1, WO2007077201A1, WO2007077247A1, WO2007096576A1, WO2007119434A1, WO2007134984A1, WO2008009908A1, WO2008029084A1, WO2008059948A1, WO2008071918A1, WO2008074991A1, WO2008084073A1, WO2008100426A2, WO2008102908A1, WO2008152072A2, WO2008152073A2, WO2009000757A1, WO2009005297A2, WO2009035150A2, WO2009063180A1, WO2009068170A2, WO2009068171A2, WO2009086041A1, WO2009090401A2, WO2009090402A2, WO2009115788A1, WO2009116558A1, WO2009152995A1, WO2009158258A1, WO2010012649A1, WO2010012649A1, WO2010026989A1, WO2010034153A1, WO2010049270A1, WO2010049369A1, WO2010049405A1, WO2010049414A1, WO2010063422A1, WO2010069802A1, WO2010078906A2, WO2010078912A1, WO2010104217A1, WO2010108611A1, WO2010112826A3, WO2010116122A3, WO2010119906A1, WO2010130970A1, WO2011003776A2, WO2011035874A1, WO2011065451A1, all of which are incorporated herein by reference.

The trigger polynucleotide and oligonucleotide molecule compositions are useful in compositions, such as liquids that comprise the polynucleotide molecules at low concentrations, alone or in combination with other components, for example one or more herbicide molecules, either in the same solution or in separately applied liquids that also provide a transfer agent. While there is no upper limit on the concentrations and dosages of polynucleotide molecules that can useful in the methods, lower effective concentrations and dosages will generally be sought for efficiency. The concentrations can be adjusted in consideration of the volume of spray or treatment applied to plant leaves or other plant part surfaces, such as flower petals, stems, tubers, fruit, anthers, pollen, or seed. In one embodiment, a useful treatment for herbaceous plants using 25-mer oligonucleotide molecules is about 1 nanomole (nmol) of oligonucleotide molecules per plant, for example, from about 0.05 to 1 nmol per plant. Other embodiments for herbaceous plants include useful ranges of about 0.05 to about 100 nmol, or about 0.1 to about 20 nmol, or about 1 nmol to about 10 nmol of polynucleotides per plant. Very large plants, trees, or vines may require correspondingly larger amounts of polynucleotides. When using long dsRNA molecules that can be processed into multiple oligonucleotides, lower concentrations can be used. To illustrate embodiments, the factor 1×, when applied to oligonucleotide molecules is arbitrarily used to denote a treatment of 0.8 nmol of polynucleotide molecule per plant; 10×, 8 nmol of polynucleotide molecule per plant; and 100×, 80 nmol of polynucleotide molecule per plant.

The polynucleotide compositions are useful in compositions, such as liquids that comprise polynucleotide molecules, alone or in combination with other components either in the same liquid or in separately applied liquids that provide a transfer agent. As used herein, a transfer agent is an agent that, when combined with a polynucleotide in a composition that is topically applied to a target plant surface, enables the polynucleotide to enter a plant cell. In certain embodiments, a transfer agent is an agent that conditions the surface of plant tissue, e.g., leaves, stems, roots, flowers, or fruits, to permeation by the polynucleotide molecules into plant cells. The transfer of polynucleotides into plant cells can be facilitated by the prior or contemporaneous application of a polynucleotide-transferring agent to the plant tissue. In some embodiments the transferring agent is applied subsequent to the application of the polynucleotide composition. The polynucleotide transfer agent enables a pathway for polynucleotides through cuticle wax barriers, stomata and/or cell wall or membrane barriers into plant cells. Suitable transfer agents to facilitate transfer of the polynucleotide into a plant cell include agents that increase permeability of the exterior of the plant or that increase permeability of plant cells to oligonucleotides or polynucleotides. Such agents to facilitate transfer of the composition into a plant cell include a chemical agent, or a physical agent, or combinations thereof. Chemical agents for conditioning or transfer include (a) surfactants, (b) an organic solvent or an aqueous solution or aqueous mixtures of organic solvents, (c) oxidizing agents, (d) acids, (e) bases, (f) oils, (g) enzymes, or combinations thereof. Embodiments of the method can optionally include an incubation step, a neutralization step (e.g., to neutralize an acid, base, or oxidizing agent, or to inactivate an enzyme), a rinsing step, or combinations thereof. Embodiments of agents or treatments for conditioning of a plant to permeation by polynucleotides include emulsions, reverse emulsions, liposomes, and other micellar-like compositions. Embodiments of agents or treatments for conditioning of a plant to permeation by polynucleotides include counter-ions or other molecules that are known to associate with nucleic acid molecules, e.g., inorganic ammonium ions, alkyl ammonium ions, lithium ions, polyamines such as spermine, spermidine, or putrescine, and other cations. Organic solvents useful in conditioning a plant to permeation by polynucleotides include DMSO, DMF, pyridine, N-pyrrolidine, hexamethylphosphoramide, acetonitrile, dioxane, polypropylene glycol, other solvents miscible with water or that will dissolve phosphonucleotides in non-aqueous systems (such as is used in synthetic reactions). Naturally derived or synthetic oils with or without surfactants or emulsifiers can be used, e.g., plant-sourced oils, crop oils (such as those listed in the 9^{th} Compendium of Herbicide Adjuvants, publicly available on the worldwide web (internet) at herbicide.adjuvants.com can be used, e.g., paraffinic oils, polyol fatty acid esters, or oils with short-chain molecules modified with amides or polyamines such as polyethyleneimine or N-pyrrolidine. Transfer agents include, but are not limited to, organosilicone preparations.

An agronomic field in need of plant control is treated by application of the composition of the present invention directly to the surface of the growing plants, such as by a spray. For example, the method is applied to control weeds in a field of crop plants by spraying the field with the composition. The composition can be provided as a tank mix, a sequential treatment of components (generally the polynucleotide containing composition followed by the herbicide), or a simultaneous treatment or mixing of one or more of the components of the composition from separate containers. Treatment of the field can occur as often as needed to provide weed control and the components of the composition can be adjusted to target specific weed species or weed families through utilization of specific polynucleotides or polynucleotide compositions capable of selectively targeting the specific species or plant family to be controlled. The composition can be applied at effective use rates according to the time of application to the field, for example, preplant, at planting, post planting, post harvest. ALS inhibitor herbicides can be applied to a field at rates of 3 to 150 g ai/ha or more. The polynucleotides of the composition can be applied at rates of 1 to 30 grams per acre depending on the number of trigger molecules needed for the scope of weeds in the field.

Crop plants in which weed control is needed include but are not limited to, corn, soybean, cotton, canola, sugar beet, alfalfa, sugarcane, rice, and wheat; ii) vegetable plants including, but not limited to, tomato, sweet pepper, hot pepper, melon, watermelon, cucumber, eggplant, cauliflower, broccoli, lettuce, spinach, onion, peas, carrots, sweet corn, Chinese cabbage, leek, fennel, pumpkin, squash or gourd, radish, Brussels sprouts, tomatillo, garden beans, dry beans, or okra; iii) culinary plants including, but not limited to, basil, parsley, coffee, or tea; or, iv) fruit plants including but not limited to apple, pear, cherry, peach, plum, apricot, banana, plantain, table grape, wine grape, citrus, avocado, mango, or berry; v) a tree grown for ornamental or commercial use, including, but not limited to, a fruit or nut tree; or, vi) an ornamental plant (e.g., an ornamental flowering plant or shrub or turf grass). The methods and compositions provided herein can also be applied to plants produced by a cutting, cloning, or grafting process (i.e., a plant not grown from a seed) include fruit trees and plants that include, but are not limited to, citrus, apples, avocados, tomatoes, eggplant, cucumber, melons, watermelons, and grapes as well as various ornamental plants.

### Pesticidal Mixtures

The polynucleotide compositions may also be used as mixtures with various agricultural chemicals and/or insecticides, miticides and fungicides, pesticidal and biopesticidal agents. Examples include but are not limited to azinphos-methyl, acephate, isoxathion, isofenphos, ethion, etrimfos, oxydemeton-methyl, oxydeprofos, quinalphos, chlorpyrifos, chlorpyrifos-methyl, chlorfenvinphos, cyanophos, dioxabenzofos, dichlorvos, disulfoton, dimethylvinphos, dimethoate, sulprofos, diazinon, thiometon, tetrachlorvinphos, temephos, tebupirimfos, terbufos, naled, vamidothion, pyraclofos, pyridafenthion, pirimiphos-methyl, fenitrothion, fenthion, phenthoate, flupyrazophos, prothiofos, propaphos, profenofos, phoxime, phosalone, phosmet, formothion, phorate, malathion, mecarbam, mesulfenfos, methamidophos, methidathion, parathion, methyl parathion, monocrotophos, trichlorphon, EPN, isazophos, isamidofos, cadusafos, diamidaphos, dichlofenthion, thionazin, fenamiphos, fosthiazate, fosthietan, phosphocarb, DSP, ethoprophos, alanycarb, aldicarb, isoprocarb, ethiofencarb, carbaryl, carbosulfan, xylylcarb, thiodicarb, pirimicarb, fenobucarb, furathiocarb, propoxur, bendiocarb, benfuracarb, methomyl, metolcarb, XMC, carbofuran, aldoxycarb, oxamyl, acrinathrin, allethrin, esfenvalerate, empenthrin, cycloprothrin, cyhalothrin, gamma-cyhalothrin, lambda-cyhalothrin, cyfluthrin, beta-cyfluthrin, cypermethrin, alpha-cypermethrin, zeta-cypermethrin, silafluofen, tetramethrin, tefluthrin, deltamethrin, tralomethrin, bifenthrin, phenothrin, fenvalerate, fenpropathrin, furamethrin, prallethrin, flucythrinate, fluvalinate, flubrocythrinate, permethrin, resmethrin, ethofenprox, cartap, thiocyclam, bensultap, acetamiprid, imidacloprid, clothianidin, dinotefuran, thiacloprid, thiamethoxam, nitenpyram, chlorfluazuron, diflubenzuron, teflubenzuron, triflumuron, novaluron, noviflumuron, bistrifluoron, fluazuron, flucycloxuron, flufenoxuron, hexaflumuron, lufenuron, chromafenozide, tebufenozide, halofenozide, methoxyfenozide, diofenolan, cyromazine, pyriproxyfen, buprofezin, methoprene, hydroprene, kinoprene, triazamate, endosulfan, chlorfenson, chlorobenzilate, dicofol, bromopropylate, acetoprole, fipronil, ethiprole, pyrethrin, rotenone, nicotine sulphate, BT (Bacillus Thuringiensis) agent, spinosad, abamectin, acequinocyl, amidoflumet, amitraz, etoxazole, chinomethionat, clofentezine, fenbutatin oxide, dienochlor, cyhexatin, spirodiclofen, spiromesifen, tetradifon, tebufenpyrad, binapacryl, bifenazate, pyridaben, pyrimidifen, fenazaquin, fenothiocarb, fenpyroximate, fluacrypyrim, fluazinam, flufenzin, hexythiazox, propargite, benzomate, polynactin complex, milbemectin, lufenuron, mecarbam, methiocarb, mevinphos, halfenprox, azadirachtin, diafenthiuron, indoxacarb, emamectin benzoate, potassium oleate, sodium oleate, chlorfenapyr, tolfenpyrad, pymetrozine, fenoxycarb, hydramethylnon, hydroxy propyl starch, pyridalyl, flufenerim, flubendiamide, flonicamid, metaflumizole, lepimectin, TPIC, albendazole, oxibendazole, oxfendazole, trichlamide, fensulfothion, fenbendazole, levamisole hydrochloride, morantel tartrate, dazomet, metam-sodium, triadimefon, hexaconazole, propiconazole, ipconazole, prochloraz, triflumizole, tebuconazole, epoxiconazole, difenoconazole, flusilazole, triadimenol, cyproconazole, metconazole, fluquinconazole, bitertanol, tetraconazole, triticonazole, flutriafol, penconazole, diniconazole, fenbuconazole, bromuconazole, imibenconazole, simeconazole, myclobutanil, hymexazole, imazalil, furametpyr, thifluzamide, etridiazole, oxpoconazole, oxpoconazole fumarate, pefurazoate, prothioconazole, pyrifenox, fenarimol, nuarimol, bupirimate, mepanipyrim, cyprodinil, pyrimethanil, metalaxyl, mefenoxam, oxadixyl, benalaxyl, thiophanate, thiophanate-methyl, benomyl, carbendazim, fuberidazole, thiabendazole, manzeb, propineb, zineb, metiram, maneb, ziram, thiuram, chlorothalonil, ethaboxam, oxycarboxin, carboxin, flutolanil, silthiofam, mepronil, dimethomorph, fenpropidin, fenpropimorph, spiroxamine, tridemorph, dodemorph, flumorph, azoxystrobin, kresoxim-methyl, metominostrobin, orysastrobin, fluoxastrobin, trifloxystrobin, dimoxystrobin, pyraclostrobin, picoxystrobin, iprodione, procymidone, vinclozolin, chlozolinate, flusulfamide, dazomet, methyl isothiocyanate, chloropicrin, methasulfocarb, hydroxyisoxazole, potassium hydroxyisoxazole, echlomezol, D-D, carbam, basic copper chloride, basic copper sulfate, copper nonylphenolsulfonate, oxine copper, DBEDC, anhydrous copper sulfate, copper sulfate pentahydrate, cupric hydroxide, inorganic sulfur, wettable sulfur, lime sulfur, zinc sulfate, fentin, sodium hydrogen carbonate, potassium hydrogen carbonate, sodium hypochlorite, silver, edifenphos, tolclofos-methyl, fosetyl, iprobenfos, dinocap, pyrazophos, carpropamid, fthalide, tricyclazole, pyroquilon, diclocymet, fenoxanil, kasugamycin, validamycin, polyoxins, blasticiden S, oxytetracycline, mildiomycin, streptomycin, rape seed oil, machine oil, benthiavalicarbisopropyl, iprovalicarb, propamocarb, diethofencarb, fluoroimide, fludioxanil, fenpiclonil, quinoxyfen, oxolinic acid, chlorothalonil, captan, folpet, probenazole, acibenzolar-S-methyl, tiadinil, cyflufenamid, fenhexamid, diflumetorim, metrafenone, picobenzamide, proquinazid, famoxadone, cyazofamid, fenamidone, zoxamide, boscalid, cymoxanil, dithianon, fluazinam, dichlofluanide, triforine, isoprothiolane, ferimzone, diclomezine, tecloftalam, pencycuron, chinomethionat, iminoctadine acetate, iminoctadine albesilate, ambam, polycarbamate, thiadiazine, chloroneb, nickel dimethyldithiocarbamate, guazatine, dodecylguanidine-acetate, quintozene, tolylfluanid, anilazine, nitrothalisopropyl, fenitropan, dimethirimol, benthiazole, harpin protein, flumetover, mandipropamide and penthiopyrad.

### Polynucleotides

As used herein, the term “DNA”, “DNA molecule”, “DNA polynucleotide molecule” refers to a single-stranded DNA (ssDNA) or double-stranded DNA (dsDNA) molecule of genomic or synthetic origin, such as, a polymer of deoxyribonucleotide bases or a DNA polynucleotide molecule. As used herein, the term “DNA sequence”, “DNA nucleotide sequence” or “DNA polynucleotide sequence” refers to the nucleotide sequence of a DNA molecule. As used herein, the term “RNA”, “RNA molecule”, “RNA polynucleotide molecule” refers to a single-stranded RNA (ssRNA) or double-stranded RNA (dsRNA) molecule of genomic or synthetic origin, such as, a polymer of ribonucleotide bases that comprise single or double stranded regions. Unless otherwise stated, nucleotide sequences in the text of this specification are given, when read from left to right, in the 5′ to 3′ direction. The nomenclature used herein is that required by Title 37 of the United States Code of Federal Regulations §1.822 and set forth in the tables in WIPO Standard ST.25 (1998), Appendix 2, Tables 1 and 3.

As used herein, “polynucleotide” refers to a DNA or RNA molecule containing multiple nucleotides and generally refers both to “oligonucleotides” (a polynucleotide molecule of typically 50 or fewer nucleotides in length) and polynucleotides of 51 or more nucleotides. Embodiments of this invention include compositions including oligonucleotides having a length of 18-25 nucleotides (18-mers, 19-mers, 20-mers, 21-mers, 22-mers, 23-mers, 24-mers, or 25-mers), for example, oligonucleotides SEQ ID NO: 1364-1691 and 4167-4201 or fragments thereof, or medium-length polynucleotides having a length of 26 or more nucleotides (polynucleotides of 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, or about 300 nucleotides), for example, oligonucleotides SEQ ID NO: 46-1363 and 1789-4166 or fragments thereof or long polynucleotides having a length greater than about 300 nucleotides (for example, polynucleotides of between about 300 to about 400 nucleotides, between about 400 to about 500 nucleotides, between about 500 to about 600 nucleotides, between about 600 to about 700 nucleotides, between about 700 to about 800 nucleotides, between about 800 to about 900 nucleotides, between about 900 to about 1000 nucleotides, between about 300 to about 500 nucleotides, between about 300 to about 600 nucleotides, between about 300 to about 700 nucleotides, between about 300 to about 800 nucleotides, between about 300 to about 900 nucleotides, or about 1000 nucleotides in length, or even greater than about 1000 nucleotides in length, for example up to the entire length of a target gene including coding or non-coding or both coding and non-coding portions of the target gene) for example, polynucleotides of Table 1 (SEQ ID NO:1-45 and 1692-1788), wherein the selected polynucleotides or fragments thereof are homologous or complementary to SEQ ID NO:1-45 and 1692-1788 and suppresses, represses or otherwise delay the expression of the target ALS gene. A target gene comprises any polynucleotide molecule in a plant cell or fragment thereof for which the modulation of the expression of the target gene is provided by the methods and compositions of the present invention. Where a polynucleotide is double-stranded, its length can be similarly described in terms of base pairs. Oligonucleotides and polynucleotides of the present invention can be made that are essentially identical or essentially complementary to adjacent genetic elements of a gene, for example, spanning the junction region of an intron and exon, the junction region of a promoter and a transcribed region, the junction region of a 5′ leader and a coding sequence, the junction of a 3′ untranslated region and a coding sequence.

Polynucleotide compositions used in the various embodiments of this invention include compositions including oligonucleotides or polynucleotides or a mixture of both, including RNA or DNA or RNA/DNA hybrids or chemically modified oligonucleotides or polynucleotides or a mixture thereof. In some embodiments, the polynucleotide may be a combination of ribonucleotides and deoxyribonucleotides, for example, synthetic polynucleotides consisting mainly of ribonucleotides but with one or more terminal deoxyribonucleotides or synthetic polynucleotides consisting mainly of deoxyribonucleotides but with one or more terminal dideoxyribonucleotides. In some embodiments, the polynucleotide includes non-canonical nucleotides such as inosine, thiouridine, or pseudouridine. In some embodiments, the polynucleotide includes chemically modified nucleotides. Examples of chemically modified oligonucleotides or polynucleotides are well known in the art; see, for example, US Patent Publication 20110171287, US Patent Publication 20110171176, and US Patent Publication 20110152353, US Patent Publication, 20110152346, US Patent Publication 20110160082, herein incorporated by reference. For example, including but not limited to the naturally occurring phosphodiester backbone of an oligonucleotide or polynucleotide can be partially or completely modified with phosphorothioate, phosphorodithioate, or methylphosphonate internucleotide linkage modifications, modified nucleoside bases or modified sugars can be used in oligonucleotide or polynucleotide synthesis, and oligonucleotides or polynucleotides can be labeled with a fluorescent moiety (for example, fluorescein or rhodamine) or other label (for example, biotin).

The polynucleotides can be single- or double-stranded RNA or single- or double-stranded DNA or double-stranded DNA/RNA hybrids or modified analogues thereof, and can be of oligonucleotide lengths or longer. In more specific embodiments of the invention the polynucleotides that provide single-stranded RNA in the plant cell are selected from the group consisting of (a) a single-stranded RNA molecule (ssRNA), (b) a single-stranded RNA molecule that self-hybridizes to form a double-stranded RNA molecule, (c) a double-stranded RNA molecule (dsRNA), (d) a single-stranded DNA molecule (ssDNA), (e) a single-stranded DNA molecule that self-hybridizes to form a double-stranded DNA molecule, and (f) a single-stranded DNA molecule including a modified Pol III gene that is transcribed to an RNA molecule, (g) a double-stranded DNA molecule (dsDNA), (h) a double-stranded DNA molecule including a modified Pol III gene that is transcribed to an RNA molecule, (i) a double-stranded, hybridized RNA/DNA molecule, or combinations thereof. In some embodiments these polynucleotides include chemically modified nucleotides or non-canonical nucleotides. In embodiments of the method the polynucleotides include double-stranded DNA formed by intramolecular hybridization, double-stranded DNA formed by intermolecular hybridization, double-stranded RNA formed by intramolecular hybridization, or double-stranded RNA formed by intermolecular hybridization. In one embodiment the polynucleotides include single-stranded DNA or single-stranded RNA that self-hybridizes to form a hairpin structure having an at least partially double-stranded structure including at least one segment that will hybridize to RNA transcribed from the gene targeted for suppression. Not intending to be bound by any mechanism, it is believed that such polynucleotides are or will produce single-stranded RNA with at least one segment that will hybridize to RNA transcribed from the gene targeted for suppression. In certain other embodiments the polynucleotides further includes a promoter, generally a promoter functional in a plant, for example, a pol II promoter, a pol III promoter, a pol IV promoter, or a pol V promoter.

The term “gene” refers to components that comprise chromosomal DNA, plasmid DNA, cDNA, intron and exon DNA, artificial DNA polynucleotide, or other DNA that encodes a peptide, polypeptide, protein, or RNA transcript molecule, and the genetic elements flanking the coding sequence that are involved in the regulation of expression, such as, promoter regions, 5′ leader regions, 3′ untranslated region that may exist as native genes or transgenes in a plant genome. The gene or a fragment thereof is isolated and subjected to polynucleotide sequencing methods that determines the order of the nucleotides that comprise the gene. Any of the components of the gene are potential targets for a trigger oligonucleotide and polynucleotides.

The trigger polynucleotide molecules are designed to modulate expression by inducing regulation or suppression of an endogenous ALS gene in a plant and are designed to have a nucleotide sequence essentially identical or essentially complementary to the nucleotide sequence of an endogenous ALS gene of a plant or to the sequence of RNA transcribed from an endogenous ALS gene of a plant, including a transgene in a plant that provides for a herbicide resistant ALS enzyme, which can be coding sequence or non-coding sequence. Effective molecules that modulate expression are referred to as “a trigger molecule, or trigger polynucleotide”. By “essentially identical” or “essentially complementary” is meant that the trigger polynucleotides (or at least one strand of a double-stranded polynucleotide or portion thereof, or a portion of a single strand polynucleotide) are designed to hybridize to the endogenous gene noncoding sequence or to RNA transcribed (known as messenger RNA or an RNA transcript) from the endogenous gene to effect regulation or suppression of expression of the endogenous gene. Trigger molecules are identified by “tiling” the gene targets with partially overlapping probes or non-overlapping probes of antisense or sense polynucleotides that are essentially identical or essentially complementary to the nucleotide sequence of an endogenous gene. Multiple target sequences can be aligned and sequence regions with homology in common, according to the methods of the present invention, are identified as potential trigger molecules for the multiple targets. Multiple trigger molecules of various lengths, for example 18-25 nucleotides, 26-50 nucleotides, 51-100 nucleotides, 101-200 nucleotides, 201-300 nucleotides or more can be pooled into a few treatments in order to investigate polynucleotide molecules that cover a portion of a gene sequence (for example, a portion of a coding versus a portion of a noncoding region, or a 5′ versus a 3′ portion of a gene) or an entire gene sequence including coding and noncoding regions of a target gene. Polynucleotide molecules of the pooled trigger molecules can be divided into smaller pools or single molecules in order to identify trigger molecules that provide the desired effect.

The target gene RNA and DNA polynucleotide molecules (Table 1, SEQ ID NO:1-45 and 1692-1788) are sequenced by any number of available methods and equipment. Some of the sequencing technologies are available commercially, such as the sequencing-by-hybridization platform from Affymetrix Inc. (Sunnyvale, Calif.) and the sequencing-by-synthesis platforms from 454 Life Sciences (Bradford, Conn.), Illumina/Solexa (Hayward, Calif.) and Helicos Biosciences (Cambridge, Mass.), and the sequencing-by-ligation platform from Applied Biosystems (Foster City, Calif.), as described below. In addition to the single molecule sequencing performed using sequencing-by-synthesis of Helicos Biosciences, other single molecule sequencing technologies are encompassed and include the SMRT™. technology of Pacific Biosciences, the Ion Torrent™. technology, and nanopore sequencing being developed for example, by Oxford Nanopore Technologies. An ALStarget gene comprising DNA or RNA can be isolated using primers or probes essentially complementary or essentially homologous to SEQ ID NO:1-45 and 1692-1788 or a fragment thereof. A polymerase chain reaction (PCR) gene fragment can be produced using primers essentially complementary or essentially homologous to SEQ ID NO:1-45 and 1692-1788 or a fragment thereof that is useful to isolate an ALS gene from a plant genome. SEQ ID NO: 1-45 and 1692-1788 or fragments thereof can be used in various sequence capture technologies to isolate additional target gene sequences, for example, including but not limited to Roche NimbleGen® (Madison, Wis.) and Streptavdin-coupled Dynabeads® (Life Technologies, Grand Island, N.Y.) and US20110015084, herein incorporated by reference in its entirety.

Embodiments of functional single-stranded polynucleotides have sequence complementarity that need not be 100 percent, but is at least sufficient to permit hybridization to RNA transcribed from the target gene or DNA of the target gene to form a duplex to permit a gene silencing mechanism. Thus, in embodiments, a polynucleotide fragment is designed to be essentially identical to, or essentially complementary to, a sequence of 18 or more contiguous nucleotides in either the target ALS gene sequence or messenger RNA transcribed from the target gene. By “essentially identical” is meant having 100 percent sequence identity or at least about 83, 84, 85, 86, 87, 88, 89, 90, 91, 92, 93, 94, 95, 96, 97, 98, or 99 percent sequence identity when compared to the sequence of 18 or more contiguous nucleotides in either the target gene or RNA transcribed from the target gene; by “essentially complementary” is meant having 100 percent sequence complementarity or at least about 83, 84, 85, 86, 87, 88, 89, 90, 91, 92, 93, 94, 95, 96, 97, 98, or 99 percent sequence complementarity when compared to the sequence of 18 or more contiguous nucleotides in either the target gene or RNA transcribed from the target gene. In some embodiments, polynucleotide molecules are designed to have 100 percent sequence identity with or complementarity to one allele or one family member of a given target gene (coding or non-coding sequence of a gene for of the present invention); in other embodiments the polynucleotide molecules are designed to have 100 percent sequence identity with or complementarity to multiple alleles or family members of a given target gene.

“Identity” refers to the degree of similarity between two polynucleic acid or protein sequences. An alignment of the two sequences is performed by a suitable computer program. A widely used and accepted computer program for performing sequence alignments is CLUSTALW v1.6 (Thompson, et al. Nucl. Acids Res., 22: 4673-4680, 1994). The number of matching bases or amino acids is divided by the total number of bases or amino acids, and multiplied by 100 to obtain a percent identity. For example, if two 580 base pair sequences had 145 matched bases, they would be 25 percent identical. If the two compared sequences are of different lengths, the number of matches is divided by the shorter of the two lengths. For example, if there are 100 matched amino acids between a 200 and a 400 amino acid protein, they are 50 percent identical with respect to the shorter sequence. If the shorter sequence is less than 150 bases or 50 amino acids in length, the number of matches are divided by 150 (for nucleic acid bases) or 50 (for amino acids), and multiplied by 100 to obtain a percent identity.

Trigger molecules for specific gene family members can be identified from coding and/or non-coding sequences of gene families of a plant or multiple plants, by aligning and selecting 200-300 polynucleotide fragments from the least homologous regions amongst the aligned sequences and evaluated using topically applied polynucleotides (as sense or anti-sense ssDNA or ssRNA, dsRNA, or dsDNA) to determine their relative effectiveness in inducing the herbicidal phenotype. The effective segments are further subdivided into 50-60 polynucleotide fragments, prioritized by least homology, and reevaluated using topically applied polynucleotides. The effective 50-60 polynucleotide fragments are subdivided into 19-30 polynucleotide fragments, prioritized by least homology, and again evaluated for induction of the herbicidal phenotype. Once relative effectiveness is determined, the fragments are utilized singly, or again evaluated in combination with one or more other fragments to determine the trigger composition or mixture of trigger polynucleotides for providing the herbicidal phenotype.

Trigger molecules for broad activity can be identified from coding and/or non-coding sequences of gene families of a plant or multiple plants, by aligning and selecting 200-300 polynucleotide fragments from the most homologous regions amongst the aligned sequences and evaluated using topically applied polynucleotides (as sense or anti-sense ssDNA or ssRNA, dsRNA, or dsDNA) to determine their relative effectiveness in inducing the herbicidal phenotype. The effective segments are subdivided into 50-60 polynucleotide fragments, prioritized by most homology, and reevaluated using topically applied polynucleotides. The effective 50-60 polynucleotide fragments are subdivided into 19-30 polynucleotide fragments, prioritized by most homology, and again evaluated for induction of the herbicidal phenotype. Once relative effectiveness is determined, the fragments may be utilized singly, or in combination with one or more other fragments to determine the trigger composition or mixture of trigger polynucleotides for providing the herbicidal phenotype.

Methods of making polynucleotides are well known in the art. Chemical synthesis, in vivo synthesis and in vitro synthesis methods and compositions are known in the art and include various viral elements, microbial cells, modified polymerases, and modified nucleotides. Commercial preparation of oligonucleotides often provides two deoxyribonucleotides on the 3′ end of the sense strand. Long polynucleotide molecules can be synthesized from commercially available kits, for example, kits from Applied Biosystems/Ambion (Austin, Tex.) have DNA ligated on the 5′ end in a microbial expression cassette that includes a bacterial T7 polymerase promoter that makes RNA strands that can be assembled into a dsRNA and kits provided by various manufacturers that include T7 RiboMax Express (Promega, Madison, Wis.), AmpliScribe T7-Flash (Epicentre, Madison, Wis.), and TranscriptAid T7 High Yield (Fermentas, Glen Burnie, Md.). dsRNA molecules can be produced from microbial expression cassettes in bacterial cells (Ongvarrasopone et al. ScienceAsia 33:35-39; Yin, Appl. Microbiol. Biotechnol 84:323-333, 2009; Liu et al., BMC Biotechnology 10:85, 2010) that have regulated or deficient RNase III enzyme activity or the use of various viral vectors to produce sufficient quantities of dsRNA. ALS gene fragments are inserted into the microbial expression cassettes in a position in which the fragments are express to produce ssRNA or dsRNA useful in the methods described herein to regulate expression on a target ALS gene. Long polynucleotide molecules can also be assembled from multiple RNA or DNA fragments. In some embodiments design parameters such as Reynolds score (Reynolds et al. Nature Biotechnology 22, 326-330 (2004), Tuschl rules (Pei and Tuschl, Nature Methods 3(9): 670-676, 2006), i-score (Nucleic Acids Res 35: e123, 2007), i-Score Designer tool and associated algorithms (Nucleic Acids Res 32: 936-948, 2004. Biochem Biophys Res Commun 316: 1050-1058, 2004, Nucleic Acids Res 32: 893-901, 2004, Cell Cycle 3: 790-5, 2004, Nat Biotechnol 23: 995-1001, 2005, Nucleic Acids Res 35: e27, 2007, BMC Bioinformatics 7: 520, 2006, Nucleic Acids Res 35: e123, 2007, Nat Biotechnol 22: 326-330, 2004) are known in the art and may be used in selecting polynucleotide sequences effective in gene silencing. In some embodiments the sequence of a polynucleotide is screened against the genomic DNA of the intended plant to minimize unintentional silencing of other genes.

Ligands can be tethered to a polynucleotide, for example a dsRNA, ssRNA, dsDNA or ssDNA. Ligands in general can include modifiers, e.g., for enhancing uptake; diagnostic compounds or reporter groups e.g., for monitoring distribution; cross-linking agents; nuclease-resistance conferring moieties; and natural or unusual nucleobases. General examples include lipophiles, lipids (e.g., cholesterol, a bile acid, or a fatty acid (e.g., lithocholic-oleyl, lauroyl, docosnyl, stearoyl, palmitoyl, myristoyl oleoyl, linoleoyl), steroids (e.g., uvaol, hecigenin, diosgenin), terpenes (e.g., triterpenes, e.g., sarsasapogenin, Friedelin, epifriedelanol derivatized lithocholic acid), vitamins (e.g., folic acid, vitamin A, biotin, pyridoxal), carbohydrates, proteins, protein binding agents, integrin targeting molecules, polycationics, peptides, polyamines, and peptide mimics. The ligand may also be a recombinant or synthetic molecule, such as a synthetic polymer, e.g., polyethylene glycol (PEG), PEG-40K, PEG-20K and PEG-5K. Other examples of ligands include lipophilic molecules, e.g, cholesterol, cholic acid, adamantane acetic acid, 1-pyrene butyric acid, dihydrotestosterone, glycerol (e.g., esters and ethers thereof, e.g., C.sub.10, C.sub.11, C.sub.12, C.sub.13, C.sub.14, C.sub.15, C.sub.16, C.sub.17, C.sub.18, C.sub.19, or C.sub.20 alkyl; e.g., lauroyl, docosnyl, stearoyl, oleoyl, linoleoyl 1,3-bis-O(hexadecyl)glycerol, 1,3-bis-O(octaadecyl)glycerol), geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dodecanoyl, lithocholyl, 5.beta.-cholanyl, N,N-distearyl-lithocholamide, 1,2-di-O-stearoylglyceride, dimethoxytrityl, or phenoxazine) and PEG (e.g., PEG-5K, PEG-20K, PEG-40K). Preferred lipophilic moieties include lipid, cholesterols, oleyl, retinyl, or cholesteryl residues.

Conjugating a ligand to a dsRNA can enhance its cellular absorption, lipophilic compounds that have been conjugated to oligonucleotides include 1-pyrene butyric acid, 1,3-bis-O-(hexadecyl)glycerol, and menthol. One example of a ligand for receptor-mediated endocytosis is folic acid. Folic acid enters the cell by folate-receptor-radiated endocytosis. dsRNA compounds bearing folic acid would be efficiently transported into the cell via the folate-receptor-mediated endocytosis. Other ligands that have been conjugated to oligonucleotides include polyethylene glycols, carbohydrate clusters, cross-linking agents, porphyrin conjugates, delivery peptides and lipids such as cholesterol. In certain instances, conjugation of a cationic ligand to oligonucleotides results in improved resistance to nucleases. Representative examples of cationic ligands are propylammonium and dimethylpropylammonium. Interestingly, antisense oligonucleotides were reported to retain their high binding affinity to mRNA when the cationic ligand was dispersed, throughout the oligonucleotide. See M. Manoharan Antisense & Nucleic Acid Drug Development 2002, 12, 103 and references therein.

A biologic delivery can be accomplished by a variety of methods including, without limitation, (1) loading liposomes with a dsRNA acid molecule provided herein and (2) complexing a dsRNA molecule with lipids or liposomes to form nucleic acid-lipid or nucleic acid-liposome complexes. The liposome can be composed of cationic and neutral lipids commonly used to transfect cells in vitro. Cationic lipids can complex (e.g., charge-associate) with negatively charged, nucleic acids to form liposomes. Examples of cationic liposomes include, without limitation, lipofectin, lipofectamine, lipofectace, and DOTAP. Procedures for forming liposomes are well known in the art. Liposome compositions can be formed, for example, from phosphatidylcholine, dimyristoyl phosphatidylcholine, dipalmitoyl phosphatidylcholine, dimyristoyl phosphatidyl glycerol, dioleoyl phosphatidylethanolamine or liposomes comprising dihydrosphingomyelin (DHSM) Numerous lipophilic agents are commercially available, including Lipofectin® (Invitrogen/Life Technologies, Carlsbad, Calif.) and Effectene™ (Qiagen, Valencia, Calif.), In addition, systemic delivery methods can be optimized using commercially available cationic lipids such as DDAB or DOTAP, each of which can be mixed with a neutral lipid such as DOPE or cholesterol. In some eases, liposomes such as those described by Templeton et al. (Nature Biotechnology, 15:647-652 (1997)) can be used. In other embodiments, polycations such as polyethyleneimine can be used to achieve delivery in vivo and ex vivo (Boletta et al., J. Am. Soc. Nephrol. 7:1728 (1996)). Additional information regarding the use of liposomes to deliver nucleic acids can be found in U.S. Pat. No. 6,271,359, PCT Publication WO 96/40964 and Morrissey, D. et al. 2005. Nat. Biotechnol. 23(8):1002-7.

In certain embodiments, an organosilicone preparation that is commercially available as Silwet® L-77 surfactant having CAS Number 27306-78-1 and EPA Number: CAL.REG.NO. 5905-50073-AA, and currently available from Momentive Performance Materials, Albany, N.Y. can be used to prepare a polynucleotide composition. In certain embodiments where a Silwet L-77 organosilicone preparation is used as a pre-spray treatment of plant leaves or other plant surfaces, freshly made concentrations in the range of about 0.015 to about 2 percent by weight (wt percent) (e.g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent) are efficacious in preparing a leaf or other plant surface for transfer of polynucleotide molecules into plant cells from a topical application on the surface. In certain embodiments of the methods and compositions provided herein, a composition that comprises a polynucleotide molecule and an organosilicone preparation comprising Silwet L-77 in the range of about 0.015 to about 2 percent by weight (wt percent) (e.g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent) is used or provided.

In certain embodiments, any of the commercially available organosilicone preparations provided such as the following Breakthru S 321, Breakthru S 200 Cat#67674-67-3, Breakthru OE 441 Cat#68937-55-3, Breakthru S 278 Cat #27306-78-1, Breakthru S 243, Breakthru S 233 Cat#134180-76-0, available from manufacturer Evonik Goldschmidt (Germany), Silwet® HS 429, Silwet® HS 312, Silwet® HS 508, Silwet® HS 604 (Momentive Performance Materials, Albany, N.Y.) can be used as transfer agents in a polynucleotide composition. In certain embodiments where an organosilicone preparation is used as a pre-spray treatment of plant leaves or other surfaces, freshly made concentrations in the range of about 0.015 to about 2 percent by weight (wt percent) (e.g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent) are efficacious in preparing a leaf or other plant surface for transfer of polynucleotide molecules into plant cells from a topical application on the surface. In certain embodiments of the methods and compositions provided herein, a composition that comprises a polynucleotide molecule and an organosilicone preparation in the range of about 0.015 to about 2 percent by weight (wt percent) (e.g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent) is used or provided.

Organosilicone preparations used in the methods and compositions provided herein can comprise one or more effective organosilicone compounds. As used herein, the phrase “effective organosilicone compound” is used to describe any organosilicone compound that is found in an organosilicone preparation that enables a polynucleotide to enter a plant cell. In certain embodiments, an effective organosilicone compound can enable a polynucleotide to enter a plant cell in a manner permitting a polynucleotide mediated suppression of a target gene expression in the plant cell. In general, effective organosilicone compounds include, but are not limited to, compounds that can comprise: i) a trisiloxane head group that is covalently linked to, ii) an alkyl linker including, but not limited to, an n-propyl linker, that is covalently linked to, iii) a poly glycol chain, that is covalently linked to, iv) a terminal group. Trisiloxane head groups of such effective organosilicone compounds include, but are not limited to, heptamethyltrisiloxane. Alkyl linkers can include, but are not limited to, an n-propyl linker Poly glycol chains include, but are not limited to, polyethylene glycol or polypropylene glycol. Poly glycol chains can comprise a mixture that provides an average chain length “n” of about “7.5”. In certain embodiments, the average chain length “n” can vary from about 5 to about 14. Terminal groups can include, but are not limited to, alkyl groups such as a methyl group. Effective organosilicone compounds are believed to include, but are not limited to, trisiloxane ethoxylate surfactants or polyalkylene oxide modified heptamethyl trisiloxane.

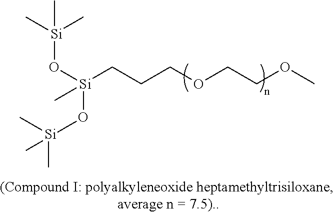

In certain embodiments, an organosilicone preparation that comprises an organosilicone compound comprising a trisiloxane head group is used in the methods and compositions provided herein. In certain embodiments, an organosilicone preparation that comprises an organosilicone compound comprising a heptamethyltrisiloxane head group is used in the methods and compositions provided herein. In certain embodiments, an organosilicone composition that comprises Compound I is used in the methods and compositions provided herein. In certain embodiments, an organosilicone composition that comprises Compound I is used in the methods and compositions provided herein. In certain embodiments of the methods and compositions provided herein, a composition that comprises a polynucleotide molecule and one or more effective organosilicone compound in the range of about 0.015 to about 2 percent by weight (wt percent) (e.g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent) is used or provided.

Compositions of the present invention include but are not limited components that are one or more polynucleotides essentially identical to, or essentially complementary to an ALS gene sequence (promoter, intron, exon, 5′ untranslated region, 3′ untranslated region), a transfer agent that provides for the polynucleotide to enter a plant cell, a herbicide that complements the action of the polynucleotide, one or more additional herbicides that further enhance the herbicide activity of the composition or provide an additional mode of action different from the complementing herbicide, various salts and stabilizing agents that enhance the utility of the composition as an admixture of the components of the composition.

In certain aspects, methods include one or more applications of a polynucleotide composition and one or more applications of a transfer agent for conditioning of a plant to permeation by polynucleotides. When the agent for conditioning to permeation is an organosilicone composition or compound contained therein, embodiments of the polynucleotide molecules are double-stranded RNA oligonucleotides, single-stranded RNA oligonucleotides, double-stranded RNA polynucleotides, single-stranded RNA polynucleotides, double-stranded DNA oligonucleotides, single-stranded DNA oligonucleotides, double-stranded DNA polynucleotides, single-stranded DNA polynucleotides, chemically modified RNA or DNA oligonucleotides or polynucleotides or mixtures thereof.

Compositions and methods of the invention are useful for modulating the expression of an endogenous ALS gene or transgenic ALS gene (for example, U.S. Pat. No. 7,973,218; SEQ ID NO:65 comprising a soybean HRA sequence; SEQ ID NO:66 comprising a maize HRA sequence; SEQ ID NO:67 comprising an Arabidopsis HRA sequence; and SEQ ID NO:86 comprising an HRA sequence used in cotton, herein incorporated by reference) gene in a plant cell. In various embodiments, an ALS gene includes coding (protein-coding or translatable) sequence, non-coding (non-translatable) sequence, or both coding and non-coding sequence. Compositions can include polynucleotides and oligonucleotides designed to target multiple genes, or multiple segments of one or more genes. The target gene can include multiple consecutive segments of a target gene, multiple non-consecutive segments of a target gene, multiple alleles of a target gene, or multiple target genes from one or more species.

Provided is a method for modulating expression of an ALS gene in a plant including (a) conditioning of a plant to permeation by polynucleotides and (b) treatment of the plant with the polynucleotide molecules, wherein the polynucleotide molecules include at least one segment of 18 or more contiguous nucleotides cloned from or otherwise identified from the target ALS gene in either anti-sense or sense orientation, whereby the polynucleotide molecules permeate the interior of the plant and induce modulation of the target gene. The conditioning and polynucleotide application can be performed separately or in a single step. When the conditioning and polynucleotide application are performed in separate steps, the conditioning can precede or can follow the polynucleotide application within minutes, hours, or days. In some embodiments more than one conditioning step or more than one polynucleotide molecule application can be performed on the same plant. In embodiments of the method, the segment can be cloned or identified from (a) coding (protein-encoding), (b) non-coding (promoter and other gene related molecules), or (c) both coding and non-coding parts of the target gene. Non-coding parts include DNA, such as promoter regions or the RNA transcribed by the DNA that provide RNA regulatory molecules, including but not limited to: introns, 5′ or 3′ untranslated regions, and microRNAs (miRNA), trans-acting siRNAs, natural anti-sense siRNAs, and other small RNAs with regulatory function or RNAs having structural or enzymatic function including but not limited to: ribozymes, ribosomal RNAs, t-RNAs, aptamers, and riboswitches.

Herbicide chemical families that are known as ALS inhibiting herbicides include members of the Sulfonylureas, Imidazolinones, Triazolopyrimidines, Pyrimidinyl(thio)benzoates, and Sulfonylaminocarbonyl-triazolinones.

All publications, patents and patent applications are herein incorporated by reference to the same extent as if each individual publication or patent application was specifically and individually indicated to be incorporated by reference.

The following examples are included to demonstrate examples of certain preferred embodiments of the invention. It should be appreciated by those of skill in the art that the techniques disclosed in the examples that follow represent approaches the inventors have found function well in the practice of the invention, and thus can be considered to constitute examples of preferred modes for its practice. However, those of skill in the art should, in light of the present disclosure, appreciate that many changes can be made in the specific embodiments that are disclosed and still obtain a like or similar result without departing from the spirit and scope of the invention.

### EXAMPLES

### Example 1

### Polynucleotides Related to the ALS Gene Sequences

The target ALS polynucleotide molecule is represented by a large subunit and a small subunit gene that naturally occurs in the genome of Amaranthus palmeri, Amaranthus rudis, Amaranthus chlorostachys, Amaranthus graecizans, Amaranthus hybridus, Amaranthus lividus, Amaranthus spinosus, Amaranthus thunbergii, Amaranthus viridis, Ambrosia trifida, Kochia scoparia, Abutilon theophrasti, Chenopodium album, Commelina diffusa, Conyza candensis Digitaria sanguinalis, Euphorbia heterophylla, Lolium multiflorum and include polynucleotide molecules related to the expression of a polypeptide identified as an ALS large subunit and ALS small subunit, that include regulatory molecules, cDNAs comprising coding and noncoding regions of an ALS large subunit gene and fragments thereof and ALS small subunit gene and fragments thereof as shown in Table 1 and SEQ ID NO:1-45 and 1692-1788.

Polynucleotide molecules were extracted from these plant species by methods standard in the field, for example, total RNA was extracted using Trizol Reagent (Invitrogen Corp, Carlsbad, Calif. Cat. No. 15596-018), following the manufacturer's protocol or modifications thereof by those skilled in the art of polynucleotide extraction that may enhance recover or purity of the extracted RNA. Briefly, start with 1 gram of ground plant tissue for extraction. Prealiquot 10 milliliters (mL) Trizol reagent to 15 mL conical tubes. Add ground powder to tubes and shake to homogenize. Incubate the homogenized samples for 5 minutes (min) at room temperature (RT) and then add 3 mL of chloroform. Shakes tubes vigorously by hand for 15-30 seconds (sec) and incubate at RT for 3 min. Centrifuge the tubes at 7,000 revolutions per minute (rpm) for 10 min at 4 degrees C. Transfer the aqueous phase to a new 1.5 mL tube and add 1 volume of cold isopropanol. Incubate the samples for 20-30 min at RT and centrifuge at 10,000 rpm for 10 min at 4 degrees C. Wash pellet with Sigma-grade 80 percent ethanol. Remove the supernatant and briefly air-dry the pellet. Dissolve the RNA pellet in approximately 200 microliters of DEPC treated water. Heat briefly at 65 degrees C. to dissolve pellet and vortex or pipet to resuspend RNA pellet. Adjust RNA concentration to 1-2 microgram/microliter.

DNA was extracted using EZNA SP Plant DNA Mini kit (Omega Biotek, Norcross Ga., Cat#D5511) and Lysing Matrix E tubes (Q-Biogen, Cat#6914), following the manufacturer's protocol or modifications thereof by those skilled in the art of polynucleotide extraction that may enhance recover or purity of the extracted DNA. Briefly, aliquot ground tissue to a Lysing Matrix E tube on dry ice, add 800 μl Buffer SP1 to each sample, homogenize in a bead beater for 35-45 sec, incubate on ice for 45-60 sec, centrifuge at ≧14000 rpm for 1 min at RT, add 10 microliter RNase A to the lysate, incubate at 65° C. for 10 min, centrifuge for 1 min at RT, add 280 μl Buffer SP2 and vortex to mix, incubate the samples on ice for 5 min, centrifuge at ≧10,000 g for 10 min at RT, transfer the supernatant to a homogenizer column in a 2 ml collection tube, centrifuge at 10,000 g for 2 min at RT, transfer the cleared lysate into a 1.5 ml microfuge tube, add 1.5 volumes Buffer SP3 to the cleared lysate, vortex immediately to obtain a homogeneous mixture, transfer up to 650 μl supernatant to the Hi-Bind column, centrifuge at 10,000 g for 1 min, repeat, apply 100 μl 65° C. Elution Buffer to the column, centrifuge at 10,000 g for 5 min at RT.

Next-generation DNA sequencers, such as the 454-FLX (Roche, Branford, Conn.), the SOLiD (Applied Biosystems,), and the Genome Analyzer (HiSeq2000, Illumina, San Diego, Calif.) were used to provide polynucleotide sequence from the DNA and RNA extracted from the plant tissues. Raw sequence data was assembled into contigs. The contig sequence was used to identify trigger molecules that can be applied to the plant to enable regulation of the gene expression.

### Example 2

### Polynucleotides Related to the ALS Gene Trigger Molecules

The gene sequences and fragments of Table 1 were divided into 200 polynucleotide (200-mer) lengths with 25 polynucleotide overlapping regions (SEQ ID NO: 46-1363 and 1789-4166). These polynucleotides are tested to select the most efficacious trigger regions across the length of any target sequence. The trigger polynucleotides are constructed as sense or anti-sense ssDNA or ssRNA, dsRNA, or dsDNA, or dsDNA/RNA hybrids and combined with an organosilicone based transfer agent to provide a polynucleotide preparation. The polynucleotides are combined into sets of two to three polynucleotides per set, using 4-8 nmol of each polynucleotide. Each polynucleotide set is prepared with the transfer agent and applied to a plant or a field of plants in combination with a ALS inhibitor herbicide, or followed by a ALS inhibitor treatment one to three days after the polynucleotide application, to determine the effect on the plant's susceptibility to ALS inhibitor. The effect is measured as stunting the growth and/or killing of the plant and is measured 8-14 days after treatment with the polynucleotide set and ALS inhibitor. The most efficacious sets are identified and the individual polynucleotides are tested in the same methods as the sets are and the most efficacious single 200-mer identified. The 200-mer sequence is divided into smaller sequences of 50-70-mer regions with 10-15 polynucleotide overlapping regions and the polynucleotides tested individually. The most efficacious 50-70-mer is further divided into smaller sequences of 25-mer regions with a 12 to 13 polynucleotide overlapping region and tested for efficacy in combination with ALS inhibitor treatment. By this method it is possible to identify an oligonucleotide or several oligonucleotides that are the most efficacious trigger molecule to effect plant sensitivity to an ALS inhibitor or modulation of ALS gene expression. The modulation of ALS gene expression is determined by the detection of ALS siRNA molecules specific to ALS gene or by an observation of a reduction in the amount of ALS RNA transcript produced relative to an untreated plant or by merely observing the anticipated phenotype of the application of the trigger with an ALS inhibiting herbicide. Detection of siRNA can be accomplished, for example, using kits such as mirVana (Ambion, Austin Tex.) and mirPremier (Sigma-Aldrich, St Louis, Mo.).

The gene sequences and fragments of Table 1 are compared and 21-mers of contiguous polynucleotides are identified that have homology across the various ALS gene sequences. The purpose is to identify trigger molecules that are useful as herbicidal molecules or in combination with an ALS inhibitor herbicide across a broad range of weed species. The sequences SEQ ID NO: 1364-1691 and 4167-4201 represent the 21-mers that are present in the ALS large subunit and small subunit genes of at least seven of the weed species of Table 1. It is contemplated that additional 21-mers can be selected from the sequences of Table 1 that are specific for a single weed species or a few weeds species within a genus or trigger molecules that are at least 18 contiguous nucleotides, at least 19 contiguous nucleotides, at least 20 contiguous nucleotides or at least 21 contiguous nucleotides in length and at least 85 percent identical to an ALS large subuintgene sequence or ALS small subunit gene sequence or a combination of both selected from the group consisting of SEQ ID NO:1-45 and 1692-1788.

By this method it is possible to identify an oligonucleotide or several oligonucleotides that are the most efficacious trigger molecule to effect plant sensitivity to an ALS inhibitor or modulation of ALS gene expression. The modulation of ALS gene expression is determined by the detection of ALS siRNA molecules specific to ALS gene or by an observation of a reduction in the amount of ALS RNA transcript produced relative to an untreated plant. Detection of siRNA can be accomplished, for example, using kits such as mirVana (Ambion, Austin Tex.) and mirPremier (Sigma-Aldrich, St Louis, Mo.).

### Example 3

### Methods Related to Treating Plants or Plant Parts with a Topical Mixture of the Trigger Molecules for ALS Gene Expression Modulation

Single stranded or double stranded DNA or RNA fragments in sense or antisense orientation are identified and mixed with a transfer agent and other components in the composition of the invention. This composition is topically applied to plants to effect expression of the target ALS genes in the specified plant to obtain the desired effect on growth or development.

In this example, growing Amaranthus palmeri plants were treated with a topically applied composition for inducing modulation of a target gene in a plant including (a) an agent for conditioning of a plant to transfer of the polynucleotides and (b) polynucleotides including at least one ssDNA polynucleotide strand including at least one segment of 18-21 contiguous nucleotides of the target gene in sense (S) orientation. Amaranthus palmeri plants were treated with a topically applied adjuvant solution comprising a pool of ssDNA ALS2 oligonucleotides shown in Table 2 that are essentially homologous or essentially complementary to the Amaranthus palmeri ALS2 gene promoter sequence, 0.5% Silwet L-77 solution, 2% ammonium sulfate and 20 mM sodium phosphate buffer (pH 6.8). The name of the each trigger molecule is in the left column and the sequence of the trigger molecule is shown in the right column.

TABLE 2
ssDNA-ALS2 promoter trigger polynucleotides

| Oligo name | DNA sequence | SEQ ID NO: |
|---|---|---|
| ALS_PRO_S1 | TCTTCTCCGACTCTCACAA | 4202 |
| ALS_PRO_S2 | TCTTCCACCCTCTCTAATG | 4203 |
| ALS_PRO_S3 | GGTGGAAAGATTGGAACTT | 4204 |
| ALS_PRO_S4 | TCGTTTGTGGGTTTGTAAG | 4205 |
| ALS_PRO_S5 | GCAATGGAAGTTTCTGCAA | 4206 |
| ALS_PRO_S6 | AGTTCCTGTTTCAGCTCAT | 4207 |
| ALS_PRO_S7 | TGTATGTCAAGGTTTAGGTTG | 4208 |
| ALS_PRO_S8 | GCAATAAGGTGATGGCGTG | 4209 |
| ALS_PRO_S9 | GCGCCTCCACTATCTTCTT | 4210 |
| ALS_PRO_S10 | GCTTTCCTCTCGCACTAAT | 4211 |
| ALS_PRO_S11 | CCATTTACGCTATCCCTTT | 4212 |
| ALS_PRO_S12 | CCCACTTCTTCTTCTTCAA | 4213 |
| ALS_PRO_S13 | CCTAAACCTAAACCTCCTT | 4214 |
| ALS_PRO_S14 | TGTTCTCGTTGAAGCTCTT | 4215 |
| ALS_PRO_S15 | GGAAATCCATCAAGCTCTT | 4216 |
| ALS_PRO_S16 | GGAGTTTGTATTGCCACTT | 4217 |

Approximately four-week old greenhouse grown Amaranthus palmeri plants (glyphosate-resistant Palmer amaranth, “R-22”) were used in this assays. The plants were spray treated in a greenhouse with the ssDNA ALS2 oligonucleotide composition/0.5% Silwet L-77 solution/2% ammonium sulfate/20 mM sodium phosphate, concentration of the each oligonucleotide was approximately 16 nmol. Spray solutions were prepared the same day as spraying and applied using a track sprayer with a FLATfan nozzle 9501E at 165 psi (pounds/square inch) at a rate of 93 g/ha (grams/hectare). The ssDNA ALS2 oligonucleotide composition was applied and followed 24 hours later by the herbicide treatment, Staple® (2-Chloro-6-{(4,6-dimethoxy-2-pyrimidinyl)thio}benzoic acid sodium salt, pyrithiobac sodium 85 percent, Dupont, Wilmington, Del.) at ¼×(27 g ai/ha (grams active ingredient per hectare) rate for the ssDNA ALS2 oligonucleotide treated plants and 1× rate (108 g ai/ha) for the untreated plants, five replications per treatment and the data is presented as percentage height of the untreated control. Plant height is determined at seven days after herbicide treatment.

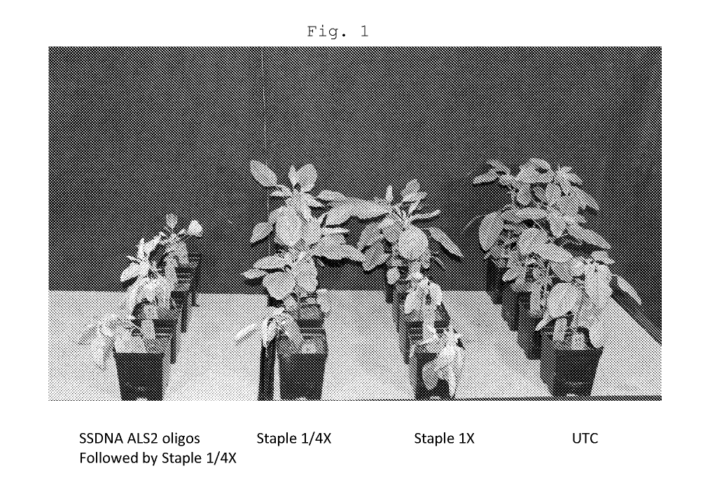

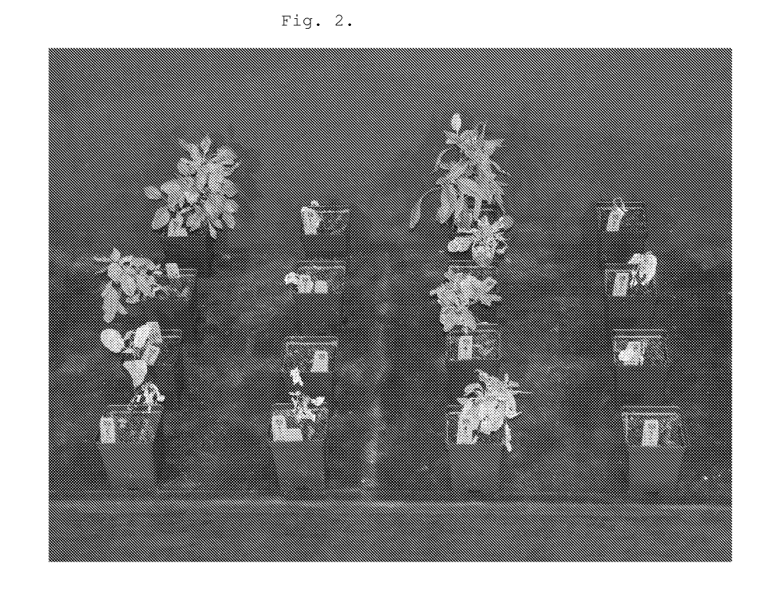

The resulting plants treated with Staple® alone at 27 g ai/ha (¼× field rate) showed moderate growth stunting, causing between 30 percent and 35 percent growth reduction compared to the untreated control. Plants treated with Staple® at 108 g ai/ha (1× field rate) showed 40 percent growth reduction. Plants treated with ssDNA ALS2 oligonucleotides followed by Staple® at 27 g ai/ha at 1 h or 24 h showed a significant increase in growth stunting compared to Staple® alone. Thus, ssDNA ALS2 oligonucleotide treatment followed by Staple® at 1 h and 24 h caused 50 percent and 60 percent growth reduction, relative to the untreated control, while Staple® alone at 27 (1 h and 24 h) and 108 (24 h) g ai/ha caused 35 percent, 30 percent, and 40 percent growth reduction, respectively. A photograph of the treated plants is shown in FIG. 1. Individual trigger molecules enhancing ALS inhibitor herbicide enhancing activity were selected by repeating the testing with the individual oligonucleotides or combinations thereof, for example, ALS_pro 51 (SEQ ID NO:4202) was determined to be an effective trigger molecule. ALS_pro 51 dsDNA trigger molecule was applied to ALS inhibitor resistant Palmer amaranth plants (A3, ALS inhibitor herbicide biotype) followed by ALS inhibitor herbicides 1× Staple® at 108 g ai/ha or 2× Classic® (Chlorimuron Ethyl 25DF, Dupont, Willmington, Del., 2× rate is 70 g ai/ha) at +COC (crop oil concentrate) 24 hr after trigger application. The results of the treatment demonstrates that the ALS_pro_S1 trigger molecule substantially improved the activity of the Staple® and Classic® herbicides on ALS inhibitor resistant plants, FIG. 2.

### Example 4

### Tiling Test of Pooled Trigger Molecule to ALS Large Subunit

Pools of trigger molecules (ALStile) were tested across the ALS large subunit gene to select for effective trigger molecules. Approximately four-week old (3-4 inches tall) greenhouse grown Amaranthus palmeri plants (Palmer amaranth A3) were used in these assays. The plants were spray treated in a greenhouse with the ALStile composition, each composition having six trigger oligonucleotides (Table 3) and a formulation comprising 1 percent Silwet L-77 solution, 2 percent ammonium sulfate and 20 mM sodium phosphate and the concentration of each oligonucleotide was approximately 4 nmol. Spray solutions were prepared the same day as spraying and applied using a track sprayer with a FLATfan nozzle 9501E at 165 psi (pounds/square inch) at a rate of 93 g/ha (grams/hectare). The dsDNA ALStile pool oligonucleotide composition was applied and followed 24 hours later by the herbicide treatment, Staple® (2-Chloro-6-{(4,6-dimethoxy-2-pyrimidinyl)thio}benzoic acid sodium salt, pyrithiobac sodium 85 percent, Dupont, Wilmington, Del.) at 108 g/ha (grams per hectare), five replications per treatment and the data is presented as a visual score of percentage injury determined fourteen days after herbicide application.

The results of these test identified three pools with herbicide enhancing activity relative to the formulation control on the A3 biotype, these were ALStile13-18 (SEQ ID NO:4218-4223), ALStile19-24 (SEQ ID NO:4224-4229) and ALStile61-66 (SEQ ID NO:4230-4235) and ALSpro_S1 (SEQ ID NO:4202, identified as an effective trigger from Example 3). Individual effective trigger molecules can be isolated from the pools and combined as necessary with other selected effective trigger molecules to enhance ALS inhibiting herbicide activity.

TABLE 3
Trigger molecule tiling test for Palmer amaranth ALS large subunit gene

| Trigger | type | Herbicide | Rep1 | Rep2 | Rep3 | Rep4 | Rep5 | avg |
|---|---|---|---|---|---|---|---|---|
| ALStile1-6 | dsDNA | Staple @ 108 g/ha | 20 | 35 | 60 | 20 | 15 | 30 |
| ALStile7-12 | dsDNA | Staple @ 108 g/ha | 25 | 80 | 80 | 70 | 20 | 55 |
| ALStile13-18 | dsDNA | Staple @ 108 g/ha | 25 | 90 | 95 | 95 | 80 | 77 |
| ALStile19-24 | dsDNA | Staple @ 108 g/ha | 15 | 80 | 98 | 95 | 95 | 76 |
| ALStile25-30 | dsDNA | Staple @ 108 g/ha | 0 | 35 | 95 | 95 | 85 | 62 |
| ALStile31-36 | dsDNA | Staple @ 108 g/ha | 10 | 5 | 70 | 95 | 10 | 38 |
| ALStile37-42 | dsDNA | Staple @ 108 g/ha | 3 | 3 | 0 | 85 | 75 | 33 |
| ALStile43-48 | dsDNA | Staple @ 108 g/ha | 5 | 8 | 50 | 10 | 0 | 14 |
| ALStile53-54 | dsDNA | Staple @ 108 g/ha | 3 | 15 | 90 | 85 | 10 | 40 |
| ALStile55,57-60 | dsDNA | Staple @ 108 g/ha | 3 | 15 | 98 | 99 | 15 | 46 |
| ALStile61-66 | dsDNA | Staple @ 108 g/ha | 10 | 50 | 99 | 100 | 90 | 70 |
| ALStile67-72 | dsDNA | Staple @ 108 g/ha | 10 | 10 | 85 | 98 | 85 | 57 |
| ALStile73-78 | dsDNA | Staple @ 108 g/ha | 15 | 0 | 60 | 80 | 20 | 35 |
| ALStile79-84 | dsDNA | Staple @ 108 g/ha | 15 | 10 | 15 | 80 | 30 | 30 |
| ALStile85-88 | dsDNA | Staple @ 108 g/ha | 3 | 0 | 0 | 0 | 0 | 0.6 |
| ALStile89-92 | dsDNA | Staple @ 108 g/ha | 0 | 0 | 0 | 0 | 3 | 0.6 |
| ALSpro_S1 | dsRNA | Staple @ 108 g/ha | 100 | 100 | 100 | 100 | 100 | 100 |
| formulation | | Staple @ 108 g/ha | 85 | 80 | 60 | 50 | 10 | 57 |
| Untreated | | | 0 | 0 | 0 | 0 | 0 | 0 |

### Example 5

### Tiling Test of Pooled Trigger Molecules to ALS Small Subunit Gene

Pools of trigger molecules (ALSprotile) were tested to query across the ALS small subunit gene promoter to select for effective trigger molecules. Approximately, 2-4 inches tall R-22 greenhouse grown Amaranthus palmeri plants (R-22) were used in these assays. The plants were spray treated in a greenhouse with the ALSprotile composition, each composition having six trigger oligonucleotides (Table 4) and a formulation comprising 1 percent Silwet L-77 solution, 2 percent ammonium sulfate and 20 mM sodium phosphate and the concentration of each oligonucleotide was approximately 4 nmol. Spray solutions were prepared the same day as spraying and applied using a track sprayer with a FLATfan nozzle 9501E at 165 psi (pounds/square inch) at a rate of 93 g/ha (grams/hectare). The dsDNA ALSprotile pool oligonucleotide composition was applied and followed 24 hours later by the herbicide treatment, Staple® at 108 g/ha (grams per hectare), five replications per treatment and the data is presented as a visual score of percentage injury determined fourteen days after herbicide application. The results of these tests identified two pools with herbicide enhancing activity relative to the formulation control on the A3 biotype, these were ALSprotilel-6 (SEQ ID NO:4236-4241) and ALSprotile7-12 (SEQ ID NO:4242-4247) which demonstrated 67 percent and 62 percent injury, respectfully. Individual effective trigger molecules can be isolated from the pools and combined as necessary with other selected effective trigger molecules to enhance ALS inhibiting herbicide activity.

TABLE 4
Trigger molecule tiling test for Palmer amaranth ALS small subunit gene promoter

| Trigger | type | Herbicide | Rep1 | Rep2 | Rep3 | Rep4 | Rep5 | avg |
|---|---|---|---|---|---|---|---|---|
| ALSprotile1-6 | dsDNA | Staple @ 108 g/ha | 45 | 100 | 50 | 75 | 65 | 67 |
| ALStile7-12 | dsDNA | Staple @ 108 g/ha | 50 | 75 | 20 | 100 | 65 | 62 |
| ALStile13-18 | dsDNA | Staple @ 108 g/ha | 55 | 30 | 65 | 60 | 80 | 58 |
| ALStile19-24 | dsDNA | Staple @ 108 g/ha | 30 | 40 | 75 | 60 | 70 | 55 |
| formulation | | Staple @ 108 g/ha | 60 | 45 | 45 | 55 | 40 | 49 |

### Example 6

### A Method to Control Weeds in a Field

A method to control weeds in a field comprises the use of trigger polynucleotides that can modulate the expression of an ALS gene in one or more target weed plant species. An analysis of ALS gene sequences from eighteen plant species provided a collection of 21-mer polynucleotides that can be used in compositions to affect the growth or develop or sensitivity to ALS inhibitor herbicide to control multiple weed species in a field. A composition containing 1 or 2 or 3 or 4 or more of the polynucleotides of SEQ ID NO: 1364-1691 and 4167-4201 would enable broad activity of the composition against the multiple weed species that occur in a field environment.

The method includes creating a composition that comprises components that include at least one polynucleotide of SEQ ID NO: 1364-1691 and 4167-4201 or any other effective gene expression modulating polynucleotide essentially identical or essentially complementary to SEQ ID NO:1-45 and 1692-1788 or fragment thereof, a transfer agent that mobilizes the polynucleotide into a plant cell and a ALS inhibiting herbicide and optionally a polynucleotide that modulates the expression of an essential gene and optionally a co-herbicide that has a different mode of action relative to an ALS inhibitor herbicide, or a co-herbicide that has a similar mode of action of any one ALS inhibitor herbicide and is a member of a different chemical family. The polynucleotide of the composition includes a dsRNA, ssDNA or dsDNA or a combination thereof. A composition containing a polynucleotide can have a use rate of about 1 to 30 grams or more per acre depending on the size of the polynucleotide and the number of polynucleotides in the composition. The composition may include one or more additional herbicides as needed to provide effective multi-species weed control. For example, a composition comprising an ALS gene trigger oligonucleotide, the composition further including a co-herbicide but not limited to acetochlor, acifluorfen, acifluorfen-sodium, aclonifen, acrolein, alachlor, alloxydim, allyl alcohol, ametryn, amicarbazone, amidosulfuron, aminopyralid, amitrole, ammonium sulfamate, anilofos, asulam, atraton, atrazine, azimsulfuron, BCPC, beflubutamid, benazolin, benfluralin, benfuresate, bensulfuron, bensulfuron-methyl, bensulide, bentazone, benzfendizone, benzobicyclon, benzofenap, bifenox, bilanafos, bispyribac, bispyribac-sodium, borax, bromacil, bromobutide, bromoxynil, butachlor, butafenacil, butamifos, butralin, butroxydim, butylate, cacodylic acid, calcium chlorate, cafenstrole, carbetamide, carfentrazone, carfentrazone-ethyl, CDEA, CEPC, chlorflurenol, chlorflurenol-methyl, chloridazon, chlorimuron, chlorimuron-ethyl, chloroacetic acid, chlorotoluron, chlorpropham, chlorsulfuron, chlorthal, chlorthal-dimethyl, cinidon-ethyl, cinmethylin, cinosulfuron, cisanilide, clethodim, clodinafop, clodinafop-propargyl, clomazone, clomeprop, clopyralid, cloransulam, cloransulam-methyl, CMA, 4-CPB, CPMF, 4-CPP, CPPC, cresol, cumyluron, cyanamide, cyanazine, cycloate, cyclosulfamuron, cycloxydim, cyhalofop, cyhalofop-butyl, 2,4-D, 3,4-DA, daimuron, dalapon, dazomet, 2,4-DB, 3,4-DB, 2,4-DEB, desmedipham, dicamba, dichlobenil, ortho-dichlorobenzene, para-dichlorobenzene, dichlorprop, dichlorprop-P, diclofop, diclofop-methyl, diclosulam, difenzoquat, difenzoquat metilsulfate, diflufenican, diflufenzopyr, dimefuron, dimepiperate, dimethachlor, dimethametryn, dimethenamid, dimethenamid-P, dimethipin, dimethylarsinic acid, dinitramine, dinoterb, diphenamid, diquat, diquat dibromide, dithiopyr, diuron, DNOC, 3,4-DP, DSMA, EBEP, endothal, EPTC, esprocarb, ethalfluralin, ethametsulfuron, ethametsulfuron-methyl, ethofumesate, ethoxyfen, ethoxysulfuron, etobenzanid, fenoxaprop-P, fenoxaprop-P-ethyl, fentrazamide, ferrous sulfate, flamprop-M, flazasulfuron, florasulam, fluazifop, fluazifop-butyl, fluazifop-P, fluazifop-P-butyl, flucarbazone, flucarbazone-sodium, flucetosulfuron, fluchloralin, flufenacet, flufenpyr, flufenpyr-ethyl, flumetsulam, flumiclorac, flumiclorac-pentyl, flumioxazin, fluometuron, fluoroglycofen, fluoroglycofen-ethyl, flupropanate, flupyrsulfuron, flupyrsulfuron-methyl-sodium, flurenol, fluridone, fluorochloridone, fluoroxypyr, flurtamone, fluthiacet, fluthiacet-methyl, fomesafen, foramsulfuron, fosamine, glufosinate, glufosinate-ammonium, halosulfuron, halosulfuron-methyl, haloxyfop, haloxyfop-P, HC-252, hexazinone, imazamethabenz, imazamethabenz-methyl, imazamox, imazapic, imazapyr, imazaquin, imazethapyr, imazosulfuron, indanofan, iodomethane, iodosulfuron, iodosulfuron-methyl-sodium, ioxynil, isoproturon, isouron, isoxaben, isoxachlortole, isoxaflutole, karbutilate, lactofen, lenacil, linuron, MAA, MAMA, MCPA, MCPA-thioethyl, MCPB, mecoprop, mecoprop-P, mefenacet, mefluidide, mesosulfuron, mesosulfuron-methyl, mesotrione, metam, metamifop, metamitron, metazachlor, methabenzthiazuron, methylarsonic acid, methyldymron, methyl isothiocyanate, metobenzuron, metolachlor, S-metolachlor, metosulam, metoxuron, metribuzin, metsulfuron, metsulfuron-methyl, MK-66, molinate, monolinuron, MSMA, naproanilide, napropamide, naptalam, neburon, nicosulfuron, nonanoic acid, norflurazon, oleic acid (fatty acids), orbencarb, orthosulfamuron, oryzalin, oxadiargyl, oxadiazon, oxasulfuron, oxaziclomefone, oxyfluorfen, paraquat, paraquat dichloride, pebulate, pendimethalin, penoxsulam, pentachlorophenol, pentanochlor, pentoxazone, pethoxamid, petrolium oils, phenmedipham, phenmedipham-ethyl, picloram, picolinafen, pinoxaden, piperophos, potassium arsenite, potassium azide, pretilachlor, primisulfuron, primisulfuron-methyl, prodiamine, profluazol, profoxydim, prometon, prometryn, propachlor, propanil, propaquizafop, propazine, propham, propisochlor, propoxycarbazone, propoxycarbazone-sodium, propyzamide, prosulfocarb, prosulfuron, pyraclonil, pyraflufen, pyraflufen-ethyl, pyrazolynate, pyrazosulfuron, pyrazosulfuron-ethyl, pyrazoxyfen, pyribenzoxim, pyributicarb, pyridafol, pyridate, pyriftalid, pyriminobac, pyriminobac-methyl, pyrimisulfan, pyrithiobac, pyrithiobac-sodium, quinclorac, quinmerac, quinoclamine, quizalofop, quizalofop-P, rimsulfuron, sethoxydim, siduron, simazine, simetryn, SMA, sodium arsenite, sodium azide, sodium chlorate, sulcotrione, sulfentrazone, sulfometuron, sulfometuron-methyl, sulfosate, sulfosulfuron, sulfuric acid, tar oils, 2,3,6-TBA, TCA, TCA-sodium, tebuthiuron, tepraloxydim, terbacil, terbumeton, terbuthylazine, terbutryn, thenylchlor, thiazopyr, thifensulfuron, thifensulfuron-methyl, thiobencarb, tiocarbazil, topramezone, tralkoxydim, tri-allate, triasulfuron, triaziflam, tribenuron, tribenuron-methyl, tricamba, triclopyr, trietazine, trifloxysulfuron, trifloxysulfuron-sodium, trifluralin, triflusulfuron, triflusulfuron-methyl, trihydroxytriazine, tritosulfuron, [3-[2-chloro-4-fluoro-5-(-methyl-6-trifluoromethyl-2,4-dioxo-,2,3,44-etrahydropyrimidin-3-yl)phenoxy]-2-pyridyloxy]acetic acid ethyl ester (CAS RN 353292-3-6), 4-[(4,5-dihydro-3-methoxy-4-methyl-5-oxo)-H-,2,4-triazol-1-ylcarbonyl-sulfamoyl]-5-methylthiophene-3-carboxylic acid (BAY636), BAY747 (CAS RN 33504-84-2), topramezone (CAS RN 2063-68-8), 4-hydroxy-3-[[2-[(2-methoxyethoxy)methyl]-6-(trifluoro-methyl)-3-pyridi-nyl]carbonyl]-bicyclo[3.2.]oct-3-en-2-one (CAS RN 35200-68-5), and 4-hydroxy-3-[[2-(3-methoxypropyl)-6-(difluoromethyl)-3-pyridinyl]carbon-yl]-bicyclo[3.2.]oct-3-en-2-one.

A field of crop plants in need of weed plant control is treated by spray application of the composition. The composition can be provided as a tank mix, a sequential treatment of components (generally the polynucleotide followed by the herbicide), a simultaneous treatment or mixing of one or more of the components of the composition from separate containers. Treatment of the field can occur as often as needed to provide weed control and the components of the composition can be adjusted to target specific weed species or weed families.

### Example 7

### Herbicidal Compositions Comprising Pesticidal Agents

A method of controlling weeds and plant pest and pathogens in a field of ALS inhibitor tolerant crop plants is provided, wherein the method comprises applying a composition comprising an ALS trigger polynucleotide, an ALS inhibitor herbicide composition and an admixture of a pest control agent. For example, the admixture comprises insecticides, fungicides, nematocides, bactericides, acaricides, growth regulators, chemosterilants, semiochemicals, repellents, attractants, pheromones, feeding stimulants or other biologically active compounds or biological agents, such as, microorganisms.

For example, the admixture comprises a fungicide compound for use on an ALS herbicide tolerant crop plant to prevent or control plant disease caused by a plant fungal pathogen, The fungicide compound of the admixture may be a systemic or contact fungicide or mixtures of each. More particularly the fungicide compound includes, but is not limited to members of the chemical groups strobilurins, triazoles, chloronitriles, carboxamides and mixtures thereof. The composition may additional have an admixture comprises an insecticidal compound or agent.

Agricultural chemicals are provided in containers suitable for safe storage, transportation and distribution, stability of the chemical compositions, mixing with solvents and instructions for use. A container of the compositions of the present invention may include mixture of a trigger oligonucleotide+ALS inhibitor herbicide+fungicide compound, or a mixture of a trigger oligonucleotide+ALS inhibitor herbicide compound and an insecticide compound, or a trigger oligonucleotide+a ALS inhibitor herbicide compound and a fungicide compound and an insecticide compound (for example, lambda-cyhalothrin, Warrier®). The container may further provide instructions on the effective use of the mixture. Containers of the present invention can be of any material that is suitable for the storage of the chemical mixture. Containers can be of any material that is suitable for the shipment of the chemical mixture. The material can be of cardboard, plastic, metal, or a composite of these materials. The container can have a volume of 0.5 liter, 1 liter, 2 liter, 3-5 liter, 5-10 liter, 10-20 liter, 20-50 liter or more depending upon the need. A tank mix of a trigger oligonucleotide+ALS inhibitor herbicide compound and a fungicide compound is provided, methods of application to the crop to achieve an effective dose of each compound are known to those skilled in the art and can be refined and further developed depending on the crop, weather conditions, and application equipment used.

Insecticides, fungicides, nematocides, bactericides, acaricides, growth regulators, chemosterilants, semiochemicals, repellents, attractants, pheromones, feeding stimulants or other biologically active compounds can be added to the trigger oligonucleotide to form a multi-component pesticide giving an even broader spectrum of agricultural protection. Examples of such agricultural protectants with which compounds of this invention can be formulated are: insecticides such as abamectin, acephate, azinphos-methyl, bifenthrin, buprofezin, carbofuran, chlorfenapyr, chlorpyrifos, chlorpyrifos-methyl, cyfluthrin, beta-cyfluthrin, cyhalothrin, lambda-cyhalothrin, deltamethrin, diafenthiuron, diazinon, diflubenzuron, dimethoate, esfenvalerate, fenoxycarb, fenpropathrin, fenvalerate, fipronil, flucythrinate, tau-fluvalinate, fonophos, imidacloprid, isofenphos, malathion, metaldehyde, methamidophos, methidathion, methomyl, methoprene, methoxychlor, methyl 7-chloro-2,5-dihydro-2-[[N-(methoxycarbonyl)-N-[4-(trifluoromethoxy)phenyl]amino]carbonyl]indeno[1,2-e][1,3,4]oxadiazine-4-a(3H)-carboxylate (DPX-JW062), monocrotophos, oxamyl, parathion, parathion-methyl, permethrin, phorate, phosalone, phosmet, phosphamidon, pirimicarb, profenofos, rotenone, sulprofos, tebufenozide, tefluthrin, terbufos, tetrachlorvinphos, thiodicarb, tralomethrin, trichlorfon and triflumuron; most preferably a glyphosate compound is formulated with a fungicide compound or combinations of fungicides, such as azoxystrobin, benomyl, blasticidin-S, Bordeaux mixture (tribasic copper sulfate), bromuconazole, captafol, captan, carbendazim, chloroneb, chlorothalonil, copper oxychloride, copper salts, cymoxanil, cyproconazole, cyprodinil (CGA 219417), diclomezine, dicloran, difenoconazole, dimethomorph, diniconazole, diniconazole-M, dodine, edifenphos, epoxiconazole (BAS 480F), famoxadone, fenarimol, fenbuconazole, fenpiclonil, fenpropidin, fenpropimorph, fluazinam, fluquinconazole, flusilazole, flutolanil, flutriafol, folpet, fosetyl-aluminum, furalaxyl, hexaconazole, ipconazole, iprobenfos, iprodione, isoprothiolane, kasugamycin, kresoxim-methyl, mancozeb, maneb, mepronil, metalaxyl, metconazole, S-methyl 7-benzothiazolecarbothioate (CGA 245704), myclobutanil, neo-asozin (ferric methanearsonate), oxadixyl, penconazole, pencycuron, probenazole, prochloraz, propiconazole, pyrifenox, pyroquilon, quinoxyfen, spiroxamine (KWG4168), sulfur, tebuconazole, tetraconazole, thiabendazole, thiophanate-methyl, thiram, triadimefon, triadimenol, tricyclazole, trifloxystrobin, triticonazole, validamycin and vinclozolin; combinations of fungicides are common for example, cyproconazole and azoxystrobin, difenoconazole, and metalaxyl-M, fludioxonil and metalaxyl-M, mancozeb and metalaxyl-M, copper hydroxide and metalaxyl-M, cyprodinil and fludioxonil, cyproconazole and propiconazole; commercially available fungicide formulations for control of Asian soybean rust disease include, but are not limited to Quadris® (Syngenta Corp), Bravo® (Syngenta Corp), Echo 720® (Sipcam Agro Inc), Headline® 2.09EC (BASF Corp), Tilt® 3.6EC (Syngenta Corp), PropiMax™ 3.6EC (Dow AgroSciences), Bumper® 41.8EC (MakhteshimAgan), Folicur® 3.6F (Bayer CropScience), Laredo® 25EC (Dow AgroSciences), Laredo™ 25EW (Dow AgroSciences), Stratego® 2.08F (Bayer Corp), Domark™ 125SL (Sipcam Agro USA), and Pristine®38% WDG (BASF Corp) these can be combined with compositions as described in the present invention to provide enhanced protection from soybean rust disease; nematocides such as aldoxycarb and fenamiphos; bactericides such as streptomycin; acaricides such as amitraz, chinomethionat, chlorobenzilate, cyhexatin, dicofol, dienochlor, etoxazole, fenazaquin, fenbutatin oxide, fenpropathrin, fenpyroximate, hexythiazox, propargite, pyridaben and tebufenpyrad; and biological agents such as Bacillus thuringiensis, Bacillus thuringiensis delta endotoxin, baculovirus, and entomopathogenic bacteria, virus and fungi.

TABLE 1
Acetolactate synthase gene cDNA and gDNA contig sequences

| SEQ | | | | |
|---|---|---|---|---|
| ID NO | SPECIES | TYPE | LENGTH | SEQ |
| 1 | Amaranthus | cDNA | 534 | ATAATTATTTGGTGTTAGATGTTGAGGATATCCTAGAATTGTTAAGG |
| | chlorostachys | Contig | | AAGCTTTCTGTTAGCTAATTCTGGTAGACCTGGACCTGTTTTGATTG |
| | | | | ATATCCTAAAGATATTCAGCAACAATTAGTTGTTCCAATTGGGAACA |
| | | | | GCCCAATTAAATTGGGTGGGTATCTTTCTAGGTTGCCTAAACTCACT |
| | | | | TCAACTGCTAATTAAGAGGGACTTCTTGATCAAATTGTGAGGTTGGT |
| | | | | GGGTGAAGAAACCATTGATGAAGATGGGCGAAGAATTCAAATCAA |
| | | | | TGTTGTGGTTTTGAAGGTTTTGTTGTGAAGGTTTTTTTTAAGTGCCA |
| | | | | TTAATGAAGAGGTTTAAGGAAGAAACCATAATGGGGAGGTTGAGG |
| | | | | AAGAAGGTGATGAAGATGATAACCTGCTGTTTCAGGTTACTAAAAC |
| | | | | CGACTACACCTTTAAAAGCGAAGACCCCCAAAGTTCGAGCCTTTAA |
| | | | | AAGATAAACGCAAGTTTAAACCTCTAAAAGCAAATGAGCTAAAGTT |
| | | | | CGTACCTTTTAAATCAAATTTTCC |
| 2 | Amaranthus | cDNA | 500 | CGGGCACATACATACCTCGGGAATCCTTCCAATTCTTCCGAAATCTT |
| | chlorostachys | Contig | | CCCGGATATGCTCAAATTTGCTGAAGCATGTGATATACCAGCAGCC |
| | | | | CGTGTTACCAAGGTGAGCGATTTAAGGACTGCAATTCAAACAATGT |
| | | | | TGGATACTCCAGGACCGTATCTGCTGGATGTAATCGTACCACATCA |
| | | | | GGAGCATGTGCTGCCTATGATCCCTAGCGGTGCCGCCTTCAAGGAC |
| | | | | ACCATAACAGAGGGTGATGGAAGAAGGGCTTATTAGTTGGTTGGA |
| | | | | GATCTTTATAGAGGAGAAGCTTTTTTGTATGTATGTTAGTAGTTCCA |
| | | | | TAAACTTCTATATTCTCTGGCCGTTCTCTCGTTTAGCTGTTTTTATGTT |
| | | | | AGTTTGTTGTTTTCATGTTGCTTGCTACTTTGAAAAACCCTTTTGTGT |
| | | | | TTTAGACCCATTAGCATGAATAATCTTCCTATATTATTGTATGGTTCG |
| | | | | ATACACGCTAGTTGTTTCTTTGTATTATCGAG |
| 3 | Amaranthus | cDNA | 2267 | TTCCTTCTCAGTGATTCCCTTTCTCCATTTTCGCTTAGCTCTCCTCTCA |
| | graecizans | Contig | | CACTAATTACCTCCATTTCCAACCTTCCAAGCTTTCAACAATGGCGTC |
| | | | | CACTTCTTCAAACCCACCATTTTCCTCTTTTACTAAACCTAACAAAAT |
| | | | | CCCTAATCTTCAATCCTCCATTTACGCTCTCCCTTTTTCCAATTCTCTT |
| | | | | AAACCCACTTCTTCTTCTTCAATCCTCCGCCGCCCTCTTCAAATCTCAT |
| | | | | CATCTTCTTCTCAATCACCTAAACCTAAACCTCCTTCCGCTACTATAA |
| | | | | TTCAATCACCATCTCTCACCGATGATAAACCCTCTTCTTTTTTTTCCCG |
| | | | | ATTTAGCCCTGAAGAACCCAGAAAAGGTTGCGATGTTCTTGTTGAA |
| | | | | GCTCTTGAACGTGAAGGTGTTACCGATGTTTTTGCTTACCCTGGTGG |
| | | | | AGCTTCCATGGAAATCCATCAAGCTCTTACTCGTTCTAATATCATTAG |
| | | | | AAATGTTCTTCCTCGACATGAACAAGGTGGGTTTTCGCTGCTGAAG |
| | | | | GCTACGCTCGTGCTACTGGACGTGTTGGAGTTTGTATTGCTACTTCT |
| | | | | GGTCCAGGTGCTACTAATCTTGTCTCTGGTCTTGCTGATGCCCTTCTT |
| | | | | GACTCAGTCCCGCTTGTCGCCATTACTGGGCAAGTTCCTCGGCGTAT |
| | | | | GATCGGTACTGATGCTTTTCAAGAGACTCCTATTGTTGAGGTAACTC |
| | | | | GATCGATTACTAAGCATAATTATCTGGTGTTAGATGTTGAGGATATC |
| | | | | CCTAGAATTGTTAAGGAAGCTTTCTTTTTAGCTAATTCTGGTAGACC |
| | | | | TGGACCTGTTTTGATTGATATTCCTAAAGATATTCAGCAACAATTGG |
| | | | | TTGTTCCTAATTGGGAACAGCCTATTAAATTAGGTGGGTATCTTTCT |
| | | | | AGGTTGCCTAAACCCACTTATTCTGCTAATGAAGAGGGGCTTCTTGA |
| | | | | TCAAATTGTGAGGTTAGTGGGTGAGGCTAAGAGACCTGTGCTGTAT |
| | | | | ACTGGAGGTGGGTGTTTGAATTCTAGTGAAGAATTAAGGAAATTTG |
| | | | | TCGAGTTGACAGGGATTCCGGTTGCTAGTACTTTAATGGGGTTGGG |
| | | | | GGCTTTCCCTTGTACTGATGATTTGTCCCTTCATATGTTGGGAATGC |
| | | | | ACGGGACTGTGTACGCGAATTACGCGGTTGATAAGGCTGATTTATT |
| | | | | GCTTGCTTTCGGGGTTAGGTTTGATGATCGAGTGACTGGGAAGCTT |
| | | | | GAGGCGTTTGCTAGCCGGGCTAAGATTGTGCACATCGATATCGATT |
| | | | | CTGCTGAAATCGGGAAGAATAAGCAACCTCATGTGTCGATTTGTGG |
| | | | | TGATGTTAAAGTGGCATTACAGGGGTTGAATAAGATTTTGGAATCT |
| | | | | AGAAAAGGAAAGGTGAAATTGGATTTCTCTAATTGGAGGGAGGAA |
| | | | | TTGAATGAGCAGAAAAAGAAGTTTCCTTTGAGTTTTAAGACTTTCGG |
| | | | | GGACGCAATTCCTCCGCAATATGCCATTCAGGTGCTTGACGAGTTG |
| | | | | ACAAAGGGTGATGCGGTTGTAAGTACCGGTGTTGGGCAGCACCAA |
| | | | | ATGTGGGCTGCCCAATTCTATAAGTACCGAAATCCTCGCCAATGGCT |
| | | | | GACCTCGGGTGGTTTGGGGGCTATGGGGTTTGGTCTACCAGCCGCT |
| | | | | ATTGGAGCTGCTGTTGCTCGACCAGATGCGGTGGTTGTAGACATTG |
| | | | | ATGGGGATGGGAGTTTTATCATGAATGTTCAAGAATTGGCTACGAT |
| | | | | TAGGGTGGAGAATCTCCCGGTTAAAATCATGCTCTTGAACAATCAA |
| | | | | CATTTAGGTATGGTTGTTCAATGGGAAGATCGATTTTACAAAGCTAA |
| | | | | CCGGGCACATACATACCTCGGGAATCCTTCTAATTCTTCCGAAATCT |
| | | | | TCCCGGATATGCTCAAATTTGCTGAAGCATGTGATATACCAGCAGCC |
| | | | | CGTGTTACCAAGGTGAGCGATTTAAGGGCTGCAATTCAAACAATGT |
| | | | | TGGATACTCCAGGACCATATCTGCTGGATGTAATCGTACCACATCAG |
| | | | | GAGCATGTGCTGCCTATGATCCCTAGCGGCGCCGCCTTCAAGGACA |
| | | | | CCATCACAGAGGGTGATGGAAGAAGGGCTTATTAGTTGGTTGGAG |
| | | | | ATCTTTATAGAGGAGAAGCTTTTTTGTATGTATGTTAGTAGTTCCAT |
| | | | | AAACTTCTATATTCTCTGGCCGTTCTCTCGTTTAGCTGTTTTTATGTTA |
| | | | | GTTTGTTGTTTTCATGTTGCTTGTTACTTTGAAAAACCCTTTTGTGTTT |
| | | | | AAGACCCATTAGCATAG |
| 4 | Amaranthus | cDNA | 2572 | TTACCTTCATTTCCAACCTTTCAAGCTTTCAACAATGGCGTCCACTTC |
| | hybridus | Contig | | TTCAAACCCACCATTTTCCTCTTTTACTAAACCTAACAAAATCCCTAA |
| | | | | TCTGCAATCATCCATTTACGCTATCCCTTTTTCCAATTCTCTTAAACCC |
| | | | | ACTTCTTCTTCTTCAATCCTCCGCCGCCCTCTTCAAATCTCATCATCTT |
| | | | | CTTCTCAATCACCTAAACCTAAACCTCCTTCCGCTACTATAACTCAAT |
| | | | | CACCTTCGTCTCTCACCGATGATAAACCCTCTTCTTTTGTTTCCCGAT |
| | | | | TTAGCCCTGAAGAACCCAGAAAAGGTTGCGATGTTCTCGTTGAAGC |
| | | | | TCTTGAACGTGAAGGTGTTACCGATGTTTTTGCTTACCCTGGTGGAG |
| | | | | CATCCATGGAAATTCATCAAGCTCTTACTCGTTCTAATATCATTAGAA |
| | | | | ATGTTCTTCCTCGACATGAACAAGGTGGGGTTTTCGCTGCTGAAGG |
| | | | | CTACGCTCGTGCTACTGGACGCGTTGGAGTTTGTATTGCCACTTCTG |
| | | | | GTCCAGGTGCTACTAATCTTGTTTCTGGTCTTGCTGATGCACTTCTTG |
| | | | | ACTCAGTCCCTCTTGTCGCCATTACTGGGCAAGTTCCCCGGCGTATG |
| | | | | ATTGGTACTGATGCTTTTCAAGAGACTCCAATTGTTGAGGTAACTCG |
| | | | | ATCCATTACCAAGCATAATTATTTGGTGTTAGATGTTGAGGATATTC |
| | | | | CTAGAATTGTTAAGGAAGCTTTCTTTTTAGCTAATTCTGGTAGACCT |
| | | | | GGACCTGTTTTGATTGATATTCCTAAAGATATTCAGCAACAATTAGT |
| | | | | TGTTCCTAATTGGGAACAGCCCATTAAATTGGGTGGGTATCTTTCTA |
| | | | | GGTTGCCTAAACCCACTTATTCTGCTAATGAAGAGGGACTTCTTGAT |
| | | | | CAAATTGTAAGGTTAGTGGGTGAGTCTAAGAGACCTGTGCTGTATA |
| | | | | CTGGAGGTGGGTGTTTGAATTCTAGTGAAGAATTGAGGAAATTTGT |
| | | | | CGAATTGACAGGTATTCCGGTGGCTAGTACTTTAATGGGGTTGGGG |
| | | | | GCTTTCCCTTGTACTGATGATTTATCTCTTCATATGTTGGGAATGCAC |
| | | | | GGGACTGTGTACGCGAATTACGCGGTTGATAAGGCCGATTTGTTGC |
| | | | | TTGCTTTTGGGGTTAGGTTTGATGATCGAGTGACTGGTAAGCTCGA |
| | | | | GGCGTTTGCTAGCCGGGCTAAGATTGTGCACATCGATATCGATTCT |
| | | | | GCTGAAATCGGGAAGAATAAGCAACCTCATGTGTCGATTTGTGGTG |
| | | | | ATGTTAAAGTGGCATTACAGGGGTTGAATAAGATTTTGGAATCTAG |
| | | | | AAAAGGAAAGGTGAAATTGGATTTCTCTAATTGGAGGGAGGAGTT |
| | | | | GAATGAGCAGAAAAAGAAGTTTCCTTTGAGTTTTAAGACTTTCGGG |
| | | | | GATGCAATTCCTCCGCAATACGCCATTCAGGTTCTTGACGAGTTGAC |
| | | | | GAAGGGCGATGCGGTTGTAAGTACTGGTGTTGGGCAGCACCAAAT |
| | | | | GTGGGCTGCCCAATTCTATAAGTACCGAAATCCTCGCCAATGGCTG |
| | | | | ACCTCGGGTGGTTTGGGGGCTATGGGGTTTGGTCTACCAGCTGCTA |
| | | | | TTGGAGCTGCTGTTGCTCGACCAGATGCGGTGGTTGTAGACATTGA |
| | | | | TGGGGATGGGAGTTTTATCATGAATGTTCAAGAGTTGGCTACGATT |
| | | | | AGGGTAGAGAATCTCCCGGTTAAAATCATGCTCTTGAACAATCAAC |
| | | | | ATTTAGGTATGGTTGTTCAATGGGAAGATCGATTTTACAAAGCTAAC |
| | | | | CGGGCACATACATACCTCGGGAATCCTTCCAATTCTTCCGAAATCTT |
| | | | | CCCGGATATGCTCAAATTTGCTGAAGCATGTGATATACCAGCAGCC |
| | | | | CGTGTTACCAAGGTGAGCGATTTAAGGGCTGCAATTCAAACAATGT |
| | | | | TGGATACTCCAGGACCGTATCTGCTGGATGTAATCGTACCACATCA |
| | | | | GGAGCATGTGCTGCCTATGATCCCTAGCGGTGCCGCCTTCAAGGAC |
| | | | | ACCATAACAGAGGGTGATGGAAGAAGGGCTTATTAGTTGGTTGGA |
| | | | | GATCTTTATAGAGGAGAAGCTTTTTTGTATGTATGTTAGTAGTTCCA |
| | | | | TAAACTTCTATATTCTCTGGCCGTTCTCTCGTTTAGCTGTTTTTATGTT |
| | | | | AGTTTGTTGTTTTCATGTTGCTTGCTACTTTGAAAAACCCTTTTGTGT |
| | | | | TTTAGACCCATTAGCATGAATAATCTTCCTATATTATTGTATGGTTCG |
| | | | | ATACA |
| 5 | Amaranthus | cDNA | 2572 | CGCCTCCACTCTGGGTGATTCCCTTTCTCCATTCTCGCTTAGCTTTCC |
| | lividus | Contig | | TCTCACACAAATTACCTTTATTTCCAACCTTTCAAGCTTTCAACAATG |
| | | | | GCGTCCACTTCTTCAAACCCACCATTTTCCTCTTTTACTAAACCTAAC |
| | | | | AAAATCCCTAATCTGCAATCATCCATTTACGCTATCCCTTTTTCCAAT |
| | | | | TCTCTTAAACCCACTTCTTCTTCTTCAATCCTCCGCCGCCCTCTTCAAA |
| | | | | TCTCATCATCTTCTTCTCAATCACCTAAACCTAAACCTCCTTCCGCTAC |
| | | | | TATAACTCAATCACCTTCGTCTCTCACCGATGATAAACCCTCTTCTTT |
| | | | | TGTTTCCCGATTTAGCCCTGAAGAACCCAGAAAAGGTAGCGATGTT |
| | | | | CTCGTTGAAGCTCTTGAACGTGAAGGTGTTACCGATGTTTTTGCTTA |
| | | | | CCCTGGTGGAGCATCCATGGAAATTCATCAAGCTCTTACTCGTTCTA |
| | | | | ATATCATTAGAAATGTTCTTCCTCGACATGAACAAGGTGGGGTTTTC |
| | | | | GCTGCTGAAGGCTACGCTCGTGCTACTGGACGCGTTGGAGTTTGTA |
| | | | | TTGCCACTTCTGGTCCAGGTGCTACTAATCTTGTTTCTGGTCTTGCTG |
| | | | | ATGCACTTCTTGACTCAGTCCCTCTTGTCGCCATTACTGGGCAAGTT |
| | | | | CCCCGGCGTATGATTGGTACTGATGCTTTTCAAGAGACTCCAATTGT |
| | | | | TGAGGTAACTCGATCCATTACCAAGCATAATTATTTGGTGTTAGATG |
| | | | | TTGAGGATATTCCTAGAATTGTTAAGGAAGCTTTCTTTTTAGCTAATT |
| | | | | CTGGTAGACCTGGACCTGTTTTGATTGATATTCCTAAAGATATTCAG |
| | | | | CAACAATTAGTTGTTCCTAATTGGGAACAGCCCATTAAATTGGGTG |
| | | | | GGTATCTTTCTAGGTTGCCTAAACCCACTTATTCTGCTAATGAAGAG |
| | | | | GGACTTCTTGATCAAATTGTAAGGTTAGTGGGTGAGTCTAAGAGAC |
| | | | | CTGTGCTGTATACTGGAGGTGGGTGTTTGAATTCTAGTGAAGAATT |
| | | | | GAGGAAATTTGTCGAATTGACAGGTATTCCGGTGGCTAGTACTTTA |
| | | | | ATGGGGTTGGGGGCTTTCCCTTGTACTGATGATTTATCTCTTCATAT |
| | | | | GTTGGGAATGCACGGGACTGTGTACGCGAATTACGCGGTTGATAA |
| | | | | GGCCGATTTGTTGCTTGCTTTTGGGGTTAGGTTTGATGATCGAGTG |
| | | | | ACTGGTAAGCTCGAGGCGTTTGCTAGCCGGGCTAAGATTGTGCACA |
| | | | | TCGATATCGATTCTGCTGAAATCGGGAAGAATAAGCAACCTCATGT |
| | | | | GTCGATTTGTGGTGATGTTAAAGTGGCATTACAGGGGTTGAATAAG |
| | | | | ATTTTGGAATCTAGAAAAGGAAAGGTGAAATTGGATTTCTCTAATT |
| | | | | GGAGGGAGGAGTTGAATGAGCAGAAAAAGAAGTTTCCTTTGAGTT |
| | | | | TTAAGACTTTCGGGGATGCTATTCCTCCGCAATACGCCATTCAGGTT |
| | | | | CTTGACGAGTTGACGAAGGGCGATGCGGTTGTAAGTACTGGTGTTG |
| | | | | GGCAGCACCAAATGTGGGCTGCCCAATTCTATAAGTACCGAAATCC |
| | | | | TCGCCAATGGCTGACCTCGGGTGGTTTGGGGGCTATGGGGTTTGGT |
| | | | | CTACCAGCTGCTATTGGAGCTGCTGTTGCTCGACCAGATGCGGTGG |
| | | | | TTGTAGACATTGATGGGGATGGGAGTTTTATCATGAATGTTCAAGA |
| | | | | GTTGGCTACGATTAGGGTAGAGAATCTCCCGGTTAAAATCATGCTC |
| | | | | TTGAACAATCAACATTTAGGTATGGTTGTTCAATGGGAAGATCGATT |
| | | | | TTACAAAGCTAACCGGGCACATACATACCTCGGGAATCCTTCCAATT |
| | | | | CTTCCGAAATCTTCCCGGATATGCTCAAATTTGCTGAAGCATGTGAT |
| | | | | ATACCAGCAGCCCGTGTTACCAAGGTGAGCGATTTAAGGGCTGCAA |
| | | | | TTCAAACAATGTTGGATACTCCAGGACCGTATCTGCTGGATGTAATC |
| | | | | GTACCACATCAGGAGCATGTGCTGCCTATGATCCCTAGCGGTGCCG |
| | | | | CCTTCAAGGACACCATAACAGAGGGTGATGGAAGAAGGGCTTATT |
| | | | | AGTTGGTTGGAGATCTTTATAGAGGAGAAGCT |
| 6 | Amaranthus | cDNA | 2348 | TCCCTTTCTCCATTTTCGCTTAGCTTTCCTCTCACACAAATTACCTCCA |
| | palmeri | Contig | | TTTCCAACCCTCCAAGCTTTCAACAATGGCGTCCACTTCAACAAACC |
| | | | | CACCATTTTCCTCTTTTACTAAACCTAACAAAATCCCTAATCTGCAAT |
| | | | | CATCCATTTACGCTATCCCTTTTTCCAATTCTCTTAAACCCACTTCTTC |
| | | | | TTCTTCAATCCTCCGCCGCCCTCTTCAAATCTCATCATCTTCTTCTCAA |
| | | | | TCACCTAAACCTAAACCTCCTTCCGCTACTATAACTCAATCACCTTCA |
| | | | | TCTCTCACCGATGATAAACCCTCTTCTTTTGTTTCCCGATTTAGCCCT |
| | | | | GAAGAACCCAGAAAAGGTTGCGATGTTCTCGTTGAAGCTCTTGAAC |
| | | | | GTGAAGGTGTTACCGATGTTTTTGCTTACCCTGGTGGAGCATCCATG |
| | | | | GAAATCCATCAAGCTCTTACTCGTTCTAATATCATTAGAAATGTTCTT |
| | | | | CCTCGACATGAACAAGGTGGGGTTTTCGCTGCTGAAGGCTACGCTC |
| | | | | GTGCTACTGGACGCGTTGGAGTTTGTATTGCCACTTCTGGTCCAGGT |
| | | | | GCTACTAATCTTGTTTCTGGTCTTGCTGATGCACTTCTTGACTCAGTC |
| | | | | CCGCTTGTCGCCATTACTGGGCAAGTTCCCCGGCGTATGATTGGTAC |
| | | | | TGATGCTTTCCAAGAGACTCCAATTGTTGAGGTAACTCGATCCATTA |
| | | | | CTAAGCATAATTATTTGGTGTTAGATGTTGAGGATATTCCTAGAATT |
| | | | | GTTAAGGAAGCTTTCTTTTTAGCTAATTCTGGTAGACCTGGACCTGT |
| | | | | TTTGATTGATATTCCTAAAGATATTCAGCAACAATTAGTTGTTCCTAA |
| | | | | TTGGGAACAGCCCATTAAATTGGGTGGGTATCTTTCTAGGTTGCCTA |
| | | | | AACCCACTTATTCTGCTAATGAAGAGGGACTTCTTGATCAAATTGTA |
| | | | | AGGTTAGTGGGTGAGTCTAAGAGACCTGTGCTGTATACTGGAGGT |
| | | | | GGGTGTTTGAATTCTAGTGAAGAATTGAGGAAATTTGTCGAATTGA |
| | | | | CAGGGATTCCGGTGGCTAGTACTTTAATGGGGTTGGGGGCTTTCCC |
| | | | | TTGTACTGATGATTTATCACTTCATATGTTGGGAATGCACGGGACTG |
| | | | | TGTATGCGAATTACGCGGTTGATAAGGCCGATTTGTTGCTTGCTTTC |
| | | | | GGGGTTAGGTTTGATGATCGAGTGACTGGTAAGCTCGAGGCGTTT |
| | | | | GCTAGCCGGGCTAAGATTGTGCACATCGATATCGATTCTGCTGAAA |
| | | | | TCGGGAAGAATAAGCAACCTCATGTGTCGATTTGTGGTGATGTTAA |
| | | | | AGTGGCATTACAGGGGTTGAATAAGATTTTGGAATCTAGAAAAGG |
| | | | | AAAGGTGAAATTGGATTTCTCTAATTGGAGGGAGGAGTTGAATGA |
| | | | | GCAGAAAAAGAAGTTTCCTTTGAGTTTTAAGACTTTCGGGGATGCA |
| | | | | ATTCCTCCGCAATACGCCATTCAGGTTCTTGACGAGTTGACGAAGG |
| | | | | GTGATGCGGTTGTAAGTACCGGTGTTGGGCAGCACCAAATGTGGG |
| | | | | CTGCCCAATTCTATAAGTACCGAAATCCTCGCCAATGGCTGACCTCG |
| | | | | GGTGGTTTGGGGGCTATGGGGTTTGGTCTACCAGCTGCAATTGGA |
| | | | | GCTGCTGTTGCTCGACCAGATGCGGTGGTTGTAGACATTGATGGGG |
| | | | | ATGGGAGTTTTATCATGAATGTTCAAGAGTTGGCTACGATTAGGGT |
| | | | | GGAGAATCTCCCGGTTAAAATCATGCTCTTGAACAATCAACATTTAG |
| | | | | GTATGGTTGTTCAATGGGAAGATCGATTTTACAAAGCTAACCGGGC |
| | | | | ACATACATACCTCGGGAATCCTTCCAATTCTTCCGAAATCTTCCCGG |
| | | | | ATATGCTCAAATTCGCTGAAGCATGTGATATACCAGCAGCTCGTGTT |
| | | | | ACCAAGGTGAGCGATTTAAGGGCTGCAATTCAAACAATGTTGGATA |
| | | | | CTCCAGGACCGTATCTGCTGGATGTAATCGTACCACATCAGGAGCA |
| | | | | TGTGCTGCCTATGATCCCTAGCGGTGCCGCCTTCAAGGACACCATCA |
| | | | | CAGAGGGTGATGGAAGAAGGGCTTATTAGTTGGTTGGAGATCTTTA |
| | | | | TAGAGGAGAAGCTTTTTTGTATGTATGTTAGTATTTCCATAAACTTCT |
| | | | | ATATTCTCTGGCCGGCCTCTCGTTTAGCTGTTTTTATGTTAGTTTGTT |
| | | | | GTTTTCATGTTGCTTGCTACTTTGAAAAACCCTTGTGTTTTAGACCCA |
| | | | | TTAGCATGAATAATCATCCTATATTATTGTATGGTTTGATACACGCTA |
| | | | | GTTGTTTCTTTTTATCATCGAGATAAAGACAACTACTAATCGGTGTA |
| | | | | ACCACTGT |
| 7 | Amaranthus | gDNA | 6061 | TGATCATGAAATTAGTTTGAGGAATCGTTTTTTCGAGTACTGAAGAC |
| | palmeri | Contig | | TAGAAAGGAACTTGAGACATCAGTTTGGGAATTCGATGGAGTAGC |
| | | | | GGATCGTTCTTCACTTTTTGCTCGGGAGGTATCAATGGCGTAAATTT |
| | | | | TTTTTGGAGACTGACTCAAATGGGATTCTTTGTGTAGTGTACAGCTT |
| | | | | CACTTACCACAGGAACACAGGTGTTTTTTGCTATATAGGTACAGAAA |
| | | | | CTCGGAAAAGTCCACATTTACTCTATAACTTCATTTACGGTCTAAAA |
| | | | | GTAGTTGAGAAGGTAAAATTTAGGATGCAATTTTTTGAAAGCTTTCT |
| | | | | ATGATAGCAAATTCTTACTTTTTATTTCTACTGGTAATTGTCATTTTG |
| | | | | TCAATGTCCTTTGCAAAGACTAATTTTACAGACTTGTTATTTTCGTTT |
| | | | | AAAATGTTTCGTATGATACCGAACCAGAAAGAGAAACAAGAAATTC |
| | | | | GGGCGAATTTTTCTCGTCATATGTTAGATCGAAACCATATGATCAGC |
| | | | | TTGATCTTGGTTGTACTGGTGTATTGATTTTCATGGTAAATGTCAAT |
| | | | | ATTTTCATGGTAAACAACTTGGGTGCCAATTTTGTTAGGGTTAGATT |
| | | | | TGGTGCTGCTTTTGAATCAGAGATGAAATCAGCCGAGAAATGAAGA |
| | | | | TAAATTGGGGTGATTTTGCAAAATAGTTTGTCTGTTAATTTTAACTTA |
| | | | | TATTTGTTACATTCTTTGTTACTTGCACCATTGTTGACTTTGGTACTAT |
| | | | | TCATCAAGCAAATCGACTTTCATATTGTCGGTAATCTATAAAATAAA |
| | | | | ATATAGTTTTCTGAGATCTTGTTGAATCGTCTCGATTCAACGGTTATT |
| | | | | AATTTTTAGTTTTCATAATTTTTTGATATGTATAATAAAAGATATTAA |
| | | | | CAATTGATATCCTGTATTTGGGGTACGAAAAAGCAAATAGTGCAAG |
| | | | | TAAAAAAGAACGGAGGAAGTACATGTACAATTATCACTCAACTATT |
| | | | | AGATAAATTAGTTGGTTGAGATAAATCGTTAGAACTTAGAAATCAC |
| | | | | CAAATCTATAATAAGCTTTTCGCATTAGCGTGTTTGATATCAGCTGC |
| | | | | TTAGTTGATGAAATTAGATGGTTAAATCACTCGCTTGAAGCTATCAC |
| | | | | TTATCAACCGTTTGTCAAAGATCCTTGCATTTTAATTCATTGTGACTT |
| | | | | GTGAGATAGATCTATATGAAGAATTTAAATTTTAACAATAAGCGTTT |
| | | | | TAATATTTAAAGCTGGTGTTTTGATAAATTTAATTAAACTATTTAGAT |
| | | | | AAGTTCGGACTGTCTCTTCATGAGAGATGAGACTGTATCAATCAGCT |
| | | | | AATTTATAATCACCTAAATATCTCCTTATCATCATAATACATCCACCT |
| | | | | CATAAAATCTATAACCAGAATCTAAAAGAGGGATGGAAGACGACA |
| | | | | AGTCAACCCCTCACCTAATCATGTCTTTGATGTTAATAGCATAAAAG |
| | | | | CACATGAGTGATGAGAACAAACACATCAACCCTAATCTTATCCAAG |
| | | | | AAAATACCCCATTAGGCATTAAGGTCCTTTTCTTTTGTTTTCTTTTGC |
| | | | | CCTAAAAAGCAAAAGTGGAAAGAAACCCAAAAAAATCAATTTCACG |
| | | | | TTGCAAATTGTCAAATTTTTTTATAAAAAAAAATGAAAAATCTTTATA |
| | | | | CCACAAAAACAAAAAGCAACAATGAAAATTTAGAATCATACTCCCG |
| | | | | TTTCAATTTACTTGCAACAGTTACGCAGTTTAATACACTAATTCAATC |
| | | | | CTTAATATCTATAATTATAAATAATAAAAAATTATAAAAATTTAATAT |
| | | | | TAATAATCTTTGCAATGAGATGAATCAAACAAGATTTCACTAATAAA |
| | | | | TAATGTCATTTTTTTATATGTTGCAACTGATATGGAACAGAGAAAGT |
| | | | | ATCAATCTTGATATTCAAGGGTATTTAAGTAATTACAGAACAACCAT |
| | | | | TGTTATTTGTTAAGCGCCTCCACTCATTTCTTCTTCCTTCTCAGTTATT |
| | | | | CCATTTCTCCATTTTCGCTTAGCTTTCCTCTCACACAAATTACCTCCAT |
| | | | | TTCCAACCTTTCAAGCTTTCAACAATGGCGTCCACTTCAACAAACCC |
| | | | | ACCATTTTCCTCTTTTACTAAACCTAACAAAATCCCTAATCTGCAATC |
| | | | | ATCCATTTACGCTATCCCTTTTTCCAATTCTCTTAAACCCACTTCTTCT |
| | | | | TCTTCAATCCTCCGCCGCCCTCTTCAAATCTCATCATCTTCTTCTCAAT |
| | | | | CACCTAAACCTAAACCTCCTTCCGCTACTATAACTCAATCACCTTCAT |
| | | | | CTCTCACCGATGATAAACCCTCTTCTTTTGTTTCCCGATTTAGCCCTG |
| | | | | AAGAACCCAGAAAAGGTTGCGATGTTCTCGTTGAAGCTCTTGAACG |
| | | | | TGAAGGTGTTACCGATGTTTTTGCTTACCCTGGTGGAGCATCCATGG |
| | | | | AAATCCATCAAGCTCTTACTCGTTCTAATATCATTAGAAATGTTCTTC |
| | | | | CTCGACATGAACAAGGTGGGGTTTTCGCTGCTGAAGGCTACGCTCG |
| | | | | TGCTACTGGACGCGTTGGAGTTTGTATTGCCACTTCTGGTCCAGGTG |
| | | | | CTACTAATCTTGTTTCTGGTCTTGCTGATGCACTTCTTGACTCAGTCC |
| | | | | CGCTTGTCGCCATTACTGGGCAAGTTCCCCGGCGTATGATTGGTACT |
| | | | | GATGCTTTCCAAGAGACTCCAATTGTTGAGGTAACTCGATCCATTAC |
| | | | | TAAGCATAATTATTTGGTGTTAGATGTTGAGGATATTCCTAGAATTG |
| | | | | TTAAGGAAGCTTTCTTTTTAGCTAATTCTGGTAGACCTGGACCTGTT |
| | | | | TTGATTGATATTCCTAAAGATATTCAGCAACAATTAGTTGTTCCTAAT |
| | | | | TGGGAACAGTCCATTAAATTGGGTGGGTATCTTTCTAGGTTGCCTAA |
| | | | | ACCCACTTATTCTGCTAATGAAGAGGGACTTCTTGATCAAATTGTAA |
| | | | | GGTTAGTGGGTGAGTCTAAGAGACCTGTGCTGTATACTGGAGGTG |
| | | | | GGTGTTTGAATTCTAGTGAAGAATTGAGGAAATTTGTCGAATTGAC |
| | | | | AGGGATTCCGGTGGCTAGTACTTTAATGGGTTTGGGGGCTTTCCCT |
| | | | | TGTACTGATGATTTATCACTTCATATGTTGGGAATGCACGGGACTGT |
| | | | | GTATGCGAATTACGCGGTTGATAAGGCCGATTTGTTGCTTGCTTTCG |
| | | | | GGGTTAGGTTTGATGATCGAGTGACTGGTAAGCTCGAGGCGTTTGC |
| | | | | TAGCCGGGCTAAGATTGTGCACATCGATATCGATTCTGCTGAAATC |
| | | | | GGGAAGAATAAGCAACCTCATGTGTCGATTTGTGGTGATGTTAAAG |
| | | | | TGGCATTACAGGGGTTGAATAAGATTTTGGAATCTAGAAAAGGAAA |
| | | | | GGTGAAATTGGATTTCTCTAATTGGAGGGAGGAGTTGAATGAGCA |
| | | | | GAAAAAGAAGTTTCCTTTGAGTTTTAAGACTTTCGGGGATGCAATTC |
| | | | | CTCCGCAATACGCCATTCAGGTTCTTGACGAGTTGACAAAGGGTGA |
| | | | | TGCGGTTGTAAGTACCGGTGTTGGGCAGCACCAAATGTGGGCTGCC |
| | | | | CAATTCTATAAGTACCGAAATCCTCGTCAATGGCTGACCTCGGGTG |
| | | | | GTTTGGGGGCTATGGGGTTTGGTCTACCAGCTGCTATTGGAGCTGC |
| | | | | TGTTGCTCGACCAGATGCGGTGGTTGTAGACATTGATGGGGATGG |
| | | | | GAGTTTTATCATGAATGTTCAAGAGTTGGCTACGATTAGGGTGGAG |
| | | | | AATCTCCCGGTTAAAATCATGCTCTTGAACAATCAACATTTAGGTAT |
| | | | | GGTTGTTCAATGGGAAGATCGATTTTACAAAGCTAACCGGGCACAT |
| | | | | ACATACCTCGGGAATCCTTCCAATTCTTCCGAAATCTTCCCGGATAT |
| | | | | GCTCAAATTTGCTGAAGCATGTGATATACCAGCAGCCCGTGTTACCA |
| | | | | AGGTGAGCGATTTAAGGGCTGCAATTCAAACAATGTTGGATACTCC |
| | | | | AGGACCGTATCTGCTGGATGTAATCGTACCACATCAGGAGCATGTG |
| | | | | CTGCCTATGATCCCTAGCGGCGCCGCCTTCAAGGACACCATCACTGA |
| | | | | GGGTGATGGAAGAAGGGCTTATTAGTTGGTTGGAGATCTTTATAGA |
| | | | | GGAGAAGCTTTTTTGTATGTATGTTAGTATTTCCATAAACTTCTATAT |
| | | | | TCTCTGGCCGGCCTCTCGTTTAGCTGTTTTTATGTTAGTTTGTTGTTT |
| | | | | TCATGTTGCTTGCTACTTTGAAAAACCCTTTTGTGTTTTAGACCCATT |
| | | | | AGCATGAATAATCATCCTATATTATTGTATGGTTTGATACACGCTAG |
| | | | | TTGTTTCTTTTTATCATCGAGATAAAGACAACTACTAATCGGTGTAA |
| | | | | CCATGTTTGTCTCACATTGCTCGACGTTTTGTTCTCCTTTGATGAATT |
| | | | | TGATATTAGGAAGTTTGTTTGATACTAGTGAATCCTCATGTCCTATC |
| | | | | CTGTTTTCTCGTCTTAGCGAACATTGGTATTGTATCTCTTGGTGGGT |
| | | | | ATAGGAGTATTCGATTCGTTGTTATGTTCGACTCCTTCAGTGCTTTTG |
| | | | | GTGGGTTTTTCAAAGAAGGAAAAGGAAGCAGAAATGGCCTTAGAA |
| | | | | TGTAAAGTGGTCTTTTTCTGTAAGTGGGTATTGCCTTTCATCAGCCA |
| | | | | TGTTTTTATCTTTATATTTTGTGGTCTTTGAGGCTTTATGTTTAGTGTT |
| | | | | TGGTGTCTTTTCGTCGTATTCTGTGATCAGTTCTGAAATGTGGATCG |
| | | | | TAAAGACATAGATGATGAGGAAGAGGGTCGGGATGAGAGCAGCG |
| | | | | TTTTACTCGAAAAGCGATCCTAGTAAGGACCGAACCGAGTCAACAT |
| | | | | TTCGAGATGATATGTGTTCGAGTTTGACATGAAAATCTATCTGCCTG |
| | | | | CTCAGTGCTCACAATGGTAAATTGTACTGTAAATGATTGTTATGTCA |
| | | | | TGTTGGTTTTTCATCTGGAAATGTGTATCTGTAATGGTTCTTGTCAGT |
| | | | | TGATTGTATCGGTGTTCATTCGGGTTGATTTTGGGTTAGGTTTTCAA |
| | | | | GACTGTTTGAAATCAATTTATGTCATTGTGTCTATATTGTTTGTTACA |
| | | | | TAACTGCAAATCATTTTTGAAGTCGTATCAAATCGGATTCAATTATA |
| | | | | AGGTTGGGTGAATTTTGAGTCATTGGGTCTGAATTAAACACCTATCC |
| | | | | CAAGGATTATACATTTGTTGCAATTTTGCTCTTTTCATTCTTAAATTT |
| | | | | GGCTTCATCACGGTGTTTTGTTTTATAAGATTCTATTCGAATCTAAAG |
| | | | | CCGAACACACTTGATCATTAACATATGACTAATTTAACATAATTGGC |
| | | | | CGGCTAATTAGCCATATAAATTCAAGGTTGCTTAAAATTGTTGAAGA |
| | | | | ATAGCCCTAAAGACGTATAATCACTTTTAATATTTTTTAATTTTTTAA |
| | | | | CGTGATTTGATTTGATATCTTTTAATATACTATTAGGCTCATTGTATG |
| | | | | CTACAAATTTGAGGGGAAAACTATGAACAACAATTCATATTAATAT |
| | | | | GTTTTTTAACAAACAAGAAAAATAGAAAGTTAAAAGACAATGATTC |
| | | | | ATCAAATAAATCATGTGTACTTTTTATTCGTTTGTAGTTGCATATGAC |
| | | | | GTTACAACCATCGAAAACAATCATTTTATTAGTTTTATCATTGATTGT |
| | | | | TTTTGATGGTTGTAACGTCATATGCAACCATAAACGTATAAAAAGTG |
| | | | | ACTCAATTTCTAGAGGCTAAAACAATTGGGTTGCCTAACAAGTGTTG |
| | | | | ATTTAATATTAGGTTGTTTGTGCTTACTTAATAAATCTCTACACTAGT |
| | | | | CTTAAAATTGTAATCTAAATTGTGCTACAAAAATAGGTAATTAATTA |
| | | | | GAAAAGTCGATTAAATCAATCTTTTAGGGTATATTAGCAATTATTAC |
| | | | | TAATGATTATAAGTAACCGAGAGGTCTTGTCATTTAGGATTAATTGA |
| | | | | TATTAGGTCTTTTGAGAGCCGTCTTTACAAAATACGGTCTCAAGTAA |
| | | | | GAATTTATGAATTTTTTTTATTCTTACATGACTCATGGAAAGATTCCT |
| | | | | AGTTGTTCCAAAGCCCAATCATCTATTCACAATTTATTTAAATTCGAC |
| | | | | CCAAACTAATTTATTCAACTCAACCTAAAATGATTCTTGTTC |
| 8 | Amaranthus | gDNA | 5410 | TGATCATGAAATTAGTTTGAGGAATCGTTTTTTCGAGTACTGAAGAC |
| | palmeri | Contig | | TAGAAAGGAACTTGAGACATCAGTTTGGGAATTCGATGGAGTAGC |
| | | | | GGATCGTTCTTCACTTTTTGCTCGGGAGGTATCAATGGCGTAAATTT |
| | | | | TTTTTGGAGACTGACTCAAATGGGATTCTTTGTGTAGTGTACAGCTT |
| | | | | CACTTACCACAGGAACACAGGTGTTTTTTGCTATATAGGTACAGAAA |
| | | | | CTCGGAAAAGTCCACATTTACTCTATAACTTCATTTACGGTCTAAAA |
| | | | | GTAGTTGAGAAGGTAAAATTTAGGATGCAATTTTTTGAAAGCTTTCT |
| | | | | ATGATAGCAAATTCTTACTTTTTATTTCTACTGGTAATTGTCATTTTG |
| | | | | TCAATGTCCTTTGCAAAGACTAATTTTACAGACTTGTTATTTTCGTTT |
| | | | | AAAATGTTTCGTATGATACCGAACCAGAAAGAGAAACAAGAAATTC |
| | | | | GGGCGAATTTTTCTCGTCATATGTTAGATCGAAACCATATGATCAGC |
| | | | | TTGATCTTGGTTGTACTGGTGTATTGATTTTCATGGTAAATGTCAAT |
| | | | | ATTTTCATGGTAAACAACTTGGGTGCCAATTTTGTTAGGGTTAGATT |
| | | | | TGGTGCTGCTTTTGAATCAGAGATGAAATCAGCCGAGAAATGAAGA |
| | | | | TAAATTGGGGTGATTTTGCAAAATAGTTTGTCTGTTAATTTTAACTTA |
| | | | | TATTTGTTACATTCTTTGTTACTTGCACCATTGTTGACTTTGGTACTAT |
| | | | | TCATCAAGCAAATCGACTTTCATATTGTCGGTAATCTATAAAATAAA |
| | | | | ATATAGTTTTCTGAGATCTTGTTGAATCGTCTCGATTCAACGGTTATT |
| | | | | AATTTTTAGTTTTCATAATTTTTTGATATGTATAATAAAAGATATTAA |
| | | | | CAATTGATATCCTGTATTTGGGGTACGAAAAAGCAAATAGTGCAAG |
| | | | | TAAAAAAGAACGGAGGAAGTACATGTACAATTATCACTCAACTATT |
| | | | | AGATAAATTAGTTGGTTGAGATAAATCGTTAGAACTTAGAAATCAC |
| | | | | CAAATCTATAATAAGCTTTTCGCATTAGCGTGTTTGATATCAGCTGC |
| | | | | TTAGTTGATGAAATTAGATGGTTAAATCACTCGCTTGAAGCTATCAC |
| | | | | TTATCAACCGTTTGTCAAAGATCCTTGCATTTTAATTCATTGTGACTT |
| | | | | GTGAGATAGATCTATATGAAGAATTTAAATTTTAACAATAAGCGTTT |
| | | | | TAATATTTAAAGCTGGTGTTTTGATAAATTTAATTAAACTATTTAGAT |
| | | | | AAGTTCGGACTGTCTCTTCATGAGAGATGAGACTGTATCAATCAGCT |
| | | | | AATTTATAATCACCTAAATATCTCCTTATCATCATAATACATCCACCT |
| | | | | CATAAAATCTATAACCAGAATCTAAAAGAGGGATGGAAGACGACA |
| | | | | AGTCAACCCCTCACCTAATCATGTCTTTGATGTTAATAGCATAAAAG |
| | | | | CACATGAGTGATGAGAACAAACACATCAACCCTAATCTTATCCAAG |
| | | | | AAAATACCCCATTAGGCATTAAGGTCCTTTTCTTTTGTTTTCTTTTGC |
| | | | | CCTAAAAAGCAAAAGTGGAAAGAAACCCAAAAAAATCAATTTCACG |
| | | | | TTGCAAATTGTCAAATTTTTTTATAAAAAAAAATGAAAAATCTTTATA |
| | | | | CCACAAAAACAAAAAGCAACAATGAAAATTTAGAATCATACTCCCG |
| | | | | TTTCAATTTACTTGCAACAGTTACGCAGTTTAATACACTAATTCAATC |
| | | | | CTTAATATCTATAATTATAAATAATAAAAAATTATAAAAATTTAATAT |
| | | | | TAATAATCTTTGCAATGAGATGAATCAAACAAGATTTCACTAATAAA |
| | | | | TAATGTCATTTTTTTATATGTTGCAACTGATATGGAACAGAGAAAGT |
| | | | | ATCAATCTTGATATTCAAGGGTATTTAAGTAATTACAGAACAACCAT |
| | | | | TGTTATTTGTTAAGCGCCTCCACTCATTTCTTCTTCCTTCTCAGTTATT |
| | | | | CCATTTCTCCATTTTCGCTTAGCTTTCCTCTCACACAAATTACCTCCAT |
| | | | | TTCCAACCTTTCAAGCTTTCAACAATGGCGTCCACTTCAACAAACCC |
| | | | | ACCATTTTCCTCTTTTACTAAACCTAACAAAATCCCTAATCTGCAATC |
| | | | | ATCCATTTACGCTATCCCTTTTTCCAATTCTCTTAAACCCACTTCTTCT |
| | | | | TCTTCAATCCTCCGCCGCCCTCTTCAAATCTCATCATCTTCTTCTCAAT |
| | | | | CACCTAAACCTAAACCTCCTTCCGCTACTATAACTCAATCACCTTCAT |
| | | | | CTCTCACCGATGATAAACCCTCTTCTTTTGTTTCCCGATTTAGCCCTG |
| | | | | AAGAACCCAGAAAAGGTTGCGATGTTCTCGTTGAAGCTCTTGAACG |
| | | | | TGAAGGTGTTACCGATGTTTTTGCTTACCCTGGTGGAGCATCCATGG |
| | | | | AAATCCATCAAGCTCTTACTCGTTCTAATATCATTAGAAATGTTCTTC |
| | | | | CTCGACATGAACAAGGTGGGGTTTTCGCTGCTGAAGGCTACGCTCG |
| | | | | TGCTACTGGACGCGTTGGAGTTTGTATTGCCACTTCTGGTCCGGGT |
| | | | | GCTACTAATCTTGTCTCTGGTCTTGCTGATGCGCTTCTTGACTCAGTC |
| | | | | CCGCTCGTCGCCATTACTGGGCAAGTTCCCCGGCGTATGATTGGTAC |
| | | | | TGATGCTTTTCAAGAGACTCCAATTGTTGAGGTAACTCGATCCATTA |
| | | | | CTAAGCATAATTATTTGGTGTTAGATGTTGAGGATATTCCTAGAATT |
| | | | | GTTAAGGAAGCTTTCTTTTTAGCTAATTCTGGTAGACCTGGACCTGT |
| | | | | TTTGATTGATATTCCTAAAGATATTCAGCAACAATTAGTTGTTCCTAA |
| | | | | TTGGGAACAGCCCATTAAATTGGGTGGGTATCTTTCTAGGTTGCCTA |
| | | | | AACCCACTTATTCTGCTAATGAAGAGGGACTTCTTGATCAAATTGTA |
| | | | | AGGTTAGTGGGTGAGTCTAAGAGACCTGTGCTGTATACTGGAGGT |
| | | | | GGGTGTTTGAATTCTAGTGAAGAATTGAGGAAATTTGTCGAATTGA |
| | | | | CAGGGATTCCGGTGGCTAGTACTTTAATGGGTTTGGGGGCTTTCCC |
| | | | | TTGTACTGATGATTTATCACTTCATATGTTGGGAATGCACGGGACTG |
| | | | | TGTATGCGAATTACGCGGTTGATAAGGCCGATTTGTTGCTTGCTTTC |
| | | | | GGGGTTAGGTTTGATGATCGAGTGACTGGTAAGCTCGAGGCGTTT |
| | | | | GCTAGCCGGGCTAAGATTGTGCACATCGATATCGATTCTGCTGAAA |
| | | | | TCGGGAAGAATAAGCAACCTCATGTGTCGATTTGTGGTGATGTTAA |
| | | | | AGTGGCATTACAGGGGTTGAATAAGATTTTGGAATCTAGAAAAGG |
| | | | | AAAGGTGAAATTGGATTTCTCTAATTGGAGGGAGGAGTTGAATGA |
| | | | | GCAGAAAAAGAAGTTTCCTTTGAGTTTTAAGACTTTCGGGGATGCA |
| | | | | ATTCCTCCGCAATACGCCATTCAGGTTCTTGACGAGTTGACAAAGG |
| | | | | GTGATGCGGTTGTAAGTACCGGTGTTGGGCAGCACCAAATGTGGG |
| | | | | CTGCCCAATTCTATAAGTACCGAAATCCTCGCCAATGGCTGACCTCG |
| | | | | GGTGGTTTGGGGGCTATGGGGTTTGGTCTACCAGCTGCTATTGGAG |
| | | | | CTGCTGTTGCTCGACCAGATGCGGTGGTTGTAGACATTGATGGGGA |
| | | | | TGGGAGTTTTATCATGAATGTTCAAGAGTTGGCTACGATTAGGGTG |
| | | | | GAGAATCTCCCGGTTAAAATCATGCTCTTGAACAATCAACATTTAGG |
| | | | | TATGGTTGTTCAATGGGAAGATCGATTTTACAAAGCTAACCGGGCA |
| | | | | CATACATACCTCGGGAATCCTTCCAATTCTTCCGAAATCTTCCCGGAT |
| | | | | ATGCTCAAATTCGCTGAAGCATGTGATATACCAGCAGCTCGTGTTAC |
| | | | | CAAGGTGAGCGATTTAAGGGCTGCAATTCAAACAATGTTGGATACT |
| | | | | CCAGGACCGTATCTGCTGGATGTAATCGTACCACATCAGGAGCATG |
| | | | | TGCTGCCTATGATCCCTAGCGGTGCCGCCTTCAAGGACACCATCACA |
| | | | | GAGGGTGATGGAAGAAGGGCTTATTAGTTGGTTGGAGATCTTTATA |
| | | | | GAGGAGAAGCTTTTTTGTATGTATGTTAGTATTTCCATAAACTTCTAT |
| | | | | ATTCTCTGGCCGGCCTCTCGTTTAGCTGTTTTTATGTTAGTTTGTTGT |
| | | | | TTTCATGTTAGTTTGTTGTTTTCATGTTGCTTGCTACTTTGAAAAACC |
| | | | | CTTTTGTGTTTTAGACCCATTAGCATGAATAATCATCCTATATTATTG |
| | | | | TATGGTTCGATACACGCTAGTTGTTTCTTTTTATTATCGAGTTAAAGA |
| | | | | CAACTACTAATCGGTGTAACCAAGTTTGTCTCACATTGCTCGACGTT |
| | | | | TTGTTCTCCTTTGATGAATTTGATATTAGGAAGTTTGTTTGATACTAG |
| | | | | TGAATCCTCATGTCCTATCCTGTTTTCTCGTCTTAGCGAACATTGGTA |
| | | | | TTGTATCTCTTGGTGGGTATAGGAGTATTCGATTCGTTGTTATGTTC |
| | | | | GACTCCTTCAGTGCTTTTGGTGGGTTTTTCAAAGAAGGAAAAGGAA |
| | | | | GCAGAAATGGCCTTAGAATGTAAAGTGGTCTTTTTCTGTAAGTGGG |
| | | | | TATTGCCTTTCATCAGCCATGTTTTTATCTTTATATTTTGTGGTCTTTG |
| | | | | AGGCTTTATGTTTAGTGTTTGGTGTCTTTTCGTCGTATTCTGTGATCA |
| | | | | GTTCTGAAATGTGGATCGTAAAGACATAGATGATGAGGAAGAGGG |
| | | | | TCGGGATGAGAGCAGCGTTTTACTCGAAAAGCGATCCTAGTAAGGA |
| | | | | CCGAACCGAGTCAACATTTCGAGATGATATGTGTTCGAGTTTGACAT |
| | | | | GAAAATCTATCTGCCTGCTCAGTGCTCACAATGGTAAATTGTACTGT |
| | | | | AAATGATTGTTATGTCATGTTGGTTTTTCATCTGGAAATGTGTATCT |
| | | | | GTAATGGTTCTTGTCAGTTGATTGTATCGGTGTTCATTCGGGTTGAT |
| | | | | TTTGGGTTAGGTTTTCAAGACTGTTTGAAATCAATTTATGTCATTGT |
| | | | | GTCTATATTGTTTGTTACATAACTGCAAATCATTTTTGAAGTCGTATC |
| | | | | AAATCGGATTCAATTATAAGGTTGGGTGAATTTTGAGTCATTGGGT |
| | | | | CTGAATTAAACACCTATCCCAAGGATTATACATTTGTTGCAATTTTGC |
| | | | | TCTTTTCATTCTTAAATTTGGCTTCATCACGGTGTTTTGTTTTATAAG |
| | | | | ATTCTATTCGAATCTAAAGCCGAACACACTTGATCATTAACATATGA |
| | | | | CTAATTTAACATAATTCTCCGGCTAATTAGCCATATAAATTCAAGGTT |
| | | | | GCTTAAAATTGTTGAAGAATAGCCCTAAAGACGTATAATCACTTTTA |
| | | | | ATATTTTTTAATTTTTTAACGTGATTTGATTTGATATCTTTTAATATAC |
| | | | | TATT |
| 9 | Amaranthus | cDNA | 3817 | AACAGGGAAGGCCATTATGTTTCAATCACCAAAATTGGCCCAAATC |
| | rudis | Contig | | TGAAAGAGCATAGGACACGATGGTAGTAAAACCAGGTTTATAATCA |
| | | | | GTCAAGAAATCTAAAACTTCCAACAATCATACACAAAACCAATGGA |
| | | | | ATCAATATTGCAGCCAATTGCAGAGCGATATGCACAATCATCTTCAA |
| | | | | AAAAGAAGCTACCCGGCCCTGAACACCACTGCAAACTGTCCTTCACT |
| | | | | GCACAATCATCTTCGATGCACATTCCAACCTCATCCGCCATTCTTCAC |
| | | | | ATTCATAGTTACCCGACCAGCCTCTAGTTAAAATCCTGATGAGTAAT |
| | | | | ACTGTCAAACCAAATCCACGTGAAAAAGATGAAAGACCACAATTGA |
| | | | | TTTGTGGTAAAATAAATAATTGGGCCCTGTTCTTTATCTTGCAAATA |
| | | | | CAACCTTGTCTTCTGAAAAAGTAGGCACAAACTCCCTAGAGTTCGG |
| | | | | AAGAGGAACTTCCTTGAACCAAGGATGATCAAGAGCCTCTTCTGCG |
| | | | | GTTATCCTCTTCTCAGGGTCATAGGTTAATAGACGGCTCAACAAATC |
| | | | | AAGCCCCGTGTCAGAAAGTGCAGGAGCACCTGTAAAAGATGTGCG |
| | | | | AGGAAACTTCTTATGCAATTGATTGTACGGTTGGCGAATAAAGTTG |
| | | | | AACTTGTGCCCAGGCAATTGAGAAAATCCTGGCCAAAGTTTGTCATT |
| | | | | AGGTGTGCCCAATGTTGCAAACATCTTGTTAAGCTGCTCAATCTCAG |
| | | | | ATTTCCCATCAAATAAGGGTTTATTGGCCAATAGTTCAGCCATGATG |
| | | | | CAACCCACAGACCACATATCCACAGCAGTTGAGTACTCTGTAGCTCC |
| | | | | AAGAAGAAGTTCAGGAGCCCTGTACCAAAGAGTAACCACCAAAGC |
| | | | | AGTATAAGGCTTCAACGGGCTACCATACTGACGTGCCATACCGAAG |
| | | | | TCGCATATCTTTAGCTCTCCCTTGTTATTTACCAAAAGATTTGAAGTC |
| | | | | TTCAAATCTCTATGTAGCACCCAATTATGATGAAGATACTTCACACC |
| | | | | CTCCAAAAGTTGAAGCATTAAACACTTAACTTCACTATATCTGAAAG |
| | | | | GCTGCTTCCTAGTCTGCATTAAAGCCTTAAGATCATGCTCTACATGA |
| | | | | TCCATCACCATGTAAATGCTATCAAAGCCATCCGGGCTATCATCAAC |
| | | | | CACTACTTCTCGAACATTCACAATTGAAGGATGATTCAAGGACACAA |
| | | | | GAGTATTGATTTCCCTCAAGTAATAAACAGGAAACCCCTCTCTTTGG |
| | | | | TCACCCAACTTCATCTTCTTTAAGGCAACAATCTCACCACTTTCTTCA |
| | | | | TCTCTTGCTTTGTACACAATACCATAACTTCCTTCACTAATTTTATTAA |
| | | | | GCTTCTTATACTTGAACACACTTCTGCATCCCTGAAGCATATTTCTAT |
| | | | | TTCCTCTCCCGGAAACCCCGGGCTCTCCACCGTCATCACTCGCAGCA |
| | | | | CAATCCTCACCATCACTCGCATCAACCTCCATACAGTCATCTCTCTTC |
| | | | | AAACCCACCATTTTCTATTTTACTATACCTTACAAAATCCCTAATCTG |
| | | | | CAATCATCCATTTACGCTATCCCTTTTTCCGATTCTCTTAAACCCACTT |
| | | | | CTTCTTCTTCAATCCTCCGCCGCCCTCTTCAAATCTCATCATCTTCTTC |
| | | | | TCAATCACCTAAACCTAAACCTCCTTCCGCTACTATAACTCAATCACC |
| | | | | TTCGTCTCTCACCTATGATAAACCCTCTTCTTTTGTTTCCCGATTTAGC |
| | | | | CCTGAAGAACCCAGAAAAGGTTGCGATGTTCTCGTTGAAGCTCTTG |
| | | | | AACGTGAAGGTGTTACCGATGTTTTTGCTTACCCTGGTGGAGCATCC |
| | | | | ATGGAAATTCATCAAGCTCTTACTCGTTCTAATATCATTAGAAATGTT |
| | | | | CTTCCTCGACATGAACAAGGTGGGGTTTTCGCTGCTGAAGGCTACG |
| | | | | CTCGTGCTACTGGACGTGTTGGAGTTTGTATTGCCACTTCTGGTCCG |
| | | | | GGTGCTACTAATCTTGTTTCCGGTTTTGCTGATGCACTTCTTGACTCA |
| | | | | GTCCCGCTTGTCGCCATTACTGGGCAAGTTCCTCGGCGTATGATTGG |
| | | | | TACTGATGCTTTTCAAGAGACTCCTATTGTTGAGGTAACTCGATCAA |
| | | | | TTACTAAGCATAATTATTTGGTGTTAGATGTTGAGGATATCCCTAGA |
| | | | | ATTGTTAAGGAAGCTTTCTTTTTAGCTAATTCTGGTAGACCTGGACC |
| | | | | TGTTTTGATTGATATTCCTAAAGATATTCAGCAACAATTGGTTGTTCC |
| | | | | TAACTGGGAACAGCCCATTAAATTGGGTGGGTATCTTTCTAGGTTG |
| | | | | CCTAAACCCACTTTTTCTGCTAATGAAGAGGGACTTCTTGATCAAAT |
| | | | | TGTGAGGTTGGTGGGTGAGTCTAAGAGACCTGTGCTGTATACTGGA |
| | | | | GGTGGGTGTTTGAATTCTAGTGAAGAATTAAGGAAATTTGTCGAGT |
| | | | | TGACAGGGATTCCGGTTGCTAGTACTTTAATGGGGTTGGGGGCTTT |
| | | | | CCCTTGTACTGATGATTTATCACTTCAAATGTTGGGAATGCACGGGA |
| | | | | CTGTGTACGCGAATTACGCGGTGGATAAGGCTGATTTGTTGCTTGC |
| | | | | TTTCGGGGTTAGGTTTGATGATCGAGTGACTGGGAAGCTCGAGGC |
| | | | | GTTTGCTAGCCGGGCTAAGATTGTGCACATCGATATCGATTCTGCTG |
| | | | | AAATCGGGAAGAATAAGCAACCTCATGTGTCGATTTGTGGTGATAT |
| | | | | TAAAGTGGCATTACGGGGTTGAATAATATTTTGGAATCTAGAAAAG |
| | | | | GAAAGGTGAAATTGGATTTCTCTAATTGGAGGGAGGAATTGAATG |
| | | | | AGCAGAAAAAGAAGTTTCCTTTGAGTTTTAAGACTTTCGGGGATGC |
| | | | | AATTCCTCCGCAATATGCCATTCAGGTTCTGGACGAGTTGACGAAG |
| | | | | GGTGATGCGATTGTAAGTACCGGTGTTGGGCAGCACCAAATGTGG |
| | | | | GCTGCCCAATTCTATAAGTACCGAAATCCTCGCCAATGGCTGACCTC |
| | | | | GGGTGGTTTGGGGGCTATGGGGTTTGGTCTACCAGCCGCTATTGGA |
| | | | | GCTGCTGTTGCTCGACCAGATGCGGTGGTTGTAGACATTGATGGGG |
| | | | | ACGGGAGTTTTATCATGAATGTTCAAGAGTTGGCTACGATTAGGGT |
| | | | | GGAGAATCTCCCGGTTAAAATCATGCTCTTGAACAATCAACATTTAG |
| | | | | GTATGGTTGTTCAATTGGAAGATCGATTTTACAAAGCTAACCGGGC |
| | | | | ACATACATACCTCGGAAATCCATCCAATTCTTCCGAAATCTTCCCGG |
| | | | | ATATGCTCAAATTTGCTGAAGCATGTGATATACCAGCAGCCCGTGTT |
| | | | | ACCAAGGTGAGCGATTTAAGGGCTGCAATTCAAACAATGTTGGATA |
| | | | | CTCCAGGACCATATCTGCTGGATGTAATCGTACCACATCAGGAGCA |
| | | | | TGTGCTGCCTATGATCCCTAGCGGTGCCGCCTTCAAGGACACCATCA |
| | | | | CAGAGGGTGATGGAAGAAGGGCTTATTAGTTGGTTGGAGATCTTTA |
| | | | | TTTGGAGAAGCTTTTTTGTATGTATGTTAGTAGTTCCATAAACTTCTA |
| | | | | TATTCTCTGGCCGTTCTCTCGTTTAGCTGTTTTTATGTTAGTTTGTTGT |
| | | | | TTTCATGTTGCTTGTTACTTTGAAAAACCCTTTTGTGTTTAAGACCCA |
| | | | | TTAGCATGAATAATCTTCCTATACTATTGTATGGTTCGATACACGCTA |
| | | | | GTTGTTTCTTTTTATTAACGAGTTAAAGACAACTATTAGTCGGTGTA |
| | | | | AAAAAAAAGAAAAAAAAACAAAACATGTCGGCCGCCTCGGTCTCTA |
| | | | | CTGAGACACGCAACAGGGGATAGG |
| 10 | Amaranthus | cDNA | 2404 | CTTTCTCCATTTTCGCTTAGCTCTCCTCTCACACAAATTACCTCCATTT |
| | rudis | Contig | | CCAACCTTCCAAGCTTTCAACAATGGCGTCCACTTCTTCAAACCCACC |
| | | | | ATTTTCCTATTTTACTAAACCTTACAAAATCCCTAATCGTCAATCCTA |
| | | | | CATTTACGCTCTCCCTTTTTCCAATTCTCATAAACCCACTTCTTCTTCA |
| | | | | ATCCTCCGCCGCGCTCTTCAAATCTCGTCATCTTCTTCTCAATCACCT |
| | | | | AAACCTAAACCTCCTTCCGCTACTATAACTCAATCACCTTCATCTCTC |
| | | | | ACCGATGATAAACCCTCTTCTTTTGTTTCCCGATTTAGCCCTGAGGA |
| | | | | ACCCAGAAAAGGTTGCGATGTTCTCGTTGAAGCTCTTGAACGTGAA |
| | | | | GGTGTTACCGATGTTTTTGCTTACCCTGGTGGAGCTTCCATGGAAAT |
| | | | | CCATCAAGCTCTTACTCGTTCTAATATCATTAGAAATGTTCTTCCTCG |
| | | | | ACATGAACAAGGTGGGGTTTTCGCTGCTGAAGGCTACGCTCGTGCT |
| | | | | ACTGGACGTGTTGGAGTTTGTATTGCCACTTCTGGTCCGGGTGCTAC |
| | | | | TAATCTTGTTTCCGGTTTTGCTGATGCACTTCTTGACTCAGTCCCGCT |
| | | | | TGTCGCCATTACTGGGCAAGTTCCTCGGCGTATGATTGGTACTGAT |
| | | | | GCTTTTCAAGAGACTCCTATTGTTGAGGTAACTCGATCAATTACTAA |
| | | | | GCATAATTATTTGGTGTTAGATGTTGAGGATATCCCTAGAATTGTTA |
| | | | | AGGAAGCTTTCTTTTTAGCTAATTCTGGTAGACCTGGACCTGTTTTG |
| | | | | ATTGATATTCCTAAAGATATTCAGCAACAATTGGTTGTTCCTAACTG |
| | | | | GGAACAGCCCATTAAATTGGGTGGGTATCTTTCTAGGTTGCCTAAA |
| | | | | CCCACTTTTTCTGCTAATGAAGAGGGACTTCTTGATCAAATTGTGAG |
| | | | | GTTGGTGGGTGAGTCTAAGAGACCTGTGCTGTATACTGGAGGTGG |
| | | | | GTGTTTGAATTCTAGTGAAGAATTAAGGAAATTTGTCGAGTTGACA |
| | | | | GGGATTCCGGTTGCTAGTACTTTAATGGGGTTGGGGGCTTTCCCTT |
| | | | | GTACTGATGATTTATCACTTCAAATGTTGGGAATGCACGGGACTGT |
| | | | | GTACGCGAATTACGCGGTGGATAAGGCTGATTTGTTGCTTGCTTTC |
| | | | | GGGGTTAGGTTTGATGATCGAGTGACTGGGAAGCTCGAGGCGTTT |
| | | | | GCTAGCCGGGCTAAGATTGTGCACATCGATATCGATTCTGCTGAAA |
| | | | | TCGGGAAGAATAAGCAACCTCATGTGTCGATTTGTGGTGATATTAA |
| | | | | AGTGGCATTACGGGGTTGAATAATATTTTGGAATCTAGAAAAGGAA |
| | | | | AGGTGAAATTGGATTTCTCTAATTGGAGGGAGGAATTGAATGAGCA |
| | | | | GAAAAAGAAGTTTCCTTTGAGTTTTAAGACTTTCGGGGATGCAATTC |
| | | | | CTCCGCAATATGCCATTCAGGTTCTGGACGAGTTGACGAAGGGTGA |
| | | | | TGCGATTGTAAGTACCGGTGTTGGGCAGCACCAAATGTGGGCTGCC |
| | | | | CAATTCTATAAGTACCGAAATCCTCGCCAATGGCTGACCTCGGGTG |
| | | | | GTTTGGGGGCTATGGGGTTTGGTCTACCAGCCGCTATTGGAGCTGC |
| | | | | TGTTGCTCGACCAGATGCGGTGGTTGTAGACATTGATGGGGACGG |
| | | | | GAGTTTTATCATGAATGTTCAAGAGTTGGCTACGATTAGGGTGGAG |
| | | | | AATCTCCCGGTTAAAATCATGCTCTTGAACAATCAACATTTAGGTAT |
| | | | | GGTTGTTCAATTGGAAGATCGATTTTACAAAGCTAACCGGGCACAT |
| | | | | ACATACCTCGGAAATCCATCCAATTCTTCCGAAATCTTCCCGGATAT |
| | | | | GCTCAAATTTGCTGAAGCATGTGATATACCAGCAGCCCGTGTTACCA |
| | | | | AGGTGAGCGATTTAAGGGCTGCAATTCAAACAATGTTGGATACTCC |
| | | | | AGGACCATATCTGCTGGATGTAATCGTACCACATCAGGAGCATGTG |
| | | | | CTGCCTATGATCCCTAGCGGTGCCGCCTTCAAGGACACCATCACAG |
| | | | | AGGGTGATGGAAGAAGGGCTTATTAGTTGGTTGGAGATCTTTATTT |
| | | | | GGAGAAGCTTTTTTGTATGTATGTTAGTAGTTCCATAAACTTCTATAT |
| | | | | TCTCTGGCCGTTCTCTCGTTTAGCTGTTTTTATGTTAGTTTGTTGTTTT |
| | | | | CATGTTGCTTGTTACTTTGAAAAACCCTTTTGTGTTTAAGACCCATTA |
| | | | | GCATGAATAATCTTCCTATACTATTGTATGGTTCGATACACGCTAGT |
| | | | | TGTTTCTTTTTATTAACGAGTTAAAGACAACTATTAGTCGGTGTAAA |
| | | | | AAAAAAGAAAAAAAAACAAAACATGTCGGCCGCCTCGGTCTCTACT |
| | | | | GAGACACGCAACAGGGGATAGG |
| 11 | Amaranthus | cDNA | 1364 | TCTTCCTTCTTGGTGATTCCCTTTCTCCATTTTCGCTTAGCTCTCCTCT |
| | rudis | Contig | | CACACAAATTACCTCCATTTCCAACCTTCCAAGCTTTCAACAATGGC |
| | | | | GTCCACTTCTTCAAACCCACCATTTTCTATTTTACTAAACCTTACAAA |
| | | | | ATCCCTAATCGTCAATCCTACATTTACGCTCTCCCTTTTTCCAATTCTC |
| | | | | ATAAACCCACTTCTTCTTCAATCCTCCGCCGCGCTCTTCAAATCTCAT |
| | | | | CATCTTCTTCTCAATCACCTAAACCTAAACCTCCTTCCGCTACTATAA |
| | | | | CTCAATCACCTTCATCTCTCACCGATGATAAACCCTCTTCTTTTGTTTC |
| | | | | CAGATTTAGCCCTGAGAACCCAGAAAAGGTTGCGATGTTCTCGTTG |
| | | | | AAGCTCTTGAACGTGAAGGTGTTACCGATGTTTTTGCTTACCCTGGT |
| | | | | GGAGCTTCCATGGAAATCCATCAAGCTCTTACTCGTTCTAATATCAT |
| | | | | TAGAAATGTTCTTCCTCGACATGAACAAGGTGGGGTTTTCGCTGCTG |
| | | | | AAGGCTACGCTCGTGCTACTGGACGTGTTGGAGTTTGTATTGCCAC |
| | | | | TTCTGGTCCGGGTGCTACTAATCTTGTTTCCGGTTTTGCTGATGCACT |
| | | | | TCTTGACTCAGTCCCGCTTGTCGCCATTACTGGGCAAGTTCCTCGGC |
| | | | | GTATGATTGGTACTGATGCTTTTCAAGAGACTCCTATTGTTGAGGTA |
| | | | | ACTCGATCAATTACTAAGCATAATTATTTGGTGTTAGATGTTGAGGA |
| | | | | TATCCCTAGAATTGTTAAGGAAGCTTTCTTTTTAGCTAATTCTGGTAG |
| | | | | ACCTGGACCTGTTTTGATTGATATTCCTAAAGATATTCAGCAACAAT |
| | | | | TGGTTGTTCCTAACTGGGAACAGCCCATTAAATTGGGTGGGTATCTT |
| | | | | TCTAGGTTGCCTAAACCCACTTTTTCTGCTAATGAAGAGGGACTTCT |
| | | | | TGATCAAATTGTGAGGTTGGTGGGTGAGTCTAAGAGACCTGTGCTG |
| | | | | TATACTGGAGGTGGGTGTTTGAATTCTAGTGAAGAATTAAGGAAAT |
| | | | | TTGTCGAGTTGACAGGGATTCCGGTTGCTAGTACTTTAATGGGGTT |
| | | | | GGGGGCTTTCCCTTGTACTGATGATTTATCACTTCAAATGTTGGGAA |
| | | | | TGCACGGGACTGTGTACGCGAATTACGCGGTGGATAAGGCTGATTT |
| | | | | GTTGCTTGCTTTCGGGGTTAGGTTTGATGATCGAGTGACTGGGAAG |
| | | | | CTCGAGGCGTTTGCTAGCCGGGCTAAGATTGTGCACATCGATATCG |
| | | | | ATTCTGCTGAAATCGGGAAGAATAAGCAACCTCATGTGTCGATTTG |
| | | | | TGGTGATATTAAAGTGGCATTACGGGGGTTGAATAATATTTTGGAA |
| 12 | Amaranthus | gDNA | 2528 | TATAAGTACCGAAATCCTCGCCAATGGCTGACCTCGGGTGGTTTGG |
| | rudis | Contig | | GGGCTATGGGGTTTGGTCTACCAGCCGCTATTGGAGCTGCTGTTGC |
| | | | | TCGACCAGATGCGGTGGTTGTAGACATTGATGGGGACGGGAGTTTT |
| | | | | ATCATGAATGTTCAAGAGTTGGCTACGATTAGGGTGGAGAATCTCC |
| | | | | CGGTTAAAATCATGCTCTTGAACAATCAACATTTAGGTATGGTTGTT |
| | | | | CAATGGGAAGATCGATTTTACAAAGCTAACCGGGCACATACATACC |
| | | | | TCGGGAATCCTTCCAAATCTTCAGAAATCTTCCCGGATATGCTGAAA |
| | | | | TTTGCTGAAGCATGTGATATACCAGCAGCCCGTGTTACCAAGGTGA |
| | | | | GCGATTTAAGGGCTGCAATTCAAACAATGTTGGATACTCCAGGACC |
| | | | | ATATCTGCTGGATGTAATCGTACCACATCAGGAGCATGTGCTGCCTA |
| | | | | TGATCCCTAGCGGTGCCGCCTTCAAGGACACCATCACAGAGGGTGA |
| | | | | TGGAAGAAGGGCTTATTAGTTGGTTGGAGATCTTTATAGAGGAGAA |
| | | | | GCTTTTTTGTATGTATGTTAGTAGTTCCATAAACTTCTATATTCTCTG |
| | | | | GCCGTTCTCTCGTTTAGCTGTTTTTATGTTAGTTTGTTGTTTTCATGTT |
| | | | | GCTTGCTACTTTGAAAAACCCTTTTGTGTTTTAGACCCATTAGTATGA |
| | | | | ATAATCTTCCTATATTATTGTATGGTTCGATACACGCTAGTTGTTTCT |
| | | | | TTTTATTAACGAGTTAAGGACAACAATTAGTCGGTGTAACCAAGTTT |
| | | | | GTCTCACATTGCTCGACGTTTTGTTCTCCTTTGATGAATTTGAGGAG |
| | | | | TATTAGGGAGTTTGTTTGATACTAGTGAATCTTCATGTCCTATACTG |
| | | | | TTTTCTCGTCTTAGCGGACATTGGTATTGTATCTCTTGGTGGGTTTTT |
| | | | | TGTGGGTATAGGAGTATTCGATTCGTTGTTATGTTCGACTCCTTCAA |
| | | | | TGCTTTTGGTGGGTTTTTCAAAGAAGCAAAAGGAAGCAGAAATGGC |
| | | | | CTTAGAATGTAAACTGGTGGAATGGTTAGGTACCCTTCTTTTTTTGT |
| | | | | GGGTGGGTGTTGCCTTTTATCAGCCATGTTTTTATGTTCATATTTTGT |
| | | | | GGTCTTTGAGGCCTTATGTTTAGTGTTGGTGTCTTTTTCGTCGTATTC |
| | | | | TGTGTTCCGTTCTGAAATGTGGATCTCAAAGACATAGATGATGAGG |
| | | | | AAGAGGGTCGGGATAAGTGCAGTGTTTTACTCCAAAAGCGAGCCTA |
| | | | | GTAAGGACGGAAACGAGTCAACATTGCGAGATGATACGCGTTCGA |
| | | | | GTAAGACATGAAAATCTGTCTGCCTGCTCAGTGCTCACAATGGTAA |
| | | | | ATTGTACTGTAAATGATTGTTCTGTCATGTGGGTTTTTTCATCTGGA |
| | | | | AATGTGTATCTAAAATGGTTCTTGTCAGTTGATTGTATCATCAATTCA |
| | | | | CCGAGGATTACACCATTTGCTGCAATTTTGCTCATTCCATTTCTAAAT |
| | | | | TTGGCCTCGGGTATTTCGTTTATTAAGGCTCTATTTGATTCTAAAGCC |
| | | | | GATTCGCCTAGTAAATTTTGGGGGTTCGCTCAAAATTGTTGAAAAA |
| | | | | AGCCCAAACTCAATCATAACTCGCTTTTTTCGATTAGCGATCAGCGG |
| | | | | GGAGATTAGCAAATTATGACATTGTCTATGACCATCTTCATAATCAC |
| | | | | TTTCGGCCTTCATTAGTTGGTTCCCATCTGTCATTGTCTCTACACTAT |
| | | | | CCTTATATGATCTTCATCACCAATGCTACTACTAACTAAACCTAGTAG |
| | | | | CTTTCGCTAACTTGCCGTTCTTCAACAACAAACTTCCATCTTAATAAC |
| | | | | TCTCCGTTTCTCTTGAGATCTTCAACAACGAACTCTTTTAACCTCGTA |
| | | | | ACATTACAATGAGTTCCTTTGTTAAAATGTTTCAATAAATTCCAATGA |
| | | | | CTTCAATAAGCCCTAAAGACGTATAATCACTTCTAATATCTTTTAATT |
| | | | | TTTTAACGTGGTCTGATTTAATATCTTTTAATATACTATTTTTTTTAAT |
| | | | | CCCACTCTGATATCAATTGTTGTGAAATTAGGCTCATTGTATGCTAC |
| | | | | AAATTTGAGGGGAAAACTATGAACAATAATTCTTTAACAAACAAGA |
| | | | | AAAATAGAAAGTTAAAAGATAATTACTCATCAAGTAAATCATGTGT |
| | | | | ACTTTTTATTCGTTTCTTGTTGCATATGATGTTTGATTCCAAGAAACA |
| | | | | ATCGAAAACAATCATTTTATTAGTTGTATCATTGATAAGAGTAAGAT |
| | | | | TGCGTACCCTAAATGAGAGTCGCTTATTAAGATTGGGGTAATGCAA |
| | | | | ATGCGGGTCCCTTTGGGTTAGGAAATTAGATGAAAGTTCCTTAGCG |
| | | | | TAAATCTTTATGGTTGGATTGATAGTAGGGCCATAATTAAACTTTGT |
| | | | | TGAATGTTTGACTCAATTTCTAGGAATACTTGAACACAAATTCTTGC |
| | | | | TATAAACGGTCTATAGCATAGAGGGTGTAATGGGCCAACCCATTTT |
| | | | | TACACTTTCCAAAAACAGCAACTGAAATTTCCATTGGG |
| 13 | Amaranthus | gDNA | 1737 | AAAAAAAAAGCAAAGAATTTAGAATCCAAATCAATCTTGATATTCA |
| | rudis | Contig | | AGGGTATTTAAGTAATTAGAGAACAACCATAGTTAAGTTAAGCGCC |
| | | | | TCCACTCATTTCTCTTCGTCCTTCTCGGTGATTCCCTTTCTCCATTTTC |
| | | | | GCTTAGCTCTCCTCTCACACAAATTACCTCCATTTCCAACCTTCCAAG |
| | | | | CTTTCAACAATGGCGTCCACTTCTTCAAACCCACCATTTTCCTATTTT |
| | | | | ACTAAACCTTACAAAATCCCTAATCGTCAATCCTACATTTACGCTCTC |
| | | | | CCTTTTTCCAATTCTCATAAACCCACTTCTTCTTCAATCCTCCGCCGCG |
| | | | | CTCTTCAAATCTCGTCATCTTCTTCTCAATCACCTAAACCTAAACCTCC |
| | | | | TTCCGCTACTATAACTCAATCACCTTCATCTCTCACCGATGATAAACC |
| | | | | CTCTTCTTTTGTTTCCCGATTTAGCCCTGAGGAACCCAGAAAAGGTT |
| | | | | GCGATGTTCTCGTTGAAGCTCTTGAACGTGAAGGTGTTACCGATGT |
| | | | | TTTTGCTTACCCTGGTGGAGCTTCCATGGAAATCCATCAAGCTCTTA |
| | | | | CTCGTTCTAATATCATTAGAAATGTTCTTCCTCGACATGAACAAGGT |
| | | | | GGGGTTTTCGCTGCTGAAGGCTACGCTCGTGCTACTGGACGTGTTG |
| | | | | GAGTTTGTATTGCCACTTCTGGTCCGGGTGCTACTAATCTTGTTTCC |
| | | | | GGTTTTGCTGATGCACTTCTTGACTCAGTCCCGCTTGTCGCCATTACT |
| | | | | GGGCAAGTTCCTCGGCGTATGATTGGTACTGATGCTTTTCAAGAGA |
| | | | | CTCCTATTGTTGAGGTAACTCGATCAATTACTAAGCATAATTATTTG |
| | | | | GTGTTAGATGTTGAGGATATCCCTAGAATTGTTAAGGAAGCTTTCTT |
| | | | | TTTAGCTAATTCTGGTAGAACTGACCTGTTTTGATTGATATTCCTAAA |
| | | | | GATATTCAGCAACAATTGGTTGTTCCTAACTGGGAACAACCCATTAA |
| | | | | ATTGGGTGGGTATCTTTCTAGGTTGCCTAAACCCACTTATTCTCCTTA |
| | | | | TGAAGAGGGACTTCTTGATCAAATTGTGAGGTTGGTGGTTGAAGAA |
| | | | | GCCATTGATGAAGATGGTTGAAGAATTCAAATCAATGTTGGGGTTT |
| | | | | TGAAGGTTTTGTTGTGTGCCATTGATGAAGAGATTTAAGGAAGAAA |
| | | | | TCATTAATGGGTAGATTGAGGAAGAAGGTGATGAAGAAGCTATTG |
| | | | | ATGAAGATGGTAACATGTTGTTCCAGGTAACTAAAACCGATTGTTCT |
| | | | | TTAAAAGCGGAGACCCCCAAAGTTCGAGTCTTTAAAAGATAAACGC |
| | | | | AAGTTTAAACCTTTAAAAGCAAATGAGCTAAAGTTCGTACCTTTAGA |
| | | | | ATCAAATTTTCCAAAATAATATTAACCATCTTATCGATTTGCGTGCTG |
| | | | | AAAAATCGGCGCCTAAATATTCATTTCGATTGTATTTTCAAACTAGG |
| | | | | AGCCCTATAAGAATTATAATTATGTCCATATTTCAAAAAAAAATTAC |
| | | | | ACTATTAAAAAATTTAGGAAAAATTACCTAAAATAATCCAATCTTTT |
| | | | | ATTCATTTTTCTAGAATAATCCTAACTTTTGATTAACCATGAATAATA |
| | | | | AAACCAATTTTAGGGTCACTTATTCAAGAACATTGTTACCCAAATAG |
| | | | | TGACTGATTTTTGTAGGTTTTTTTTTCGGAAATTCGGAATTAGTATAT |
| | | | | ATGAAAAAGAAAAAAAGAAAACACTGAATCATGCATCCTT |
| 14 | Amaranthus | gDNA | 1719 | AAAAAAAAAAAAAAAAGCAAAGAATTTAGAATCCAAATCAATCTTG |
| | rudis | Contig | | ATATTCAAGGGTATTTAAGTAATTAGAGAACAACCATAGTTAAGTTA |
| | | | | AGCGCCTCCACTCATTTCTCTTCGTCCTTCTCAGTGATTCCCTTTCTCC |
| | | | | ATTTTCGCTTAGCTCTCCTCTCACACAAATTACCTCCATTTCCAACCTT |
| | | | | CCAAGCTTTCAACAATGGCGTCCACTTCTTCAAACCCACCATTTTTCT |
| | | | | ATTTTACTAAACCTTACAAAATCCCTAATCGTCAATCCTACATTTACG |
| | | | | CTCTCCCTTTTTCCAATTCTCATAAACCCACTTCTTCTTCTTCTTCTTCT |
| | | | | TCAATCGTCCGCCGCGCTCTTCAAATCTCATTATCTTCTTCTCAATCA |
| | | | | CCTAAACCTAAACCTCCTTCCGCTACTATAACTCAATCACCTTCATCT |
| | | | | CTCACCGATGATAAACCCTCTTCTTTTGTTTCCCGATTTAGCCCTGAT |
| | | | | GAACCCAGAAAAGGTTGCGATGTTCTTGTTGAAGCTCTTGAACGTG |
| | | | | AAGGTGTTACCGATGTTTTTGCTTACCCTGGTGGAGCTTCCATGGAA |
| | | | | ATCCATCAAGCTCTTACTCGTTCTAATATCATTAGAAATGTTCTTCCT |
| | | | | CGACATGAACAAGGTGGGGTTTTCGCTGCTGAAGGCTACGCTCGTG |
| | | | | CTACTGGACGTGTTGGAGTTTGTATTGCCACTTCTGGTCCGGGTGCT |
| | | | | ACTAATCTTGTTTCCGGTTTTGCTGATGCACTTCTTGACTCAGTCCCG |
| | | | | CTTGTCGCCATTACTGGGCAAGTTCCTCGGCGTATGATTGGTACTGA |
| | | | | TGCTTTTCAAGAGACTCCTATTGTTGAGGTAACTCGATCAATTACTA |
| | | | | AGCATAATTATTTGGTGTTAGATGTTGAGGATATCCCTAGAATTGTT |
| | | | | AAGGAAGCTTTCTTTTTAGCTAATTCTGGTAGACCTGGACCTGTTTT |
| | | | | GATTGATATTCCTAAAGATATTCAGCAACAATTGGTTGTTCCTAACT |
| | | | | GGGAACAGCCCATTAAATTGGGTGGGTATCTTTCTAGGTTGCCTAA |
| | | | | ACCCACTTATTCTGCTAATGAAGAGGGACTTCTTGATCAAATTGTGA |
| | | | | GGTTGGTGGGTGAGTCTAAGAGACCTGTGCTGTATACTGGAGGTG |
| | | | | GGTGTTTGAATTCTAGTGAAGAATTAAGGAAATTTGTCGGGTTGAC |
| | | | | AGGGATTCCGGTTGCTAGTACTTTAATGGGGTTGGGGGCTTTCCCT |
| | | | | TGTACTGATGATTTATCACTTCAAATGTTGGGAATGCACGGGACTGT |
| | | | | GTACGCGAATTACGCGGTGGATAAGGCTGATTTGTTGCTTGCTTTC |
| | | | | GGGGTTAGGTTTGATGATCGAGTGACTGGGAAGCTCGAGGCGTTT |
| | | | | GCTAGCCGGGCTAAGATTGTGCACATCGATATTGATTCTGCTGAAA |
| | | | | TCGGGAAGAATAAGCAACCTCATGTGTCGATTTGTGGTGATATTAA |
| | | | | AGTGGCATTACAGGGGTTGAATAAGATTTTGGAATCTAGAAAAGG |
| | | | | AAAGCTGAAATTGGATTTCTCTAATTGGAGGGAGGAGTTGAATGAG |
| | | | | CAGAAAAAGAAGTTTCCTTTGAGTTTTAAGACTTTCGGGGATGCAA |
| | | | | TTCCTCCGCAATATGCCATTCAGGTTCTGGACGAGTTGACGAAGGG |
| | | | | TGATGCGATTGTAAGTACCGGTGTTGGGCAGCACCAAATGTGGGCT |
| | | | | GCCCAATTCTATAAGTACCGAAATCCTCGCCA |
| 15 | Amaranthus | gDNA | 725 | AAATTTGCTGAAGCATGTGATATACCAGCAGCCCGTGTTACCAAGG |
| | rudis | Contig | | TGAGCGATTTAAGGGCTGCAATTCAAACAATGTTGGATACTCCAGG |
| | | | | ACCATATCTGCTGGATGTAATCGTACCACATCAGGAGCATGTGCTG |
| | | | | CCTATGATCCCTAGCGGTGCCGCCTTCAAGGACACCATCACAGAGG |
| | | | | GTGATGGAAGAAGGGCTTATTAGTTGGTTGGAGATCTTTATAGAGG |
| | | | | AGAAGCTTTTTTGTATGTATGTTAGTAGTTCCATAAACTTCTATATTC |
| | | | | TCTGGCCGTCCTCTCGTTTAGCTGTTTTTATGTTAGTTTATTGTTTTCA |
| | | | | TGTTGCTTGTTACTTTGAAAAACCCTTTTGTGTTTTAAGACCCATTAG |
| | | | | CATGAATAATCTTCCTATACTATTGTATGGTTCGATACACGCTAGTT |
| | | | | GTTTCTTTTTATTAACGAGTTAAAGACAACTATTAGTCGGTGTAATC |
| | | | | AAGTCTGTCTCACATTGCTTGACATTTTGTTCTCCTTTGATGAATTTG |
| | | | | ATATTAGGAAGTTTGTTTGGTACTAGTGAATCTTCATGTCCTATACT |
| | | | | GTTTTCTCGTCTTAGCGGACATTGGTATTGTATCTCTTGGTGGGTTTT |
| | | | | TTGTGGGTATAGGAGTATTCGATTCGTTGTTATGTTCGACTCCTTCA |
| | | | | ATGCTTTTGGTGGGTTTTTCAAAGAAGAAAAAGGAAGCAAAAATGG |
| | | | | CCTTAGAATGTAAAGTGGTG |
| 16 | Amaranthus | gDNA | 451 | TTAAAGTGGCATTACGGGGGTTGAATAATATTTTGGAATCTAGAAA |
| | rudis | Contig | | AGGAAAGGTGAAATTGGATTTCTCTAATTGGAGGGAGGAGTTGAA |
| | | | | TGAGCAGAAAAAGAAGTTTCCTTTGAGTTTTAAGACTTTCGGGGAT |
| | | | | GCAATTCCTCCGCAATATGCCATTCAGGTTCTGGACGAGTTGACGA |
| | | | | AGGGTGATGCGATTGTAAGTACCGGTGTTGGGCAGCACCAAATGT |
| | | | | GGGCTGCCCAATTCTATAAGTACCGAAATCCTCGCCAATGGCTGAC |
| | | | | CTCGGGTGGTTTGGGGGCTATGGGGTTTGGTCTACCAGCCGCTATT |
| | | | | GGAGCTGCTGTTGCTCGACCAGATGCGGTGGTTGTAGACATTGATG |
| | | | | GGGACGGGAGTTTTATCATGAATGTTCAAGAGTTGGCTACGATTAG |
| | | | | GGTAGAGAATCTCCCGGTTAAAATCATGCTCTTGAACAA |
| 17 | Amaranthus | gDNA | 375 | TCTTTCTAGGTTGCCTAAACCCACTTTTTCTGCTAATGAAGAGGGAC |
| | rudis | Contig | | TTCTTGATCAAATTGTGAGGTTGGTGGGTGAGTCTAAGAGACCTGT |
| | | | | GCTGTATACTGGAGGTGGGTGTTTGAATTCTAGTGAAGAATTAAGG |
| | | | | AAATTTGTCGAGTTGACAGGGATTCCGGTTGCTAGTACTTTAATGG |
| | | | | GGTTGGGGGCTTTCCCTTGTACTGATGATTTATCACTTCAAATGTTG |
| | | | | GGAATGCACGGGACTGTGTACGCGAATTACGCGGTGGATAAGGCT |
| | | | | GATTTGTTGCTTGCTTTCGGGGTTAGGTTTGATGATCGAGTGACTG |
| | | | | GGAAGCTCGAGGCGTTTGCTAGCCGGGCTAAGATTGTGCACATCGA |
| | | | | TATCGA |
| 18 | Amaranthus | cDNA | 2302 | CTTCCTTCTCAGTTATTCCATTTCTCCATTCTCGCTTAGCTTTCCTCTCT |
| | spinosus | Contig | | CACAAATTACCTCCATTTCCAAGCTTCCAAGCTTTCAACAATGGCGT |
| | | | | CCAGTTCTTCAAACCCACCATTATTCTATTTTACTAAACTTAACAAAA |
| | | | | TCCCTAATCGGCAATCATCCATTTACGCTATCCCGTTTTCTAATTCTCT |
| | | | | TAAACCCACTTCTTCTTCTTCAATCCTCCGCCGCCCTCTTCAAATCTCA |
| | | | | TCATCTTCTTCTCAATCACCTAAACCTAAACCTCCTTCCGCTACTATA |
| | | | | ACTCAGTCACCTTCATCTCTCACCGATGATAAACCCTCTTCTTTTGTTT |
| | | | | CCCGATTTAGCCCTGAAGAACCCAGAAAAGGTTGCGATGTTCTCGT |
| | | | | TGAAGCTCTTGAACGTGAAGGTGTTACCGATGTTTTTGCTTACCCTG |
| | | | | GTGGAGCATCCATGGAAATCCATCAAGCTCTTACTCGTTCTAATATC |
| | | | | ATTAGAAATGTTCTTCCTCGACATGAACAAGGTGGGGTTTTCGCTGC |
| | | | | TGAAGGCTACGCTCGTGCTACTGGACGCGTTGGAGTTTGTATTGCC |
| | | | | ACTTCTGGTCCAGGTGCTACTAATCTTGTCTCTGGTCTTGCTGATGC |
| | | | | ACTTCTTGACTCAGTCCCGCTTGTCGCCATTACTGGGCAAGTTCCCC |
| | | | | GGCGTATGATTGGTACTGATGCTTTTCAAGAGACTCCAATTGTTGAG |
| | | | | GTAACTCGATCCATTACTAAGCATAATTATTTGGTGTTAGATGTTGA |
| | | | | GGATATTCCTAGAATTGTTAAGGAAGCTTTCTTTTTAGCTAATTCTG |
| | | | | GTAGACCTGGACCTGTTTTGATTGATATTCCTAAAGATATTCAGCAA |
| | | | | CAATTAGTTGTTCCTAATTGGGAACAGCCCATTAAATTGGGTGGGT |
| | | | | ATCTTTCTAGGTTGCCTAAACCCACTTATTCTGCTAATGAAGAGGGA |
| | | | | CTTCTTGATCAAATTGTAAGGTTAGTGGGTGAGTCTAAGAGACCTG |
| | | | | TGCTGTATACTGGAGGTGGGTGTTTGAATTCTAGTGAAGAATTGAG |
| | | | | GAAATTTGTCAAATTGACAGGGATTCCGGTGGCTAGTACTTTAATG |
| | | | | GGGTTGGGGGCTTTCCCTTGTACTGATGATTTATCACTTCATATGCT |
| | | | | GGGAATGCACGGGACTGTGTATGCGAATTACGCGGTTGATAAGGC |
| | | | | CGATTTGTTGCTTGCTTTCGGGGTTAGGTTTGATGATCGAGTGACTG |
| | | | | GTAAGCTCGAGGCGTTTGCTAGCCGGGCTAAGATTGTGCACATCGA |
| | | | | TATCGATTCTGCTGAAATCGGGAAGAATAAGCAACCTCATGTGTCG |
| | | | | ATTTGTGGTGATGTTAAAGTGGCATTACAGGGGTTGAATAAGATTT |
| | | | | TGGAATCTAGAAAAGGAAAGGTGAAATTGGATTTCTCTAATTGGAG |
| | | | | GGAGGAGTTGAATGAGCAGAAAGAGAAGTTTCCTTTGAGTTTTAAG |
| | | | | ACTTTCGGGGATGCAATTCCTCCGCAATACGCCATTCAGGTTCTTGA |
| | | | | CGAGTTGACGAAGGGTGATGCGGTTGTAAGTACCGGTGTTGGGCA |
| | | | | GCACCAAATGTGGGCTGCCCAATTCTATAAGTACCGAAATCCTCGCC |
| | | | | AATGGCTGACCTCGGGTGGTTTGGGGGCTATGGGGTTTGGTCTACC |
| | | | | TGCTGCTATTGGAGCTGCTGTTGCTCGACCAGATGCGGTGGTTGTA |
| | | | | GACATCGATGGGGATGGGAGTTTTATCATGAATGTTCAAGAGTTGG |
| | | | | CTACGATTAGGGTGGAGAATCTCCCGGTTAAAATCATGCTCTTGAA |
| | | | | CAATCAACATTTAGGTATGGTTGTTCAATGGGAAGATCGATTTTACA |
| | | | | AAGCTAACCGGGCACATACATACCTCGGGAATCCTTCCAATTCTTCC |
| | | | | GAAATCTTCCCGGATATGCTCAAATTCGCTGAAGCATGTGATATACC |
| | | | | AGCAGCTCGTGTTACCAAGGTGAGCGATTTAAGGGCTGCAATTCAA |
| | | | | ACAATGTTGGATACTCCAGGACCGTATCTGCTGGATGTAATCGTACC |
| | | | | ACATCAGGAGCATGTGCTGCCTATGATCCCTAGCGGTGCCGCCTTC |
| | | | | AAGGACACCATCACTGAGGGTGATGGAAGAAGGGCTTATTAGTTG |
| | | | | GTTGGAGATCTTTATAGAGGAGAAGCTTTTTGTATGTATGTTAGTA |
| | | | | GTTCCATAAACTTCTATATTCTCTGGCCGGTCTCTCGTGTAGCTGTTT |
| | | | | TTATGTTAGTTTGTTGTTTTCATGTTGCGTGCTACTTTGAAAAAACCC |
| | | | | TTTTGTGTTTTAGACCCATTAGCATGAATAATCATCCTATATTATTGT |
| | | | | ATGGTTCGA |
| 19 | Amaranthus | cDNA | 2334 | TCTGGGTGATTCCCTTTCTCCATTCTCGCTTAACTTTCCTCTCACACA |
| | thunbergii | Contig | | AATTACCTTCATTTCCAACCTTTCAAGCTTTCAACAATGGCGTCCAAT |
| | | | | TCTTCAAACCCACCATTTTTCTATTTTACTATACCTTACAAAATCCCTA |
| | | | | ATCTGCAATCATCCATTTACGCTATCCCTTTTTCCGATTCTCTTAAACC |
| | | | | CACTTCTTCTTCTTCAATCCTCCGCCGCCCTCTTCAAATCTCATCATCT |
| | | | | TCTTCTCAATCACCTAAACCTAAACCTCCTTCCGCTACTATAACTCAA |
| | | | | TCACCTTCGTCTCTCACCGATGATAAACCCTCTTCTTTTGTTTCCCGA |
| | | | | TTTAGCCCTGAAGAACCCAGAAAAGGTTGCGATGTTCTCGTTGAAG |
| | | | | CTCTTGAACGTGAAGGTGTTACCGATGTTTTTGCTTACCCTGGTGGA |
| | | | | GCATCCATGGAAATTCATCAAGCTCTTACTCGTTCTAATATCATTAG |
| | | | | AAATGTTCTTCCTCGACATGAACAAGGTGGGGTTTTCGCTGCTGAA |
| | | | | GGCTACGCTCGTGCTACTGGACGCGTTGGAGTTTGTATTGCCACTTC |
| | | | | TGGTCCAGGTGCTACTAATCTTGTTTCTGGTCTTGCTGATGCACTTCT |
| | | | | TGACTCAGTCCCTCTTGTCGCCATTACTGGGCAAGTTCCCCGGCGTA |
| | | | | TGATTGGTACTGATGCTTTTCAAGAGACTCCAATTGTTGAGGTAACT |
| | | | | CGATCCATTACCAAGCATAATTATTTGGTGTTAGATGTTGAGGATAT |
| | | | | TCCTAGAATTGTTAAGGAAGCTTTCTTTTTAGCTAATTCTGGTAGAC |
| | | | | CTGGACCTGTTTTGATTGATATTCCTAAAGATATTCAGCAACAATTA |
| | | | | GTTGTTCCTAATTGGGAACAGCCCATTAAATTGGGTGGGTATCTTTC |
| | | | | TAGGTTGCCTAAACCCACTTATTCTGCTAATGAAGAGGGACTTGATC |
| | | | | AAATTGTAAGGTTAGTGGGTGAGTCTAAGAGACCTGTGCTGTATAC |
| | | | | TGGAGGTGGGTGTTTGAATTCTAGTGAAGAATTGAGGAAATTTGTC |
| | | | | GAATTGACAGGTATTCCGGTGGCTAGTACTTTAATGGGGTTGGGGG |
| | | | | CTTTCCCTTGTACTGATGATTTATGTCTTCATATGTTGGGAATGCACG |
| | | | | GGACTGTGTACGCGAATTACGCGGTTGATAAGGCCGATTTGTTGCT |
| | | | | TGCTTTTGGGGTTAGGTTTGATGATCGAGTGACTGGTAAGCTCGAG |
| | | | | GCGTTTGCTAGCCGGGCTAAGATTGTGCACATCGATATCGATTCTGC |
| | | | | TGAAATCGGGAAGAATAAGCAACCTCATGTGTCGATTTGTGGTGAT |
| | | | | GTTAAAGTGGCATTACAGGGGTTGAATAAGATTTTGGAATCTAGAA |
| | | | | AAGGAAAGGTGAAATTGGATTTCTCTAATTGGAGGGAGGAGTTGA |
| | | | | ATGAGCAGAAAAAGAAGTTTCCTTTGAGTTTTAAGACTTTCGGGGA |
| | | | | TGCAATTCCTCCGCAATACGCCATTCAGGTTCTTGACGAGTTGACGA |
| | | | | AGGGCGATGCGGTTGTAAGTACTGGTGTTGGGCAGCACCAAATGT |
| | | | | GGGCTGCCCAATTCTATAAGTACCGAAATCCTCGCCAATGGCTGAC |
| | | | | CTCGGGTGGTTTGGGGGCTATGGGGTTTGGTCTACCAGCTGCTATT |
| | | | | GGAGCTGCTGTTGCTCGACCAGATGCGGTGGTTGTAGACATTGATG |
| | | | | GGGATGGGAGTTTTATCATGAATGTTCAAGAGTTGGCTACGATTAG |
| | | | | GGTAGAGAATCTCCCGGTTAAAATCATGCTCTTGAACAATCAACATT |
| | | | | TAGGTATGGTTGTTCAATGGGAAGATCGATTTTACAAAGCTAACCG |
| | | | | GGCACATACATACCTCGGGAATCCTTCCAATTCTTCCGAAATCTTCC |
| | | | | CGGATATGCTCAAATTTGCTGAAGCATGTGATATACCAGCAGCCCG |
| | | | | TGTTACCAAGGTGAGCGATTTAAGGGCTGCAATTCAAACAATGTTG |
| | | | | GATACTCCAGGACCGTATCTGCTGGATGTAATCGTACCACATCAGG |
| | | | | AGCATGTGCTGCCTATGATCCCTAGCGGTGCCGCCTTCAAGGACAC |
| | | | | CATAACAGAGGGTGATGGAAGAAGGGCTTATTAGTTGGTTGGAGA |
| | | | | TCTTTATAGAGGAGAATCTTTTTTGTATGTATGTTAGTAGTTCCATAA |
| | | | | ACTTCTATATTCTCTGGCCGTTCTCTCGTTTAGCTGTTTTTATGTTAGT |
| | | | | TTGTTGTTTTCATGTTGCTTGCTACTTTGAAAAACCCTTTTGTGTTTTA |
| | | | | GACCCATTAGCATGAATAATCTTCCTATATTATTGTATGGTTCGATAC |
| | | | | ACGCTAGTTGTTTCTTTTTATTATCGAGTTAAAGACAA |
| 20 | Amaranthus | cDNA | 2360 | CGCCTCCACTCTGGGTGATTCCCTTTCTCCATTCTCGCTTAGCTTTCC |
| | viridis | Contig | | TCTCACACAAATTACCTTTATTTCCAACCTTTCAAGCTTTCAACAATG |
| | | | | GCGTCCACTTCTTCAAACCCACCATTTTCCTCTTTTACTAAACCTAAC |
| | | | | AAAATCCCTAATCTGCAATCATCCATTTACGCTATCCCTTTTTCCAAT |
| | | | | TCTCTTAAACCCACTTCTTCTTCTTCAATCCTCCGCCGCCCTCTTCAAA |
| | | | | TCTCATCATCTTCTTCTCAATCACCTAAACCTAAACCTCCTTCCGCTAC |
| | | | | TATAACTCAATCACCTTCGTCTCTCACCGATGATAAACCCTCTTCTTT |
| | | | | TGTTTCCCGATTTAGCCCTGAAGAACCCAGAAAAGGTAGCGATGTT |
| | | | | CTCGTTGAAGCTCTTGAACGTGAAGGTGTTACCGATGTTTTTGCTTA |
| | | | | CCCTGGTGGAGCATCCATGGAAATTCATCAAGCTCTTACTCGTTCTA |
| | | | | ATATCATTAGAAATGTTCTTCCTCGACATGAACAAGGTGGGGTTTTC |
| | | | | GCTGCTGAAGGCTACGCTCGTGCTACTGGACGCGTTGGAGTTTGTA |
| | | | | TTGCCACTTCTGGTCCAGGTGCTACTAATCTTGTTTCTGGTCTTGCTG |
| | | | | ATGCACTTCTTGACTCAGTCCCTCTTGTCGCCATTACTGGGCAAGTT |
| | | | | CCCCGGCGTATGATTGGTACTGATGCTTTTCAAGAGACTCCAATTGT |
| | | | | TGAGGTAACTCGATCCATTACCAAGCATAATTATTTGGTGTTAGATG |
| | | | | TTGAGGATATTCCTAGAATTGTTAAGGAAGCTTTCTTTTTAGCTAATT |
| | | | | CTGGTAGACCTGGACCTGTTTTGATTGATATTCCTAAAGATATTCAG |
| | | | | CAACAATTAGTTGTTCCTAATTGGGAACAGCCCATTAAATTGGGTG |
| | | | | GGTATCTTTCTAGGTTGCCTAAACCCACTTATTCTGCTAATGAAGAG |
| | | | | GGACTTCTTGATCAAATTGTAAGGTTAGTGGGTGAGTCTAAGAGAC |
| | | | | CTGTGCTGTATACTGGAGGTGGGTGTTTGAATTCTAGTGAAGAATT |
| | | | | GAGGAAATTTGTCGAATTGACAGGTATTCCGGTGGCTAGTACTTTA |
| | | | | ATGGGGTTGGGGGCTTTCCCTTGTACTGATGATTTATGTCTTCATAT |
| | | | | GTTGGGAATGCACGGGACTGTGTACGCGAATTACGCGGTTGATAA |
| | | | | GGCCGATTTGTTGCTTGCTTTTGGGGTTAGGTTTGATGATCGAGTG |
| | | | | ACTGGTAAGCTCGAGGCGTTTGCTAGCCGGGCTAAGATTGTGCACA |
| | | | | TCGATATCGATTCTGCTGAAATCGGGAAGAATAAGCAACCTCATGT |
| | | | | GTCGATTTGTGGTGATGTTAAAGTGGCATTACAGGGGTTGAATAAG |
| | | | | ATTTTGGAATCTAGAAAAGGAAAGGTGAAATTGGATTTCTCTAATT |
| | | | | GGAGGGAGGAGTTGAATGAGCAGAAAAAGAAGTTTCCTTTGAGTT |
| | | | | TTAAGACTTTCGGGGATGCAATTCCTCCGCAATACGCCATTCAGGTT |
| | | | | CTTGACGAGTTGACGAAGGGCGATGCGGTTGTAAGTACTGGTGTTG |
| | | | | GGCAGCACCAAATGTGGGCTGCCCAATTCTATAAGTACCGAAATCC |
| | | | | TCGCCAATGGCTGACCTCGGGTGGTTTGGGGGCTATGGGGTTTGGT |
| | | | | CTACCAGCTGCTATTGGAGCTGCTGTTGCTCGACCAGATGCGGTGG |
| | | | | TTGTAGACATTGATGGGGATGGGAGTTTTATCATGAATGTTCAAGA |
| | | | | GTTGGCTACGATTAGGGTAGAGAATCTCCCGGTTAAAATCATGCTC |
| | | | | TTGAACAATCAACATTTAGGTATGGTTGTTCAATGGGAAGATCGATT |
| | | | | TTACAAAGCTAACCGGGCACATACATACCTCGGGAATCCTTCCAATT |
| | | | | CTTCCGAAATCTTCCCGGATATGCTCAAATTTGCTGAAGCATGTGAT |
| | | | | ATACCAGCAGCCCGTGTTACCAAGGTGAGCGATTTAAGGGCTGCAA |
| | | | | TTCAAACAATGTTGGATACTCCAGGACCGTATCTGCTGGATGTAATC |
| | | | | GTACCACATCAGGAGCATGTGCTGCCTATGATCCCTAGCGGTGCCG |
| | | | | CCTTCAAGGACACCATAACAGAGGGTGATGGAAGAAGGGCTTATT |
| | | | | AGTTGGTTGGAGATCTTTATAGAGGAGAATCTTTTTTGTATGTATGT |
| | | | | TAGTAGTTCCATAAACTTCTATATTCTCTGGCCGTTCTCTCGTTTAGC |
| | | | | TGTTTTTATGTTAGTTTGTTGTTTTCATGTTGCTTGCTACTTTGAAAA |
| | | | | ACCCTTTTGTGTTTTAGACCCATTAGCATGAATAATCTTCCTATATTA |
| | | | | TTGTATGGTTCGATACACGCTAGTTGTTTCTTTTTATTATCGAGTTAA |
| | | | | AGACAACTACTAATCGGTGT |
| 21 | Amaranthus | cDNA | 2357 | CTTCCTTCTCAGTTATTCCATTTCTCCATTCTCGCTTAGCTTTCCTCTCT |
| | viridis | Contig | | CACAAATTACCTCCATTTCCAAGCTTCCAAGCTTTCAACAATGGCGT |
| | | | | CCAGTTCTTCAAACCCACCATTATTCTATTTTACTAAACTTAACAAAA |
| | | | | TCCCTAATCGGCAATCATCCATTTACGCTATCCCGTTTTCTAATTCTCT |
| | | | | TAAACCCACTTCTTCTTCTTCAATCCTCCGCCGCCCTCTTCAAATCTCA |
| | | | | TCATCTTCTTCTCAATCACCTAAACCTAAACCTCCTTCCGCTACTATA |
| | | | | ACTCAGTCACCTTCATCTCTCACCGATGATAAACCCTCTTCTTTTGTTT |
| | | | | CCCGATTTAGCCCTGAAGAACCCAGAAAAGGTTGCGATGTTCTCGT |
| | | | | TGAAGCTCTTGAACGTGAAGGTGTTACCGATGTTTTTGCTTACCCTG |
| | | | | GTGGAGCATCCATGGAAATCCATCAAGCTCTTACTCGTTCTAATATC |
| | | | | ATTAGAAATGTTCTTCCTCGACATGAACAAGGTGGGGTTTTCGCTGC |
| | | | | TGAAGGCTACGCTCGTGCTACTGGACGCGTTGGAGTTTGTATTGCC |
| | | | | ACTTCTGGTCCAGGTGCTACTAATCTTGTTTCTGGTCTTGCTGATGC |
| | | | | ACTTCTTGACTCAGTCCCTCTTGTCGCCATTACTGGGCAAGTTCCCC |
| | | | | GGCGTATGATTGGTACTGATGCTTTTCAAGAGACTCCAATTGTTGAG |
| | | | | GTAACTCGATCCATTACCAAGCATAATTATTTGGTGTTAGATGTTGA |
| | | | | GGATATTCCTAGAATTGTTAAGGAAGCTTTCTTTTTAGCTAATTCTG |
| | | | | GTAGACCTGGACCTGTTTTGATTGATATTCCTAAAGATATTCAGCAA |
| | | | | CAATTAGTTGTTCCTAATTGGGAACAGCCCATTAAATTGGGTGGGT |
| | | | | ATCTTTCTAGGTTGCCTAAACCCACTTATTCTGCTAATGAAGAGGGA |
| | | | | CTTCTTGATCAAATTGTAAGGTTAGTGGGTGAGTCTAAGAGACCTG |
| | | | | TGCTGTATACTGGAGGTGGGTGTTTGAATTCTAGTGAAGAATTGAG |
| | | | | GAAATTTGTCGAATTGACAGGTATTCCGGTGGCTAGTACTTTAATG |
| | | | | GGGTTGGGGGCTTTCCCTTGTACTGATGATTTATGTCTTCATATGTT |
| | | | | GGGAATGCACGGGACTGTGTACGCGAATTACGCGGTTGATAAGGC |
| | | | | CGATTTGTTGCTTGCTTTTGGGGTTAGGTTTGATGATCGAGTGACTG |
| | | | | GTAAGCTCGAGGCGTTTGCTAGCCGGGCTAAGATTGTGCACATCGA |
| | | | | TATCGATTCTGCTGAAATCGGGAAGAATAAGCAACCTCATGTGTCG |
| | | | | ATTTGTGGTGATGTTAAAGTGGCATTACAGGGGTTGAATAAGATTT |
| | | | | TGGAATCTAGAAAAGGAAAGGTGAAATTGGATTTCTCTAATTGGAG |
| | | | | GGAGGAGTTGAATGAGCAGAAAAAGAAGTTTCCTTTGAGTTTTAAG |
| | | | | ACTTTCGGGGATGCAATTCCTCCGCAATACGCCATTCAGGTTCTTGA |
| | | | | CGAGTTGACGAAGGGCGATGCGGTTGTAAGTACTGGTGTTGGGCA |
| | | | | GCACCAAATGTGGGCTGCCCAATTCTATAAGTACCGAAATCCTCGCC |
| | | | | AATGGCTGACCTCGGGTGGTTTGGGGGCTATGGGGTTTGGTCTACC |
| | | | | AGCTGCTATTGGAGCTGCTGTTGCTCGACCAGATGCGGTGGTTGTA |
| | | | | GACATTGATGGGGATGGGAGTTTTATCATGAATGTTCAAGAGTTGG |
| | | | | CTACGATTAGGGTAGAGAATCTCCCGGTTAAAATCATGCTCTTGAAC |
| | | | | AATCAACATTTAGGTATGGTTGTTCAATGGGAAGATCGATTTTACAA |
| | | | | AGCTAACCGGGCACATACATACCTCGGGAATCCTTCCAATTCTTCCG |
| | | | | AAATCTTCCCGGATATGCTCAAATTTGCTGAAGCATGTGATATACCA |
| | | | | GCAGCCCGTGTTACCAAGGTGAGCGATTTAAGGGCTGCAATTCAAA |
| | | | | CAATGTTGGATACTCCAGGACCGTATCTGCTGGATGTAATCGTACCA |
| | | | | CATCAGGAGCATGTGCTGCCTATGATCCCTAGCGGTGCCGCCTTCA |
| | | | | AGGACACCATAACAGAGGGTGATGGAAGAAGGGCTTATTAGTTGG |
| | | | | TTGGAGATCTTTATAGAGGAGAATCTTTTTTGTATGTATGTTAGTAG |
| | | | | TTCCATAAACTTCTATATTCTCTGGCCGTTCTCTCGTTTAGCTGTTTTT |
| | | | | ATGTTAGTTTGTTGTTTTCATGTTGCTTGCTACTTTGAAAAACCCTTT |
| | | | | TGTGTTTTAGACCCATTAGCATGAATAATCTTCCTATATTATTGTATG |
| | | | | GTTCGATACACGCTAGTTGTTTCTTTTTATTATCGAGTTAAAGACAAC |
| | | | | TACTAATCGGTGT |
| 22 | Ambrosia | cDNA | 2075 | CACTCATTCAACAATGGCGGCCATCTCTCCCACAAACCCTTCCTTCAC |
| | trifida | Contig | | CACCAAACCGCCGTCATCTTCCGCCACCACACCACCACCACGTTCCA |
| | | | | CCTTCCTCCCCCGTTTCACATTCCCAATAACCTCCACTTCCCCAATAC |
| | | | | GACACCGTTTCCACATATCCAACGTTCTCTCCGACCACAAACCCACC |
| | | | | ATAACCCATTCCCCATCACCAACCGACCCATTCATCTCCCGTTATGCC |
| | | | | CCAGACCAGCCCCGTAAAGGCGCCGACGTCCTAGTCGAAGCTTTGG |
| | | | | AACGTGAAGGCGTCACCGACGTCTTCGCATACCCAGGAGGCGCCTC |
| | | | | AATGGAGATCCATCAAGCTCTGACCCGCTCCAAAACCATCCGAAAC |
| | | | | GTCCTTCCCCGTCATGAACAGGGCGGCGTCTTCGCCGCCGAAGGCT |
| | | | | ACGCACGCGCCTCCGGTCTTCCTGGCGTCTGTATTGCTACCTCTGGT |
| | | | | CCTGGAGCTACAAACCTAGTGAGTGGTCTTGCTGATGCATTATTAG |
| | | | | ACAGTGTTCCAATGGTTGCAATAACCGGTCAAGTTCCTAGAAGAAT |
| | | | | GATTGGTACCGATGCGTTTCAAGAAACCCCTATTGTTGAGGTAACA |
| | | | | CGTTCCATTACTAAACATAATTATTTAGTTTTGGATGTTGAAGATATT |
| | | | | CCTAGGGTTGTTAGGGAGGCTTTTTATCTTGCGTCCTCGGGTCGACC |
| | | | | CGGTCCGGTTTTAATTGATGTGCCTAAGGATATACAGCAGCAGTTG |
| | | | | GTAGTGCCTAAATGGGATGAGCCTATGAGGTTACCGGGTTATTTGT |
| | | | | CTCGGTTGCCGAAAACGGAGAATAATGGGCAGTTGGAACAGATTG |
| | | | | TTAGGTTGGTTAGTGAGGCGAAGAGGCCGGTTTTGTATGTAGGGG |
| | | | | GTGGGTGTTTGAATTCGGCGGATGAGTTGAGGCGGTTTGTGGAGT |
| | | | | TAACGGGGATACCGGTTGCGAGTACTTTGATGGGGCTTGGAGCGT |
| | | | | ATCCTGCTTCGAGTGATTTGTCGTTACATATGCTTGGGATGCATGGG |
| | | | | ACGGTTTATGCGAATTATGCGGTGGATAAGAGTGATTTGTTGCTTG |
| | | | | CGTTTGGGGTGAGGTTTGATGATCGTGTGACGGGGAAGCTTGAGG |
| | | | | CGTTTGCTAGTAGGGCGAAGATTGTTCATATTGATATTGATTCAGCG |
| | | | | GAGATTGGGAAGAATAAGCAGCCTCATGTGTCGATTTGTGGTGATA |
| | | | | TTAAGGTCGCGTTACAAGGGCTTAACGAGATTTTGGAGGAAAAGA |
| | | | | ATTCGGTGACTAATCTTGATTTTTCGAATTGGAGGAAGGAGTTGGA |
| | | | | CGAGCAAAAGGTTAAGTTTCCGCTGAGTTTTAAAACGTTTGGTGAA |
| | | | | GCTATTCCTCCGCAGTATGCCATTCAAGTGCTTGATGAGTTAACGGG |
| | | | | TGGGAATGCGATTATTAGCACTGGGGTCGGGCAGCATCAGATGTG |
| | | | | GGCAGCCCAGTTTTACAAATACAACAGACCTAGACAATGGCTGACC |
| | | | | TCGGGTGGACTAGGGGCAATGGGTTTTGGGCTGCCTGCGGCTATT |
| | | | | GGGGCGGCTGTTGCAAGACCTGATGCAGTAGTAGTTGATATCGATG |
| | | | | GCGATGGAAGCTTTATAATGAATGTTCAAGAGTTAGCAACCATCCG |
| | | | | TGTTGAAAACCTGCCTGTTAAGATTATGTTACTTAACAATCAGCATT |
| | | | | TGGGTATGGTGGTTCAGTGGGAGGATCGGTTTTACAAGGCGAATC |
| | | | | GGGCTCATACCTACTTAGGAAATCCGTCAAAAGAGTCTGAAATATT |
| | | | | CCCCAACATGTTGAAGTTTGCTGAAGCGTGTGATATACCGGCTGCC |
| | | | | CGAGTGACCCGAAAGGCAGATCTAAGAGCAGCTATTCAGAAGATG |
| | | | | TTGGATACACCTGGGCCTTACTTGTTGGATGTGATCGTGCCACATCA |
| | | | | AGAACATGTGTTGCCCATGATCCCGGCTGGTGGAGGTTTCATGGAT |
| | | | | GTGATCACCGAAGGCGATGGCAGAACGAAATACTAAGCTTCAAAG |
| | | | | TCGCATCGCATATATAGTGTGTTATGTAAGCAGTTTGTCGGTTTTGA |
| | | | | ATGTTTTGTTGTGTAATTTAGTTTCTGGTTATGAATGTTATGGA |
| 23 | Ambrosia | gDNA | 2540 | GAAGGCGTCACCGACGTCTTCGCGTACCCAGGCGGCGCCTCAGTGG |
| | trifida | Contig | | AGATCCACCAAGCTCTGACCCGCTCCACAACCATCCGAAACGTCCTT |
| | | | | CCCCGTCATGAACAGGGCGGCGTCTTCGCCGCCGAAGGCTACGCAC |
| | | | | GCGCCTCCGGTCTTCCTGGCGTCTGTATTGCTACCTCTGGTCCTGGA |
| | | | | GCTACAAACCTAGTGAGTGGTCTTGCTGATGCATTATTAGACAGTGT |
| | | | | TCCAATGGTTGCAATAACCGGTCAAGTTCCTAGAAGAATGATTGGA |
| | | | | ACCGATGCGTTTCAAGAAACCCCTATTGTTGAGGTAACACGTTCCAT |
| | | | | TACTAAACATAATTATTTAGTTTTGGATGTGGAAGATATTCCTAGGG |
| | | | | TTGTTAGGGAGGCTTTTTATCTTGCGTCTTCGGGTCGGCCCGGTCCG |
| | | | | GTTTTAATTGATGTGCCTAAGGATATACAGCAGCAGTTGGTAGTGC |
| | | | | CTAAATGGGATGAGCCTATGAGGTTACCGGGTTATTTGTCTCGGTT |
| | | | | GCCGAAAACGGAGAATAATGGGCAGTTGGAACAGATTGTTAGGTT |
| | | | | GGTTAGTGAGGCGAAGAGGCCGGTTTTGTATGTAGGGGGTGGGTG |
| | | | | TTTGAATTCGGCGGATGAGTTGAGGCGGTTTGTGGAGTTAACGGG |
| | | | | GATACCGGTTGCGAGTACTTTGATGGGGCTTGGAGCGTATCCTGCT |
| | | | | TCGAGTGATTTGTCGTTACATATGCTTGGGATGCATGGGACGGTTT |
| | | | | ATGCGAATTATGCGGTTGATAAGAGTGATTTGTTGCTTGCGTTTGG |
| | | | | GGTGAGGTTTGATGATCGTGTGACGGGGAAGCTTGAGGCGTTTGC |
| | | | | TAGTAGGGCGAAGATTGTTCATATTGATATTGATTCAGCGGAGATT |
| | | | | GGGAAGAATAAGCAGCCTCATGTGTCGATTTGTGGTGATATTAAGG |
| | | | | TCGCGTTACAAGGGCTTAACGAGATTTTGGAGGAAAAGAATTCGGT |
| | | | | GACTAATCTTGATTTTTCGAATTGGAGGAAGGAGTTGGACGAACAA |
| | | | | AAGGTTAAGTTTCCGCTGAGTTTTAAAACGTTTGGTGAAGCTATTCC |
| | | | | TCCGCAGTATGCCATTCAAGTGCTTGATGAGTTAACGGGTGGGAAT |
| | | | | GCGATTATTAGCACTGGGGTCGGGCAGCATCAGATGTGGGCAGCC |
| | | | | CAGTTTTACAAATACAACAGACCTAGACAATGGCTGACCTCGGGTG |
| | | | | GACTAGGGGCAATGGGTTTTGGGCTGCCTGCGGCTATTGGGGCGG |
| | | | | CTGTTGCAAGACCTGATGCGGTAGTAGTTGATATCGATGGCGATGG |
| | | | | AAGCTTTATAATGAATGTTCAAGAGTTAGCAACCATCCGTGTTGAAA |
| | | | | ACCTGCCTGTTAAGATTATGTTACTTAACAATCAGCATTTGGGTATG |
| | | | | GTGGTTCAGTGGGAGGATCGGTTTTACAAGGCGAATCGGGCTCATA |
| | | | | CCTACTTAGGAAATCCGTCAAAAGAGTCTGAAATATTCCCCAACATG |
| | | | | TTGAAGTTTGCTGAAGCGTGTGATATACCGGCTGCCCGAGTGACCC |
| | | | | GAAAGGCAGATCTAAGAGCAGCTATTCAGAAGATGTTGGATACACC |
| | | | | TGGGCCTTACTTGTTGGATGTGATCGTGCCACATCAAGAACATGTGT |
| | | | | TGCCCATGATCCCGGCTGGTGGAGGTTTCATGGATGTGATCACCGA |
| | | | | AGGCGATGGCAGAACGAAATACTAAGCTTCAAAGTCGCATCGCATA |
| | | | | TATAGTGTGTTATGTAAGCAGTTTGTCGGTTTTGAATGTTTTGTTTTG |
| | | | | TTGTGTAATTTAGTTTCTGGTTATGAATGTTATGGATCAGTTTGTCA |
| | | | | ACGTTTCTTTAATTTAATAGCTTTCATGAATAAATTTCAAAGATTTAT |
| | | | | CTTCTGTTTTATGTTTATATTGAAGACCAAAGTTATGTTTATGTTACA |
| | | | | CACCAGTGAACATGTTTTCTAAAGGTCATATGTTGTTTTCTATAATTC |
| | | | | CAAAAGAGTAGTTTGGTGATCCGTCTCTTTCAGTCCATTTGTGCTCT |
| | | | | CAGTTGTGTAGATGTCGATTATGTATGGTCACTCTGTGCTTTACATT |
| | | | | GTTGTACCAGGTTTGAAGGGTGAGTGTTCCTCCACGTTTCTACCTAA |
| | | | | ATTCTCTCGGCGGCGCTTCCATTTGTGGTGGCGTTCACGGCGGTTCT |
| | | | | TTAACTCATACTTTAACGTTCACCTGATGTAATCAAATGTTAATTACT |
| | | | | AACGCTTTTATGTCTCTTTCCTCTACATCAATTATAACATGTATTTATT |
| | | | | GCCCCCTTTTGTTCTTAGCTTTAATCTATACTATTAATGTATCTGGTA |
| | | | | TTAAGCCTTAGGTATGAAATTTCTTTCCCAAAGCTGTGCATCGCGAA |
| | | | | TGCTTTTAGATTAATATGTTGGTTTATGTATTATGTTTTCAAGGACTA |
| | | | | AATTTACATCTGATGCAATGTATACATGGTTAGCAAGGTTTACACCA |
| | | | | AAACATGCAGACAGATTAATTAAGTAAGATGTTAACCTTGAAAACA |
| | | | | TATGCAATGTTAATACTAACTCAGGTAAGATGTTAATACTAAATTTA |
| | | | | TGTATTATGTTTTCAAGGACTAAATT |
| 24 | Ambrosia | gDNA | 2141 | TCCGAAACGTCCTTCCCCGTCATGAACAGGGCGGCGTCTTCGCCGC |
| | trifida | Contig | | CGAAGGCTACGCACGCGCCTCCGGTCTTCCTGGCGTCTGTATTGCTA |
| | | | | CCTCTGGTCCTGGAGCTACAAACCTAGTGAGTGGTCTTGCTGATGC |
| | | | | ATTATTAGACAGTGTTCCAATGGTTGCAATAACCGGTCAAGTTCCTA |
| | | | | GAAGAATGATTGGTACCGATGCGTTTCAAGAAACCCCTATTGTTGA |
| | | | | GGTAACACGTTCCATTACTAAACATAATTATTTAGTTTTGGATGTTG |
| | | | | AAGATATTCCTAGGGTTGTTAGGGAGGCTTTTTATCTTGCGTCTTCG |
| | | | | GGTCGGCCCGGTCCGGTTTTAATTGATGTGCCTAAGGATATACAGC |
| | | | | AGCAGTTGGTAGTGCCTAAATGGGATGAGCCTATGAGGTTACCGG |
| | | | | GTTATTTGTCTCGGTTGCCGAAAACGGAGAATAATGGGCAGTTGGA |
| | | | | ACAGATTGTTAGGTTGGTTAGTGAGGCGAAGAGGCCGGTTTTGTAT |
| | | | | GTAGGGGGTGGGTGTTTGAATTCGGCGGATGAGTTGAGGCGGTTT |
| | | | | GTGGAGTTAACGGGGATACCGGTTGCGAGTACTTTGATGGGGCTT |
| | | | | GGAGCGTATCCTGCTTCGAGTGATTTGTCGTTACATATGCTTGGGAT |
| | | | | GCATGGGACGGTTTATGCGAATTATGCGGTTGATAAGAGTGATTTG |
| | | | | TTGCTTGCGTTTGGGGTGAGGTTTGATGATCGTGTGACGGGGAAGC |
| | | | | TTGAGGCGTTTGCTAGTAGGGCGAAGATTGTTCATATTGATATTGA |
| | | | | TTCAGCGGAGATTGGGAAGAATAAGCAGCCTCATGTGTCGATTTGT |
| | | | | GGTGATATTAAGGTCGCGTTACAAGGGCTTAACGAGATTTTGGAGG |
| | | | | AAAAGAATTCGGTGACTAATCTTGATTTTTCGAATTGGAGGAAGGA |
| | | | | GTTGGACGAGCAAAAGGTTAAGTTTCCGCTGAGTTTTAAAACGTTT |
| | | | | GGTGAAGCTATTCCTCCGCAGTATGCCATTCAAGTGCTTGATGAGTT |
| | | | | AACGGGTGGGAATGCGATTATTAGCACTGGGGTCGGGCAGCATCA |
| | | | | GATGTGGGCAGCCCAGTTTTACAAATACAACAAACCTAGACAATGG |
| | | | | CTGACCTCGGGTGGACTAGGGGCAATGGGTTTTGGGCTGCCTGCG |
| | | | | GCTATTGGGGCGGCTGTTGCAAGACCTGATGCGGTAGTAGTTGATA |
| | | | | TCGATGGCGATGGAAGCTTTATAATGAATGTTCAAGAGTTAGCAAC |
| | | | | CATCCGTGTTGAAAACCTGCCTGTTAAGATTATGTTACTTAACAATC |
| | | | | AGCATTTGGGTATGGTGGTTCAGTGGGAGGATCGGTTTTGCAAGG |
| | | | | CGAATCGGGCTCATAGCTACTTAGGAAATCCGTCAAAAGAGTCTGA |
| | | | | AATATTCCCCAACATGTTGAAGTTTGCCGAAGCATGTGATATACCGG |
| | | | | CTGCCCGAGTGACCCGAAAGGCAGATCTAAGAGCAGCTATTCAGAA |
| | | | | GATGTTGGATACACCTGGGCCTTACTTGTTGGATGTGATCGTGCCA |
| | | | | CATCAAGAACATGTGTTGCCCATGATCCCGGCTGGTGGAGGTTTCA |
| | | | | TGGATGTGATCACCGAAGGCGATGGCAGAACGAAATACTAAGCTTC |
| | | | | AAAGTCGCATCGCATATATAGTGTGTTATGTAAGCAGTTTGTCGGTT |
| | | | | TTGAATGTTTTGTTGTGTAATTTAGTTTCTGGTTATGAATGTTATGGA |
| | | | | TCAGTTTGTCAACGTTTCTTTAATTTAATAGCTTTCATGAATAAATTT |
| | | | | CAAAGATTTATCTTCTGTTTTATGTTTATATTGAAGACCAAAGTTATG |
| | | | | TTTATGTTAGTGACCAGTGAACATGTTTTCTAAAGGTCATATGTTGT |
| | | | | TTTCTATAATTCCAAGAGTTGTTTGGTGATCCGTCTCTTTCAGTCCAT |
| | | | | TTGTGCTCTCAGTTGTGTAGATGTCGATTATGTATGGTCAATAGATT |
| | | | | GTACTCTGTGCTTTACCAGGTTTGAAGGGTGAGTGTTCCTCCACGTT |
| | | | | TCTACCTAAATTCTCTCGGCGGCGCTTCCATTTGTGGTGGCGGTTTC |
| | | | | TACTCTTAACTCATACTTAAACGTTCACCTGATGTAATCTATACTATT |
| | | | | AATATATCTGGTATTAAGCCTTAGGTATGAAATTTCTTTCCCAAAGC |
| | | | | TGTGCAT |
| 25 | Ambrosia | gDNA | 687 | AAACAAAAGAATCGTCCGATTGTCCATCCTATGTCGTGAAAAAAAA |
| | trifida | Contig | | AAGAATAGAGAAATGGTACTATTGTAAATATATTTGAGCATTCATAT |
| | | | | GGTAGCAGAGCCGCCCATCTGTGTTGCTATTATTTCCTATATCTTCTC |
| | | | | TACTATTCTGTGTTGCTATTATTTTTAGAGGGTCAAAGTATCAAAGG |
| | | | | GTGACGAACCGGTTCGACCTCCGACTACCCGTCAGACCGGCCGGTC |
| | | | | CGCTTCAAAACGGTCTAATATTCTAAACACTGCTTGGTGGAAACTGA |
| | | | | AAATAACATCCACATGTTTTTTTGACACTTCATCATTGACACTTCTTC |
| | | | | TATTATAACCAGATAATAATAATAATCAAACACCAACCCTAGAACAC |
| | | | | ACTCATTCAACAATGGCGGCCATCTCTCCCACAAACCCTTCCCTCACC |
| | | | | ACCAAACCGCCGTCATCTTCCGCCGCCACACCACCACCACGTTCCAC |
| | | | | CTTCCTCCCCCGTTTCACATTCCCAATAACCTCCACTTCCCCAATACG |
| | | | | ACACCGTTTCCACATATCCAACGTTCTCTCCGACCACAAACCCACCAT |
| | | | | AACCCATTCCCCATCACCAACCGACCCATTCATCTCCCGTTACGCTCC |
| | | | | AGACCAGCCCCGTAAAGGCGCCGACGTCCTAGTCGAAGCTTTGGAA |
| | | | | CGTGAAGGCGTCACCGACGTCTTCGC |
| 26 | Ambrosia | gDNA | 322 | CCCTAGAACACACTCATTCAACAATGGCAGCCATCTCTCCCACAAAC |
| | trifida | Contig | | CCTTCCTTCACCACCAAACCGCCGTCATCTTCCGCCGCCACACCACCA |
| | | | | CCACGTTCCACCTTCCTCCCCCGTTTCACATTCCCAATAACCTCCACTT |
| | | | | CCCCAATACGACACCGTTTCCACATATCCAACGTTCTCTCCGACCAC |
| | | | | AAACCCACCATAACCCATTCCCCATCACCAACCGACCCATTCATCTCC |
| | | | | CGTTACGCTCCAGACCAGCCCCGTAAAGGCGCCGACGTCCTAGTCG |
| | | | | AAGCTTTGGAACGTGAAGGCGTCACCGACGTCTTCGC |
| 27 | Ambrosia | gDNA | 214 | GACCGACGTCTTCGCCTACCCAGACGGCGCCTCAATGGAGATACAC |
| | trifida | Contig | | CAACTTCTCACCCGCTCCACCACTATCCGAAACGTCCTCCGGTCACA |
| | | | | TCCTGAGATCGTGAACGGGTAAATATTTGATTTTCTTTATTATAATCA |
| | | | | TGTTATATAATAATTATATCATTTAAATAACATCGACTGATTCTCTAT |
| | | | | TCGTATGTTTTCCACTTGATCTATG |
| 28 | Chenopodium | cDNA | 2572 | CTGCTTGGTCTGGACCATATGTCAGAACATTTACTCTCACTATTGTCA |
| | album | Contig | | TTACTCACTACACCAGAGGCCACCATCATCAGAGTCCGTTCAACATC |
| | | | | ACCACTCTCCTGTCTAACACCTACTCCTTCTCTGTAGCTATAATCAGG |
| | | | | AACGTTTTGATCTGCCTGGTAATGAAGGAATTGGGAGCCAGAATGA |
| | | | | ACCTGAAGATTAGACTGGGTTGAATCAATCATGGAAGATCCAAATA |
| | | | | ACCCATGTTCAAAAATCCTAAAAACACCCAAAAACAAACAAACCCCA |
| | | | | CAAAATAGAAAGAACATCCATTGACCTAATTTCTCTCTCTTCAACCTT |
| | | | | CAATGCCTTCATCAATGGCGGCTACTTCCTCAAACCCTTCATTCTCCC |
| | | | | CTTTCCCAACCCTCTCTTCCAAAACCCCTAAACCCCAATCCTCCATTT |
| | | | | ACTCCCTCCCTTTTCCCACTAACCCCAAAACGCCATCTTCCTTCCTCC |
| | | | | GCCGCCCTCTCCAAATCTCCGCCTCTCAATCCGCTAACCCAAAACCG |
| | | | | CCATCCGCTACTCAAACCGCTGTTCCTTCCCCTCTCACCGAGGAAAA |
| | | | | CCCGCAATCTTTCGTTTCCCGATTTGCCCCTGATGAACCCAGAAAAG |
| | | | | GCTGTGATGTCCTCGTTGAAGCTTTGGAGCGTGAAGGTGTTACCAA |
| | | | | TGTGTTTGCTTACCCTGGTGGTGCATCAATGGAGATTCATCAAGCTC |
| | | | | TCACGCGCTCTGGTTCTATCAGGAACGTCCTCCCCCGGCACGAGCA |
| | | | | AGGTGGGGTTTTCGCTGCGGAAGGGTATGCTCGAGCTACTGGGCG |
| | | | | CGTGGGTGTGTGCATTGCAACCTCTGGTCCTGGTGCCACTAATCTTG |
| | | | | TATCGGGTCTCGCTGATGCGCTGCTTGATTCTGTCCCTTTGGTTGCG |
| | | | | ATCACTGGTCAGGTGCCTCGCCGAATGATTGGAACTGATGCATTTC |
| | | | | AGGAGACTCCTATTGTTGAGGTTACTCGTTCTATCACTAAGCATAAT |
| | | | | TATTTGGTTTTAGATGTTGAGGATATTCCTAGAGTTGTTAAGGAAGC |
| | | | | GTTTTTATTTAGCTAATTCTGGTAGGCCTGGACCTGTTTTGATTGATA |
| | | | | TTCCTAAAGATATTCAGCAACAATTGGTTGTGCCTAATTGGGATCAG |
| | | | | CCTATTAAGCTTGGTGGGTATGTTTCTAGGCTGCCAAAATCGAAGTT |
| | | | | TTCGGCGAATGAGGAAGGGCTTCTTGAGCAGATTGTGAGGTTGAT |
| | | | | GAGTGAGGCTAAGAAGCCTGTGTTGTATGTGGGAGGTGGGTGTTT |
| | | | | GAATTCTAGTGAGGAATTGAGGAAATTCGTCGAATTGACAGGGATT |
| | | | | CCGGTGGCTAGTACTTTGATGGGGTTAGGAGCTTACCCTTGTAATG |
| | | | | ATGAATTGTCCCTTCATATGTTGGGAATGCACGGGACTGTGTACGC |
| | | | | GAATTATGCTGTTGATAAGGCGGATTTGCTGCTTGCTTTCGGGGTTA |
| | | | | GGTTTGATGATCGTGTGACAGGGAAGCTCGAGGCGTTTGCTAGCCG |
| | | | | AGCTAAGATTGTGCACATTGATATTGATTCTGCTGAGATTGGGAAG |
| | | | | AATAAGCAGCCTCATGTGTCGATTTGTGCTGATGTTAAGCTGGCGTT |
| | | | | GAAGGGTATGAATAAGATTCTGGAGTCTCGAAAAGGGAAGTTGAA |
| | | | | TTTAGATTACTCTAGTTGGAGGGAGGAGTTGGGTGAGCAGAAGAA |
| | | | | GAAGTTTCCATTGAGTTTTAAGACCTTTGGTGAGGCTATTCCTCCCC |
| | | | | AATACGCCATTCAGATGCTCGATGAGCTAACTGATGGTAATGCTGTT |
| | | | | ATAAGTACTGGTGTTGGGCAACATCAAATGTGGGCTGCCCAGCATT |
| | | | | ACAAGTATCGAAACCCTCGACAGTGGCTGACCTCAGGTGGTTTAGG |
| | | | | AGCCATGGGATTCGGGCTACCAGCTGCCATTGGAGCAGCTGTGGCT |
| | | | | CGTCCAGAGTCAGTGGTTGTCGATATTGATGGGGATGGGAGTTTCA |
| | | | | TCATGAATGTGCAAGAATTAGCTACCATAAGGGTGGAAAATCTGCC |
| | | | | TGTTAAGATAATGCTCTTAAACAATCAACATTTAGGTATGGTGGTTC |
| | | | | AATGGGAAGACAGATTCTACAAAGCCAATAGAGCGCATACATACCT |
| | | | | TGGAAACCCTTCTAAAGAGTCTGAAATCTTCCCCGATATGCTCAAAT |
| | | | | TCGCTGAAGCCTGTGATATTCCTGCTGCCCGTGTTACCAAGGTGAGC |
| | | | | GAGTTGAGGGCCGCAATGCAGAAAATGTTGGATACTCCGGGGCCA |
| | | | | TACCTGCTGGATGTGATTGTACCTCATCAAGAGCATGTGCTGCCTAT |
| | | | | GATCCCAAGTGGTGCCGCCTTCAAAGATATCATTAACGAGGGTGAT |
| | | | | GGAAGAACATCTTATTAATCGCATTATCGATGATGATGCTTAAATCT |
| | | | | GTATGTATAATAATATGTTTAGAGAGGTTATGACTGTTTTCTCTTTA |
| | | | | GGTTAGTTTGTTGTTCACATGCTGCTTGCTACTACTATGATTAACTTT |
| | | | | TTTGTTGTTTTTGAGGTATTACTATGGATGATTAGCTCAAAGATTTC |
| | | | | ACAAAAGTGGTCTTTGAGCTATGTATGTTCTCTTGCGAAATACACTC |
| | | | | GTTGGCTC |
| 29 | Chenopodium | cDNA | 2103 | CCTCCGCCGCCCTCTCCAAATCTCCGCCTCTCAATCCGCTAACCCCAA |
| | album | Contig | | ACCTCCATCCGCTACTCAAACCGCTGTTCCATCCCCTCTCACTGACGA |
| | | | | AAACCCCCAATCTTTCGTTTCCCGATTTGCCCCTGATGAACCCAGAA |
| | | | | AAGGGTGTGATGTTCTTGTTGAAGCTTTGGAGCGTGAAGGTGTTAC |
| | | | | CAATGTGTTTGCTTACCCTGGTGGTGCATCAATGGAGATCCATCAAG |
| | | | | CTCTCACGCGCTCTACCTCGATCAGGAACGTCCTCCCCCGGCACGAG |
| | | | | CAAGGTGGGGTTTTCGCTGCGGAAGGGTATGCTCGAGCTACTGGG |
| | | | | CGCGTGGGTGTGTGCATTGCAACCTCTGGTCCTGGTGCCACTAATCT |
| | | | | TGTATCGGGTCTCGCTGATGCGCTGCTTGATTCTGTCCCTTTGGTTG |
| | | | | CGATCACTGGTCAGGTGCCTCGCCGAATGATTGGAACTGATGCATT |
| | | | | TCAGGAGACTCCTATTGTTGAGGTTACTCGTTCTATCACTAAGCATA |
| | | | | ATTATTTGGTTTTAGATGTTGAGGATATTCCTAGAGTTGTTAAGGAA |
| | | | | GCGTTTTTATTTAGCTAATTCTGGTAGGCCTGGACCTGTTTTGATTG |
| | | | | ATATTCCTAAAGATATTCAGCAACAATTGGTTGTGCCTAATTGGGAT |
| | | | | CAGCCTATTAAGCTTGGTGGGTATGTTTCTAGGCTGCCAAAATCGA |
| | | | | AGTTTTCGGCGAATGAGGAAGGGCTTCTTGAGCAGATTGTGAGGTT |
| | | | | GATGAGTGAGGCTAAGAAGCCTGTGTTGTATGTGGGAGGTGGGTG |
| | | | | TTTGAATTCTAGTGAGGAATTGAGGAAATTCGTCGAATTGACAGGG |
| | | | | ATTCCGGTGGCTAGTACTTTGATGGGGTTAGGAGCTTACCCTTGTA |
| | | | | ATGATGAATTGTCCCTTCATATGTTGGGAATGCACGGGACTGTGTA |
| | | | | CGCGAATTATGCTGTTGATAAGGCGGATTTGCTGCTTGCTTTCGGG |
| | | | | GTTAGGTTTGATGATCGTGTGACAGGGAAGCTCGAGGCGTTTGCTA |
| | | | | GCCGAGCTAAGATTGTGCACATTGATATTGATTCTGCTGAGATTGG |
| | | | | GAAGAATAAGCAGCCTCATGTGTCGATTTGTGCTGATGTTAAGCTG |
| | | | | GCGTTGAAGGGTATGAATAAGATTCTGGAGTCTCGAAAAGGGAAG |
| | | | | TTGAATTTAGATTACTCTAGTTGGAGGGAGGAGTTGGGTGAGCAGA |
| | | | | AGAAGAAGTTTCCATTGAGTTTTAAGACCTTTGGTGAGGCTATTCCT |
| | | | | CCCCAATACGCCATTCAGATGCTCGATGAGCTAACTGATGGTAATG |
| | | | | CTGTTATAAGTACTGGTGTTGGGCAACATCAAATGTGGGCTGCCCA |
| | | | | GCATTACAAGTATCGAAACCCTCGACAGTGGCTGACCTCAGGTGGT |
| | | | | TTAGGAGCCATGGGATTCGGGCTACCAGCTGCCATTGGAGCAGCTG |
| | | | | TGGCTCGTCCAGAGTCAGTGGTTGTCGATATTGATGGGGATGGGA |
| | | | | GTTTCATCATGAATGTGCAAGAATTAGCTACCATAAGGGTGGAAAA |
| | | | | TCTGCCTGTTAAGATAATGCTCTTAAACAATCAACATTTAGGTATGG |
| | | | | TGGTTCAATGGGAAGACAGATTCTACAAAGCCAATAGAGCGCATAC |
| | | | | ATACCTTGGAAACCCTTCTAAAGAGTCTGAAATCTTCCCCGATATGC |
| | | | | TCAAATTCGCTGAAGCCTGTGATATTCCTGCTGCCCGTGTTACCAAG |
| | | | | GTGAGCGAGTTGAGGGCCGCAATGCAGAAAATGTTGGATACTCCG |
| | | | | GGGCCATACCTGCTGGATGTGATTGTACCTCATCAAGAGCATGTGC |
| | | | | TGCCTATGATCCCAAGTGGTGCCGCCTTCAAAGATATCATTAACGAG |
| | | | | GGTGATGGAAGAACATCTTATTAATCGCATTATCGATGATGATGCTT |
| | | | | AAATCTGTATGTATAATAATATGTTTAGAGAGGTTATGACTGTTTTC |
| | | | | TCTTTAGGTTAGTTTGTTGTTCACATGCTGCTTGCTACTACTATGATT |
| | | | | AACTTTTTTGTTGTTTTTGAGGTATTACTATGGATGATTAGCTCAAA |
| | | | | GATTTCACAAAAGTGGTCTTTGAGCTATGTATGTTCTCTTGCGAAAT |
| | | | | ACACTCGTTGGCTC |
| 30 | Conyza | cDNA | 2379 | CGTGTTGTGGTCATACATCACCACGACGTCTGTGATGGTACCGAATT |
| | canadensis | Contig | | GATCAAAGTAATTCTTGAAATCACTTCCACACACACTACTCCTAGCT |
| | | | | CTCATTTTTTATTTCCTTTTTCATCCCCAGCAAAAACACGCACACATA |
| | | | | AATTAAATTCAAACGAATCAAACCCTAAAAACAACAAAAAAAAATA |
| | | | | ATACAATCTCACACACACATTTTCAAACATGGCGGCCGTAACTTCAC |
| | | | | CAAACCCACCTTTCACCACCACCACCACAAAACCACCGCATTCCGCC |
| | | | | ACCACATTTCACCGTCCCACCACCACTCATTTATCCTTCCCAACCCCA |
| | | | | CCAAAACGAAACCGTTTCCTTCACATCACCAACGTCCTCTCGGACCA |
| | | | | CAAACCCATCACTACCACCACTACAACCACCGTTCCACCACCTTCATT |
| | | | | CACTTCCCGTTTCGCCGCCGACCAACCCCGGAAGGGCTCCGACGTTC |
| | | | | TCGTAGAAGCCCTCGAACGTGAAGGCGTCACCGACGTCTTCGCTTA |
| | | | | CCCTGGTGGCGCGTCAATGGAGATCCACCAAGCTCTCACGCGCTCC |
| | | | | ACCACCATCCGCAACGTCCTCCCCCGCCATGAACAAGGTGGCATCTT |
| | | | | CGCCGCAGAAGGCTACGCACGTGCCTCCGGTCTCCCCGGCGTCTGT |
| | | | | ATAGCCACTTCCGGTCCCGGCGCCACAAACCTTGTCTCCGGCCTTGC |
| | | | | TGACGCGCTTCTTGACAGTGTCCCCGTCGTTGCCATCACCGGCCAAG |
| | | | | TTCCCCGGCGAATGATCGGAACTGATGCTTTTCAAGAAACACCCATT |
| | | | | GTTGAGGTAACAAGATCCATTACTAAACATAACTATTTGGTTTTAGA |
| | | | | TGTTGATGATATTCCAAGAATTGTTAGAGAAGCTTTTTATCTTGCCC |
| | | | | GGTCGGGTCGACCCGGACCCGTTTTGATTGATATTCCAAAAGATAT |
| | | | | ACAACAACAGTTATGTGTGCCTAAATGGGATGAGCCCATGAGGCTT |
| | | | | CCTGGTTATTTATCTAGGTTACCTAAGCCACCTAATGATGGTTTACTA |
| | | | | GAACAAATTGTGAGGTTAGTTGGAGAGTCGAAAAGGCCGGTTTTGT |
| | | | | ATGTAGGTGGTGGTTGTTTGAATTCGAGTGATGAGTTGAGACGGTT |
| | | | | TGTTGAGCTTACGGGTATTCCGGTAGCTAGTACTTTAATGGGTCTTG |
| | | | | GGTCTTATCCAGCTTCGAGTGATTTGTCTCTTCAAATGCTTGGAATG |
| | | | | CATGGAACTGTTTATGCGAATTATGCTGTTGATAAGGCGGATTTGTT |
| | | | | GCTTGCGTTTGGGGTTAGGTTTGATGACCGTGTGACTGGAAAGCTT |
| | | | | GAAGCTTTTGCTAGTAGAGCTAAGATTGTTCATATTGATATTGACTC |
| | | | | TGCTGAGATTGGGAAGAATAAGCAGCCACATGTGTCGGTTTGTGGT |
| | | | | GATATTAAGATTGCGTTACAGGGTCTTAACAAGATTTTAGAAGGGA |
| | | | | GGCGTGAGATGAGTAATCTTGATTTTTCGGCGTGGAGGGCAGAGTT |
| | | | | GGATGAACAAAAGGTGAACCATCCGTTGAGTTTTAAAACGTTCGGT |
| | | | | GAAGCTATTCCACCGCAATATGCTATTCAGGTGCTTGATGAGTTAAC |
| | | | | GGGTGGGAATGCTATTATAAGTACTGGGGTCGGGCAACATCAGAT |
| | | | | GTGGGCTGCTCAGTTTTACAAGTACAACAGACCTAGACAGTGGTTG |
| | | | | ACTTCAGGTGGACTAGGGGCTATGGGCTTTGGACTGCCTGCAGCCA |
| | | | | TTGGTGCAGCCGTTGCAAGACCTGATGCAGTGGTAGTGGATATCGA |
| | | | | TGGTGATGGAAGTTTTATCATGAATGTTCAAGAGTTGGCTACTATCC |
| | | | | GAGTGGAAAATCTTCCTGTCAAGGTTTTGTTACTCAATAATCAGCAT |
| | | | | TTGGGTATGGTGGTTCAATGGGAGGATCGTTTCTACAAGGCAAATA |
| | | | | GGGCGCATACCTACTTGGGGAACCCATCAAAGGAGGCTGAAATATT |
| | | | | TCCAAACATGTTGAAGTTTGCCGAAGCGTGTGATATACCAGCTGCA |
| | | | | CGAGTGACCCGTAAAGCTGATCTACGGGCGGCTATTCAGAAGATGT |
| | | | | TGGATACTCCTGGGCCTTACTTGTTGGATGTCATTGTGCCGCATCAA |
| | | | | GAACATGTTTTGCCGATGATTCCTGCTGGTGGGGGCTTCATGGATG |
| | | | | TGATCACCGAGGGTGATGGTAGAACGAAGTATTGAGCTTCAAAGTC |
| | | | | ACAACTTAGTTTCAAGTTTCAACCACTGTATGTAAACATCTAGCATCT |
| | | | | TTACTTAAGAGGTGTGAGCAGTTTGTTGGATTTGAAGTTAATTTCTT |
| | | | | GTTGTGTATTTTAGTTTCTGGTTTTTTGAATGTTCCGTAGTAGTGTAT |
| | | | | TGACTGTTCTTTTAGTAGCCTCATGAATAAATGTCTTTTTTTCCTTG |
| 31 | Conyza | gDNA | 5989 | CAAAATTAAAATTTAAAAGTACTGGATGTTCAAAGTTAACAAACAA |
| | canadensis | Contig | | GATAAAAATAATATGTGACTTGATAAACCATATATTGTTGTCTATCG |
| | | | | AGGGTGATCGACCTGCATCTTCATATCCCTAGTGTTGTGTCTCGACT |
| | | | | TTTTGCATGTGTCATCATTGTCAAGAACCAATTCTATTGTATCATAAC |
| | | | | GTTTACACTAGCTAATCAACCCAGGTTCTACCTGAGCATTTTATTAA |
| | | | | AATTTTAAAAGCAAAAAAAATTTTAAAAAGTGTATATAATTTTTGTT |
| | | | | ATTGATATATTATAACTCGGTTTTGAGATATTAAATTAATTAACATG |
| | | | | ATAATTTATATATATTAGTATATATTGCATTAACAAAACATAAAAAG |
| | | | | ACATTTTATAATATTATATTAAAAAAAATTTACTTTGATTCGAATAAA |
| | | | | AGAATAAATTTGTAGACATCATAAATAAAAGAAAACATTTTGAAAA |
| | | | | TATTTAATGGGCTATTTATCTTTAAAAAGATTTTTGATTAAATATGAC |
| | | | | ATTATAAATAGATAAAACATTTTAAAGAAATTTGTAAACATGACACA |
| | | | | TCATAATTGTTTTTAAAAATAATTTAAAAAGAATCCTTCTTTATTATA |
| | | | | TATAAAGATTACATATTAAATATATGTTGTTTCAATCTTACCTTTCTTT |
| | | | | TTAAATAAACTTAGCTAACTTTGATAGTTTGTTTTTTATTTTTTTTATT |
| | | | | TTTAAACAACAGATTGATTTTATTAATAGTTGGTTGAAAATTGATCA |
| | | | | ACCAATAAAAAGAAAAAATTAAATACAAAGAAGAGGACTTTTCAAC |
| | | | | CATTCATCCCAAGATATTTGCTTCTTTTTATAGCGATACCATAAAAAT |
| | | | | GAATGAGTTATAATCGAATCAACAAGAACATCATGTCTTTTATTGGA |
| | | | | GCAACCGAAGACTTTATAACTTTGATAGTTGAGTTGCGAAAATTTAT |
| | | | | TTAAAGTTCGACTGGGATATTTCTAATCTACTAGCTACAAACACTTG |
| | | | | AGCTAAAATTGAACCTTGAACATGGTAAGTAAATCATTTTGGAGAG |
| | | | | ATTTCATGCTTTCTCTGTATCTTAATTCTATTTGTCTAATTGGTTTGGT |
| | | | | CGCAACCCGAAACATGGTTAGAATTACATAAAAAACACAACGAAGT |
| | | | | GAAATCCGGTAGCTCGACCTTCGTTCGTTGGGAGTTTTTCCTCGTGT |
| | | | | CTTGGGTCACTGTAATTGGCACGAGGTTTTTTCACTCCTTTTGTAGTT |
| | | | | CTTACAGTTAATATTTCGGAAAGAGTCTAAATTTATTGTATTGCTTTT |
| | | | | TTTAGATTGTAATTATAATTATACCCTATTACAACTAGTGCCTTGTTA |
| | | | | GTTGTTTATTTTAAATAATATTTTCTGTCGTTCTAGGAAAAAAAAAAT |
| | | | | TGAACCTTGAACACTTAATGCGGAAGTAGTTAATACTTGCAAACTTA |
| | | | | AGGAATAAGTTAAGTAGGCTTTGATGCTATAGATAGTCCTAAGCCA |
| | | | | TCACTCTTCAAAACACAATTTACACAATATTTCTAGAAAAATCAAAA |
| | | | | CTTCCATAGATATGTGAAAATAATAAAATGGATGTATATACAAAAA |
| | | | | AAAAAACATAATAAAAAATTGAAAAACTTATACACGCGTACAAGCC |
| | | | | AATGATTATAGTCTAACTGGTTAGAGACATTTTAGGTTTATCTAAAG |
| | | | | TTTTGAGTCGATCTTTAGGAATGATAAGTTTAGGTTTTTTTTTTCTCT |
| | | | | GAATATTTATAACGGTCCATGGTCAACATTTTGTTTTCTAAGATACG |
| | | | | TGCAAGACTTACCATTATATGGTGATGTTTCTCTAATGTCACATACC |
| | | | | GACCAAATGTTTGTACAAAAATAAAAATAAATACGTGTATAAAAGT |
| | | | | CGGCAAACGAAAATTAAAAAAACAGGATCTCATACATTCACTTCCAC |
| | | | | ACACACTACTCCTAGCTCTCATTTTTTATTTCCTTTTTCATCCCCAGCA |
| | | | | AAAACACGCACACATAAATTAAATTCAAACGAATCAAACCCTAAAA |
| | | | | ACAACAAAAAAAAAATAATACAATCTCACACACACATTTTCAAACAT |
| | | | | GGCGGCCGTAACTTCACCAAACCCACCTTTCACCACCACCACCACAA |
| | | | | AACCACCGCATTCCGCCACCACATTTCACCGTCCCACCACCACTCATT |
| | | | | TATCCTTCCCAACCCCACCAAAACGAAACCGTTTCCTTCACATCACCA |
| | | | | ACGTCCTCTCGGACCACAAACCCATCACTACCACCACTACAACCACC |
| | | | | GTTCCACCACCTTCATTCACTTCCCGTTTCGCCGCCGACCAACCCCGG |
| | | | | AAGGGCTCCGACGTTCTCGTAGAAGCCCTCGAACGTGAAGGCGTCA |
| | | | | CCGACGTCTTCGCTTACCCTGGTGGCGCGTCAATGGAGATCCACCA |
| | | | | AGCTCTCACGCGCTCCACCACCATCCGCAACGTCCTCCCCCGCCATG |
| | | | | AACAAGGTGGCATCTTCGCCGCAGAAGGCTACGCACGTGCCTCCGG |
| | | | | TCTCCCCGGCGTCTGTATAGCCACTTCCGGTCCCGGCGCCACAAACC |
| | | | | TTGTCTCCGGCCTTGCTGACGCGCTTCTTGACAGTGTCCCCGTCGTT |
| | | | | GCCATCACCGGCCAAGTTCCCCGGCGAATGATCGGAACTGATGCTT |
| | | | | TTCAAGAAACACCCATTGTTGAGGTAACAAGATCCATTACTAAACAT |
| | | | | AACTATTTGGTTTTAGATGTTGATGATATTCCAAGAATTGTTAGAGA |
| | | | | AGCTTTTTATCTTGCCCGGTCGGGTCGACCCGGACCCGTTTTGATTG |
| | | | | ATATTCCAAAAGATATACAACAACAGTTATGTGTGCCTAAATGGGA |
| | | | | TGAGCCCATGAGGCTTCCTGGTTATTTATCTAGGTTACCTAAGCCAC |
| | | | | CTAATGATGGTTTACTAGAACAAATTGTGAGGTTAGTTGGAGAGTC |
| | | | | GAAAAGGCCGGTTTTGTATGTAGGTGGTGGTTGTTTGAATTCGAGT |
| | | | | GATGAGTTGAGACGGTTTGTTGAGCTTACGGGTATTCCGGTAGCTA |
| | | | | GTACTTTAATGGGTCTTGGGTCTTATCCAGCTTCGAGTGATTTGTCT |
| | | | | CTTCAAATGCTTGGAATGCATGGAACTGTTTATGCGAATTATGCTGT |
| | | | | TGATAAGGCGGATTTGTTGCTTGCGTTTGGGGTTAGGTTTGATGAC |
| | | | | CGTGTGACTGGAAAGCTTGAAGCTTTTGCTAGTAGAGCTAAGATTG |
| | | | | TTCATATTGATATTGACTCTGCTGAGATTGGGAAGAATAAGCAGCC |
| | | | | ACATGTGTCGGTTTGTGGTGATATTAAGATTGCGTTACAGGGTCTTA |
| | | | | ACAAGATTTTAGAAGGGAGGCGTGAGATGAGTAATCTTGATTTTTC |
| | | | | GGCGTGGAGGGCAGAGTTGGATGAACAAAAGGTGAACCATCCGTT |
| | | | | GAGTTTTAAAACGTTCGGTGAAGCTATTCCACCGCAATATGCTATTC |
| | | | | AGGTGCTTGATGAGTTAACGGGTGGGAATGCTATTATAAGTACTGG |
| | | | | GGTCGGGCAACATCAGATGTGGGCTGCTCAGTTTTACAAGTACAAC |
| | | | | AGACCTAGACAGTGGTTGACTTCAGGTGGACTAGGGGCTATGGGC |
| | | | | TTTGGACTGCCTGCAGCCATTGGTGCAGCCGTTGCAAGACCTGATG |
| | | | | CAGTGGTAGTGGATATCGATGGTGATGGAAGTTTTATCATGAATGT |
| | | | | TCAAGAGTTGGCTACTATCCGAGTGGAAAATCTTCCTGTCAAGGTTT |
| | | | | TGTTACTCAATAATCAGCATTTGGGTATGGTGGTTCAATGGGAGGA |
| | | | | TCGTTTCTACAAGGCAAATAGGGCGCATACCTACTTGGGGAACCCA |
| | | | | TCAAAGGAGGCTGAAATATTTCCAAACATGTTGAAGTTTGCCGAAG |
| | | | | CGTGTGATATACCAGCTGCACGAGTGACCCGTAAAGCTGATCTACG |
| | | | | GGCGGCTATTCAGAAGATGTTGGATACTCCTGGGCCTTACTTGTTG |
| | | | | GATGTCATTGTGCCGCATCAAGAACATGTTTTGCCGATGATTCCTGC |
| | | | | TGGTGGGGGCTTCATGGATGTGATCACCGAGGGTGATGGTAGAAC |
| | | | | GAAGTATTGAGCTTCAAAGTCACAACTTAGTTTCAAGTTTCAACCAC |
| | | | | TGTATGTAAACATCTAGCATCTTTACTTAAGAGGTGTGAGCAGTTTG |
| | | | | TTGGATTTGAAGTTAATTTCTTGTTGTGTATTTTAGTTTCTGGTTTTTT |
| | | | | GAATGTTCCGTAGTAGTGTATTGACTGTTCTTTTAGTAGCCTCATGA |
| | | | | ATAAATGTCTTTTTTTCCTTGCCTGCGTAGTTTTGTAGAAGTAATCTT |
| | | | | CTGTTTCTATGTTTATAGCAAGGACCAAAAGAAGAGTATTAGAGCC |
| | | | | CCTGGTCTAAAAGTCGGATATCTGAAGTCAAATTTTAGCCCCTATTA |
| | | | | AGATATGTCTCTTTTTGATTGTGGTCAATACTAGCTGTAAGATACCA |
| | | | | TTTGAGATCAGCAATTACCCGTTGCGGTTTCATTTTGGTTTGGTCTG |
| | | | | GTCTGGTCTGGTCTGGTCTATTTCTTGTTGTGTAGTTTCTGCTATTTG |
| | | | | TGGGCTATATGTTCTGGTTTTCTTGGGTTACTGTGTTTGGGCTTAGT |
| | | | | ATTACAATCTATTAGTTTTGAAGTTGGTTCATATTTAGTAAACTATAA |
| | | | | ATTTTCAAAAACTATTCTTGATCGGTCCAGCATCAGTATATACTGGTT |
| | | | | CAGAAACTTGATAGATGTTGCTCAATCTTTCCGTCCTGAATGGGGCA |
| | | | | TTGATTGTTAGGATGTTTTTGTATAGGGTGACTAATATTTCTGGAAG |
| | | | | GATGTTCGGTTTGGTAGTGTTTCCGAACAGGTGTAGGCTGCTTCTG |
| | | | | AGTGGGCAGAGTACGTAATCTCATCACCTACTCCTTGGGGAGGGTT |
| | | | | TGACTTGATTTTGAGTCAAGGGTATAAGTCTTGCAATTGTAGTTTGC |
| | | | | GTTAGGCAGTGGTCAATGATTTATTACTAACTTAGCGTTATATTTTCC |
| | | | | AGCTACAAGTGCTATAAGTTAATGATTTAGTACTAACACAGAATGTA |
| | | | | GTTCATTTATGCATCAACGTAATTATTTTCCACACTAAATTTTTGTGT |
| | | | | GTGAGATGACAATACCGGATGATGAAACTTTTTATACTAAGCAAAG |
| | | | | TGAGTTTCTTGTTTGATCGAATGACTATGATTTATATATCACTTCCCA |
| | | | | AATAGAACGCAATTAGGTGGTTTTAAGATTGGAGTAGAGCAGACCA |
| | | | | TCTTCAAGAACTAGTCCAGAGCGAGTTGGATACTGGGAGTATACAT |
| | | | | CTTTTGGGGTACAGTTTTGATGGCGTTTATTAATATTAATTTCAACAA |
| | | | | ACCATTTTACATGCTAAAAATAGTGGAGTACCTCAAGTTAAAACAGC |
| | | | | TCAAACAGGTTAAAATACATCTAAGTTTAAGGCACAAAACTGTCTAA |
| | | | | AAACTGTAAACATCTGTATTGCTCTAAAAAAAACTCCGATTTAAGGC |
| | | | | ACTTGTGGCACTGCTGTAGTTTCAAACGATTTCCTCCTAACAAGTAA |
| | | | | GCACGTAATATTTGAAGAACGAGTGCTCATGTTTTCAGGACTGCAT |
| | | | | AACACAAAGTCGGGCCTGTGGAGTGCTAAGTGGAGGCGATCTTCG |
| | | | | CCCATAACCATTGCAGTTGCATCAAGCAAAACATGCCATTGGTTTCT |
| | | | | ATGGGCTTCGGACACCCAATGCATTGAGTAGTGGGTCCCATTTACA |
| | | | | TTCGCAGGATAAGAGAACAAGCCTTTTGGGCTTTGCTTACTTTTCCT |
| | | | | TCTAAAATATTGACTGAGTTGTGAACCTTTGATCCTCAGATCTAACC |
| | | | | ATGTCTCGGGTGCTGATATCACTTTTGCGTCCTTGTAACCTGCAAAC |
| | | | | GCTTTTATGTAGTCATGTTCTTCGTTTATGATTGTCATGTAGTAATTA |
| | | | | CCCCTGAAAAATGGGTAACTTTCACCCACCAACATCATGGCATCACG |
| | | | | ATAGCTTGAGGTGAATAAAACAAGATACTCTTCATCAGGCAACCCA |
| | | | | CACTGCTTCAAAACTTTGTTTCTTGCTTGAATTTCAGGTATTGAGATG |
| | | | | AAGCTTCCAAGATATGATGACTTTTTAGTAAGGATATCCAGTAATCT |
| | | | | TGATGGCTCGAGCTGAATTCTTACCAGATCATGGA |
| 32 | Conyza | gDNA | 5989 | CACAAAATTAAAATTTAGAAGTACTGGATGTTCAAAGTTAACAAAC |
| | canadensis | Contig | | AAGATAAAAATAATATGTGACTTGATAAACCATATATTGTTGTCTAT |
| | | | | CGAGGGTGATCGACCTGCATCTTCATATCCCTAGTGTTGTGTCTCGA |
| | | | | CTTTTTGCATGTGTCATCATTGTCAAGAACCAATTCTATTGTATCATA |
| | | | | ACGTTTACACTAGCTAATCAACCCAGGTTCTACCTGAGCATTTTATTA |
| | | | | AAATTTTAAAAGCAAAAAAAATTTTAAAAAGTGTATATAATTTTTGT |
| | | | | TATTGATATATTATAACTCGGTTTTGAGATATTAAATTAATTAACATG |
| | | | | ATAATTTATATATATTAGTATATATTGCATTAACAAAACATAAAAAG |
| | | | | ACATTTTATAATATTATATTAAAAAAAATTTACTTTGATTCGAATAAA |
| | | | | AGAATAAATTTGTAGACATCATAAATAAAAGAAAACATTTTGAAAA |
| | | | | TATTTAATGGGCTATTTATCTTTAAAAAGATTTTTGATTAAATATGAC |
| | | | | ATCATAAATAAATAAAACATTTTAAAGAAATTTGTAAACATGACACA |
| | | | | TCATAATTGTTTTTAAAAATAATTTAAAAAGAATCCTTCTTTATTATA |
| | | | | TATAAAGATTACATATTAAATATATGTTGTTTCAATCTTACCTTTCTTT |
| | | | | TTAAATAAACTTAGCTAACTTTGATAGTTTGTTTTTTATTTTTTTTATT |
| | | | | TTTAAACAACAGATTGATTTTATTAATAGTTGGTTGAAAATTGATCA |
| | | | | ACCAATAAAAAGAAAAAATTAAATACAAAGAAGAGGACTTTTCAAC |
| | | | | CATTCATCCCAAGATATTTGCTTCTTTTTATAGCGATACCATAAAAAT |
| | | | | GAATGAGTTATAATCGAATCAACAAGAACATCATGTCTTTTATTGGA |
| | | | | GCAACCGAAGACTTTATAACTTTGATAGTTGAGTTGCGAAAATTTAT |
| | | | | TTAAAGTTCGACTGGGATATTTCTAATCTACTAGCTACAAACACTTG |
| | | | | AGCTAAAATTGAACCTTGAACATGGTAAGTAAATCATTTTGGAGAG |
| | | | | ATTTCATGCTTTCTCTGTATCTTAATTCTATTTGTCTAATTGGTTTGGT |
| | | | | CGCAACCCGAAACATGGTTAGAATTACATAAAAAACACAACGAAGT |
| | | | | GAAATCCGGTAGCTCGACCTTCGTTCGTTGGGAGTTTTTCCTCGTGT |
| | | | | CTTGGGTCACTGTAATTGGCACGAGGTTTTTTCACTCCTTTTGTAGTT |
| | | | | CTTACAGTTAATATTTCAGAAAGAGTCTAAATTTATTGTATTGCTTTT |
| | | | | TTTAGATTGTAATTATAATTATACCCTATTACAACTAGTGCCTTGCTA |
| | | | | GTTGTTTATTTTAAATAATATTTTCTGTCGTTCTAGGAAAAAAAAAAT |
| | | | | TGAACCTTGAACACTTAATGCGGAAGTAGTTAATACTTGCAAACTTA |
| | | | | AGGAATAAGTTAAGTAGGCTTTGATGCTATAGATAGTCCTAAGCCA |
| | | | | TCACTCTTCAAAACACAATTTACACAATATTTCTAGAAAAATCAAAA |
| | | | | CTTCCATAGATATGTGAAAATAATAAATGGATGTATATACAAAAAA |
| | | | | AAAACATAATAAAAAATTGAAAAACTTATACACGCGTACAAGCCAA |
| | | | | TGATTATAGTCTAACTGGTTAGAGACATTTTAGGTTTATCTAAAGTT |
| | | | | TTGAGTCGATCTTTAGGAATGATAAGTTTAGGTTTTTTTTTTCTCTGA |
| | | | | ATATTTATAACGGTCCATGGTCAACATTTTGTTTTCTAATATACGTGC |
| | | | | AAGACTTACCATTATATGGTGATGTTTCTCTAATGTCACATACCGAC |
| | | | | CAAATGTTTGTACAAAAATAAAAATAAATACGTGTATAAAAGTCGG |
| | | | | CAAACGAAAATTAAAAAAACAGGATCTCATACATTCACTTCCACACA |
| | | | | CACTCCTCCTAGCTCTCATTTTTTATTTCCTTTTTCATCCCCAGCAAAA |
| | | | | ACACGCACACATAAATTAAATTCAAACGAATCAAACCCTAAAAACA |
| | | | | ACCAAAAAAAAATAATACAATCTCACACACACATTTTCAAACATGGC |
| | | | | GGCCGTAACTTCACCAAACCCACCTTTCACCACCACCACCACAAAAC |
| | | | | CACCGCATTCCGCCACCACATTTCACCGTCCCACCACCACTCATTTAT |
| | | | | CCTTCCCAACCCCACCAAAACGAAACCGTTTCCTTCACATCACCAAC |
| | | | | GTCCTCTCGGACCACAAACCCATCACTACCACCACTACAACCACCGT |
| | | | | TCCACCACCTTCATTCACTTCCCGTTTCGCCGCCGACCAACCCCGGA |
| | | | | AGGGCTCCGACGTTCTCGTAGAAGCCCTCGAACGTGAAGGCGTCAC |
| | | | | CGACGTCTTCGCTTACCCTGGTGGCGCGTCAATGGAGATCCACCAA |
| | | | | GCTCTCACGCGCTCCACCACCATCCGCAACGTCCTCCCCCGCCATGA |
| | | | | ACAAGGTGGCATCTTCGCCGCAGAAGGCTACGCACGTGCCTCCGGT |
| | | | | CTCCCCGGCGTCTGTATAGCCACTTCCGGTCCCGGCGCCACAAACCT |
| | | | | TGTCTCCGGCCTTGCTGACGCGCTTCTTGACAGTGTCCCCGTCGTTG |
| | | | | CCATCACCGGCCAAGTTCCCCGGCGAATGATCGGAACTGATGCTTTT |
| | | | | CAAGAAACACCCATTGTTGAGGTAACAAGATCCATTACTAAACATA |
| | | | | ACTATTTGGTTTTAGATGTTGATGATATTCCAAGAATTGTTAGAGAA |
| | | | | GCTTTTTATCTTGCCCGGTCGGGTCGACCCGGACCCGTTTTGATTGA |
| | | | | TATTCCAAAAGATATACAACAACAGTTATGTGTGCCTAAATGGGAT |
| | | | | GAGCCCATGAGGCTTCCTGGTTATTTATCTAGGTTACCTAAGCCACC |
| | | | | TAATGATGGTTTACTAGAACAAATTGTGAGGTTAGTTGGAGAGTCG |
| | | | | AAAAGGCCGGTTTTGTATGTAGGTGGTGGTTGTTTGAATTCGAGTG |
| | | | | ATGAGTTGAGACGGTTTGTTGAGCTTACGGGTATTCCGGTAGCTAG |
| | | | | TACTTTAATGGGTCTTGGGTCTTATCCAGCTTCGAGTGATTTGTCTCT |
| | | | | TCAAATGCTTGGAATGCATGGAACTGTTTATGCGAATTATGCTGTTG |
| | | | | ATAAGGCGGATTTGTTGCTTGCGTTTGGGGTTAGGTTTGATGACCG |
| | | | | TGTGACTGGAAAGCTTGAAGCTTTTGCTAGTAGAGCTAAGATTGTT |
| | | | | CATATTGATATTGACTCTGCTGAGATTGGGAAGAATAAGCAGCCAC |
| | | | | ATGTGTCGGTTTGTGGTGATATTAAGATTGCGTTACAGGGTCTTAAC |
| | | | | AAGATTTTAGAAGGGAGGCGTGAGATGAGTAATCTTGATTTTTCGG |
| | | | | CGTGGAGGGCAGAGTTGGATGAACAAAAGGTGAACCATCCGTTGA |
| | | | | GTTTTAAAACGTTCGGTGAAGCTATTCCACCGCAATATGCTATTCAG |
| | | | | GTGCTTGATGAGTTAACGGGTGGGAATGCTATTATAAGTACTGGGG |
| | | | | TCGGGCAACATCAGATGTGGGCTGCTCAGTTTTACAAGTACAACAG |
| | | | | ACCTAGACAGTGGTTGACTTCAGGTGGACTAGGGGCTATGGGCTTT |
| | | | | GGACTGCCTGCAGCCATTGGTGCAGCCGTTGCAAGACCTGATGCAG |
| | | | | TGGTAGTGGATATCGATGGTGATGGAAGTTTTATCATGAATGTTCA |
| | | | | AGAGTTGGCTACTATCCGAGTGGAAAATCTTCCTGTCAAGGTTTTGT |
| | | | | TACTCAATAATCAGCATTTGGGTATGGTGGTTCAATGGGAGGATCG |
| | | | | TTTCTACAAGGCAAATAGGGCGCATACCTACTTGGGGAACCCATCA |
| | | | | AAGGAGGCTGAAATATTTCCAAACATGTTGAAGTTTGCCGAAGCGT |
| | | | | GTGATATACCAGCTGCACGAGTGACCCGTAAAGCTGATCTACGGGC |
| | | | | GGCTATTCAGAAGATGTTGGATACTCCTGGGCCTTACTTGTTGGAT |
| | | | | GTCATTGTGCCGCATCAAGAACATGTTTTGCCGATGATTCCTGCTGG |
| | | | | TGGGGGCTTCATGGATGTGATCACCGAGGGTGATGGTAGAACGAA |
| | | | | GTATTGAGCTTCAAAGTCACAACTTAGTTTCAAGTTTCAACCACTGT |
| | | | | ATGTAAACATCTAGCATCTTTACTTAAGAGGTGTGAGCAGTTTGTTG |
| | | | | GATTTGAAGTTAATTTCTTGTTGTGTATTTTAGTTTCTGGTTTTTTGA |
| | | | | ATGTTCCGTAGTAGTGTATTGACTGTTCTTTTAGTAGCCTCATGAAT |
| | | | | AAATGTCTTTTTTTCCTTGCCTGCGTAGTTTTGTAGAAGTAATCTTCT |
| | | | | GTTTCTATGTTTATAGCAAGGACCAAAAGAAGAGTATTAGAGCCCC |
| | | | | TGGTCTAAAAGTCGGATATCTGAAGTCAAATTTTAGCCCCTATTAAG |
| | | | | ATATGTCTCTTTTTGATTGTGGTCAATACTAGCTGTAAGATACCATTT |
| | | | | GAGATCAGCAATTACCCGTTGCGGTTTCATTTTGGTTTGGTCTGGTC |
| | | | | TGGTCTGGTCTGGTCTATTTCTTGTTGTGTAGTTTCTGCTATTTGTGG |
| | | | | GCTATATGTTCTGGTTTTCTTGGGTTACTGTGTTTGGGCTTAGTATTA |
| | | | | CAATCTATTAGTTTTGAAGTTGGTTCATATTTAGTAAACTATAAATTT |
| | | | | TCAAAAACTATTCTTGATCGGTCCAGCATCAGTATATACTGGTTCAG |
| | | | | AAACTTGATAGATGTTGCTCAATCTTTCCGTCCTGAATGGGGCATTG |
| | | | | ATTGTTAGGATGTTTTTGTATAGGGTGACTAATATTTCTGGAAGGAT |
| | | | | GTTCGGTTTGGTAGTGTTTCCGAACAGGTGTAGGCTGCTTCTGAGT |
| | | | | GGGCAGAGTACGTAATCTCATCACCTACTCCTTGGGGAGGGTTTGA |
| | | | | CTTGATTTTGAGTCAAGGGTATAAGTCTTGCAATTGTAGTTTGCGTT |
| | | | | AGGCAGTGGTCAATGATTTATTACTAACTTAGCGTTATATTTTCCAG |
| | | | | CTACAAGTGCTATAAGTTAATGATTTAGTACTAACACAGAATGTAGT |
| | | | | TCATTTATGCATCAACGTAATTATTTTCCACACTAAATTTTTGTGTGT |
| | | | | GAGATGACAATACCGGATGATGAAACTTTTTATACTAAGCAAAGTG |
| | | | | AGTTTCTTGTTTGATCGAATGACTATGATTTATATATCACTTCCCAAA |
| | | | | TAGAACGCAATTAGGTGGTTTTAAGATTGGAGTAGAGCAGACCATC |
| | | | | TTCAAGAACTAGTCCAGAGCGAGTTGGATACTGGGAGTATACATCT |
| | | | | TTTGGGGTACAGTTTTGATGGCGTTTATTAATATTAATTTCAACAAA |
| | | | | CCATTTTACATGCTAAAAATAGTGGAGTACCTCAAGTTAAAACAGCT |
| | | | | CAAACAGGTTAAAATACATCTAAGTTTAAGGCACAAAACTGTCTAA |
| | | | | AAACTGTAAACATCTGTATTGCTCTAAAAAAAACTCCGATTTAAGGC |
| | | | | ACTTGTGGCACTGCTGTAGTTTCAAACGATTTCCTCCTAACAAGTAA |
| | | | | GCACGTAATATTTGAAGAACGAGTGCTCATGTTTTCAGGACTGCAT |
| | | | | AACACAAAGTCGGGCCTGTGGAGTGCTAAGTGGAGGCGATCTTCG |
| | | | | CCCATAACCATTGCAGTTGCATCAAGCAAAACATGCCATTGGTTTCT |
| | | | | ATGGGCTTCGGACACCCAATGCATTGAGTAGTGGGTCCCATTTACA |
| | | | | TTCGCAGGATAAGAGAACAAGCCTTTTGGGCTTTGCTTACTTTTCCT |
| | | | | TCTAAAATATTGACTGAGTTGTGAACCTTTGATCCTCAGATCTAACC |
| | | | | ATGTCTCGGGTGCTGATATCACTTTTGCGTCCTTGTAACCTGCAAAC |
| | | | | GCTTTTATGTAGTCATGTTCTTCGTTTATGATTGTCATGTAGTAATTA |
| | | | | CCCCTGAAAAATGGGTAACTTTCACCCACCAACATCATGGCATCACG |
| | | | | ATAGCTTGAGGTGAATAAAACAAGATACTCTTCATCAGGCAACCCA |
| | | | | CACCGCTTCAAAACTTTGTTTCTTGCTTGAATTTCAGGTATTGAGAT |
| | | | | GAAGCTTCCAAGATATGATGACTTTTTAGTAAGGATATCCAGTAATC |
| | | | | TTGATGGCTCGAGCTGAATTCTTACCAGATCATGGA |
| 33 | Euphorbia | cDNA | 2053 | CAATCCACCACCCCCCGCCGCTCTCTCCAAATCTCCAACTCCACTCCC |
| | heterophylla | Contig | | AAACCCACAATCCCCGCCCCCTCCGTCCCCTCCGCCCCCCAAACCCCT |
| | | | | CCCCCGCGGTTCGCCCCCGACGAGCCCCGCAAGGGCGCCGACATTC |
| | | | | TCGTCGAGGCCCTGGAGCGCCAGGGCGTCACCGACGTGTTCGCCTA |
| | | | | CCCCGGCGGCGCCTCAATGGAGATCCACCAAGCCCTAACCCGCTCC |
| | | | | CCAACCATCCGCAACGTCCTCCCCCGCCACGAGCAGGGCGGCGTCT |
| | | | | TCGCCGCTGAAGGATACGCCCGCGCCTCCGGCAAGCCCGGCGTGTG |
| | | | | CATCGCGACCTCCGGCCCAGGCGCCACCAATCTCGTCAGCGGCCTC |
| | | | | GCCGATGCGCTCCTCGACAGCGTCCCCATTGTGGCCATCACCGGCC |
| | | | | AGGTGCCTCGCCGGATGATCGGAACCGACGCCTTCCAGGAAACTCC |
| | | | | GATTGTTGAGGTAACTCGATCCATAACGAAGCACAATTATTTGGTAC |
| | | | | TTGATATTGAGGATATCCCTAGGATTGTGAGTGAGGCTTTTTTCTTG |
| | | | | GCGTCCTCTGGTCGTCCTGGTCCAGTTTTAATCGATGTGCCTAAGGA |
| | | | | TATACAGCAGCAATTAGCTGTTCCTAATTGGAATGTATCCATGAAAT |
| | | | | TGCCTGGTTATCTATCTAGGTTACCGAAAGACCCTAGCGAATTGCAA |
| | | | | TTAGAGCAGATTGTGAGGCTAATTTCCGAGTCTAAGAAACCAGTTTT |
| | | | | GTACGTTGGAGGTGGGTGTTTGAATTCCAGTGAGGAATTGAGGAA |
| | | | | ATTTGTTGAATTAACCGGGATTCCAGTTGCTAGTACTTTGATGGGTT |
| | | | | TAGGATCTTTCCCACTTAACCATGACTTATCCCTGTCAATGCTCGGA |
| | | | | ATGCATGGAACTGTTTATGCCAATTATGCAGTGGACAAAAGTGATC |
| | | | | TTTTACTTGCATTTGGAGTACGATTCGATGATCGTGTGACTGGAAAG |
| | | | | CTCGAAGCTTTTGCAAGCCGGGCTAAAATAGTTCACATCGACATCG |
| | | | | ATTCGGCGGAAATCGGGAAAAACAAGCAGCCCCATGTGTCTATATG |
| | | | | TGGAGATGTTAAATTAGCCTTGCAGGGAGTCAACAAAATTCTGGAG |
| | | | | AGCAAAAGCTTCAAGAGTAAGTTAGATTTCGGGAAATGGAGAGAC |
| | | | | GAGTTAAATGACCAAAAAGTTAAATATCCATTGAATTTCAAGACTTT |
| | | | | TGATGAAGCAATTCCTCCTCAGTATGCCATACAAGTTCTCGACGAAT |
| | | | | TAACCGATGGAAATGCCATAATAAGTACAGGAGTCGGACAACATCA |
| | | | | AATGTGGTCGGCCCAATTTTACAAGTACAAAAAGCCGAGGCAATGG |
| | | | | CTAACTTCCGGAGGGTTAGGTGCTATGGGTTTCGGACTTCCGGCAG |
| | | | | CAATAGGTGCTTCGGTTGCTAACCCTAATGCCGTTGTGGTTGATATC |
| | | | | GATGGGGATGGGAGTTTCATAATGAATGTCCAGGAGTTAGCCACG |
| | | | | ATCCGAGTCGAAAATCTACCAATCAAAATATTGCTTCTGAATAACCA |
| | | | | GCATTTGGGTATGGTTGTACAATGGGAAGACCGTTTTTACAAGGCG |
| | | | | AATCGAGCTCATACTTATTTGGGGGACCCATCGAAAGAGTCGGAGA |
| | | | | TTTTCCCCAATATGTTGAAGTTTGCTGAAGCTTGTGGAATTCCTGCT |
| | | | | GCTCGAGTGTCGAGAAAACAGGATATAAGAGGGGCAATTCAGACA |
| | | | | ATGTTGGATACTCCTGGTCCGTACCTTTTGGATGTGATTGTGCCACA |
| | | | | TCAAGAACATGTGTTGCCTATGATCCCAAGTGGCGGCGGGTTTAAG |
| | | | | GATATAATAACGGAGGGTGATGGGAGATTCAAGTACTAGTCTTAGT |
| | | | | CTTGTTGAGGTAATAAAATGTATGTTTTTTGTGTAAGTTGATGCTTT |
| | | | | GGTATTAGGTGTGGTTGGTCTTTTGGTTTATGGTTATGTGTTTAATG |
| | | | | TTGTTGTTGGTTTGTGTTTGGACTTGAAACGTGTTGCATTTGCTATG |
| | | | | TTCTTGTTAGCTTGGCAAATGATTAAAGTTATCTGTAATTGTTCTTGT |
| | | | | ACTTCTAGG |
| 34 | Euphorbia | gDNA | 4912 | TTCAGTTTAGTTCAATTCAGCAAGTTGCAGTTCAAAAGAACAGAGCC |
| | heterophylla | Contig | | TTAATGTCCAATATTGAATTGTTTGAAATGTACTATTACCATATGTTT |
| | | | | TAAAAAGTGTTGTCAGTCAGATAGTCGAGACTTCGAAATAATTACA |
| | | | | ATGGACTTGGACGGAATCTAGATGGGAGTTAAATAAATTTATATTTT |
| | | | | TTAAATATTATTTAATATATAAAAAATATAAATTTAATTAAAAATAAA |
| | | | | TTATAATAAAATAAAAAAAATAATTTAAATTTTTAAAATAACATTCTA |
| | | | | AAAAGTCCACTAAACGATATTATAAAAATTTCGACTTAAATACCTTT |
| | | | | TTGGGTTCTGTTCTATACACCAAATGCCCTTTTGGTGTCTGACCTATT |
| | | | | GAGATCGCCTTTTTTTATGTCTGACGTATTCAATATTTGCTTATTTGG |
| | | | | TACATTTCATATTCAATTTCAGTATGATAACTTAATCGACTAAAAAG |
| | | | | AGTCAACAATTTATGCATGATCAACAAAATATATTCATGTGTTCAAA |
| | | | | CCATATTATATGCATTTCATATTCTTTGTGTTGTTAGATGGATGCTCG |
| | | | | ATGTACTAAAATAATATAGATCAAACACTAAATAGGCTAATGTTGAA |
| | | | | TACATCAGGCACCAACTAGGCAAAAAGTGAATACATCACGCACCAA |
| | | | | ACAGACAATTTCAATAGGTCAAACACCAAAAAAGTATTTAATATATA |
| | | | | AAACAGGACCTAAAAAAATATTCAATTATACAAAATTTCCTACTATT |
| | | | | TAAGATCACAACAACATTCTAAAAAATTCTACGTAACCGCGAGCTAG |
| | | | | CTCGAGTTATAAAACACTTTGTGTTTAGTAAATAGTGAAGGTCATAA |
| | | | | AGGTAATTTGGGGTGGACCTTCAAATTTTAAAAGGGAATCGAGGGC |
| | | | | CACATATCACACTCCCTCTTCCGCCACAAAACCTGCAACTCTCTCCTT |
| | | | | CTACTCTCCACAATGGCGGCGGCGGCGCCTTCCTCTGCAGCCACCAC |
| | | | | CATCTCCAAACCCTCCGCCGTCAGATCCTCAATCTCCGCCTCCCGATT |
| | | | | CTCCCTCCCGTTCCCCATCAAACCCCAATCCACCACCCCCCGCCGCTC |
| | | | | TCTCCAAATCTCCAACTCCACTCCCAAACCCACAATCCCCGCTCCCTC |
| | | | | CGTCCCCTCCGCCCCCCAAACCCCTCCCCCGCGGTTCGCCCCCGACG |
| | | | | AGCCCCGCAAGGGCGCCGACATTCTCGTCGAGGCCCTGGAGCGCC |
| | | | | AGGGCGTCACCGACGTGTTCGCCTACCCCGGCGGCGCCTCAATGGA |
| | | | | GATCCACCAAGCCCTAACCCGCTCCCCAACCATCCGCAACGTCCTCC |
| | | | | CCCGCCACGAGCAGGGCGGCGTCTTCGCCGCTGAAGGATACGCCC |
| | | | | GCGCCTCCGGCAAGCCCGGCGTGTGCATCGCGACCTCCGGCCCAGG |
| | | | | CGCCACCAATCTCGTCAGCGGCCTCGCCGATGCGCTCCTCGACAGC |
| | | | | GTCCCCATTGTGGCCATCACCGGCCAGGTGCCTCGCCGGATGATCG |
| | | | | GAACCGACGCCTTCCAGGAAACTCCGATTGTTGAGGTAACTCGATC |
| | | | | CATAACGAAGCACAATTATTTGGTACTTGATATTGAGGATATCCCTA |
| | | | | GGATTGTGAGTGAGGCTTTTTTCTTGGCGTCCTCTGGTCGTCCTGGT |
| | | | | CCAGTTTTAATCGATGTGCCTAAGGATATACAGCAGCAATTAGCTGT |
| | | | | TCCTAATTGGAATGTATCCATGAAATTGCCTGGTTATCTATCTAGGT |
| | | | | TACCGAAAGACCCTAGCGAATTGCAATTAGAGCAGATTGTGAGGCT |
| | | | | AATTTCCGAGTCTAAGAAACCAGTTTTGTACGTTGGAGGTGGGTGT |
| | | | | TTGAATTCCAGTGAGGAATTGAGGAAATTTGTTGAATTAACCGGGA |
| | | | | TTCCAGTAGCTAGTACTTTGATGGGTTTAGGATCTTTTCCACTTAACC |
| | | | | ATGACTTATCCCTGTCAATGCTCGGAATGCATGGAACTGTTTATGCC |
| | | | | AATTATGCAGTGGACAAAAGTGATCTTTTACTTGCATTTGGAGTACG |
| | | | | ATTCGATGATCGTGTGACTGGAAAGCTCGAAGCTTTTGCAAGCCGG |
| | | | | GCTAAAATAGTTCACATCGACATCGATTCGGCGGAAATCGGGAAAA |
| | | | | ACAAGCAGCCCCATGTGTCTATATGTGGAGATGTTAAATTAGCCTTG |
| | | | | CAGGGAGTCAACAAAATTCTGGAGAGCAAAAGCTTCAAGAGTAAG |
| | | | | TTAGATTTCGGGAAATGGAGGGACGAGTTAAATGACCAAAAAGTT |
| | | | | AAATATCCATTGAATTTCAAGACTTTTGATGAAGCAATTCCTCCTCA |
| | | | | GTATGCCATACAAGTTCTCGACGAATTAACCGATGGAAATGCGATA |
| | | | | ATAAGTACAGGAGTCGGACAACATCAAATGTGGTCGGCCCAATTTT |
| | | | | ACAAGTACAAAAAGCCGAGGCAATGGCTAACTTCCGGAGGGTTAG |
| | | | | GTGCTATGGGTTTCGGACTTCCGGCAGCAATAGGTGCTTCGGTTGC |
| | | | | TAACCCTAATGCCGTTGTGGTTGATATCGATGGGGATGGGAGTTTC |
| | | | | ATAATGAATGTCCAGGAGTTAGCCACGATTCGAGTGGAAAATCTAC |
| | | | | CGATCAAAATATTGCTTCTGAATAACCAGCATTTGGGTATGGTTGTA |
| | | | | CAATGGGAAGACCGATTTTACAAGGCGAATCGAGCTCATACTTATT |
| | | | | TGGGGGACCCATCGAAAGAGTCGGAGATTTTCCCCAATATGTTGAA |
| | | | | GTTTGCTGAAGCTTGTGGAATTCCTGCTGCTCGAGTGTCGAGAAAA |
| | | | | CAGGATATAAGAGGGGCAATTCAGACAATGTTGGATACTCCTGGTC |
| | | | | CGTACCTTTTGGATGTGATTGTGCCACATCAAGAACATGTGTTGCCT |
| | | | | ATGATCCCAAGTGGCGGCGGGTTTAAGGATATAATAACCGAGGGT |
| | | | | GATGGGAGATTCAAGTACTAGTCTTAGTCTTGTTGAGGTAATAAAA |
| | | | | TGTATGTTTTTTGTGTAAGTTGATGCTTTGGTATTAGGTGTGGTTGG |
| | | | | TCTTTTGGTTAATGGTTATGTGTTTAATGTTGTTGTTGGTTTGTGTTT |
| | | | | GGACTTGAAACGTGTTGCATTTGCTATGTTCTTGTTAGCTTGGCAAA |
| | | | | TGATTAAAGTTATCTGTAATTGTTCTTGTACTTTTAGGTAATAATATG |
| | | | | CTTCCTAGATGAGGTTTTGAAGAGGTTTTGCTCTTGTTTTCTTTCACT |
| | | | | AATTTTTTGTTAGTGAAGTAGAAAATTCTCCAAAGAGGGCAAAGTA |
| | | | | TAAGAAAAAAACCAATATATAATATTGTAGATCTATAGTTGATTATA |
| | | | | GATGAAATAAGTTATCCTAAACTTCTAGGAGCTAAATAGAACTTATT |
| | | | | GGAGCAGGACTAAACTAAATATATGAGATATGAATTGAACTGACTG |
| | | | | AAATTTAAGAATAGAATTCCAAGAGGACAAGACCAAAACAGAAGA |
| | | | | TTGTAGTAATTGAGTCCATGAAAAAAGATTTGATTTCAAAAGGGCC |
| | | | | TCATCGTAAAATAATTGTTCATAATAGCAATTAGTCAATTATTCATCA |
| | | | | GTGGGTTCGTTTAAGATTCTTAATCTTAATTTCTCAAATGAAATCCTA |
| | | | | GAAAGTTTCCAACTTTACTAGGAAAAGAGCAATATTATTGACTAAA |
| | | | | AATGAATTAAATGTTTGGTCCACAAAAGTTTGGAATTTTCATGTTTTT |
| | | | | GCCAGATTGCCATCAAGTGGAAAACATGTCTGCAGTTGGATAAGAA |
| | | | | ATGGAAAATCTATGTGAAGTTGTGCAAAGGAAGGCCATACAATTAT |
| | | | | TTTGGCAGATAATGTAAAGCCAAAATACAATATGCACTGCTTTTAAA |
| | | | | AGTTTATGTGCAACAGTTTGAATAGTTAGTTCAGACTTTTAGCAGTA |
| | | | | TTATAAATGGTTAGGTCATATTTTCGGAGACTACAATTCAGCTCAGT |
| | | | | TTGGAGTAAAATTCAATTCAGTTGTATGTATTTATCGATTTACATTCA |
| | | | | AATAAGGCCTTGTTTAGTTTGGGTCATATTAATTTAGTTCAACCAAC |
| | | | | TTTAATTTTATTTACTTTAGTTTTAAAAAATATAAAATAACATGACTT |
| | | | | TTGTTATACAAATGTAACTTCAACTCAAAAACACAACAAAAGAGTTT |
| | | | | TCTATATCAATGTAATTTTGGGGTATTTATCTTGGCTTAAAAAGTTTC |
| | | | | TTATAATAACTTTTTAACTTATAAAAAGTAAAAGAAGTGTTTCTTTCA |
| | | | | GCTGATTTTTAATTTAAAAGTAAAAATATAGAAGTAAGGATTTGTTA |
| | | | | CTTTTCTACTTTTGCTTTTCTTTAAGACAAAAGCTATAAGCTAAGAGA |
| | | | | AACACCCCTTTATCAACATTGGAAGTTGTTTACGGGCTAGGTAGCAC |
| | | | | AAAATTGGAAACTGAAACGGAAATGAGAAAACCCATTTTCAAAACG |
| | | | | TATAGGTTTGGATACCGTAAGGAAACGTGTAAATAATAAAAAATGT |
| | | | | ATGGATATATTTATAAATATAAAATTTTTTATGCATATAATTTCATTT |
| | | | | TTTCATAAAATATTAAAATATTATTTAAAATATATTCATTAATTACTA |
| | | | | AAGACATCTCCAACCCTCGCTATCTCTTTTTCCCCTAAAACTTTTTACT |
| | | | | CTCCAACTCTACCTTTTAAATTTTTAGTTCAAAACACATATATTATTG |
| | | | | ATTTATTCTTACATATCTAACTTTTTATTATCATTTTATTCCTCCTTTCC |
| | | | | TATCTAACTCAAACAAGCATAAACTCAATTAAATAATAAATAAAAGA |
| | | | | AAAGGCAAAAAAAATTAAGGGATGAACAATGCTGTTTTAGAGCAAC |
| | | | | ATCCTCTCAAATTTGAGGCAAAGAATGAAGCTCCATTATTCATCTAC |
| | | | | AACCCTAAAATAGGGTTAGGTTGGAGATGGCGTAAACAATTTTATT |
| | | | | TCATGCTTTCCCTTGGTTGTATGGTTCGTCCGAGGGAGAAATCGTAA |
| | | | | ATTCATCAATATCAAGTTCTTAAAAAGAACACAATTTCAAC |
| 35 | Euphorbia | gDNA | 3185 | CGTCGAGGCCCTGGAGCGCCAGGGCGTCACCGACGTGTTCGCCTAC |
| | heterophylla | Contig | | CCCGGCGGCGCCTCAATGGAGATCCACCAAGCCCTAACCCGCTCCC |
| | | | | CAACCATCCGCAACGTCCTCCCCCGCCACGAGCAGGGCGGCGTCTT |
| | | | | CGCCGCTGAAGGATACGCCCGCGCCTCCGGCAAGCCCGGCGTGTG |
| | | | | CATCGCGACCTCCGGCCCAGGCGCCACCAATCTCGTCAGCGGCCTC |
| | | | | GCCGATGCGCTCCTCGACAGCGTCCCCATTGTGGCCATCACCGGCC |
| | | | | AGGTGCCTCGCCGGATGATCGGAACCGACGCCTTCCAGGAAACTCC |
| | | | | GATTGTTGAGGTAACTCGATCCATAACGAAGCACAATTATTTGGTAC |
| | | | | TTGATATTGAGGATATCCCTAGGATTGTGAGTGAGGCTTTTTTCTTG |
| | | | | GCGTCCTCTGGTCGTCCTGGTCCAGTTTTAATCGATGTGCCTAAGGA |
| | | | | TATACAGCAGCAATTAGCTGTTCCTAATTGGAATGTATCCATGAAAT |
| | | | | TGCCTGGTTATCTATCTAGGTTACCGAAAGACCCTAGCGAATTGCAA |
| | | | | TTAGAGCAGATTGTGAGGCTAATTTCCGAGTCTAAGAAACCAGTTTT |
| | | | | GTACGTTGGAGGTGGGTGTTTGAATTCCAGTGAGGAATTGAGGAA |
| | | | | ATTTGTTGAATTAACCGGGATTCCAGTTGCTAGTACTTTGATGGGTT |
| | | | | TAGGATCTTTCCCACTTAACCATGACTTATCCCTGTCAATGCTCGGA |
| | | | | ATGCATGGAACTGTTTATGCCAATTATGCAGTGGACAAAAGTGATC |
| | | | | TTTTACTTGCATTTGGAGTACGATTCGATGATCGTGTGACTGGAAAG |
| | | | | CTCGAAGCTTTTGCAAGCCGGGCTAAAATAGTTCACATCGACATCG |
| | | | | ATTCGGCGGAAATCGGGAAAAACAAGCAGCCCCATGTGTCTATATG |
| | | | | TGGAGATGTTAAATTAGCCTTGCAGGGAGTCAACAAAATTCTGGAG |
| | | | | AGCAAAAGCTTCAAGAGTAAGTTAGATTTCGGGAAATGGAGAGAC |
| | | | | GAGTTAAATGACCAAAAAGTTAAATATCCATTGAATTTCAAGACTTT |
| | | | | TGATGAAGCAATTCCTCCTCAGTATGCCATACAAGTTCTCGACGAAT |
| | | | | TAACCGATGGAAATGCCATAATAAGTACAGGAGTCGGACAACATCA |
| | | | | AATGTGGTCGGCCCAATTTTACAAGTACAAAAAGCCGAGGCAATGG |
| | | | | CTAACTTCCGGAGGGTTAGGTGCTATGGGTTTCGGACTTCCGGCAG |
| | | | | CAATAGGTGCTTCGGTTGCTAACCCTAATGCCGTTGTGGTTGATATC |
| | | | | GATGGGGATGGGAGTTTCATAATGAATGTCCAGGAGTTAGCCACG |
| | | | | ATCCGAGTCGAAAATCTACCAATCAAAATATTGCTTCTGAATAACCA |
| | | | | GCATTTGGGTATGGTTGTACAATGGGAAGACCGTTTTTACAAGGCG |
| | | | | AATCGAGCTCATACTTATTTGGGGGACCCATCGAAAGAGTCGGAGA |
| | | | | TTTTCCCCAATATGTTGAAGTTTGCTGAAGCTTGTGGAATTCCTGCT |
| | | | | GCTCGAGTGTCGAGAAAACAGGATATAAGAGGGGCAATTCAGACA |
| | | | | ATGTTGGATACTCCTGGTCCGTACCTTTTGGATGTGATTGTGCCACA |
| | | | | TCAAGAACATGTGTTGCCTATGATCCCAAGTGGCGGCGGGTTTAAG |
| | | | | GATATAATAACTGAGGGTGATGGGAGATTCAAGTACTAGTCTTAGT |
| | | | | CTTGTTGAGGTAATAAAATGTATGTTTTTTGTGTAAGTTGATGCTTT |
| | | | | GGTATTAGGTGTGGTTGGTCTTTTGGTTTATGGTTATGTGTTTAATG |
| | | | | TTGTTGTTGGTTTGTGTTTGGACTTGAAACGTGTTGCATTTGCTATG |
| | | | | TTCTTGTTAGCTTGGCAAATGATTAAAGTTATCTGTAATTGTTCTTGT |
| | | | | ACTTTTAGGTAATAATATGCTTCCTAGAAGAGGTTTTGCTCTTGTTTT |
| | | | | CTTTCACTAATTTTTTGTTAGTGAAGTAGAAAATTCTCCAAAGAGGG |
| | | | | CAAAGTATAAGAAAAAAACCAATATATAATATTGTAGATCTATAGTT |
| | | | | GATTATAGATGAAACAAGTTATCCTAAACTTCTAGGAGCTAAATAG |
| | | | | AACTTATTGGAGCGGGACTAAACTAAATATATTAGATATGAATTGA |
| | | | | ATTGAATTGAAATGACGAAATTTAAGAATAGAATTCCAAGAGGATG |
| | | | | AGGCCAAAACAAAAGATTGTAGTAATTGAGTCCATGAAAAAAGATT |
| | | | | TGATTTCAAAAGGGCCTCATAGTAAAATAATTGTTCATAATAGCAAT |
| | | | | TAGTCAATTATTCATCAGTGGGTTCGTTTAAGATTCTTAATCTTAATT |
| | | | | TCTCAAATGAAATCCTAGAAAGTTTCCAACTTTACTAGGAAAAGAGC |
| | | | | AATATTATTGACTAAAAATGAATTAAATGTTTGGTCCACAAAAGTTT |
| | | | | GGAATTTTCATGTTTTTGCCAGATTGCCATCAAGTGGAAAACATGTC |
| | | | | TGCAGTTGGATAAGAAATGGAAAATCTATGTGAAGTTGTTGTGCAA |
| | | | | AGGAAGGCCATACAATTATTTTGGCAGATAATGTAAAGCCAAAATA |
| | | | | CAATATGCACTGCTTTTAAAAGTTTATGTGCAACAGTTTGAATAGTT |
| | | | | AGTTCAGACTTTTAGCAGTATTATAAATGGTTAGGTCATATTTTCGG |
| | | | | AGACTACAATTCAGTTTAGTTGGGAGTACAATTCAGTTCAGTTGTGT |
| | | | | GTAGTTATCAATTTACATTCAAATATGGCCCTGTTTAGTTCAGGTCAT |
| | | | | TTTAGTTCATCTCAACTAACTTTAATTTAATTTACTTTAGCTTTAAAAA |
| | | | | ATTTAAAATAACATGACCTTTGGTATATAATTGTAACTTCTACTCAAA |
| | | | | AAAACAACAAAAGAGTTTTCTATATCAATGTAATTTCATCAACATCG |
| | | | | GAAGTTTTTTATGGGATAGGTAGCACAAAAATGAAAACTAAAACGA |
| | | | | AATTGGGAAAACCATTTTCAAAAAGTATAAGTTTGGAAATGTGTAA |
| | | | | ATAATAAAAAATATATATGGATATATTTATAAATATAAAATTTTTTAA |
| | | | | GCATATAATTTCATTTTTTGTATAAAATATTAAAGTATTATTTAAAAT |
| | | | | ATATTCATTACTCTCTCCGGTTCCAAATAAAAGACCTTTTGGCCTTTT |
| | | | | TTATTTGGTTCTAAATATTTGATCATTTAGATTATCCATGCAATATTT |
| | | | | ATTATTCTTTCTC |
| 36 | Commelina | cDNA | 363 | CTTTCGTTGAGGATGCTTGGAATGCATGGTACTGTGAGCGCAAATT |
| | diffusa | Contig | | ACTCAGTTGATAAGTCTGATTTGTTGCTTGCTTTTGGGGTTAGGTTT |
| | | | | GATGATAGGGTGACTGGGAAGCTGGAGACATTTGCTAGTAGGGCG |
| | | | | AAGATTGTGCACATTGATATTGATAGGGCTGAGATTGGTAAGAACA |
| | | | | AGCAGCCCCATGTGTCGATTTGTGCTGATATCAAGCTGGCTTTGCAG |
| | | | | GGGATGAATGCGATGTTGGAGGAGAGTGGTGTTTATAAGAAGTTT |
| | | | | GACTTTGGTGCGTGGAGGGAGGAGTTGGATGTGCAGAAGAAGACT |
| | | | | TATCCTTTGAGCTACAAGACATTTGGGGATTTGATTCCTCCG |
| 37 | Digitaria | cDNA | 1868 | CGTTGATAAACTGATCCATAACATTTTTAACCAGGAACGAAATTACA |
| | sanguinalis | Contig | | CAACAAAACATTAACTTCAAAACCGACAAACTGCTTACAGAACACAC |
| | | | | TATATGTGACATTGAAGCTCAATATTTCATTCTGCCGTCGCCTTCGG |
| | | | | TGATCACATCCATGAAACCTCCACCAGCCGGGATCATGGGCAACAC |
| | | | | ATGTTCTTGATGGGGCACGATCACATCCAACAAGTAAGGCCCCGGT |
| | | | | GTATCCAACATCTTCTGAATAGCTGCTCGTAGATCTGCCTTCCGGGT |
| | | | | CACTCGGGCAGCTGGGATATCACACGCTTCAGCAAACTTCAACATG |
| | | | | TTAGGGAATATTTCAGACTCTTTTGACGGATTTCCTAAGTAGGTATG |
| | | | | AGCCCGATTCGCCTTGTAAAACCGATCCTCCCACTGAACCACCATAC |
| | | | | CCAAATGCTGATTGTTAAGTAACAAAATCTTAACAGGAAGATTTTCA |
| | | | | ACACGGATTGTGGCTAACTCTTGAACGTTCATTATAAAGCTTCCATC |
| | | | | ACCATCGATATCAACTACTACCGCATCAGGTCTTGCAACAGCCGCCC |
| | | | | CGATAGCAGCGGGCAACCCAAAACCCATCGCCCCTAGTCCACCTGA |
| | | | | CGTCAGCCATTGTCTAGGCTTGTTGTATTTGTAAAACTGAGCAGCCC |
| | | | | ACATCTGATGCTGCCCGACCCCAGTGCTAATAATCGCATTCCCACCC |
| | | | | GTTAACTCATCAAGCACTTGAATGGCATACTGCGGAGGAATAGCTT |
| | | | | CGCCAAATGTTTTAAAACTCAACGGATACTTAACCTTTTGCTCATCCA |
| | | | | ATTCCTTCCTCCAGTTCGAGAAATCAAGATTAGTCACCGAATTCTTTA |
| | | | | CCTCCAAAATCTTGTTCAGACCCTGTAACGCGACCTTAATATCACCA |
| | | | | CAAATCGACACATGAGGCTGCTTATTCTTCCCAATTTCCGCAGAATC |
| | | | | AATATCAATATGAACAATCTTAGCTCTGCTAGCAAAAGCCTCAAGCT |
| | | | | TCCCCGTCACACGGTCATCAAACCTTACCCCAAACGCAAGCAACAAA |
| | | | | TCACTCTTATCAACCGCATAATTCGCATAAACCGTCCCATGCATCCCA |
| | | | | AGCATATGCAGCGACAAATCACTAGAAGCAGGGTATGCTCCAAGCC |
| | | | | CCATCAACGTACTCGCAACCGGTATCCCCGTAAGCTCCACAAACCAA |
| | | | | GAAAAGGACGCTGATGTTCTCGTCGAAGCTCTGGAACGTGAAGGC |
| | | | | GTTACAGACGTCTTCGCTTACCCAGGTGGCGCCTCCATGGAGATCC |
| | | | | ACCAAGCTCTCACGCGCTCACCACCATCCGCAACGTTCTCCCACGTC |
| | | | | ACGAACAGGGCGGCGTCTTTGCTGCCGAAGGCTACGCACGTGCCTC |
| | | | | CGGTCTTCCCGGCGTCTGTATTGCAACCTCTGGTCCTGGAGCTACGA |
| | | | | ACCTAGTAAGTGGTCTTGCTGATGCTTTATTAGACAGTGTTCCAATG |
| | | | | GTTGCTATTACTGGTCAAGTTCCCAGGAGAATGATTGGAACAGATG |
| | | | | CGTTTCAAGAAACCCCTATTGTTGAGGTAACACGTTCCATTACTAAG |
| | | | | CATAATTATTTAGTTTTGGATGTCGAGGATATTCCCAGGATTGTTAG |
| | | | | GGAAGCTTTTTATCTTGCGTCTTCTGGTCGACCCGGACCGGTTTTAA |
| | | | | TTGATGTACCTAAGGATATACAGCAGCAGTTGGTAGTGCCTAAATG |
| | | | | GGATGAGCCTATTAGGTTACCTGGGTATTTGTCTAGGTTGCCTAAAA |
| | | | | CGGAGAATAATGGGCAGTTGGAACAGATTGTTAGGTTGGTGAGTG |
| | | | | AGGCCAAGAGGCCGGTTTTGTATGTGGGGGGTGGGTGTTTGAATT |
| | | | | CGGGGGATGAGTTGAGGCGGTTTGTGGAGCTTACGGGGATACCGG |
| | | | | TTG |
| 38 | Digitaria | gDNA | 5667 | GGAGTAGGGACCACATACAACAGAGTATGATACTATACAATGCATC |
| | sanguinalis | Contig | | TAAACCATGATTCAAGAATATAATCATATGGTAATCATGCATTAGGA |
| | | | | TCCACTGACAGCAGCAGATCTTTGTATGGTTATATGTTCATATTCCG |
| | | | | ATGGACAATTTAACAAACAAAACAAATCCTTGTGGATCCATTGTGG |
| | | | | GGATATTCATTTTCTCAGTACAAGACACATTGAACAAATGGTTAGAC |
| | | | | AAACCCTTGTCTTCCCTTCCTCCTCTGGTTAAGAAGAAGTCTTCAATC |
| | | | | TCACTCAGAGCCCAAAGAACACAATCTATAAAAAATTGAAACGAAA |
| | | | | GGCCTAATAAGAGTATATCAATATACACTAGGTTACATGTGTTTGCA |
| | | | | ACCAGGGTCGTGAAAGCCAATGCTACTAAGTAGTTTTATCCATTACA |
| | | | | TGGAACTCAATATTCAAGAAAAAACATATCCATGATTGCAACTACAA |
| | | | | TTTTAACCATATGTGACTACTGAAAATCAAAACTTTTGCAGGAACAT |
| | | | | TGCAGCTATCCACAGCTCCACTATCCCCTGAATTGCTGGATTGTGAT |
| | | | | CCTTTATATTTTTTTTTCCAGAGCCCGCTAGAACCATAAACTGATACA |
| | | | | AATCAAACATACGAGTTAATTTTACTAGGATTGAAATTTTTATACGG |
| | | | | CAGATCATAAAACCATCCATCGCTTTGAAATGGTAATTTAATCAATC |
| | | | | ATGCCAACACGACAGTACAGGACATGCTTATATGCACAAACCATAT |
| | | | | ACGTGGCCTAAAACTCTTCAGGGCAGTTATTCAAAAACCAAACCAA |
| | | | | CAGAGGCACCTACTAGGGTTTTACATATCTGACAAGACGCGTCATG |
| | | | | ATCACTTGAGCCAGGCAAAATATCCCAAGGGGATCACAAATATCAT |
| | | | | ATTTTATTCTGGTTGTCTCACTCCGTCAAGCACAGGCTATCAAGGAA |
| | | | | CAATAAACAAAAACTGCCCTCATTGATCAAGACCGCCTAATAATATT |
| | | | | GCAAAGCTCAGACCTATCGCCAGCGAAACACAAGAAGGGTCTAAAT |
| | | | | TTATCCTGAAATTAAGAAAGAAATAAAATTAAACAAGCATGACAGT |
| | | | | TCCTCTCACTGTAGGGTTTGCAGCATTGCTTGAAGATGTGCCGAAA |
| | | | | GGGGAGGGAACAGAGCGGGGGGTGGGAGGTGACCTCTGTAGTCG |
| | | | | ACGCCGGAGTTGAGCTTGACGACGACGGGTCGGCCCCTGATGGAC |
| | | | | TTGAGAAAGTCCGACGGCGTCTTCACCGCCCCGCCGCCCGAGCCGG |
| | | | | GCTTGTCGCCGCCGCCGCTGCTCATCTTGCGCGCTATGCCCGTGTGT |
| | | | | TCGCGCATCGCGGGCGAGAGGCGACCCGTTTGGGCTCCGGATTGG |
| | | | | CGGATCCCTTCGTGCCGACCTCGTCCGGTCCACCCGTTTAGCTGGAC |
| | | | | CCCACGTGCCAGTGAGCATACAGGAAAACGACAGTCGACAACCAC |
| | | | | GTCCGCAGTCCAGTCCAAATCGTTTTTTACTTCACCATGTCGTTACCA |
| | | | | GCGCACCTTTCTAAAGTCACTGACACGTCGGTCCCACGTAGGCTTGC |
| | | | | CCCACCGGTCAGTGTGAAGCGTGTATACACGTTTGTGGAGGTGTCG |
| | | | | CCGTCGCTTTACGGCAGCCATCCATGATCCATCTCAACCACACATCT |
| | | | | CGCACGCAAATAAAGTAAGCCAGCCAGGCACGCCCTCCCCACTCTC |
| | | | | CCCATCTCCGTGCCACCCCCCAAAGTCTCAAACCCTCGCCGCCGCCT |
| | | | | CCGAGACAGCCGCCGCCACCATGGCCACGACGACCGCCGCCGCCAC |
| | | | | CGCCGCGGCCGCCGCGCTGACCAGCACCACCACCACCTCCGCGCGC |
| | | | | AGGCCGAGGAGCCGACCCGCGCCCCTCCCGTTCGCCCGTCGCGCCG |
| | | | | GGCCCATCCGGTGCTCCGCGGCGTCGCCGGTGACGGCCCCGGCCAC |
| | | | | CCCCGCCACACCGCTCCGGCCGTGGGGCCCCACCGAGCCCCGCAAG |
| | | | | GGCGCCGACATCCTCGTCGAGGCCCTCGAGCGCTGCGGCGTCCGCG |
| | | | | ATGTCTTCGCCTACCCCGGCGGCGCGTCCATGGAGATCCACCAGGC |
| | | | | GCTCACCCGCTCCCCCGTCATCGCCAACCACCTCTTCCGCCACGAGC |
| | | | | AAGGGGAGGCCTTCGCTGCCTCCGGGTACGCGCGCTCCTCCGGCCG |
| | | | | CGTCGGCGTCTGCGTCGCCACCTCGGGCCCCGGCGCCACCAACCTC |
| | | | | GTCTCCGCGCTCGCCGACGCGCTGCTCGATTCGGTGCCCATGGTCG |
| | | | | CCATCACGGGGCAGGTGCCCCGCCGCATGATCGGCACCGACGCTTT |
| | | | | CCAGGAGACGCCGATCGTCGAGGTTACCCGCTCAATCACCAAGCAC |
| | | | | AACTACCTGGTCCTCGACGTCGAAGACATCCCCCGCGTTGTGCAGG |
| | | | | AGGCGTTCTTCCTCGCCTCCTCTGGTCGCCCCGGGCCGGTGCTCGTG |
| | | | | GACATCCCCAAGGATATCCAGCAGCAGATGGCGGTGCCGGTCTGG |
| | | | | AACACGCCCATGAGTCTGCCAGGGTACATTGCGCGACTCCCCAAGC |
| | | | | CTCCTGCGACTGAATTGCTTGAGCAGGTGCTGCGTCTTGTTGGTGA |
| | | | | GTCACGCCGCCCTGTTCTTTATGTTGGTGGTGGCTGCGCGGCATCC |
| | | | | GGTGAGGAGTTGCGCCGCTTTGTGGAGATGACCGGAATCCCAGTG |
| | | | | ACAACTACTCTGATGGGCCTTGGCAACTTTCCCCAGTGACGACCCAC |
| | | | | TGTCTCTCCGGATGCTTGGTATGCATGGTACCGTGTATGCAAATTAT |
| | | | | GCGGTGGATAAGGCCGACCTGTTGCTTGCATTTGGTGTGCGGTTCG |
| | | | | ATGATCGTGTGACAGGGAAAATTGAGGCTTTTGCAAGCAGGGCTA |
| | | | | AGATTGTGCACATTGACATTGATCCAGCTGAGATTGGTAAGAACAA |
| | | | | GCAGCCACATGTGTCCATCTGTGCGGATGTCAAGCTTGCTTTGCAG |
| | | | | GGCATGAATGCTCTTCTGGAAGGAATCACATCAAAGAAGAGTTTCG |
| | | | | ACTTTGGCCCATGGCAAGATGAGTTGGATCAGCAGAAGAGGGAAT |
| | | | | TCCCCCTAGGGTACAAATCTTTCGATGAGGAGATCCAGCCACAATA |
| | | | | CGCTATCCAGGTTCTGGATGAGCTGACAAAAGGGGAGGCCATCATT |
| | | | | GCTACCGGTGTTGGGCAGCACCAGATGTGGGCAGCACAGTACTAC |
| | | | | ACTTACAAGCGGCCACGGCAATGGTTGTCTTCAGCTGGTCTTGGTG |
| | | | | CTATGGGATTTGGTTTGCCGGCTGCGGCTGGTGCTGCTGTGGCCAA |
| | | | | CCCAGGTGTAACAGTTGTTGACATCGATGGGGATGGTAGCTTCCAA |
| | | | | ATGAACATTCAGGAGTTGGCTTTGATTCATATTGAGAACCTTCCAGT |
| | | | | GAAGGTCTTTGTGCTAAACAACCAGCACCTGGGAATGGTGGTGCAG |
| | | | | TGGGAGGACAGGTTCTACAAAGCCAACCGAGCACACACATACTTGG |
| | | | | GGAACCCAGATAATGAGAGTGAGATATATCCGGATTTTGTGACGAT |
| | | | | TGCCAAAGGATTCAACATCCCAGCAGCCCGTGTGACAAAGAAGAGC |
| | | | | GACGTCCGTGCAGCAATCAAGAAGATGCTTGAGACTCCAGGTCCCT |
| | | | | ACCTGTTGGATATCATCGTCCCACACCAGGAGCATGTGTTACCTATG |
| | | | | ATCCCGAGTGGTGGCGCTTTCAAGGATATGATACTGGATGGTGATG |
| | | | | GCAGGACCGTGTATTGATCTGAACTTCAGCGAGTGCTGTTCTTGCCT |
| | | | | TTCTTTGACATGCATATGAGCTAGTAAAAGAGTGATGCGTGTCTTAC |
| | | | | CTATGTAATGTTCTCCTTTGTTTCTTCGATTCCTAGGGCGTCAACTCT |
| | | | | GGACTGCGTCATTTTCTAATGTGCTTGTCTGATGTACTCTGGTGTGG |
| | | | | TAATCTTAGCTTCCAACCTTCTAGTCCTGCAGTCTGTTGTTATCGTTA |
| | | | | GTGCAGGCATATATGCATCATAAGAGATCATGTAAGTGCCTTTTGCT |
| | | | | ACATAATAAATAAGTTAATAAATGCTGCTATATATGCAGTGGGTTCT |
| | | | | GAATTCTGTTCAGTTGCCAACTAGTTGTTGCTTTTGTTCTCGATTTCT |
| | | | | TTCCCTTGTTTCTACGATTCTCCTTTAGCTGATTCCAATTCAGCATAG |
| | | | | GAGTATGTTGTATGAAAGCATTGAGCAATTGTGCATGAAAGGATAG |
| | | | | GGTGTACAGCGGTGCAGCGCACAAGGCACTCACCGAAAATCAGCTT |
| | | | | GAGCTGCACAAAGGAACCATCTCCAAACATCTGTGCATGAAACACT |
| | | | | GTTGCATTTCTCATTGCGATGTACGCGGGCACGCTGGTCAAAGAAA |
| | | | | GTTCAGCGCCTTCCAAGACACCGGAGCAGTGCAGGGTTGTGAACAA |
| | | | | AGATCACAGCTGCGGCCTTTCGCGGATGGTGAAGAAGATATGCTG |
| | | | | GAGCAACGATCAGTATGGTTCCCACGGAAGAAGAAAAGGTAAAGC |
| | | | | TGTTACAGATGCTGGGCCACACAAGTAGACTTGTCAATCTGGCCCA |
| | | | | ATAAGACTTTGGGCCTCCAAGATTTGGGCCCGTATTTTTGGACCAAG |
| | | | | ACAAGGCCTCATTACGAATTTCGAGGTGTTAAAAAAATCACGAATTT |
| | | | | CTATGCTAGAAGATGTTTTAGGTGTGGGTGCTTCTTTGATACCATCA |
| | | | | TTAAGAACTAAAAATCCTCTCCTACGCGTATGAGTGTTTAAATAGGC |
| | | | | CCAACGCCCTTGTTACATAGTACGCTGAGCACCTGAAGCAAACTTTG |
| | | | | GACATCCTTTCCTTTGTAAAAGCAGCCATCCCATAAGACATGGCAAA |
| | | | | AACTAGACCTTTCAGTACTTGTACTTCTCTATTAAGGCCCTAGAATCA |
| | | | | AGTTTCAGATGAGGAAGCTCAATTAAGAATGTGTTTAGATTGGGAG |
| | | | | AGAAAACACTGTAGATCGTGGTAGGTGTAAGTGGATGAATAGTAA |
| | | | | AAAGTTTGATGAATAATAGAAAATTAATGATAAACAGTAAAAAGTG |
| | | | | CGTTGGAGCCTAAGCTTATTGGGACCTCACTGGTCAATCCACGAGTT |
| | | | | TCTATCTCGTGAATCGTGATCCATTTTGTGCCATTGATGAGAGACGG |
| | | | | CAAAGGGCGCGCCTCACTATACCAAAAAAAAAAAAAAACTCCCTTC |
| | | | | TGTTGGACCGGACAAAAGAGAGAAAGAATCGATGAGCGCGATATA |
| | | | | TGGTGACTGGATCATAATTGTTGCTGGGCTATGCTCACCGAACGGG |
| | | | | CCCGTACAGAGGCCCTTGCCATTAACTGCTAGGATCTCGACTTTTTC |
| | | | | TTTATCAGATTCGTTCGTCCATGATCGGACGGACAAATGCACAGCA |
| | | | | AAACGTCCATTCAGAGCGTGGCGGAGATTACATCATTGCACTATGT |
| | | | | ACACACCACAGTGTTGATCACGCATTTCTCCTGCAAAGACGACGAA |
| | | | | ATTGTAGGCACACGGCGTCACGTAACAACGACGTGAAGCCCAGCTA |
| | | | | CCGTGCGACCGTACGTTCGTGCACGTGTCGGCTAAATAGGGAGCAA |
| | | | | CGTGTTGCCAAAACATTTTCAAGAGCCCTCAAGGTTTGAGCACGGC |
| | | | | TACTTTTTCTTTTCTTTTCTTTTTTTGCCGAAAATGAACTTCATGGATC |
| | | | | TTAGCAGATATTTAAATTGAACATATGTCGTAGCTATGTATTTGGCA |
| | | | | CAAGAAAAATGTTGTGGCCGTTTGAATTTAGTAAATGTAGAGAGCC |
| | | | | TCCCCATGCATACCTGATCGGGTGGGCTCAAACCAATGCAGATTTGT |
| | | | | TGCAATCATAA |
| 39 | Kochia | cDNA | 2001 | ATGGCGTCTACTTCTGCAAATCCCACTTTTACCCCTTTCACCAGTAAA |
| | scoparia | Contig | | CCCCTTAAACCCCGTTCTCCCTTTCACTCTTTCCCATTTCCCTCAAACC |
| | | | | CCAAAACCCCTTCCTCTTCATTTCGCAACCTCAAAATCACATCTTCTC |
| | | | | TCTCTTCTTCACAACCCCCGAAACCACCTTCCGCCGTCAAAACCCACT |
| | | | | CACCACCTTCCCCTCTCACAACCGACGAACCCCCGCAAGGTTTTGTT |
| | | | | TCCCGATTTGCCCCTGACCAACCCAGAAAAGGCTGCGATGTCCTCGT |
| | | | | TGAGGCCCTCGAGCGGGAGGGCGTCACCGACGTGTTCGCTTATCCT |
| | | | | GGTGGCGCATCAATGGAGATTCATCAAGCTCTGACTCGCTCTGATTC |
| | | | | CATACGCAACGTCCTGCCTCGCCACGAGCAAGGCGGGATCTTTGCC |
| | | | | GCGGAGGGGTATGCTCGTGCCACGGGCCGTGTTGGTGTCTGCATT |
| | | | | GCGACATCTGGCCCTGGCGCTACGAACCTCGTGTCCGGGTTTGCTG |
| | | | | ATGCTTTGCTCGATTCCGTTCCACTGGTGGCGATCACGGGGCAGGT |
| | | | | GCCGCGGCGAATGATTGGGACGGATGCTTTTCAGGAGACTCCTATT |
| | | | | GTTGAGGTAACACGGTCTATTACCAAGCATAATTATCTGGTATTAGA |
| | | | | TGTTGAGGATATTCCTAGAATTGTTAAGGAGGCTTTCTTTTTGGCTA |
| | | | | ATTCTGGTAGACCTGGACCTGTTTTGATTGATATTCCTAAGGATATT |
| | | | | CAGCAGCAATTGGTTGTGCCTGATTGGGATCAGGGGGTTAGGTTAG |
| | | | | GTGGGTATGTGTCTAGGTTGCCGAAATCGGTGTTTTCGGCCAATGA |
| | | | | TGAGGGGCTTCTTGAGCAGATTGTGAGGTTGATGAGTGAGGCTAA |
| | | | | GAAGCCTGTGTTGTATGTGGGAGGCGGGTGTTTGAATTCTGGGGA |
| | | | | GGAGTTGAGGAAATTCGTCGAGTTGACTGGGATTCCGGTGGCTAGT |
| | | | | ACTTTAATGGGTTTGGGCGCTTATCCCTGTAATGATGACTTGTCTCT |
| | | | | TCATATGTTGGGTATGCACGGGACCGTGTATGCTAATTATGCTGTTG |
| | | | | ATAAGGCAGATTTGTTGCTTGCCTTTGGGGTTAGGTTTGATGATCGT |
| | | | | GTGACAGGGAAGCTTGAGGCGTTTGCTAGCCGGGCTAAGATCGTG |
| | | | | CATATTGATATTGATTCTGCTGAGATTGGGAAGAATAAGCAACCCC |
| | | | | ATGTGTCAATATGTGCTGATGTCAAGTATGCGTTGAAGGGTATGAA |
| | | | | TAAGATTTTGGAGTCTAGGAAAGGGAAGTTGAATTTGAATTACTCT |
| | | | | AGCTGGAGGGAGGAATTGGGTGAGCAAAAGAAGAAATTCCCATTG |
| | | | | TCTTTTAAGACCTTCGGGGAAGCGATTCCTCCTCAGTATGCCATTCA |
| | | | | GATGCTTGATGAGCTGACCAATGGTAACGCTATTATTAGTACTGGT |
| | | | | GTTGGGCAACATCAAATGTGGGCTGCTCAGCATTACAAGTACAGAA |
| | | | | ACCCTCGCCAATGGCTGACCTCAGGTGGGTTGGGTGCCATGGGTTT |
| | | | | TGGTCTACCAGCCGCCATTGGAGCTGCTGTGGCTCGACCTGATGCA |
| | | | | GTGGTGGTTGATATTGATGGCGATGGGAGTTTCATTATGAATGTTC |
| | | | | AAGAGTTGGCTACTATTAGGGTGGAAAATCTCCCTGTTAAGATAAT |
| | | | | GCTTTTGAATAACCAACATTTAGGTATGGTGGTTCAATGGGAAGAT |
| | | | | AGGTTTTATAAAGCCAATAGGGCACATACTTACCTTGGAAACCCTTC |
| | | | | AAAAGAGTCTGAAATCTTCCCGGATATGCTTAAATTTGCTGAGGCG |
| | | | | TGTGATATTCCTGCTGCTCGTGTCACCAAGGTTGGAGATTTGAGGG |
| | | | | CGGCCATGCAGACAATGTTGGATACTCCGGGACCTTACCTGCTTGA |
| | | | | TGTGATTGTACCTCATCAGGAGCATGTGCTGCCTATGATTCCTAGTG |
| | | | | GTGCAGCCTTCAAGGATATCATTAACGAAGGTGATGGAAGAACAA |
| | | | | GTTATTGA |
| 40 | Kochia | cDNA | 2001 | ATGGCGTCTACTTCTGCAAATCCCACTTTTACCCCTTTCACCAGTAAA |
| | scoparia | Contig | | CCCCTTAAACCCCGTTCTCCCTTTCACTCTTTCCCATTTCCCTCAAACC |
| | | | | CCAAAACCCCTTCCTCTTCATTTCGCAACCTCAAAATCACATCTTCTC |
| | | | | TCTCTTCTTCACAACCCCCGAAACCACCTTCCGCCGTCAAAATCCACT |
| | | | | CACCACCTTCCCCTCTCACAACCGACGAACCCCCGCAAGGTTTTGTT |
| | | | | TCCCGATTTGCCCCTGACCAACCCAGAAAAGGCTGCGATGTCCTCGT |
| | | | | TGAGGCCCTCGAGCGGGAGGGCGTCACCGACGTGTTCGCTTATCCT |
| | | | | GGTGGCGCATCAATGGAGATTCATCAAGCTCTGACTCGCTCTGATTC |
| | | | | CATACGCAACGTCCTGCCTCGCCACGAGCAAGGCGGGATCTTTGCC |
| | | | | GCGGAGGGGTATGCTCGTGCCACGGGCCGTGTTGGTGTCTGCATT |
| | | | | GCGACATCTGGCCCTGGCGCTACGAACCTCGTGTCCGGGTTTGCTG |
| | | | | ATGCTTTGCTCGATTCCGTTCCACTGGTGGCGATCACGGGGCAGGT |
| | | | | GCCGCGGCGAATGATTGGGACGGATGCTTTTCAGGAGACTCCTATT |
| | | | | GTTGAGGTAACACGGTCTATTACCAAGCATAATTATCTGGTATTAGA |
| | | | | TGTTGAGGATATTCCTAGAATTGTTAAGGAGGCTTTCTTTTTGGCTA |
| | | | | ATTCTGGTAGACCTGGACCTGTTTTGATTGATATTCCTAAGGATATT |
| | | | | CAGCAGCAATTGGTTGTGCCTGATTGGGGTCAGGGGGTTAGGTTA |
| | | | | GGTGGGTATGTGTCTAGGTTGCCGAAATCGGAGTTTTCGGCCAATG |
| | | | | ATGAGGGGCTTCTTGAGCAGATTGTGAGGTTGATGAGTGAGGCTA |
| | | | | AGAAGCCTGTGTTGTATGTGGGAGGCGGGTGTTTGAATTCTGGGG |
| | | | | AGGAGTTGAGGAAATTCGTCGAGTTGACTGGGATTCCGGTGGCTA |
| | | | | GTACTTTAATGGGTTTGGGCGCTTATCCCTGTAATGATGACTTGTCT |
| | | | | CTTCATATGTTGGGTATGCATGGGACCGTGTATGCTAATTATGCTGT |
| | | | | TGATAAGGCAGATTTGTTGCTTGCCTTTGGGGTTAGGTTTGATGATC |
| | | | | GTGTGACAGGGAAGCTTGAGGCGTTTGCTAGCCGGGCTAAGATCG |
| | | | | TGCATATTGATATTGATTCTGCTGAGATTGGGAAGAATAAGCAACC |
| | | | | CCATGTGTCAATATGTGCTGATGTCAAGTATGCGTTGAAGGGTATG |
| | | | | AATAAGATTTTGGAGTCTAGGAAAGGGAAGTTGAATTTGAATTACT |
| | | | | CTAGCTGGAGGGAGGAATTGGGTGAGCAAAAGAAGAAATTCCCAT |
| | | | | TGTCTTTTAAGACCTTCGGGGAAGCGATTCCTCCTCAGTATGCCATT |
| | | | | CAGATGCTTGATGAGCTGACCAATGGTAACGCTATTATTAGTACTG |
| | | | | GTGTTGGGCAACATCAAATGTGGGCTGCTCAGCATTACAAGTACAG |
| | | | | AAACCCTCGCCAATGGCTGACCTCAGGTGGGTTGGGTGCCATGGGT |
| | | | | TTTGGTCTACCAGCCGCCATTGGAGCTGCTGTGGCTCGACCTGATG |
| | | | | CAGTGGTGGTTGATATTGATGGCGATGGGAGTTTCATTATGAATGT |
| | | | | TCAAGAGTTGGCTACTATTAGGGTGGAAAATCTCCCTGTTAAGATA |
| | | | | ATGCTTTTGAATAACCAACATTTAGGTATGGTGGTTCAATGGGAAG |
| | | | | ATAGGTTTTATAAAGCCAATAGGGCACATACTTACCTTGGAAACCCT |
| | | | | TCAAAAGAGTCTGAAATCTTCCCGGATATGCTTAAATTTGCTGAGGC |
| | | | | GTGTGATATTCCTGCTGCTCGTGTCACCAAGGTTGGAGATTTGAGG |
| | | | | GCGGCCATGCAGACAATGTTGGATACTCCGGGACCTTACCTGCTTG |
| | | | | ATGTGATTGTACCTCATCAGGAGCATGTGCTGCCTATGATTCCTAGT |
| | | | | GGTGCAGCCTTCAAGGATATCATTAACGAAGGTGATGGAAGAACA |
| | | | | AGTTATTGA |
| 41 | Kochia | gDNA | 6022 | ATAGAAGTTATGGCAATCGCGGGGTGTTTGTTTTGTGGGAATAGAT |
| | scoparia | Contig | | GGGTGGGAATGGGAATTTTAATTCCAATGTCCTAACTCCCACGTTTG |
| | | | | TTTGTAGGGAATGAGATTATTTGAGAGAAGGGGAATGAGATTATTT |
| | | | | GGGGTCAATTTCAATAGTTTTATTACATGAGAGGTTGGTAACTTGGT |
| | | | | ATTTGGGTGGGAATTCATATTTCTTTTGTTTTTTTCTTTTGTTTTTGCT |
| | | | | GAAAGTTATTTCTTCATCTCATTTTATTTCCTTTTTTAAAAAAAAAATT |
| | | | | CTCATTTATTTCCAAATTAATACCAAGCAAGAGATTGAGATAATTTT |
| | | | | ATCAATTTTCACATTTCAATTTACATTCTCATTTTCATTTCCACTTGTC |
| | | | | AAATGTCCCGGCATGACATGGAACACGCAACACACCATAAGCTCAT |
| | | | | TAGATCATTTGTCGGAGTTTCGATTTTCAGCATATACACCTATTGACC |
| | | | | TGTCACCATCCATGGTAAAATGTTGATATGTGGGCTAAGCCACATCA |
| | | | | CGTAATGTCGTAATCTGCTTTTAAAAGCACATTAAGTTACATAATTTT |
| | | | | CAACACGCGCATTGCTATCAACTTGCATTGTTCCACTTGTATATCATT |
| | | | | GCTGAAGTATTATAATCGAGCGATCTGTTTTTGCACAATACAGAATC |
| | | | | GGATTTGCAACAGAAAATAGAAACAAGTAAAATCAGGATCAGATG |
| | | | | CTCAAAGCCTCATGTCATTGTTTCCATTTAAACAAAAAAACATGTCA |
| | | | | TTATAATTCCCATGCCCATATATTTAGGTAAATATTCCTGCTCGGATA |
| | | | | TTCTACGAAAAATAGAAAATTAAAGGCCATATAGGTCTAATGAACT |
| | | | | AGCTAAATCGAATCTGTAGAAGATTTTTAACATTTTAACTTTGTAAA |
| | | | | ATATGAAAATAATTATTCTTCTAAGCTTTGCACAATAGTCGCATTTTG |
| | | | | TTGTTGTCGTATAAAAAAAACATTGCATAGACAATAACTTAGATTTC |
| | | | | CAACGAATTAACTACTCCGTAACTTGTATTCCACGTGCATAGAAAAT |
| | | | | GTAGCATTTTATGTTTATTTCTCTATTTATAAGTGGGGCAAATCTTGT |
| | | | | AAATAACTATCTATGTATTCAATAAAAATAATCATGTGGTGTCTTGT |
| | | | | TTGATTTGTATTAATGAGTAGATTATCAACATTTAATTATTATAATTT |
| | | | | TTACATATCTACATTTAAAGATATAAACATTATAAGACATGTATTGA |
| | | | | GAAACATATAAATAGTAAATGACTGCGTTTTCACGGAACAAAATGA |
| | | | | ATACCTTATATAAAATGTAATTATTTGTTCATTTATTTTTAATTTTGTC |
| | | | | ACTGTATGACATTTTTAGTTATTTTTTTATTTTATTATTATATTTTATTT |
| | | | | TATACTTTCACCGTTTTTAAAATTTTTAAAAAAGTAATTTTTTATCTCT |
| | | | | CACAAAAAATGTCCCTTGTCGCAAGTTTTTGAAAACAAAAGAAGTA |
| | | | | ATAAAACTCAATTATGGATTGAGAGCTACATATAACATATATAAGAA |
| | | | | AATGAATGTATGACTCAACATACTCAATAAATAAATTGGATTTTGCG |
| | | | | ATCAAGTTTGTGGGCTTTATCCTTTCAATTAAAATGTATACTTGATGC |
| | | | | AACACAATCTAATGAGTAATGACATCTTTGATATGAAGTGTATCAAA |
| | | | | TATCTAAATAAATTATTTCAAAAATGGAGAACAAATATTAACCACGA |
| | | | | AATGAGTAGTAGAAAAAAGGAAACACCTTTTTTATTTGCATTATTAT |
| | | | | TATTACTTTATGTAGGAAAACTCCATTTGGAGTCTTGACAAAAACAA |
| | | | | ATGAATGAAAAAAGACTTTTTGGTTTTGGTAATTTAAGCTAAAAAG |
| | | | | AAAAAGAAAAAAGAAAAAAGAAAAAGGAGAAAACCAGAGTTGAG |
| | | | | CGCCTCCACTAGTTGTTTTCCTTTCCTTCCTCGGAAGAACCCTATTTC |
| | | | | TTCCTCCCTCATATGATATCCTTCCTTCAAACTCCACACTCCTCTCTTT |
| | | | | CATTTTCTCTCTGATCATACCTTCAACCTTCAACAATGGCGTCTACTT |
| | | | | CTGCAAATCCCACTTTTACCCCTTTCACCAGTAAACCCCTTAAACCCC |
| | | | | GTTCTCCCTTTCACTCTTTCCCATTTCCCTCAAACCCCAAAACCCCTTC |
| | | | | CTCTTCATTTCGCAACCTCAAAATCACATCTTCTCTCTCTTCTTCACAA |
| | | | | CCCCCGAAACCACCTTCCGCCGTCAAAACCCACTCACCACCTTCCCCT |
| | | | | CTCACAACCGACGAACCCCCGCAAGGTTTTGTTTCCCGATTTGCCCC |
| | | | | TGACCAACCCAGAAAAGGCTGCGATGTCCTCGTTGAGGCCCTCGAG |
| | | | | CGGGAGGGCGTCACCGACGTGTTCGCTTATCCTGGTGGCGCATCAA |
| | | | | TGGAGATTCATCAAGCTCTGACTCGCTCTGATTCCATACGCAACGTC |
| | | | | CTGCCTCGCCACGAGCAAGGCGGGATCTTTGCCGCGGAGGGGTAT |
| | | | | GCTCGTGCCACGGGCCGTGTTGGTGTCTGCATTGCGACATCTGGCC |
| | | | | CTGGCGCTACGAACCTCGTGTCCGGGTTTGCTGATGCTTTGCTCGAT |
| | | | | TCCGTTCCACTGGTGGCGATCACGGGGCAGGTGCCGCGGCGAATG |
| | | | | ATTGGGACGGATGCTTTTCAGGAGACTCCTATTGTTGAGGTAACAC |
| | | | | GGTCTATTACCAAGCATAATTATCTGGTATTAGATGTTGAGGATATT |
| | | | | CCTAGAATTGTTAAGGAGGCTTTCTTTTTGGCTAATTCTGGTAGACC |
| | | | | TGGACCTGTTTTGATTGATATTCCTAAGGATATTCAGCAGCAATTGG |
| | | | | TTGTGCCTGATTGGGATCAGGGGGTTAGGTTAGGTGGGTATGTGTC |
| | | | | TAGGTTGCCGAAATCGGTGTTTTCGGCCAATGATGAGGGGCTTCTT |
| | | | | GAGCAGATTGTGAGGTTGATGAGTGAGGCTAAGAAGCCTGTGTTG |
| | | | | TATGTGGGAGGCGGGTGTTTGAATTCTGGGGAGGAGTTGAGGAAA |
| | | | | TTCGTCGAGTTGACTGGGATTCCGGTGGCTAGTACTTTAATGGGTTT |
| | | | | GGGCGCTTATCCCTGTAATGATGACTTGTCTCTTCATATGTTGGGTA |
| | | | | TGCACGGGACCGTGTATGCTAATTATGCTGTTGATAAGGCAGATTT |
| | | | | GTTGCTTGCCTTTGGGGTTAGGTTTGATGATCGTGTGACAGGGAAG |
| | | | | CTTGAGGCGTTTGCTAGCCGGGCTAAGATCGTGCATATTGATATTG |
| | | | | ATTCTGCTGAGATTGGGAAGAATAAGCAACCCCATGTGTCAATATG |
| | | | | TGCTGATGTCAAGTATGCGTTGAAGGGTATGAATAAGATTTTGGAG |
| | | | | TCTAGGAAAGGGAAGTTGAATTTGAATTACTCTAGCTGGAGGGAG |
| | | | | GAATTGGGTGAGCAAAAGAAGAAATTCCCATTGTCTTTTAAGACCT |
| | | | | TCGGGGAAGCGATTCCTCCTCAGTATGCCATTCAGATGCTTGATGA |
| | | | | GCTGACCAATGGTAACGCTATTATTAGTACTGGTGTTGGGCAACAT |
| | | | | CAAATGTGGGCTGCTCAGCATTACAAGTACAGAAACCCTCGCCAAT |
| | | | | GGCTGACCTCAGGTGGGTTGGGTGCCATGGGTTTTGGTCTACCAGC |
| | | | | CGCCATTGGAGCTGCTGTGGCTCGACCTGATGCAGTGGTGGTTGAT |
| | | | | ATTGATGGCGATGGGAGTTTCATTATGAATGTTCAAGAGTTGGCTA |
| | | | | CTATTAGGGTGGAAAATCTCCCTGTTAAGATAATGCTTTTGAATAAC |
| | | | | CAACATTTAGGTATGGTGGTTCAATGGGAAGATAGGTTTTATAAAG |
| | | | | CCAATAGGGCACATACTTACCTTGGAAACCCTTCAAAAGAGTCTGA |
| | | | | AATCTTCCCGGATATGCTTAAATTTGCTGAGGCGTGTGATATTCCTG |
| | | | | CTGCTCGTGTCACCAAGGTTGGAGATTTGAGGGCGGCCATGCAGAC |
| | | | | AATGTTGGATACTCCGGGACCTTACCTGCTTGATGTGATTGTACCTC |
| | | | | ATCAGGAGCATGTGCTGCCTATGATTCCTAGTGGTGCAGCCTTCAA |
| | | | | GGATATCATTAACGAAGGTGATGGAAGAACAAGTTATTGATGTTCG |
| | | | | TATCGATGGTTGAAAGCATCTATAGAGGGGGAAGCAAAATAAGAA |
| | | | | TAATAATCTGTATGTATAATAGTATGTTCCTTTTAAATTTTTAGCGTC |
| | | | | TGTTTACTTGTTTTTTTAGTTTTCTAGTTAGTTTGTCGTTGTTATGTTG |
| | | | | CTTGTTACTTTGAGAATGCTTTTTTGTAGTTTTCAAGAGACGAGTAT |
| | | | | GGATGATCTTCCTATATTGTTCAAAGATTTCACCAAGTGGTCTTTGG |
| | | | | ACTATGTATGTTGTATTTGATGTTTGGTTAGTTATGTTAATTGTCCTA |
| | | | | ATGCAAGCGTTGTTAAAATCGCCATTTGACACTCATTAGACCTTACT |
| | | | | TCTATATATTTGTAAGCTGCTTATTGGATGATTTTAAACACCTCCTTG |
| | | | | TTTTGCTTGTCTAATGGACTTTTGTTGCTATACATAATAATGTTTAAC |
| | | | | TCCAAAACAACCAAAAAAAGATTCTGGCACAACCAGTTCTACTACCA |
| | | | | TAAAATAAGGTTATGAAGTGCTTTAGAGATAAGCACAAGCTTGGGA |
| | | | | AGATAGTAGTGGCTGCTCATACTTTTGGTTCTTCTGAAAAGAACATA |
| | | | | TGATTGTGAAGAAACTGCCTAAACATTTGCATTTCCTTGAGGTTGAG |
| | | | | GTTTGTAGATGATTAGTGTGAAAGCCCTTTGCAGGTGTTGAGAACT |
| | | | | TGAGATTGAGGTTTAGGTTAGAGATTTGGAGTGGAAGTTGGTAAGT |
| | | | | ACGACTTTTGCCGAAACTTCAGGAGCATAACGTGTCTCGGCAAGTT |
| | | | | AAGCAGGCAAGCATTTACAAGGAAAGGGTATTTGAACAATAGGTTT |
| | | | | ATATTGATCCCAAGAGTATGCTATACATTTGTCATGATCGAACAACT |
| | | | | TGCTACTTAAGAGATTTGTGTCATGCCTTTGAGTTATTGAGAAAAGG |
| | | | | AGTGAGCTAGATCCAGAATAACTTGAGGGAATTTAGAGACTTCATT |
| | | | | CCTATGAATCCAAATGGCCCGTAGTATGGTTAAAAACTGGCTACATT |
| | | | | TAATTCAATCTCTTTTAACAAATTAAAGGACAAAAATCTTGCAACCA |
| | | | | TTGTTCCAGTGAAATATTAGCTGCTAGGTCCGTGCGAATTCCTAGAA |
| | | | | CACCAAGATCGAAAAGTTCATAAACCCTAAATCCTGGCTTCCTTAAA |
| | | | | TTAGTTTTAACTGCTGAAGCATGGCATAGAACTTCATCTTCTTGTAA |
| | | | | ATACTTCCTCGGTTTGTTTTAGATGTTATATTTATTTGGGCCTAAACT |
| | | | | TTGGACCAAAATTAGAAAATTTTAATTAGAAGATGTAATTAGGGAA |
| | | | | ATGATGAATTAATTAAACAACTAACCAACCTAATCTCTACCACTAAA |
| | | | | CAACCTACCAACCACAGACCACCTCCAAACCCTAGCCGCCATCACCG |
| | | | | CCGCCGTCACCAAACCACCATCGACTACCAGACTCAAATCTCCAAAT |
| | | | | AGTCGCGACCACCACCAAACCCACGCCATGACCATCGACCACCACC |
| | | | | AAACCCTTGCTGCAAACCACACTTTGAGTCAACCACCGCAACAATTT |
| | | | | TTGAGCAATTTATGAGATTTTGAACTAATTTCGAACAACTTATGAGA |
| | | | | TTTTCAAAGAATTTCTCAGATTTCGAACTAATTGCTTTAAGATTTTGA |
| | | | | ACAATTTGACAAATTTCAAACCAATTTTTGAGATTTCAAATCAATTTC |
| | | | | CTCTAGCTTTCGAACCAACTTGAGGAGATTTCGAATAAGTTTTAAGA |
| | | | | ATTCGAATCAATTTCTTTGAGCTTTGACCGATTTGTGTGTTTTGGGT |
| | | | | GGTTCAATGGGGGCACTTTGGTGGTCTGTGTGGTGCTGTAAAACAT |
| | | | | TGATGTGCGAATGTGCGATTGGGGTGGTGCGATTGGGGTGGTGCG |
| | | | | GTGGTGTGTAGCTGGGTGGTATGCAGATATGGTGAGCCGTGGATG |
| | | | | TCATGGGTGGTTGTTGATGCGGTGTATTAGAGATATTAATTGCTTAA |
| | | | | GATGTTAATAATATTTGATTAGTTTATGTTTAATTGTGGGTACGACT |
| | | | | GTAAATCCACATGTAAAAAGTTACAA |
| 42 | Kochia | gDNA | 2514 | AAAATGGAGAACAAATATTAACCACGAAATGAGTAGTAGAAAAAA |
| | scoparia | Contig | | GGAAACACCTTTTTTATTTGCATTATTATTATTACTTTATGTAGGAAA |
| | | | | ACTCCATTTGGAGTCTTGACAAAAACAAATGAATGAAAAAAGACTT |
| | | | | TTTGGTTTTGGTAATTTAAGCTAAAAAGAAAAAGAAAAAGAAAAAG |
| | | | | GAGAAAACCAGAGTTGAGCGCCTCCACTAGTTGTTTTCCTTTCCTTC |
| | | | | CTCGGAAGAACCCTATTTCTTCCTCCCTCATATGATATCCTTCCTTCA |
| | | | | AACTCCACACTCCTCTCTTTCATTTTCTCTCTGATCATACCTTCAACCT |
| | | | | TCAACAATGGCGTCTACTTCTGCAAATCCCACTTTTACCCCTTTCACC |
| | | | | AGTAAACCCCTTAAACCCCGTTCTCCCTTTCACTCTTTCCCATTTCCCT |
| | | | | CAAACCCCAAAACCCCTTCCTCTTCATTTCGCAACCTCAAAATCACAT |
| | | | | CTTCTCTCTCTTCTTCACAACCCCCGAAACCACCTTCCGCCGTCAAAA |
| | | | | TCCACTCACCACCTTCCCCTCTCACAACCGACGAACCCCCGCAAGGT |
| | | | | TTTGTTTCCCGATTTGCCCCTGACCAACCCAGAAAAGGCTGCGATGT |
| | | | | CCTCGTTGAGGCCCTCGAGCGGGAGGGCGTCACCGACGTGTTCGCT |
| | | | | TATCCTGGTGGCGCATCAATGGAGATTCATCAAGCTCTGACTCGCTC |
| | | | | TGATTCCATACGCAACGTCCTGCCTCGCCACGAGCAAGGCGGGATC |
| | | | | TTTGCCGCGGAGGGGTATGCTCGTGCCACGGGCCGTGTTGGTGTCT |
| | | | | GCATTGCGACATCTGGCCCTGGCGCTACGAACCTCGTGTCCGGGTT |
| | | | | TGCTGATGCTTTGCTCGATTCCGTTCCACTGGTGGCGATCACGGGG |
| | | | | CAGGTGCCGCGGCGAATGATTGGGACGGATGCTTTTCAGGAGACT |
| | | | | CCTATTGTTGAGGTAACACGGTCTATTACCAAGCATAATTATCTGGT |
| | | | | ATTAGATGTTGAGGATATTCCTAGAATTGTTAAGGAGGCTTTCTTTT |
| | | | | TGGCTAATTCTGGTAGACCTGGACCTGTTTTGATTGATATTCCTAAG |
| | | | | GATATTCAGCAGCAATTGGTTGTGCCTGATTGGGGTCAGGGGGTTA |
| | | | | GGTTAGGTGGGTATGTGTCTAGGTTGCCGAAATCGGAGTTTTCGGC |
| | | | | CAATGATGAGGGGCTTCTTGAGCAGATTGTGAGGTTGATGAGTGA |
| | | | | GGCTAAGAAGCCTGTGTTGTATGTGGGAGGCGGGTGTTTGAATTCT |
| | | | | GGGGAGGAGTTGAGGAAATTCGTCGAGTTGACTGGGATTCCGGTG |
| | | | | GCTAGTACTTTAATGGGTTTGGGCGCTTATCCCTGTAATGATGACTT |
| | | | | GTCTCTTCATATGTTGGGTATGCATGGGACCGTGTATGCTAATTATG |
| | | | | CTGTTGATAAGGCAGATTTGTTGCTTGCCTTTGGGGTTAGGTTTGAT |
| | | | | GATCGTGTGACAGGGAAGCTTGAGGCGTTTGCTAGCCGGGCTAAG |
| | | | | ATCGTGCATATTGATATTGATTCTGCTGAGATTGGGAAGAATAAGC |
| | | | | AACCCCATGTGTCAATATGTGCTGATGTCAAGTATGCGTTGAAGGG |
| | | | | TATGAATAAGATTTTGGAGTCTAGGAAAGGGAAGTTGAATTTGAAT |
| | | | | TACTCTAGCTGGAGGGAGGAATTGGGTGAGCAAAAGAAGAAATTC |
| | | | | CCATTGTCTTTTAAGACCTTCGGGGAAGCGATTCCTCCTCAGTATGC |
| | | | | CATTCAGATGCTTGATGAGCTGACCAATGGTAACGCTATTATTAGTA |
| | | | | CTGGTGTTGGGCAACATCAAATGTGGGCTGCTCAGCATTACAAGTA |
| | | | | CAGAAACCCTCGCCAATGGCTGACCTCAGGTGGGTTGGGTGCCATG |
| | | | | GGTTTTGGTCTACCAGCCGCCATTGGAGCTGCTGTGGCTCGACCTG |
| | | | | ATGCAGTGGTGGTTGATATTGATGGCGATGGGAGTTTCATTATGAA |
| | | | | TGTTCAAGAGTTGGCTACTATTAGGGTGGAAAATCTCCCTGTTAAG |
| | | | | ATAATGCTTTTGAATAACCAACATTTAGGTATGGTGGTTCAATGGGA |
| | | | | AGATAGGTTTTATAAAGCCAATAGGGCACATACTTACCTTGGAAAC |
| | | | | CCTTCAAAAGAGTCTGAAATCTTCCCGGATATGCTTAAATTTGCTGA |
| | | | | GGCGTGTGATATTCCTGCTGCTCGTGTCACCAAGGTTGGAGATTTG |
| | | | | AGGGCGGCCATGCAGACAATGTTGGATACTCCGGGACCTTACCTGC |
| | | | | TTGATGTGATTGTACCTCATCAGGAGCATGTGCTGCCTATGATTCCT |
| | | | | AGTGGTGCAGCCTTCAAGGATATCATTAACGAAGGTGATGGAAGA |
| | | | | ACAAGTTATTGATGTTCGTATCGATGGTTGAAAGCATCTATAGAGG |
| | | | | GGGAAGCAAAATAAGAATAATAATCTGTATGTATAATAGTATGTTC |
| | | | | CTTTTAAATTTTTAGCGTCTGTTTACTTGTTTTTTTAGTTTTCTAGTTA |
| | | | | GTTTGTCGTTGTTATGTTGCTTGTTACTTTGAGAATGCTTTTTTGTAGT |
| 43 | Lolium | cDNA | 2152 | GCCGCCGCCGCCGCCATGGCAACTGCCACTTCGACAGCCGTCGCCT |
| | multiflorum | Contig | | TCTCGGGAGCCACCGCCACCTTGCCCAAACCACGCACTCTCCCGCGT |
| | | | | CACCAACTGCCCTCCTCACGCCGCGCCCTCGCCGCCCCCATCAGGTG |
| | | | | CTCCGCGGTGTCCCCATCGCCCTCGCCCGCCCCTCCCGCCACCGCGC |
| | | | | TCCGCCCATGGGGCCCATCCGAGCCCCGCAAGGGCGCCGACATCCT |
| | | | | CGTCGAGGCCCTCGAGCGCTGCGGCATCAGCGACGTCTTCGCCTAC |
| | | | | CCGGGCGGCGCCTCAATGGAGATCCACCAGGCGCTCACGCGCTCGC |
| | | | | CGCTCATCACCAACCACCTCTTCCGCCACGAGCAGGGCGAGGCCTT |
| | | | | CGCGGCGTCCGGGTACGCGCGGGCGTCCGGCCGCGTCGGGGTCTG |
| | | | | CGTCGCCACCTCCGGCCCGGGGGCCACCAACCTCGTCTCCGCGCTC |
| | | | | GCCGACGCCCTCCTCGACTCCATCCCCATGGTGGCCATCACGGGGC |
| | | | | AGGTCCCGCGCCGCATGATCGGCACGGACGCCTTCCAGGAGACGC |
| | | | | CCATCGTCGAGGTCACCCGCTCCATCACCAAGCACAACTACCTCGTC |
| | | | | CTCGACGTCGAGGACATCCCCCGCGTCATCCAGGAAGCCTTCTTCCT |
| | | | | CGCCTCCTCTGGCCGCCCGGGCCCGGTGCTCGTCGACATCCCCAAG |
| | | | | GACATCCAGCAGCAGATGGCTGTGCCCGTCTGGGACGCGCCCATGA |
| | | | | GTCTGCCAGGCTACATTGCCCGCCTGCCTAAGCCGCCGGCTACTGA |
| | | | | ATTGCTCGAGCAGGTCCTGCGTCTGGTTGGCGAGGCGAGACGCCC |
| | | | | GATTCTCTATGTTGGCGGTGGCTGCTCTGCGTCCGGGGAGGAGCTG |
| | | | | CGCCGCTTTGTTGAGCTCACTGGGATCCCTGTTACAACTACCCTCAT |
| | | | | GGGTCTTGGCAACTTCCCCAGCGACGACCCGCTGTCTCTGCGTATGC |
| | | | | TTGGGATGCATGGCACTGTGTACGCAAACTACGCCGTAGATAAGGC |
| | | | | TGACCTGTTGCTTGCATTTGGCGTGAGGTTTGATGATCGCGTGACT |
| | | | | GGGAAAATCGAGGCTTTTGCAAGCAGGTCCAAGATTGTGCACATTG |
| | | | | ACATTGATCCAGCTGAGATTGGCAAGAACAAGCAGCCGCATGTCTC |
| | | | | CATTTGCGCAGATGTCAAGCTCGCTTTGCAGGGCCTGAATGCTCTGC |
| | | | | TAACTGGGAGCAAAGCACACAAGAGTTTCGATTTTGCTTCGTGGCA |
| | | | | TGACGAGTTGGAGCAGCAGAAAAGGGAGTTTCCTCTGGGATACAA |
| | | | | AACTTTCGGGGAGGCCATCCCACCGCAATATGCTATCCAGGTACTG |
| | | | | GATGAGCTCACCAAAGGTGAGGCCATCATTGCCACTGGTGTTGGGC |
| | | | | AGCACCAGATGTGGGCGGCTCAGTATTACACCTACAAGCGCCCACG |
| | | | | GCAGTGGCTGTCTTCGGCTGGTCTGGGGGCAATGGGGTTTGGGTT |
| | | | | GCCAGCTGCAGCTGGCGCCGCTGTGGCTAACCCAGGTGTCACAGTT |
| | | | | GTTGACATTGATGGGGATGGTAGCTTCCTCATGAACATTCAGGAGT |
| | | | | TGGCGCTGATTCGCATTGAGAACCTCCCAGTTAAGGTGATGATATT |
| | | | | GAACAACCAGCATCTTGGAATGGTGGTGCAGTGGGAGGACAGGTT |
| | | | | TTACAAGGCCAATCGGGCGCATACATACCTTGGGAACCCAGAAAAT |
| | | | | GAGAGTGAGATATATCCAGATTTTGTGACCATTGCTAAAGGCTTCA |
| | | | | ATGTTCCTGCAGTTCGTGTGACAAAGAGGAGTGAAGTCCGTGCAGC |
| | | | | GATCAAGAAGATGCTTGAGACTCCTGGGCCATACTTGTTGGATATC |
| | | | | ATCGTCCCTCACCAGGAGCATGTGCTGCCTATGATCCCTAGCGGTG |
| | | | | GTGCTTTCAAGGACATTATCATGGAAGGTGATGGCAGGATTGCGTA |
| | | | | TTAATCGCAACTTCTGCAAGAGCTCCACCTACAAGACCTACAAGTGC |
| | | | | AATATGCCTGATCAGCATGATGCTGGTGTATGTTATATCCATGTGTT |
| | | | | CGCTAATTTGCTTGTTTGATGAGCTTGGTTTGCTAGTCTTACCTAGCT |
| | | | | CTGAACCTTCTAGGTTTTCTAGTCTGTTCTTTTCCGTAGGCATATGCT |
| | | | | GTCATAAGAGATCATGCAAGTTTCTTGT |
| 44 | Lolium | gDNA | 14102 | GGCCCAACACTGTCTACAGGAATAGAAGCGTGCGTAGACATCACTA |
| | multiflorum | Contig | | GGATTTATTGAATACATTCATGCTATATTGTTGCTGCCTAGTCTTTCG |
| | | | | TTCATTGCATATTTATTCAGATCAATAATTTTGTGGAAATATTAATCC |
| | | | | TCTTCATTATGAATCAAGATTTGATATCAATTTCTGCTCATGCTTACT |
| | | | | TTATTTCCAAGTGGGAAAAATTCACGTAGTTGTAGGTCACTAGATGT |
| | | | | TTTTGGGTATCCATTTGGATGAGCTGCATGTGGATCTATCAGAAGCA |
| | | | | TCGCTAGGTTGGTTTTGGTGTCTGTCTGAGAGGAGGTTTGTCTCACC |
| | | | | TTCTCCTAAACTTGATAACTTGTTTGCTGCCTGTGTGGATTGGAATTT |
| | | | | TATAGAAATTTTGGTTCCGGGAAATGTCAATCACATTGTTCTCTGAC |
| | | | | TGTTTATGTATGGTTTGGGTTGCTGATGTCTGCTTCCCTTCTGGAAC |
| | | | | AGGCTTTGGTTGGACCTCGGATCGAGCTTATGTTAGACTTTGACCCA |
| | | | | AGATATGATGACAAACTTATATGTGTTGTTGCTTCAAATGTCCTTCA |
| | | | | TTTGTTATTAGTGGATTAGGGAAAAACTTTCTGGTAGCAAACCAAGT |
| | | | | TGGCTATAGTGTTTATTGATTTGTTAGCTTGTTCCATGCCTGTCTGTA |
| | | | | TCCCCTTCCTCGTTTGTGGCGCATGAGTATTTGCTGCTGTCTGGAGG |
| | | | | CAATTATTATATCAAGTATGCAAGGTACTTAATTAATTTAGTACTACT |
| | | | | CTAGGTTTGTATCAAACCTGACACAACGTGTATTAGTTCAACAATGT |
| | | | | GTATATGCTTTCCTAGGGTCCTTTTACTGATTACTACAACAGTGTGT |
| | | | | GCTTGCTGTCTAGATGCCAGGCATAGGAAGTCAATGGTTGCTTTAC |
| | | | | ACCATCTTGGAGTACTAATTTTTCAACTAGACTGAATTTTCTCGCAGT |
| | | | | AGAGTAACTTCCTGATGGCAATGGTAAGTGGTTGCATATCAGAGAT |
| | | | | ACGCTGCTGCTAGAGTAGGCGAGGTGTGCTTCTTGGAACTGGCTAG |
| | | | | GTGCGAGCTCGTGAGATGCTGTTGAGCCTAAAGCAATGATGCCTTC |
| | | | | TCTGGATTAGGTAAGTCTTTGGTCTATCTCATTCTCCTCTTTGTTATTT |
| | | | | TGAGTCAAGTACCTATGTGATATGGAATATATGGTATGAATTGCTCT |
| | | | | GTGTTAGGGTCTAATCAAGTCACTATGATGTTACTTTATGGTTAATT |
| | | | | ATGTATGCGTTGCTTTCTTTTCGAGTTATGATGTAAGTGATAAGTCA |
| | | | | TAGTCATAATGATCTCCATAAAATGGAAATATTATACTCCAGTTATC |
| | | | | TATTTTCGAATTTATTGGTTGCATGATGTTCCAGGGGTAGCATCTAC |
| | | | | ATGACTAGCACTCTTTTATTTGACTTTAGGCCATGCTCTCATTTCTCT |
| | | | | TGCAGTAGATAGAGGCACAAATCTATTACAGTCTGATGCATATTTTT |
| | | | | CTTTGGAAGCATGAATGTTTTGTGAATCGTGAAACTGTTATTTTGGC |
| | | | | CAGTCCCTGATTCTTGAATGGTTTTTATTATTGGCTTGACCAGTTAA |
| | | | | GTATGTACTTGTGCACTAATTTTCCTCTGCATTGTTGTAGTCATGCAA |
| | | | | GATAGATGGATGAGCATACGGCGGAGTCTTGTTCTTGGACCGGTG |
| | | | | GTGGTTGGTAGCTGGATGTTGTGACGCGTTGCTAGAGGGAGCGGT |
| | | | | GCACACGGGGACAAGTTGGTGGGTTGCCATAGTGTTGGTGGACAG |
| | | | | GAGAGCTCCTGATCATGCAACTACAAGAAGGGGACTTGTAGGCATA |
| | | | | TATGCCTGGAGGTTGGGAGCTGTTTCCAGCCAGAAGGACCGATAAT |
| | | | | GGATGTTGCTGCTGTAGGGTGGGTTTACATTCTGGGCTAGCTTTATT |
| | | | | TGCATCCCTGTGTCGTTCTTGTATTTGTGGTTTGAACCGTGGATTACT |
| | | | | TATGTCTCCTTCAAAGAATTTCATGTCTTTGTTGTTTCCGATTGAATT |
| | | | | ATATTTTGTTTAATTTTGTCAAACTGTTAGAAAATAAAAACTTACAAA |
| | | | | TGGGAAGAAAAAATAGATCGCAAAAAGACTAAGGCCCTAAAAAAT |
| | | | | GTATAGGTTGTTAATTGATTAAGGCCCGAAAGGTTAATCAGCTAAC |
| | | | | AAAGATGAAGCCCAGAAGATAAATGGACCGAACAATGGCTGCAAA |
| | | | | AATACAAAGGCTCAAAATTTAAATGGATCGGAAAAGACCAAGGCCT |
| | | | | AGAATAAAAAGTCTGGTATTAATGGGCTCGGCCTATGCAATCCACC |
| | | | | GAAATGGATCGGGCTGATTTTGGATTACGACGATTAGATTTCGTCA |
| | | | | TAATTTTGCCATGTTAGACTAGCCACGTAGGATCTGATGTGGCATG |
| | | | | GCCAAATCTCCAGTGACGATATTGGATCGTCATAGTGGTTACGACG |
| | | | | ATCCAATATCATGATAGACGACTATGACGATTCAAAGTAAAAGGTC |
| | | | | ATGTTGGTTGATTTTTGACGCTCCGTTTTTGACGATCCATTTTTTGTC |
| | | | | ATAGAATCGTCATAGATGAAATATTATGACGATTTAGAGACGAAAA |
| | | | | TGGAGTGTCGTAGGTCAACATATTTCTTGTAGTGGAGAGAGTCCTC |
| | | | | AGTTAGTTCTATCTAACTTGGAGAATTCCGATGACGTCTTCACCAGC |
| | | | | CCGACACCACCAAAGCCGGCCTTCTTGCCACCACTCATCTTTCTTTCG |
| | | | | GTTTGTGTCTTGTTCGTGTGCGGGTCTCGCGCTAGGATCCGACTCCA |
| | | | | AGTAAGAGCATCTCCAACAGGCGCACTAAATTTTGGCGCGCTAAAC |
| | | | | GTCGCTTCCGCCACGCTGTAAACATATAGCACGCGCTGCGCTGAAA |
| | | | | TTTGCCCCACCGGATGCTCTATTTTGCAGCGCGTGTTGGCGCGGTAA |
| | | | | AAAAGCTCCTCCGTCGGGTGCGCCAAAAATACAACGCGCGCGGGC |
| | | | | GCGGCAAAATACAGCGCGCGCGCAGCAAACAACTTTCTACACTACT |
| | | | | ATGCATTCAACAAGATCAAACACTCACATATAAATCATGAAACAAAT |
| | | | | GATCTACTATAGTTCAAATGGACACAAAGTGCAACACACATGACAT |
| | | | | AGTTCAAGAACGACACACAACATGACGTAGTTCAACAACGACACAC |
| | | | | AACATATGGTTCAAATGGACACAAAGTGCAACACACAATTTGCATA |
| | | | | TCACAAATGCATCTCACACTTCCGCATTTTCCAAGTCATCACCTTATT |
| | | | | TTGCTTCTTCTTCCTCGTCATCTTGGGTTGAAGGAGGAATAGTAGGA |
| | | | | TCCATGGAGGCCACGAATCCTCCCGGTGGCGCTCCCGTAGTCCCAT |
| | | | | ACACACCACGCACCGAGCCTCCCATAGTCCCTTTGAAGACTCCTCCA |
| | | | | TAACTTGGTTCTCCCATGCCGGCCATGCCTCCAAAGCCTCCCATGCC |
| | | | | GGCCATGCCTCCCATGCCGGCCATGCCTCCCATACTTCCTCCAAATA |
| | | | | TGGGCATGCCTCCTCCAAACATGCCGGTCGTGGGTGTGTTCATGTT |
| | | | | GGCTACCAACAATCTCTTCTTGGCCAACACTTCGTCGCGAGCAAGGT |
| | | | | TGATGTACTACTTTTGCCTCTCATCCATGTGGGAAGTATCCATCAAG |
| | | | | AAAAGCTTCCTCTCCTCCTCCACTAGGCGTAGATGCTCCTCGTTGGC |
| | | | | TTGCCTCTTCTCCTCCAAATCCAACTTCCTCTCCTCGTTGGCGGCAAG |
| | | | | CCTCTCCTCCAAAGCGGCTCTCCGAGCTTCCATTGCATCCATAGCCTC |
| | | | | TTTCTCCAAGTTCCTTGCCACCTTCCTATCTTCATTTGCTTGCAACCTT |
| | | | | GCATTTGCAATCCTTTCCATAGCAATGACAATGTCATCATCTCCGGC |
| | | | | CTTCTTTCCCTTCATATCTTTGGCGGTCTTCCTACCAAGCACATTCCTC |
| | | | | CGCCCATTATTGACCGAGTGACGAGTGGAGCGTCTCTTCTTGCCTTG |
| | | | | GTCACTTGAATCATCATCATCGATGATTGTTGCATCACCGGTTGCAT |
| | | | | TGGCTGCCATAGTTGCCACATTCAACTTCCCGCGGGTCTTCATCTTTT |
| | | | | CTTCATTCCCTAGCACTTCATAGCAACGATGCAAGGTGAATGGCTTG |
| | | | | CCAAGATTCTTGTTGCCTTTCTTGTTCTTCTTTCCCACATCCCCAAACA |
| | | | | ATCCTTGAGCCATATAGTTCTACAAAGCAAGCAAAAGAGAATATAT |
| | | | | GAGCTATATGAACTACAACCTCGTATCCTTCACATATGAGCCATATG |
| | | | | GTCAACATAGTAGTAGAATTGCTTACCCTATCAATCTCATTTGTGCC |
| | | | | ACTTGGGTTAATGACATCAACGTTGGCTTGCATGCCCGCCCATTTTT |
| | | | | GGCAATCGGTGTTGATGCCGGACCAACGAGACCGAAGAGAACGCA |
| | | | | TGGTTCGCTCGTTCCCACTTGTGTTGTTGGCGTCGAGGTACTCCTTC |
| | | | | ATCCTCATCCAGTATGTGTATCTTGTTTGATCGGCACCGGTTGTTGG |
| | | | | ATCCATTCCAATTGTCAACCATGTATTGCAAAGCAACTTGTCCTCCG |
| | | | | CTATGGTGTAGTTCGCCGACCGACCGGGATGGCGAGGTTCAATCAA |
| | | | | CCCTTCTCCTTCCTCATCGATATCCTCATATTCCTCCCATCCCTTAATG |
| | | | | GTTTCATCATGCATAAACGAAGATCCAACATTCATTGCCTCCATGAA |
| | | | | AGACTCATCATCGACTCTACATTGCAATGAAGTTCATTATTCTGTGG |
| | | | | CTATGTATGCATCTAAACTAAAATTTGGATGAAAAGAAGTGTTTTTT |
| | | | | ACCTCTCCGGCATTTTCTCATACACCGTATGTGCGCCGGACGGAGGC |
| | | | | GCCAGCGGCGGCTGGTTCGTCGGTGGAGGAGGCGGCGGAGGCTG |
| | | | | CGCGCGGCGGGTCGCGGTCGCCTTCACTTTCTTCGGCGGCGCAAGG |
| | | | | GAGGCGACGTCCGCAGCTACATACGAGCGTTTGGACGACGACGCC |
| | | | | TTCCTCCTCGGCGGCTCCCGCGCCTTGCCGGGGGCCATCACAGTGG |
| | | | | CCAGCTGCGCAACGGCGGCTGTGTGCAGCGTCGCGCGCAGCGGGA |
| | | | | CGAAGAGAGCACGGGCATCAGCGGTTGTGTTGCCGGCGTCGGTGC |
| | | | | CACCGCTAGGGTTTGGCGCATGGGGCGGCGGCGCGACAGAGGCG |
| | | | | GCAGCGGTGGGTGGAATGGAGTATGGAGGGGTCGGTGGGGTGCC |
| | | | | GACGGGACCGTCCATCGCCGGGATTGTGTCGACGGCCGCGCGGAG |
| | | | | GCGAGGGATTGGGCGGTGCGGTGGCGAGGCGCGGGGGGAAGTTT |
| | | | | CAGCGCGGGATTTGTTCGCGTGCGTCGAGCGATGACGCGTGCTGTA |
| | | | | GCCGGCGTCCAAAGTTTAGCGCTTGGGATAGGGCAAACCGGTCCG |
| | | | | CGTCCCAAATTTTTTTTTACAGCGTTGTAGCATCTGCCGGAGGACCA |
| | | | | ATTTTGCGCTCTCGCGCTGGAAACGACGGTTTATATAGGGCGCAAC |
| | | | | ATATATAGCGCGTGTTGAAGATGCTCTAAGACTGCTCATAGTGGGA |
| | | | | GTAACATTGCTCGTAACGTCACACATCTCAAGGTGTTTTGGTGACAT |
| | | | | GGCATGTCAATAAATTAAGAAAGAGAGTGAAGTGGTAACTAGCTAT |
| | | | | GTTACCNNNACATCACACACCTCAAGGCAAGATAAGTTTACAACAT |
| | | | | GTAATAAATAACGCAATGCATGACACAACATATAAGTTACTACCCAC |
| | | | | TCTATGATAGAGTTTGGCTCTGGTGGTTAGGTCCCTTGTGGTGGAA |
| | | | | CCAGCCCACCCAGGTTCAAGTCCTAGATTTGGTATGGATGTTTGCAT |
| | | | | TTACCTGGATTTATTTCAGAATTTAATCGGCGCTATACTTTCAGTGGT |
| | | | | AGGTGATGTGCCCGTCAATAGCGAGGCGCCAGTGGTGACTTCGTCA |
| | | | | ACCTCAAGATATGGTGGCTCAGTCCCTCGAAGGTGCTCATAGGAGT |
| | | | | AGGGTATGCGTGCGTGCGTTCGTACGGTGTTTGTACGTGCGTATTT |
| | | | | GTGAGTTCTCGTCTGCCTTATACTGTGTTCTAAAAAAAATTCAGTTTT |
| | | | | TATCTTGCCTCGAGAGCCGACTTCCAATCTGGACCCGTTATAGGTGG |
| | | | | ACCCTACGTGTCAATGCCAACACGGCTTTGATTCACGCGCCGCTGCA |
| | | | | ATCTTCATTGTATATCCTTCTATATACCGACGCAGGTTGTTCAAAAA |
| | | | | GAAAATACCGACGCAGGTGGTGAACAGCCTAGCAAATTGTCACCG |
| | | | | ACAGATGGGTCCAAACCAGGATAAGGCCCACATGTCATTGCGTGTA |
| | | | | GTGGCATAACCGGGTTGGCCCACCCCACCCCCAGCCCCATCCCATCT |
| | | | | GAACCACACATCTTGCCACGCCACGCGACTGCACCAAAGACAAAAC |
| | | | | ACGCTCGCACCACCACTCCATCCTCCTCTTTCTCCCTCGCCCAAACCC |
| | | | | TCGCCGCCGCCTTCGCGACATCCGCCGCCGCCATGGCCACTGCCACT |
| | | | | TCCACAGCCGTCGCCTTCTCGGGTGCCACCGCCGCCTTGCCCAAACC |
| | | | | ACGCACTCTCCGGCGTTACCAACTGCTCTCCTCACGCCGCGCCCTCA |
| | | | | CCGCCCCCATCAGGTGCTCCGCGGTGTCCCCTTCGCCTTCGCCCGCC |
| | | | | CCTCCCGCCACCGCGCTCCGTCCATGGGGCCCATCCGAGCCCCGCA |
| | | | | AGGGCGCCGACATCCTCGTCGAGGCCCTCGAGCGCTGCGGCATCA |
| | | | | GCGACGTCTTCGCCTACCCGGGCGGCGCCTCAATGGAGATCCACCA |
| | | | | GGCGCTCACGCGCTCGCCGCTCATCACCAACCACCTCTTCCGCCACG |
| | | | | AGCAGGGGGAGGCCTTCGCGGCGTCCGGGTACGCGCGCGCGTCCG |
| | | | | GCCGCGTCGGGGTCTGCGTCGCCACCTCCGGCCCGGGGGCCACCA |
| | | | | ACCTCGTCTCCGCGCTCGCCGACGCCCTCCTCGACTCCATCCCGATG |
| | | | | GTGGCCATCACGGGGCAGGTCCCGCGCCGCATGATCGGCACGGAC |
| | | | | GCCTTCCAGGAGACGCCCATCGTCGAGGTCACCCGCTCCATCACCA |
| | | | | AGCACAACTACCTCGTCCTCGATGTCGAGGACATCCCCCGCGTCATC |
| | | | | CAGGAAGCCTTCTTCCTCGCCTCCTCTGGCCGCCCGGGCCCGGTGCT |
| | | | | CGTCGACATCCCCAAGGACATCCAGCAGCAGATGGCGGTGCCCGTC |
| | | | | TGGGACGCGCCCATGAGTCTGCCAGGCTACATTGCGCGCCTGCCTA |
| | | | | AGCCACCGGCTACTGAACTGCTCGAGCAGGTTCTGCGTCTGGTTGG |
| | | | | TGAGGCAAGACGCCCAATTCTCTATGTTGGCGGTGGCTGCTCTGCA |
| | | | | TCCGGAGAGGAGCTGCGCCGCTTTGTTGAGCTCACTGGGATCCCAG |
| | | | | TTACAACTACCCTCATGGGTCTTGGCAACTTCCCCAGCGACGACCCG |
| | | | | CTGTCTCTGCGTATGCTTGGGATGCATGGCACTGTCTACGCAAACTA |
| | | | | CGCCGTAGATAAGGCTGACCTGTTGCTTGCGTTTGGCGTGAGGTTT |
| | | | | GATGATCGCGTCACTGGGAAAATCGAGGCTTTTGCGAGCAGGTCCA |
| | | | | AGATTGTGCACATTGACATTGATCCAGCTGAGATTGGCAAGAACAA |
| | | | | GCAGCCGCATGTCTCCATTTGTGCAGATGTCAAGCTCGCTTTGCAGG |
| | | | | GCCTGAATGCTCTGCTAACTGGGACCAAAGCACAAAAGAGTTTCGA |
| | | | | TTTTGGTTCGTGGCATGACGAGTTGGAGCAGCAGAAAAGGGAGTTT |
| | | | | CCTCTGGGATACAAAACTTTCGGGGAGGCCATCCCACCGCAATATG |
| | | | | CTATCCAGGTACTGGATGAGCTCACCAAAGGTGAGGCCATCATTGC |
| | | | | CACTGGTGTTGGGCAGCACCAGATGTGGGCGGCTCAGTATTACACC |
| | | | | TACAAGCGCCCACGGCAGTGGCTGTCTTCGGCTGGTCTCGGGGCGA |
| | | | | TGGGGTTTGGGCTGCCAGCTGCAGCTGGCGCCGCTGTGGCTAACCC |
| | | | | AGGTGTCACAGTTGTTGACATTGATGGGGATGGTAGCTTCCTCATG |
| | | | | AACATTCAGGAGTTGGCGCTGATTCGCATTGAGAACCTCCCAGTTA |
| | | | | AGGTGATGATATTGAACAACCAACATCTTGGAATGGTGGTGCAGTG |
| | | | | GGAGGACAGGTTTTACAAGGCCAATCGGGCGCATACATACCTTGG |
| | | | | GAACCCAGAAAATGAGAGTGAGATATATCCAGATTTTGTGACCATT |
| | | | | GCTAAAGGGTTCAATGTTCCTGCAGTTCGGGTGACAAAGAGGAGT |
| | | | | GAAGTCCGTGCAGCAATCAAGAAGATGCTTGAGACTCCTGGGCCAT |
| | | | | ACTTGTTGGATATCATCGTCCCTCACCAGGAGCATGTGCTGCCTATG |
| | | | | ATCCCTAGCGGTGGTGCTTTTAAGGACATTATCATGGAAGGTGATG |
| | | | | GCAGGATTGCGTATTAATCGGAACTTCTGCAAGAGCTCGACCTACA |
| | | | | AGACCTATAAGTGGAATATGCCTGATCAGCATGATGCTGGTGTATG |
| | | | | TTATATCCATGTGTTCGCTAATTTGCTTGTTTGATGAGCTTGGTTTGC |
| | | | | TAGTCTTACCTAGCTCTGAACCTTCTAGGTTTTCTAGTCTGTTCTTTTC |
| | | | | CGTAGGCATATGCTGTCATAAGAGATCATGCAAGTTTCTTGTCCTAC |
| | | | | ATATCAATAATAAGTACTTTCATGGAATAATTCTCAGTATTTTAGTTC |
| | | | | CAAGTCTTCTCGCCATGTTTGGTTACGTACTAAGTGCATGTGTAAAT |
| | | | | GTATGTCGCCCCGATGGGGGATCAACGACAAGTTCTGGACTGGGA |
| | | | | AATCAGACTGAGTTTGGGTGGAAGGAGAACATACATATGTTCATAT |
| | | | | CATCGTTCCTTTCGTTCAGTAACCATCGACCCACAAGCCATCCTAACT |
| | | | | AACAGACTACGAGACACAGAATTTGAACAATATATGCTACTTGTTCA |
| | | | | CTTGCGTTTGAGGTACGTGATGAAGAATCTCTTGGGAATATGGGTT |
| | | | | TTATTGTATATAAAAGAAGACAATCTTTCTGTGAGATAGAGAATCTT |
| | | | | GAGAATATCCTCATAGATTTTTCAAGGATACTTTTGGATATCATGTA |
| | | | | TTACCTGTAGGCATGCAAGTGCCTGGTGAGTATGCTTAAACCATAA |
| | | | | CAGTTTTAATTTATCATGACAGATCTCCACAAAATAGCCAGAATAGT |
| | | | | TGTGCCTAAAATAAGTAATGTTTTCTTTGTGGCAAGCACAGATGAA |
| | | | | GGTGCCCAACTTTGTTTCACTTTTAGCACATATTTCTCCTTTATTTTGT |
| | | | | AGTCCTCTATTTCTTATTCTTAGGTCTTAACAGATCTTTTGCTAATGG |
| | | | | TCTCTTCGATTTTGAGCTCCTCTTTCTATTTCATAGGATAATATCTTA |
| | | | | GGCTAATGTAATACTGGCCTTGATATGTTTGTGCGTGTGTCTTCTGG |
| | | | | TCTTGCTTACTCAAAAGTATTCGTACCCACGGGTGAATCCAGGAGC |
| | | | | GATGTTGATGCATTCAGCTTTTGCGGATTGATGTTGGTTAGATGGAT |
| | | | | CTGGATGGACCTATGTTAGTTTTAGCTCTGCATGAGTGGTTACCTTA |
| | | | | TATTCCCAAGCTTCATGTTCAGTGGACCTTTTTTTTTTGACCGAATGT |
| | | | | TCAGTGGACCTTGGTGGAGACTCATGTAGCCTCTGTACATTCGTTCT |
| | | | | TGGTATGTGAATTCTTAAATATATTTATATATGAATGCTAAGCTCCAT |
| | | | | GGTGAAGCACGATTCTGTCTTTGATTACTTCTTCGGTGGGGCTGTTG |
| | | | | TTCTCTGTTAATAAAAAGTCTTCACATGTAGCCTACCTGTAAGATATT |
| | | | | GATTGGTGTTGACTGTGCAGGAGCTTAGAGTTTAGGAACCAAGTGC |
| | | | | AAACTTTATTTATAAAACCAAGAATATTAAATAAGGTATTTTTTTTTA |
| | | | | CAAATTAGCATCCGTATCTATCTGGTGTAACAATCGAAGAGAGGTA |
| | | | | TTGTTGTTTTTGTTAAGTTCATACATTTACGTGTTACTTAGATTCACG |
| | | | | CAAATGTACATACTCCATCTGATTGTATCATTTATCGTTTTTGTTTGG |
| | | | | GGTCGCGCAGCTAGCAAGCTGCGCCTTCCTTACATCTGTTGCTCTTG |
| | | | | GCATAACGGTGTTGGTTATTGTAATGTATTTTCCTATTTTCCTGATTG |
| | | | | TATAAGCATTTGGTACTCCTTTCATGATTTTGTTTGCTCTTATCCTAAT |
| | | | | TTCTCTCTTATCTGCTTCGTTTTGTAAAAATTTAAAAACATATTATTAT |
| | | | | TTTCCTGCCTGCTTTTTATTGTCGTTATACGAAATGTAAATAGTGGTG |
| | | | | AATTCCACGGGTACAACTCTTAATCTAGTATCTTTACAGCTGTAAGA |
| | | | | ACATTGCAAGGATTGAATTTCTCTACGGAAATAAAGAATTTTTGAAA |
| | | | | TTTTCAGAATTTCGGGAAGTCACATCAATGATTTTGTCAACCTATTA |
| | | | | ATGAAACTTTCAGGAGTGTACTGTCCTTCATGGTTTCAGATATTTCC |
| | | | | GAACAAGTAAAATATCATGACAGCGGAAGGCTCCCTTGCTGTGCGT |
| | | | | TGCCCTCTGAACTTGTAAGAACCGTATTTAGCATCTTCCTTGCAAGT |
| | | | | GCAGCAATTGATCTCCAATAGCAGCGAGTTAACCATTGACACGGCA |
| | | | | TCAGAAGTCCAAAACTCATAAGTACTGTACTTTTTTTTTTGTAGAAAT |
| | | | | GGCATGCATCCGAGGGTGGATGATGAATTGTACATAGCTTACATTT |
| | | | | TCCTTATTATGTCATGCAGTGCCAACATTCAGAGAAGGGGCTCTATC |
| | | | | ATCATTACGCAAAGCTAATAACTAATAAGCTCCATTTGGTACAGATA |
| | | | | GCCAATGATCAAGCGTCCACGGGATGGGATCTGATTTGTGATAGCA |
| | | | | CATGAGTTGTCTCATTAGAAAATTCGCCACAACTCCCTAGTTTTCCTT |
| | | | | TTCACATCATTTTAGTGTGTGCTGGTCCGGTAGCTCCATAAACAAAG |
| | | | | CGATCATGGACACCGCAAAAATCACAGCTAACCACAGCTTCAAAGA |
| | | | | TCGTGGGAGCCCCCACTGAATCATAATTACATCTACGCTACAGCCTG |
| | | | | AACAGGGTGTCATATACTCGTATCCTGCTGTTTAGGCGCAGCCCAA |
| | | | | AGAACCAAACATCCTTCACAGCTGCAAAAAAGAGATTTGATTTTACT |
| | | | | GAAACCATGTCTACAGAGACATGTTTATGGGTTGCTGAATGACAGA |
| | | | | TCCACTTGGCATGTTTCAAACCCTTATTTATGTATTTACAAACAATAA |
| | | | | AACCTCAGAACTAGGCCAAAACTTCTGTGAGGAATATATCTACAGC |
| | | | | TGTATGTGTTTGACATGATCTATGGTTTTAAGAGGTTCATGGAAACA |
| | | | | TAACAATCATTGTGATAGCTGATAACTGGCAGACAAAAAAAGGGTG |
| | | | | AAAGACCGATTTAACAAGTTGTGAGCAGATCGCAGGCGAGCTCAA |
| | | | | AAGAAGTACAAGCCAGCTCGGGGCACATGTAAAGAGGTCTGATAG |
| | | | | GAGATGATTCAGTACGCATCACCGCAGACGAACTAGATTTACATGA |
| | | | | TTTAACAGTCTAGTAGGAGCATTTGGTACAAAGATGGACCACCCCA |
| | | | | AACCTGGCACCCCTTGACATTCATTCAAGAACTACCTACCCAGAATT |
| | | | | GATAATACCCGTCACCGAAATCTCTTATCCTTTTGGAGGAGGATATT |
| | | | | TGGGTTCCTCTTAGATCCTGTTAGCTCGCCCCATCTCGTCCCTGGTCC |
| | | | | ATGAACGCGTGAAGAAATGCTCCAGAGGGTGTCTCCTTGCTGGCCA |
| | | | | GCCGCATCTTTCGTTGGCTTTGAAATAGCTTTGGATTTCATCAGAGG |
| | | | | ATTCTTGTTGCCTTTACCCATGCCAGCTCCTAGCAGCCATCTTGAGCC |
| | | | | AGCTTGCTGCTGTGTTTGATTACCCCATGGTTTTGTCTTCTCAGGCTC |
| | | | | GCTCTTAAAGCCAGCTACATGCACTTCTGTGACTTTAAACTGAGGGG |
| | | | | CAGAATCTTCCTCCTCGATCTTTTCTTCAGCAACCAATGCCTTATCTA |
| | | | | GAATCTCCTCAGTCTTGGGCTCCTCATAATATTCCTCACTGTTACCTT |
| | | | | TCTCCGAAACAGTGTTGTATATCTTGGGCTTTGGGGGAATAAAAAC |
| | | | | CCTCTCCACTTGAATCAGAGAAAGCATCGGTGTGCCAACTGGCTCAT |
| | | | | AGTTACGTAGCGGGTCACGCAACTGAACCATAAGAGCAACTGTGAA |
| | | | | GTTGTTCCCCAACAAACCCCATCTTCTACCAGATCTTCTGCTTTTCTT |
| | | | | GTCCGCATTCTGGTTTTCAGCAAGTAGGTCCATTGATTTGGCATGGT |
| | | | | GGGCAGCAAGTATTTTCGATGTACGGTCACTGAACTGTTCCTCTTCA |
| | | | | TCGACCACTCCAGAATCAAGTCTCATCCACTCATCTAGAGTGATTGA |
| | | | | TAATCCCATTAGTCCATCAACATCACCACCACTTTGTTTAACATCCAG |
| | | | | AAGCTGCAATCCCGCAGTCCCTTCTAACCCAAGGGTCCGTGTATTCT |
| | | | | CTGCGCATTTTCCTTGCAGAGATGAAATCTCACCAACAGGGTGGGC |
| | | | | ACTGATATTGGAGGGTGCCTCTTCTTCAGACATGCCAGATTGTATCC |
| | | | | TCAGACCCTCAATAGAAAGGGCTTCAATTTTTTCCATAGCTAATGGG |
| | | | | GCAAGGTCCTCAAGTGAAACATATTCCGAAGCATTTATCCCACCCAA |
| | | | | TGATGATGACAGATCAGCACCTCTTCTTTTCTTCTTCTTTCCCAAGGG |
| | | | | AGCATCACTGACCCCTCCTGCTAAAGCTTCCATGCTATGGTTCTCCA |
| | | | | ATAGGTCGTATCTGCATTTGTTAGAGCAATATGTTTTAGCCAGGGTA |
| | | | | AATGGTTAAGTAGGAAGTCATGAGCAAGCATGACAAGAAAGATTT |
| | | | | CTAACCTTCCAGCAGACTCTAGAGCAGGACAAGCCCCCCAGGCGAG |
| | | | | CTGCTGCATCATTTTGCCATTCACATCTTCCAAGGGCATAAGTTTGTT |
| | | | | TGCTTGCATTGATAGCTTTTCGATTCCAACTGAAGCTAGACCGTGCA |
| | | | | ATATCTCCATAATTCCAGATCCCATTTCTGCTGGTAGCACGATTGGA |
| | | | | GAAGAAGCCTGCATGACCAGGCTACAGTTGTTCTTTGCATTCTTGAA |
| | | | | AAGAGCTGGATTCATCGACCGCAGAAATCCTCCATCTTCTGTCTGAA |
| | | | | TAAATGGGCCCAAACCATCAGCCAAAGGCGGCAGCTCGAGGGGCA |
| | | | | GCTCAGGTGGTATGTCAATGGGGCTACCAAACCCACTTCTAGTTCCA |
| | | | | CGTGGGGAAGATTGGAAGGACTTCTCATTTAGCCCGAACTGACGCA |
| | | | | TCAAAGCTTCAGCTTCCTGGTCTTCCAGTGACTTGGCTGTAAATGTG |
| | | | | GCGTCTATGGGCGGAGCTGCACTCTGCAGCTCAAGCTCAGCCTCGT |
| | | | | GTATGATTGTGGAGAGATCAAATTCCTCTGCAAAGTCGTCACAACT |
| | | | | AGCTTCGTCCGCTCCATCTTCGAAATCCAAACCTAGAATACAGTCCC |
| | | | | CAGATTCCAGAGCTTCTTTTTCGAATTGCTTCCAAAGCTTCTCCCTCG |
| | | | | GCGACTCTGAATCACTGTCTGTGGTTGGCCCAAATGGGCTATGCTC |
| | | | | GATCCCAAGCATTTCCAAAAACTCGCTAGCAACAAAATCAGACGAA |
| | | | | GCATCCACGCTGCGTGATCTGCTCTTCCTGCTGGCGGAATTGTAACC |
| | | | | GTCTTCCGCGCCCATGCAGCTCATTCGCCTAGACAGCTTATCGTCCA |
| | | | | CGGTTGGCGAATCAAATTCCTCTGGCTCCACGATTGCGAATTTGTTG |
| | | | | AACAAGGACTCCAGATCCTCCAGCTCTGCATCCTCTGCCAGCTGACC |
| | | | | ATCTTCTTCCAATGATGAAGTAGGGAGCAAGGCTGCTTCGACCGGC |
| | | | | GCATCCTGCTCGTCTCCCACATCGTCCGTTTCAGGAGCAACATCGCT |
| | | | | GGGCTCCTCTTGCAGCTTCACTTCAACGGTGCCGTCTTTAGCAGGAT |
| | | | | CGTCGCTTAAAAATGCAGGTTTGGTCGTTGCCTCTTCATTAGCTTCC |
| | | | | CCATTAAAATTCTCTGCACGGTCAGCCACATAGTCAGCCTTGGCAGG |
| | | | | TTTGAGCTGCTCTAGGTCCTGGAGGGCGACCTCAACTCCCTGCTCAA |
| | | | | CCACACTGAACTCTGCGCTATAGCAATCGCCATCTGGATGCGCAAA |
| | | | | GTCCCCCTCCCCCTTCTTCACCTCCACCGACGTGCAATGCTTTGACTC |
| | | | | CGGCGAGCCATCCTCCTCGGGTTCTACTGCTGCCTTTTTTGTGGCAT |
| | | | | CAAGACCTGCGTCACCAGCAAAGGCCAAGGCTTTGGCAGGCCTGGT |
| | | | | GGTGCTGGGCAGAACCTCGTGCAGCACCCTGACGACGTCCCGGGTC |
| | | | | CGTGCAGGCACGGGCGTGGGCGCCTGCCGCGCCACGGAGCCCCGCC |
| 45 | Lolium | gDNA | 206 | GGGTGACAAAGAGGAGTGAAGTCCGTGCAGCAATCAAGAAGATGC |
| | multiflorum | Contig | | TTGAGACCCCTGGGCCATACTTGTTGGATATCATCGTCCCTCACCAG |
| | | | | GAGCATGTGCTTCCTATGATCCCTAGCGGTGGTGCTTTTAAGGACAT |
| | | | | TATCATGGAAGGTGATGGCAGGATTGCGTATTAATCGGGACTTCTG |
| | | | | CAAGAGCTCCACCTACAAGAC |
| 1692 | Abutilon | cDNA | 1599 | CATGGCGGCTCTCTCGACTCCTCGCCTACCAGTTCACTGCTTGAAGC |
| | theophrasti | Contig | | CCGAGTCAGACTCAGCTCGGAGACCCATTTCTTTCCCTGTAAAGATA |
| | | | | CGAAGACCCATCAGCCATGTCCCCAAGAAACTCGTGCTTTCTCCCAG |
| | | | | GAGCGTCGACGATAAGATCTCTGGCAACAACATCCTTTCCAACGGC |
| | | | | CCCGTTCCTTCAACTCCCACTCGTTCAAAGGTGAGAAGGCATACGAT |
| | | | | TTCTGTGTTTGTTGGAGATGAAAGTGGAATGATCAATAGGATTGCT |
| | | | | GGAGTTTTTGCAAGGAGAGGGTATAACATTGAGTCCCTTGCGGTTG |
| | | | | GTTTGAACAAAGATAAGGCACTCTTCACCATAGTTGTCTCTGGTACT |
| | | | | GAAAGAGTGCTTCAGCAAGTTGTGGAGCAGTTACAAAAGCTTGTGA |
| | | | | ATGTTTGGAAGGTTGAAGATCTCTCTAATGAGCCTCAGGTTGAACG |
| | | | | TGAGCTTATGCTGATAAAAGTGAATGCGGATCCAAAGTTTAGAGCT |
| | | | | GAGATCATGTGGTTAGTGGACATCTTCAGGGCAAAGATTGTGGACA |
| | | | | TCTCAGAACACTCACTAACTATTGAGGTAACAGGAGATCCTGGGAA |
| | | | | GATGGTTGCTGTGCAAAGAAATTTAAGCAAGTTTGGGATCAAAGAA |
| | | | | ATTGCTAGAACAGGAAAGATTGCCCTTAGAAGGGAAAAAATGGGT |
| | | | | GCATCTGCTCCATTCTGGCAATTTTCAGCAGCTTCATATCCTGATCTT |
| | | | | GAAGAAACAATACCTGATAACACTCTTGCAGGGGCCAGAGATAGA |
| | | | | ACAGTAGTTAGCGAGGCTGATGTTTCTGGAGGGGGGGATGTTTATC |
| | | | | CAGTGGAGTCACCTGATGGTTTTACGATCAATCAAGTTCTTGATGCT |
| | | | | CATTGGGGTGTTCTCGTTGATGATACAAGTGGACATCAATCTCATAC |
| | | | | TTTATCCATGCTTGTAAATGACTGTCCGGGAGTTCTAAACCTTGTCA |
| | | | | CAGGTGTTTTCGCTCGAAGAGGCTATAATCTTCAGAGTTTGGCTGTT |
| | | | | GGACATGCAGAAGTTGAGGGGCGCTCTCGCATTACCACTGTTGTTC |
| | | | | CGGGTACAGATGAATCAATTAGCAAGTTAGTGCAGCAACTATATAA |
| | | | | GCTCGTAGATATGCATGAGGTACAAGATCTTACACCCCTGCCATTTG |
| | | | | CTGAACGAGAATTAATGTTGATAAAGATTGCCGTGAATGCCGCTGC |
| | | | | TCGGCGAGATGTCCTTGACATTGCCACCATTTTTAGGGCCAAGGCT |
| | | | | GTTGATGTTTCGGACCACACTGTCACTCTCGAGCTTACAGGAGATCT |
| | | | | AGACAAGATGGTTGCGCTGCAGAGATTGTTGGAGCCTTATGGTATT |
| | | | | TGTGAGGTTGCTCGAACTGGACGTGTAGCGTTGGTGAGAGAGTCT |
| | | | | GGTGTCGACTCCAAATATCTACGAGGATATTCTTTTCCACTTTAAGTT |
| | | | | CCAAGTAGTATTTCTAAGGTATTGGTGTGAGACTATTTTTGCGTTGT |
| | | | | TTTGTTTTGATTACCAAACAGATAAGTTTAGATACTTCAGAGCTTTG |
| | | | | ATAATCATAAAGTTTTGGGAAGTTTGAATAATCAAAGCTCCACTGAA |
| | | | | ATATATTCTTGTTATGTGTA |
| 1693 | Amaranthus | cDNA | 714 | ACTCGCTGTTGGATTGAACAAGGACAAAGCTCTCTTTACTATTGTAG |
| | graecizans | Contig | | TTTCTGGAACGGATAATGTGTTGCAGCAAGTGATGGAACAACTTCA |
| | | | | AAAGCTTGTCAATGTTTTGAAGGTTGAAGATATATCCAAGGAGCCT |
| | | | | CAAGTAGAACGTGAATTGATGCTTGTAAAAGTTGGAGCTGATCGGA |
| | | | | ATAACCGTGCTGAGTTGATGTGGTTGGTGGGCATCTTTCGTGCCAA |
| | | | | AATAGTGGACATATCGGAAGAGTATCTTTCAATAGAGGTCACTGGA |
| | | | | GATCCAGGAAAGATGGTTGCTGTCCTCAGAAACCTAAGCAAGTTTG |
| | | | | GCATCAAAGAAATTGCTCGTACCGGAAAGATTGCTCTAAGAAGGGA |
| | | | | AAAATTGGGTGAGTCTGCTCCTTTCTGGCGTTTTTCTGCTGCTTCTTA |
| | | | | TCCTGATCTTGAAGAAGCTATCCCTATGGATGCTCTTTCTGGAGTTT |
| | | | | CAAAAAGAGCAGCTGCTGCTGGATCATCAGATTCGTCTGTGGAGGG |
| | | | | TGATGTTTATCCTGTGGAGCCGTTTGATGGTTTCTCCCCTCCAATCTT |
| | | | | AGATGCTCATTGGGGTATTTTGAATGAAGAAGATACTAGTGGGATG |
| | | | | CGATCACACACTCTATCTATTCTTGTCAATGACAAACCCGGGGTCCT |
| | | | | TAATGTTGTTACGGGGGTTTTTGCTCGAAGGGGTTATAACATTCAG |
| | | | | AGTTTAGCTGTGGGTCA |
| 1694 | Amaranthus | cDNA | 632 | GTGTCGACTCACCTTTCAACGAGTTTTAACTCCATTCCGAAAAGCAA |
| | hybridus | Contig | | TAGATTGAACCACCAAACTGCAAACGATTAGGGTTCTCCTTGAAACC |
| | | | | CCATTCTCTGGGTTTTAAGTTTAACTCCAATAGTGACAGGAATTCGG |
| | | | | AGTTTGATAAACTGGTTGTATCTGCAAGCAATGTTGATCAACTGGG |
| | | | | AAATCAAAGTAACTTATCCTTTAATCCCCCTTCTCCCTCTCGATCAAA |
| | | | | GGAGAGACGACATACAATATCAGTATTTGTGGGGGATGAAAGTGG |
| | | | | AATGATTAATCGAATAGCAGGGGTTTTTGCCAGAAGAGGTTATAAT |
| | | | | ATCGAATCACTCGCTGTTGGATTGAACAAGGACAAAGCTCTCTTCAC |
| | | | | TATTGTAGTTTCTGGAACGGATAATGTGTTGCAGCAAGTGATGGAA |
| | | | | CAGCTTCAAAAGCTTGTCAATGTTTTGAAGGTTGAAGATATATCCAA |
| | | | | GGAGCCTCAAGTAGAACGTGAATTGATGCTTGTAAAAGTTGGAGCT |
| | | | | GATCGGAATAACCGTGCTGAGCTGATGTGGTTGGTGGACATCTTTC |
| | | | | GTGCCAAAATTGTGGACATATCGGAAGAGTATCTTTCAATAGAGGT |
| | | | | CACTGGAGATCCAGGAAAGATGGTTGCT |
| 1695 | Amaranthus | cDNA | 495 | AAAGAAATTGCAAGGACCGGTAAGATTGCTCTAAGGCGAGAAAGA |
| | hybridus | Contig | | TGGGTCAGACGGCTCCTTTTTGGAGATTCTCTGCTGCTTCTTATCCA |
| | | | | GATCTACAAGAAAAGGCTGTTGATGATCTGGCCAGGCCTACGAAAC |
| | | | | GGAGCATCAATGGTGATTCTGGCTCATCTTCAAGTGGTGATGTTTAT |
| | | | | CCGGTGGAACCTTATGATGGTTCGATGGTAAATCAAGTACTTGATG |
| | | | | CTCACTGGGGCGTACTTTATGACGGTGATTCCAGTGGTCTCCGGTC |
| | | | | ACATACGCTGTCCATGCTGGTAAATAATGCTCCCGGAGTTCTTAACA |
| | | | | CTGTTACAGGAGTAATTTCTCGTAGGGGTTATAACATTCAGAGTCTT |
| | | | | GCTGTAGGCCCTGCTGAAAAGGAGGGTCTTTCTCGTATCACAACTG |
| | | | | TCATTCCTGGAAACGACGAGTCGATTGGAAAATTGGTTCAGCAATT |
| | | | | CAACAAGTTAGTAGACCTTCATGAGATTCAGG |
| 1696 | Amaranthus | cDNA | 1326 | GCAATAGATTGAACCACCAAACTGCAAAACGATTAGGGTTCTCCTT |
| | lividus | Contig | | GAAACCCCATTCTCTGGGTTTTAAGTTTAACTCCAATAGTGACAGGA |
| | | | | ATTCGGAGTTTGATAAACTGGTTGTGTCTGCAAGCAATGTTGATCAA |
| | | | | CTGGGAAATCAAAGTAACTTATCCTTTAATCCCCCTTCTCCCTCTCGA |
| | | | | TCAAAGGAGAGACGACATACAATATCAGTATTTGTGGGGGATGAA |
| | | | | AGTGGAATGATTAATCGAATAGCAGGGGTTTTTGCCAGAAGAGGTT |
| | | | | ATAATATCGAATCACTCGCTGTTGGATTGAACAAGGACAAAGCTCT |
| | | | | CTTCACTATTGTAGTTTCTGGAACGGATAATGTGTTGCAGCAAGTGA |
| | | | | TGGAACAGCTTCAAAAGCTTGTCAATGTTTTGAAGGTTGAAGATAT |
| | | | | ATCCAAGGAGCCTCAAGTAGAACGTGAATTGATGCTTGTAAAAGTT |
| | | | | GGAGCTGATCGGAATAACCGTGCTGAGCTGATGTGGTTGGTGGAC |
| | | | | ATCTTTCGTGCCAAAATTGTGGACATATCGGAAGAGTATCTTTCAAT |
| | | | | AGAGGTCACTGGAGATCCAGGAAAGATGGTTGCTGTCCTTAGAAAC |
| | | | | CTAAGCAAGTTTGGCATCAAAGAAATTGCTCGTACCGGAAAGATTG |
| | | | | CTCTAAGAAGGGAAAAATTGGGCGAGTCTGCTCCTTTCTGGCGTTTT |
| | | | | TCTGCTGTTTCTTATCCTGATCTTGAAGAAGCTATCCCTATGGATGCT |
| | | | | CTTTCTGGAGTTTCAAAAGGGGCAGCTGCTGCTGGATCATCGGATT |
| | | | | TGTCTGTGGAGGGTGATGTTTATCCTGTGGAGCCGTTTGATGGTTTC |
| | | | | TCCCTTCCAATCTTAGATGCTCATTGGGGTATTTTGAACGAAGAAGA |
| | | | | TACTAGTGGGATGCGGTCACACACTCTATCTATTCTTGTTAATGACA |
| | | | | AACCTGGGGTCCTTAATGTTGTTACGGGGGTTTTTGCTCGAAGGGG |
| | | | | TTATAACATTCAGAGTTTAGCTGTGGGTCATGCGGAAGGTGAGGGT |
| | | | | CTATCTCGTATCACTACTGTTGTACCCGGTACAGATGAATCAATTAG |
| | | | | CAAATTGGTTCAACAAATCTACAAGCTGGTTGATATTCATGAGGTTA |
| | | | | GAGATCTTACCCATTATCCATTTGCTGAGCGAGAGTTGATGTTGATA |
| | | | | AAAGTAGCTGTGAATACTGCTGCACGTCGTGAGGTCCTAGACGTTG |
| | | | | CCAGCATTTTTAGAGCAAAAGCTGTTGATGTATCTGATCACACCATA |
| | | | | ACACTTGAGCTCACTGGAGATTTGAACAAGATGGTTGCTCTACAGA |
| | | | | GATTGCTCGAACCGTATGGAATCT |
| 1697 | Amaranthus | cDNA | 621 | CTGGAAATGACGAGTCGATTGGAAAATTAGTTCAGCAATTTAACAA |
| | lividus | Contig | | GTTAGTAGACCTTCATGAGATTCAAGACCTTACTCACCAGCCATTTC |
| | | | | AGAGCGAGAGCTTATGTTGATCAAAATAGCTGCAAATACTACAGCC |
| | | | | AGGAGAGATGTCCTTGATATTGCTAATATTTTCCGTGCAAAAGCTGT |
| | | | | GGATGTTTCTGATCATACAATAACATTACAACTTGCTGGTGATTTAG |
| | | | | ACAAAATGGTTGCGCTACAGAGATTATTGGAGCCTTACGGCATTTG |
| | | | | TGAGGTGGCACGGACTGGAAGAGTAGCACTAACGAGAGAGTCTAG |
| | | | | GGTAGATTCCAAATATCTACGAGGATACACTCTTCCGTTGTATGAAT |
| | | | | GAAAAACTCCAGCTATGTCTTTCCGACATTACCTTGTCTGATTCCTTC |
| | | | | CGCTCTTCTACACTGCATTGCAAGCAGAACAATTGCCCACAAGTGG |
| | | | | AGTAAATTAAAAGGGGAAACAACAATACCGATCTTTGCTTTGAAGA |
| | | | | TATACTGATTTGTATAACAATAGAGTTTGTATTTGACTAGTATTTATT |
| | | | | TGCTCAACGCTGTAATAACATATCCCCTTGAGGTTTGATATATGGAA |
| | | | | GGAAAAATTACCCAG |
| 1698 | Amaranthus | cDNA | 509 | TTCATTTGTTTCTCATCTCTCTCCAATGGCGGCCATTTCCTTTAACATC |
| | lividus | Contig | | AATGGCGGAAAGATTGGAACTTTATGTCCAAAACCTAAATATGGTT |
| | | | | GTGCGTTTTTGAGAAAATGGGATTTTGGAGCTCATACAACTGTATAT |
| | | | | ACTAAACCCATGTCAAAAATTTCAAGCCTGAAAGCAGTTGAAGTTTC |
| | | | | TACCAATGCTACAGAAAACGCAGTTTCTCTTTCAGCTAACTCTAGGG |
| | | | | TGATGCATCACACAATTTCTGTCTTTGTCGGGGATGAAAGTGGAAT |
| | | | | AATCAATAGGATTGCAGGTGTTATTTCTAGAAGAGGATATAATATC |
| | | | | GAGTCTTTGGCTGTTTGTTTAAACAAGGATAAGGCTCTTTTTACTAT |
| | | | | AGAAGTGTGTGGAACTGACAAGGTGTTGCGCCAAGTCGTGGAACA |
| | | | | GCTTAACAAGCTTGTTAGTGTTTTGAAGGTTGAAGATCTATCGAGA |
| | | | | GAGCCACAAGTGGAACGTGAACTGATGCTTGTAAAGCTTAATT |
| 1699 | Amaranthus | cDNA | 1678 | AAATTTCCATCCATGGAGGCTGTGTCGACTCACCTTTCAACGAGTTT |
| | palmeri | Contig | | TAACTCCATTTCGAAAAGCAATAGATTCAACCGCCAAACTGCAAAAC |
| | | | | GATTAGGGTTCACCTTGAAACCCCATTCTCTGTGTTTTAAGTTTAACT |
| | | | | CCAATGATGACAGGAATTCGGAGTTTGATAAACTGGTTGTATCTGC |
| | | | | AAGCAATGTTGATCAACTGGGAAATCAAAGTAACTTATCCTTTAATC |
| | | | | CCCCTTCTTCCTCTCGATCAAAGGAGAGACGACATACAATATCAGTA |
| | | | | TTTGTGGGGGATGAAAGTGGAATGATTAATCGAATAGCAGGGGTT |
| | | | | TTTGCCAGAAGAGGTTATAATATCGAATCACTCGCTGTTGGATTGAA |
| | | | | CAAGGACAAAGCTCTCTTCACTATTGTAGTTTCCGGAACGGATAATG |
| | | | | TGTTGCAGCAAGTGATGGAACAGCTTCAAAAGCTTGTCAATGTTTT |
| | | | | GAAGGTTGAAGATATATCCAAGGAGCCTCAAGTAGAACGTGAATT |
| | | | | GATGCTTGTAAAAGTTGGAGCTGATCGGAATAACCGTGCTGAGCTG |
| | | | | ATGTGGTTGGTGGACATCTTTCGTGCCAAAATTGTGGACATATCGG |
| | | | | AAGAGTATCTTTCAATAGAGGTCACTGGAGATCCAGGAAAGATGGT |
| | | | | TGCTGTCCTTAGAAACCTAAGCAAGTTTGGCATCAAAGAAATTGCTC |
| | | | | GTACCGGAAAGATTGCTCTAAGAAGGGAAAAATTGGGCGAGTCTG |
| | | | | CTCCTTTCTGGCGTTTTTCTGCTGCTTCTTATCCTGATCTTGAAGAAG |
| | | | | CTATCCCTATGGATGCTCTTTCTGGAGTTTCAAAAGCAGCAACTGCT |
| | | | | GCTGGATCATCGGATTCGTCTGTGGAGGGTGATGTTTATCCTGTGG |
| | | | | AGCCGTTTGATGGTTTCTCCCCTCCAATCTTAGATGCTCATTGGGGT |
| | | | | ATTTTGAATGAAGAAGATACTAGTGGGATGCGGTCACACACTCTAT |
| | | | | CTATTCTTGTCAATGACAAACCCGGGGTCCTTAATGTTGTTACGGGG |
| | | | | GTTTTTGCTCGAAGGGGTTATAACATTCAGAGTTTAGCTGTGGGTC |
| | | | | ATGCGGAAGGTGAGGGTCTATCTCGTATCACTACTGTTGTACCCGG |
| | | | | TACAGATGAATCAATTAGCAAATTGGTTCAACAAATCTACAAGCTG |
| | | | | GTTGATATTCATGAGGTTAGAGATCTTACCCATTATCCATTTGCTGA |
| | | | | GCGAGAGTTGATGTTGATAAAAGTAGCTGTGAATACTGCTGCACGT |
| | | | | CGTGAGGTCCTAGACGTTGCCAGCATTTTTAGAGCAAAAGCTGTTG |
| | | | | ATGTATCTGATCACACCATAACACTTGAGCTCACTGGAGATTTGAAC |
| | | | | AAGATGGTTGCTCTACAGAGATTGCTCGAACCGTATGGAATCTGTG |
| | | | | AGGTTGCACGAACAGGACGTGTGGCCTTGAGTCGAGAATCAGGTG |
| | | | | TAGACTCGAGATACCTTCGTGGATACTCTTTCCCTGTATAAGAGTTC |
| | | | | TGTCAAAACGTATGATGTAAAAAGCCTTTGGGATTTTTTCTTTTCGA |
| | | | | AATTGTCTCCAATTTTTGATCCTTTGAAGTTGTGTTCTTGAAGAATAT |
| | | | | TTGCAAGTTTGTTATGAAACTTTGTTTAAGTGAACAATAATTCTCTCA |
| | | | | TGAGACGGTCTAATGTGTGAGATCTCTTGTTATATTGGGCTTAGTAG |
| | | | | CCC |
| 1700 | Amaranthus | cDNA | 1483 | ACATATGCTAAAAACTTCAAGGTTTAAAGCAATGGAAGTTTCTGCA |
| | palmeri | Contig | | AATGCAACAGTAAATATAGTTCTGTTTCAGCTCATTCTAGGGTGATG |
| | | | | CGCCACACAATTTCAGTCTTTGTTGGGGATGAAAGTGGGATAATCA |
| | | | | ATAGGATTGCAGGTGTTATTTCTAGAAGAGGATACAATATCGAGTC |
| | | | | TTTGGCTGTTTGTTTAAACAAGGATAAGGCTCTTTTTACTATAGAAG |
| | | | | TGTGTGGAACTGACAAGGTGTTGCGCCAAGTCGTGGAACAGCTTAA |
| | | | | CAAGCTTGTTAGTGTTTTGAAGGTTGAAGATCTATCGAGAGAGCCA |
| | | | | CAAGTGGAACGTGAACTGATGCTTGTAAAGCTTAATTCTGATGCAA |
| | | | | ATTCCCACGCAGAGCTAATGTGGCTAGTGGACATCTTCAGGGCAAA |
| | | | | AATTGTGGATATCTCAGAAAGCTTGGTCACTGTTGAGGTGACCGGA |
| | | | | GATCCTGGAAAGTTGGCTGCTGTCCTAAGAAATTTTAGAAAGTTTG |
| | | | | GAATCAAAGAAATTGCAAGGACCGGAAAGATTGCTCTAAGGCGAG |
| | | | | AAAAGATGGGTCAGACGGCTCCTTTTTGGAGATTCTCTGCTGCTTCT |
| | | | | TATCCAGATCTACAAGAAAAGGCTGTTGATGCTCTGGCCAGGCCTA |
| | | | | CGAAACGGAGCATCAATGGTGATTCTGGCTCATCTTCAAGTGGTGA |
| | | | | TGTTTATCCGGTGGAACCTTATGATGGTTCGATGGTTAATCAAGTAC |
| | | | | TTGATGCTCACTGGGGCGTACTTTATGACGGTGATTCAAGTGGTCTC |
| | | | | CGGTCACATACGCTGTCCATGCTGGTAAATAATGTTCCCGGAGTTCT |
| | | | | TAACACTGTTACAGGAGTAATTTCTCGTAGGGGTTATAACATTCAGA |
| | | | | GTCTTGCTGTAGGCCCTGCTGAAAAGGAGGGTCTTTCTCGTATCACA |
| | | | | ACTGTCATTCCTGGAAACGACGAGTCGATTGGAAAATTGGTTCAGC |
| | | | | AATTTAACAAGTTAATAGACCTTCATGAGATTCAGGATCTACTCACC |
| | | | | AGCCATTTTCAGAGCGAGAGCTTATGTTGATCAAAATAGCTGCAAA |
| | | | | TACTACAGCCAGGAGAGATGTCCTTGATATTGCTAATATATTCCGTG |
| | | | | CAAAACCTGTGGATGTTTCTGATCACACAATAACATTACAACTTGCC |
| | | | | GGTGATTTAGACAAAATGGTTGCGCTACAGAGATTATTGGAGCCTT |
| | | | | ACGGCATCTGTGAGGTGGCACGGACTGGAAGAGTAGCACTAGCTC |
| | | | | GAGAGTCTAGGGTAGATTCCAAGTATTTACGAGGATACACTCTTCC |
| | | | | GTTGTATGAATGAAAAACTCCGGCTATGTCTTTCCGACATTACCTTG |
| | | | | TCCGATTCCTTCCGCTCTTCTACACTGCACTACGGGCAGAACAATTG |
| | | | | CCCACGAGTAGAGTAAACTAAAAGGAGAAGCAACAATACCGATCTT |
| | | | | TGCTTTGAAGATTGTATAACAATAGAACTGTCTCTTATACACATCT |
| 1701 | Amaranthus | cDNA | 1480 | ACATATGCTAAAAACTTCAAGGTTTAAAGCAATGGAAGTTTCTGCA |
| | palmeri | Contig | | AATGCAACAGTAAATATAGTTCTGTTTCAGCTCATTCTAGGGTGATG |
| | | | | CGCCACACAATTTCAGTCTTTGTTGGGGATGAAAGTGGTATAATCA |
| | | | | ATAGGATTGCCGGCGTTATTTCTAGGAGAGGATACAACATAGAGTC |
| | | | | TCTGGCTGTTGGTTTAAATAAGGATAAGGCTCTTTTTACTATAGTAG |
| | | | | TGTGTGGGACTGACAAGGTGTTGCGCCAAGTAATGGAGCAGCTCA |
| | | | | GCAAGCTTGTTAATGTCTTAAAGGTTGAAGATCTATCTAGAGAGCCT |
| | | | | CAAGTGGAACGTGAACTTATGCTTTTAAAGCTTCATTCTAATGCAGA |
| | | | | TACCCATGCTGAGATAATGTGGTTAGTGGACATCTTCAGGGCAAAA |
| | | | | ATTGTCGATATGTCGGAAAGCTTCGTTACTGTAGAGGTGACTGGCG |
| | | | | ATCCTGGAAAGATGGCTGCTGTCCTGAGAAATTTTAGCAAGTATGG |
| | | | | AATCAAAGAAGTTGCCAGAACAGGAAAGATTGCTCTAAGACGAGA |
| | | | | AAGGATGGGCGAGACAGCTCCTTTTTGGAGATTCTCTGCTGCATCTT |
| | | | | ATCCAGATCTAGAAGAAAAGGCTGTTGAATCTTTTGTCAGGCCTGC |
| | | | | AAAAGAAGCATCAATGCTGATCCTGGCTCATCGTCTAGTGGTGATG |
| | | | | TTTATCCAGTGGAACCTTATGAAGCCTCCATAAATACAGTACTTGAT |
| | | | | GCTCACTGGGGAGTTCTTTACGAAGATGATTCAAGCGGACTTGTGT |
| | | | | CACATACTCTGTCCATGCTGGTAAATAATGCTCCTGGAGTTCTGAAC |
| | | | | ACCGTTACAGGAGTAATTGCTCGTAGAGGTTATAACATTCAGAGTC |
| | | | | TTGCTGTGGGCCCTGCTGAAAAGGAGGGTCTTTCTCGTATCACAACT |
| | | | | GTTGTTCCTGGAAACGACGAGTCAATTGCAAAATTGGTTCAGCAAT |
| | | | | TAAACAAATTAGTAGACCTTCATGAGATTCAGGACCTTACGCACCA |
| | | | | GCCATTTGCAGAGCGAGAGCTTATGTTAATTAAAGTAGCGGCAAAT |
| | | | | ACTTCGGCCAGGAGAGACGTCCTTGATATTGCTAATATATTCCGTGC |
| | | | | AAAACCTGTGGATGTTTCTGATCACACAATAACATTACAACTTGCCG |
| | | | | GTGATTTAGACAAAATGGTTGCGCTACAGAGATTATTGGAGCCTTA |
| | | | | CGGCATCTGTGAGGTGGCACGGACTGGAAGAGTAGCACTAGCTCG |
| | | | | AGAGTCTAGGGTAGATTCCAAGTATTTACGAGGATACACTCTTCCGT |
| | | | | TGTATGAATGAAAAACTCCGGCTATGTCTTTCCGACATTACCTTGTC |
| | | | | CGATTCCTTCCGCTCTTCTACACTGCACTACGGGCAGAACAATTGCC |
| | | | | CACGAGTAGAGTAAACTAAAAGGAGAAGCAACAATACCGATCTTTG |
| | | | | CTTTGAAGATTGTATAACAATAGAACTGTCTCTTATACACATCT |
| 1702 | Amaranthus | gDNA | 9758 | ATGGTAAATCATCACTATGCGGCCTAATTTGATGTGGCCTAATTTGA |
| | palmeri | Contig | | TAAAAGGATATTTGGTTTTTTTTTTTAATTTTTTTTCTTGTTTTAATTA |
| | | | | TTTTATTAAAATGACTGATGATTTTAAATCACCATTATTGATTAAGAT |
| | | | | TTAATATAAATCACATTGTAAAAGCAACACAAAAAGCACAAATTCAA |
| | | | | TAATATACTCTTTAAGTTTGTTTATCTTCTAATTAGTTCGGTTAAAAC |
| | | | | GGTTCCCCACTTTCTTCTCCGACTCTCACAATTATCTTCCCCTATTCAT |
| | | | | TTTTCTTCCACCCTCTCTAATGGCGGCTGTTTCCTTCAATATCAATGG |
| | | | | TGGAAAGATTGGAACTTTATGTTCAAGACACGAATTCGTTTGTGGG |
| | | | | TTTGTAAGAAAATTTCATTTTAGAACTCATACTTCTATATTTGAAAAA |
| | | | | CATATGCCAAAAACTTCAAGGTTTAAAGCAATGGAAGTTTCTGCAA |
| | | | | ATGCAACAGTAAATATAGTTCCTGTTTCAGCTCATTCTAGGTAATTTT |
| | | | | ATTTCTCGAAAATTTTTGATTTACAATTAAATTAATCTTGTTTTGTAG |
| | | | | GTAATGAATTGCAGATGAAATAGATGGATTCTTATTTGTTTATTGGT |
| | | | | ATTTGTTTATAAATTTTTGTTTATATTAGTTTCTGAATTGTGATTATTC |
| | | | | TGATTGTATGTCAAGGTTTAGGTTGTTACTAAATGTAAATTGGATTG |
| | | | | ATTGAAGTTGCAATAAGGTGATGGCGTGATGCTGATTGTTGTAAAT |
| | | | | TTTTGTTTATATTAGTATAGTACTACTTATTGACCTAAGTTCTTTTACA |
| | | | | TAGTAGGAGATATTATAACTTTTGCTTGTGGACTCAGGGCCGGCCCT |
| | | | | AAGGGTGGGCAAGAGGGGCCGTCTCCCAATGCCCATGTCAAAAAA |
| | | | | GCAAAATTAATGGTTATATAAGGTTTATATAGGTTATAAATTGTAAA |
| | | | | GGAAAAGGACGTCAATGTGTCATTTCTCCCAGGGCCCCAAAATATC |
| | | | | TAGGACCGGTCCTGAAAGAAACTACTCAAAATTTGTAGGATTTTCTA |
| | | | | TTGAGACATTTTTTTTTTTGTGGATTAATGATTGAACTAAGATCAAAT |
| | | | | AGATACTATTTCTGTTTGATTGAAACTTTTGGGTAGCCGGATGTGTA |
| | | | | CCGGTGTGTGACTTCTTTTTGAGTTGGTTAGTTCAATTGTATTGTTA |
| | | | | GAAATGATCTTTTCTAGGGTCTTTGGAGACATTGGTTCTTTACTTTTA |
| | | | | GTCTATGAATTATGATAGTGTTGGTTTAAACACCATTTTGTTACCCTA |
| | | | | TTGTGATTAAGTGACTCCCAGCTCGATTAAATTTAGGAGTTATATTA |
| | | | | ATGTTGTCTTTGTTAGTTATATTGATGGTCGGTAGTTCGGTTAAGTT |
| | | | | TGTCTATGAATACATTGATTTTATCCTTATTTGGCCAACAAATGTATG |
| | | | | TAATTTAGGAAAGTTATATGATTTACCACTTTGTATTTGTATCTCTGT |
| | | | | TATTTTCTTTCTTCTTTTCTTGATGTGTTGGTTGGATAAAAGGAATTT |
| | | | | GGAGGGAAAGAAAGGAAAGAGAAATAGAGGGATGAGACTTTGTC |
| | | | | ATATGTATATCAAATAGAGATGGGAAATGGAGGGGAGGGAATAAT |
| | | | | AACAATGAATTCCTTCTATTTTTTTTGAAACCAATCCCTCCATTATTA |
| | | | | GAAAGTTTTATATTAAAAACTCACCGCTTTCCCTCCCCTACTCCTTCC |
| | | | | TTTCTAGTCTCTCCCCCCTCCCACCCTTCAATAGCTTATCCTTTCATGC |
| | | | | CGTCCAACAAAATTTGAACTCATATCCTTGTGTATGATCGGAGAAAG |
| | | | | TAATGACTGAATCATTCATGAAGCTCATCCTTGGTGAATTTATCTCTT |
| | | | | TTTATCCAGTCTTTGAGTCTCGTGGTTCAGGAACGCTGCCAAATCTC |
| | | | | TTGAATAAAAGTCATTTTAAATTATCATATGTATCACATTCTATGCTG |
| | | | | TTTTTTTTTCTTGTATTTTGCTATCATTAGAATGGTAATAAAATGTATT |
| | | | | TTCATTCTTTTCCAGGGTGATGCGCCACACAATTTCAGTCTTTGTTGG |
| | | | | GGATGAAAGTGGTATAATCAATAGGATTGCCGGCGTTATTTCTAGG |
| | | | | AGAGGATACAACATAGAGTCTCTGGCTGTTGGTTTAAATAAGGATA |
| | | | | AGGCTCTTTTTACTATAGTAGTGTGTGGGACTGACAAGGTGTTGCG |
| | | | | CCAAGTAATGGAACAGCTCAGCAAGCTTGTTAATGTCATAAAGGTA |
| | | | | GTCATCTCCTTTTTTCTTACTTTAAGGTAACTAGTGATACTTTCATTTA |
| | | | | GTAGTGGCGGTCAATGGTGATTCTAACCATGCATGTTCATATTGTTT |
| | | | | GTGCAATATTGCTCATTTTGAGTCTTTGACTTTATCATTGTTGTTAAA |
| | | | | CTTTGAGAGAATATAATCTCGAGAGCATCTATCCTCATTTTAAGATA |
| | | | | GATAGAGCAGGAAAACACTCATGATTCTTTAATAAACTTTCAAAATA |
| | | | | TTAAAATTCCCTTGCCCTAATGCAATTTTATAGCTCCCAGCTAATCCG |
| | | | | AGTTCAAGGGGTTCGAGTGTATGCAACCATACTCATATAACAACAA |
| | | | | AATGGTTGATTCAGATTGACCCTTAAAATCAAATACCATCTTGCAAA |
| | | | | TTCACTTAAACACATACTTTAACGACCCGTTTTATAGTAGTCCATTAA |
| | | | | AATTCGAAAGCAAAGAATTATAAAATCTTTTATTCCATCGAGAATAG |
| | | | | ACACAATAATCTTCAAAGCAATGTCTATAAAAAGTTTGTCGTGTCAC |
| | | | | CGTATCGTAACTTAATTGTAAGGGCCTTCAATTTTACTGCCATCAAA |
| | | | | ATATAGGTTGAGACCCACAAAACCATTTACATAGACAAGAGCCATT |
| | | | | TCACTACCATTATCATAACCATGATTGAGACCGCATTTGTGCACAAT |
| | | | | GGTCAAGTGACATTCTTCAGCAATACTTAGAGCTTGAATTGGAGGA |
| | | | | ATTTGTATAATGATCTACTTATGTAACAAATTCTTGATAATGTTGTCA |
| | | | | CATATAGTGGGGAGAAATTTGAAGGTCACAAAGCTACCACATGTAA |
| | | | | CTGAATGCATCTATTGCAGTCAAACCTGATGGCAGACAACTGTCAA |
| | | | | GGCAGTCTTTTGTAAGGAACCTTTAGAAGGTATCTTTTAAAGAAAA |
| | | | | AAAATTTGTCGTTAGTTTTTAAAAAAAGTTAAAAATTATTTTCTTGAA |
| | | | | AATTTTCTTTTTAAGTAAAATAAGTGTGGTAAAATGGACATTATGTT |
| | | | | GGAACTTAGAATTTTTAGTAATAGCTTGTTAATTAGCATGGGAAGT |
| | | | | GATTAATGAAGTTTTATGTCCATTAGGTAATAATGTGTGAAGTATTC |
| | | | | TTATATGTATTATTGCTGCACTTTCCATTTGATGGTGCGCATCAGGG |
| | | | | CATGCAATAGTACTTTTTTTGATCTAACATATATGTCCTTACTTATGC |
| | | | | TATATGCTCGCAGCTAAGTATTCCTTACTAAAGGTGTTTTACGGGGC |
| | | | | GATTCGACTAACGGGTTAAGGGTCGGGTCACATTTTAACGGGTCAT |
| | | | | CAACGGATTAGGTTAATAAAGGGTCGGGCCATTAACGGGCCTTGGT |
| | | | | GTAAAGGGTTGGGCAGAGCCGGGTCAAATAATTATAGGAATTGGT |
| | | | | TGCAACATTTTTTTTTTCCACTTTTTGTTTTTATATTTTAGATATTATAT |
| | | | | ACAAAGTTAGGTTGACCCAATGGGCCATAAAGTTGAATAAAAAAAA |
| | | | | CTATTACATGAACCTTTAAAAACGGGTCAAAGTGGGCCTGGTTTAA |
| | | | | ACGAGCTTTGACCCGTCCTGCCCCGTTGAAATTTGACATGACCCTCC |
| | | | | CCGCCTCGCCCTATTTAAATTTAGCTCCATCCCGGCCCATTGAACAC |
| | | | | ATCTATTCCTAAAATAACTAACATTATGATGGCTCATCCTATTCGCTA |
| | | | | CCATGCATGTAAATGTACACAGTGTCTATCCGTTTATCCTTTTTTATA |
| | | | | TCCCTGTATACCATTTGTGACTAACGGTATTAATTTTTTATCTTCCTT |
| | | | | GTAGGTTGAAGATCTATCTAGAGAGCCTCAAGTGGAACGTGAACTT |
| | | | | ATGCTATTAAAGCTTCATTCTAATGCGGATACCCATGCTGAGGTAGT |
| | | | | TCCACAAGTTCAAATAATTATATCCTGAATGCTATATAATTCATGAAT |
| | | | | CTTTTGTTTTCCTCTTAGAATCATCAGCTACAAGAAGGTAGATAAAG |
| | | | | ATAAGCAAATATTCAAAAATATGCTATAACATCATGTGCTTTGTAAG |
| | | | | CTTTAAACTTAGAGATAACAAAAATACTTAAGAATACATCATGGGTA |
| | | | | TGTAGCTGAAGATCAAATGGGAAGTTACATTGCTTCTTTGTGTGTAT |
| | | | | TGTAAACAATCTGGTATCGTATAAGTCCAAATTCCATTTTTTGATTTT |
| | | | | CAGATAATGTGGTTAGTGGACATCTTCAGGGCAAAAATTGTCGATA |
| | | | | TGTCGGAAAGCTTCGTTACTGTAGAGGTTAGTATGTTTGTGGTTGA |
| | | | | CATCTTGTGTACCTATCTTCCGTTCAATGTAGATTCTTTTTCTTAGTG |
| | | | | CTATTTTATTTTCTGTCGTGCAGGTGACTGGCGATCCTGGAAAGATG |
| | | | | GCTGCTGTCCTGAGAAATTTTAGCAAGTATGGAATCAAAGAAGTTG |
| | | | | CCAGAACAGGAAAGGTCAGTCTTTTAGACAATTGCAGTAAATTCAC |
| | | | | CGGAATACTATGAATGATGCAAAGATTACGTGCGGTAAATCGTAAA |
| | | | | CAGGCTGTTGAATCTCTTCAACTAAATTATATCTGATGCGAAGTGCG |
| | | | | TTTATTCAACTACCTTTTTTGTGTGAAATGATAGATTGCTCTAAGACG |
| | | | | AGAAAGGATGGGCGAGACAGCTCCTTTTTGGAGATTCTCTGCTGCA |
| | | | | TCTTATCCAGATCTAGAAGAAAAGGCTGTTGAATCTTTTGTCAGGCC |
| | | | | TGCAAAAAGAAGCATCAATGCTGATCCTGGCTCATCGTCTAGTGTA |
| | | | | AGTGAAGTTTTATTTGATCAACATTTATCTTATCCTTCTCTTACTTTGT |
| | | | | CTTGCATTAGCGTATATTCTTTTTGGTAATTACAAATTTAGTTTTTTTT |
| | | | | AAATGTTCCTAGGGTGATGTTATTATTGACATTGATAAATATTCTTAT |
| | | | | TGGTAGTATATAGATATATTATATTTCCCATAGATTGCTCTAAGACA |
| | | | | GGTGGCGTATATCCGCCCGTCCGAAACCCCGCCAAGTCTCATACGG |
| | | | | GCATACGGCCCGTTTGGTAACTGGCAATGATAATGAATGTTAGTAT |
| | | | | AATTTTGGTAAGAATATCTTCTCATAAACTTAATGACTTTACTTATTC |
| | | | | TCCTTCAACAAGCTCATTTTCCTTACAAAATTCTTTATAATGCATCAC |
| | | | | CACTTAAACCTGTGGTATAAGGTGGTACTGAAATATTGTGAACAAA |
| | | | | AACACTTTTTTATTATTAAAATTTCATTACCATAGTGATGACATGATA |
| | | | | CATATTATGAAGATTTACACTACAAATCATTCCCATTACCACCATTTA |
| | | | | GAACCATTAGCCTAACGGGCAGTTAATGCATTGGGATAATGGAGTG |
| | | | | GAAAAAACGTTAATTGTCATTTATGAACTCAATTTTTTGTTTTGGGTT |
| | | | | TGGCACTTTTGCATTTGGAATTTAAAATGCGTTTATTCAATGAAGGG |
| | | | | TGATGTTTATCCAGTGGAGCCTTATGATGCCTCCATAAATACAGTAC |
| | | | | TTGATGCTCACTGGGGAGTTCTTTACGAAGATGATGTAAGTTCTCTT |
| | | | | TGTCACTACCTGAATTTTGTTCATCTTGTAGCATACTTTCATATGATG |
| | | | | TGATCTTCAGATCTTTTACTTAATGTTACCTTAATTTACTAAATGGCT |
| | | | | TCATATTTTTCCCAGAATGGCCTAAAATTCGTTAGTTTGGCTCATAAT |
| | | | | TATTTAAGACATGTCAAACTTTCCTTAAGACCGGCTCTATCAAATCTT |
| | | | | TAGCCTTATTTGGGAGGTTCTGGTTAATGTCTTTCAATGTTTGGCTT |
| | | | | ACTTGTTCATCCTCAGAACTTTCATATTTGTTTGATTTTAAGCAACGT |
| | | | | TATTAGAATTGAGATTCTACAACACTACGATTCTACCTACGATCTAG |
| | | | | ATTGCTCGCTTGTTTCGGATCGTATTATAGTAGAATTGTAGATCAGA |
| | | | | ATCATGATTTTAATAACTATGGTTTTAAGAATGGAGTAGTTTAATAA |
| | | | | ATCTGTTCTGTTAGTATTCTGATGTACTTTCAACATGCCAGTCAAGC |
| | | | | GGACTTGTGTCACATACTCTGTCCATGCTGGTAAATAATGCTCCTGG |
| | | | | AGTTCTGAACACCGTTACAGGAGTAATTGCTCGTAGAGGTTATAAC |
| | | | | ATTCAGGTCAGTACGGACTCTGACTGCACCATGAAAGTATATCTCGT |
| | | | | CTTTTGCATTATGTTAAGTTGATTGTCCCTTTTCCTTCTTCCCAGAGTC |
| | | | | TTGCTGTGGGCCCTGCTGAAAAGGAGGGTCTTTCTCGTATCACAACT |
| | | | | GTTGTTCCTGGAAACGACGAGTCAATTGCAAAATTGGTTCAGCAAT |
| | | | | TAAACAAATTAGTAGATCTTCATGAGGTGAGCATCTATTTGCATTCA |
| | | | | GCGTAGACATAATCGTCCAAATTAGTTAGTGTCATGTTTACTTATGT |
| | | | | CGTTGTTGTCGCAGATTCAAGACCTTACTCACCAGCCATTTGCAGAG |
| | | | | CGAGAGCTTATGTTAATTAAAGTAGCGGCAAATACTTCGGCCAGGA |
| | | | | GAGACGTCCTTGATATTGCTAATATATTCCGTGCAAAACCTGTGGAT |
| | | | | GTTTCTGATCACACAATAACATTACAAGTAAGCATAAATGAATTTGG |
| | | | | TAGTTTATTTAGTCTTCTCTTGTTACTCTCAACAATGTCTATTCACAAA |
| | | | | TTCTTGTGTGAGACGGTCTCACCGTGATACGTTCCCCATACATGGAT |
| | | | | TGAATAGCCCAACTAATACAATTATCAGCATATGAGCTTCAATTAGT |
| | | | | CTCTTACAAGAGTAAATCATGTCTATATCCTTATTTCGATGGTCCATT |
| | | | | TCGAATTGAAACGAATATAAATAAAAAGCTAATGGAATGCGAGTAT |
| | | | | TTCACTAATAACGAAAACCGTAGGGAGCTAAGACAGTTTACGGTAT |
| | | | | TGAGGATCTCTGTAATTTGTCTTTGTTTTTTTCAGCTTGCCGGTGATT |
| | | | | TAGACAAAATGGTTGCGCTACAGAGATTATTGGAGCCTTACGGCAT |
| | | | | CTGTGAGGTATGTTTATATGCATATTTATTTCTCCCGAAACCGATTA |
| | | | | GAATCATTTCATGTGTTCGGGTCAAATAGGAAATATATGGCCATTTG |
| | | | | TAGCTTTTCAATAGACTCCGAGAAGGCGAGAACCCTTATCTCCTTAA |
| | | | | CTACTAAAACATTTACAGCTCGTTGATTGTCGGTAATAAAAGGTGAT |
| | | | | ATTGAGAATAATTTGTAGTGTAAATTTTTCATGATTGTATTATATCAT |
| | | | | TCCCTGGGTAATGAATAGTTAATCATAAAAAAACTTTTTTTTTCGCA |
| | | | | ATTTTTCATTACCATTTGTGTCACCTCTACGAATGGTAATGCATATGA |
| | | | | TTGATTGAATGAGCTTTATGAAGGAAATAAGATGATTGAAGTAAAT |
| | | | | CAATCAAGATCGTTAAACTTGTAAAGAGATTTTCTCACCAAACTTAC |
| | | | | TCTAAATTTCATTTCCATTTCCACTATTCAATACCGATAACTAAACGG |
| | | | | GCATCAGCACCATTTAGTCGAGTACTTGATTGCAATTCAATTTAATT |
| | | | | CAGTTCAGTTTAGTTGTAAGTAGTTCTAGAGATAGCGAACTTCAATG |
| | | | | ATCATGGACTGACTATAACATTTTATATCTGTCTTCAAGGTGGCACG |
| | | | | GACTGGAAGAGTAGCACTAGCTCGAGAGTCTAGGGTAGATTCCAA |
| | | | | GTATTTACGAGGATACACTCTTCCGTTGTATGAATGAAAAACTCCGG |
| | | | | CTATGTCTTTCCGACATTACCTTGTCCGATTCCTTCCGCTCTTCTACAC |
| | | | | TGCACTACGGGCAGAACAATTGCCCACGAGTAGAGTAAACTAAAAG |
| | | | | GAGAAGCAACAATACCGATCTTTGCTTTGAAGATTGTATAACAATA |
| | | | | GAGTTTGTATTTGACTAGTATTTACATTCTTTTTATTAAATTGTATGA |
| | | | | TTTGCTCAACGTTGTAATTACATATCACCTTGAGGTTTTTGATTTAGC |
| | | | | ATTGTACCCTGGCTAAGATAACATAGTCATTTCAGGACATACGAGT |
| | | | | GTTCCCAGAACACAAGGAAAAAAAAAAGAACACAAGAATATTGAA |
| | | | | AATTTGAAACAAATAACTAATTTAAAAAAAAAAAAGCTTCTGGTAA |
| | | | | ACTTAATTTCAAAATAAAGAATTTCCATGTCCAAGCCTAAGGTCATG |
| | | | | TATGTTCAAATTCAACTTTCAAGTAAGACCTTATCAAAAATTATCAAT |
| | | | | ACAAGTTTATCAATATTTTATATTATTAGTGTTATTAGGTCAATTGGC |
| | | | | TCCATTTTTACGGGCTTGGTTAAAGGACTCAATAGTTCAGTCAACAC |
| | | | | CTTAGGAAAGCCCACCTTAGGGCTGAAATTACAAAAATGCCTTTTG |
| | | | | AATAGTCATACAGGTCATTAATTGCTTTATAAGTACCCCCATTAATT |
| | | | | GTCGTTTATGGTATGCTTTCTCTCTCTATATTACTCACTTGCATTCAAT |
| | | | | TCCTATTTCATTGTAACGATCACTGACTTGAGCGTCGGAGGGGCTTT |
| | | | | CGGAGAGACCACCCCCGGACAAGTAACTCTGTTCGTTTTGCTGGTC |
| | | | | ATCAGTCGAACTCGATTTTAGTATTCGGACCCTCCAACAGCAGGATC |
| | | | | CACATTCACAAACAACCCCTTCCAATGAGGAATAATTTGACTCGATT |
| | | | | TCTACGCAAAAACAATTAGCATGGAAAATATTTATATATACTAAAAT |
| | | | | ATTATACATTGTAAAATAAATCATTTAAATTATACAACAATGTGGGT |
| | | | | ACAAATTAAAATGTAATTTCATTTCAAGCAATTGGTAGGTTGAATGA |
| | | | | AGCCTAAAACATTCCCATGAGCATTTTGACAATATGATTGAGCTTTT |
| | | | | CCATCAATGTATTTGAGATCAATATTTTGGAGTTGAATTCCTTTGCAT |
| | | | | GGATTTGTGGAACTACAATTAAATGTCATTGCTACCTCGCTTTGTGA |
| | | | | ACTTCCTCTTATATTTTGGTACTTCACATCCTCGATTTCTATACCCGAT |
| | | | | TCCTAATAAACAAAATTAATTATATCATTTATATCTCTGATTGTGCAT |
| | | | | AATTAAAAATTACAAAAAAATTAATATTAGGGATCTTTGCATTGAAA |
| | | | | CGAATCAAACAAGCTAAGATCCCACTTGATTACATATTGTCAAGGA |
| | | | | ACCAACTCAACCAAAAGCTTAAGCTGATTGTTGAGGCCTTAGGATA |
| | | | | TAGTATATACTCTAACACGCCCCCTCACACGAGAGCCCTTTGGGCTT |
| | | | | GAAGTGTGGACGCGACATAGAACCTCCTAATTCCTGGCGTTGAATA |
| | | | | TTCCACTTTAAATGAGGGGTGATTGAGATTCGAACCTGTGACATCTT |
| | | | | GTTTCACTGGCTTTGATACCATGTCAAGAGACCAACTCAACTAAAAG |
| | | | | CTTAAGCTGATGGTTGAGGCCTCAGGATATGTTATATACTCTAACAC |
| | | | | ATATTGTTTATAGATTAAGAATAGTGTTTGGCAAATTAGCTTATTGA |
| | | | | GCCATTTTAGCTTATTTTGATTCAAATAGAGGGTTGAGTGGCAAACC |
| | | | | AGCTAATGCTAGTGTTTGGTGAATTAGCTTGTTGAGTCAGCTTATTG |
| | | | | ATATGAATAAGCTGTTTTTGAACAATCTGCTCATAGCAACGGGTTCA |
| | | | | AAAACAGCTGGTCAAACCAGTTAATATAATCAGTTAGTTAAATCATC |
| | | | | CACTATAATCAACTATTAGTTATCAGTCGTTTGCCAAACATTCTCAAA |
| | | | | ATATAAATTACGAGTAATCGATAACAGTGCCGTTTCTGGCAATGTAC |
| | | | | GGGCC |
| 1703 | Amaranthus | gDNA | 7941 | CAAACAAAAGGTATAAATTTAATTAATATACTTAATTCAAAAAAAAA |
| | palmeri | Contig | | AAATTAATTAATATACTAGTAAGTGTGTTTCGCATTTCAATATTTGAA |
| | | | | TAATATTATCTTTTTGGAATGATAATATTTAAAAGCAACAAACAAAA |
| | | | | GTTATAAATTTAATTAAAAATTACAACTAAGTGTGTTTTGCATTTCAA |
| | | | | TATTTGAATAAAAAATTATAAGTGAGTTGTCTATTTATAATAAAATA |
| | | | | AATAATGCACTACTCATATAATCATATTCATCCTCACTACTCATATTC |
| | | | | TTCTTCAAAAAAAATCCTCACTACCATCTCCATTCTCCCCATTTCATTT |
| | | | | CTTTCTCATATCTCTCTAATGGCGGCCATTTCCTTTAACATCAATGGC |
| | | | | GGAAAGATCGGAACTTTATGTCCAAATCCTAAATATGGTTGTGGGT |
| | | | | TTTTGAGAAAATGGGATTTTGGAGCTCATACAACTGTATCTACTAAA |
| | | | | CCCATGTCAAGAATTTTAAGCTTGAAAGCAGTTGAAGTTTCTGTTGA |
| | | | | TGCTACAGTAAATGCAGTTTCTGTTTCAGCTAATTCTAGGTAATGTA |
| | | | | CTTAATTAATATTACCATTTTTTCATTGAATCTAAACTTTTTTTCTTCA |
| | | | | TGTGGAATTGTGGATATAGGAGTATTTATGGAAAGAAATATAAGG |
| | | | | GTTTTTGCTTCTTTATTGGTTTTTGTTAAAAAAATTGTATGGTTGCAT |
| | | | | ACTTTTGTGATCATTCATGTTCAACACGTTCGATTGTACAAACTCTAA |
| | | | | AAAATTGAATGCAAAAGATACAAAATTGTTCGAAAATATCATAATTT |
| | | | | TTGAAATTTCACAGGGTCTTTTAAAGAATTTTTCAAATTTTGCTCCGG |
| | | | | TCCTGATTCTCCTAAGCTCACCCTATAAGGACTTAAGGAGTCACCAA |
| | | | | AAAGTAAAGTTGAGTATACTAGTCATGTGACTCTAATTTTAGCCTAG |
| | | | | GTGGAAGAAATAGGGTCAAGGTCAATGGAATGTTGTTTGTTTGTTT |
| | | | | GTTCTGGTATCATTACAATTTACAGATATGAAGTTGCAATAAGGTTT |
| | | | | TGGATTATATTGTACCATATTAAATTGGCTTATTAGACTATGTGTACT |
| | | | | TTTATTTATATGAAATTGAGATGATGTTTGCTGCCAAACGTCAATCG |
| | | | | AAAACAACTATATATTATCATTAACAAGGGTAAATGTGAGGTATTG |
| | | | | ATCCCCAAACCCCCACAAATGGAAGTTATTTGATGGCATTGAGGTG |
| | | | | ATATAACATTGTATGAAGTTGCAAATTATGAAGCTAAATCCTGTATT |
| | | | | AAGTGGGAGATAGAGCATCTTTTGGTTGTAATGTTATAACTTTAGTT |
| | | | | GGATTTTGTAAGTTTTGTTGCAAGTTATTCATGAATTAAGGAGATGT |
| | | | | GTCTTTCTATGTGAATCACATATGTAAGAAGTAGTTTATATGTTTGTT |
| | | | | GATCTTTATACTGTATGATGGCCGATTCAAATGTGGAAGTTGATATG |
| | | | | ATCCTTTTAAGGTCTACTGAGAAGTTGTTCTTCAATTCATTATATTAT |
| | | | | CCTTGGTTTTCGACCATAGGAGGTCACATGTTCGAATCTAGCCTCAT |
| | | | | ATATAACATATCTTGAGGCTTCAACCATCAACTTAAACTTTGATTGA |
| | | | | GTTGCTTCCCTGTGACTTTCTCGCCTTCGCTTCACCTTTATGCTTGGT |
| | | | | TTCAGCCTTGCTAAGTTGCTAGTCGTGGAGAGGGTTACTTCAAGAA |
| | | | | GGGTGTCAGCATATATCACTGCAGATTTTTTTGAATACAAAATCGTT |
| | | | | TTTAAAGAAATCATACATGGTTATCTTACTGCTCCTTGATTCGATGG |
| | | | | AGCAAAGTTTTGTTGTTATTTGATTTCCGATTATAATGGATCATTGTA |
| | | | | AAACGACTCTTTAAATTCTGCGCACTTCTTATTTATGTAAAGTTGCAA |
| | | | | ATGATGTTTGCTATATATGTGGTTATCTTATTGTATCTTGTTATTTATT |
| | | | | CTCAGGTAATGAAACAGAATTCAAACTCTAATGACATTTCATATATT |
| | | | | TCATTTTGTGTATACAGGGTGATGCGTCACACAATTTCTGTCTTTGTC |
| | | | | GGGGATGAAAGTGGGATAATCAATAGGATTGCAGGTGTTATTTCTA |
| | | | | GAAGAGGATACAATATCGAGTCTTTGGCTGTTTGTTTAAACAAGGA |
| | | | | TAAGGCTCTTTTTACTATAGAAGTGTGTGGAACTGACAAGGTGTTG |
| | | | | CGCCAAGTCGTGGAACAGCTTAACAAGCTTGTTAGTGTTTTGAAGG |
| | | | | TAGTTAATTTCCTTTGTTCTTCCTTTTATTGGTAAATGGTGATAGTGA |
| | | | | TACCCTTGTAACATAAATCTTCGAATAATGAGACTATGACATAGTGC |
| | | | | TTGGTTCACCACTTTACGGATTGCGTTCGATAAAATTACTTTACGAG |
| | | | | GTCTTATAATTTATAAATCTGTTTTCCAAAATAGCTTATACAAACTAA |
| | | | | ACATATCCTGAATTTGTTTTTTACTAAAATAAAAAAAAATTCTTTTAA |
| | | | | TATGGAGCGTTACGTATCAATCAATATAATGGATCATCTTATATGCA |
| | | | | TATGTTTGTACAATGTACATTTTTTGTTAGATTGTCTATTTGTCCTGA |
| | | | | TAACAACCTTGGATGATTCTTGTATTTCTTGTAGGTTGAAGATCTATC |
| | | | | GAGAGAGCCACAAGTGGAACGTGAACTGATGCTTGTAAAGCTTAAT |
| | | | | TCTGATGCAAATTCCCACGCAGAGGTAGTTCCAAAAATTCAATGATA |
| | | | | ATTTATGAATGGTATATTTTCCACTTTTTAAGTTCTAATTTCCTCGGC |
| | | | | TGTTGTCAGTTAGAGTCTAGCAAGGAAAAAGATAGAGATGTTTTGC |
| | | | | TTTAGATTTTATCGATGATCGAGAAAGATGCTATACTTCATATAAGG |
| | | | | CCATGACTGTTACAACAACAACGCCAGAGCTTTAGTCCCAAAAGTTC |
| | | | | ATGGCTGTTAGATAGTTTAAATTTAACTAGAAGTACATCGTGTTGTG |
| | | | | TGAAGCTCGTATAAGAAATTGCATTAGTGTCTTTGTGAGAAATCTAA |
| | | | | TACCGATTAACGTAAATTTTTAATTTTTGATCTTCAGCTAATGTGGCT |
| | | | | AGTGGACATCTTCAGGGCAAAAATTGTGGATATCTCAGAAAGCTTG |
| | | | | GTCACTGTTGAGGTTAGTGGTATATTTGTTTGACTTCTCGCGTATCT |
| | | | | CTTCTATGTTTGCTGAATTAAGTCCGATTTTTTTGGATTTGCTTCCAT |
| | | | | GATAAATGCAAGAAATTTACCTACAAGAAGTTGACCATTTTCCTCCG |
| | | | | GTCGATGTAAAATCTTTTTCCTTATTGGCATTTTCTTTGGTGCTGTGC |
| | | | | AGGTGACCGGAGATCCTGGAAAGTTGGCTGCTGTCCTAAGAAATTT |
| | | | | TAGAAAGTTTGGAATCAAAGAAATTGCAAGGACCGGAAAGGTTAG |
| | | | | TCTTTTAGACATTTGCAGTAAATTCAGCATAGAATTCGGAAAGCCGC |
| | | | | AAAGAGCACATGTCAACTATTGTGGCTAGCCAGCCAAGTGCCAACT |
| | | | | ATTGTGCATTTCTGGTGTGATCAATGTGAAACAAGTTTAGCTAACTA |
| | | | | CTTGTTTTGTTTTATACTTAATATGATAGATTGCTCTAAGGCGAGAA |
| | | | | AAGATGGGTCAGACGGCTCCTTTTTGGAGATTCTCTGCTGCTTCTTA |
| | | | | TCCAGATCTACAAGAAAAGGCTGTTGATGCTCTGGCCAAGCCTACG |
| | | | | AAACGGAGTATCAATGGTGATTCTGGCTCATCTTCAAGTGTAAGTG |
| | | | | ATATTTTATCTTCTCGACAATTATCTTTTTCCTTTTCTTACCTTGCATT |
| | | | | GCCTATGTATACTTCGTTCACCTTGTATTGCCTATAGACTTAAACATG |
| | | | | CTTTTATTTTTGAAGGGTGATGTTTATCCGGTGGAACCTTATGATGG |
| | | | | TTCGATGGTTAATCAAGTGCTTGATGCTCACTGGGGCGTACTTTATG |
| | | | | ACGGTGATGTAAGTGCTCTATCACTACCTGAATTTTTTGTCACTAGG |
| | | | | TTGTCCAACGGTCCATGTGATGTCATGTTCAAATCGCTTAGCCTAGT |
| | | | | GTTTTCTAATATAGCTCCAAATTAATTGTCTGGATTCACGATTCACCC |
| | | | | AAAATGGCCTAAATTTTTTCGTTTTAATTTACAATTGTCCAAGTCATG |
| | | | | TCAAACATGTATTAAGGTCTACACTCTGCTCTATGTTACTTGGACTC |
| | | | | GGGTACTGAGTCGGCAACGTGTCCAAGTGTCGTACGCGTCTAAATA |
| | | | | TTCAATTTTACGCTTAAAATGAAGTGTCTAAATGTCATACCAATGTC |
| | | | | CGGGCATCAAGGATCGGACATAGGTACGTGAAGCAAAATGAAGAG |
| | | | | TCTGAGTAATAGCGTTTCAGCCTCATTTGAACTTTGGAGGGTTAAAG |
| | | | | TTTATGTTTTTGGGTTTTCAGATGTTCATCTTTCTGTTATTCATGTTTA |
| | | | | ATTTTAAGAATGAAAGGTAGTTCGACTTTTCAGTTTTTTGAGCTGCA |
| | | | | CAGTACTATAAATTGGTTTTAGTTTGCTCTGCTAGTATCATGAATTGC |
| | | | | TTCGTTCTTCATGCCAGTCAAGTGGTCTCCGGTCACATACGCTGTCC |
| | | | | ATGCTGGTAAATAATGTTCCCGGAGTTCTTAACACTGTTACAGGAGT |
| | | | | AATTTCTCGTAGGGGTTATAACATTCAGGTCAGTGTGGACTCTGACT |
| | | | | ACAGCAAGAAAGTATATAAAGCCGTTTGTGTTATGTAATGACAATC |
| | | | | GTTCCCTTTTCATCCTCTTCAGAGTCTTGCTGTAGGCCCTGCTGAAAA |
| | | | | GGAGGGTCTTTCTCGTATCACAACTGTCATTCCTGGAAACGACGAG |
| | | | | TCGATTGGAAAATTGGTTCAGCAATTTAACAAGTTAATAGACCTTCA |
| | | | | TGAGGTGAGCAACTATTTATGTACAAAACCGAATAGTTGTAATAAG |
| | | | | TGGTATAGACTACATAGTAATAACTTTATGGTTGTAAGATGCAAAGT |
| | | | | ACCCTTATCAGTTATCACTATAATATGTTCTAAGAAGCTCAAAACTG |
| | | | | AAGTGGAAGAGAAAATTGTGACATTGGTTAGCTCCGCGTTTATGAC |
| | | | | AAACTTCTTGATTGATTCTTGTTTTTTTGAATGCTCAGTTCAATCTCCT |
| | | | | TTGTAATATGTTGATGCTCGTTAAGTTACCTATTTTCTTTGTAATACG |
| | | | | TAAACCGTGAAGTTGTGTTATCTTAGTCCAACGTCACATCGTGCTGC |
| | | | | TCGAGTCATTTGATTTAAAACGCTATTTATATTGAATAGTGTAACAT |
| | | | | ATAACACAGAAAAATAGTAGTCGGGTTGGTTCAACACACACAATAA |
| | | | | TTTCCCACATTCTCCCTTGAATGAAAGATACGAGAATCTAACATCAG |
| | | | | AATGGTTGCTGCTGCAGATTCAGGACCTTACTCACCAGCCATTTTCA |
| | | | | GAGCGAGAGCTTATGTTGATCAAAATAGCTGCAAATACTACAGCCA |
| | | | | GGAGAGATGTCCTTGATATTGCTAATATTTTCCGTGCAAAAGCTGTG |
| | | | | GATGTTTCGGATCACACGATAACATTACAGGTACTAAGCTGTAGGC |
| | | | | ACACAGTTTTTGCCATGTTCCTTGTTTTTTCCTAATATTTCTGACTTGG |
| | | | | TCTTTTCCAGCTCGCCAATATGTTGGGATTCAGGCTTTGGCATTATTT |
| | | | | ATTATTTTATTTTTTTTGCATTTTTTCCAACTTCCTTATATTGGGATTA |
| | | | | AGGCGTTGGCGTTGTTGCATTGTTGACTGTTGTGTCTTTTCCAGCTT |
| | | | | ACGGGTGATTTACACAAAATGGTTGCGCTACAGAGACTATTGGAGC |
| | | | | CTTATGGCATTTGTGAGGTATGCGCCTATGTATTATTGATTTTTCTAT |
| | | | | ATAATCAGATTTTATTTATTTATTTTTGGTTCGAAATATGCTGATTGT |
| | | | | TAGTTATTTTGCTTTTGAATGTGAAAGGATTCTCTTGTCTGTAGATTA |
| | | | | AGAACAATCCTGCCAAAAATACCTATTGGGCTTACCTTAACTAATTA |
| | | | | CATCTGTCTTGAAGGTGGCACGGACAGGAAGAGTAGCACTGAGTA |
| | | | | GAGAGTCTGGGGTCGATTCTACATCTTTACGTGGATACGCTCTTCCA |
| | | | | TTGTACGAATAAAAAATCTCCGGCTATGTCTTTCCGTCATTATCATGT |
| | | | | CCAATTTCCTTCAGCACGTCTGCACTACATTTACATAGCGACCAATT |
| | | | | GCCCGTCAACCAGAGTGAAGGAAAGCAGTAATTCTGATCTTTGCTT |
| | | | | AGAACATGCTATTTTGTATAATAGGAATAATTTTGTATTGGTTGTAT |
| | | | | TTGCTCAACACCCTGTAGTAACATTATTACCTCGAGTTCGAATATAT |
| | | | | GAAACATAGTTTTTCTCTTAAATGCGTTGAGAACATTAGTACGGCCC |
| | | | | CTTGGCCGGCTGACACGAATTAATCGAGAAGGAATTAGGACTGTAA |
| | | | | ACAATTCCAAATACTAGTGTTTTCTCGTACTGGGAAAACAAAAGCTT |
| | | | | TTACTCGACTTCTTAGCTTTCGTAGTAGCATTTTGCAATAATTTCCGA |
| | | | | AATCATAACTTCAACAAACTCGCCATCACAATTCACACCGAGCAATG |
| | | | | TTTGATGCATCGATCTTATAATCAAACGAGGATATACAATGGGGCA |
| | | | | TAGGATAAACACAGGAAACTATAATTTTATTACGAAAAATTCTACTA |
| | | | | TAAGCCTTTATAACGATCAATTACACATTAAACCTACTATAGAGGAT |
| | | | | GATCTATAAGTTTGTATATCACACAGAATCTAACAGCTCATTCACCA |
| | | | | ATTTCTCATTCCCGAGCGACAATGAGCTCAGAAGGAAAACCAGCAT |
| | | | | CCGAGGAATCTGTAGGAACCAGTATATCATCATCGGCTGTGAAGTC |
| | | | | GTAGTTTTGACCGATGGCTCTCAATAGTTGATACACTTCGAACATTG |
| | | | | TTGGCCTTTCCTTGTCGTTTAAGTTAACACAGTTACAAGCAACTTTAA |
| | | | | GGAACTGTGTTAGTTCGTTATCGACACTCTTGTCACGGAGAGACTTA |
| | | | | TCGATGGCAGAACGGCGATCGTTTTTTTCTGATAGTAGATGTATCCA |
| | | | | TTCTACCAAATTACCCTTGAAGGTTTCGGGGGCATTCGACACGTGA |
| | | | | GTTGGTTTTTCACCCGTGACTAGTTCCAAAAGAACAGTGCCGAAACT |
| | | | | ATAGACATCTCCCTTTGGAGTTGCTACAAGTTTTCTCGGGTATTCGG |
| | | | | GAGCTACATACCCAAGATCACCAAATTCTCCGTTAACGAAAGTGCTC |
| | | | | ATGTGGGTGTCAATCGGGTTCATGAGTCTTGCAAGACCGAAGTCGG |
| | | | | AGAGCTTGGGATTGAAATCCTCGTCCAACAAAATACATTTCGAGCTT |
| | | | | ATGTTTCTGTGGAGGATTCTAGGGTTGCAATTATGATGCATCCAAGC |
| | | | | CAACCCCTTAGCTGCGCCGATAGAAATTTTAAGCCTCAAAGGCCAAT |
| | | | | CCAAGCTCTTAGCCTCAGGATCTGAAGGGTGAAGCCTATCGTAGAG |
| | | | | GTTTCCGTTTGCCATATATTTGTAGACCAAGAACCTCTCTTTCTTTGC |
| | | | | TGTGCAGAAGCCCAACAGCGGTACCAAGTTTTGGTGCTTCACACTTC |
| | | | | CGAGAGTGTTCATCTCAGACAAAAATTCTTTCTCTGTCCGCTGTGAA |
| | | | | TCTTGCAATCTTTTAACCGTAAGGAGCGTTCCATCAGGCATCATCGC |
| | | | | CTTGTACATCGTTCCCATTCTTCCAGAACCGATCATATTGTTGTTGCT |
| | | | | GAAGCTATTAGTGGCTTTCATGAGGTCATCCAAACTCATTTTCGAAA |
| | | | | TTGTCTTCTCGAACATAGAAACCTGAATATCAAGAAAAACATTCTAC |
| | | | | ATTACACACGCGTATAAAGAATGGAAAATCAGTAACAAACTACTCC |
| | | | | TGTCTCACACTACAAGTCGCATACATAAATATAGTATTGTCGGATTT |
| | | | | CAGCAAAGAGTTATTGTTATCGGTCAAACAGGAGAGCTCACCAAAA |
| | | | | GACTAGCTTATTAGGTGAGAGCCATTTGCCTATAAACCCCACACCAA |
| | | | | TTGCATACACAACCGATGTGGGACAAGTTCCATACCCTGCAATGGA |
| | | | | CCATAAATCGACCCCCATAAGGCCAACGTCCTTGTTGGGTCACGGG |
| | | | | ATGACACAACACAGAGCCTACAACCAACAACAAACA |
| 1704 | Amaranthus | gDNA | 5448 | GGTCACATTTTAACGGGTCATCAACGGATTAGGTTAATAAAGGGTC |
| | palmeri | Contig | | GGGCCATTAACGGGCCTTGGTGTAAAGGGTTGGGCAGAGCCGGGT |
| | | | | CAAATAATTATAGGAATTGGTTGCAACATTTTTTTTTTCCACTTTTTG |
| | | | | TTTTTATATTTTAGATATTATATACAAAGTTAGGTTGACCCAATGGG |
| | | | | CCATAAAGTTGAATAAAAAAAACTATTACATGAACCTTTAAAAACG |
| | | | | GGTCAAAGTGGGCCTGGTTTAAACGAGCTTTGACCCGTCCTGCCCC |
| | | | | GTTGAAATTTGACATGACCCTCCCCGCCTCGCCCTATTTAAATTTAGC |
| | | | | TCCATCCCGGCCCATTGAACACATCTATTCCTAAAATAACTAACATTA |
| | | | | TGATGGCTCATCCTATTCGCTACCATGCATGTAAATGTACACAGTGT |
| | | | | CTATCCGTTTATCCTTTTTTATATCCCTGTATACCATTTGTGACTAACG |
| | | | | GTATTAATTTTTTATCTTCCTTGTAGGTTGAAGATCTATCTAGAGAGC |
| | | | | CTCAAGTGGAACGTGAACTTATGCTTTTAAAGCTTCATTCTAATGCA |
| | | | | GATACCCATGCTGAGGTAGTTCCACCAGTTCAAATAATTATATCCTG |
| | | | | AACGCTATATAATTCATGAATCTTTTATTTTCCTCTTAGAATCATCAG |
| | | | | CTACAAGAAGTTAGATAAAGATGAGCAAATGTTCAAAAATATGCTA |
| | | | | TAACATCATGTGCTTTGTAAGCTTTAAACTTAGAGATAACAAAAATA |
| | | | | CTTAAGAATACATCATGGGTATGTAGCTGAAGATCAAATGGGAAGT |
| | | | | TACATTGCTTCTTTGTGTGTATTGTAAACAATCTGGTATCGTATAAGT |
| | | | | CCAAATTCCATTTTTTGATTTTCAGATAATGTGGTTAGTGGACATCTT |
| | | | | CAGGGCAAAAATTGTCGATATGTCGGAAAGCTTCGTTACTGTAGAG |
| | | | | GTTAGTATGTTTGTGGTTGACATCTTGTGTACCTATCTTCCGTTCAAT |
| | | | | GTAGATTCTTTTTCTTAGTGCTATTTTATTTTCTGTCGTGCAGGTGAC |
| | | | | TGGCGATCCTGGAAAGATGGCTGCTGTCCTGAGAAATTTTAGCAAG |
| | | | | TATGGAATCAAAGAAGTTGCCAGAACAGGAAAGGTCAGTCTTTTAG |
| | | | | ACAATTGCAGTAAATTCACCGGAATACTATGAATGATGCAAAGATT |
| | | | | ACGTGCGGTAAATCGTAAACAGGCTGTTGAATCTCTTCAACTAAATT |
| | | | | ATATCTAATGCGAAGTGCGTTTATTCAACTACCTTTTTTGTGTGAAAT |
| | | | | GATAGATTGCTCTAAGACGAGAAAGGATGGGCGAGACAGCTCCTTT |
| | | | | TTGGAGATTCTCTGCTGCATCTTATCCAGATCTAGAAGAAAAGGCTG |
| | | | | TTGAATCTTTTGTCAGGCCTGCAAAAAGAAGCATCAATGCTGATCCT |
| | | | | GGCTCATCGTCTAGTGTAAGTGAAGTTTTATTTGCTCAACATTTATCT |
| | | | | TATCCTTCTCTTACTTTGTCTTGCATTAGCGTATATTCTTTTTGGTAAT |
| | | | | TACAAATTTAGTTTTTTTTAAATGTTCCTAGGGTGATGTTATTATTGA |
| | | | | CATTGATAAATATTCTTATTGGTAGTATATAGATATATTATATTTCCC |
| | | | | ATAGATTGCTCTAAGACAGGTGGCGTATATCCGCCCGTCCGAAACC |
| | | | | CCGCCAAGTCTCATACGGGCATACGGCCCGTTTGGTAACTGGCAAT |
| | | | | GATAATGAATGTTAGTATAATTTTGGTAAGAATATCTTCTCATAAAC |
| | | | | TTAATGACTTTACTTATTCTCCTTCAACAAGCTCATTTTCCTTACAAAA |
| | | | | TTCTTTATAATGCATCACCACTTAAACCTGTGGTATAAGGTGGTACT |
| | | | | GAAATATTGTGAACAAAAACACTTTTTTATTATTAAAATTTCATTACC |
| | | | | ATAGTGATGACATGATACATATTATGAAGATTTACACTACAAATCAT |
| | | | | TCCCATTACCACCATTTAGAACCATTAGCCTAACGGGCAGTTAATGC |
| | | | | ATTGGGATAATGGAGTGGAAAAAACGTTAATTGTCATTTATGAACT |
| | | | | CAATTTTTTGTTTTGGGTTTGGCACTTTTGCATTTGGAATTTAAAATG |
| | | | | CGTTTATTCAATGAAGGGTGATGTTTATCCAGTGGAGCCTTATGATG |
| | | | | CCTCCATAAATACAGTACTTGATGCTCACTGGGGAGTTCTTTACGAA |
| | | | | GATGATGTAAGTTCTCTTTGTCACTACCTGAATTTTGTTCATCTTGTT |
| | | | | GCACACTTTCATATGATGTGATCTTCAGATCTTTTACTTAATTTACTA |
| | | | | AATGGCTTCATAATTTTCCCAGAATGGCCTAAAATTCGTTAGTTTCG |
| | | | | GTTCATAATTATTTAAGTCATGTCAAACTTTCCTTAAGACCGGCTCTA |
| | | | | TCGAATCTTTAGCCTTATTTGGGAGGTTCTGGTTAATGTCTTTCAATA |
| | | | | TTTGGCTTACTTGTTCATCCTCAGAACTTTCATATTTGTTTGATTTTAA |
| | | | | GCAACGTTATTAGAATTGAGATTCTACAACACTACGATTCTACCTAC |
| | | | | GATCTAGATTGCTCGCTTGTTTCGGATCGTATTATAGTAGAATTGTA |
| | | | | GATCAGAATCATGATTTTAATAACTATGGTTTTAAGAATGGAGTAGT |
| | | | | TTAATAAATCTGTTCTGTTAGTATTCTGATGTACTTTCAACATGCCAG |
| | | | | TCAAGCGGACTTGTGTCACATACTCTGTCCATGCTGGTAAATAATGC |
| | | | | TCCTGGAGTTCTGAACACCGTTACAGGAGTAATTGCTCGTAGAGGT |
| | | | | TATAACATTCAGGTCAGTACGGACTCTGACTGCACCATGAAAGTAT |
| | | | | ATCTCGTCTTTTGCATTATGTTAAGTTGATTGTCCCTTTTCCTTCTTCC |
| | | | | CAGAGTCTTGCTGTGGGCCCTGCTGAAAAGGAGGGTCTTTCTCGTA |
| | | | | TCACAACTGTTGTTCCTGGAAACGACGAGTCAATTGCAAAATTGGTT |
| | | | | CAGCAATTAAACAAATTAGTAGACCTTCATGAGGTGAGCATCTATTT |
| | | | | GCGTTCAACGTAGACATAATCGTCCAAATTAGTTAGTGTCATGTTTA |
| | | | | CTTATGTGTTAATATTATTTTGATTTTTGTTTTTTTGACTATTAACATG |
| | | | | CAAAACAGTACTTTACCTCTCTACTAACGTGTGCCGAGGCCCAAGGT |
| | | | | TTCTACCTAGGATCGATGAGAGGTTAAGATCGAAATCAATGCAACC |
| | | | | GGATCGTAGAATTGTATACATTAATTTGCTCAGCGTAACTGATCATC |
| | | | | AATTATATTTGTTATTTTTAGTGTTTTGAGGCGTAAGCCGTAAATCA |
| | | | | AAATTATTTGACTTCATCTATTAAGTTGTGAAAAATAACAATTTTAAT |
| | | | | AAGCATTGAAACTGCAAATCAAGAAAAATATGAATTTTATAGTCATA |
| | | | | TTGTTATAATAATTTTACATATGTTTATATATATTCGAATTCTAGATTT |
| | | | | TAAGATAAATTTTTTACGATTGAGACTACTCACATAGGAACTGATCG |
| | | | | TTGAATTGTAGGATCGTGAATCGTGTTATGATCTAACCCCCTAAATT |
| | | | | CTTGAGATAAGTGTGATGATACGATCCTACCAACTATAAGATCCCAC |
| | | | | CTACAATCCGGATTGATTGCCAATTTTTGGATCGTGGATTGGGATTG |
| | | | | GGATTCTGATAACCATGTCGAGGCCTAAAGAGAACAAAAGAGATG |
| | | | | AAAGGAGTTAACACCAATTCTTTAGCTTTGTGCTTGTATCAACAAAA |
| | | | | CACCATGCGAGATTCTTGTTATTCTAAAAGCCCTATTCAGCCCGTAC |
| | | | | CTATCATTTGTTTTATATCGAATCAGTGAAGCTAAGTCGAGTAACAT |
| | | | | TTTTCGTGTAATCTTTAATTGCGTAGATAATTCAGATATCTTGCACGC |
| | | | | TCTTCAATTCTCTACTATGGAGTTCTGATTGTCTTGTAGTTTGTATAT |
| | | | | AAGTACTACAATTATGATTGAAAGATTAGAACATCTTATTCCGGAAA |
| | | | | GGTTGTTGTCGCAGATTCAGGACCTTACGCACCAGCCATTTGCAGA |
| | | | | GCGAGAGCTTATGTTAATTAAAGTAGCGGCAAATACTTCGGCCAGG |
| | | | | AGAGACGTCCTTGATATTGCTAATATATTCCGTGCAAAACCTGTGGA |
| | | | | TGTTTCTGATCACACAATAACATTACAAGTAAGCATAAATGAATTTG |
| | | | | GTAGTTTATTTAGTCTTCTCTTGTTACTCTCAACAATGTCTATTCACA |
| | | | | AATTCTTGTGTGAGACGGTCTCACCGTGATACGTTCCCCATACATGG |
| | | | | ATTGAATAGCCCAACTAATACAATTATCAGCATATGAGCTTCAATTA |
| | | | | GTCTCTTACAAGAGTAAATCATGTCTATATCCTTATTTCGATGGTCCA |
| | | | | TTTCGAATTGAAACGAATATAAATAAAAAGCTAATGGAATGCGAGT |
| | | | | ATTTCACTAATAACGAAAACCGTAGGGAGCTAAGACAGTTTACGGT |
| | | | | ATTGAGGATCTCTGTAATTTGTCTTTGTTTTTTTCAGCTTGCCGGTGA |
| | | | | TTTAGACAAAATGGTTGCGCTACAGAGATTATTGGAGCCTTACGGC |
| | | | | ATCTGTGAGGTATGTTTATATGCATATTTATTTCTCCCGAAACCGATT |
| | | | | AGAATCATTTCATGTGTTCGGGTCAAATAGGAAATATATGGCCATTT |
| | | | | GTAGCTTTTCAATAGACTCCGAGAAGGCGAGAACCCTTATCTCCTTA |
| | | | | ACTACTAAAACATTTACAGCTCGTTGATTGTCGGTAATAAAAGGTGA |
| | | | | TATTGAGAATAATTTGTAGTGTAAATTTTTCATGATTGTATTATATCA |
| | | | | TTCCCTGGGTAATGAATAGTTAATCATAAAAAAACTTTTTTTTTCGCA |
| | | | | ATTTTTCATTACCATTTGTGTCACCTCTACGAATGGTAATGCATATGA |
| | | | | TTGATTGAATGAGCTTTATGAAGGAAATAAGATGATTGAAGTAAAT |
| | | | | CAATCAAGATCGTTAAACTTGTAAAGAGATTTTCTCACCAAACTTAC |
| | | | | TCTAAATTTCATTTCCATTTCCACTATTCAATACCGATAACTAAACGG |
| | | | | GCATCAGCACCATTTAGTCGAGTACTTGATTTCAATTCAATTTAATTC |
| | | | | AGTTCAGTTTAGTTGTAAGTAGTTCTGGAGATAGCGAACTTCAATG |
| | | | | ATCATGGACTGACTGTAACATTTTATATCTGTCTTCAAGGTGGCACG |
| | | | | GACTGGAAGAGTAGCACTAGCTCGAGAGTCTAGGGTAGATTCCAA |
| | | | | GTATTTACGAGGATACACTCTTCCGTTGTATGAATGAAAAACTCCGG |
| | | | | CTATGTCTTTCCGACATTACCTTGTCCGATTCCTTCCGCTCTTCTACAC |
| | | | | TGCATTACGGGCAGAACAATTGCCCACGAGTAGAGTAAATTAAAAG |
| | | | | GGAGAAGCAACAATACCGATCTTTGCTTTGAAGATTGTATAACAAT |
| | | | | AGAGTTTGTATTTGATTAGTATTTATTTGCTCAACGTTGTAATTACAA |
| | | | | TTCATCTTGAGGTTTTGATTTAGCATTGTACCCGGGCTAAGATAACC |
| | | | | TAGTTGTTTCAAAAAAAAAAAAAAAA |
| 1705 | Amaranthus | gDNA | 5396 | AATCTGGTATATTTTTCATTGATTTGTGTGTGTTGTTGCTTCTTGCAT |
| | palmeri | Contig | | GTTATTTGTTCTTCCGTTTGATTTTTATACAAGAGAGCGTGCAAATTT |
| | | | | TAGCATGTTGTCAGTTCTTAAATTGATCACTGTCTGTATTTTGTTAGT |
| | | | | CTACCCCATTAAAACGACTCACTAAATCACTTTTTTTATTTGGGTGAA |
| | | | | GATGTTTATGATCTTTGCTACAAAGATTTAATCGGAAATAATTTCTA |
| | | | | GTTATTATCACTAATAAGGGTAAGATCGTATACATTAGACTCCAAAC |
| | | | | CCCGGCCGTAATAGAATTGGGGTAATGATTAAGGTAATGAAATGTT |
| | | | | GTTGCCTGTATTTTTGTAATGAATTCCAAACAGGCTGTTAAATATAG |
| | | | | ATACTTTCCCTGTAAAAATACAAGAGTTTATAAGGCTCTTATTCAAC |
| | | | | CCAACATAAGTTTATTGAAAGATTTATTAGTAGTCTTTTTGTGATTCG |
| | | | | TAACCTTGTAATTACCAAGTACAGCTCTTTATTTCTATGACATTGAAA |
| | | | | CCGTCTGCAATCAAATTGACCTAGTGCTAAAAGCTGCTTGATTTGCT |
| | | | | TATTGCCTTAAGTGAAAATTTATAGCAAGCGTTTTGGAGGAAGAAA |
| | | | | AGCAAAAAGATCGATATGCTTTCATGTCTTCAGAAAATTAGTTCCGA |
| | | | | AAGTTGTGGAGTATAATAGAGTTCAACTATTGTGCATGTAAAATATT |
| | | | | AATCTCAAGTAATTCTGATAGAAGACCATCTGGGAAGATTTAATTA |
| | | | | GTGCTAATCTGGGAGGAATTATTAAGATTAGTCAGTAATCTGGCAA |
| | | | | CATTTATCTAGGAGGATAGAAAAGGCATTGACCGTATGTAGTAATA |
| | | | | TTAGTGCATGGGAAGTTCACGTGCAGTTGGAAGACACTTTTCCGTT |
| | | | | ATAAAGGGTCATTATGAAATAGTGGACACGTGTTAGTAGGAGAAG |
| | | | | CTAAGAGCGCCATAGAAAATAAAGGAAAAACCATTAAAGTAATTAA |
| | | | | GGGCTTGAAACAACCAAAACAAGCACAGTGGCCTGTGGAGAATTTT |
| | | | | GGGGATTAATTTCTTTCTCTTCCAACATTATAATGTCAATTTATGTCA |
| | | | | TTTGTGCTAAAGTGACACTTAATGTACCCTATCTTCCTTCGACTTAAT |
| | | | | TTAAACTATCGTGACTTCAACTCTTATCTTTATTGTTATAACCTAGCTT |
| | | | | CTACCCATTACGCGAGTTCATTTTCATCTACCTTTATATAGCTTATCA |
| | | | | TTAATATGTTCTCATAATAATTCAAAAGTTTGCATCTATCTAATTTTA |
| | | | | GAGCTACAAGTGTTGCTTGTAGTAATTGTGTGTCGTTAGATTGTAGT |
| | | | | GCGTGAACTATCCATTTTTCGTGAACTGTTGCATTTCTTGAATGACTT |
| | | | | GTCTCTAGAATGATGCTCCTTAAACTTACTTCTTTTGTATCATGTATG |
| | | | | TTTAAGGGAGAGACGACATACAATATCAGTATTTGTGGGGGATGAA |
| | | | | AGTGGAATGATTAATCGAATAGCAGGGGTTTTTGCCAGAAGAGGTT |
| | | | | ATAATATCGAATCACTCGCTGTTGGATTGAACAAGGACAAAGCTCT |
| | | | | CTTCACTATTGTAGTTTCTGGAACGGATAATGTGTTGCAGCAAGTGA |
| | | | | TGGAACAGCTTCAAAAGCTTGTCAATGTTTTGAAGGTTATTTTCCTTT |
| | | | | TGAATAATTACCGTGGCATCCATGTCTTCAATTGTTTAGAATTCTCAT |
| | | | | CACTAAGCACAACTTGTACATTTAGGTTGAAGATATATCCAAGGAG |
| | | | | CCTCAAGTAGAACGTGAATTGATGCTTGTAAAAGTTGGAGCTGATC |
| | | | | GGAATAACCGTGCTGAGGTACCTAATAATATATGTCAAAATACCCA |
| | | | | ACTGTACAAAAGAGTTGATCTTATTAGATACAGGCAGATATGTTAG |
| | | | | CTACAGCCTACATCTCTATGTTTTTTGTGGCAACTTTCTGCTACATGC |
| | | | | CGATCTTTCTTAGTTTTCTCCTAGTGAATTGTAATTTTTAATATATAAT |
| | | | | TTGTTATTCCACTATGTCTGTTCCTGATTTACTTTCGCTGATCACATC |
| | | | | GTTCGTGTTTCTTCTATTGTAGCTGATGTGGTTGGTGGACATCTTTC |
| | | | | GTGCCAAAATTGTGGACATATCGGAAGAGTATCTTTCAATAGAGGT |
| | | | | AATCACAGGATATTTCATAAAATCAATGTTAATTTTGGGAATATTGG |
| | | | | GAATATTGTGACAAGTATCAATGTTTTATGTACAGGTTAAGTGTTTA |
| | | | | ATTTGCATTTTCCACAAGCTACTTCATCCTCTTCTCCGAAGATGTGTA |
| | | | | AAATTGTAATATACATATTCACACCAATTTTAAAATTTTGGAAGAGC |
| | | | | GACATACAAATGATTTATGACCTAATTTTGCTGTATATATACATGCTT |
| | | | | ATATTTTCTTCAAACAGCTTTAGAAAATTGATTATATTTTTCCTCCTTC |
| | | | | AAAGAAAACAAGTTTTGTGAGATAAAAGCTACATGTTTAGTAAGCA |
| | | | | AATTTAATGAAGACTGATGAAAACAAAATTTGTACTTTTGAATGGTT |
| | | | | TTATATATTTTATGATCTTTGTAAGCTAATATGGAAACTATAAGATTG |
| | | | | TAGTTGGTTTTCTTTAGCTCTTTTCGCTTTTACCTTTCATTTTTTTTTAC |
| | | | | ATGCTTAAAAAAGCGACTCACCTAGTACTTTGATATCCAGGTCACTG |
| | | | | GAGATCCAGGAAAGATGGTTGCTGTCCTTAGAAACCTAAGCAAGTT |
| | | | | TGGCATCAAAGAAATTGCTCGTACCGGAAAGGTTTACTAATTTTTTT |
| | | | | TGTAAATTTTTCATTGTTGTTTAAGAGATCTTTTAGTTTCCACATTGC |
| | | | | ATAATAAATGTGAATGCCTTTTGTTTATTGCTTGTCCAGGGTTTTTAT |
| | | | | ATTTTTTTAAGATGTAAAGTTTAATATCTAACAGATTGCTCTAAGAA |
| | | | | GGGAAAAATTGGGCGAGTCTGCTCCTTTCTGGCGTTTTTCTGCTGCT |
| | | | | TCTTATCCTGATCTTGAAGAAGCTATCCCTATGGATTCTCTTTCTGGA |
| | | | | GTTGCAAAAGCAGCAACTGCTGCTGGATCATCGGATTCGTCTGTGG |
| | | | | AGGTGTGTTTCTTATTTGTCCAAAATTAAGTGTCCAAGTGTCGTGCC |
| | | | | CAAGTCCGAATATCGAGGATCAGACATGGGTACTTGAGTTAAAATG |
| | | | | AAGAGTCCGGGTAACAAATGTAATTGATTTAAAAACATATAGTTGC |
| | | | | CGGATTTTAAAATTTTTTTAAAGGTAGTTGGAAAATTTTCCTTTTATT |
| | | | | GGAAAAACTTTTGGAGGACAATATTTTTGTGAATGTGTCTCGAGTG |
| | | | | CCTCTTATGTTAAAGGGCCTCAAGACGAATAGGAAATGGCGGGCG |
| | | | | GGTCGAATGCGGGTGTGCAGGCTGGTTGCTGGCAATGTTTCCGCA |
| | | | | GGCTGTTGTTGGTGTGCTTTTCACTTCAGTAGCAATTTACAGCAAAT |
| | | | | TAGTAGCAAATTTACAGCAAATTTACAGCAGAATGTTAGCTCAATAA |
| | | | | CCCATCATGACATAAATATTAATGTATAACATAACTTTAACTACAAA |
| | | | | ATACAATTTAGAATCATCAAATCACAATTTAGTTGTTTAATATACATT |
| | | | | AAAATATTTCTTTGTAGGAACTTCAATTCTATCTCTTAGTAGCATAAA |
| | | | | TTTTACAATCAAACGCAACAAAAGTATTAAGTTAGAAAGGGAGTAC |
| | | | | TGCATGTGGATTGTCTCAGCTAAAGTGCTGCAGCAGCTTTGCACATT |
| | | | | ATCTAGTGAGACATTGGAAATGATTAGGAGCTTTGGGATAAATATG |
| | | | | TACAGAAACACTTCTCTTCTTGCCATCCCAAGGATACTGCAGCTTCA |
| | | | | GGAAAATGAAGGTCCTGTATGATTTTTAGAAACTCCACCAAATTCTG |
| | | | | AAGATTAAGCTCACCTCCGTACCTAACATTGAGATATCTTCAGTATA |
| | | | | TTCGCACCTACCTGATACCTTGCTATGAGATCTCTGTTAATCAGCTTC |
| | | | | ACTAAGACAAGCCAAAATTTCTAACTAGAAGTCATTTTCTGCAAATT |
| | | | | TTAATGCAGATTCCAGAACTACCCATTATCATTAAACTTGAATATTTC |
| | | | | CCCTTTTTATAACATTTCTGATGCCTCTTGTATGCTAATCTTTTCTAAC |
| | | | | TATATACACTACCACAAAACACAAATCTATACTTACTCTAAATAAGTT |
| | | | | TAGTATGAATTACATTTTATTTTTAGTGACTACGACTTAGAAGTATAT |
| | | | | CTTAGACAACTTACATTGTAGATTAATATGACATAATTTGTTTCTTCA |
| | | | | AAATAAAAAATGACATAATATGTTATCAATCTAGACCTCCTCAAGAC |
| | | | | AAGTATGATAAAAAAAAACTCCAATGCAACAAAAATTAATTGATTTT |
| | | | | TTTGAAAAAAGATAGAAGAAAGTGGAAGAGGCAACACAAGTAAAG |
| | | | | AAGGCTTCTCGATCCTCTGAGCACTCTAAACCTTGCAATACAGTAAA |
| | | | | CTATAAATAAAAACAACAAAGTTCAGATATCATCTCAATATTTTCCC |
| | | | | CAAAAAAGGTTCAACATCATCTTCTTCCTCTTCTTCCACAATCTCATT |
| | | | | GTCATGTTCATCTAAAAGCCTATCCTTGTCCTCAATGGTGTTGTGCA |
| | | | | TAGCACGCCTTGGAGGCACACCAACACTTGCACTGCCTTTGACTTCA |
| | | | | AGAACTTTGAGTAGCAACAATCGTCTTATCACCTTGGCCAAAGGGA |
| | | | | GATTTGGAAGTCATAATACTCACTGGAATGTATATAAAATGACTATA |
| | | | | ATATTAAAAGACAATGAAAATGATTTTTGTGCATTTAAAAAGATGTT |
| | | | | TTCAAGTGACTATTTTTGGGTTTATCTTTTCTATTTCTTCTCTCTCTTT |
| | | | | ACTTGGACCCTTTTCTTCCTATTGCAACTTGGATCTTTCAGATTGGAC |
| | | | | ACTCTCTTTGTGGCTATATGATGTTTTCTAAGCTCTATAAGTATCTTT |
| | | | | GCTCTACAACTTTCTTGCCTTATTCTTCTATTACCATCTCCCACATACT |
| | | | | CCCAATATTATTACTTAAAGAATTTTGGCTGCTTGTACAAGTATGTTT |
| | | | | GGTCCTTGAACTACATTCTGTCTCCCCCCTTCCATTGTATTTTCTATTA |
| | | | | CGGGTTTTCTGAAAATGTCCTCCCTGCCCTTATTTGTCATTTATGGCT |
| | | | | ACATGACTTGATCATTGCACTATATGATGGAAATTTAAGCAGTATTG |
| | | | | TTGTAGTTGTACTTTTTTGATGAATCATCATGTCTGCTTCTGAATTGG |
| | | | | ACTCACTTCAATATCTTGAAGGTTTTTTTCCTCTATTTTTTAGGGTGA |
| | | | | TGTTTATCCTGTGGAGCCGTTTGATGGTTTCTCCCCTCCAATCTTAGA |
| | | | | TGCTCATTGGGGTATTTTGAATGAAGAAGATGTAAGCGACTTTCCTT |
| | | | | TCTCTTTTATTTATCTCGGGTTGTTATTTTTTAGTTCCATTGTTTGCCT |
| | | | | TTCGTGTTCATTTTCGATATAAATAGTTTTATCATGCTTCAGTTTCAA |
| | | | | ATTTTAATGGATA |
| 1706 | Amaranthus | gDNA | 5223 | ATAATATTATCTTTTTGGAATAATATAATTAAAAGCAACAAACAAAA |
| | palmeri | Contig | | TGTATAAATTTAACTAATATACTTAATTCAAAAAAAAAAATAAATTC |
| | | | | AATTAATATACCAGTAAGCAGTAAGTGTGTTTCGCATTTCAGTATTT |
| | | | | GAATAATATTATCTTTTTGGAATGATTATAATTAAAAGCAACAAAAG |
| | | | | TTATAATTTTAATTAATATATATACAAGTAAGTGTGTTTTGCATTTCA |
| | | | | ATATTTCAATAAAAAATTATAAGTGAGTTGTCTATTTATAATAAAAT |
| | | | | AAATAATGCACTACTCATATTCTTCTTCAAAAAAATCCTCACTACCAT |
| | | | | CTCCATTGTCCCCATTTCATTTCTTTCTCATATCTCTCCAATGGCGGCC |
| | | | | ATTTCCTTTAACATTAATGGCGGAAAGATTGGAACTTTATGTCCAAA |
| | | | | ACCTAAATATGGTTGTGGGTTTTTGAGAAAATGGGATTTTGGAGCT |
| | | | | CATTCAACTGTATCTACAAAACCCATGTCAAAAATTTTAAGCTTGAA |
| | | | | AGCAGTTGAAGTTTCTGCTGATGCTACAGTAAATGCAGTTTCTGTTT |
| | | | | CAGCTAATTCTAGGTAATGTACTTAATTAATATTACCATTTTTCATTG |
| | | | | AATCTAAACTTTTTTTCTTCATGTGGAATTGTGGATATAGGAGTATG |
| | | | | TATGGAAAGAAATATAAGGGTTTTTGCTTCTTTATTGGTGTTTGTTT |
| | | | | AAAAAATTGTATGGTTGCATACTTTTGTGATTATTCATTTATTCTGAG |
| | | | | CATGTTTAACATGTTCGATTGTACAACCTCTAAAAAATTGAATGCAA |
| | | | | AAGATACAAAATTGTTAGAAAATATCATAATTTTTAAAATTTCATGG |
| | | | | GGTCTTAAAATAATTGAAATTTTCAATTTTTGCTCCGGCCCTGATCCT |
| | | | | CCTAAAGCTCACTCTATAAGGAGTCACCAAAAAGTAAAGTTGAGTA |
| | | | | TAATAGTCATGTGACTCTAATTTTAGCCTAGGTGGAAGAAATAGGG |
| | | | | TCAAGGTCAATGGAATGTTGTTTGTTTGTTTGTTTGTTCTGGTATCAT |
| | | | | TACAATTTACAGATATGAAGTTAATAAGGTTTTGGATTATATTGTAC |
| | | | | CATATTAAATTGGCTTATTAGACTATGTGTACTTTTATTTATGTGAAA |
| | | | | TTGAGATGATGTTTGCTGCCAAACGCCAATCGAAAACAGATATATA |
| | | | | CTATCATTAACAAGGGTAAATGTGAGGTTATTTGATGGCATTGAGG |
| | | | | TGATATAACATTGTATGAAGTTGCAAATTATGAAGCTTAATCTTGTA |
| | | | | TTAAGTGGGAGATAGAGCATCTTTTGGTTGTGATGTTATAACTTTAG |
| | | | | TTGGATTTTGTAAGTGTTGTTGCAAGTTATTCATGAATTAAGGAGAT |
| | | | | GTGCCTTTCTATGTGAATCACATATGTAAGAAGTAGTTTATGTGTTT |
| | | | | GTTGATCTTTATACTGTATGATGGCCGATTCAAATGTGGAAGTTGAT |
| | | | | ATGATCCTTTTAAGGTCTACTGAGAAGTTGTTCTTCAATTCATTATAT |
| | | | | TATCCTTGGTTTTCGACCATAGGAGGTCACATGTTCGAATCTAGCCT |
| | | | | CATATATAACATATCTTGAGGCTTCAACCATCAACTTAAACTTTGATT |
| | | | | GAGTTGCTTCCCTGTGACTTTCTCGCCTTCGCTTCACCTTTATGCTTG |
| | | | | GTTTCAGCCTTGCTAAGTTGCTAGTCGTGGAGAGGGTTACTTCAAG |
| | | | | AAGGGTGTCAGCATATATCACTGCAGATTTTTTTGAATACAAAATCG |
| | | | | TTTTTAAAGAAATCATACATGGTTATCTTACTGCTCCTTGATTCGATG |
| | | | | GAGCAAAGTTTTGTTGTTATTTGATTTCCGATTATAATGGATCATTG |
| | | | | TAAAACGACTCTTTAAATTCTGCGCACTTCTTATTTATGTAAAGTTGC |
| | | | | AAATGATGTTTGCTATATATGTGGTTATCTTATTGTATCTTGTTATTT |
| | | | | ATTCTCAGGTAATGAAACAGAATTCAAACTCTAATGACATTTCATAT |
| | | | | ATTTCATTTTGTGTATACAGGGTGATGCGTCACACAATTTCTGTCTTT |
| | | | | GTCGGGGATGAAAGTGGGATAATCAATAGGATTGCAGGTGTTATTT |
| | | | | CTAGAAGAGGATACAATATCGAGTCTTTGGCTGTTTGTTTAAACAA |
| | | | | GGATAAGGCTCTTTTTACTATAGAAGTGTGTGGAACTGACAAGGTG |
| | | | | TTGCGCCAAGTCGTGGAACAGCTTAACAAGCTTGTTAGTGTTTTGAA |
| | | | | GGTAGTTAATTTCCTTTGTTCTTCCTTTTATTGGTAAATGGTGATAGT |
| | | | | GATACCCTTGTAACATAAATCTTCGAATAATGAGACTATGACATAGT |
| | | | | GCTTGGTTCACCACTTTACGGATTGCGTTCGATAAAATTACTTTATG |
| | | | | AGGTCTAATGAACCCGTTTTCCAAACTAGCTTATACAAACTAAACAT |
| | | | | ATCCTGAATTTGTTTTTTACTAAAATAAAAAAAAATTCTTTTAATATG |
| | | | | GAGCGTTACGTATCAATCAATATAATGGATCATCTTATATGCATATG |
| | | | | TTTGTACAATGTACATTTTTTGTTAGATTGTCTATTTGTCCTGATAAC |
| | | | | AACCTTGGATGATTCTTGTATTTCTTGTAGGTTGAAGATCTATCGAG |
| | | | | AGAGCCACAAGTGGAACGTGAACTGATGCTTGTAAAGCTTAATTCT |
| | | | | GATGCAAATTCCCACGCAGAGGTAGTTCCAAAAATTCAATGATAAT |
| | | | | TTATGAATGGTATATTTTCCACTTTTTAAGTTCTAATTTCCTCGGCTG |
| | | | | TTGTCAGTTAGAGTCTAGCAAGGAAAAAGATAGAGATGTTTTGCTT |
| | | | | TAGATTTTATCGATGATCGAGAAAGATGCTATACTTCATATAAGGCC |
| | | | | ATGACTGTTACAACAACAACGCCAGAGCTTTAGTCCCAAAAGTTCAT |
| | | | | GGCTGTTAGATAGTTTAAATTTAACTAGAAGTACATCGTGTTGTGTG |
| | | | | AAGCTCGTATAAGAAATTGCATTAGTGTCTTTGTGAGAAATCTAATA |
| | | | | CCGATTAACGTAAATTTTTAATTTTTGATCTTCAGCTAATGTGGCTAG |
| | | | | TGGACATCTTCAGGGCAAAAATTGTGGATATCTCAGAAAGCTTGGT |
| | | | | CACTGTTGAGGTTAGTGGTATATTTGTTTGACTTCTCGCGTATCTCTT |
| | | | | CTATGTTTGCTGAATTAAGTCCGATTTTTTTGGATTTGCTTCCATGAT |
| | | | | AAATGCAAGAAATTTACCTACAAGAAGTTGACCATTTTCCTCCGGTC |
| | | | | GATGTAAAATCTTTTTCCTTATTGGCATTTTCTTTGGTGCTGTGCAGG |
| | | | | TGACCGGAGATCCTGGAAAGTTGGCTGCTGTCCTAAGAAATTTTAG |
| | | | | AAAGTTTGGAATCAAAGAAATTGCAAGGACCGGAAAGGTTAGTCTT |
| | | | | TTAGACATTTGCAGTAAATTCAGCATAGAATTCGGAAAGCCGCAAA |
| | | | | GAGCACATGTCAACTATTGTGGCTAGCCAGCCAACTATTATGCATTT |
| | | | | CTGGTGAGATCAATGTGAAACAAGTTTAGCTAACTACTTGTTTTGTT |
| | | | | TTATACTTAATATGATAGATTGCTCTAAGGCGAGAAAAGATGGGTC |
| | | | | AGACGGCTCCTTTTTGGAGATTCTCTGCTGCTTCTTATCCAGATCTAC |
| | | | | AAGAAAAGGCTGTTGATGCTCTGGCCAAGCCTACGAAACGGAGTAT |
| | | | | CAATGGTGATTCTGGCTCATCTTCAAGTGTAAGTGATATTTTATCTTC |
| | | | | TCGACAATTATCTTTTTCCTTTTCTTACCTTGCATTGCCTATGTATACT |
| | | | | TCGTTCACCTTGTATTGCCTATAGACTTAAACATGCTTTTATTTTTGA |
| | | | | AGGGTGATGTTTATCCGGTGGAACCTTATGATGGTTCGATGGTTAA |
| | | | | TCAAGTGCTTGATGCTCACTGGGGCGTACTTTATGATGGTGATGTA |
| | | | | AGTGCTCTATCACTACCTGAATTTTTTGTCACTAGGTTGTCCAACGG |
| | | | | TCCATGTGATGTCATGTTCAAATCGCTTAGCCCAGTGTTTTCTAATAT |
| | | | | AGCTCCAAATTAATTGTCTGGATTCACGATTCACCCAAAATGGCCTA |
| | | | | AATTTTTTCGTTTTAATTTACAATTGTCCAAGTCATGTCAAACATGTA |
| | | | | TTAAGGTCTAAACTCTGCTCTGTTACTTGGACTCGGGTACTGAGTCG |
| | | | | GCAACGTGTCCAAGTGTCGTATGCGTCTAAATATTCAATTTTACGCT |
| | | | | TAAAATGAAGTATCTAAGTGTCATACCAATGTCCGGGCATCAAGGA |
| | | | | ACGGACATAGGTACGTGAAGCAAAATGAAGAGTCCGAGTAATAGC |
| | | | | GTTTCAGCCTCCTTTGAATTTTGGAGGGTTAAAGTTTATGTTTTTGG |
| | | | | GTTTTCAGATGTTCGTCTTTCTGTTATTCATGTTTAATTTTAAGAATG |
| | | | | AAAGGTAGTTCGACTTTTCAGTTTTTTGAGCTGCACAGTACTATAAA |
| | | | | TTGGTTTTAGTTTGCTCTGCTAGTATCATGAATTGCTTCGTTCTTCAT |
| | | | | GCCAGTCAAGTGGTCTCCGGTCACATACGCTGTCCATGCTGGTAAA |
| | | | | TAATGTTCCCGGAGTTCTTAACACTGTTACAGGAGTAATTTCTCGTA |
| | | | | GGGGTTATAACATTCAGGTCAGTGTGGACTCTGACTACAGCAAGAA |
| | | | | AGTATATAAAGCCGTTTCTGTTGTGTAATGACAATCGTCCCCTTTTCA |
| | | | | TCCTCTTCAGAGTCTTGCTGTAGGCCCTGCTGAAAAGGAGGGTCTTT |
| | | | | CTCGTATCACAACTGTCATTCCTGGAAACGACGAGTCGATTGGAAA |
| | | | | ATTGGTTCAGCAATTTAACAAGTTAGTAGACCTTCATGAGGTGAGC |
| | | | | AACTATTTATGTACAAAACCGAATAGTTGTAATAAGTGGTATAGACT |
| | | | | ACATAGTAATAACTTTATGGTTGTAAGATGCAAAGTACCCTTATCAG |
| | | | | TTATCACTATAATATGTTCTAAGAAGCTCAAAACTGAAGTGGAAGA |
| | | | | GAAAATTGTGACATTGGTTAGCTCCGCGTTTATGACAAACTACTTGA |
| | | | | TTAATTCTTGTTTTTTTGAATGCTCAGTTCAATCTCCTTTGTAATATGT |
| | | | | TGATGCTCGTTAAGTTACCTATTTTCTTTGTAATACGTAAACCGTGAA |
| | | | | GTTGTGTTATCTTAGTCCAACGTCACATCGTGCTGCTCGAGTCATTT |
| | | | | GATTTAAAACGCTATTTATATTGAATAGTGTAACATATAACACAGAA |
| | | | | AAATAGTAGTCGGGTTGGTTCAACACACACAATAATTTCCCACATTC |
| | | | | TCCCTTGAATGAAAGATACGAGAATCTAACATCAGAATGGTTGC |
| 1707 | Amaranthus | gDNA | 4591 | AAATTAGTTGGTTAAAAAAACTTTTTGCTAAAACAAAATGTACTTTT |
| | palmeri | Contig | | CTAATACTCCTATCTAAAACCCCAACTAATACTAGTGTGTCTTCCTTT |
| | | | | GCACCCCGGTTGTCCTCGTCAGGCCGAGTTAGCTGTGTTTCAAATTT |
| | | | | CCATCCATGGAGGCTGTGTCGACTCACCTTTCAACGAGTTTTAACTC |
| | | | | CATTCCGAAAAGCAATAGACTGAACCACCAAACTGCAAAACGATTA |
| | | | | GGGTTCTCCTTGAAACCCCATTCGCTAGGTTTTAAGTTTAACTCCAAT |
| | | | | AGTGACAGGAATTCGGAGTTTGATAAACTGGTTGTATCTGCAAGCA |
| | | | | ATGTTGATCAACTGGGAAATCAAAGTAACTTATCCTTTAATCCCCCT |
| | | | | TCTTCCTCTCGATCAAAGTACGTTATTTTTCCCTTTTTTGATTAATTTT |
| | | | | CACAATGTATAATTTAGGTTTGGCTTGTTGTTTTATCTTCTACTAGTT |
| | | | | AATTTTGCTTTTCATTAATTATTGAATTTTGAAATGAAAATGAGTGAT |
| | | | | TATTTGATTTATGAGCAATTGTTGTCACTCGTGCCAGCCTGAAGGAT |
| | | | | TGGGAAAATTAGTCAATTATGAATCTGGTATATTTTTCATTGATTTAT |
| | | | | GTGTTCTGTTGCTTCTTGCATGTAATTTGTTCTTCCGTTTGATTTTTAT |
| | | | | ACAAGACAGCGTGCAAATTTTAGCATGTTTTCAGTTCTGAAATTGAT |
| | | | | CACAGCCTGTATTTTTTTAGTCTAAATTATAACGACTCATTAAATCAT |
| | | | | TTTTTTTATTTGGTGAAGATGTTTATGATCTTTGCTACAAAGATTTAA |
| | | | | TCGGAACTAATTTCTAGTTATTATCACTAATAAGGGTAAGATCGTAT |
| | | | | ACATTAGACTCCAAACCCCGGCCGTAATAGAATTGGGGTAATGATT |
| | | | | AAGGTAATGAAATGTTGTTGCCTGTATTTTTGTAATGAATTCCAAAC |
| | | | | AGGCTGTTAAATATAGATACTTTCCCTGTAAAAATACAAGAGTTTAT |
| | | | | AAGGCTCTTATTCAACCCAACATAAGTTTATTGAAAGATTTATTAGT |
| | | | | AGTCTTTTTGTGATTCGTAACCTTGTAATTACCAAGTACAGCTCTTTA |
| | | | | TTTCTATGACATTGAAACCGTCTGCAATCAAATTGACCTAGTGCTAA |
| | | | | AAGCTGCTTGATTTGCTTATTGCCTTAAGTGAAAATTTATAGCAAGT |
| | | | | GTTTTGGAGGAAGAAAAGAAAAAGATCGATATGCTTTCATGTCTTC |
| | | | | AGAAAACTAGTTCCGAAAGTTGTGGAGTACAATAGAGTTCAACTAT |
| | | | | TGCGCATGTAAAATATTAATCTCAAGTAATTCTGATAGAAGACCATC |
| | | | | TGGGAAGATTTAATTAGTGCTAATCTGGGAGGAATTATTAAGATTA |
| | | | | GTCAGTAATCTGGCAACATTTATCTAGGAGGATAGAAAAGGCATTG |
| | | | | ACCGTATGTAGTAATATTAGTGCATGGGAAGTTCACGTGCAGTTGG |
| | | | | AAGACACTTTTCCGTTATAAAGGGTCATTATGAAATAGTGGACACG |
| | | | | TGTTAGTAGGAGAAGCTAAGAGCGCCATAGAAAATAAAGGAAAAA |
| | | | | CCATTAAAGTAATTAAGGGCTGAAACGTCCAAAACAAGCACAGTGG |
| | | | | CCTGTGGAGAATTTTGGGGATTAATTTCTTTCTCTTCCAACATTATAA |
| | | | | TGTCAATCTATGTCATTTGTGCTAAAGTGACACTTAATGTACCCTATC |
| | | | | TTCCTTCGACTTAATTTAAACTATCGTGACTTCAACTCTTATCTTTATT |
| | | | | GTTATAACCTAGCTTCTACCCATTACGCGAGTTCACTTTCATCTACCT |
| | | | | TTATATAGCTTATCATTAATATGTTCTCATAATAATTCAAAAGTTTGC |
| | | | | ATCTATCTAATTTTAGAGCTACAAGTGTTGTTTGTAGTAATTGTGTG |
| | | | | TCGTTAGATTGTAGTGCGTGAACTATCTATTTTTCGTGAACTGTTGC |
| | | | | ATTTCTTGAATGACTTGTCTCTAGAATGATGCTCCTTAAACTTACTTC |
| | | | | TTTTGTATCATGTATGTTTAAGGGAGAGACGACATACAATATCAGTA |
| | | | | TTTGTGGGGGATGAAAGTGGAATGATTAATCGAATAGCAGGGGTT |
| | | | | TTTGCCAGAAGAGGTTATAATATCGAATCACTCGCTGTTGGATTGAA |
| | | | | CAAGGACAAAGCTCTCTTCACTATTGTAGTTTCCGGAACGGATAATG |
| | | | | TGTTGCAGCAAGTGATGGAACAGCTTCAAAAGCTTGTCAATGTTTT |
| | | | | GAAGGTTATTTTTCTTTTGAATAATTACCGTGGCATCCATGTCTTCAA |
| | | | | TTGTTTAGAATTCTCATCACTAAGCACAACTTGTACATTTAGGTTGA |
| | | | | AGATATATCCAAGGAGCCTCAAGTAGAACGTGAATTGATGCTTGTA |
| | | | | AAAGTTGGAGCTGATCGGAATAACCGTGCTGAGGTACCTAATAATA |
| | | | | TATGTCAAAATACCCAACTGTACAAAAGAGTTGATCTTATTAGATAC |
| | | | | AGGCAGATATATTACCTACAGCCTACATCTCTATGTTTTTTGTGGCA |
| | | | | ACTTTCTGCTACATGCCGATCTTTCTTAGTTTTCTCCTAGTGAATTGT |
| | | | | AATTTTTAATATATAATTTGTTATTCCACTATGTCTGTTCCTGATTTAC |
| | | | | TTTCGCTGATCACATCGTTCGTGTTTCTTCTATTGTAGCTGATGTGGT |
| | | | | TGGTGGACATCTTTCGTGCCAAAATTGTGGACATATCGGAAGAGTA |
| | | | | TCTTTCAATAGAGGTAATCACAGGATATTTCATAAAATCAATGTTCA |
| | | | | TTTTGGGAATATTGGGAATATTGTGACAAGTATCAATGTTTTATGTA |
| | | | | CAGGTTAAGTGTTTTATTTGCATTTTCCACAAGCTACTTCATCCTCTT |
| | | | | CTCCGAAGATGTGTAAAATTGTAATATACATATTCACACCAAATTTA |
| | | | | AAATTTTGGAAGAGCGACATACAAATGATTTATGACCTAATTTTGCT |
| | | | | GTATATATACATGCTTATATTTTCTTCAAACCGCTTTAGAAAATTTAT |
| | | | | TATATTTTTCCTCCTTCAAAGAAAACAAGTTTTGTGAGATAAAAGCT |
| | | | | ACATGTTTAGTAAACAAATTTAGTGAAGACTGATGAAAACAAAATT |
| | | | | TGTACTTTTGAATGGTTTTATATATTTTATGATCTTTGTAAGCTAATA |
| | | | | TGGAAACTATAAGATTGTAGTTGGTTTTCTTTAGCTCTTTTCGCTTTT |
| | | | | ACCTTTCATTTTTTTTACATGCTTAAAAAAGCGACTCACCTAGTACTT |
| | | | | TGATATCCAGGTCACTGGAGATCCAGGAAAGATGGTTGCTGTCCTT |
| | | | | AGAAACCTAAGCAAGTTTGGCATCAAAGAAATTGCTCGTACCGGAA |
| | | | | AGGTTTACTATTTTTTTTTGTAAATTTTTCATCGTTGTTAAAGAGATC |
| | | | | TTTTAGTTTCCACATTGCATAATAAATGTGAATGCCTTTTGTTTATTG |
| | | | | CTTGTCCAGGTTTTTTATATTTTTTTAAGATGTAAAGTTTAATATCTA |
| | | | | ACAGATTGCTCTAAGAAGGGAAAAATTGGGCGAGTCTGCTCCTTTC |
| | | | | TGGCGTTTTTCTGCTGCTTCTTATCCTGATCTTGAAGAAGCTATCCCT |
| | | | | ATGGATTCTCTTTCTGGAGTTGCAAAAGCAGCAACTGCTGCTGGATC |
| | | | | ATCGGATTCGTCTGTGGAGGTGTGTTTCTTATTTGTCCAAAATTAAG |
| | | | | TGTCCAAGTGTCGTGCCCAAGTCCGAATATCGAGGATCAGACATGG |
| | | | | GTACTTGAGTTAAAATGAAGAGTCCGGGTAACAAATGTAATTGATT |
| | | | | AAAAAACATATAGTTGCCGGATTTTAAATTTTTTTTTAAGGTAGTTG |
| | | | | GAAAAATTTCCTTTTATTAGAAAAACTTTGGGAGGACAATATTTTTG |
| | | | | TGAATGTGTCCGAGTGCCTCTTATGTTAAAGGGCCTCAAGACGAAT |
| | | | | AGGAAATGGCAGGCGGGTCGAATGCGGGTGTGCAGGATGGTTGCT |
| | | | | GGCAATGTTTCCGCAGGCTGTTGTTGGTGTGCTTTTCACTTCAGTAG |
| | | | | CAATTTACAGCAAATTAGTAGCAAATTTACATCAAATTTACAGCAGA |
| | | | | TTGTTAGCTCAATAACCCATCATGACATAAATATTAATGTATAACAT |
| | | | | AACTTTAACTACAAAATACAATTTAGAATCATCAAATCACAATTTAG |
| | | | | TTGTTTAATATACATTAAAATATTTCTTTGTAGGAACTTCAATTCTAT |
| | | | | CTCTTAGTAGCATAAATTTTACAATCAAACGCAACAAAAGTATTAAG |
| | | | | TTAGAAAGGGAGTACTGCATGTGGATTGTCTCAGCAAGTGCTGCAG |
| | | | | CAGCTTTGCACTATCTAGTGAGACATTGGAAATGATTAGGAGCTTT |
| | | | | GGGATAAATATGCGCAGAAACACTTCTCTTCTTGCCATCCCAAGGAT |
| | | | | ACTGCAGCTTCAGGAAAATGAAGGTCCTGTATGATTTTTAGAAACTC |
| | | | | CACCAAATTCTGAAGATTAAACTCACCTCCGTACCTAACATTGAGAT |
| | | | | ATCTTCAGTATATTCTCACCTACCTGATACCTTGCTATGAGATCTCTG |
| | | | | TAAATCAGCTTCACTAAGACAAGCCAAAATTTCTAACTAGAAGTCAT |
| | | | | TTTCTGCAAATTTTAATGCAGATTCCAGAACTACCCATTATCATTAAA |
| | | | | CTTGAATATTTCCCCTTTTTATAAC |
| 1708 | Amaranthus | gDNA | 4516 | AAGAAAAGTGTAGACACAATTAAAAAGTTCACTTTGAATAGGAACC |
| | palmeri | Contig | | AAGGAAATCTAGGTAAATTATAATTATTTAGGAGAATGGTAAATCA |
| | | | | TCACAATGTGGCCTAATTTTATAAAAGGATATTTGGTATTTTTTTTAA |
| | | | | TATTTTTCTTGTATTAATTATTTTATTAAAATGACTGATGATTTCTAAA |
| | | | | TCACCATTATTGATTAAGATTTATATAAATCACATTGTAAAAGCAAA |
| | | | | ACAAAAAGCACAAATTCAATAATATACTCTTTAAGTTTGTTTATCTTC |
| | | | | TAATTAGTTCGGTTAAAACGGTTCCCCACTTTCTTCTCCGACTCTCAC |
| | | | | AATTATCTTCCCCTATTCATTTTTCTTCCACCCTCTCTAATGGCGGCTG |
| | | | | TTTCCTTCAATATCAATGGTGGAAAGATTGGAACTTTATGTTCAAGA |
| | | | | CACGAATTCGTTTGTGGGTTTGTAAGAAAATTTCATTTTAGAACTCA |
| | | | | TACTTCTATATTTGAAAAACATATGCCAAAAACTTCAAGGTTTAAAG |
| | | | | CAATGGAAGTTTCTGCAAATGCAACAGTAAATATAGTTCCTGTTTCA |
| | | | | GCTCATTCTAGGTAATTTTATTTCTCGAAAATTTCCGATTTACAATTA |
| | | | | AATTAATCTTGTTTTGTAGGTAATGAATTGCAGAAGAAATAGATGG |
| | | | | ATTCTTATTTGTTTATTGGTATTTGTTTATAAATTTTTGTTTATATTAG |
| | | | | TTTCTGAATTGTGATTATTCTGATTGTATGTCAAGGTTTAGGTTGTTA |
| | | | | CTAAATGTAAATTGGATTGATTGAAGTTGCAATAAGGTGATGGCGT |
| | | | | GATGCTGATTGTTGTAAATTTTTGTTTATATTAGTATAGTACTACTTA |
| | | | | TTGACCTAAGTTCTTTTACATAGTAGGAGATATTATAACTTTTGCTTG |
| | | | | TGGACTCAGGGCCGGCCCTAAGGGTGGGCAAGAGGGGCCGTCTCC |
| | | | | CAATGCCCATGTCAAAAAAGCAAAATTAATGGTTATATAAGGTTTAT |
| | | | | ATAGGTTATAAATTGTAAAGGAAAAGGACGTCAATGTGTCATTTCT |
| | | | | CCCAGGGCCCCAAAATATCTAGGACCGGTCCTGAAAGAAACTACTC |
| | | | | AAAATTTGTAGGATTTTCTATTGAGACATTTTTTTTTTTGTGGATTAA |
| | | | | TGATTGAACTAAGATCAAATAGATACTATTTCTGTTTGATTGAAACT |
| | | | | TTTGGGTAGCCGGATGTGTACCGGCGTGTGACTTCTTTTTGAGTTG |
| | | | | GTTGATTCAATTGTATTGTTAGAAATGATCTTTTCTAGGGTCTTTGG |
| | | | | AGACATTGGTTCTTTACTTTTAGTCTATGAATTATGATAGTGTTGGTT |
| | | | | TAAACACCATTTTGTTACCCTATTGTGATTAAGTGACTCCCAGCTCG |
| | | | | ATTAAATTTAGGAGTTATATTAATGTTGTCTTTGTTAGTTATATTGAT |
| | | | | GGTCGGTAGTTCGGTTAAGTTTGTCTATGAATACATTGATTTTATCC |
| | | | | TTATTTGGCCAACAAATGTATGTAATTTAGGAAAGTTATATGATTTA |
| | | | | CCACTTTGTATTTGTATCTCTGTTATTTTCTTTCTTCTTTTCTTGATGTG |
| | | | | TTGGTTGGATAAAAGGAATTTGGAGGGAAAGAAAGGAAAGAGAA |
| | | | | ATAGAGGGATGAGACTTTGTCATATGTATATCAAATAGAGATGGGA |
| | | | | AATGGAGGGGAGGGAATAATAACAATGAATTCCTTCTGTTGTTTTT |
| | | | | GAAACCAATTTCTCCATTATTAGAAGGAACTCATATCCTTGTGTATG |
| | | | | ATCGGAGAAAGTAATGACTGAATCATTCATGAAGCTCATCCTTGGT |
| | | | | GAATTTATCTCTTGCTAATTTCTCCCATTTTAGAATGACCCCGGTCAC |
| | | | | TACATTTATGCTAAGTCTGATTGCTCGGGCCGTTGCCTACTAATCAT |
| | | | | TCCAAATCTCTTGAATAAAAGTCATTTTAAATTATCAAATGTATCTCA |
| | | | | TTCTATGCTGTTTTTTTTCTTGTATGTTGTTATCATTAGCATGGTAAC |
| | | | | AAAATGTATTTTCATTCTTTTCCAGGGTGATGCGCCACACAATTTCA |
| | | | | GTCTTTGTTGGGGATGAAAGTGGTATAATCAATAGGATTGCCGGCG |
| | | | | TTATTTCTAGGAGAGGATACAACATAGAGTCTCTAGCTGTTGGTTTA |
| | | | | AATAAGGATAAGGCTCTTTTTACTATAGTAGTGTGTGGGACTGACA |
| | | | | AGGTGTTGCGCCAAGTAATGGAGCAGCTCAGCAAGCTTGTTAATGT |
| | | | | CTTAAAGGTAGTCATCTCCTTTTTTCTTACTTTAAGGTAACTAGTGAT |
| | | | | ACTTTCATTTAGTAGTGGCGGTCAATGGTGATGCTAACCATGCATGT |
| | | | | TCATATTGTTTGTGCAATATTGCTCATTTTGGCTTTATCATTGTTGTT |
| | | | | AAACTTTGAGAGAATGTAATCTCAAGAGCATCTATCCTCACTTCAAG |
| | | | | ATAGATAGAGCAGGGAAACACTCATGATTCTTTTAATAAACTTTCAA |
| | | | | AATATTAAAATTCCCTTGCCCTAATGCAATTTTATAGCTCCCAGCTAA |
| | | | | TCCGAGTTCAAGGGGTTCGAGTGTATGCAACCATACTCATATAACA |
| | | | | ACAAAATGGTTGATTCAGATTGACCCTTAAAATCAAATACCATCTTG |
| | | | | CAAATTCACTTAAACACATACTTTAACGACCCGTTTTATAGTAGTCCA |
| | | | | TTAAAATTCGAAAGCAAAGAATTATAAAATCTTTTATTCCATCGAGA |
| | | | | ATAGACACAATAATCTTCAAAGCAATGTCTATAAAAAGTTTGTCGTG |
| | | | | TCACCGTATCGTAACTTAATTGTAAGGGCCTTCAATTTTACTGCCATC |
| | | | | AAAATATAGGTTGAGACCCACAAAACCATTTACATAGACAAGAGCC |
| | | | | ATTTCACTACCATTATCATGACCATGATTGAGACCGCATTTGTGCAC |
| | | | | AATGGTCAAGTGACATTGTTCAGCACTACTCAGAACTTGAATTGGA |
| | | | | GGAATTTGTCTAATGATCTACTTATGTAACAAATTCTTGATAATGTT |
| | | | | GTCACATATAGTGGGGAGAAGTTTGAAGGTCACATAGCTACCACAT |
| | | | | GTAACTGAATGCATCTATTGCAGTTAAACCTAATGGCAGGGAGCTG |
| | | | | TCAATGCAGTCTTTTGAAAGGAACTTGTAGAAGGTATCTTTTAAAGA |
| | | | | AAAAAAATTTGTCGTTAGTTTTTAAAAAAAGTTAAAAATTATTTTCTT |
| | | | | GAAAATTTTCTTTTTAAGTAAAATAAGTGTGGTAAAATGGACATTAT |
| | | | | GTTGGAACTTAGAATTTTTAGTAATAGCTTGTTAATTAGCATGGGAA |
| | | | | GTGATTAATGAAGTTTTATGTCCATTAGGTAATAATGTGTGAAGTAT |
| | | | | TCTTATATGTATTATTGCTTGCACTTTCCATTTGATGGTGCGCATCAG |
| | | | | GGTGTGCAATAGTACTTTATCTGATCTGACATATATGTTCTTACTTAT |
| | | | | GCTATATGCTCGCATTTAAGCATTCCTTACTAAAGGTGTTTAACGGG |
| | | | | ACGATTCGACTAATGGATCAACGGTCGGGTCGGGTCATATCTTTAA |
| | | | | CGGGTCATCAGCGGGTCAGGTTAATAAAGGGTCGAACCATTAACG |
| | | | | GGCCTTGATGTAAAGGGTCGGGCAGAGCCGGGTCAAATAATTATA |
| | | | | GGAATTGGTTGCAAAAAATTTTTTTTCCACTGTTTGTTTTTATATTTT |
| | | | | AGATGATATTATATACATAGTTAGGTCGATCCAACGGGCCATAAAG |
| | | | | TAGAATAAAAAAAACTATTACATGAACCTTTAAAAACGGGTCGAAG |
| | | | | TGGGCCTGGTTTAAACGGGCTTTGACCCGTCCTGCCCCGTTGAAATT |
| | | | | TGACATGACCCTCCCCGCCTCGCCCTATTTAAATTTAGCTCCATCCCG |
| | | | | GCCCATTGAACACATCTATTCCTAAAATAACTAACATTATGATGGCT |
| | | | | CATCCTATTCGCTACCATGCATGTAAATGTACACAGTGTCTATCCGTT |
| | | | | TATCCTTTTTTATATCCCTGTATACCATTTGTGACTAACGGTATTAAT |
| | | | | TTTTTATCTTCCTTGTAGGTTGAAGATCTATCTAGAGAGCCTCAAGT |
| | | | | GGAACGTGAACTTATGCTATTAAAGCTTCATTCTAATGCGGATACCC |
| | | | | ATGCTGAGGTAGTTCCACAAGTTCAAATAATTATATCCTGAATGCTA |
| | | | | TATAATTCATGAATCTTTTGTTTTCCTCTTAGAATCATCAGCTACAAG |
| | | | | AAGGTAGATAAAGATAAGCAAATATTCAAAAATATGCTATAACATC |
| | | | | ATGTGCTTTGTAAGCTTTAAACTTAGAGATAACAAAAATACTTAAGG |
| | | | | ATACATCATGGGTATGTAGCTGAAGATCATATGAGAAGTTACATTG |
| | | | | CTTCTTTGTGTGTATCGTAAACAATCTGGTATTGTATAAGTCCAAATT |
| | | | | CCATATTTTGATTTTCAGATAATGTGGTTAGTGGACATCTTCAGGGC |
| | | | | AAAAATTGTCGATATGTCGGAAAGCTTCGTCACTGTAGAGGTTAGT |
| | | | | ATGTTTGTGATTGACATCTTGTGTACCTATCTTCTGTTCAATGTAGAT |
| | | | | TCTTTTTCGTAGTGGTATTTTTTTTTCTGTCGTGCAGGTGACTGGCG |
| 1709 | Amaranthus | cDNA | 1767 | GTGTCTTTCTTTGCACCCTGGTTGTCCTCGTCAGGCCGAGTTAGCTG |
| | rudis | Contig | | TGTTTCAAATTTCCATCCATGGAGGCTGTGTCGACTCACCTTTCAAC |
| | | | | GAGTTTTAACTCCATTCCGAAAAGTAATAGATTGAACCATCAAACTG |
| | | | | CAAAACGATTAGGGTTCTCCTTGAAACCCCATTCGCTGGGTTTTAAG |
| | | | | TTAACTCCAATAGAGGTTGGAATTTGGAGTTTGATAAACTGGTTGTA |
| | | | | TCTGCAAGCAATGTTGATCAACTGGGAAATCAAAGTAACTTATCCTT |
| | | | | TAATCCCCTTTCTCCCTCTCGATCAAAGGAGAGACGACATACAATAT |
| | | | | CAGTATTTGTGGGGGATGAAAGTGGAATGATTAATCGAATAGCAG |
| | | | | GGGTCTTTGCCAGAAGAGGTTATAATATCGAATCACTCGCTGTTGG |
| | | | | ATTGAACAAGGACAAAGCTCTCTTCACTATTGTAGTTTCTGGAACGG |
| | | | | ATAATGTGTTGCAGCAAGTGATGGAACAGCTTCAAAAGCTTGTCAA |
| | | | | TGTTTTGAAGGTTGAAGATATATCCAAGGAGCCTCAAGTAGAACGT |
| | | | | GAATTGATGCTTGTAAAAGTTGGAGCTGATCGGAATAACCGTGCTG |
| | | | | AGCTGATGTGGTTGGTGGACATCTTTCGTGCCAAAATTGTGGACAT |
| | | | | ATCGGAAGAGTATCTTTCAATAGAGGTCACTGGAGATCCAGGAAAG |
| | | | | ATGGTTGCTGTCCTTAGAAACCTAAGCAAGTTTGGCATTAAAGAAA |
| | | | | TTGCTCGTACCGGAAAGATTGCTCTAAGAAGGGAAAAATTGGGTGA |
| | | | | GTCTGCTCCTTTCTGGCGTTTTTCTGCTGCTTCTTATCCTGATCTTGA |
| | | | | AGAAGCTATCCCTATGGATGCTCTTTCTGGAGTTTCCAAAAGAGCA |
| | | | | GCTGCTGCTGGATCATCGGATTCGTCTGTGGAGGGTGATGTTTATC |
| | | | | CCGTGGAGCCGTTTGATGGTTTCTCCCCTCCAATCTTAGATGCTCAT |
| | | | | TGGGGTATTTTGAATGAAGAAGATACTAGTGGGATGCGGTCACACA |
| | | | | CTCTATCTATTCTTGTCAATGACAAACCCGGGGTCCTTAATGTTGTTA |
| | | | | CGGGGGTTTTTGCTCGAAGGGGTTATAACATTCAGAGTTTAGCTGT |
| | | | | GGGTCATGCGGAAGGTGAGGGTCTATCTCGTATCACTACTGTTGTA |
| | | | | CCCGGTACAGATGAATCAATTAGCAAATTGGTTCAACAAATCTACA |
| | | | | AGCTGGTTGATATTCATGAGGTTAGAGATCTTACCCATTATCCATTT |
| | | | | GCTGAGCGAGAGTTGATGTTGATAAAAGTAGCTGTGAATACTGCTG |
| | | | | CACGTCGTGAGGTCCTAGACGTTGCCAGCATTTTTAGAGCAAAAGC |
| | | | | TGTTAGATGTATCTGATCACACCATAACACTTGAGCTCACTGGAGAT |
| | | | | TTGAACAAGATGGTTGCTCTACAGAGATTGCTCGAACCGTATGGAA |
| | | | | TCTGTGAGGTTGCACGAACAGGACGTGTGGCCTTGAGTCGAGAGTC |
| | | | | TGGTGTAGACTCGAGATATCTCCGTGGATACTCTTTCCCTGTGTAAC |
| | | | | AGTTCTGTCAAAACGTATGATGTAAAAAGCCTTGGTTATTTTTTTTGT |
| | | | | TTTACGAAATTGTCTCCAGATTTTGATCCTTTGAATTTGTGTTCTTGA |
| | | | | AGAATATTTGCAAGTTTGGACCACATCTGAAACTTTGTTTTAGTGAA |
| | | | | ATGAGAGACCATCTTCTACCTGGTGAAAAGCAGTTATACAAAAAAA |
| | | | | AAAGAAAAAAAAACAAAACATGTCGGCCGCCTCGGTCTCTACTGA |
| 1710 | Amaranthus | cDNA | 1560 | GGTATAATCAATAGGATTGCCGGCGTTATTTCTAGGAGAGGATACA |
| | rudis | Contig | | ACATAGAGTCTCTGGCTGTTGGTTTAAACAAGGATAAGGCTCTTTTT |
| | | | | ACTATAGTAGTGTGTGGAACTGACAAGGTGTTGCGCCAAGTCGTGG |
| | | | | AACAGCTTAACAAGCTTGTTAGTGTCTTGAAGGTTGAAGATCTATCA |
| | | | | GAGAGCCACAAGTGGAACGTGAACTGATGCTTTTAAAGCTTAATTC |
| | | | | TGATGCAAATTCCCACGCAGAGCTAATGTGGCTAGTGGACATCTTC |
| | | | | AGGGCAAAAATTGTGGATATCTCAGAAAGCTTGGTCACTGTTGAGG |
| | | | | TGACCGGAGATCCTGGAAAGTTGGCTGCTGTCCTAAGAAATTTTAG |
| | | | | AAAGTTTGGAATCAAAGAAATTGCAAGGACCGGTAAGATTGCTCTA |
| | | | | AGGCGAGAAAAGATGGGTCAGACAGCTCCTTTTTGGAGATTCTCTG |
| | | | | CTGCTTCTTATCCAGATCTACAAGAAAAGGCTGTTGATGCTCTTGCC |
| | | | | AGGCCTATGAAACGGAGCGTCAATGGTGATTCTGGCTCATCTTCAA |
| | | | | GTGGTGATGTTTATCCGGTGGAACCTTATGATGTTTCGATGGTAAAT |
| | | | | CAAGTACTTGATGCTCACTGGGGCGTACTTTATGACGGTGATTCAA |
| | | | | GTGGTCTCCGGTCACATACGCTGTCCATGCTGGTAAATAATGCTCCC |
| | | | | GGAGTTCTTAACACTGTTACAGGAGTAATTTCTCGTAGGGGTTATAA |
| | | | | CATTCAGAGTCTTGCTGTAGGCCCTGCTGAAAAGGAGGGTCTTTCTC |
| | | | | GTATCACAACTGTCATTCCTGGAAACGATGAGTCGATTGGAAAATT |
| | | | | GGTTCAGCAATTTAACAAGTTAGTAGACCTTCATGAGATTCAGGAC |
| | | | | CTTACGCACCAGCCATTTTCAGAGCGAGAGCTTATGTTGATCAAAAT |
| | | | | AGCTGCAAATACTACAGCCAGGAGAGATGTTCTTGATATTGCTAAT |
| | | | | ATTTTCCGTGCAAAAGCTGTGGATGTTTCGGATCACACGATAACATT |
| | | | | ACAGCTTACGGGTGATTTACACAAAATGGTTGCGCTACAAAGATTA |
| | | | | TTGGAGCCTTATGGCATTTGTGAGGTGGCACGGACAGGAAGAGTA |
| | | | | GCACTGAGTAGAGAGTCTGGGGTCGATTCTACATCTTTACGTGGAT |
| | | | | ACGCTCTTCCATTGTACGAATGAAAAATCTCCGGCTATGTCTTTCCG |
| | | | | TCAATATCATGTCCATTTCCTTCAGCACGTCTGCACTACATTTACATA |
| | | | | GCGATCAATCGCCCGTCAACCAGAGTGAAGGAAAGCAGTATTTCTG |
| | | | | ACCTTTGCTTTGAACATGCTATTTTGTATAATAGGAATAATTTTGTAT |
| | | | | TGGTTATATTTGCTAATACCCAGTAGTAATATTGTTACCTCGAGTTC |
| | | | | GAATATCTGAAACATACTTTTTCTCTTGAATGCGTAGAGAACATTAG |
| | | | | CACGGTCTCTTGGCCGGATAACACGCATTATTCAAGAAGGAATTAG |
| | | | | GATTGTAAACAGTTCCAAACACTAGTGTTTTCGCGTTGGGAAAACA |
| | | | | AAAGAAAAAAAACAAAACATGTCGGCC |
| 1711 | Amaranthus | cDNA | 341 | GGTATAATCAATAGGATTGCCGGCGTTATTTCTAGGAGAGGATACA |
| | rudis | Contig | | ACATAGAGTCTCTGGCTGTTGGTTTAAACAAGGATAAGGCTCTTTTT |
| | | | | ACTATAGTAGTGTGTGGAACTGACAAGGTGTTGCGCCAAGTCGTGG |
| | | | | AACAGCTTAACAAGCTTGTTAGTGTCTTGAAGGTTGAAGATCTATCA |
| | | | | GAGAGCCACAAGTGGAACGTGAACTGATGCTTTTAAAGCTTAATTC |
| | | | | TGATGCAGATACCCATGCTGAGATAATGTGGTTAGTGGACATCTTC |
| | | | | AGGGCAAAAATTGTCGATATGTCGGAAAGCTTCGTTACTAGAGGTG |
| | | | | ACTGGCGATCCTGGAAA |
| 1712 | Amaranthus | gDNA | 7847 | GTATCTGCAAGCAATGTTGATCAACTGGGAAATCAAAGTAACTTAT |
| | rudis | Contig | | CCTTTAATCCCCTTTCTCCCTCTCGATCAAAGTATGTTATTTTTCCCTT |
| | | | | TTTTGATTAATTTCACAATGTTAGAATCTGGGTTTGGCTTGTTGTTTT |
| | | | | AATTTCTACTAGTTAATTTTGTTTTTCATTATGTTATAAAATTTTTAAA |
| | | | | TGAAAATGAGTAATCATTTAATTTATGAACAATTGTTGTCACTCGTG |
| | | | | CTAGCCTGAAGGATTGGGAAAATTAGTCAATTATGAATCTGGTATA |
| | | | | TTTTTCATTGATTTGTGTGTTTTGTTGCATCTTGCATGTAGTTTGTTCA |
| | | | | TCCATTTGATTTTTATACAAGAGAGCGTGCAAAATTTAGCATGTTTT |
| | | | | CAGTTCTTAAATTGATCACTGCCTGTATTTTATTAGCGTACCCCATTA |
| | | | | AAACGACTCATTAAATCACTTTTTTTATTTGGGTGAAGATGTTTAAG |
| | | | | ATCTTTGCTACAAAGATTTGATCGGAAACAGTCTCTTTGTTATTATCA |
| | | | | CTAATAAGGGTAAGATTGTATACATTAGACTCCAAACCGCACCTAG |
| | | | | GTCTGGGGGCACTAAATAGCATTGGGGTAATGAAATGTTGTTGGTG |
| | | | | TATTTTTGTAATGAATACCAAACAAGCTGTTAAGTATAGATACTTTC |
| | | | | CCTGCAATAATACAAGAGTTTATGAGGCTCTTGTTCAAACCAGCACA |
| | | | | AGTTTATTGAAAGATTTATCAGTAGTTTTTTTGTGATTCATAATCTTT |
| | | | | TAAGTACCAAGCAAAGCTCTTTATGACATGGAAACCCGCTGCAATCT |
| | | | | AATTGACCTAGTGCTAAAAGCTGCTTGATTTGCTTATTGCCTTAAGT |
| | | | | GAAAATTTATAGCAAGCGTTTTGGAGGAAGAAAAGCAAAAGATCG |
| | | | | ATATGCTTTCATGTCTTCAGAAAATTAGTTCCGAAGTTGTGGAGTAT |
| | | | | AATAGAGTTCAATTATTGTTTGCATTTAGATCTACCTTGATTTGATAT |
| | | | | ACAACAACAATGTCATATCCTTATTCCTAAAAGATTAAATTCTACTAC |
| | | | | ATGAACCAATATGCTATCATTTTGAGTAATTGTCCTTCTCTATTCATT |
| | | | | TCGATTTTCTACCACTCAACTTATAAGTCTAAATCTTTCATATGCTTTT |
| | | | | CCACTCCTGGTCATCTTTGCTCTTCCTCACCTTCTTTTTAAGTCTTCCG |
| | | | | ATTTGCTACTTTCTATCCTTCTTATCGGTTCACTTATACCACATCTTTG |
| | | | | CATATGCCTAAACAATCTTTAACAATTCTCTAGCGTTGTTTTCTCAAA |
| | | | | ATTTGCGACTTTCAAGCCTTTTTTTACATATTTTTTTTCGAATTCTATC |
| | | | | ACTCAAGGTATACCGCTCATCCACCAAAGCATTCGCCTTTCTGGTAC |
| | | | | TTCTATCTTTCTACTATGATCCTTTCTAAAAGCCCGCGTTCAGATGTA |
| | | | | TATAGTAGCGTTGATCTAATGGTGTAAAACTTACCTTTTAGCTTTAG |
| | | | | GGTCACACCTCGATCACCTAATGTCCTAGTCGTTCTTCTTTTGCCACC |
| | | | | GACACTTTATTTATTGTGTCACATCTTGTTGGATATTACCATAACTCT |
| | | | | AGAATCATACCCAAGGTATTTGAAACATCTACTTGGAGTAATTTCTT |
| | | | | TCTCTTCCAACATTATAATGTCGATTTATGTGATTTGTGCTAAAGTGA |
| | | | | CACTTCATGTACCCTATCTTCCATCGACTTAATTTAAACTATCGTGAC |
| | | | | TTCAACTCTTGTCTTCATCGTTATAACTTAGCTTCTACCCATTCCGCG |
| | | | | ACTTCATTTTCATCTACCTTGATATACCTTGATATAGCTTATCATTAAT |
| | | | | ATTTTTCTCATAATAATTCAAAAGTTTGCATCTATCTAATTTTAGTGC |
| | | | | TACAAGTGTTGCTTGTAGTAATTCTGTCTTGTTAGATTGTGGTGTGT |
| | | | | GAACTATGTATTTTTCGTGAATTGTTGCATTTCTTGAATGACTTGTCT |
| | | | | CTAGAATGATGCTCCTTAAACTTACTTCTTTTGTTTCATGTATGTTTA |
| | | | | AGGGAGAGACGACATACAATATCAGTATTTGTGGGGGATGAAAGT |
| | | | | GGAATGATTAATCGAATAGCAGGGGTCTTTGCCAGAAGAGGTTATA |
| | | | | ACATCGAATCACTCGCTGTTGGATTGAACAAGGACAAAGCTCTCTTC |
| | | | | ACTATTGTAGTTTCTGGAACGGATAATGTGTTGCAGCAAGTGATGG |
| | | | | AACAGCTTCAAAAGCTTGTCAATGTTTTGAAGGTTATTTTCCTTTAG |
| | | | | AATAATTGCCGTGGCATCCATGTCTTCAATTGTTTAGAATTCTCATCA |
| | | | | CTAAGTACAACTTATACATATAGGTTGAAGATATATCCAAGGAGCCT |
| | | | | CAAGTAGAACGTGAATTGATGCTTGTAAAAGTTGGAGCTGATCGGA |
| | | | | ATAACCGTGCTGAGGTACCTAATAATATATGTCACAGTACCCAACTG |
| | | | | TACAAAATAGTTGATCTTATTAGATACAGACAGATATATTAGCTAGA |
| | | | | TCTCTATGTTTTTTGTGGTGACTATTGACTTTCTGCTACATGCTGATC |
| | | | | TTTCTTAGTTTTCTCCTAGTAAATTGTAATTTTTAATATATAATTTGTT |
| | | | | ATTCCACTATGTCTGTTCCTGATTTACTTTCACTGATCATATTGTTCGT |
| | | | | GTTTCTTCTATTGTAGCTGATGTGGTTGGTGGACATCTTTCGTGCCA |
| | | | | AAATTGTGGACATATCGGAAGATTATCTTTCAATAGAGGTAATCAC |
| | | | | AGGATATTTCATAAAATCAATGTTGGTTTTGGGAATATTGTGACTGC |
| | | | | AGTTTTACTTAGACAAGTATCAATGTTTCAGGATGTTCTACAGGTTA |
| | | | | AGTGTTTAATGTACATTTTCAACAAGCTACTTGATGCACTTCTCTGAT |
| | | | | GACGTGTAAAATTGTAATGTACATATTGCACACTGAATTGAAAATTT |
| | | | | TGGAAGAAGGGACATTAAAATGATTTATGACCTAATATTGCTGTAT |
| | | | | ATATACATGCTTATATTTCTTCAAGCAGCTTAAGAAAATTTATTATAT |
| | | | | TGTTTTTCCTCCTTTAGTGAAAAAAGTTCAGTGAGATAAAAGTTTCA |
| | | | | TCTTTCATGTTTAGTAAACAAGTTTAGTGAAGACTGAAGAGAACAA |
| | | | | AATTTGTACTTTTTAATGGTTTTATATTTTTTGTTAATTTTGTAAGCTA |
| | | | | ATATGGAAACTATAAGACTGTAGTTGTTTTTGTTTAGCTCTTTTCACT |
| | | | | TTTACGTTTGAGTTGTTTTTCAATGATCACATGCTTAATAAAGTAATT |
| | | | | TACCTAGCACCTTGAATATCCAGGTCACTGGAGATCCAGGAAAGAT |
| | | | | GGTTGCTGTCCTTAGAAACCTAAGCAAGTTTGGCATTAAAGAAATT |
| | | | | GCTCGTACCGGAAAGGTTTATTTTTATTTTTTTTGAGAATTTTTTTTA |
| | | | | TCTTTGTTTAAGAGCTCTTTAAGTTTCCATATTACATAATAAATCTGA |
| | | | | ATGTCTTAGATATAAAACTTTTGTTTATTGTTTGTCCAGGGTTTATAT |
| | | | | ATTTTTAATATGAAGTAAAGTTTAATTTCTAACAGATTGCTCTAAGA |
| | | | | AGGGAAAAATTGGGTGAGTCTGCTCCTTTCTGGCGTTTTTCTGCTGC |
| | | | | TTCTTATCCTGATCTTGAAGAAGCTATCCCTATGGATGCTCTTTCTGG |
| | | | | AGTTTCCAAAAGAGCAGCTGCTGCTGGATCATCAGATTCGTCTGTG |
| | | | | GAGGTGTGTTTCTTATTTGTTCATTGCTCACATTCCTAATATTTTGAG |
| | | | | AGTTGAATCCACACATTTCTTTCTTGTTGTACAATTCTTTTTGTTTCTT |
| | | | | GTTTCCATCTGCCCATTTACATGATTGCATCTTTAATTATATTATTATT |
| | | | | GCTTTCACTAAATGGAACACCTTGATGTTGTGCTTTCCAAAACGAAC |
| | | | | GTTTTTATTCAAATCCATTCCTCAATACCAGTAATAAACCGGATTAAA |
| | | | | AGTGAACCACTATTATGGGAGGTTTTAGGGTATATTATGGATTCCG |
| | | | | ATTAAAACATAGTTAAATCCTAAAAGGTAGTAATTGGCTATGTTATC |
| | | | | TGGACTCGGGAACTCATGTGATATGTGTCGGAGTGTCTGATTCGGC |
| | | | | TAAGTTAAGGAAAAATACAATAACTTTTGTCCAAAATAAAGTGTCCA |
| | | | | AGTGTCGTGCCCAAGTCCGAATGTTGAGGATCCGACATGGGTACTT |
| | | | | GAGTTAAAAATTAAAATGAAGAGTTCGGGTAACAAATGTAATTGAT |
| | | | | TAAAAAACCTATAGTTGCCAAATTTTTAAACTTTTTAAGGTAGTTGG |
| | | | | GAAAATTTCCTTTTATTGAAAAAACTTTGGGAGGACAATATTTTGTG |
| | | | | AATGTGTCTCGAGACATGGCTCTTATGTTAAAGGGCTTCAAGACTA |
| | | | | ATAGGTAATGGTGGATATGTCGAATGCGGGTGTGCAGGCAGGTTG |
| | | | | CAGGTCAAATATTTCCACAGCCTGTCGTTGGTGTGCTTTTCACTTCTA |
| | | | | GCAATTTACAGCAAATTAGTAGCAAATTTACAACAAATTTACAGCTG |
| | | | | ATTGTAAGCTCAATAACCCATCATGACATAAATACTAAAATATAACA |
| | | | | TATGTTTAACTACAAAATACAATTCAGAATCATCAAATTACAATTTA |
| | | | | GCTGTTTCATATACATACACTAAATATTTCTGTGACCCAACTTCAATT |
| | | | | CAATCTCTTAGTATCATAAATTTAACAATCAAACACAACAAAAGTAT |
| | | | | AAAGTTAGAATGGGAGTACTGCATGTGGATTGTCTCTCAGCTAAAG |
| | | | | TCCTGCAGCAGCTTTGCACATTATCTGGTGAGACATTGGATATGAGT |
| | | | | GAGAGCTTTGGGATAAATATGCACAGAAATACTTCTCTTCTTGCCAT |
| | | | | GCCCAGGCTCATACTGCAGCTTCAGGAAAATGAAGGTCCTGTATGA |
| | | | | TTTTTAGAAACTCCACCAAATTCTGAAGATTAAACTCACCTCGCTAA |
| | | | | GCAACATTGAGATATCTTCAGTATATTCGCACCTATCTGATACCTCA |
| | | | | CTATGAGATCTCTATAAATTCAGCTTCACTAAGACAAGCCCAAAATG |
| | | | | AAATTTCTAACTAATAGCCATTTTCAGCAACTTTTTATGCAGATTCCA |
| | | | | AACTATCCATTATCCATAAACTTAAATATATCCCATTTTTATGACATT |
| | | | | TTTGATGCCTCTTGTATGCTAATTTTTTCTAACTATATACACTACCAC |
| | | | | AAAACACAAATCTATGCTTACTCTAAATAAGTTTAATATGAACTACA |
| | | | | TTTTATTTTTAGTGACTACGGCTTAGAAGTATATCTTAGAACAACTTA |
| | | | | CATTGTAGATTAATATGACATAATCTTTTATCAATCTATACCTCCTCA |
| | | | | GGACAAGTAAGATAAAAGAACTCTAATGTACAAAATTTAATTGATT |
| | | | | TTTTTGAAAAAAGATACAAGAAAATGGAAGAGGCAACACAGGCAA |
| | | | | AGAAGGCTTCTCGATCCTCTGAGCACTCTAAACCTTGCAATACTACA |
| | | | | GTGAACTATAAAAAAAGACAAGAAAGTTCAGAAATCATCAAAATAT |
| | | | | TTTCCCCTAAAAAGGTTCAACATCATCTTCTTCCTCTTCTTCCACAATC |
| | | | | TCGAATGTCATGTTCATCTAAAAGCCTATCCCTGTCCTCAATGGTGTT |
| | | | | GTACATAGCAGGCCTTGGAGGCACACTAATGCTTGCACTGCTTTTG |
| | | | | ACTTCAAGGAACTTTGAGTAGCAAAAATTGTCTTTTCACCTTGGCCA |
| | | | | AAGGGAGATTTGCAAGTCACAGTACTCACTGGAATGTATATAAAAT |
| | | | | AACTTTAATGATAAAAGACAATGAAAATGATTTTTGTGCATTTAAAA |
| | | | | AGATGTTTTTAAGTGACTATTTTTTTTCTAGGGTTTATCTTTTCTATTC |
| | | | | CTTCTCTCTCTTTTAATATGTAAATGGAATTTACGTTATAGGTTGAAG |
| | | | | GTCTTATTAATCAGGATGACATCTTGAGCAAATTGGTTCACATTCTA |
| | | | | GTCTAGATGCTTCGAGGCCATGGAATGGAGCCATGTCCCACAGTTT |
| | | | | CTATGTCTTTAATCCTAGGGGTCACGTGCCAATCTTTTCACCTAGATT |
| | | | | ATACGCAACTAAAATTTCCTAAGAGTAAGAGAAACTTTAGCTTCTTA |
| | | | | GTAAATCAAAGTTATCTAAATTTGTTAGATTAAGAGCTAATATTTCTT |
| | | | | TTAGCTGTTTAACACGTTAACCTCAGTTCTTTTAGGTTTGAGAGCAA |
| | | | | CCTTTTGCACCTAGGAGTCCATGTCCTTCATTTTTCTGTCCATCAGGT |
| | | | | TTTTATTTTCAAGCTCTTTTGTAAAATCTGAATGAGCCCCTAGCCGCA |
| | | | | TTCATATCACCTTACTTATGGCATCCTTTAGCCTAAAATGTTTGAAGC |
| | | | | AATAGGGAATCCTCTACTTTGGATAGTACAATGTTGCACAATCTCTG |
| | | | | CTTGCTTTTATGTGTATGCAATTCTTAAGATTAGACCGTCTTTTTAAA |
| | | | | GAAGAAGAAACATTTACTAGCGAAGATATAAAAAATACAGAGGGT |
| | | | | CACTGAATCTTTATTAGTGGGCTTTTAGACAACAAACTAAGTGGACA |
| | | | | CATTAGGTTAAATGACATTCTTTATGCTTAGCAGATCTTAGACAATT |
| | | | | CCTATTCCTAACCAAATAACTATGTCTTGAGATAAGAGTCTATCCTTA |
| | | | | ATAACTACCAGATTGCTGTGGTGGTACAAAGTATCGTGTATTAACCT |
| | | | | GAAACATAATTGGCCACCAGATCACAAATGAAGCTCTTGTGAAAGA |
| | | | | AAAGGTAGATCCCATTTTACTCGGACCCTTTTCTTCCTATTGCAGCTT |
| | | | | GGATCTTTCTGATTGGAAACTCTCTTTGCTGCTATATGATGTTCTTTA |
| | | | | AGTTCTATGGGTATCTTAGCTCTACAACTTTATTTCCTAATTCAACAA |
| | | | | TTACCATTTCCCACCTGCTCCAAATATTATTACTTGAAGAATTTTGGC |
| | | | | TGCTTGTATAAGTATGTTTGGTCCTTGAACTACATTATGTCTCCCCCC |
| | | | | TTCCCGAGCATTTTCTGTTAAGGGTTTTACTTTTCTGATGAATTATCA |
| | | | | TGTCTGCTTCGGAGTTGAACTCACATCAATATCTTGAAGGGTTTTAT |
| | | | | CCTCTATTTTTTAGGGTGATGTTTATCCCGTGGAGCCGTTTGATGGT |
| | | | | TTCTCCCCTCCAATCTTAGATGCTCATTGGGGTATTTTGAATGAAGA |
| | | | | AGATGTAAGCTACTTTCCTTTCTCTTTTATTTCTCTCGGGTTGTTTATT |
| | | | | TTCTAGTTCAATTGTTTGCCTTTTGTGTTCATTTTCGATGGATACATA |
| | | | | GTTTTATCATTCTCCAGTTTCAAATTTTAATGGATGCAACAAAATGAC |
| | | | | TCTTTAAATTATGTGCACAGTTTCGATGGATACACAATTTTGTTTTCT |
| | | | | CTAAGTCTAGGGTTGTGCATTTATGCAAAGTTTCTATTTGATGTTTG |
| | | | | CTATTAAGGGTCAATTAGAAACAGTTTCTTTGTTTCTATAAGGATAA |
| | | | | GGTTGCATATGTGTGGCTCTCCAAACCCCACGTAGATGGGAGATTA |
| | | | | GGTGGGAGCCAATTTATAGCGTTGGGGCAATTGAAAGTTGTTGTCT |
| | | | | ATTGTTTGCTCTCCAAGGATTTTAATTGCTCAAGTTTGTGAATGATG |
| | | | | CATTTCATAGTTCTCATTAAGAATTTGACTTCAGAGTAGAAAACTCC |
| | | | | CTCCCATAAGGTTCTGCTATGGTTTGAGTTAGCTGTGCATTTATTTTA |
| | | | | ATTTGTAGGGAAGTTGATGTATCCTTCATACTTTTTGTTCATTGGTAT |
| | | | | CATGTATATAATTGAGAGCACCGAGTTAAATTTAGCCGACACATTCC |
| | | | | ATTTTTTGGGGACTTTTGGAAAAGAGAGGCGGATGGACTGCTTTCG |
| | | | | AGTAGAGTATAAGAAAGAGACTCTTTCTTTGAACTGATTTCTATCAA |
| | | | | GATCTTTATAAATTTTTCCTATTATCGCCCCTATGCCCTCTTTATATTT |
| | | | | TACGAAGTACTTAACTTTTATATCGATGGTTAGATTGTGGT |
| 1713 | Amaranthus | gDNA | 4818 | AGCAGTTTATATGTTTGTTGAACTGTATACTGTATAATGGCTGATTC |
| | rudis | Contig | | AAATGTGGAAGTTGATATGATCCTTTTAAGTTTTAAGGTGTAGGGA |
| | | | | GAAGTTGTTTCTTCAATTCAGTAATGATAGAGTATATAATATATGAT |
| | | | | CCTTGGTTTTCGACCATATCACATGTTCGAATCTAGCCTCATATATAA |
| | | | | CATATCTTGAGGCTTCCACCATCAGCTTAAACTTCGGTTGAGTTGGT |
| | | | | TCCCTGTGAACTTCTCGCCTTCGCTTCACCTTTATGCTTGGTTTCAGC |
| | | | | CTTGCAAAGTTGCTAGTCGTGGAGAGGGTTACTTCAGGAAGGCTGT |
| | | | | CAGCATATATCACTGGCACTGCATATCTTTTTGAATACAAAATCATTT |
| | | | | TTAAGGAAATGACTCTTCCATTTATGTGTACTTATTTATGTAAAGTTG |
| | | | | CAAATGATGTTTGCTACCAAGAGTCAACCGGAAAAAACTTCTTTGTT |
| | | | | ATCAATAACAAGGGTAATATTGCGTACATTCCCCCTAAATCCCAAAC |
| | | | | CCCAGCCTAGTTGGAAGCCACTGAATTGCAATAGTGTAATGGAAAT |
| | | | | GTTGTTCTATATATATTTGGTTATCTTAATGTATCTTGTTATCTACACC |
| | | | | TGCTTCTCAGGTAATAAAACAGATTCAAAATCTGATATTTCATGTAT |
| | | | | TTCATTTTGTGTATACAGAGTGATGCGTCACACAATTTCTGTCTTTGT |
| | | | | TGGCGATGAAAGTGGGATAATCAATAGGATTGCCGGCGTTATTTCT |
| | | | | AGAAGAGGATACAATATCGAGTCTCTGGCTGTTGGTTTAAACAAGG |
| | | | | ATAAGGCTCTTTTTACTATAGTAGTGTGTGGGACTGACAAGGTGTTA |
| | | | | CGTCAAGTCGTGGAACAGCTTAACAAGCTTGTTAGTGTCTTGAAGG |
| | | | | TAGTTAATTTCATTTGTTCATCCTTTTATTGGTAAATGGTGATAGTCA |
| | | | | TACTATGACATAGTGCTTGGTTCAGTACTGTACGGATTGCGTTCGAT |
| | | | | AAAATTACTTTACAAGTCTTATAAACCCATTTTCCAAACTAGCTTATA |
| | | | | CAAACAAAACATATCGTGAATTTTTTTTTACTTAAATAGAAATGTTAT |
| | | | | TGTTTAATATGGAGCCTTACGTATCAAGCAATATAATGGATCATCTT |
| | | | | ATATGCATATGTTTGTACAATGTACATGTTTTGTTGGATTGTCTAATT |
| | | | | GTCCTATATAACATCCTTAGACGATTACTGCATTGCTTGTAGGTTGA |
| | | | | AGATCTATCGAGAGAGCCGCAAGTGGAACGTGAACTGATGCTTTTA |
| | | | | AAGCTTAATTCTGATGCAAATTCCCACGCAGAGGTAGATTCACAAAT |
| | | | | TCAATCAATAATCTATGAAGGCTATATTTTCCATGTTTTAAGTTCTAA |
| | | | | TTTCCTCGTCTAGTTGTCAGTTAGAGTCTAGCAAGGAACAAAAGATA |
| | | | | GAGATGTTTTGCTTTAGCTTTTATCCATGATCAAAAAATATGCTATAC |
| | | | | TTCATATAAGGCCATGACTGTTACAACAACAACAACAACAATGCCA |
| | | | | GAGCCTTAGTCCCAAAAGTTCATAGATGTTAGATAACTCAAATTTAT |
| | | | | CTAGAAGTACATCGTGTTGTGTGAAGCTCTTATAAGAAGTTGCAGT |
| | | | | AGTGTTTTTGTGAGAAATCTAATACCAATTAACGTAAATCTTCAATTT |
| | | | | TTGATCTTCAGCTAATGTGGCTAGTGGACATCTTCAGGGCAAAAATT |
| | | | | GTGGATATCTCAGAAAGCTTGGTCACTGTTGAGGTTAGTGGTGTCT |
| | | | | TTGTTTGATTTCTCGCGTACGTCTTCTAAGCGTACCTTCCAGAAGTTG |
| | | | | ACCTGAGATCTGACCAGTCGATGTAAAATTTTTTTCTTAGTGGCATT |
| | | | | TTCTTTGGTGTTGCAGGTGACTGGAGATCCTGGAAAGTTGGCTGCT |
| | | | | GTTCTAAGAAATTTTAGCAAGTTTGGAATCAAAGAAATTGCAAGGA |
| | | | | CCGGAAAGGTTAGTCTTTTAGACATTTGCTGTAAGTTCAGTATAGAA |
| | | | | TTCGGAAAGGCGCAAAGAGTACATGTCAACTATTGTGGCTAGCCAG |
| | | | | GCAACTATTGTGCATTTCTGGTGAGATCAATGTGAAACAAGTTTAGT |
| | | | | TAACTACTTGTTTTGTTTTATATACTTAATATGATAGATTGCTCTAAG |
| | | | | GCGAGAAAAGATGGGTCAGACAGCTCCTTTTTGGAGATTCTCTGCT |
| | | | | GCTTCTTATCCAGATCTACAAGAAAAGGCTGTTGATGCTCTTGCCAG |
| | | | | GCCTATGAAACGGAGCGTCAATGGTGATTCTGGCTCATCTTCAAGT |
| | | | | GTAAGTGAAATTTTATCTTCTCGACAATTATCTTTTTCCTTTTCTCACC |
| | | | | TTGCATTGCCTATGTATACTTTTTACCTTGTATTGCCTATAGACTTAA |
| | | | | ACATGCTTTTATTTTTTGAAGGGTGATGTTTATCCGGTGGAACCTTA |
| | | | | TGATGTTTCGATGGTAAATCAAGTACTTGATGCTCACTGGGGCGTA |
| | | | | CTTTATGACGGTGATGTAAGTGCTCTATCACTACCTGAATTGTCTGT |
| | | | | CACTAGGTCGTCCAATGGTCCATGTGATGTCATGTTCAAATCTCTTA |
| | | | | GGCCAATGTTTCCTAATTTAGTTCCAAATCAATTGTCTGAATTCACG |
| | | | | ATTCACCCAAAATGGCCTAAATTTTTCGTTTTAATTTACAAATGACCG |
| | | | | AGTCATGACAAACATGTATTAAGGTCTGCTCTATGTTACTTGGACTC |
| | | | | GGGTACTGAGTCGGATACTGGTACGTGTCCAAGTGTCATATGCGTC |
| | | | | TCAATATTCAATTTTACGCCTAAAAGGAAGTGTCTAAGTGTCATACC |
| | | | | AATGTCCGAGCATCAATGATCGGTCACTGGTACGTGAAGCAAAATG |
| | | | | AAGAGTCTGAGTAATAGCGGTTCAATCTCATTTGGATTTTGGAGGG |
| | | | | TTAAGGTTTATGTTTTTGGGTTTTCAGATGTTCGTCTTTCTGTTATTC |
| | | | | ATGTTTAATTTTAAGAATGAAAGTTAGTTCAACTTTTCAATTTTTTGA |
| | | | | GCTGCACAGTACTATAAATTGGTTTTAGTCTGCTCTGCTAGTATCCT |
| | | | | GATTTGCTTCGTTCTTCATGCCAGTCAAGTGGTCTCCGGTCACATAC |
| | | | | GCTGTCCATGCTGGTAAATAATGCTCCCGGAGTTCTTAACACTGTTA |
| | | | | CAGGAGTAATTTCTCGTAGGGGTTATAACATTCAGGTCAGTGTGGA |
| | | | | CTCTGACTGCAGCAAGAAAGTATATAAAGCCGTTTGTGTTATGTAAT |
| | | | | GACAATCGTCCCCTTTTCATCCTCTTCAGAGTCTTGCCGTAGGCCCT |
| | | | | GCTGAAAAGGAGGGTCTTTCTCGTATCACAACTGTCATTCCTGGAA |
| | | | | ACGATGAGTCGATTGGAAAATTGGTTCAGCAATTTAACAAGTTAGT |
| | | | | AGACCTTCATGAGGTGAGCAACTATTTATACTTCTGATGTTTTTTTTT |
| | | | | TTTGAATAATTTGCAATTTACAAGTTACAACCATAAAGTTAAAACTT |
| | | | | CATGTTAAGCATGCACACCCGCGTAATATTTTTTTTTGGGCAAATTTC |
| | | | | AGCATAAAAGTTTTTTCGAAATGATGTTTAAGCTTGTTGTACTTCAA |
| | | | | TGACTCGATGAAGAGAATTTGCGTTTAAAGAGAGAGGGAGAATGT |
| | | | | TGGATACAAAGTGAGAGAATAGGTAGTTCTTTTGAATTGGAGGAG |
| | | | | AGTAGTGATTGATTTAGTCAAACACAATTAGTGACGGTCAACAACT |
| | | | | GCTCGGTGAGTTAAAAAAATTTAGTTGTTAATCGGGCTGAAATCGG |
| | | | | TGAGCTGGTAGCAATCAAATGCCCGAGCTCTCTTTCATGGGTCGTA |
| | | | | AGTGGTATAGGCCATGTCCCTTGTTTAGTGGGTCAAAGATTTATAGT |
| | | | | TCTTTGGTTTCAAATTATGATAAACTATATTAAGATGACCGACTAAA |
| | | | | ATCATGTGCACTTCTTATTTACCTGAAGTTGCTGATGATCTTTGCTTA |
| | | | | CTAAGGGTCAATCAAAAACAACATTTTTGTTATCAACTAAAAGGGG |
| | | | | AAGTTGTGTACATCTGACCCTTCAAACCCCGCTTAGATGAGAGCCAC |
| | | | | TTAATGTCAATAATGGCAATAGGATGTAATAAGTGGTATAGACTAC |
| | | | | ATAGTAATAACTTTATGGTTTTAAGATGCAAAGTATCCTTATCACTCC |
| | | | | AATATGTGCTAAGAGGCTCAAAACAGAAGTGGACGAGAAAATTAT |
| | | | | GACATTAAAAGTATACACAATTTTAGATCCGCGTTTATGACAAACTA |
| | | | | CTTGATTGATTCTTGTATTTCTGAAAGCCCAGTTCAATCTCCTTTGTA |
| | | | | ATATGTTGATGCTCGTTAAGTTACCTATTTTCTTTGTAATGCGTAAGC |
| | | | | CATGAAGTTTGAGGTGTTATCTTAGTCCAACAACACATCATGCTGTT |
| | | | | TATTTATAAGTATTATGAAGTACTATGAATGAAAGCAGGGTGAACT |
| | | | | CAGGCCCGGGGCTGCCTAGGCCATGGCCCGAGTCATTTGATTAAAA |
| | | | | ACGCTTACAACACAGAAAAATAGTAGTCGGGTTGGTTCAACATTCA |
| | | | | TGCAATAATTCCAAGATATGATAATCTAACTTCAGAATGGTTGCTGC |
| | | | | TGCAGATTCAGGACCTTACGCACCAGCCATTTTCAGAGCGAGAGCT |
| | | | | TATGTTGATCAAAATAGCTGCAAATACTACAGCCAGGAGAGATGTT |
| | | | | CTTGATATTGCTAATATTTTCCGTGCAAAAGCTGTGGATGTTTCGGA |
| | | | | TCACACGATAACATTACAGGTACTAAGCCGTAGGCACACAATTTTTG |
| | | | | CCATGTTCCTTGTTTTTTCCTAATATTTCCGACTTGGTCTTTTCCAGCT |
| | | | | CGCCAATATGTTGGGATTAAGGCTTTGGCATTATTGTGTTATTTTTG |
| | | | | CGTTTTTTCCAACTTCCTTATATTGGGATTAA |
| 1714 | Amaranthus | gDNA | 1687 | TTTTTTGAGCTGCACAGTACTATAAATTGGTTTTAGTCTGCTCTGCTA |
| | rudis | Contig | | GTATCCTGATTTGCTTCGTTCTTCATGCCAGTCAAGTGGTCTCCGGT |
| | | | | CACATACGCTGTCCATGCTGGTAAATAATGCTCCTGGAGTTCTGAAC |
| | | | | ACCGTTACAGGAGTAATTGCTCGTAGAGGTTATAACATTCAGGTCA |
| | | | | GTATGGACTCTGACTGCACCATGAAAGTATATCCCGTCTTTTGCATT |
| | | | | ATGTTAACTTGATTGTCCGTTTTCCCTTCTTCCCAGAGTCTTGCTGTG |
| | | | | GGCCCTGCTGAAAAGGAGGGTCTTTCTCGTATCACAACTGTCATTCC |
| | | | | TGGAAACGATGAGTCGATTGGAAAATTGGTTCAGCAATTTAACAAG |
| | | | | TTAGTAGACCTTCATGAGGTGAGCATCTATTTGTGTACATCGTAGGC |
| | | | | ATAATTGTCCAAATTAGTTAGTGTCATGTTTACTTATGTCGTTAATAT |
| | | | | TTAATACTATTTTGATATTTGTTTTTTCGACTATTATGATTGAAAGAT |
| | | | | TAGAATATCTTATTCCGGAAAGTTTGTTGTCGCAGATTCAGGACCTT |
| | | | | ACTCACCAGCCATTTGCAGAGCGAGAGCTTATGTTAATTAAAGTAG |
| | | | | CGGCAAATACTTCTGCCAGGAGAGACGTCCTTGATATTGCTAATATT |
| | | | | TTCCGTGCAAAACCTGTGGATGTTTCTGATCATACAATAACATTACA |
| | | | | GGTACTAAGCCGTAGGCACACAATTTTTGCCATGTTCCTTGTTTTTTC |
| | | | | CTAATATTTCCGACTTGGTCTTTTCCAGCTCGCCAATATGTTGGGATT |
| | | | | AAGGCTTTGGCATTATTGTGTTATTTTTGCGTTTTTTCCAACTTCCTT |
| | | | | ATATTGGGATTAAGGCTTTGGCGTTGTTGCATTGTTGACTGTTGTTG |
| | | | | TGTCTTTTCCAGCTTACGGGTGATTTACACAAAATGGTTGCGCTACA |
| | | | | AAGATTATTGGAGCCTTATGGCATTTGTGAGGTATGCGCCTATGTAT |
| | | | | TATTGTTTTTTCTATATAATCAGATTTTATTTATTTATTTTTGGTTCGA |
| | | | | AATCTGTTGATTGTTAGTTATTTTGCTTTTGAATATAAATGGATTATC |
| | | | | CTGTCTGTAGATTAAGAACAATCCTGCTAAATAAGAAAATACACACA |
| | | | | TAGACCATAGTGCAAAAACACAGTAAAGGTCGCCATCGCGGTTATA |
| | | | | GATATTGTGATGCGGTAATGGGGATGATGTTGTGTGGTTGTTATCA |
| | | | | TAATAATTCATAACGCCATATGTCGGCTGAAAACATGAGAAGCGAT |
| | | | | ATTCCTTGAAACAACCCAAACACGGTTTTCTTTACCTATAAGCGAAT |
| | | | | ATCGGTTCGATTTTTTGAAAAATTTTACAATGCTGCCCTGTTTTTGCA |
| | | | | CAATGGAGACACGAGCACGCCCACACACACATATATAGGGAAAGG |
| | | | | ATCATGTGAAAACAAAAAAGTGGACCATGAAAATGGATGAGTGAG |
| | | | | CAATAAACGTGAAGATATCGTGAAGTTATCCTTCAATACTATAAAAT |
| | | | | GGTTTTCACTAGATCATATATACTTCACATCTTATAATTGTTTTATAC |
| | | | | CCTTATGTACCTCATGTGCATATTATATGTGAAGTTCCAGATAATATT |
| | | | | TGCTATACCAAGGGTCAATCGGAAACAACCATTGTTAGTTTATCAAT |
| | | | | AAAAAGGATAAAATTGTGTACATCAGACCCTCTAANACC |
| 1715 | Amaranthus | gDNA | 1648 | TTTGGTTTATTGTATTTTAGTTGGGCCTTTATGTTTGTTAGTGGGCTC |
| | rudis | Contig | | TTGGGTTCTATATATAGGGATAGTCTATCATTGTAAGTCTTGTCTTG |
| | | | | TATTTTGGAATAAGAAATTCAAAAATAAGGAGGCTACACAACACTC |
| | | | | GAAGTTGTGTGGGTTTGTGGGAGTTTACACGGCACTCAAAGGTGTG |
| | | | | TAGTATATCTTGTGTACTGTTTTCTTTGTGTTCTTTGAATTGCTCTTG |
| | | | | GTGGAAGGCAGGAAGTATTACGTAATACTAATAATACTACTAATAA |
| | | | | TAATATTAATAGTAATATGCCTAAGTTTCTGATATATACAACAAAGT |
| | | | | ACCAAAATTGGATCTCAAACCCTTTCTATACACTAACTAATGTAAGA |
| | | | | GTGATAATCCCTAGTGCTGATTCGCCTTTTGAATTCGCGATTCATTC |
| | | | | AATTTTGCTTTTTTTGATTTGTGGTGGACAAGAAGATTAGTGAATCA |
| | | | | CGTGACACTGATTTCCCTTTCCTTCATCTTACTCATACCATTTTATCCT |
| | | | | CTTTCCTTCATCTTACTCATACCATTTTATCCTCTTTCCTTCGTCTCTCC |
| | | | | TTTGGAATTCACCTCTACTTATCTCTATTTCTATAGTTTTACTTTTTCC |
| | | | | CCTTAAGTATTTACTCCTACAAGCATTCCCTTATACATAAAGAACACT |
| | | | | ATTTACGACAACAATGCTTGATGAATAGTATTTGGTGCAGTAAATGT |
| | | | | AGCTGCCATTATAAAACACGGAGGAAATAGAATGGAAAGTTAGGG |
| | | | | ATAAAATGAAACTTACTATAAATTATAATACGATTAAAAAGGAAACT |
| | | | | GAGACTTTCTATTTGGATCACTTGCATATATGAGTAGATTAATTATT |
| | | | | GATGGTCTCACTCTGGAAATGTATTCTGATCTATTTTGAATGAGTCA |
| | | | | TTGAATGAGATTTTTATTCCGGATCCAGGTTAGAGATCTTACCCATT |
| | | | | ATCCATTTGCTGAGCGAGAGTTGATGTTGATAAAAGTAGCTGTGAA |
| | | | | TACTGCTGCACGTCGTGAGGTCCTAGACGTTGCCAGCATTTTTAGAG |
| | | | | CAAAAGCTGTAGATGTATCTGATCACACCATAACACTTGAGGTAAG |
| | | | | TTTTGGGGAGTTTTATTAATTTTTATTTATAAATGGTGTCCCGTTTTC |
| | | | | TTTCAATTCGAAAAGGTCAATGTTTATAAAAAGACAACCAACAGAAT |
| | | | | AGATGTAGCTCTGTTAAGTATTTGTATGACATTGTGCTCTTAAGATA |
| | | | | TGTAAGCTGTTGGATTGCCTTGCAACGTTCATATTTTTGTTGTACATC |
| | | | | ATTCTGCTTTTACTTTACGGAGCCTGATTTTGATTAATGGGGATTTTG |
| | | | | ACTCAGCTCACTGGAGATTTGAACAAGATGGTTGCTCTACAGAGAT |
| | | | | TGCTCGAACCGTATGGAATCTGTGAGGTAAGATAGTATTTTTGGAC |
| | | | | AATATAGAGTTTCCTGAATGGATAAAAGCATTATTGTTTTTATGTTT |
| | | | | GCTGTTTTCTAATTGATTTCCGCAATAGAAATGTCAAAATTATCCTAT |
| | | | | TGCAGGTCTTATTGTCATTGATGCTCCTTATTCCATAATGCAATCACC |
| | | | | ATTTGCTTATATTAAACACAATGTGTACATATCTTTTTGATTTCAGCC |
| | | | | ATAATAATCCAAAGTTTACAGAGAAAAAGATGTTTCGTTCACG |
| 1716 | Amaranthus | gDNA | 1125 | ATCATCCAAGGTATTTGAAACATACTTGGAGTAATTTCTTTCTCTTCC |
| | rudis | Contig | | AACATTATAATGTCGATTTATGTGATTTGTGCTAAAGTGACACTTCA |
| | | | | TGTACCCTATCTTCCATCGACTTAATTTAAACTATCGTGACTTCAACT |
| | | | | CTTGTCTTCATCGTTATAACTTAGCTTCTACCCATTCCGCGACTTCAT |
| | | | | TTTCATCTACCTTGATATACCTTGATATAGCTTATCATTAATATTTTTC |
| | | | | TCATAATAATTCAAAAGTTTGCATCTATCTAATTTTAGTGCTACAAGT |
| | | | | GTTGCTTGTAGTAATTCTGTCTTGTTAGATTGTGGTGTGTGAACTAT |
| | | | | GTATTTTTCGTGAATTGTTGCATTTCTTGAATGACTTGTCTCTAGAAT |
| | | | | GATGCTCCTTAAACTTACTTCTTTTGTTTCATGTATGTTTAAGGGAGA |
| | | | | GACGACATACAATATCAGTATTTGTGGGGGATGAAAGTGGAATGAT |
| | | | | TAATCGAATAGCAGGGGTTTTTGCCAGAAGAGGTTATAATATCGAA |
| | | | | TCACTCGCTGTTGGATTGAACAAGGACAAAGCTCTCTTCACTATTGT |
| | | | | AGTTTCTGGAACGGATAATGTGTTGCAGCAAGTGATGGAACAGCTT |
| | | | | CAAAAGCTTGTCAATGTTTTGAAGGTTATTTTCCTTTAGAATAATTAC |
| | | | | CGTGGCATCCATGTCTTCAATTGTTTAGAATTCTCATCACTAAGTACA |
| | | | | ACTTATACATATAGGTTGAAGATATATCCAAGGAGCCTCAAGTAGA |
| | | | | ACGTGAATTGATGCTTGTAAAAGTTGGAGCTGATCGGAATAACCGT |
| | | | | GCTGAGGTACCTAATAATATATGTCACAGTACCCAACTGTACAAAAT |
| | | | | AGTTGATCTTATTAGATACAGACAGATATATTAGCTAGATCTCTATG |
| | | | | TTTTTTGTGGTGACTATTGACTTTCTGCTACATGCTGATCTTTCTTAG |
| | | | | TTTTCTCCTAGTAAATTGTAATTTTTAATATATAATTTGTTATTCCACT |
| | | | | ATGTCTGTTCCTGATTTACTTTCACTGATCATATTGTTCGTGTTTCTTC |
| | | | | TATTGTAGCTGATGTGGTTGGTGGACATCTTTCGTGCCAAAATTGTG |
| | | | | GACATATCGGAAGAGTATCTTTCAATAGAGGTAA |
| 1717 | Amaranthus | gDNA | 1029 | CTTTCTAGTCTCTTCCCCCCTCCCACCCTTCAATAGCTTATCCTTTCAT |
| | rudis | Contig | | GCCGTCCAACAAAATTTGAACTCATATCCTTGTGTATGATCGGAGAA |
| | | | | AGTAATGATTGAATCATTAATGATGCTCATCCTTGGTGAATTTATCT |
| | | | | CTTTTTATCCAGTCTTTGAGTCTCCTGGTTCAGGAACGCTGCCTATTA |
| | | | | ATCATTCCAAATCTCTTGAATAAAAGTCATTTTAAAATTATCATATGT |
| | | | | ATATCATTCCGTGCTGTTATTTTTCTTACATGTATTTTGCTATCATTAG |
| | | | | CGTGATAAAAAAATGTATTTTCATTCATTTCCAGGGTGATGCGCCAC |
| | | | | ACAATTTCTGTCTTTGTTGGGGATGAAAGTGGTATAATCAATAGGAT |
| | | | | TGCCGGCGTTATTTCTAGGAGAGGATACAACATAGAGTCTCTGGCT |
| | | | | GTTGGTTTAAATAAGGATAAGGCTCTTTTTACTATAGTAGTGTGTGG |
| | | | | GAATGACAAGGTGTTGCGCCAAGTAATGGAGCAGCTCAGCAAGCT |
| | | | | TGTTAGTGTCTTAAAGGTAGACATCACCTTTTTTCTTCCTTTAAAGTT |
| | | | | TAAGGTAACTAGTGATACTTTCATTTAGTAGGGGCGGTCAATGGTG |
| | | | | ATATTGATGCTAACCATGTATGTTCATTTTGTTTATGAAATATTGCTC |
| | | | | ATTTTGACTTTATCATTGTTGTTAAACTCTGAGAAAATGTAATCTCTA |
| | | | | AAGCATCTATCCTCACTTTAAGATAGATAGAGCAGGGAAACACTCA |
| | | | | TGATTCTTTTAATAAACTTTCAAAATATTAAAATTCCCTTTCCCTAAT |
| | | | | GCAGTTTTATAGCTCCCAGCTAATCTGAGTTCAAGGGGTTTGAGTGT |
| | | | | ATGCAAGCATACTCGTATAACAACAAAATGGTTGTCTCGGATTGAC |
| | | | | CCTTAAAATCAAATACCATCTTGCAAATTCACTTAAATACATACTTCA |
| | | | | ACGAACTGTTTTATAGTAGTCCATTAAAATTCGTAAGCAAAGAATTA |
| | | | | TAAAATTTTTTATTCCGTCGAGAATAGACACAATAAA |
| 1718 | Amaranthus | gDNA | 713 | TATTCTGATGTACTTTCGACATGCCAGTCAAGCGGACTTATGTCACA |
| | rudis | Contig | | TACTCTGTCCATGCTGGTAAATAATGCTCCTGGAGTTCTGAACACCG |
| | | | | TTACAGGAGTAATTGCTCGTAGAGGTTATAACATTCAGGTCAGTAT |
| | | | | GGACTCTGACTGCACCATGAAAGTATATCCCGTCTTTTGCATTATGT |
| | | | | TAACTTGATTGTCCCTTTTTCCTTCTTCCTAGAGTCTTGCTGTGGGCC |
| | | | | CTGCTGAAAAGGAGGGTCTTTCTCGTATCACCACCGTTGTTCCTGGA |
| | | | | AACGACGAGTCAATTGCAAAATTGGTTCAGCAATTAAACAAATTAG |
| | | | | TAGACCTTCATGAGGTGAGCATCTATTTGTGTACATCGTAAGCATAA |
| | | | | TTGTCCAAATTAGTTAGTGTCATGTTTACTTATGTCGTTAATATTTAA |
| | | | | TACTATTTTGATATTTGTTTTTTCGACTATTATGATTGAAAGATTAGA |
| | | | | ATATCTTATTCCGGAAAGGTTGTGGTCGCAGATTCAGGACCTTACTC |
| | | | | ACCAGCCATTTGCAGAGCGAGAGCTTATGTTAATTAAAGTAGCGGC |
| | | | | AAATACTTCTGCCAGGAGAGACGTCCTTGATATTGCTAATATTTTCC |
| | | | | GTGCAAAACCTGTGGATGTTTCTGATCATACAATAACATTACAAGTA |
| | | | | CGCATAAATGATTTTGCCATTTTATTTAGCCTTCTCTTGTTACTCTCA |
| | | | | ACAATGT |
| 1719 | Amaranthus | cDNA | 1392 | CTACCATCTCCATTCTCCCCATTTCATTTCTTTCTCATAACTCTCTAAT |
| | spinosus | Contig | | GGCGGCCATTTCCTTTAACATCAATGGCGGAAAGATCGGAACTTTA |
| | | | | TGTCCAAAACCTAAATATGGTTGTGGGTTTTTGAGAAAATGGGATTT |
| | | | | TGGAGCTCATACAACTGTATCTACTAAACCCATGTCAAAAATTTTAA |
| | | | | GCTTGAAAGCAGTTGAAGTTTCTGCTGATGCTACAGTAAATGCAGT |
| | | | | TTCTGTTTCATCTAATTCTAGGGTGATGCGCCACACAATTTCAGTCTT |
| | | | | TGTCGGGGATGAAAGTGGTATAATCAATAGGATTGCAGGCGTTATT |
| | | | | TCTAGAAGAGGATACAATATCGAGTCTTTGGCTGTTTGTTTAAACAA |
| | | | | GGATAAGGCTCTTTTTACTATAGTAGTGTGTGGAACTGACAAGGTG |
| | | | | TTGCGCCAAGTAATGGAGCAGCTCAGCAAGCTTGTTAATGTCTTAA |
| | | | | AGGTTGAAGATCTATCTAGAGAGCCTCAAGTGGAACGTGAGCTTAT |
| | | | | GCTATTAAAGCTTCATTCTAATGCAGATACCCATGCAGAGATAATGT |
| | | | | GGTTAGTGGACATCTTCAGAGCAAAAATTGTCGATATGTCGGAAAG |
| | | | | CTTCGTTACTGTAGAGGTGACTGGTGATCCTGGAAAGATGGCTGCT |
| | | | | GTCCTGAGAAATTTTAGCAAGTATGGAATCAAAGAAGTTGCAAGAA |
| | | | | CAGGAAAGATTGCTCTAAGACGGGAAAGGATGGGCCAGACAGCTC |
| | | | | CTTTTTGGAGATTCTCTGCTGCATCTTATCCAGATCTAAAAGAAAAG |
| | | | | GCTGTTGAATCTTTTGTCAGGCCTGCCAAAAGAAACATCAACGCTG |
| | | | | ATCCTGGCTCATCGTCTAGTGGTGATGTTTATCCAGTGGAGCCTTAT |
| | | | | GAAGCCTCCATAAATACAGTACTTGATGCTCACTGGGGCGTTCTTTA |
| | | | | CGAAAATGATTCAAGCGGACTTGTATCACATACTCTGTCCATGCTGG |
| | | | | TAAATAATGCTCCTGGAGTTCTGAACACCGTTACAGGAGTAATTGCT |
| | | | | CGTAGAGGTTATAACATTCAGAGTCTTGCTGTGGGCCCTGCTGAAA |
| | | | | AGGAGGGTCTTTCTCGTATCACAACTGTTGTTCCTGGAAACGACGA |
| | | | | GTCAATTGCAAAATTGGTTCAGCAATTAACAAATTAGTAGACCTTCA |
| | | | | TGAGATTCAGGACCTTACGCACCAGCCATTTGCAGAGCGAGAGCTT |
| | | | | ATGTTAATTAAAGTAGCTGCAAATACTACAGCCAGGAGAGATGTCC |
| | | | | TTGATATTGCTAATATTTTCCGTGCAAAAGCTGTGGATGTTTCGGAT |
| | | | | CACACGATAACATTACAGCTTACGGGTGATTTACACAAAATGGTTG |
| | | | | CGCTACAGAGACTATTGGAGCCTTATGGCATTTGTGAGGTGGC |
| 1720 | Amaranthus | cDNA | 579 | CTACCATCTCCATTCTCCCCATTTCATTTCTTTCTCATAACTCTCTAAT |
| | spinosus | Contig | | GGCGGCCATTTCCTTTAACATCAATGGCGGAAAGATCGGAACTTTA |
| | | | | TGTCCAAAACCTAAATATGGTTGTGGGTTTTTGAGAAAATGGGATTT |
| | | | | TGGAGCTCATACAACTGTATCTACTAAACCCATGTCAAAAATTTTAA |
| | | | | GCTTGAAAGCAGTTGAAGTTTCTGCTGATGCTACAGTAAATGCAGT |
| | | | | TTCTGTTTCATCTAATTCTAGGGTGATGCGCCACACAATTTCAGTCTT |
| | | | | TGTCGGGGATGAAAGTGGTATAATCAATAGGATTGCAGGCGTTATT |
| | | | | TCTAGAAGAGGATACAATATCGAGTCTTTGGCTGTTTGTTTAAACAA |
| | | | | GGATAAGGCTCTTTTTACTATAGTAGTGTGTGGAACTGACAAGGTG |
| | | | | TTGCGCCAAGTCGTGGAACAGCTTAACAAGCTTGTTAGTGTTTTGAA |
| | | | | GGTTGAAGATCTATCGAGAGAGCCACAAGTGGAACGTGAACTGAT |
| | | | | GCTTGTAAAGCTTAATTCTGATTCAAATTCCCACGCAGAGCTAATGT |
| | | | | GGCTAGTGGACATCTTCA |
| 1721 | Amaranthus | cDNA | 497 | CTCATCAGGCCGAGTTAGCTGTGTTTCAAATTTCCATCCATGGAGGC |
| | spinosus | Contig | | TGTGTCGACTCACCTTTCAACGAGTTTTAACTCCATTCCGAAAAGCA |
| | | | | ATAGATTGAGCCACCAAACTGCAAAACGATTAGGGTTCTCCTTGAA |
| | | | | ACCCCATTCTCTGGGTTTTAAGTTTAACTCCAATAGTGACAGGAATT |
| | | | | CGGAGTTTGATAAACTGGTTGTATCTGCAAGCAATGTTGATCAACT |
| | | | | GGGAAATCAAAGTAACTTATCCTTTAATCCCCCTTCTCCCTCTCGATC |
| | | | | AAAGGAGAGACGACATACAATATCAGTATTTGTGGGGGATGAAAG |
| | | | | TGGAATGATTAATCGAATAGCAGGGGTTTTGCCAGAAGAGGTTATA |
| | | | | ATATCGAATCACTCGCTGTTGGATTGAACAAGGACAAAGCTCTCTTC |
| | | | | ACTATTGTAGTTTCCGGAACGGATAATGTGCTGCAGCAAGTGATGG |
| | | | | AACAGCTTCAAAAGCTTGTCAATGTTTTGAAG |
| 1722 | Amaranthus | cDNA | 495 | TCTCTAATGGCGGCTGTTTCCTTCAATATTAATGGTGGAAAGATTGG |
| | thunbergii | Contig | | AATTTTATGTTCAAGACACGAATTCGGTTGTGGGTTTGTAAGAAAAT |
| | | | | TGGATTTTAGAACTCATACTTCTATATTTGAAAAACATATGCCAAAA |
| | | | | ACTTCAAGTTTTAAAGCAATGGAAGTTTCTGCAAATGCAACAGTAA |
| | | | | ATATAGTTCCAGTTTCAGCTCATTCTAGGGTGATGCGCCACACAATT |
| | | | | TCAGTCTTTGTTGGGGATGAAAGTGGTATAATCAATAGGATTGCCG |
| | | | | GCGTTATTTCTAGGAGAGGATACAACATAGAGTCTCTTGCTGTTGGT |
| | | | | TTAAATAAGGATAAGGCTCTTTTTACTATAGTAGTGTGTGGAACTGA |
| | | | | CAAGGTGTTGCGCCAAGTAATGGAGCAGCTCAGCAAGCTTGTTAAT |
| | | | | GTCTTAAAGGTTGAAGATCTATCTAGAGAGCCTCAAGTGGAACGTG |
| | | | | AACTTATGCTATTAAAGCTTCATTCTAAT |
| 1723 | Amaranthus | cDNA | 1729 | CTACCATCTCCATTCTCCCCATTTCATTTCTTTCTCATCTCTCTCCAAT |
| | viridis | Contig | | GGCGGCCATTTCCTTTAACATCAATGGCGGAAAGATTGGAACTTTAT |
| | | | | GTCCAAAACCTAAATATGGTTGTGGGTTTTTGAGAAAATGGGATTTT |
| | | | | GGAGCTCATACAACTGTATATACTAAACCCATGTCAAAAATTTCAAG |
| | | | | CCTGAAAGCAGTTGAAGTTTCTACCAATGCTACAGTAAAATGCAGTT |
| | | | | TCTGTTTCAGCTAATTCTAGGGTGATGCGATCACACAATTTCTGTCTT |
| | | | | TGTCGGGGATGAAAGTGGATAATCAATAGGATTGCAGGTGTTATTT |
| | | | | CTAGAAGAGGATACAATATCGAGTCTTTGGCTGTTTGTTTAAACAA |
| | | | | GGATAAGGCTCTTTTTACTATAGAAGTGTGTGGAACTGACAAGGTG |
| | | | | TTGCGCCAAGTCGTGGAACAGCTTAACAAGCTTGTTAGTGTTTTGAA |
| | | | | GGTTGAAGATCTATCGAGAGAGCCACAAGTGGAACGTGAACTGAT |
| | | | | GCTTGTAAAGCTTAATTCTGATGCAATTCCCACGCAGAGCTAATGTG |
| | | | | GCTAGTGGACATCTTCAGGGCAAAAATTGTGGATATCTCAGAAAGC |
| | | | | TTGGTCACTGTTGAGGTGACCGGAGATCCTGGAAAGTTGGCTGCTG |
| | | | | TCCTAAGAAATTTTAGAAAGTTTGGAATCAAAGAAATTGCAAGGAC |
| | | | | CGGTAAGATTGCTCTAAGACGAGAAAGGATGGGCCAGACAGCTCC |
| | | | | TTTTTGGAGATTCTCTGCTGCATCTTATCCAGATCTACAAGAAAAGG |
| | | | | CTGTTGATGATCTGGCCAGGCCTACGAAACGGAGCATCAATGGTGA |
| | | | | TTCTGGCTCATCTTCAAGTGGTGATGTTTATCCGGTGGAACCTTATG |
| | | | | ATGGTTCGATGGTAAATCAAGTACTTGATGCTCACTGGGGCGTACT |
| | | | | TTATGACGGTGATTCAAGTGGTCTCCGGTCACATACTCTGTCCATGC |
| | | | | TGGTAAATAATGCTCCTGGAGTTCTGAACACCGTTACAGGAGTAAT |
| | | | | TGCTCGTAGAGGTTATAACATTCAGAGTCTTGCTGTGGGCCCTGCT |
| | | | | GAAAAGGAGGGTCTTTCTCGTATCACAACTGTTGTTCCTGGAAACG |
| | | | | ACGAGTCAATTGCAAAATTGGTTCAGCAATTAAACAAATTAGTAGA |
| | | | | CCTTCATGAGATTCAGGACCTTACGCACCAGCCATTTGCAGAGCGA |
| | | | | GAGCTTATGTTAATTAAAGTAGCGGCAAATACTTCTGCCAGGAGAG |
| | | | | ACGTCCTTGATATTGCTAATATTTTCCGTGCGAAACCTGTGGATGTT |
| | | | | TCTGATCACACAATAACATTACAACTTGCTGGTGATTTAGACAAAAT |
| | | | | GGTTGCGCTACAGAGATTATTGGAGCCTTACGGCATTTGTGAGGTG |
| | | | | GCACGGACTGGAAGAGTAGCACTAACGAGAGAGTCTAGGGTAGAT |
| | | | | TCCAAATATCTACGAGGATACACTCTTCCGTTGTATGAATGAAAAAC |
| | | | | TCGGGCTATGTCTTTCCGACATTACCTTGTCTGATTCCTTCCGCTCTT |
| | | | | CTACACTGCATTGCAAGCAGAACAATTGCCCACAAGTGGAGTAAAT |
| | | | | TAAAAGGGGAAACAACAATACCGATCTTTGCTTTGAAGATATACTG |
| | | | | ATTTGTATAACAATAGAGTTTGTATTTGACTAGTATTTATTTGCTCAA |
| | | | | CGCTGTAATAACATATCCCCTTGAGGTTTGATATATGGAAGGAAAA |
| | | | | ATTACCCAG |
| 1724 | Amaranthus | cDNA | 1726 | CTACCATCTCCATTCTCCCCATTTCATTTCTTTCTCATCTCTCTCCAAT |
| | viridis | Contig | | GGCGGCCATTTCCTTTAACATCAATGGCGGAAAGATTGGAACTTTAT |
| | | | | GTCCAAAACCTAAATATGGTTGTGGGTTTTTGAGAAAATGGGATTTT |
| | | | | GGAGCTCATACAACTGTATATACTAAACCCATGTCAAAAATTTCAAG |
| | | | | CCTGAAAGCAGTTGAAGTTTCTACCAATGCTACAGTAAAATGCAGTT |
| | | | | TCTGTTTCAGCTAATTCTAGGGTGATGCGATCACACAATTTCTGTCTT |
| | | | | TGTCGGGGATGAAAGTGGATAATCAATAGGATTGCAGGTGTTATTT |
| | | | | CTAGAAGAGGATACAATATCGAGTCTTTGGCTGTTTGTTTAAACAA |
| | | | | GGATAAGGCTCTTTTTACTATAGAAGTGTGTGGAACTGACAAGGTG |
| | | | | TTGCGCCAAGTCGTGGAACAGCTTAACAAGCTTGTTAGTGTTTTGAA |
| | | | | GGTTGAAGATCTATCGAGAGAGCCACAAGTGGAACGTGAACTGAT |
| | | | | GCTTGTAAAGCTTAATTCTGATGCAATTCCCACGCAGAGCTAATGTG |
| | | | | GCTAGTGGACATCTTCAGGGCAAAAATTGTGGATATCTCAGAAAGC |
| | | | | TTGGTCACTGTTGAGGTGACCGGAGATCCTGGAAAGTTGGCTGCTG |
| | | | | TCCTAAGAAATTTTAGAAAGTTTGGAATCAAAGAAATTGCAAGGAC |
| | | | | CGGTAAGATTGCTCTAAGACGAGAAAGGATGGGCCAGACAGCTCC |
| | | | | TTTTTGGAGATTCTCTGCTGCATCTTATCCAGATCTACAAGAAAAGG |
| | | | | CTGTTGAATCTTTTGTCAGGCCTGCCAAAAGAAACATCAACGCTGAT |
| | | | | CCTGGCTCATCGTCTAGTGGTGATGTTTATCCAGTGGAGCCTTATGA |
| | | | | AGCCTCCATAAATACAGTACTTGATGCTCACTGGGGCGTTCTTTACG |
| | | | | AAAATGATTCAAGCGGACTTGTATCACATACTCTGTCCATGCTGGTA |
| | | | | AATAATGCTCCTGGAGTTCTGAACACCGTTACAGGAGTAATTGCTC |
| | | | | GTAGAGGTTATAACATTCAGAGTCTTGCTGTGGGCCCTGCTGAAAA |
| | | | | GGAGGGTCTTTCTCGTATCACAACTGTTGTTCCTGGAAACGACGAG |
| | | | | TCAATTGCAAAATTGGTTCAGCAATTAAACAAATTAGTAGACCTTCA |
| | | | | TGAGATTCAGGACCTTACGCACCAGCCATTTGCAGAGCGAGAGCTT |
| | | | | ATGTTAATTAAAGTAGCGGCAAATACTTCTGCCAGGAGAGACGTCC |
| | | | | TTGATATTGCTAATATTTTCCGTGCGAAACCTGTGGATGTTTCTGAT |
| | | | | CACACAATAACATTACAACTTGCTGGTGATTTAGACAAAATGGTTGC |
| | | | | GCTACAGAGATTATTGGAGCCTTACGGCATTTGTGAGGTGGCACGG |
| | | | | ACTGGAAGAGTAGCACTAACGAGAGAGTCTAGGGTAGATTCCAAA |
| | | | | TATCTACGAGGATACACTCTTCCGTTGTATGAATGAAAAACTCGGGC |
| | | | | TATGTCTTTCCGACATTACCTTGTCTGATTCCTTCCGCTCTTCTACACT |
| | | | | GCATTGCAAGCAGAACAATTGCCCACAAGTGGAGTAAATTAAAAGG |
| | | | | GGAAACAACAATACCGATCTTTGCTTTGAAGATATACTGATTTGTAT |
| | | | | AACAATAGAGTTTGTATTTGACTAGTATTTATTTGCTCAACGCTGTA |
| | | | | ATAACATATCCCCTTGAGGTTTGATATATGGAAGGAAAAATTACCCAG |
| 1725 | Amaranthus | cDNA | 1565 | CTACCATCTCCATTCTCCCCATTTCATTTCTTTCTCATCTCTCTCCAAT |
| | viridis | Contig | | GGCGGCCATTTCCTTTAACATCAATGGCGGAAAGATTGGAACTTTAT |
| | | | | GTCCAAAACCTAAATATGGTTGTGGGTTTTTGAGAAAATGGGATTTT |
| | | | | GGAGCTCATACAACTGTATATACTAAACCCATGTCAAAAATTTCAAG |
| | | | | CCTGAAAGCAGTTGAAGTTTCTACCAATGCTACAGTAAAATGCAGTT |
| | | | | TCTGTTTCAGCTAATTCTAGGGTGATGCGATCACACAATTTCTGTCTT |
| | | | | TGTCGGGGATGAAAGTGGATAATCAATAGGATTGCAGGTGTTATTT |
| | | | | CTAGAAGAGGATACAATATCGAGTCTTTGGCTGTTTGTTTAAACAA |
| | | | | GGATAAGGCTCTTTTTACTATAGAAGTGTGTGGAACTGACAAGGTG |
| | | | | TTGCGCCAAGTCGTGGAACAGCTTAACAAGCTTGTTAGTGTTTTGAA |
| | | | | GGTTGAAGATCTATCGAGAGAGCCACAAGTGGAACGTGAACTGAT |
| | | | | GCTTGTAAAGCTTAATTCTGATGCAATTCCCACGCAGAGCTAATGTG |
| | | | | GCTAGTGGACATCTTCAGGGCAAAAATTGTGGATATCTCAGAAAGC |
| | | | | TTGGTCACTGTTGAGGTGACCGGAGATCCTGGAAAGTTGGCTGCTG |
| | | | | TCCTAAGAAATTTTAGAAAGTTTGGAATCAAAGAAATTGCAAGGAC |
| | | | | CGGTAAGATTGCTCTAAGACGAGAAAGGATGGGCCAGACAGCTCC |
| | | | | TTTTTGGAGATTCTCTGCTGCATCTTATCCAGATCTACAAGAAAAGG |
| | | | | CTGTTGATGATCTGGCCAGGCCTACGAAACGGAGCATCAATGGTGA |
| | | | | TTCTGGCTCATCTTCAAGTGGTGATGTTTATCCGGTGGAACCTTATG |
| | | | | ATGGTTCGATGGTAAATCAAGTACTTGATGCTCACTGGGGCGTACT |
| | | | | TTATGACGGTGATTCAAGTGGTCTCCGGTCACATACTCTGTCCATGC |
| | | | | TGGTAAATAATGCTCCTGGAGTTCTGAACACCGTTACAGGAGTAAT |
| | | | | TTCTCGTAGGGGTTATAACATTCAGAGTCTTGCTGTAGGCCCTGCTG |
| | | | | AAAAGGAGGGTCTTTCTCGTATCACAACTGTCATTCCTGGAAACGA |
| | | | | CGAGTCGATTGGAAAATTGGTTCAGCAATTCAACAAGTTAGTAGAC |
| | | | | CTTCATGAGATTCAGGACCTTACTCACCAGCCATTTTCAGAGCGAGA |
| | | | | GCTTATGTTGATCAAAATAGCTGCAAATACTACAGCCAGGAGAGAT |
| | | | | GTCCTTGATATTGCTAATATTTTCCGTGCAAAAGCTGTGGATGTTTC |
| | | | | GGATCACACGATAACATTACAGCTTGCGGGTGATTTACACAAAATG |
| | | | | GTTGCGCTACAAAGATTATTGGAGCCTTATGGCATTTGTGAGGTGG |
| | | | | CACGGACAGGAAGAGTCGCACTGAGTAGAGAGTCTGGGGTCGATT |
| | | | | CTACATCTTTACGTGGATACGCTCTTCCTTTGTACGAATAAAAATTCT |
| | | | | CCGGCTATGTCTTTTCGTCATTATTATGTCCCATTTCCTTCAGCACGT |
| | | | | CTGCGCTACATTTACATAGCGATCAATTG |
| 1726 | Amaranthus | cDNA | 1562 | CTACCATCTCCATTCTCCCCATTTCATTTCTTTCTCATCTCTCTCCAAT |
| | viridis | Contig | | GGCGGCCATTTCCTTTAACATCAATGGCGGAAAGATTGGAACTTTAT |
| | | | | GTCCAAAACCTAAATATGGTTGTGGGTTTTTGAGAAAATGGGATTTT |
| | | | | GGAGCTCATACAACTGTATATACTAAACCCATGTCAAAAATTTCAAG |
| | | | | CCTGAAAGCAGTTGAAGTTTCTACCAATGCTACAGTAAAATGCAGTT |
| | | | | TCTGTTTCAGCTAATTCTAGGGTGATGCGATCACACAATTTCTGTCTT |
| | | | | TGTCGGGGATGAAAGTGGATAATCAATAGGATTGCAGGTGTTATTT |
| | | | | CTAGAAGAGGATACAATATCGAGTCTTTGGCTGTTTGTTTAAACAA |
| | | | | GGATAAGGCTCTTTTTACTATAGAAGTGTGTGGAACTGACAAGGTG |
| | | | | TTGCGCCAAGTCGTGGAACAGCTTAACAAGCTTGTTAGTGTTTTGAA |
| | | | | GGTTGAAGATCTATCGAGAGAGCCACAAGTGGAACGTGAACTGAT |
| | | | | GCTTGTAAAGCTTAATTCTGATGCAATTCCCACGCAGAGCTAATGTG |
| | | | | GCTAGTGGACATCTTCAGGGCAAAAATTGTGGATATCTCAGAAAGC |
| | | | | TTGGTCACTGTTGAGGTGACCGGAGATCCTGGAAAGTTGGCTGCTG |
| | | | | TCCTAAGAAATTTTAGAAAGTTTGGAATCAAAGAAATTGCAAGGAC |
| | | | | CGGTAAGATTGCTCTAAGACGAGAAAGGATGGGCCAGACAGCTCC |
| | | | | TTTTTGGAGATTCTCTGCTGCATCTTATCCAGATCTACAAGAAAAGG |
| | | | | CTGTTGAATCTTTTGTCAGGCCTGCCAAAAGAAACATCAACGCTGAT |
| | | | | CCTGGCTCATCGTCTAGTGGTGATGTTTATCCAGTGGAGCCTTATGA |
| | | | | AGCCTCCATAAATACAGTACTTGATGCTCACTGGGGCGTTCTTTACG |
| | | | | AAAATGATTCAAGCGGACTTGTATCACATACTCTGTCCATGCTGGTA |
| | | | | AATAATGCTCCTGGAGTTCTGAACACCGTTACAGGAGTAATTTCTCG |
| | | | | TAGGGGTTATAACATTCAGAGTCTTGCTGTAGGCCCTGCTGAAAAG |
| | | | | GAGGGTCTTTCTCGTATCACAACTGTCATTCCTGGAAACGACGAGTC |
| | | | | GATTGGAAAATTGGTTCAGCAATTCAACAAGTTAGTAGACCTTCAT |
| | | | | GAGATTCAGGACCTTACTCACCAGCCATTTTCAGAGCGAGAGCTTAT |
| | | | | GTTGATCAAAATAGCTGCAAATACTACAGCCAGGAGAGATGTCCTT |
| | | | | GATATTGCTAATATTTTCCGTGCAAAAGCTGTGGATGTTTCGGATCA |
| | | | | CACGATAACATTACAGCTTGCGGGTGATTTACACAAAATGGTTGCG |
| | | | | CTACAAAGATTATTGGAGCCTTATGGCATTTGTGAGGTGGCACGGA |
| | | | | CAGGAAGAGTCGCACTGAGTAGAGAGTCTGGGGTCGATTCTACATC |
| | | | | TTTACGTGGATACGCTCTTCCTTTGTACGAATAAAAATTCTCCGGCT |
| | | | | ATGTCTTTTCGTCATTATTATGTCCCATTTCCTTCAGCACGTCTGCGC |
| | | | | TACATTTACATAGCGATCAATTG |
| 1727 | Amaranthus | cDNA | 1441 | GTCTTTCTTTGCACCCTGGTTGTCCTCGTCAGGCCGAGTTAGCTGTG |
| | viridis | Contig | | TTTCAAATTTCCATCCATGGAGGCTGTGTCGACTCACCTTTCAACGA |
| | | | | GTTTTAACTCCATTCCGAAAAGCAATAGATTGAACCACCAAACTGCA |
| | | | | AAACGATTAGGGTTCTCCTTGAAACCCCATTCTCTGGGTTTTAAGTT |
| | | | | TAACTCCAATAGTGACAGGAATTCGGAGTTTGATAAACTGGTTGTA |
| | | | | TCTGCAAGCAATGTTGATCAACTGGGAAATCAAAGTAACTTATCCTT |
| | | | | TAATCCCCCTTCTCCCTCTCGATCAAAGGAGAGACGACATACAATAT |
| | | | | CAGTATTTGTGGGGGATGAAAGTGGAATGATTAATCGAATAGCAG |
| | | | | GGGTTTTTGCCAGAAGAGGTTATAATATCGAATCACTCGCTGTTGG |
| | | | | ATTGAACAAGGACAAAGCTCTCTTCACTATTGTAGTTTCTGGAACGG |
| | | | | ATAATGTGTTGCAGCAAGTGATGGAACAGCTTCAAAAGCTTGTCAA |
| | | | | TGTTTTGAAGGTTGAAGATATATCCAAGGAGCCTCAAGTAGAACGT |
| | | | | GAATTGATGCTTGTAAAAGTTGGAGCTGATCGGAATAACCGTGCTG |
| | | | | AGCTGATGTGGTTGGTGGACATCTTTCGTGCCAAAATTGTGGACAT |
| | | | | ATCGGAAGAGTATCTTTCAATAGAGGTCACTGGAGATCCAGGAAAG |
| | | | | ATGGTTGCTGTCCTTAGAAACCTAAGCAAGTTTGGCATCAAAGAAA |
| | | | | TTGCTCGTACCGGAAAGATTGCTCTAAGAAGGGAAAAATTGGGCG |
| | | | | AGTCTGCTCCTTTCTGGCGTTTTTCTGCTGCTTCTTATCCTGATCTTG |
| | | | | AAGAAGCTATCCCTATGGATGCTCTTTCTGGAGTTTCAAAAGGGGC |
| | | | | AGCTGCTGCTGGATCATCGGATTCGTCTGTGGAGGGTGATGTTTAT |
| | | | | CCTGTGGAGCCGTTTGATGGTTTCTCCCTTCCAATCTTAGATGCTCAT |
| | | | | TGGGGTATTTTGAACGAAGAAGATACTAGTGGGATGCGGTCACAC |
| | | | | ACTCTATCTATTCTTGTTAATGACAAACCTGGGGTCCTTAATGTTGTT |
| | | | | ACGGGGGTTTTTGCTCGAAGGGGTTATAACATTCAGAGTTTAGCTG |
| | | | | TGGGTCATGCGGAAGGTGAGGGTCTATCTCGTATCACTACTGTTGT |
| | | | | ACCCGGTACAGATGAATCAATTAGCAAATTGGTTCAACAAATCTAC |
| | | | | AAGCTGGTTGATATTCATGAGGTTAGAGATCTTACCCATTATCCATT |
| | | | | TGCTGAGCGAGAGTTGATGTTGATAAAAGTAGCTGTGAATACTGCT |
| | | | | GCACGTCGTGAGGTCCTAGACGTTGCCAGCATTTTTAGAGCAAAAG |
| | | | | CTGTTGATGTATCTGATCACACCATAACACTTGAGCTCACTGGAGAT |
| | | | | TTGAACAAGATGGTTGCTCTACAGAGATTGCTCGAACCGTATGGAA |
| | | | | TCT |
| 1728 | Amaranthus | cDNA | 1257 | GAAGATCTATCTAGAGAGCCTCAAGTGGAACGTTGAGCTTATGCTA |
| | viridis | Contig | | TTAAAGCTTCATTCTAATGCAGATACCCATGCAGAGATAATGTGGTT |
| | | | | AGTGGACATCTTCAGAGCAAAAATTGTCGATATGTCGGAAAGCTTC |
| | | | | GTTACTGTAGAGGTGACTGGTGATCCTGGAAAGATGGCTGCTGTCC |
| | | | | TGAGAAATTTTAGCAAGTATGGAATCAAAGAAGTTGCAAGAACAG |
| | | | | GAAAGATTGCTCTAAGACGAGAAAGGATGGGCCAGACAGCTCCTTT |
| | | | | TTGGAGATTCTCTGCTGCATCTTATCCAGATCTACAAGAAAAGGCTG |
| | | | | TTGATGATCTGGCCAGGCCTACGAAACGGAGCATCAATGGTGATTC |
| | | | | TGGCTCATCTTCAAGTGGTGATGTTTATCCGGTGGAACCTTATGATG |
| | | | | GTTCGATGGTAAATCAAGTACTTGATGCTCACTGGGGCGTACTTTAT |
| | | | | GACGGTGATTCAAGTGGTCTCCGGTCACATACTCTGTCCATGCTGGT |
| | | | | AAATAATGCTCCTGGAGTTCTGAACACCGTTACAGGAGTAATTGCTC |
| | | | | GTAGAGGTTATAACATTCAGAGTCTTGCTGTGGGCCCTGCTGAAAA |
| | | | | GGAGGGTCTTTCTCGTATCACAACTGTTGTTCCTGGAAACGACGAG |
| | | | | TCAATTGCAAAATTGGTTCAGCAATTAAACAAATTAGTAGACCTTCA |
| | | | | TGAGATTCAGGACCTTACGCACCAGCCATTTGCAGAGCGAGAGCTT |
| | | | | ATGTTAATTAAAGTAGCGGCAAATACTTCTGCCAGGAGAGACGTCC |
| | | | | TTGATATTGCTAATATTTTCCGTGCGAAACCTGTGGATGTTTCTGAT |
| | | | | CACACAATAACATTACAACTTGCTGGTGATTTAGACAAAATGGTTGC |
| | | | | GCTACAGAGATTATTGGAGCCTTACGGCATTTGTGAGGTGGCACGG |
| | | | | ACTGGAAGAGTAGCACTAACGAGAGAGTCTAGGGTAGATTCCAAA |
| | | | | TATCTACGAGGATACACTCTTCCGTTGTATGAATGAAAAACTCGGGC |
| | | | | TATGTCTTTCCGACATTACCTTGTCTGATTCCTTCCGCTCTTCTACACT |
| | | | | GCATTGCAAGCAGAACAATTGCCCACAAGTGGAGTAAATTAAAAGG |
| | | | | GGAAACAACAATACCGATCTTTGCTTTGAAGATATACTGATTTGTAT |
| | | | | AACAATAGAGTTTGTATTTGACTAGTATTTATTTGCTCAACGCTGTA |
| | | | | ATAACATATCCCCTTGAGGTTTGATATATGGAAGGAAAAATTACCCAG |
| 1729 | Amaranthus | cDNA | 1254 | GAAGATCTATCTAGAGAGCCTCAAGTGGAACGTTGAGCTTATGCTA |
| | viridis | Contig | | TTAAAGCTTCATTCTAATGCAGATACCCATGCAGAGATAATGTGGTT |
| | | | | AGTGGACATCTTCAGAGCAAAAATTGTCGATATGTCGGAAAGCTTC |
| | | | | GTTACTGTAGAGGTGACTGGTGATCCTGGAAAGATGGCTGCTGTCC |
| | | | | TGAGAAATTTTAGCAAGTATGGAATCAAAGAAGTTGCAAGAACAG |
| | | | | GAAAGATTGCTCTAAGACGAGAAAGGATGGGCCAGACAGCTCCTTT |
| | | | | TTGGAGATTCTCTGCTGCATCTTATCCAGATCTACAAGAAAAGGCTG |
| | | | | TTGAATCTTTTGTCAGGCCTGCCAAAAGAAACATCAACGCTGATCCT |
| | | | | GGCTCATCGTCTAGTGGTGATGTTTATCCAGTGGAGCCTTATGAAG |
| | | | | CCTCCATAAATACAGTACTTGATGCTCACTGGGGCGTTCTTTACGAA |
| | | | | AATGATTCAAGCGGACTTGTATCACATACTCTGTCCATGCTGGTAAA |
| | | | | TAATGCTCCTGGAGTTCTGAACACCGTTACAGGAGTAATTGCTCGTA |
| | | | | GAGGTTATAACATTCAGAGTCTTGCTGTGGGCCCTGCTGAAAAGGA |
| | | | | GGGTCTTTCTCGTATCACAACTGTTGTTCCTGGAAACGACGAGTCAA |
| | | | | TTGCAAAATTGGTTCAGCAATTAAACAAATTAGTAGACCTTCATGAG |
| | | | | ATTCAGGACCTTACGCACCAGCCATTTGCAGAGCGAGAGCTTATGT |
| | | | | TAATTAAAGTAGCGGCAAATACTTCTGCCAGGAGAGACGTCCTTGA |
| | | | | TATTGCTAATATTTTCCGTGCGAAACCTGTGGATGTTTCTGATCACA |
| | | | | CAATAACATTACAACTTGCTGGTGATTTAGACAAAATGGTTGCGCTA |
| | | | | CAGAGATTATTGGAGCCTTACGGCATTTGTGAGGTGGCACGGACTG |
| | | | | GAAGAGTAGCACTAACGAGAGAGTCTAGGGTAGATTCCAAATATCT |
| | | | | ACGAGGATACACTCTTCCGTTGTATGAATGAAAAACTCGGGCTATG |
| | | | | TCTTTCCGACATTACCTTGTCTGATTCCTTCCGCTCTTCTACACTGCAT |
| | | | | TGCAAGCAGAACAATTGCCCACAAGTGGAGTAAATTAAAAGGGGA |
| | | | | AACAACAATACCGATCTTTGCTTTGAAGATATACTGATTTGTATAAC |
| | | | | AATAGAGTTTGTATTTGACTAGTATTTATTTGCTCAACGCTGTAATA |
| | | | | ACATATCCCCTTGAGGTTTGATATATGGAAGGAAAAATTACCCAG |
| 1730 | Amaranthus | cDNA | 1090 | GAAGATCTATCTAGAGAGCCTCAAGTGGAACGTTGAGCTTATGCTA |
| | viridis | Contig | | TTAAAGCTTCATTCTAATGCAGATACCCATGCAGAGATAATGTGGTT |
| | | | | AGTGGACATCTTCAGAGCAAAAATTGTCGATATGTCGGAAAGCTTC |
| | | | | GTTACTGTAGAGGTGACTGGTGATCCTGGAAAGATGGCTGCTGTCC |
| | | | | TGAGAAATTTTAGCAAGTATGGAATCAAAGAAGTTGCAAGAACAG |
| | | | | GAAAGATTGCTCTAAGACGAGAAAGGATGGGCCAGACAGCTCCTTT |
| | | | | TTGGAGATTCTCTGCTGCATCTTATCCAGATCTACAAGAAAAGGCTG |
| | | | | TTGAATCTTTTGTCAGGCCTGCCAAAAGAAACATCAACGCTGATCCT |
| | | | | GGCTCATCGTCTAGTGGTGATGTTTATCCAGTGGAGCCTTATGAAG |
| | | | | CCTCCATAAATACAGTACTTGATGCTCACTGGGGCGTTCTTTACGAA |
| | | | | AATGATTCAAGCGGACTTGTATCACATACTCTGTCCATGCTGGTAAA |
| | | | | TAATGCTCCTGGAGTTCTGAACACCGTTACAGGAGTAATTTCTCGTA |
| | | | | GGGGTTATAACATTCAGAGTCTTGCTGTAGGCCCTGCTGAAAAGGA |
| | | | | GGGTCTTTCTCGTATCACAACTGTCATTCCTGGAAACGACGAGTCGA |
| | | | | TTGGAAAATTGGTTCAGCAATTCAACAAGTTAGTAGACCTTCATGA |
| | | | | GATTCAGGACCTTACTCACCAGCCATTTTCAGAGCGAGAGCTTATGT |
| | | | | TGATCAAAATAGCTGCAAATACTACAGCCAGGAGAGATGTCCTTGA |
| | | | | TATTGCTAATATTTTCCGTGCAAAAGCTGTGGATGTTTCGGATCACA |
| | | | | CGATAACATTACAGCTTGCGGGTGATTTACACAAAATGGTTGCGCT |
| | | | | ACAAAGATTATTGGAGCCTTATGGCATTTGTGAGGTGGCACGGACA |
| | | | | GGAAGAGTCGCACTGAGTAGAGAGTCTGGGGTCGATTCTACATCTT |
| | | | | TACGTGGATACGCTCTTCCTTTGTACGAATAAAAATTCTCCGGCTAT |
| | | | | GTCTTTTCGTCATTATTATGTCCCATTTCCTTCAGCACGTCTGCGCTA |
| | | | | CATTTACATAGCGATCAATTG |
| 1731 | Ambrosia | cDNA | 1616 | CTACCTACTCCGCCATAATCCACACTCAAAGAGCACCTTCTTCTTCTT |
| | trifida | Contig | | CCTAATCTACTTCCATCACCCAAACCTCTAGACCGTACACCGACCAG |
| | | | | TTTCTCTCCAACACTCTCTTCAAATCCTAACAAACCTTATCTCAAATC |
| | | | | ACTCTCTGTGCAAGCAACCAGTGCTTCCGTTTCCACCGCCCTTGATG |
| | | | | CCACTCCATCCATGGCAAGACCTAAGGTGAGGCGCCACACGATTTC |
| | | | | AGTATTTGTTGGTGATGAAAGTGGAATGATAAATCGGATTGCAGGA |
| | | | | GTTTTTGCAAGGAGGGGATATAATATCGAGTCCCTTGCTGTTGGTTT |
| | | | | AAACAAGGATAAGGCTCTTTTTACTATAGTTGTATCGGGAACTGAA |
| | | | | AGAGTGTTGGAGCAAGTTATGAAACAACTTCTAAAGCTCGTTAATG |
| | | | | TTTTAAAGGTTGAAGATATCTCAAAGGAGCCACAAGTAGAACGTGA |
| | | | | GTTGATGCTCATCAAGATAAATGCAGATCCAAGATACCGTTTGGAG |
| | | | | GTCAAGTGGTTAGTGGACATATTTAGAGCTAGAATTGTTGATATTTC |
| | | | | AGAGCACTCTATAACTATTGAGGTAACTGGTGACCCAGGGAAAATG |
| | | | | GTTGCGGTGCAAAGAAATTTAAGCAAGTTTGGGATCCAAGAAGTTG |
| | | | | CTAGGACCGGGAAGATTGCCTTGAGAAGAGAAAGAATGGGTGAAG |
| | | | | ATGCTCCTTTCTGGCGGTTTTCAGCAGCTTCATATCCTGACCTTGAA |
| | | | | GAAATGAACAAAAGAACTCCTCTTCAAGCCAAAAAGAGAACAGAGT |
| | | | | ATCAGGAATCTGATAAGCCTGCTGGGGGAGATGTTTATCCAGTAGA |
| | | | | TTCATCTGATGATTATTCATTCAATCAAGTTCTTGATGCACATTGGG |
| | | | | GTGTTCTTAATGAAGAAGATACAAGCGGGCTTCGTTCCCACACTTTA |
| | | | | TCAATCGTTGTAAATGACTCTCCTGGAGTTCTCAATATCGTAACAGG |
| | | | | AGTTTTTGCTCGCAGAGGCTATAATATTCAGAGTCTAGCAGTCGGTC |
| | | | | ATGCAGAAGTAGAGGGGCGTTCCCGCATCACAACTGTTGTCCCTGG |
| | | | | TACAGATGAGTCAATTGCAAAGTTGGTTCAACAACTATACAAGTTG |
| | | | | ATAGATGTGCATGATGTTGTGGATTTAACGTTGATGCCATTTGCCGA |
| | | | | ACGAGAATTGATGCTGATTAAGATTGCTGTGAATTCCACGGCCCGG |
| | | | | CGCAATGTTCTTGATATTGCAAGCATATTTAGGGCTAAAGCCGTAG |
| | | | | ACGTGTCAGATCATACAATTACACTTGAGGTTACTGGAGATCTGAAT |
| | | | | AAGATGGTTGCACTGCAGAGGTTGTTGGAACCTTGCGGAATTTGTG |
| | | | | AGGTGGCAAGAACAGGTCGCGTGGCACTGACCCGCGAGTCAGGCG |
| | | | | TTGATTCCTCTTACCTTCGTGGATATTCCTATCCTGTGTGATTTCTATC |
| | | | | TTTTATAGCTTCTTCCTGTTTCGGGTAGAGATTGTCAGTAATTTGGG |
| | | | | TGTTCTTTTTCTATCTGGTACCTAGTCTAAGTTGTATGTTTTTAACAA |
| | | | | TAGATTTTTGTTTTTTCATCCGACTTTTAAGACCCTGTTTTGACTCTAT |
| | | | | ATGAAGTTACATACGTAAATACATGCTATG |
| 1732 | Ambrosia | cDNA | 1597 | CCTTTGTCGGGAAAACCATAATGACGAAAACAGCCACCATTTCTTCT |
| | trifida | Contig | | CTCTCCTCGGCGGCTCATTGCCGCCGCCACACCATACAACCGGTGAC |
| | | | | ACTACAACCCACAAACCTAAAACCATTCAATAAAAAATGTTCAGCTC |
| | | | | CTTACACTCTAAAACCACTTAATGCTATCTCTGACAACAAATCTCCG |
| | | | | GCCACATTGCCACATATTCTTCCCCTTAACACACCACTTACTTCCAAG |
| | | | | GTGAAGCGTCACACGATCTCGGTGTTTGTTGGGGATGAAAGTGGTA |
| | | | | TTATAAATCGAATTGCGGGGGTGTTTGCTAGACGAGGTTATAATAT |
| | | | | CGAGTCGCTTGCGGTTGGTTTGAATAAGGATAAGGCTTTGTTTACT |
| | | | | ATTGTTGTCTCTGGGACTGAGAAGGTGTTGCAGCAAGTTGTAGAGC |
| | | | | AACTTAACAAGCTTGTTAATGTCTTGAAGGTGGAAGATCTTTCTCGG |
| | | | | GATCCGCAAGTAGAGCGGGAATTGATGCTTGTGAAGCTTAATGTTG |
| | | | | ATCCAAGCACAAAAGCTGAGATTATGTGGTTAGTAGACATCTTTAG |
| | | | | AGGCAATGTGGTGGATGCATCGGAATCCTCATTGACTATAGAGGTT |
| | | | | ACTGGGGATCCCGGGAAGATAGCTGCTGTTCAGAGGAATCTAGCTA |
| | | | | AGTTTGGAATCAAAGAACTTACAAGAACAGGAAAGATTGCGCTAAG |
| | | | | ACGGGAAAAAATGGGTGAAACTGCTCCGTTTTGGAACTTCTCTGCA |
| | | | | GCATCTTATCCAGATCTAGAAGCCCTGGGTCCTGTTGCTAGTATTAC |
| | | | | TAAAGTGGTTGACGAGACTCCTAGTACAACGTCAGGGGGGGGCGA |
| | | | | TGTTTATCCTGTGGATTCCTACGATAGCTGCTACGTAAGGGATCGAG |
| | | | | TTCTCGATGCCAATTGGGGCGTGCTATATGACGAAGATGCAACGGG |
| | | | | TCGTCAATCTCACACTTTGAACATTCTTGTGAATAATGCTCCTGGAG |
| | | | | TTCTCAACCTGGTCACCGGAGTTATATCCAGACGGGGTTATAACGTT |
| | | | | CAGAGTCTGGCTGTAGGTCCTGCAGAACTGGAAGGTTTATCTCGCA |
| | | | | TTACTACTGTGATTGCCGGCACAGACGAGTCTATTGGCAAGTTGGTT |
| | | | | CAGCAGTTCTACAAATTGATAGATGTTCATGAGGTCAAAGATATCA |
| | | | | CCTACTTACCATTTTCGGAACGCGAGTTAATGCTGATCAAAGTTGCT |
| | | | | GCAAGCCCTGCTGCTAGAAGGGATGTATTGGACATTGCCACCATCT |
| | | | | TCCGCGCCAAGCCTGTTGATGTTTCTGACCATACTATTACGCTAGAA |
| | | | | CTCACCGGAGACTTCAATAAGCTATTCGCCCTGCAAACGTTATTGGA |
| | | | | GACCTATGGAATTTGCGAGGTCGCAAGGACTGGTCGAGTGGCATT |
| | | | | GGTTAGAGAGTCAGGAGTCGATTCTACATATCTACGTGGGTTCTCT |
| | | | | ATGCCTCCAACTTGATTCAAGCCGATCCGATGGTTGCAAGGGTCCCC |
| | | | | ATCCGGTGGGCTACTTTTATGTTGCGACATGTAACTGTTACAGTTAT |
| | | | | TAGTCTTTATTGTTTGTTAGCTTAATATGTTGAAGACAGTTTCTAGTA |
| | | | | ATCATATGGTTTCACA |
| 1733 | Ambrosia | gDNA | 3632 | GTCATGTATTGCATTCTATGAGACTATGACTACTCAGGGACTATTTT |
| | trifida | Contig | | ATCAGTTTCTACAAGCTTACAGTGCTTATTGTCATGCTTTATGGAAG |
| | | | | TCTATGAAGTTTATTTTCGTTACAGTGCTTATGGTCTAATTTTGTTTT |
| | | | | GACTCAAGAAAACCTGTATATTTGTTTGAGTGATTTTTAATAAGACA |
| | | | | AAAATCTGTGGTTGCTATCATTATGCAATTTCTGAGTAAATTTGTTTT |
| | | | | TCTAGTTCCAAAACACATAATCGTCTTAGGCTCGGGACTTCACAATT |
| | | | | TTCTTTTTAATCTAACATACATAATCGTGTTTACGACTCATAATCAAT |
| | | | | ATGATTTGCTCTAATGTACCGGTAAAATAAACACTTTGTATCGCTTT |
| | | | | GTGCTATCATAAAGAGCTGATTGCCTGATAGAAGTTCTAAATAAATT |
| | | | | CTTTTTTTTTACGTTGGAATCTAGGTAACTGGTGACCCAGGGAAAAT |
| | | | | GGTTGCGGTGCAAAGAAATTTAAGCAAGTTTGGGATCCAAGAAGTT |
| | | | | GCTAGGACCGGGAAGGTAAGTAAATAGTGCATTCATAGTGTATTCA |
| | | | | AATGCATTAAACCTCCTAAGTTTATCTATTCTTTCTTTTGAAATAGTA |
| | | | | TTGTAATGATATAATTATTTTAATCATATTTAGTACTTAAGTTATTTG |
| | | | | CAAAATTCAAAAAGTGAATGAAAAGTTATTGGGGTCTGGCGGTCTG |
| | | | | CTCAACGTGAGCTGACTTGGACTTATAGTTACCTCCCTGACCATCTTT |
| | | | | AATGCTTTAGATTGCCTTGAGAAGAGAAAGAATGGGTGAAGATGCT |
| | | | | CCTTTCTGGCGGTTTTCAGCAGCTTCATATCCTGACCTTGAAGAAAT |
| | | | | GAACAAAAGAACTCCTCTTCAAGCCAAAAAGAGAACAGAGTATCAG |
| | | | | GAATCTGATAAGCCTGCTGGGGTAAGTTTCATATATTTTTGTTTTTG |
| | | | | CTGAAAGTTGTATTACCTTTTGATTAATTATAAACATTTTTTGTTTGT |
| | | | | GAAGGGAGATGTTTATCCAGTAGATTCATCTGATGATTATTCATTCA |
| | | | | ATCAAGTTCTTGATGCACATTGGGGTGTTCTTAATGAAGAAGATGT |
| | | | | AAGACCTTATTGTCAATTCTTAATTTCCACAAACATCATAAAAACAT |
| | | | | GGGAAGCATTTCAACAATTTTTTGTTTAATGTTTTTAGACAAGCGGG |
| | | | | CTTCGTTCCCACACTTTATCAATCGTTGTAAATGACTCTCCTGGAGTT |
| | | | | CTCAATATCGTAACAGGAGTTTTTGCTCGCAGAGGCTATAATATTCA |
| | | | | GGTATACTTTTTGTGGTGTTGAATGTTGATTTTACGTTTGTTTTCTTA |
| | | | | TTGTAATTATTTTAACAATTGCCAGAAATTGAAGTCTTTTTGGTGTTT |
| | | | | GTATTTTTTAAATATCAGAGTCTAGCAGTCGGTCATGCAGAAGTAG |
| | | | | AGGGGCGTTCCCGCATCACAACTGTTGTCCCTGGTACAGATGAGTC |
| | | | | AATTGCAAAGTTGGTTCAACAACTATACAAGTTGATAGATGTGCAT |
| | | | | GATGTGAGTATATATCACCGTTGGAAATTGCTTCATTTACTCGATTC |
| | | | | ATGTTATGTTTTAACTCAGATGAGTAAAGCTAAATGAAATTAACTAA |
| | | | | AAAGGAAAAGGATCGTAAGAGTCTAAAGTCTCTTGAAGTGTATTTT |
| | | | | TAATTAATAAAACCTCTAAAATCATTTTATTAATTCCATGATCATATC |
| | | | | TGACACTTTTATTTTTTGTGACAATCCAGTCTGCGCAAACAATTAGCT |
| | | | | GCATTTTGCTACTTGTACCCATTATGTTTAGTTTTTTGAGATGGTTCA |
| | | | | TAGGTCATTTCTCAGATTTTGAGGTTGCTCCGCTGAATTCATTCAATT |
| | | | | GAGTGACCAACAAACATATTTTTGTTTGTTCAGGTTGTGGATTTAAC |
| | | | | ATTGATGCCATTTGCCGAACGAGAATTGATGCTGATTAAGATTGCT |
| | | | | GTGAATTCAACGGCCCGACGCAATGTTCTTGATATTGCAAGCATATT |
| | | | | TAGGGCTAAAGCCGTAGATGTGTCAGATCATACAATTACACTTGAG |
| | | | | GTGAATTTTATTCACTTGCAGTTTATTTTCTGTCAAGTGAAAATTCTA |
| | | | | ATACTTATATTTTGAGTCGTTTCTAGAAAATTGCTCCATTTCTTCTTTT |
| | | | | GGGGAAAATTCCAGCTAGACCAAGGACAAGTGATGTGCTAATCTAA |
| | | | | TCCCATGCTTGTTCACAAAATAGAAACCACTAGTGAGACTAACCAG |
| | | | | AAGCCATCTTCATCTAAAGCCCATGCTTGTTGTTTGAGTGTTTGATG |
| | | | | TGAACCCAACCTTAGATAAATATGGTTCTGGGCCAAGTCTCATTACT |
| | | | | AGTGTTTAACTTACGATCATGGGGTTTTACATGATGTTAGGGAGGA |
| | | | | TAGTTAAGATTAGTGGTTGTCGGCCAAGCAAAATCATGTGGGGTTA |
| | | | | TTTATTTTATAAAAGAAATTTAGCCCAATTAAAAATCAAAAAAATAA |
| | | | | AAATAAGAAATACATACATTCGATTATGAACTATTTTAGAAGTATGA |
| | | | | TGCTCTTTTTTCTTCCAAAAGTAACCAAGATAGAACATAGTTTCGAA |
| | | | | GTTCAGTGGCATGGTGTGTGGAAAGTCTACATTGGAATGCATTTTG |
| | | | | TTATTGAATATAGTATTTAAATTATATATTTTTGAATAAATATTTAAG |
| | | | | CTTTTCAAATCGAGCTTGAGCCGATCTCAAATCCTCCTTGAGCTGAT |
| | | | | CTTAAATCATCCTTGAGCCGAGGTCATGCATGGCTACATTACCTTTT |
| | | | | GTTTGGTTTCTATACAAGAACCAATGCAACACAAACCGCATAGCATA |
| | | | | TACAATATGGCACATTATAAAGCTTCACTGAAACATTAAATCAATCA |
| | | | | CTGCATGTTCTTTATGATCCCTTATTTGTTTTACGTGTATTTTCTAGGT |
| | | | | TACTGGAGATCTGAATAAGATGGTTGCACTGCAGAGGTTGTTGGAA |
| | | | | CCTTGCGGAATTTGTGAGGTCTGTTTGTGAATCCCTTGTCTCCAATC |
| | | | | ATATTTTTATTAAACCCGACTTCATTCAAGGTTTAGACTTACACCCCT |
| | | | | GCTCGACGATCATTTGCAGGTGGCAAGAACAGGTCGCGTGGCACT |
| | | | | GACCCGCGAGTCAGGCGTTGATTCCTCTTACCTTCGTGGATATTCCT |
| | | | | ATCCTGTTTGATTTCTATCTTTTATAGCTTCTTCCTGTTTCGGGTAGA |
| | | | | GATTGTCAGTAATTTGGGTGTTCTTTTTCTATCTGGTACCTAGTCTAA |
| | | | | GTTGTATGTTTTTAACAATAGATTTTTGTTTTTTCATCCGACTTTTAA |
| | | | | GACCCTGTTTTGACTCTATATGAAGTTACATACGTAAATACATGCTA |
| | | | | TGTGTGGCCCTTTCTAGAAAAAGGTGAGTTAGGCATCGTGGGTTCG |
| | | | | GGTTAAACATGTCATTTATTGGTACAAGTCGCGGCGACTAGCTGGT |
| | | | | GGGCTGGGTTGCGGCAACCCACAAAACACTTGTTTGTTTGATTTTAG |
| | | | | TTTACCTTTTTTGAACAGGGAATTTATTTTACTTCTTAGAAGGTTTGT |
| | | | | GTTGATAGAATAGTTAATTCGATGACTAGTATGGTTTTGAAAACATT |
| | | | | GCATAAACCTTATTGAGATTGGCAGGAGGTAGGAATTAACTTGTAG |
| | | | | GAATTGATAGCCATTATGTGAAGTGAGGGGATGGTAAAGCAAAGC |
| | | | | TGATTATTGGGTG |
| 1734 | Ambrosia | gDNA | 1966 | GGTTACTGGGGATCCCGGGAAGATAGCTGCTGTTCAGAGGAATCTA |
| | trifida | Contig | | GCTAAGTTTGGAATCAAAGAACTTACAAGAACAGGAAAGGTAATTG |
| | | | | TTTAACGACTGTTTGTTTACGATGATTGGTTCTTGACTGAATTTGCTG |
| | | | | CTGCTTGTATGTAGATTGCGCTAAGACGGGAAAAAATGGGTGAAAC |
| | | | | TGCTCCGTTTTGGAACTTCTCTGCAGCATCTTATCCAGATCTAGAAG |
| | | | | CCCTGGGTCCTGTTGCTAGTATTACTAAAGTGGTTGACGAGACTCCT |
| | | | | AGTACAACGTCAGGGGTAGGTTGTTAAATTTTCAGGCTTCCTTCGCA |
| | | | | GCAATCATTGTTCTCTAACTCGGTATATTTTTATTTGAATGAAGGGG |
| | | | | GGCGATGTTTATCCTGTGGATTCCTACGATAGCTGCTACATAAGGG |
| | | | | ATCGAGTTCTCGATGCCAATTGGGGCGTGCTATATGACGAAGACGT |
| | | | | AAGTCATCGTCTCGTTACTATGTTTACGTTTCATTTCTGACACGCAAA |
| | | | | TTAATGCTGCAGGCAACGGGTCGTCAATCTCACACTTTGAACATTCT |
| | | | | TGTGAATAATGCTCCTGGAGTTCTCAACCTGGTCACCGGAGTTATAT |
| | | | | CCAGACGGGGTTATAACGTTCAGGTAAATTAACTTGTTTTAAGCAAT |
| | | | | CCGGTCCAGGCCTAGCTAACTTTAACGGTAATTGGTTGTTTGTTTGT |
| | | | | TTACAGAGTCTGGCTGTAGGTCCTGCAGAACTGGAAGGTTTATCTC |
| | | | | GCATTACTACTGTGATTGCCGGCACAGACGAGTCTATTGGCAAGTT |
| | | | | GGTTCAGCAGTTCTACAAATTGATAGATGTTCATGAGGTACAAAAG |
| | | | | TGTCCGTTATCGTTTTCACTGGTATGGAAAAATTAATGAAATACTAC |
| | | | | TCAGGTCAAAGATATCACCTACTTACCATTTTCGGAACGCGAGTTAA |
| | | | | TGCTGATCAAAGTTGCTGCAAGCCCTGCTGCTAGAAGGGATGTATT |
| | | | | GGACATTGCCACCATCTTCCGCGCCAAGCCTGTTGATGTTTCTGACC |
| | | | | ATACTATTACGCTAGAAGTAATGTTTCTTAATCCTTGTTTCTTTTTGTT |
| | | | | TTTCCTGTTTCTGATATCATCTTCCGTCTTCTATGTTATGTTCTAGCTC |
| | | | | ACCGGAGACTTCAATAAGCTATTCGCCCTGCAAACGTTATTGGAGA |
| | | | | CCTATGGAATTTGCGAGGTTTGTAACAGCATAATTAATTGTTTGTTT |
| | | | | GTTGGTAACAGTGCATGGTTGGTTACGTTATATTGTGTGTGTAGGT |
| | | | | CGCAAGGACTGGTCGAGTGGCATTGGTTAGAGAGTCAGGAGTCGA |
| | | | | TTCTACATATCTACGTGGGTTCTCTATGCCTCCAACTTGATTCAAGCC |
| | | | | GATCCGATGGTTGCAAGGGTCCCCATCCGGTGGGCTACTTTTATGTT |
| | | | | GCGACATGTAACTGTTACAGTTATTAGTTTTTATTGTTTGTTAGCTTA |
| | | | | ATATGTTGAAGACAGTTTCTAGTAATCATATTGGTTTCACATCAGAT |
| | | | | CAGATGCTATATATGCTAATAACTTGCAAAGTTCAGATCGCCTGCAG |
| | | | | AAGGTAAGCGATGAGTATTTAGACCAGACCAACTTATTATACGATA |
| | | | | TCAACATATTAATTAATATTGCCCTATCGGCAATACATTGCATTTTCA |
| | | | | CTTATCGAATTCTCAGGTTTCTGGATTTCTGCCCCATAACACACATGA |
| | | | | ACTCTGGACAAAAAATAATAATAATAATAATAATAATTTTAACAAGT |
| | | | | TATTGCCGATTTTTCTAGATGCTACGTTAAATCTTCAATAAAAAAACT |
| | | | | AAAGGAAAAAAAAAAATCAAAATTGCAGCTATTGAGTTATCTACCC |
| | | | | CAGATCCCTGCCACCGTGGACCACGCATGGGTGATGCGATGTTCCA |
| | | | | CGAATGAATCATTTTATGCCCTTAACACCACATGATCTTAGACTAGA |
| | | | | CGGTGTGGTTATAGGGTGAAGGGGGCATGAAGCGACATGTGACA |
| 1735 | Ambrosia | gDNA | 1865 | GGGTCCTGTTGCTAGTATTACTAAAGTGGTTGACGAGACTCCTAGT |
| | trifida | Contig | | ACAACGTCAGGGGTAGGTTGTTAAATTTTCAGGCTTCCTTCGCAGCA |
| | | | | ATCATTGTTCTCTAACTCGGTATATTTTTATTTGAATGAAGGGGGGC |
| | | | | GATGTTTATCCTGTGGATTCCTACGATAGCTGCTACGTAAGGGATCG |
| | | | | AGTTCTCGATGCCAATTGGGGCGTGCTATATGACGAAGATGTAAGT |
| | | | | CATCGTCTCGTTACTATGTTTACGTTTCATTTCTGACACGCAAATTAA |
| | | | | TGCTGCAGGCAACGGGTCGTCAATCTCACACTTTGAACATTCTTGTG |
| | | | | AATAATGCTCCTGGAGTTCTCAACCTGGTCACCGGAGTTATATCCAG |
| | | | | ACGGGGTTATAACGTTCAGGTAAATTAACTTGTTTTAAGCAATCCGG |
| | | | | TCCAGGCCTAGCTAACTTTAACGGTAATTGGTTGTTTGTTTGTTTGTT |
| | | | | TACAGAGTCTGGCTGTAGGTCCTGCAGAACTGGAAGGTTTATCTCG |
| | | | | CATTACTACTGTGATTGCCGGCACAGACGAGTCTATTGGCAAGTTG |
| | | | | GTTCAGCAGTTCTACAAATTGATAGATGTTCATGAGGTACAAAAGT |
| | | | | GTCCGTTATCGTTTTCACTGGTATGGAAAAATTAATGAAATACTACT |
| | | | | CAGGTCAAAGATATCACCTACTTGCCATTTTCGGAACGCGAGTTAAT |
| | | | | GCTGATCAAAGTTGCTGCAAGCCCTGCTGCTAGAAGGGATGTATTG |
| | | | | GACATTGCCACCATCTTCCGCGCCAAGCCTGTTGATGTTTCTGACCA |
| | | | | TACTATTACGCTAGAAGTAATGTTTCTTAATCCTTGTTTCTTTTTGTTT |
| | | | | TTCCTGTTTCTGATATCATCTTCCGTCTTCTATGTTATGTTCTAGCTCA |
| | | | | CCGGAGACTTCAATAAGCTATTCGCCCTGCAAACGTTATTGGAGAC |
| | | | | CTATGGAATTTGCGAGGTTTGTAACAGCATAATTAATTGTTTGTTTG |
| | | | | TTGGTTGGTCACAGTGCATGGTTGGTTACGTTATATTGTGTGTGTAG |
| | | | | GTCGCAAGGACTGGTCGAGTGGCATTGGTTAGAGAGTCAGGAGTC |
| | | | | GATTCTACATATCTACGTGGGTTCTCTATGCCCTTGTGATAGACGCA |
| | | | | CAAGTCCAACTTGATTCAAGCCGATCCGATGGTTGCAAGGGTCCCC |
| | | | | ATCCGGTGGGCTACTTTTATGTTGCGACATGTAACTGTTACAGTTAT |
| | | | | TAGTTTTTATTGTTTGTTAGCTTAATATGTTGAAGACAGTTTCTAGTA |
| | | | | ATCATATTGGTTTCACATCAGATCAGATGCTATATATGCTAATAACTT |
| | | | | GCAAAGTTCAGATCGCCTGCAGAAGGTAAGCGATGAGTATTTAGAC |
| | | | | CAGACCAACTTATTATACGATATCAACATATTAATTAATATTGCCCTA |
| | | | | TCGGCAATACATTGCATTTTCACTTATCGAATTCTCAGGTTTCTGGAT |
| | | | | TTCTGCCCCATAACACACATGAACTCTGGACAAAAAATAATAATAAT |
| | | | | AATAATAATTTTAACAAGTTATTGCCGATTTTTCTAGATGCTACGTTA |
| | | | | AATCTTCAATAAAAAAACTAAAGGAAAAAAAAATCAAAATTGCAGC |
| | | | | TATTGAGTTATCTACCCCAGATCCCTGCCACCGTGGACCACGCATGG |
| | | | | GTGATGCGATGTTCCACGAATGAATCATTTCATGCCCTTCACACCAC |
| | | | | ATGATCTTAGACTAGACGGTGTGGTTATAGGGTGAAGGGGCATAG |
| | | | | AGCGACATGTGCCAAATCTCTACCCAACCTGTCCAACGCCACAGAT |
| | | | | GGGGATGAATGCAATACGAAGAGAGGTAGAAAGCCAAGCACTGCA |
| | | | | CGGGGCCTCTCTGATGCCTACACTTTCCCGGGGGAGAG |
| 1736 | Ambrosia | gDNA | 1079 | TCTTTAACAAACTGTGTATGTTAAAAGGACTATAATGCCCCAGTTAT |
| | trifida | Contig | | ATGTTGATAACTAAGTGTGTGGTATGATTAATGAGCAGGGTGAAGC |
| | | | | GTCACACGATCTCGGTGTTTGTTGGGGATGAAAGTGGTATTATAAA |
| | | | | TCGAATTGCGGGGGTGTTTGCTAGACGAGGTTATAATATCGAGTCG |
| | | | | CTTGCGGTTGGTTTGAATAAGGATAAGGCTTTGTTTACTATTGTTGT |
| | | | | CTCTGGGACTGAGAAGGTGTTGCAGCAAGTTGTAGAGCAACTTAAC |
| | | | | AAGCTTGTTAATGTCTTGAAGGTCTCATCTTGTAATTTACGCTGCTAT |
| | | | | CGTCGTTCATTAATAACGTTGTTCCTAATGCGTTACTCAGTTTTGTTT |
| | | | | GATGTACCAGGTGGAAGATCTTTCTCGGGATCCGCAAGTAGAGCG |
| | | | | GGAATTGATGCTTGTGAAGCTTAATGTTGATCCAAGCACAAAAGCT |
| | | | | GAGGTACGTACTTATATGATGTTTCTTGCAATTTATCATTTAATTATG |
| | | | | CGTTCCTGTTCAATGGTCGGGTCAACAAACAAGTGTCGCGTGTTGTT |
| | | | | TAAAAATGTAGTTTGTTTAAGTTGATAAATATTACTTACTTTTACCTT |
| | | | | CAGATTATGTGGTTAGTAGACATCTTTAGAGGCAATGTGGTGGATG |
| | | | | CATCGGAATCCTCATTGACTATAGAGGTGACTTCCTCCTATTCATCA |
| | | | | AGCTTGTGTGTTTATACAAATTATTAAGCGATTTTGATGTTTATGTC |
| | | | | GTCAGTAATAGTTCTCATATGGTTAATCTAGGTTACTGGGGATCCCG |
| | | | | GGAAGATAGCTGCTGTTCAGAGGAATCTAGCTAAGTTTGGAATCAA |
| | | | | AGAACTTACAAGAACAGGAAAGGTAATTGTTTAACGACTGTTTGTT |
| | | | | TACGATGATTGGTTCTTGACTGAATTTGCTGCTGCTTGTATGTAGAT |
| | | | | TGCGCTAAGACGGGAAAAAATGGGTGAAACTGCTCCGTTTTGGAAC |
| | | | | TTCTCTGCAGCATCTTATCCAGATCTAGAAGCCCTGGGTCCTGTTGC |
| | | | | TGCCGTTTCTAAAGTGGCTGACGAGACTCCTAGTCCAACGTCAGGG |
| | | | | GTAGGT |
| 1737 | Ambrosia | gDNA | 990 | AAAATTTCTATGTTTTATGTTGTGATGAATATATGGTCTATAATGTAT |
| | trifida | Contig | | TTATGCTTTATGTTGTGGTGAATATAATCTTGATGTCATGCTTTTGTT |
| | | | | ATTCAGGGTGAGGCGCCACACGATTTCAGTATTTGTTGGTGATGAA |
| | | | | AGTGGAATGATAAATCGGATTGCAGGAGTTTTTGCAAGGAGGGGA |
| | | | | TATAATATCGAGTCCCTTGCTGTTGGTTTAAACAAGGATAAGGCTCT |
| | | | | TTTTACTATAGTTGTATCGGGAACTGAAAGAGTGTTGGAGCAAGTT |
| | | | | ATGAAACAACTTCTAAAGCTCGTTAATGTTTTAAAGGTTGGCTTTTA |
| | | | | TTCTGATGGTTGATTCATCATATTGTCTATTTTTTATACTTTACTTATA |
| | | | | TCAACCATTTTTAATAGGTTGAAGATATCTCAAAGGAGCCACAAGTA |
| | | | | GAACGTGAGTTGATGCTCATCAAGATAAATGCAGATCCAAGATACC |
| | | | | GTTTGGAGGTGTTTAACTAATTATTTTATGATCTTCATGTTTTGGTTA |
| | | | | ATTACGGTTATTTATATTATACCTAAGTACTGCAATGAACCGTTCTTA |
| | | | | TAATTGCAATGAACACATGCAATCAAATATTCAAATATAGCTAACCA |
| | | | | TTTTTAGCCTAAGGATATGCTATCTAACCATGTTCTTTTGTGATTCTA |
| | | | | ATTTTCAGGTCAAGTGGTTAGTGGACATATTTAGAGCTAGAATTGTT |
| | | | | GATATTTCAGAGCACTCTATAACTATTGAGGTGACGGGAGTCGAAC |
| | | | | TTTTTCTATAGCTTTATTTATAATTCTTCACGCATCATCTAGGAGTGA |
| | | | | GTTTGGATCTTAATGGATTGAATTGTTTATGCTATTATAGGGCCTTT |
| | | | | GCTAATTTGCATATGTTCACAACCTTATTTATTAGAATTTAGATATAA |
| | | | | GGGTTGTATACTCTGTGACTACTTGGAACAATTACAACACTAAGTAA |
| | | | | TACAACATATGCTAGGAAGGTGTTAGACCGTTACAATCGACACCCAA |
| 1738 | Ambrosia | gDNA | 984 | GGGTTTGGTGTTTATTTTTTTTAAATATCAGAGTCTAGCAGTCGGTC |
| | trifida | Contig | | ATGCAGAAGTAGAGGGGCGTTCCCGCATCACAACTGTTGTCCCTGG |
| | | | | TACAGATGAGTCAATTGCAAAGTTGGTTCAACAACTATACAAGTTG |
| | | | | ATAGATGTGCATGATGTGAGTATATATCACCGTTGGAAATTGCTTCA |
| | | | | TTTACTCGATTCACGTTATGTTTTAACTCAGATGAGTAAAGCTAAAT |
| | | | | GAAATTAACTAAAAAGGAAAAGGATCGTAAGAGTCTAAAGTCTCTT |
| | | | | GAAGTGTATTTTTAATTAATAAAACCTCTAAAATCATTTTATTAATTC |
| | | | | CATGATCATATCTGACACTTTTATTTTTGTGACAATCCAGTCTGCGCA |
| | | | | AACAATTAGCTGCATTTTGCTACTTGTACCCATTATGTTTAGTTTTTT |
| | | | | GAGATGGTTCATAGGTCATTTCTCAGATTTTGAGGTTGCTCCGCTGA |
| | | | | ATTTTAGCTAAACAAACGAAAACAAACATATTTTTGTTTGTTCAGGT |
| | | | | TGTGGATTTAACATTGATGCCATTTGCCGAACGAGAATTGATGCTG |
| | | | | ATTAAGATTGCTGTGAATTCCACTGCCCGACGCAATGTTCTTGATAT |
| | | | | TGCAAGCATATTTAGGGCTAAAGCCGTAGATGTGTCAGATCATACA |
| | | | | ATTACACTTGAGGTGAATTTTATTCACTTGCAGTTTATTTTCTGTCAA |
| | | | | GTGAAAATTCTAATGCTTATATTTTGAGTCGTTTCTAGAAAATTGCTC |
| | | | | CATTTCTTCTTTTGGGGAAAATTCCAGCTAGACCAAGGACAAGTGAT |
| | | | | GTGCTAATCTAATCCCATGCTTGTTCACAAAATAGAAACCACTAGTG |
| | | | | AGACTAACCAGAAGCCATCTTCATCTAAAGCCCATGCTTGTTGTTTG |
| | | | | AGTGTTTGATGTGAACCCAACCTTAGATAAATATGGTTCTGGGCCA |
| | | | | AGTCTCATTACTAGTGTTTAACTTACGATCATGGGGTTTTACATG |
| 1739 | Ambrosia | gDNA | 804 | TTAGGTGGAATAACAAATGTGTATAGAGGTTGAGAAAACAGAATA |
| | trifida | Contig | | ATGTAGATATCTATATCTTTAACAAACTGTGTATGTTAAAAGGACTA |
| | | | | TAATGCCCCAGTTATATGTTGATAACTAAGTGTGTGGTATGATTAAT |
| | | | | GAGCAGGGTGAAGCGTCACACGATCTCGGTGTTTGTTGGGGATGA |
| | | | | AAGTGGTATTATAAATCGAATTGCGGGGGTGTTTGCTAGACGAGGT |
| | | | | TATAATATCGAGTCGCTTGCGGTTGGTTTGAATAAGGATAAGGCTTT |
| | | | | GTTTACTATTGTTGTCTCTGGGACTGAGAAGGTGTTGCAGCAAGTT |
| | | | | GTAGAGCAACTTAACAAGCTTGTTAATGTCTTGAAGGTCTCATCTTG |
| | | | | TAATTTACGCTGCTATCGTCGTTCATTAATAACGTTGTTCCTAATGCG |
| | | | | TTACTCAGTTTTGTTTGATGTACCAGGTGGAAGATCTTTCTCGGGAT |
| | | | | CCGCAAGTAGAGCGGGAATTGATGCTTGTGAAGCTTAATGTTGATC |
| | | | | CAAGCACAAAAGCTGAGGTACGTACTTATATGATGTTTCTTGCAATT |
| | | | | TATCATTTAATTATGCGTTCCTGTTCAATGGTCGGGTCAACAAACAA |
| | | | | GTGTCGCGTGTTGTTTAAAAATGTAGTTTGTTTAAGTTGATAAATAT |
| | | | | TACTTACTTTTACCTTCAGATTATGTGGTTAGTAGACATCTTTAGAGG |
| | | | | CAATGTGGTGGATGCATCGGAATCCTCATTGACTATAGAGGTGACT |
| | | | | TCCTCCTATTCATCAAGCTTGTGTGTTTATACAAATTAAGCGATTTTG |
| | | | | ATGTTTATGT |
| 1740 | Ambrosia | gDNA | 409 | AAATTAATTTTGGGTTTTTTACACTTCTAGTTTTATTCTTTATAAAATT |
| | trifida | Contig | | TCTATGTTTTATGTTGTGATGAATATATGGTCTATAATGTATTTATGC |
| | | | | TTTATGTTGTGGTGAATTTAATCTTGATGTCATGCTTTTGTTATTCAG |
| | | | | GGTGAGGCGCCACACGATTTCCGTATTTGTTGGTGATGAAAGTGGA |
| | | | | ATGATAAATCGGATTGCAGGAGTTTTTGCAAGGAGGGGATATAATA |
| | | | | TCGAGTCCCTTGCTGTTGGTTTAAACAAGGATAAGGCTCTTTTTACT |
| | | | | ATAGTTGTATCGGGAACTGAAAGAGTGTTGGAGCAAGTTATGAAAC |
| | | | | AACTTCTAAAGCTCGTTAATGTTTTAAAGGTTGGCTTTTATTCTGATG |
| | | | | GTTGATTCATCATATTGTCTATTTTTTATAC |
| 1741 | Conyza | cDNA | 1705 | TGGCCATTACGGCCGGGGCTGCAGATTGGAACTACCACCTAAACTG |
| | canadensis | Contig | | CTTTCTTTCACTAAATCAAGTGTTGGTCGAAGGCTTGCTTTTAAGCT |
| | | | | GGTTAATGCTATCTCCGACTCCGGTAATGGAGCTCCGGCTTCACTTC |
| | | | | TTTCTACTTCTTCCCTTAATGGAGTTACTGCCCCGCCACTTTCTTCCA |
| | | | | AGGTGAAGCGCCACACTATCTCGGTGTTCGTAGGAGATGAAAGTG |
| | | | | GTATTATAAACCGAATCGCAGGAGTTTTCGCTAGAAGAGGTTATAA |
| | | | | TATCGAGTCACTTGCTGTTGGTTTAAACAAGGACAAGGCTTTGTTTA |
| | | | | CTATAGTTGTTTCTGGAACTGAAAAGGTCTTGCAACAAGTGGTGGA |
| | | | | GCAGCTCAACAAGCTTGTGAACGTCATAAAGGTGGAAGACCTTTCC |
| | | | | AAGGAACCACAGGTAGAACGGGAATTGATGCTTGTGAAGCTTAAT |
| | | | | GTAGATCCAACCACACGAGCTGAGATTATGTGGCTAGTAGACGTCT |
| | | | | TTAGAGCCAACGTTGTGGATGCATCAGAAAGCTCGTTGACTATAGA |
| | | | | GGTTACCGGGGATCCTGGGAAGATTGTTGCTGTTCAGAGAAATTTA |
| | | | | GCCAAGTTCGGAATCCAAGAGCTCACTAGAACAGGAAAGATCGCTC |
| | | | | TAAGACGGGAAAAACTGGGTGAAACTGCTCCATTTTGGAACTTCTC |
| | | | | TGCAGCATCTTACCCAGATCTAGAATCCGCAACTCCAATTGCTACTT |
| | | | | CTTCAAGTGTTACTTGCACTGTTGATGAGGATTCGACTGCAATGTCA |
| | | | | GGGGGTGATGTGTATCCTGTGGAGTACAACAATAGCTGCATAATGG |
| | | | | ATCAAGTTCTTGACGCCAATTGGGGGGTAGTCTATGATGAAGATTC |
| | | | | AACTGGTCGGCAGTCTCATACTTTAAACATTCTGGTCAATGATACTC |
| | | | | CTGGAGTTCTCAACGTGGTCAGTGGAGTTATATCCAGAAGGGGTTA |
| | | | | CAACATTCAGAGTCTGGCCGTAGGTCCAGCAGAAATGGAGGGTTTA |
| | | | | TCTCGCATTACGACTGTGATTGCTGGTACAGATGAATCTATTTACAA |
| | | | | GTTGGTTCAGCAGTTCCGCAAATTGGTGGATGTTCATGAGGTCAAA |
| | | | | GATATCACCCATCTACCATTTGCAGAACGTGAGCTAATGTTAATTAA |
| | | | | AGTTGCTGCTAGCTCTGCTGTTCGAAGGGATGTCCTAGACATCGCC |
| | | | | ACCATCTTTCGAGCCAAGCCTGTTGATGTTTCTGATCACACTCTCACA |
| | | | | CTAGAACTCACAGGAGACTTCAACAAGTTGTTTGCATTGCAAAGATT |
| | | | | GTTAGAGTCCTATGGAATTTGTGAGGTCGCAAGGACTGGTCGAGTG |
| | | | | GCATTGGTGCGAGAGTCAGGGGTCGATTCTACATATCTACGTGGGT |
| | | | | ACTCCCTTCCGTTATAATAACACCGGAAGTTATCCCTCAATTGTATGT |
| | | | | TGCATTATTGTAGCTGCAAGTGAACACAACTGTTCAGTGTCTTTTGC |
| | | | | ACTGATGGCTGCGGATGTGAGAGTATGACTTCTTGCTAGAAAAAGA |
| | | | | AAAAAGGAATGTTTAGTAGATCAATTTATGTTGTATTATGGTATGCC |
| | | | | GTGTGATGTCAAAACTGTAACAGCTTTCATTCATAGAAGTTTCATAC |
| | | | | TTAAAATGTGATGTTAAGCAAATTTATAAGTAAAAAAAAAAGAAAA |
| | | | | AAAAACAAAACATGTCGGCCGCCTCGGTCTCTA |
| 1742 | Conyza | cDNA | 498 | TTTGGGATTCGAGAAGTTGCAAGGACTGGAAAAATTGCTTTGAGAA |
| | canadensis | Contig | | GAGAAAAAATGGGCGAGGATGCTCCTTTCTGGCGTTTTTCAGCAGC |
| | | | | TTCATATCCAGACCTTGAAGAAATGAACGCAAGAACTACTCTTCAG |
| | | | | GCCAAAAAAAGTGCAGAATTTATAGAAACCGATATGTCTGTTGGGG |
| | | | | GAGATGTTTATCCAGTAGAGCAAGGAGATGACTTTCCATTTAATCA |
| | | | | AGTTCTTGATGCACACTGGGGTGTTCTTAACGAAGAAGATACAAGC |
| | | | | GGGCTTCGGTCACACACTTTATCGATGGTTGTAAATGACTCTCCCGG |
| | | | | AGTTTTAAATATAGTAACAGGAGTTTTTGCTCGTAGAGGTTACAATA |
| | | | | TTCAGAGTCTAGCAGTTGGTCATGCAGAAGTTGAAGGGCGATCCCG |
| | | | | CATCACAACTGTCGTTCCCGGTACAAATGAGTCTATCAGCAAGTTGG |
| | | | | TTCAACAACTATACAAGTTGATAGATGTTCATGAT |
| 1743 | Conyza | gDNA | 8066 | AAAGTGTAAATTAATTTAGACATGCGTTGATTTAATTACTTTTGATA |
| | canadensis | Contig | | AATTGAGAGTTGTAATTGGTCAATTGAGATTAGTTCAATTATCTTTT |
| | | | | AGTATATAGTAAGATTAAGATTAAGATAAGATAAGATTAAGATTTT |
| | | | | AGCATCCTGATTAGAAATTATGTATAACTATGTTATCTCTTCTCCGAA |
| | | | | GTTAATTTACCTAATTATTAGTATTGTTACATTACATTACCAAAGTTT |
| | | | | GATAAATTGGTAAATATAATTTGGAGTAAGGATTAAGGAATGAGCT |
| | | | | AAACACTAACGGGCCACTGGCCAAATTATTTTTTCTTAGCTTCTATAT |
| | | | | ATGGACCAAAAAGTTTACTCATTTTGGGCTACTTCAAATGAGCCACA |
| | | | | TGCAAAACCAAAAAAAAAAAAAAAACTTAAGAAAATAAAAAATAAA |
| | | | | TTGAGCCAACCTTATTTATGTCTTTTTAAATTATTATTTTTTTTTGGTT |
| | | | | TTGATTTTTTATCCCTCTCGCAAAATAGGTATAGGTATTTGACCTAAG |
| | | | | AATTTTTTAGCAAATATATCTAGAATGAAGTCGTCGTGGTATTGCTT |
| | | | | TTAAATAGATATATTAGATATTAGAAACATTTTATCGTGATATCATCT |
| | | | | TTAAATCACTACTAGAAAAATAGCCTTTAATGACACCAAAATTATGA |
| | | | | CACACGCATTTACGATGATGCGCAATTTTTGGTGTCATAAAAAAACT |
| | | | | GTCATAACTTGTAAAATCTGAAGGGTTATGACACACAATTTTTGGTG |
| | | | | TCATAAAAAAATGTAGTGTAACGCGGAATACACTTACATTTGCGTAT |
| | | | | CATCCTCAAGTGTCGTCTTATAATTTTATTCCCCATATAAAAGCTTAT |
| | | | | AATTTTTTATTTCAATCAGATCGAAGCCTATACTTTTTTTTTTCAATCA |
| | | | | GATCCAGAACCCCAAAACCCTATAATTTTTCAATCGAAGCCCCAAAA |
| | | | | TCGATCGATTAATCAATCAATTCAATAATCACAAATTAATGGTAACG |
| | | | | ATTATTCCTTTCTTTACAAATTCAACAGTATTTCATTGATTCTTAAGGT |
| | | | | TTCAATATTTTGATTATAGGCAGCACAAGATTAAATCGAAGAACTTG |
| | | | | ATAGTTTGTTTGATTACACTCGTAGTTGTTTAGAAATCATTCCCCTCG |
| | | | | ATGACGGTTCGTATATATACAATTAATAGTTTATAGATTTGTTATTAT |
| | | | | ATTAATCAATTGAAAATAACTAAGGTTTCGTTATTATATTTATTTTCC |
| | | | | ATTAATTAGTAAAAATTCAGTGCGATGAATTTTTGAAATATGCGATT |
| | | | | AGATCTGAGTTTGCTTTTTTCTAGACAAGCAGGCTTCGACACACACA |
| | | | | CTTTATCGATGGTTATAAATGACTCTCCTGGAGTTTTAAATATAGTA |
| | | | | ACAAGAGTTTTTGCTCGTAGAGGTTACAATATTCAGGTATGTTTTTT |
| | | | | TTGTATCTGGGATATTTCATTAATTTGCTTCTTGAACATATGGAATTA |
| | | | | CCAGAGTTTTAAGTAGTTTGGTGTTTGCATCTTTGATTCAGAGTCTG |
| | | | | TCAGTTGGTCATGCAGAAGTTGAAGGGCGATCCCGCATCAAAACTG |
| | | | | TCGTTCCGGGTACAAATGAGTCAACCAACAAGTTGGTTCAACAAAT |
| | | | | ATACAAGTTCTATCGCCATTGACCTGTTGGAGATCCATAAGAAAGA |
| | | | | GATGGATCTAATTTCTTCAAATATAAGATTGGGAGAGAAAGGAGCA |
| | | | | TTTTCATGGACCACTTTATTATATGTTTCTTCCACAAGCACCATATTG |
| | | | | TAATTTGAACGATTGCATTTATCATTTTCTTCCATTTTTTTGTCACCAA |
| | | | | GTAATGTGTTATGTAAAAGATGCAGGTCTCGTACCTGAAAGGCATA |
| | | | | TATCGGACTTATTTTACACCATCTTCCAACCTTGCTCCAAACATCTAA |
| | | | | AACTAATGGGCAGCCGGTAAACAAGTGGTCACTTGATTCTTCATTCA |
| | | | | CATTGCAAAACGGGCATAGTAACGTGGTTGGAGAAATACCACGTTT |
| | | | | TGACAGCTGTAAGAGTGTTGGTAAGCGATCTAATCCTGCCCGCCAG |
| | | | | TTCATGATATTCACCTTTTTTTTGGATCCATTTGTTCCAACAAAAGTC |
| | | | | AACACCCGAGACATTATTATCTACGGCGTCAAGTTTCTCCCTTACCTC |
| | | | | AACCACTATATATGAATCAGATTCAAATGTTGCAAGTATGTCCACCC |
| | | | | CTAGTTTATCAAAATCCTGCTTAAGTTTGTGTACATTTCTCCAAAGAC |
| | | | | CTCCGATTCTATTTTTGAGTGGAACATCGAGCACGTTACCTGCTTCA |
| | | | | CCATGAATAGCAACCATCGTCTTTCTCCATAACCTATCTGGTTCAACA |
| | | | | TGAAAACGAAACCACCAACGTGCTAGCAAAGCCATATTTGCAACCT |
| | | | | TTAAAGAACCAATACCAAGGCCACCCCTATCCTTCTGTCTAACGACC |
| | | | | TCATTCCATGCAACCCAACTAATTTTTTTATGCTCTTCATTTTGAGCCC |
| | | | | AGAAAAACCTTCTCCACAGCCTCTCCAATTGATCATTTATTTTCATAG |
| | | | | GAGCTCAAAACAATGAAAAATAGTGTATGGTAGACTTGAGAGAAC |
| | | | | AGATTGAATTAACGTGACTCTTCCTCCCACCGATAAATTTTTGGCCTT |
| | | | | CCAAGAGGATAATCTTTTATTAAAAACATCAACTACTGGTTGCCAAT |
| | | | | GACAACATCGATTCATACTAACCCCTAGAGGAAAGACCAGATACTC |
| | | | | CAATGGAAAAGACCCTTGCTTGAAATGAAAATGCTCAGCTACTTGT |
| | | | | GCTACTGATTCAGTTTGAGTCCCAATCCCGATGATTGTTGATTTATT |
| | | | | AACATTCATTTTTAACCCAGAAATAATGTAGAAACCTCTGATGATTC |
| | | | | TGACTAATTTCAAAAAGTTTGAGATAGACCAGGAGCCCAATAGAAG |
| | | | | TACATCATCGGCATATAATAGGTGACTAATAATAGGGCCTTCATTGG |
| | | | | CAACTAAATCCCGTTGATCAGGTTTTTTGAGATTTCATCATTCAGAA |
| | | | | AACAAGTCAAAGGTTCCATGGCTAAAGTAAACATTACTGATGGTTT |
| | | | | GACAAGTCACCACCCTCCAAGGTCCAGTTCTTATAACTTGCGTAATT |
| | | | | CTCTTAATAGTTTGACAATATGTTATATTGTGAAATTCTCTTAATAGT |
| | | | | TTAACTAATAGGAGACGTAAATATCATATTACACCACCATTTTGTAG |
| | | | | CTCTTGAGCTTGTTCCTAGTTCACTTTCCAGCTCTTCAGGTTGCTTTT |
| | | | | GTAATTCATTTGACTGATGGTTGTTCTTCTAGTTAGGTGATAGATAG |
| | | | | GACCCAAAATAGGGTATAAGAATTGCCAAAGGATTATAAATAAAGA |
| | | | | TTTCACCTGATATGTTTATAATTTACAGTGATTGTATTATGTTTGTAC |
| | | | | TAATTATGATGTCTTGGGCTGAAATAGATAGGACATAGGCGTCGGG |
| | | | | AGTTGCAAAGGGTAAACCGAGGATTGCAGGGAGGAAAACCCTTGC |
| | | | | TGATATTAACAATATTCCACAGAGAGGTACTGCTTCAGGTGAAGTT |
| | | | | AACAAGTCACAGCCAAGTTCAGACGCTGCAAGACATATTATCGAGC |
| | | | | GACTTCAAAAGGTACATCTTTTTGGTTGGTCATCTTAAAGCTAAAAC |
| | | | | ATGTCACAGTTTTGTTAACATGATTCAAGTAAATATCATTTTTTTGAT |
| | | | | ACATAATATAGATAGATAGATATGTTGATTCAGTAATTGAAGTTGCT |
| | | | | CATGTATGTTATCCAGAAAAATGCAGGATTTCTGAAGCTTTTGATGG |
| | | | | ATAAAAAGTATCCATTGATTTTCTTTTACATTTCATGTTGCTATGAGA |
| | | | | ATCTGAAAACTTTAATTTGGCCTTATAAGTTAACTAATGCTTTTGCAG |
| | | | | TAAACTAATTGAATTAAGTGGAGTCGAGGTGTAAAAAGTGAGGGC |
| | | | | CACTTTGCAGATAGTGAAACAACCGAATTTGCAACTTGCTCAGTCAA |
| | | | | ACAGCCAGATTTAAGCAGTTAGTTTACTTGGCGTAGTTAATGATTGA |
| | | | | TATACTATTGAGGCATTCATTTGAATATGACTAGTATGTGATAATGT |
| | | | | TTCATTTTATGCTGCAGGAACTAAATTTAGTCAGGGAAATGGTAAGT |
| | | | | AGTCCGGAAGATGCTAGGCGTTTTGTCGTCCTCTTTGACATCTAAGT |
| | | | | TCTTCCATTGGTCATATTCATACCCCATGGTTTTCTTGTTTTATCCAGC |
| | | | | AAAATTCTTTAAAGCATGAGCTTGGTTGCAAAAGCGCCTTGATTATT |
| | | | | GCAAAAAATCTCGAGCTGGAGGTTAGTTTAGTGTAATCTTCTTCATC |
| | | | | GGTAGCACACTTGCATGCCCCTTGGTGTATTTCTCTTTATTTAATGCT |
| | | | | TGCTGTTTGAGTAAGCCTGGCAAAATGGGAGGGTCGGGCGAGTTT |
| | | | | GGGTAACGGGTCAAAGCGGGTGCGGGTCCAAACGGGTCATTTCAT |
| | | | | AAATGCGTGTCAAACGGGTGCGGGTCGGGTGCGTGTCAACCCGCA |
| | | | | TTTTTGGTTTTTTTTTAATGGTATTTTAACATGTTTATAATAAAGTTAC |
| | | | | AACTGCTTAAGCTTTTCCTATCAATCTACCATTTTTTTAGCTGTTAACT |
| | | | | CGTGACTTGTGTGTTGACTTGTTTTGTGAATTTTTGATCGCGACTTGT |
| | | | | ATCGTAAGAGTTATAAAATGAAAATTACATGACAAAATTATATAATG |
| | | | | TGTAAAAACATACATAAATTGATATTTTTAACAAAAATATATGATGC |
| | | | | ATGTGTAAAAATTACATGGAAAAAATATTACTTAGATTTATTTTTAA |
| | | | | ATTTAATATATATTGGATCAAAGAAATGAAAGAATGCATCAATGAA |
| | | | | AAAATGACAAAAATAAATAATAATAATAATTATATTGATAATAATAA |
| | | | | TTATTATAATAATATATATTTAAGTGAATCTGATTAAAAGTTCTTTTC |
| | | | | GTTGATGTGAGAATTGAAAATAATTAATTAAATAAGGGGTGAAATA |
| | | | | ATAATTCATCTTAGTTAATAGATTGAACATCCATTAACAAGTTGAGT |
| | | | | TTACTCCAAATTGAAGGTGGGTAGGTCGTAGGTGTACTAGCGTACA |
| | | | | CTCTAACGGCAGTATGTACGACATTAACATAAATATATGAGTGATCA |
| | | | | CTCAAACTTTTGAAGACCTGCTCGACCCGCAAAGCTATATTGATAAT |
| | | | | TTAAGAGTTAAAAATGAGACCCGCTTCTATCCTTTTTGTAAGATCCA |
| | | | | GTTGTACCCAATAAACAAAATAAATTACCCAAACCTAATCTAATGAA |
| | | | | ATTGGGTATGGATTGCCAGGGCTATGTTTGAGTGTATGTTATCTCA |
| | | | | GGGAAAAGCGAAAACCAAGATATGTCAAATAAATGAAAATATTGTA |
| | | | | AGTACATAAACCTCAATAGTATTTTAGTTTTGTTCAAAATTTGTTGGT |
| | | | | TCAAGTTTTGTCTGCTACAATATAAGATGAAAATTTATGTTTCTATCT |
| | | | | TGTAGACAGGTAATGTAACTGAGCCAGAGGCGAAAAAAGTGATCC |
| | | | | AAGTAGCAGAAAGTGACAATGACAGAAGTAGAACGCAAAAATCTA |
| | | | | AATGTCAGAAATCTAAAACATGTGAATGATGATTTGGATGAAATTT |
| | | | | GTGAAGAATGGACAAAATATGTGACAAACTTCATTTTCCGGTCTTG |
| | | | | AATTCAATATAACAGTGGTTGTATGGATGTGATGAATTTTTATGGTT |
| | | | | AGTTTTAGAATTTTTAAATAACTTGTGGTTGTATGAAATACATTGTTT |
| | | | | AGATATATATAAAATGCGTTTTTTTGCTATTTGTGTATTTATTTGTAT |
| | | | | TATGAGATTAACTGTTATTTAATGTGTAGAGATATTAATAATTTTATA |
| | | | | AAAATATTTTTTTTTATAAAAAATTTGAACTATGACACGCAAAAAAG |
| | | | | GCGACATAAATTAGTGTCATTAAAGTGTGTCGTAAAAGAGTGTCAT |
| | | | | AAAAGCGTGTCATAAATTTATGACGTGCCAAATGCGTGTCATAAAG |
| | | | | ATTTATGACGGGTCGATTTATGACACCAAAAATCGTGTCATCTGACC |
| | | | | CCTTTTATGACACCAAATCGGTGTCGTTAAAGGTGTTTTTTTTCTAGT |
| | | | | ATCACTTTTAAATAGATATATTAGATATTAGAAACATTTTATCGTGAT |
| | | | | ATTGTCTTTAAATAATTTAATGACATGATAAACTTTTGATCAAACATA |
| | | | | TTTATGATAGAGTGATCTTCATCTTTCTTTTGAATTCACCAGTCATTC |
| | | | | GATGGGTTTCAAGGGGATCCTCGACTTTTCCACCTTTTTTTATCCTAA |
| | | | | ATGTTTGTCTACTCAAGAACAAATCAAAGACCAACTATCTATATCGG |
| | | | | GAGTAGCAATCCTCTATAGGAAAGGCCTTACCGTTTGCATACCCTAA |
| | | | | AGTCGTCGTTACGCACCAGTAGTCGATCTGAGTGTTATCGTCTACTC |
| | | | | TAGAAGGTAATCGACCAAATATATTTCTTTATGAAGAAAACATTTAC |
| | | | | GACAATCAAGTCGTTAGTCGTATGCAGTAACAAACTCCAATAGTGT |
| | | | | GCGTTTTGTTTTCATTTTCCTCTCTTAACCATATCTTAAGTTAAAGAA |
| | | | | AAATTATTAATCTTCTTAAAGCTTTTCCGAAAATCTCTAAAATTTGAC |
| | | | | ATGTAGATTTCATTAATTTTATTACTTAATCTCATTTTCAGATTATATT |
| | | | | ATTATCATAATCTTATCTCTACTACCTAATAAAATAAATCACACCCCC |
| | | | | CACAATAAATGTTTTCTGCCTTTAAAATTACTTATTTACCCTTGGACT |
| | | | | TACCTTTTTTATTTTTTTCTTTTAATATTTAATATTAATACAAAAAACT |
| | | | | GATTTCGACAAATAAAGTGGTCACCCATGTTTCTTTAGTATCTACGG |
| | | | | TTTAAGATTAATGTTGGTGGATCAATTATTTTTATTAAAGTTACAATT |
| | | | | TTCAATTTTAATAATTAAGATGTCCTTTATCTTTCATTTGGTAAGATT |
| | | | | AATGGATAAGGAAAAGTTTTATTTATAAAACATCTGGGCATAAAAT |
| | | | | ATTCTAGACCGCCTTCCCCACCACTGTCGCCGCATTCAGGGACGGTT |
| | | | | TTAGCATGGGGGCATTGGGGGCAAATGCCCCCATTCTAATTTTGGT |
| | | | | ACAATGTAATTTTTTTTATATAAATTCTAAAAAACTTAGTCTAAACCG |
| | | | | AGATTACATATAAATGTTTCTTTCTTTGTCATAAGTCTCTTTTTTGGA |
| | | | | TGTATATATATAGTATTACCTTGTCAAACTCGACAATAATACTGTGT |
| | | | | ATTTAGTTCCCCGTCTTCATCATGATCATCATCTTCTTCATTTCAAAG |
| | | | | GCTCATTTTCGAAGTATTTAGATGTAGAGAGTGATGAAGTGTAAGG |
| | | | | GGTTTGTGTTTATGATTCAGAGTGTTTGAATGTAAAATTTCAAAGAT |
| | | | | TTTGATGGTGATGGTGGTTGTTGCGGTGGTGGATCTCGTGGTGGTT |
| | | | | GTTGTGGTTGTTTCACATGTTGTGAAGGGAGGTATATTTGATTGTTG |
| | | | | AATTTACAGATAAACTGATAAAGTGAGATTGTATATGGAAGACTAA |
| | | | | AATCATTACTCGTATTTACTAATTTACCATCTATCTAGACTTGTCTTTA |
| | | | | TCAAATGGTGAAGTGTATATTGGAAAATTCCATCAATATTTTATTAG |
| | | | | TTACACCACCTACACACATTTTATAGATTCAAATATTATTGTAATTTT |
| | | | | TTTAATGTAATATTCCGTATATTTATATATATAAGTTTGATTTGATTT |
| | | | | GTGAAAAATTAATGTAGGTAAGAGTACAATATTTGAAATAAAATTG |
| | | | | TCTACGCGTGAAACAAAATTCTTTTAGGGTCCATTTTCTATCCTCGA |
| | | | | GACGGGTTTTAAAAAATTTTTATTTTATCTTTTTAGAAACGGTTTTAA |
| | | | | GAAATATAATAAAGACTCTTAAATTTGCCCCTTCTTGGAAAAAACTC |
| | | | | TGGCTCCGTCACTGACTGCATTGCGCGGGTACCATACTCGTAATTAA |
| | | | | AGATGATTTTTAGGTTGATTTGTTAGCTGTTTTTTTAAGTTAATATAC |
| | | | | TTATAGCTTTAGTTTCGTCGTAGCGAAAATGTGTTGCTTGTTCCACC |
| | | | | CAAAGCTATTTGCAAGTGGCTGGCTCATGTATGTGTTTCGGTATGTG |
| | | | | GGTGTATTTTTGATAATACTGAAATGGTACCTCACGTTTGTGCCATA |
| | | | | TTACTGGTTTTATACATTTTCGTTTGAAATTACGCTGATCATACCCAA |
| | | | | CGTATGC |
| 1744 | Conyza | gDNA | 7484 | TTTGCACAATGTAGCAGTGGTGGTGGTAATGATAGCGGCAGTAGG |
| | canadensis | Contig | | GACAATGATGGAAGCGACGGTGGTGGTAAATGTAAAAGTATTTGA |
| | | | | TGTTAAATGTGGTATTATAGTTATTTTAAGTGTTGGAGGATCTATAC |
| | | | | CTTAAATTATTACATTAAGGATATTATAGGAGTATATTAGATCGAGA |
| | | | | TGTTTAAATTAGTAAATAAAGAAGAAAAATATTTTTGATTTTTTAAA |
| | | | | TGTAGAAAGTTTAAGGAAAAAGAATTTATTTTATTAGATAATATAGA |
| | | | | TTATAACTAGTTGAATCGTTTTATAAAACCCGACTGCTTTGATTAAA |
| | | | | ATATCATTGGTTATAACTTTTAAACATAGGCCTCGTGTCACAATCCA |
| | | | | GCTTAAAGTAAATTTTGACAACCTTAAATCAAAAGCTCCTCAATATA |
| | | | | TAATATAACCAAAAAAACTCAACAAATAAATAAATAGATTAATATAT |
| | | | | ATACCTCAACAATGGCAGCCGCCGCCGTGACTACTTACTCTGCCACC |
| | | | | GCATTTCGTACTCGAACACCACCACCACCACCACCACCGTCTGTCAT |
| | | | | CCCCAATCAACTATCATATGCCTCCAAAAAACCCAACAGCAAATTTG |
| | | | | ACCTCAAAAAACTCTCTGTCAAAGCCTCCACCACTGGCTCTGTTCCC |
| | | | | GCCCCTGTTGACTCACCTTCATCCAATTCGTCTAGACCCACGTCAGC |
| | | | | CCCACCTCCACTCCCCCTTTTTTTATTATTATTAATTGTTTAGATTCAT |
| | | | | TTTTGCTAATCATATATATATATAGATTTCTAGTCATATATCTATGTTT |
| | | | | AGTCTTGTTATTTTATGCTGCATTTCGATGGGGACAATGGTAAAAAC |
| | | | | AATATTTACATTGGTAATGTGTGTGTATAGTTGTAAAATTTGCATGT |
| | | | | TTGTTTTGTTTGTGTTTTCAGTCATTATGCAACTTAGTATTCTTAAATT |
| | | | | TATGTTTTTTTCTTTTCAAATGAGATTTTGTTACGGATTGATCAGTGT |
| | | | | AGATGAGTGAAGTGTTAGCCTAAGGGAATAGGGATAGGGAATCTC |
| | | | | GATGTATCGGTTGCAATAGTTATTCTTCTAGTGACCCACAAAATAAT |
| | | | | TTGAAGAATGTTTGTCTAGTGGGGCGTATGGGGTTACCTTGCTATC |
| | | | | GAGGTCTAGGGTGAGAATCAAACTATAAGTATAGAAGGTTAAGAT |
| | | | | AGAGCTTAAGCCTTAAGGTGTGAGTGAGTGAAGAATAAAGCATATT |
| | | | | TTGAAGATGATGGTGATCTTTGGTTGAATTGTTGGATATGTAGTCAT |
| | | | | AACTAGTGGGGTTGCTGCCTGAGCCTGCATCGTGTGTCGGCTACTA |
| | | | | GTATGGATTCTGGGACGACTACGGATTGGGCCCCAGATACACAGTA |
| | | | | TTCCCTTTGTCAGGATTACACTTGGAGCACAATGCATAGTCTAGGTT |
| | | | | AATGATGACAGAGTTGCCACCTACTCTTGAAATCTATAGTACACTTC |
| | | | | AAGTTGACGTTATATCAGAGAGCTACTTAGGGTGTTCTATGTTATTT |
| | | | | AACCTCTCTTGTTGATATTCTCTGGGGTGTTTTTTGGGTATTTGATTG |
| | | | | CTCTTGGATTTCGTTGATTCCCTTTTAAGTGATCCTGGCTTTTGATTT |
| | | | | CCCCTATGAGGTCGTTGATACCCACTGAAGCTTTTTTGGTTATTGTC |
| | | | | CCATATTAAGTTTTTTTGGGGGCTTATTGGTGGTTTGATATTTGATC |
| | | | | ACATGGTATTGTATTTTTTTTCTTCAAATATTGAAGTTATGAGTGTTG |
| | | | | TTTCTACCATGTGATAAATATCTAAGTCTATGCTTAGTAACTACATTT |
| | | | | GATACTTAAGTTAGAACTGGATATTGAACTTTTACTAACCGATATGG |
| | | | | TTAACTAAACACCATAGGCATTGATATCTGATGCCATTTAATTGCGT |
| | | | | GTAGTTATTTAAATTATTTGCTTTCCTTATTTATACTTTGTCATTTGCC |
| | | | | TTCATATATTCTTATGAAAGGTTTACATATTAATTTCTCGATATATCTT |
| | | | | TATAAATAAAGTTTAATGTAACACAGGGTGAGGCGCCACACTATTT |
| | | | | CAGTATTTGTTGGTGATGAGAGTGGAATGATAAATCGGATTGCAGG |
| | | | | AGTTTTCGCAAGGAGGGGATATAATATCGAGTCTCTTGCTGTTGGT |
| | | | | TTAAACAAGGACAAAGCTCTTTTCACTATAGTTGTATCTGGAACCGA |
| | | | | AAGGGTGTTGCAGCAAGTTATGAAACAGCTTCTAAAGCTTGTTAAT |
| | | | | GTTTTGAAGGTTGACTTCTGTTTCAAGTTTAAACCATCGATCCATTAT |
| | | | | GGTTATTTTCTTTTTTAAGTTTCTCGAAGTTGACTACCATGAACAGGT |
| | | | | CGAAGATATCTCAAAGGAGCCACAAGTAGAACGTGAGTTGATGCTT |
| | | | | ATCAAGATCAATGCAGATCCAAGTTACCGTATGGAGGTAATTATGC |
| | | | | AATGTTAATCATGTTTGGGTTTTTGGCTGTTATTCATAACATACTACA |
| | | | | TTATTCTGATTCATGGAGACGAACATATTTCCATGACGGGGGTGTTT |
| | | | | TAGATATGCCATTTAAATTGTTCATATGATTAGTGTGACCTTTAGTAC |
| | | | | TGATAACATAAGTGTTTGGATAAACAGATTTTGCTTATGACTGTATT |
| | | | | ATGATTATCTAGTGGACAAGTAAAATGTGTGAGAATATGTTATCTCA |
| | | | | CCATATTCTTATGGTTTTGATTTTCAGGTTAAGTGGTTAGTGGACAT |
| | | | | CTTTAGAGCTAGAATCGTTGATATCTCACAGGACTCGCTAACAATCG |
| | | | | AGGTGATGGAAGTTGAAATTTTACTTTAACCTTTAAAGTGTTTACTG |
| | | | | TTTTTTCAGGCACAACTGATCTGGACTCAGTTTTGGGTCTTTATGTAT |
| | | | | TGTAATTTACAAAAGTATAATGCTCTATAGAGGACCTGTGTTAGTTT |
| | | | | GTAATTTTTCAATGACCTTTTTGATAAATATGAAATTCGTACACACTT |
| | | | | TGACTCTTTGGAATTTAGGTAACTGGTGACCCAGGGAAAATGGTTG |
| | | | | CAGTGCAAAGAAACCTGAGTAAATTTGGGATTCGAGAAGTTGCAA |
| | | | | GGACTGGAAAAGTAAGTTATTATTAAGTCTCTATTTTTTCCTATTCTT |
| | | | | CTTTCATATTGATTGTTAAATTCTGTATGCCTTAGATTGCTTTGAGAA |
| | | | | GAGAAAAAATGGGCGAGGATGCTCCTTTCTGGCGTTTTTCAGCAGC |
| | | | | TTCATATCCAGACCTTGAAGAAATGAACGCAAGAACTACTCTTCAG |
| | | | | GCCAAAAAAAGTGCAGAATTTATAGAAACCGATATGTCTGTTGGGG |
| | | | | TAAGTTGCTTTCTCTTTTTGACTGACGCTAACCTGGGTTGTAGAGGT |
| | | | | TGCAAATTAGTTGGGAAATTGGGTCAAAGCGGAATTTTTTTTTGCG |
| | | | | GATCGGCTAATGTTGACCTGAATTGGTGTTTGTCCCAAGTTCAGTTT |
| | | | | GATCTTTTATACATAATTAGTTTGTAAAACATAACTAAACTTTTTATA |
| | | | | TATGTAAATGCAACTATACGGCATTGCTTTTCAACAAAATGATATAG |
| | | | | GAGGCACCATTGAAAAGAAAGATTTTAGTGGTTTTATGCTTAAAAC |
| | | | | TACATTTGGCAACTTTCAGCCGAAGCTCTTTGTTTTTAAACTTATTTG |
| | | | | CTTTACTCGTTTGACCCGTTATAATATAAATTAGTCTGGTTCGACGAT |
| | | | | TATCTATCTTGCACGATGTAGTTTAGACTTGAATACTTCATACACAG |
| | | | | ATGGTGAATGAGGTGGTATTTCTTATCTGTATTAAATTATATTTGTG |
| | | | | AAGGGAGATGTTTATCCAGTAGAGCAAGGAGATGACTTTCCATTTA |
| | | | | ATCAAGTTCTTGATGCACACTGGGGTGTTCTTAACGAAGAAGATGT |
| | | | | AAGAACTTGATATCACTTCTCATATTTCACAAATGTCACTGAGGTGC |
| | | | | ATTGAAGTATCAGTAATTTTTAGTTTGATGTTTCTAGACAAGCGGGC |
| | | | | TTCGGTCACACACTTTATCGATGGTTGTAAATGACTCTCCCGGAGTT |
| | | | | TTAAATATAGTAACAGGAGTTTTTGCTCGTAGAGGTTACAATATTCA |
| | | | | GGTATGTTTTTTGTATTCCGGATATTTCATTAATTTGCTTCCTGAACA |
| | | | | TATGGAAGTACCAGAGGTTCAAGTAGTTTGGTGTTTGCATTTTTGAT |
| | | | | TCAGAGTCTAGCAGTTGGTCATGCAGAAGTTGAAGGGCGATCCCGC |
| | | | | ATCACAACTGTCGTTCCCGGTACAAATGAGTCTATCAGCAAGTTGGT |
| | | | | TCAACAACTATACAAGTTGATAGATGTTCATGATGTGAGTATATATT |
| | | | | GTAGTTGGAAAATGCTTCATATGTTAAGTTTATTATGAATGAGCAGT |
| | | | | ACTACTAGTCTCATTTTCCGCCCTCCAAGGTCCATTTGTAGTTGTTTA |
| | | | | CGACCCAGTTCTTATGGAAAGGTAGATTTGGGCTGTGCTGGTAAAA |
| | | | | TTGAAAGGTTTAACTAATAAGGAAATGGGTCAAGTGGTTCGAACAG |
| | | | | GTTGAAAGTTTCCCAGAGAGTGATACTCGTATAATTATTGTAATTCT |
| | | | | CTTGATCGCTTTTATCACCAGTCTGTAAAAAACGAAAAAGTTTGACA |
| | | | | AGATGTGTTTCACTTCAAATAGGTTATAGAAAAATAGATTTATTAAA |
| | | | | ATAATGTAATTTTTGTTATTTTATTTGACCCAAAAGGACTTTGAATAG |
| | | | | TACCAAAAGGACTCTTCATTGACCCAGTACCCACCTGTCTGTTTTGTC |
| | | | | ACCCCTACCCATTTGGTTGCTATTGATACTACTGAGGAGATATAATT |
| | | | | ATTCTATTACACCACCATTTTGTAGCTCTTGATCTGGTTTCTAGTTCA |
| | | | | CATTCCAGCTCTTGAGGCTGCTTTTCTAATTCATTTGACTGATACTTG |
| | | | | TTTTTCTGTTTAGGTGATAGACCTAACATCCGTGCCATTTGCTGAGC |
| | | | | GAGAGTTAATGCTGATTAAAGTTGCCGTCAATTCCACAGCTCGACG |
| | | | | CAGTGTTCTTGATATTGCAAGCATATTTAGAGCTAAAGCCGTAGATG |
| | | | | TATCAGATCACACAATTACACTCGAGGTGAAATTTAAATATAATTCA |
| | | | | CTTGCATATTACACGTGTTGACTGAAATTTCATGATCCTACTATTGAA |
| | | | | CGTTACAAAATTTCATGATCCTTACCTTTATTTTCACAGTTTTTCTGTT |
| | | | | TTTCTTTCCCCGTTGGGTTGTTTCAGCAATAAATTGTTTTTCTTGTATT |
| | | | | TTCCAGGTCACTGGAGATGTAAATAAGATGGTAGCATTACAGAGAC |
| | | | | TGTTAGAGCCATGTGGAATTTGCGAGGTTTGTTTGTGAAATCTGTTA |
| | | | | TTTATAATATTTCTATTTGAACATGTCACCTTCCAGTTTAAAACCCAC |
| | | | | AGCTTGGTTTGCTCATTTGGAACCATGAACAATCTCATTGTTCGTGA |
| | | | | CCAAACTACAGTTCTAGAACTGAACTAGAGTGGTCAGTCTTTTTGTG |
| | | | | TTGCAGTCCTATTTACTTAATCCTTGATCATTATTTGTAGGTTGCAAG |
| | | | | AACAGGGAGGGTGGCACTGTCTCGTGAGTCTGGTGTTGATTCATCT |
| | | | | TACCTTCGTGGCTATTCCTATCCCGTTTGATTCAGTCATTTAGAGTGT |
| | | | | CTTTTTGTTTTTGGTTGCTCGGAGTATAGAATTATATATTTTCAATGA |
| | | | | TTGAATATCTACAAGTCGTTTTAGCATGTATGTTTTAATAAAAGCTG |
| | | | | CTGTTTTGAATGAGGGTTTGGTATGAACCCAATTTGAAAGATTTTGT |
| | | | | TTTTTCTTTAAATAGGCGCCTAAGCTCAACAAGGGCACATGGTTAGA |
| | | | | TTGGTTGAACTAGTTTTTTAGGGAAACATGTTCCTCATCAAGTTCTA |
| | | | | ATTTGCAAGATTGACCGAACCCATGTCCCTGCCATTTTTTCCGTTCAT |
| | | | | GTCTTGAGCCGAAGTTATAACTCAGGTTTAAGCACACCCTCATTAAA |
| | | | | ACTCTTCTAATAAATTGAATGGAATTTTTAGTCCTCAAATATCCTCGC |
| | | | | GAGATTTTAGCTCGTGATTTATGCTGCCCACAGAGAATATGTGGTTT |
| | | | | TTGATGACCCTATTTTTCAGTTAATTTCCAAACATCATCCACCAATTC |
| | | | | CACTCTTTTTGGGAGTCAAAATTTATTGACAAAATTAGAAAGAGAG |
| | | | | AAACAACCAAAAATCATAGAGGAATTAAAAATAAAAATATGAAAAA |
| | | | | AGATGATCTAACATGAGAATGGACATCGAACATGGAATATAATGTT |
| | | | | AACAAATATCCACAAAGACTGAATTAATCAGAGAATGTGGCTTCAG |
| | | | | AAAAGCGGAAGTAGTTTGCAGGGTGGTGATCGAATGCAGAGGAAG |
| | | | | GGAACTGGAACCGCCATGGTTGCGGTGGCTATAGACAAGGACAAA |
| | | | | TACAGCCAGCAAGCGCTTAAATGGTCCATTGAACATCTTCTTACCCG |
| | | | | TGGACAAACCGTTGTCCTCATTCATGTCATCCGGAAAACCCCTCCTA |
| | | | | CAGGCAATATTTGTATTCTGTCAAACGATTCAGATATTAACATTGTTT |
| | | | | ATAACAACTAATTGAAACAGAGAGAGAGAGAGAGAGAGAGAGAG |
| | | | | AGAGAGAACCTTATAATCCCATATCAATTTCAACTATTTGACCAATA |
| | | | | TATAACCCCTAACACGATTGTTGAGTCACATAATCCTTGAAACAAGA |
| | | | | TAAAACAATTAGACTGCGTTAAGTTACAGAAAACGTTTTCTAATTTT |
| | | | | CCACTTAGAATTCATAAGAAAATGGAAGGGGTTTTTTAGTTCTAGA |
| | | | | AACTTAAACTTCTGAAAATGCGTTTAGTTGTAAGTGGAAAACGTAA |
| | | | | GGAGTTTTTCATTTTTAAATAACAGATGTGCCAATATGAATGTAGGG |
| | | | | GGAGGGATTGATGCATCTGCTGCAAACAAGCAACAAACTGAGAAG |
| | | | | CAAACAAGAGATTTGTTCCTCACGTTCCATTGCTTCTGTACGCGTAA |
| | | | | AGAAGTACGTGTTTTACTACTATCTGTCTTGTTGGGTATCTTTTGCAT |
| | | | | GGTTTTGCTAAAATTGTTGCACTGCAGATACAGTGTTACGATGTTAT |
| | | | | ACTTGAAGATGCAAATATAGCAAAAGCACTCACGGAATATGCATCT |
| | | | | TATGCTGCCATAGAATATTTGGTCCTTGGTGCCTCATCACGGCATGG |
| | | | | TTTCATCAGGTCATTTATTCAAATGAAATTTTTGCAAAACCTTTAAAT |
| | | | | GTTTTAACGATTATCACCTTTGAGTTAATATTGTAAGCATTTCTATAA |
| | | | | TGATAGCTGAATGCTTGGACTAATGAGTAACGATTAAAACATTTATA |
| | | | | AACAGATTTCGGAGTTCTGATGTGCCAAGCCAAGTAATGAAATCGA |
| | | | | TACCAGATTTCTGCACTGTTTATGTCATCTCAAAAGGAAAGATATCC |
| | | | | TCAGCAAAGAAATCTTCACGAACAGTCCCGTTTCCATCTCCTATACG |
| | | | | TGAGAAAATAGAAGAACAATCTAACACCAACTTTGAAGCAAGTGTA |
| | | | | CGCCGCAAGAATAGTTTTAACATTAAAAGTCAGTCATTTATTTTCAG |
| | | | | TTTAAATACAATACATTCCCAAAAGTTATCTTGTTTTTCATGACTCAA |
| | | | | AGAACTTGTGAACAGAACCAGAAATCGCGCCAGAGAAGCCACTACT |
| | | | | TCCGGCACCAGAAGA |
| 1745 | Conyza | gDNA | 7382 | CTATACCTTAAATTATTACATTAAGGGTATTATAGGAGTATATTAGA |
| | canadensis | Contig | | TCGAGATGTTTAAATTAGTAAATAAAGAAGAAAAATATTTTTGATTT |
| | | | | TTTAAATGTAGAAAGTTTAAGGAAAAAGAATTTATTTTATTAGATAA |
| | | | | TATAGATTATAACTAGTTGAATCGTTTTATAAAACCCGACTGCTTTG |
| | | | | ATTAAAATATCATTGGTTATAACTTTTAAACATAGGCCTCGTGTCAC |
| | | | | AATCCAGCTTAAAGTAAATTTTGACAACCTTAAATCAAAAGCTCCTC |
| | | | | AATATATAATATAACCAAAAAAACTCAACAAATAAATAAATAGATTA |
| | | | | ATATATATACCTCAACAATGGCAGCCGCCGCCGTGACTACTTACTCT |
| | | | | GCCACCGCATTTCGTACTCGAACACCACCACCACCACCACCGTCTGC |
| | | | | ATCCCCAATCAACTATCATATGCCTCAAATGGCATACCTTGTACCTTC |
| | | | | ACCACCATTTCTTCAAAACCCAACAGCAAATTTGACCTCAAAAAACT |
| | | | | CTCTGTCAAAGCCTCCACCACTGGCTCTGTTCCCGCCCCTGTTGACTC |
| | | | | ACCTTCATCCAATTCGTCTAGACCCACGTCAGCCCCACCTCCACTCCC |
| | | | | CCTTTTTTTATTATTATTAATTGTTTAGATTCATTTTTGCTAATCATAT |
| | | | | ATATATATAGATTTCTAGTCATATATCTATGTTTAGTCTTGTTATTTTA |
| | | | | TGCTGCATTTCGATGGGGACAATGGTAAAAACAATATTTACATTGG |
| | | | | TAATGTGTGTGTATAGTTGTAACATTTGCATGTTTGTTTTGTTTGTGT |
| | | | | TTTCAGTCATTATGCAACTTAGTATTCTTAAATTTATGTTTTTTTCTTT |
| | | | | TCAAATGAGGTTTTGTTACGGATTGATCAGTGTAGATGAGTGAAGT |
| | | | | GTTAGCCTAAGGGAATAGGGATAGGGAATCTCGATGTATCGGTTGC |
| | | | | AATAGTTATTCTTCTAGTGACCCACAAAATAATTTGAAGAATGTTTG |
| | | | | TCTAGTGGGGCGTATGGGGTTACCTTGCTATCGAGGTCTAGGGTGA |
| | | | | GAATCAAACTATAAGTATAGAAGGTTAAGATAGAGCTTAAGCCTTA |
| | | | | AGGTGTGAGTGAGTGAAGAATAAAGCATATTTTGAAGATGATGGT |
| | | | | GATCTTTGGTTGAATTGTTGGATATGTAGTCATAACTAGTGGGGTT |
| | | | | GCTGCCTGAGCCTGCATCGTGTGTCGGCTACTAGTATGGATTCTGG |
| | | | | GACGACTACGGATTGGGCCCCAGATACACAGTATTCCCTTTGTCAG |
| | | | | GATTACACTTGGAGCACAATGCATAGTCTAGGTTAATGATGACAGA |
| | | | | GTTGCCACCTACTCTTGAAATCTATAGTACACTTCAAGTTGACGTTAT |
| | | | | ATCAGAGAGCTACTTAGGGTGTTCTATGTTATTTAACCTCTCTTGTT |
| | | | | GATATTCTCTGGGGTGTTTTTTGGGTATTTGATTGCTCTTGGATTTCG |
| | | | | TTGATTCCCTTTTAAGTGATCCTGGCTTTTGATTTCCCCTATGAGGTC |
| | | | | GTTGATACCCACTGAAGCTTTTTTGGTTATTGTCCCATATTAAGTTTT |
| | | | | TTTGGGGGCTTATTGGTGGTTTGATATTTGATCACATGGTATTGTAT |
| | | | | TTTTTTTCTTCAAATATTGAAGTTATGAGTGTTGTTTCTACCATGTGA |
| | | | | TAAATATCTAAGTCTATGCTTAGTAACTACATTTGATACTTAAGTTAG |
| | | | | AACTGGATATTGAACTTTTACTAACCGATATGGTTAACTAAACACCA |
| | | | | TAGGCATTGATATCTGATGCCATTTAATTGCGTGTAGTTATTTAAATT |
| | | | | ATTTGCTTTCCTTATTTATACTTTGTCATTTGCCTTCATATATTCTTAT |
| | | | | GAAAGGTTTACATATTAATTTCTCGATATATCTTTATAAATAAAGTTT |
| | | | | AATGTAACACAGGGTGAGGCGCCACACTATTTCAGTATTTGTTGGT |
| | | | | GATGAGAGTGGAATGATAAATCGGATTGCAGGAGTTTTCGCAAGG |
| | | | | AGGGGATATAATATCGAGTCTCTTGCTGTTGGTTTAAACAAGGACA |
| | | | | AAGCTCTTTTCACTATAGTTGTATCTGGAACCGAAAGGGTGTTGCAG |
| | | | | CAAGTTATGAAACAGCTTCTAAAGCTTGTTAATGTTTTGAAGGTTGA |
| | | | | CTTCTGTTTCAAGTTTAAACCATCGATCCATTATGGTTATTTTCTTTTT |
| | | | | TAAGTTTCTCGAAGTTGACTACCATGAACAGGTCGAAGATATCTCAA |
| | | | | AGGAGCCACAAGTAGAACGTGAGTTGATGCTTATCAAGATCAATGC |
| | | | | AGATCCAAGTTACCGTATGGAGGTAATTATGCAATGTTAATCATGTT |
| | | | | TGGGTTTTTGGCTGTTATTCATAACATACTACATTATTCTGATTCATG |
| | | | | GAGACGAACATATTTCCATGACGGGGGTGTTTTAGATATGCCATTT |
| | | | | AAATTGTTCATATGATTAGTGTGACCTTTAGTACTGATAACATAAGT |
| | | | | GTTTGGATAAACAGATTTTGCTTATGACTGTATTATGATTATCTAGT |
| | | | | GGACAAGTAAAATGTGTGAGAATATGTTATCTCACCATATTCTTATG |
| | | | | GTTTTGATTTTCAGGTTAAGTGGTTAGTGGACATCTTTAGAGCTAGA |
| | | | | ATCGTTGATATCTCACAGGACTCGCTAACAATCGAGGTGATGGAAG |
| | | | | TTGAAATTTTACTTTAACCTTTAAAGTGTTTACTGTTTTTTCAGGCAC |
| | | | | AACTGATCTGGACTCAGTTTTGGGTCTTTATGTATTGTAATTTACAA |
| | | | | AAGTATAATGCTCTATAGAGGACCTGTGTTAGTTTGTAATTTTTCAA |
| | | | | TGACCTTTTTGATAAATATGAAATTCGTACACACTTTGACTCTTTGGA |
| | | | | ATTTAGGTAACTGGTGACCCAGGGAAAATGGTTGCAGTGCAAAGA |
| | | | | AACCTGAGTAAATTTGGGATTCGAGAAGTTGCAAGGACTGGAAAA |
| | | | | GTAAGTTATTATTAAGTCTCTATTTTTTCCTATTCTTCTTTCATATTGA |
| | | | | TTGTTAAATTCTGTATGCCTTAGATTGCTTTGAGAAGAGAAAAAATG |
| | | | | GGCGAGGATGCTCCTTTCTGGCGTTTTTCAGCAGCTTCATATCCAGA |
| | | | | CCTTGAAGAAATGAACGCAAGAACTACTCTTCAGGCCAAAAAAAGT |
| | | | | GCAGAATTTATAGAAACCGATATGTCTGTTGGGGTAAGTTGCTTTCT |
| | | | | CTTTTTGACTGACGCTAACCTGGGTTGTAGAGGTTGCAAATTAGTTG |
| | | | | GGAAATTGGGTCAAAGCGGAATTTTTTTTTGCGGATCGGCTAATGT |
| | | | | TGACCTGAATTGGTGTTTGTCCCAAGTTCAGTTTGATCTTTTATACAT |
| | | | | AATTAGTTTGTAAAACATAACTAAACTTTTTATATATGTAAATGCAA |
| | | | | CTATACGGCATTGCTTTTCAACAAAATGATGTAGGAGGCACCATTG |
| | | | | AAAAGAAAGATTTTAGTGGTTTTATGCTTAAAACTACATTTGGCAAC |
| | | | | TTTCAGCCGAAGCTCTTTGTTTTTAAACTTATTTGCTTTACTCGTTTG |
| | | | | ACCCGTTATAATATAAATTAGTCTGGTTCGACGATTATCTATCTTGCA |
| | | | | CGATGTAGTTTAGACTTGAATACTTCATACACAGATGGTGAATGAG |
| | | | | GTGGTATTTCTTATCTGTATTAAATTATATTTGTGAAGGGAGATGTT |
| | | | | TATCCAGTAGAGCAAGGAGATGACTTTCCATTTAATCAAGTTCTTGA |
| | | | | TGCACACTGGGGTGTTCTTAACGAAGAAGATGTAAGAACTTGATAT |
| | | | | CACTTCTCATATTTCACAAATGTCACTGAGGTGCATTGAAGTATCAG |
| | | | | TAATTTTTAGTTTGATGTTTCTAGACAAGCGGGCTTCGGTCACACAC |
| | | | | TTTATCGATGGTTGTAAATGACTCTCCCGGAGTTTTAAATATAGTAA |
| | | | | CAGGAGTTTTTGCTCGTAGAGGTTACAATATTCAGGTATGTTTTTTG |
| | | | | TATTCCGGATATTTCATTAATTTGCTTCCTGAACATATGGAAGTACCA |
| | | | | GAGGTTCAAGTAGTTTGGTGTTTGCATTTTTGATTCAGAGTCTAGCA |
| | | | | GTTGGTCATGCAGAAGTTGAAGGGCGATCCCGCATCACAACTGTCG |
| | | | | TTCCCGGTACAAATGAGTCTATCAGCAAGTTGGTTCAACAACTATAC |
| | | | | AAGTTGATAGATGTTCATGATGTGAGTATATATTGTAGTTGGAAAA |
| | | | | TGCTTCATATGTTAAGTTTATTATGAATGAGCAGTACTACTAGTCTC |
| | | | | ATTTTCCGCCCTCCAAGGTCCATTTGTAGTTGTTTACGACCCAGTTCT |
| | | | | TATGGAAAGGTAGATATGGGCTGTGCTGGTAAAATTGAAAGGTTTA |
| | | | | ACTAATAAGGAAACAGGTCAAGTGGTTCGAACAGGTTGAAAGTTTC |
| | | | | CCAGAGAGTGATACTCGTATAATTATTGTAATTCTCTTGATCGCTTTT |
| | | | | ATCACCAGTCTGTAAAAAACGAAAAAGTTTGACAAGATGTGTTTCA |
| | | | | CTTCAAATAGGTTATAGAAAAATAGATTTATTAAAATAATGTAATTT |
| | | | | TTGTTATTTTATTTGACCCAAAAGGACTTTGAATAGTACCAAAAGGA |
| | | | | CTCTTCATTGACCCAGTACCCACCTGTCTGTTTTGTCACCCCTACCCA |
| | | | | TTTGGTTGCTATTGATACTACTGAGGAGATATAATTATTCTATTACAC |
| | | | | CACCATTTTGTAGCTCTTGATCTGGTTTCTAGTTCACATTCCAGCTCT |
| | | | | TGAGGCTGCTTTTCTAATTCATTTGACTGATACTTGTTTTTCTGTTTA |
| | | | | GGTGATAGACCTAACATCCGTGCCATTTGCTGAGCGAGAGTTAATG |
| | | | | CTGATTAAAGTTGCCGTCAATTCCACAGCTCGACGCAGTGTTCTTGA |
| | | | | TATTGCAAGCATATTTAGAGCTAAAGCCGTAGATGTATCAGATCAC |
| | | | | ACAATTACACTCGAGGTGAAATTTAAATATAATTCACTTGCATATTA |
| | | | | CACGTGTTGACTGAAATTTCATGATCCTACTATTGAACGTTACAAAA |
| | | | | TTTCATGATCCTTACCTTTATTTTCACAGTTTTTCTGTTTTTCTTTCCCC |
| | | | | GTTGGGTTGTTTCAGCAATAAATTGTTTTTCTTGTATTTTCCAGGTCA |
| | | | | CTGGAGATGTAAATAAGATGGTAGCATTACAGAGACTGTTAGAGCC |
| | | | | ATGTGGAATTTGCGAGGTTTGTTTGTGAAATCTGTTATTTATAATAT |
| | | | | TTCTATTTGAACATGTCACCTTCCAGTTTAAAACCCACAGCTTGGTTT |
| | | | | GCTCATTTGGAACCATGAACAATCTCATTGTTCGTGACCAAACTACA |
| | | | | GTTCTAGATCTGAACTAGAGTGGTCAGTCTTTTTGTGTTGCAGTCCT |
| | | | | ATTTACTTAATCCTTGATCATTATTTGTAGGTTGCAAGAACAGGGAG |
| | | | | GGTGGCACTGTCTCGTGAGTCTGGTGTTGATTCATCTTACCTTCGTG |
| | | | | GCTATTCCTATCCCGTTTGATTCAGTCATTTAGAGTGTCTTTTTGTTTT |
| | | | | TGGTTGCTCGGAGTATAGAATTATATATTTTCAATGATTGAATATCT |
| | | | | ACAAGTCGTTTTAGCATGTATGTTTTAATCAAAGCTGCTGTTTTGAA |
| | | | | TGAGGGTTTGGTATGAACCCAATTTGAAAGATTTTGTTTTTTCTTTA |
| | | | | AATAGGCGCCTAAGCTCAACAAGGGCACATGGTTAGATTGGTTGAA |
| | | | | CTAGTTTTTTAGGGAAACATGTTCCTCATCAAGTTCTAATTTGCAAG |
| | | | | ATTGACCGAACCCATGTCCCTGCCATTTTTTCCGTTCATGTCTTGAGC |
| | | | | CGAAGTTATAACTCAGGTTTAAGCACACCCTCATTAAAACTCTTCTA |
| | | | | ATAAATTGAATGGAATTTTTAGTCCTCAAATATCCTCGCGAGATTTT |
| | | | | AGCTCGTGATTTATGCTGCCCACAGAGAATATGTGGTTTTTGATGAC |
| | | | | CCTATTTTTCAGTTAATTTCCAAACATCATCCACCAATTCCACTCTTTT |
| | | | | TGGGAGTCAAAATTTATTGACAAAATTAGAAAGAGAGAAACAACCA |
| | | | | AAAATCATAGAGGAATTAAAAATAAAAATATGAAAAAAGATGATCT |
| | | | | AACATGAGAATGGACATCGAACATGGAATATAATGTTAACAAATAT |
| | | | | CCACAAAGACTGAATTAATCAGAGAATGTGGCTTCAGAAAAGCGGA |
| | | | | AGTAGTTTGCAGGGTGGTGATCGAATGCAGAGGAAGGGAACTGGA |
| | | | | ACCGCCATGGTTGCGGTGGCTATAGACAAGGACAAATACAGCCAG |
| | | | | CAAGCGCTTAAATGGTCCATTGAACATCTTCTTACCCGTGGACAAAC |
| | | | | CGTTGTCCTCATTCATGTCATCCGGAAAACCCCTCCTACAGGCAATA |
| | | | | TTTGTATTCTGTCAAACGATTCAGATATTAACATTGTTTATAACAACT |
| | | | | AATTGAAAAAAAGAGAGAGAGAGAGAGAACCTTATAATCCCATATC |
| | | | | AATTTCAACTATTTGACCAATATATAACCCCTAACACGATTGTTGAG |
| | | | | TCACATAATCCTTGAAACAAGATAAAACAATTAGACTGCGTTAAGTT |
| | | | | ACAGAAAACGTTTTCTAATTTTCCACTTAGAATTCATAAGAAAATGG |
| | | | | AAGGGGTTTTTTAGTTCTAGAAACTTAAACTTCTGAAAATGCGTTTA |
| | | | | GTTGTAAGTGGAAAACGTAAGGAGTTTTTCATTTTTAAATAACAGAT |
| | | | | GTGCCAATATGAATGTAGGGGGAGGGATTGATGCATCTGCTGCAA |
| | | | | ACAAGCAACAAACTGAGAAGCAAACAAGAGATTTGTTCCTCACGTT |
| | | | | CCATTGCTTCTGTACGCGTAAAGAAGTACGTGTTTTACTACTATCTG |
| | | | | TCTTGTTGGGTATCTTTTGCATGGTTTTGCTAAAATTGTTGCACTGCA |
| | | | | GATACAGTGTTACGATGTTATACTTGAAGATGCAAATATAGCAAAA |
| | | | | GCACTCACGGAATATGCATCTTATGCTGCCATAGAATATTTGGTCCT |
| | | | | TGGTGCCTCATCACGGCATGGTTTCATCAGGTCATTTATTCAAATGA |
| | | | | AATTTTTGCAAAACCTTTAAATGTTTTAACGATTATCACCTTTGAGTT |
| | | | | AATATTGTAAGCATTTCTATAATGATAGCTGAATGCTTGGACTAATG |
| | | | | AGTAACGATTAAAACATTTATAAACAGATTTCGGAGTTCTGATGTGC |
| | | | | CAAGCCAAGTAATGAAATCGATACCAGATTTCTGCACTGTTTATGTC |
| | | | | ATCTCAAAAGGAAAGATATCCTCAGCAAAGAAATCTTCACGAACAG |
| | | | | TCCCGTTTCCATCTCCTATACGTGAGAAAATAGAAGAACAATCTAAC |
| | | | | ACCAACTTTGAAGCAAGTGTACGCCGCAAGAATAGTTTTAACATTA |
| | | | | AAAGTCAGTCATTTATTTTCAGTTTAAATACAATACATTCCCAAAAGT |
| | | | | TATCTTGTTTTTCATGACTCAAAGAACTTGTGAACAGAACCAGAAAT |
| | | | | CGCGCCAGAGAAGCCACTACTTCCGGCACCAGAAGACCCTGAATCA |
| | | | | ATTA |
| 1746 | Conyza | gDNA | 6631 | TTCCATATTTGGCATGATTTGGATTTTGAAAGGATAAGTTATAAATT |
| | canadensis | Contig | | TATCAATATATCCTTCAAACTTTTATATGAACTATAATAACTATGGGG |
| | | | | CTATAAATGTAATATTATGTATTTTTATCCTTTCTTATTCTTCTCTTTTA |
| | | | | ACTCAAGAATGCCTTTAAATCCTTTTATTCACTTTCTTTTCTTTTAAAT |
| | | | | GATAAAACTCAAGAATGCAATTTATAAATATAATTTTATATCCTTTCT |
| | | | | TCTCCTATTCAATCTTCTCTTTTATTCTCCTTTAATAAAACTCGAAAAT |
| | | | | ACAGTGTTAGGGTATTGCCAAAAGTCAAACCGGCAGAGTGCATATA |
| | | | | TATTGGAGGTGTTAGCCATTTTTATTTATAAAAAAAAAAAAAATCTG |
| | | | | TACGGGCGTGAGTGCATATATATATATATTGCGGTTTGGAAAAAAA |
| | | | | AAAAGAAAAACCTTTAAACAAAAAAATATGATTGAGCACGTTGACA |
| | | | | TAAAAATGTAATGTGTACCGACCACGGTCAATATGTCCTTACCACCC |
| | | | | GCCGCCTCCACCTTTAAGAGAGAGAGAGAGAGAGAGAGAGAATCA |
| | | | | TTATCAACATCATATAATAACACCGGAAAAGCAAAGCAACAAAAAT |
| | | | | GAGTGTCACAGCCACATTGCAGTCTGTCTCTACTCCGGCGTCACCAC |
| | | | | TAAGCCAGGGCTGCAGATTGGAACTACCACCTAAACTGCTTTCTTTC |
| | | | | ACTAAATCAAGTGTTGGTCGAAGGCTTGCTTTTAAGCTGGTTAATGC |
| | | | | TATCTCCGACTCCGGTAATGGAGCTCCGGCTTCACTTCTTTCTACTTC |
| | | | | TTCCCTTAATGGAGTTACTGCCCCGCCACTTTCTTCCAAGTATAACTC |
| | | | | TTCTCTTTTCTTTAATTCCTTCTTAAATTACTAGTAACTATAATAATAT |
| | | | | TTGTTTTTATAGAACCTTTCATGTTTTTCTTTACCCAAGCAGCTTATA |
| | | | | ATTAATAACTTTTTGCAAAAGCTTAATTAAAAAGCATTTGTCATATA |
| | | | | ATAGCTAGAAGGAAGGCAGGATTAGCTTTCAAGCAGTCTTCAAGCA |
| | | | | TGACATGAATTCAAGACAGTCTGCTTCTGAGAATCTGATTCTACTTT |
| | | | | CATGCCACCTTTCACTTCCCCGTGGCAATGCCTTTGATTCTGGCTTTA |
| | | | | GATTAGGGTCTTGCTTACAATGTTGTTATTTAAATTGTACATTTTTGT |
| | | | | CAGTAATTTTAGTGTAACCGAGGTATTTAGCTCAACTTTTTTTGGGG |
| | | | | TTAATTGTTTCAACTACTCGTAACCGAATTTTGATTGTTGTTTAAGAT |
| | | | | TCTCTTGCAACAAAGTTCTCAACTTTAAGCCTATAGTATGAACATATT |
| | | | | TCCTTGGAGTTATCCCAGAGAGATATTCGAGATACCTAGGCACATA |
| | | | | CGGGTCAATAAGTTATTTAGGGAAAATGATTACAGTATTTGGAGGA |
| | | | | ATACCCGTTGCACCGAAACTCTTCCAACATGTGCCAGTACATTTGTA |
| | | | | AAGTTACTGCAACTTAGTAAACTCTATGGGTAATACAACCATGTGTT |
| | | | | TACAGGTAGGAGTATCTTTTTGATAAAATTTATTTGGTTTATGTATT |
| | | | | GACCAGGGTGAAGCGCCACACTATCTCGGTGTTCGTAGGAGATGA |
| | | | | AAGTGGTATTATAAACCGAATCGCAGGAGTTTTCGCTAGAAGAGGT |
| | | | | TATAATATCGAGTCACTTGCTGTTGGTTTAAACAAGGACAAGGCTTT |
| | | | | GTTTACTATAGTTGTTTCTGGAACTGAAAAGGTCTTGCAACAAGTGG |
| | | | | TGGAGCAGCTCAACAAGCTTGTGAACGTCATAAAGGTTTGTATGAC |
| | | | | CATCAAATGTGAATCTTGGGTGCTCTTTGAACTTTATACATGATTAA |
| | | | | CGAGTTTGATGTATCAGGTGGAAGACCTTTCCAAGGAACCACAGGT |
| | | | | AGAACGGGAATTGATGCTTGTGAAGCTTAATGTAGATCCAACCACA |
| | | | | CGAGCTGAGGTTTTAGTGTTTTGCATACTTCTATGATATTCAATTATA |
| | | | | GAAAATGGCAGAGAGAATATTTTATTTGAGTCTGATACAGGGTTAC |
| | | | | TTTTACCCTTTAGATTATGTGGCTAGTAGACGTCTTTAGAGCCAACG |
| | | | | TTGTGGATGCATCAGAAAGCTCGTTGACTATAGAGGTACGTAGCTT |
| | | | | CTTATTCATTCAATCTGAACAGTTGCATACTCCCACAGTCTCACTGTT |
| | | | | TTGTTATTAGAACATGCGTCAACAAAAAAAAAAGTTTGTAATTTTGA |
| | | | | TAATATTGCTAGTAATTATGTAATGCTAATTTTATCATCTGGTCAATT |
| | | | | TAGGTTACCGGGGATCCTGGGAAGATTGTTGCTGTTCAGAGAAATT |
| | | | | TAGCCAAGTTCGGAATCCAAGAGCTCACTAGAACAGGAAAGGTAT |
| | | | | GCAAGCACAAGCATAAGTTTTCTAAATTTCTGTTATGTGTTGCATAT |
| | | | | ATTTCATAGAAATTTAAATTTTCTACCAGAATGATAGTTGGTCGCAA |
| | | | | AGACCATAAATGTCCATTGAATCTCCTGACTGAATTTGTTGTAACTTT |
| | | | | TAGATCGCTCTAAGACGGGAAAAACTGGGTGAAACTGCTCCATTTT |
| | | | | GGAACTTCTCTGCAGCATCTTACCCAGATCTAGAATCCGCAACTCCA |
| | | | | ATTGCTACTTCTTCAAGTGTTACTTGCACTGTTGATGAGGATTCGAC |
| | | | | TGCAATGTCAGGGGTAACCTGTCTAATCTTTAAAGGCTTTCCTTGCA |
| | | | | ATGTTTTGCTCCCTAATTGCATATCACCACCTTATAACTCTATTCTGTT |
| | | | | TAGCATTTAGGAATTGTGACAGAGATAGAAAGGCAATGTATAAAAT |
| | | | | ATCAATGATACAATTGAATGATAAAATGATAAAGACTTTTACTTTGA |
| | | | | TTTCTGCAAGACACAATATATGTAAGTTACTAAATCTGCAGTTTCAC |
| | | | | ACAACAATTAATTCTTTAGTTGGCACACTTTCACTGTGTCTGGTACTG |
| | | | | CTTAGACCAAATTTCTACTTATTTAACACTTTAATCTGTTAAGTGGTT |
| | | | | AACAAGATTTGAACAACCTAGTTGTTAAATTGATTTTTGATTTCATTT |
| | | | | GAGTTATTAATAATCTTGTTTGAATGAAGGGTGATGTGTATCCTGTG |
| | | | | GAGTACAACAATAGCTGCATAATGGATCAAGTTCTTGACGCCAATT |
| | | | | GGGGGGTAGTCTATGATGAAGATGTAAGATATCAATTTATCATCTTT |
| | | | | CATTTGTTCCTTATCGCTATATTTATACTTGATCCCTTTATTGATGAA |
| | | | | AGACAATTATATCTTAGATGGATCATAACTTATATGTTTCGCCAAAC |
| | | | | ACACTTCTAGTTTCTAAGATAGCGAACAGTTCAGCTTAATTAGTTAA |
| | | | | TATTCTTCAAGCTAAAGATGTATGTTGTTATTCTCATTCTAACACCCA |
| | | | | CAGGTTGTTACTATTTAGAAAAGGGGAAATAGTTATGGCATATAAT |
| | | | | CGTGTTTACTGAGGCTTGTGACTACTCATAGATTTCACTTTTGACAC |
| | | | | CCAAATTAATGTTGCAGTCAACTGGTCGGCAGTCTCATACTTTAAAC |
| | | | | ATTCTGGTCAATGATACTCCTGGAGTTCTCAACGTGGTCAGTGGAGT |
| | | | | TATATCCAGAAGGGGTTACAACATTCAGGTTCTTCTTCTTTTTTGCTA |
| | | | | ATTCTGAAGATACAATTGTTTTAAAGTAAGATGTAGTTTGCAAATAA |
| | | | | TACTAATAATCGTCTTTGGAACTGGTGTCAACAGAGTCTGGCCGTA |
| | | | | GGTCCAGCAGAAATGGAGGGTTTATCTCGCATTACGACTGTGATTG |
| | | | | CTGGTACAGATGAATCTATTTACAAGTTGGTTCAGCAGTTCCGCAAA |
| | | | | TTGGTGGATGTTCATGAGGTGAGATTTATTTCTTTAAAGTTAAACCT |
| | | | | CTTGAAATTCTCATAAAATCGACCACTATAATTTTCGCTGTATCTTAC |
| | | | | TTCAGGCATGGAAAACTTATAACTTAATGAAAGAAAATATTGTTTAT |
| | | | | ATTTAGGTCAAAGATATCACCCATCTACCATTTGCAGAACGTGAGCT |
| | | | | AATGTTAATTAAAGTTGCTGCTAGCTCTGCTGTTCGAAGGGATGTCC |
| | | | | TAGACATCGCCACCATCTTTCGAGCCAAGCCTGTTGATGTTTCTGAT |
| | | | | CACACTCTCACACTAGAAGTAAATTTTCTACGTATTGATTTTGAGGT |
| | | | | GTTTCTATGTCAAATATTCTTGTTTCTGATATCTTCTGTTATTCATATT |
| | | | | TCAGCTCACAGGAGACTTCAACAAGTTGTTTGCATTGCAAAGATTGT |
| | | | | TAGAGTCCTATGGAATTTGTGAGGTTCGTTCAAACTTTTGACCTCGA |
| | | | | ACTCCAATACTGGGTTTCACTTTTCAATCTTCTGCACAACTTTATTAT |
| | | | | GGCCTGCTCCTATCTGGTTAGGGACTTGGGGTTCTCCCCAGCAATTC |
| | | | | ATTCTCTCAAACCCCTAGTCTCGACATTGTATCATGCCTCACTCTGAT |
| | | | | ATATCTTCTATTTTGGAGCTGTATTACTATCCAAATTGTGTTTATTAC |
| | | | | ACGAAATATTATTACTAGTAAAAACAATAGTCATTAAGGTATAACTG |
| | | | | TGCATGGTCTTTGATGCTGTTTTCTCTGCATTGCATCATTTCCCTAAT |
| | | | | GGTGCCTAATTGCATTTTAATATGCAGGTCGCAAGGACTGGTCGAG |
| | | | | TGGCATTGGTGCGAGAGTCAGGGGTCGATTCTACATATCTACGTGG |
| | | | | GTACTCCCTTCCGTTATAATAACACCGGAAGTTATCCCTCAATTGTAT |
| | | | | GTTGCATTATTGTAGCTGCAAGTGAACACAACTGTTCAGTGTCTTTT |
| | | | | GCACTGATGGCTGCGGATGTGAGAGTATGACTTCTTGCTAGAAAAA |
| | | | | GAAAAAAGGAATGTTTAGTAGATCAATTTATGTTGTATTATGGTATG |
| | | | | CCGTGTGATGTCAAAACTGTAACAGCTTTCATTCATAGAAGTTTCAT |
| | | | | ACTTAAAATGTGATGTTAAGCAAATTTATAAGTAATAATGTTTGTAC |
| | | | | TTCTATACTTCTATATAAAAGTTATAATCCCATATTGAAAGTTAAAAA |
| | | | | AAATAAGACATACAAATTGACTAAATTACTTTTAATGAATTAAAATC |
| | | | | ACCACCCTTTAATATTAAATTACACCATCAGCTCCTTATATTATAAAA |
| | | | | GATTTATATTACCTTCTTTAAATCAAACACGTTTACATTAATTACCTA |
| | | | | CACCATTTGACGTCGTGACCACCATCACCATCCGTCGCCGCCATCAT |
| | | | | ACTACCACATTGCGCGATAATGTGCTAGTTATATACCTAGAGATTAT |
| | | | | TGGTGGAATTGGTTGAAATATACGAGTCTTTATTTTGTCAACGTTAT |
| | | | | ACACTCGAGGGGTAGAACTGAACAAATTATGTATGTACTTGGTGGA |
| | | | | ACGGTGGGACAAAATGAGGAGAAAAGCTATATAATACTTGTCCCTG |
| | | | | ACTTCTTGAGCCATTTTAATGAAGCAAATGTCCTCCAAAAAACCTTTT |
| | | | | TTCTTCGGGTCGAGCGTTAGCATCATTATAACATTTTAGACTAATTTC |
| | | | | TAAGCATTCCATTATGATCCTCAGTATACCAGACTTTATACAGCTTTG |
| | | | | AGAAATAAAACACTTGATATTTCTTGCACAAATATTTGCCATTATGA |
| | | | | TCTCAGTATACCAGACTTTATAAAGATTATATATACAAAACCCAAGT |
| | | | | AACCCAAGACTTAACAACATTCGACTTCTAACCTTAATAAGTAATAC |
| | | | | ATCACATACCCATCCCCATCCCATCCACCCAAAAGCCGAGAAAAAAA |
| | | | | AAAGTTATGAACACATAATTAACACTTATACTCGAGGCAAAAAAAA |
| | | | | CTCTCAATAACCATGCCCTCTTCTGGAGACCTGGACAACGTATAAGA |
| | | | | ATCATTCTCTTGTCAAAATGAATATATATCGAGATCAGGAAGATGAT |
| | | | | CACCTCATCTTGTGAATAACTAAGCCAGATAACTCTTAAGGAGTATG |
| | | | | CAGATCGCGTAAATGGTCAACAACATCACGTGAACCAGTCAACCAC |
| | | | | TTGATATCAGAACGGTGAAGTACATTTGCATGGACACGGGCTGAAT |
| | | | | TATCAGGTTTCTGCTCCATAGGCATTGACCTCTCTGAAAAGCTGTGG |
| | | | | GTATGGCGCACACGAACTCTGTGACATTTTGTTTCTGTTGTACAGCT |
| | | | | GCCCCTCACTTCAACCATCAACATAACTTCCTTATCACGAACGTCTTC |
| | | | | GCACCAGCAGTTTTGCGGAGCACCATCTACATATTCCTCTAGGGTTT |
| | | | | CATATCCTTGGTGATGAACTGATATCTCTACAATATGATTTGGCTCT |
| | | | | ATTATACCCACTGTTGGCTTGACCTGGTTCAAAGCAATAGACAATTG |
| | | | | TTAGCTACCTTCACCGTGAGTGCATCACAACAAATTTTTTAGTCCAAT |
| | | | | GTTCAATGCATGTGATAAATTCACCTCAAGCCATCGTGGAAAGCCAT |
| | | | | AAGAACCTCTTGGACGGTGACTGGAAGCCTGTTTTCCATCTTTAATA |
| | | | | GTACACTGGCCTTCACATATAACTTTAAACAGAGCTTCATCTTCCCC |
| | | | | GCATTTATTGGTAATCCTCAAGACTGATGAATCCCTGTCATGAAGAA |
| | | | | TTATATTGTTCGTACTGGGAATAGCTTCTGGAACATTGCACTGTTCT |
| | | | | TCAAGAATACTCCTAATTTTTTTATTAGATTTGATAACTTCTCCAAAT |
| | | | | ATTTTTCTTCTTCTTGACTCATCTACTCGAGCCAGTTCCACGTTAAAG |
| | | | | ATGCAACGCACAGGCTTATGATCACTATCTGTCACAT |
| 1747 | Conyza | gDNA | 5498 | TTAGATTAGGGTCTTGCTTACAATGTTGTTATTTAAATTGTACATTTT |
| | canadensis | Contig | | TGTCAGTAATTTTAGTGTAACCGAGGTATTTAGCTCAACTTTTTTTG |
| | | | | GGGTTAATTGTTTCAACTACTCGTAACCGAATTTTGATTGTTGTTTAA |
| | | | | GATTCTCTTGCAACAAAGTTCTCAACTTTAAGCCTATAGTATGAACA |
| | | | | TATTTCCTTGGAGTTATCCCAGAGAGATATTCGAGATACCTAGGCAC |
| | | | | ATACGGGTCAATAAGTTATTTAGGGAAAATGATTACAGTATTTGGA |
| | | | | GGAATACCCGTTGCACCGAAACTCTTCCAACATGTGCCAGTACATTT |
| | | | | GTAAAGTTACTGCAACTTAGTAAACTCTATGGGTAATACAACCATGT |
| | | | | GTTTACAGGTAGGAGTATCTTTTTGATAAAATTTATTTGGTTTATGT |
| | | | | ATTGACCAGGGTGAAGCGCCACACTATCTCGGTGTTCGTAGGAGAT |
| | | | | GAAAGTGGTATTATAAACCGAATCGCAGGAGTTTTCGCTAGAAGAG |
| | | | | GTTATAATATCGAGTCACTTGCTGTTGGTTTAAACAAGGACAAGGCT |
| | | | | TTGTTTACTATAGTTGTTTCTGGAACTGAAAAGGTCTTGCAACAAGT |
| | | | | GGTGGAGCAGCTCAACAAGCTTGTGAACGTCATAAAGGTTTGTATG |
| | | | | ACCATCAAATGTGAATCTTGGGTGCTCTTTGAACTTTATACATGATT |
| | | | | AACGTGTTTGATGTATCAGGTGGAAGACCTTTCCAAGGAACCACAG |
| | | | | GTAGAACGGGAATTGATGCTTGTGAAGCTTAATGTAGATCCAACCA |
| | | | | CACGAGCTGAGGTTTTAGTGTTTTGCATACTTCTATGATATTCAATTA |
| | | | | TAGAAAATGGCAGAGAGAATATTTTATTTGAGTCTGATACAGGGTT |
| | | | | ACTTTTACCCTTTAGATTATGTGGCTAGTAGACGTCTTTAGAGCCAA |
| | | | | CGTTGTGGATGCATCAGAAAGCTCGTTGACTATAGAGGTACGTAGC |
| | | | | TTCTTATTCATTCAATCTGAACAGTTGCATACTCCCACAGTCTCACTG |
| | | | | TTTTGTTATTAGAACATGCGTCAACAAAAAAAAAAGTTTGTAATTTT |
| | | | | GATAATATTGCTAGTAATTATGTAATGCTAATTTTATCATCTGGTCAA |
| | | | | TTTAGGTTACCGGGGATCCTGGGAAGATTGTTGCTGTTCAGAGAAA |
| | | | | TTTAGCCAAGTTCGGAATCCAAGAGCTCACTAGAACAGGAAAGGTA |
| | | | | TGCAAGCACAAGCATAAGTTTTCTAAATTTCTGTTATGTGTTGCATA |
| | | | | TATTTCATAGAAATTTAAATTTTCTACCAGAATGATAGTTGGTCGCA |
| | | | | AAGACCATAAATGTCCATTGAATCTCCTGACTGAATTTGTTGTAACT |
| | | | | TTTAGATCGCCCTAAGACGGGAAAAACTGGGTGAAACTGCTCCATT |
| | | | | TTGGAACTTCTCTGCAGCATCTTACCCAGATCTAGAATCCGCAACTC |
| | | | | CAATTGCTACTTCTTCAAGTGTTACTTGCACTGTTGATGAGGATTCG |
| | | | | ACTGCAATGTCAGGGGTAAGCTGTCTAATCTTTAAAGGCTTTCCTTG |
| | | | | CAATGTTTTGCTCCCTAATTGCATATCACCACCTTATAACTCTATTCT |
| | | | | GTTTAGCATTTAGGAATTGTGACAGAGATAGAAAGGCAATGTATAA |
| | | | | AATATCAATGATACAATTGAATGATAAAATGATAAAGACTTTTACTT |
| | | | | TGATTTCTGCAAGACACAATATATGTAAGTTACTAAATCTGCAGTTT |
| | | | | CACACAACAATTAATTCTTTAGTTGGCACACTTTCACTGTGTCTGGTA |
| | | | | CTGCTTAGACCAAATTTCTACTTATTTAACACTTTAATCTGTTAAGTG |
| | | | | GTTAACAAGATTTGAACAACCTAGTTGTTAAATTGATTTTTGATTTCA |
| | | | | TTTGAGTTATTAATAATCTTGTTTGAATGAAGGGTGATGTGTATCCT |
| | | | | GTGGAGTACAACAATAGCTGCATAATGGATCAAGTTCTTGACGCCA |
| | | | | ATTGGGGGGTAGTCTATGATGAAGATGTAAGATATCAATTTATCAT |
| | | | | CTTTCATTTGTTCCTTATCGCTATATTTATACTTGATCCCTTTATTGAT |
| | | | | GAAAGACAATTATATCTTAGATGGATCATAACTTATATGTTTCGCCA |
| | | | | AACACACTTCTAGTTTCTAAGATAGCGAACAGTTCAGCTTAATTAGT |
| | | | | TAATATTCTTCAAGCTAAAGATGTATGTTGTTATTCTCATTCTAACAC |
| | | | | CCACAGGTTGTTACTATTTAGAAAAGGGGAAATAGTTATGGCATAT |
| | | | | AATCGTGTTTACTGAGGCTTGTGACTACTCATAGATTTCACTTTTGA |
| | | | | CACCCAAATTAATGTTGCAGTCAACTGGTCGGCAGTCTCATACTTTA |
| | | | | AACATTCTGGTCAATGATACTCCTGGAGTTCTCAACGTGGTCAGTGG |
| | | | | AGTTATATCCAGAAGGGGTTACAACATTCAGGTTCTTCTTCTTTTTTG |
| | | | | CTAATTCTGAAGATACAATTGTTTTAAAGTAAGATGTAGTTTGCAAA |
| | | | | TAATACTAATAATCGTCTTTGGAACTGGTGTCAACAGAGTCTGGCCG |
| | | | | TAGGTCCAGCAGAAATGGAGGGTTTATCTCGCATTACGACTGTGAT |
| | | | | TGCTGGTACAGATGAATCTATTTACAAGTTGGTTCAGCAGTTCCGCA |
| | | | | AATTGGTGGATGTTCATGAGGTGAGATTTATTTCTTTAAAGTTAAAC |
| | | | | CTCTTGAAATTCTCATAAAATCGACCACTATAATTTTCGCTGTATCTT |
| | | | | ACTTCAGGCATGGAAAACTTATAACTTAATGAAAGAAAATATTGTTT |
| | | | | ATATTTAGGTCAAAGATATCACCCATCTACCATTTGCAGAACGTGAG |
| | | | | CTAATGTTAATTAAAGTTGCTGCTAGCTCTGCTGTTCGAAGGGATGT |
| | | | | CCTAGACATCGCCACCATCTTTCGAGCCAAGCCTGTTGATGTTTCTG |
| | | | | ATCACACTCTCACACTAGAAGTAAATTTTCTACGTATTGATTTTGAG |
| | | | | GTGTTTCTATGTCAAATATTCTTGTTTCTGATATCTTCTGTTATTCATA |
| | | | | TTTCAGCTCACAGGAGACTTCAACAAGTTGTTTGCATTGCAAAGATT |
| | | | | GTTAGAGTCCTATGGAATTTGTGAGGTTTGTTCAAACTTTTGACCTC |
| | | | | GAACTCCAATACTGGGTTTCACTTTTCAATCTTCTGCACAACTTTATT |
| | | | | ATGGCCTGCTCCTATCTGGTTAGGGACTTGGGGTTCTCCCCAGCAAT |
| | | | | TCATTCTCTCAAACCCCTAGTCTCGACATTGTATCATGCCTCACTCTG |
| | | | | ATATATCTTCTATTTTGGAGCTGTATTACTATCCAAATTGTGTTTATT |
| | | | | ACACGAAATATTATTACTAGTAAAAACAATAGTCATTAAGGTATAAC |
| | | | | TGTGCATGGTCTTTGATGCTGTTTTCTCTGCATTGCATCATTTCCCTA |
| | | | | ATGGTGCCTAATTGCATTTTAATATGCAGGTCGCAAGGACTGGTCG |
| | | | | AGTGGCATTGGTGCGAGAGTCAGGGGTCGATTCTACATATCTACGT |
| | | | | GGGTACTCCCTTCCGTTATAATAACACCGGAAGTTATCCCTCAATTG |
| | | | | TATGTTGCATTATTGTAGCTGCAAGTGAACACAACTGTTCAGTGTCT |
| | | | | TTTGCACTGATGGCTGCGGATGTGAGAGTATGACTTCTTGCTAGAA |
| | | | | AAAGAAAAAAGGAATGTTTAGTAGATCAATTTATGTTGTATTATGG |
| | | | | TATGCCGTGTGATGTCAAAACTGTAACAGCTTTCATTCATAGAAGTT |
| | | | | TCATACTTAAAATGTGATGTTAAGCAAATTTATAAGTAATAATGTTT |
| | | | | GTACTTCTATACTTCTATATAAAAGTTATAATCCCATATTGAAAGTTA |
| | | | | AAAAAAATAAGACATACAAATTGACTAAATTACTTTTAATGAATTAA |
| | | | | AATCACCACCCTTTAATATTAAATTACACCATCAGCTCCTTATATTAT |
| | | | | AAAAGATTTATATTACCTTCTTTAAATCAAACACGTTTACATCAATTA |
| | | | | CCTACACCATTTGACGTCGTGAACACCATCACCATCCGTCGCCGCCA |
| | | | | TCATACTACCACATTGCGCGATAATGTGCTAGTTATATACCTAGAGA |
| | | | | TTATTGGTGGAATTGGTTGAAATATACGAGTCTTTATTTTGTCAACG |
| | | | | TTATACACTCGAGGGGTAGAACTGAACAAATTATGTATGTACTTGG |
| | | | | TGGAACGGTGGGACAAAATGAGGAGAAAAGCTATATAATACTTGT |
| | | | | CCCTGACTTCTTGAGCCATTTTAATGAAGCAAATGTCCTCCAAAAAA |
| | | | | CCTTTTTTCTTCGGGTCGAGCGTTAGCATCATTATAACATTTTAGACT |
| | | | | AATTTCTAAGCATTCCATTATGATCCTCAGTATACCAGACTTTATACA |
| | | | | GCTTTGAGAAATAAAACACTTGATATTTCTTGCACAAATATTTGCCA |
| | | | | TTATGATCTCAGTATACCAGACTTTATAAAGATTATATATACAAAAC |
| | | | | CCAAGTAACCCAAGACTTAACAACATTCGACTTCTAACCTTAATAAG |
| | | | | TAATACATCACATACCCATCCCCATCCCATCCACCCAAAAGCCGAGA |
| | | | | AAAAAAAAAAGTTATGAACACATAATTAACACTTATACTCGAGGCA |
| | | | | AAAAAAACTCTCAATAACCATGCCCTCTTCTGGAGACCTGGACAAC |
| | | | | GTATAAGAATCATTCTCTTGTCAAAATGAATATATATCGAGATCAGG |
| | | | | AAGATGATCACCTCATCTTGTGAATAACTAAGCCAGATAACTCTTAA |
| | | | | GGAGTATGCAGATCGCGTAAATGGTCAACAACATCACGTGAACCAG |
| | | | | TCAACCACTTGATATCAGAACGGTGAAGTACATTTGCATGGACACG |
| | | | | GGCTGAATTATCAGGTTTCTGCTCCATAGGCATTGACCTCTCTGAAA |
| | | | | AGCTGTGGGTATGGCGCACACGAACTCTGTGACATTTTGTTTCTGTT |
| | | | | GTACAGCTGCCCCTCACTTCAACCATCAACATAACTTCCTTATCACGA |
| | | | | ACGTCTTCGCACCAGCAGTTTTGCGGAGCACCATCTACATATTCCTC |
| | | | | TAGGGTTTCATATCCTTGGTGATGAACTGATATCTCTACAATATGAT |
| | | | | TTGGCTCTATTATACCCACTGTTGGCTTGACCTGGTTCAAAGCAATA |
| | | | | GACAATTGTTAGCTACCTTCACCGTGAGTGCATCACAACAAATTTTT |
| | | | | TAGTCCAATGTTCAATGCATGTGATAAATTCACCTCAAGCCATCGTG |
| | | | | GAAAGCCATAAGAACCTCTTGGACGGTGACTGGAAGCCTGTTTTCC |
| | | | | ATCTTTAATAGTACACTGGCCTTCACATATAACTTTAAACAGAGCTTC |
| | | | | ATCTTCCCCGCATTTATTGGTAATCCTCAAGACTGATGAATCCCTGTC |
| | | | | ATGAAGAATTATATTGTTCGTACTGGGAATAGCTTCTGGAACATTGC |
| | | | | ACTGTTCTTCAAGAATACTCCTAATTTTTTTATTAGATTTGATAACTTC |
| | | | | TCCAAATATTTTTCTTCTTCTTGACTCATCTACTCGAGCCAGTTCCAC |
| | | | | GTTAAAGATGCAACGCACAGGCTTATGATCACTATCTGTCACA |
| 1748 | Conyza | gDNA | 1105 | CTCTCTCTCTACTTTCTATTGTTTTCTCTTGAGAAAAAGTTGGAACCT |
| | canadensis | Contig | | TTTTCTCAGAAAAAGTGAGAAAACTTGCGTTGGGAAGGGCCTTACC |
| | | | | AACTTTATATACACATAGAATTCAGTTGTGTTGCATACTTGCATGAG |
| | | | | GCATAAGATAATAAGGGAGCTCAAATTCTGACATTAATGGAATTTC |
| | | | | ACCCTAACAATAATATCTCCATTCTCTTCAAATCCGATCCTTAGTTTT |
| | | | | CTACAGCCACATCATCGCCACATCATACACATATATTATACATACAC |
| | | | | AAATTCAAAAATTCTCTCGTCTTCCCGAGGATACCATCCCAGAACTT |
| | | | | GGGTAGAACGACTTCGTAACTATAAAGTTTTGAGAAAGCAGCTTCG |
| | | | | GAACATAGAAGAACGTGCTCCAGAACGTGTGGTGGCAATAAAGAC |
| | | | | ACGAGAACTTGGGTTCTAGAACGCCTTAATTATTATCTTAATCATTA |
| | | | | TCATTTAGTATCATACTAGATTTTAGACCCGTGTCAAATACCCGGGT |
| | | | | TTTACAACATTATTGATATAAGAATTAGTATATGGAAAAAAATTATA |
| | | | | CGTTAAATGTAAAAATACACTTAAAAAAACTGAGATATTTAAATGTT |
| | | | | AATACTGAATGCTAAATCATATAATTAAGGCTGGATGGTGTAACAA |
| | | | | AGTTGTCTTTGATACACACTTTTATAGAAAAGCAATATCTTAACTTTG |
| | | | | ATTACACTATTGACTGAGTTTTCATTGATGTCTTGCAAAAAAGTAAT |
| | | | | TTTATTTTTTGTCAGACATGATTGTTAAGAACAATAAAAGTAGCGGC |
| | | | | TGTTTCGCGAGATTTTTAAAGATTTCGTTTATTGTTATTTTTTTTTATG |
| | | | | TTCAATTTATATAAAAAAAATCTATATCCTAATAAATATAGTTTGATT |
| | | | | CCAATTAATAATAACAACAATGATTATCCTAATTTTAGTCTGATTAG |
| | | | | GAATAGTAATAATATTTGATTATAGTTAATAATATAAAATAAAATAT |
| | | | | TGATATAAATATGTAAACTTACTAACTTTTAGCTATATTTTGTTATTA |
| | | | | AATTAGATATTATGCAATAAAATATTAATTATTTTTAAATTGGATTAA |
| | | | | ATGTGTAAATTGGTGATGGAAA |
| 1749 | Conyza | gDNA | 246 | AAGGAAAGGCGAAATGAATTGGGTGGGGTGGAAGAGGAGGTGGA |
| | canadensis | Contig | | CTGTCATGTCACCAGAAAGGCCATATCACACGCTGACGTGGCAAAA |
| | | | | TATTGCTATATTGAGTAATTTTTGAAAGGTCATTTCTAATATCGACAA |
| | | | | GTTTTAAAGGTGACTGCTAGTTGCGTGAATTGGACCAAAGTTGCTTT |
| | | | | CTATTTTGTGCGGATTTCCCTTAAAAGAAAGGAAAGTGATGGAAAG |
| | | | | GAAAAGTGAGGGGTT |
| 1750 | Euphorbia | cDNA | 598 | GGAGCCTTATGATGAATTCTCTGTGCATCAAGTTCTCGATGCTCATT |
| | heterophylla | Contig | | GGGGAGTTCTCTATGATGAAGATTCTAGTGGACTTCGATCACATAC |
| | | | | GTTATCCATACTTGTGAATGATTCTCCTGGTGTTCTCAACACGATTAC |
| | | | | CGGGGTTATGTCTCGGCGAGGTTATAACATCCAGAGTCTTGCTGTG |
| | | | | GGTCGAGCTGAGAGAGAGGGACTTTCTCGCATTACAACTGTTATCC |
| | | | | TTGGAAATGATGATACAGTTAGAAAGTTGGTTCTGCAACTTCACAA |
| | | | | GTTGATTGATATACATGAGGTACAAGATATTACACATTTGCCATTTG |
| | | | | CAGAGCGAGAGTTAATGCTGATAAAGGTGGCAGTCAATACTGCAG |
| | | | | CCAGGAGAGATGTCCTAGATATTGCTAGCATATTTCGGGCCAAAGC |
| | | | | TGTTGATGTGTCTGACCACACAATTACCCTAGAGCTTACTGGTGATT |
| | | | | TAACAAAGATGGCGGCCCTGCAGAAACTATTAGAGCCATATGGGAT |
| | | | | TTGTGAGGCAGCACGGACTGGAAGGGTGGCATTGGTGAGGGAATC |
| | | | | AGGAGTGGATTCCACTTATCTCCGTGGGTACCATCTCCCTTTG |
| 1751 | Euphorbia | cDNA | 598 | CCCCCCTTACCTCGCTCCAAGGTGCAGTTGCACACGATATCAGTCTT |
| | heterophylla | Contig | | TGTGGGTGATGAGAGCGGGATGATAAATAGAATAGCTGGCGTTTTT |
| | | | | GCTAGAAGGGGTTACAACATTGAGTCCCTTGCTGTTGGTTTGAACA |
| | | | | AGGACAAGGCTCTTTTTACTATTGTTGTTTCCGGAACTGACAAGGTT |
| | | | | TTGCAACAAGTTGTCGAGCAGCTCAACAAGCTTGTCAATGTCATCAA |
| | | | | GGTGGAAGATATCTCTAAGGAACCACATGTGGAACGTGAACTGAT |
| | | | | GCTTATAAAACTTAATGTGGATCTAGAAACTCGTCCCGAGATTATGT |
| | | | | GGCTGTTGGATGTCTTCAGAGCTAAAGTTGTTGATACCTCAGAGCA |
| | | | | TACAATGACTATTGAGGTTACCGGAGACCCTGGTAAAATGGTTGCT |
| | | | | GTTGTAAGGAACCTAAGCAAGTTCAGAATCAAAGAGATTTCTAGAA |
| | | | | CTGGAAAGATTGCTCTAAGACGGGAAAGGATGGGCGAGACAGCAC |
| | | | | CGTTCTGGAGGTTTTCTGCAGCTTCCTATCCGGACCTTGAAAGTATG |
| | | | | CAACCTGGTGCTGTTGAAAATGTGCAACCTGCTGGTGCCTCAC |
| 1752 | Euphorbia | cDNA | 595 | GATAGCTTTACCGTAACTCAAGTTCTCGATGCTCATTGGGGTGTCCT |
| | heterophylla | Contig | | CAATGAGGAAGATACAACTGGACTTCGATCCCACACTCTATCTATG |
| | | | | GTAGTAAATGACTCTCCTGGAGTTCTCAACATCGTGACAGGGGTAT |
| | | | | TTGCTCGAAGGGGCTATAACATTCAGAGTTTGGCAGTTGGGCATTC |
| | | | | AGAAATTGAGGGGCGCTCACGAATTACAACTGTGGTTCCTGGTACT |
| | | | | GATGAATCAATTAGCAAGTTGGTGCAGCAACTTTACAAGCTAATAG |
| | | | | AGCTCCACGAGGTTCGGGATCTTACCCACTTGCCATTTGCTGAACGA |
| | | | | GAGTTGATGCTGATAAAAATTGCGGTGAATGCTGCTGCACGGCGTG |
| | | | | ATGTCCTCGACATTGCCAGCATATTCAGAGCAAGAGCCATTGATGT |
| | | | | ATCGGACCACACAATAACTCTCGAGCTTACCGGAGATCTCGACAAG |
| | | | | ATGGTCGCATTGCAAAGGTTGTTGGAACCCTATGGCATTTGCGAGG |
| | | | | TGGCAAGAACAGGGCGAATAGCATTGGTTCGAGAGTCTGGTGTGG |
| | | | | ATTCTAATTATCTCCGTGGATACTCGTTTCCTATATAATAAA |
| 1753 | Euphorbia | gDNA | 5587 | ATTGTTGAGTTTAAAAATGCAAAATTGATATAATATTTTTTATTATGA |
| | heterophylla | Contig | | AATAGATGTAGAGAGTACTCATAATTTAGTATTTTTTTTAAAAAAAT |
| | | | | CGAAACCAATTCAACTTAGCCGTGTCTGAAAATATTGAAGGTGGTTT |
| | | | | GTTTGAAATGAGAATTAACAAATAAAAGCCGGCCCAAACCCGTCGC |
| | | | | AATCCGGGCCAGGAACGGTGGTAATAATGACCAATTGCTTAAGAAA |
| | | | | CTCATTAGCCCATATTATTATTTTGATAAAAGGATAAAAGTAATTTA |
| | | | | CACGCGTGATAACGCCAAATTAGCGGGGACATATTAGCAAATAATT |
| | | | | TGAAAATAATTAGGAAATAATTAAAGTTTAATTATGTAAAGACAAC |
| | | | | AGTGTAGTCCGTAGTAGATAGAAGAGGCGAAGGAGAAGGTGGTTA |
| | | | | GTACTGGAGGGCGAAAGGACAGAAGAAGCAGAAAAATATCACATT |
| | | | | TTAATGGTGTTTTGTTGTGGTTTATCTTATTGTACTGTATGAATTGCG |
| | | | | GCGCTTCCGCCATCTGCGTCCCCTCCTTAAGATCTCATTATCTTGTTA |
| | | | | ATCTTACTACCACTACAATCACAATGCCTGCGGCTGCTGCTCTCTCAT |
| | | | | CTTCCCTTGTTGCCGCCGCACCCTCTCACGATTCCTCTCCTTTGACAA |
| | | | | CTCCTTTTTTAAGGAGCAGCAGCAGCAGCTCGGCCCCATTCCCTTTT |
| | | | | CTCCGGCCAAGGACCAATGCTCCTCTTAAGCTCTCCGCTACCCTCAT |
| | | | | CCACCACCCCGGAAACCAACCCGCTCTTCCCCCCTTACCTCGCTCCA |
| | | | | AGTATAAGTCTCACGCTTTTCATGCTTCTTGGATTTTTTTCTAGTATT |
| | | | | AGGCTGCAATTTTATGAACTTAACTGAAGCTCAACTGTAATTGAACT |
| | | | | TATTTCGGTACGAATTTATAACTAAGCATACCTGAACTGAAATGGAT |
| | | | | CAGTCTGATTGTATTCTGAGAGAACAGGGCCTTAGTACTTCCGATTC |
| | | | | CATGTGAAAATGCTTCTGTCTTGTTACTGCTTTTTATTTATGTGTTTC |
| | | | | CTTTTCTTCTATATTTTGAACTTCATGGCCTGATTTTTATATGATGAAT |
| | | | | CTTATGAGTTCGTTATGATTTTTGTTCTAAGTTGCAGGGTGCAGTTG |
| | | | | CACACGATATCAGTCTTTGTGGGTGATGAGAGCGGGATGATAAATA |
| | | | | GAATAGCTGGCGTTTTTGCTAGAAGGGGTTACAACATTGAGTCCCT |
| | | | | TGCTGTTGGTTTGAACAAGGACAAGGCTCTTTTTACTATTGTTGTTT |
| | | | | CCGGAACTGACAAGGTTTTGCAACAAGTTATGGAGCAGTTGCAGAA |
| | | | | ACTTGTTAATGTTTTGAAGGTTGTATTCTTATTTCGAGGGAGTCCCTT |
| | | | | TATCTACTGATTTTAGTTTTATGGTTATAATTGTTGGCACATAGTCCA |
| | | | | ATTGAAGAGAAATGGATACTGGATATATAGAAGAGACAGATTTCTA |
| | | | | TTTGCCTATTTTCATATTATGGCTTTTGTTACATTGTTCGGATTGTAA |
| | | | | TTTCTCCATATGCTGTTGTTTACTATGATATTTAGGTTGAGGATCTAT |
| | | | | CGTCCGAGCCACAGGTAGAACGTGAGCTAATGCTTATTAAACTGAA |
| | | | | GGCAGATGCTAATAACCGTGCTGAGGTAACTGGATTTTCATCGTTG |
| | | | | TTGAGTATGGAAATGCTTTGAGGATTCCAATTTGAATTGCTCCCTTG |
| | | | | TTTTCTTCGGCTACATTTTGTTCTTCGGTGTGATTTTCAGATTATGTG |
| | | | | GTTGGTGGACATCTTCAGAGCAAAAATAGTGGACATCTCTGAGCAT |
| | | | | TCAGTTACAATTGAGGTAAATGATTTGTTAATTGATTCCAATTCTAG |
| | | | | AGATAAAAAGATCCTAGTGGATGACCCCTCTGATACTTTAAAAATTA |
| | | | | AAGATTATTAATAGTTGTTTAATAATATTGACAAATGAATGAACTGG |
| | | | | AAATATTGAACATATTGTTGTTGAAAAGTTGTGCATATTAATTATGA |
| | | | | AGTTAAGTTTGATGCAAACTTGTAGCTAATCATTTTTCTAGTAATTTA |
| | | | | GTTTCAATGATAGTATCAGTGTAATATATGATATGTGGAAATTTTTA |
| | | | | AGGGCTTTAACATCTTAATTTTTCAGCATTGCTATGGAGTAATAGGA |
| | | | | GTTCACTTTTAACTACCAAATATGTTTCTGTTATGATTTTTTAACATT |
| | | | | GTTCCTCATTTCTGATGCTTGTGTTGGTAGCCATGCTTTTCATGATAT |
| | | | | ACTAATTGCTATAGTTGTTTTAGTCTTTCCATTACTTTTACTTGTAAA |
| | | | | ATATATTAAGTGGTATATTAGTTTGTTGAGCTTGGTGAACCTCCATA |
| | | | | TTTTATTTATCTTTTATACCAAGTGCCCAGATTGTTTGTGGTTTATTAT |
| | | | | GGCTATGTTTTTTTTCCCATTGCATGGAAACCTTGGAGGCTTGAACA |
| | | | | GAATTCTAGTTTATGTACGGTAGTTTTGAAAAATGATGTGCGTTGAA |
| | | | | AGTGTAGTTGAAATTTCAAATCAAATGCACCTTTTGAAATTCCTAAA |
| | | | | GGCTAATTTATTGTTATTGGTTTAGGTGACTGGAGATCCAGGGAAG |
| | | | | ATGGTTGCTGTCGAGAGAAACTTAAGCAAGTTTGGAATTAGAGAAA |
| | | | | TTGCAAGAACTGGAAAGGTTGTGTTCAGTTTTCTTTTTATTGTGGCT |
| | | | | GTTTTGTAGGTTTTAGTTTGCCGTTGATTCGTGAGCCTGCTGTTCTG |
| | | | | AATAGATTGCATTGAGAAGGGAAAAAATGGGGGCTTGTGCTCCATT |
| | | | | TTGGCGATTTTCAGCTGCTTCATATCCCGATCTTGGAGAAACAATGT |
| | | | | CGAATAATTCTCTCTTGGAAACTAAAAGTTCAGCAATTGTAGGGGA |
| | | | | AGACACCACATCTTCAGGGGTATGTCAATGTCAAGTTTTTATTTTCTT |
| | | | | TGTTTGGTGTTTTTTCGAGACTATCTGAAAAATTACAACTTTATGAG |
| | | | | CAAATCCAGGGAGATGTGTATCCAGTGGAATCGTCTGATAGCTTTA |
| | | | | CCGTAACTCAAGTTCTCGATGCTCATTGGGGTGTCCTCAATGAGGA |
| | | | | AGATGTAGGTCTGCTTATTTTCAATTCCATCTTTTTTTTTCCGGTCTTA |
| | | | | TAGAGCGCACATTAATTTGTCGAGTATTTCAGACAACTGGACTTCGA |
| | | | | TCCCACACTCTATCTATGGTAGTAAATGACTCTCCTGGAGTTCTCAA |
| | | | | CATCGTGACAGGGGTATTTGCTCGAAGGGGCTATAACATTCAGGTA |
| | | | | TTTCTTATGACTTTCTTCTCTGGTTTGACCTTGTTTCCCTTAATCCTAG |
| | | | | AATGGATTGAAGAACAAATGATTTTCTCGTCTTTGGCGGCAGAGTTT |
| | | | | GGCAGTTGGGCATTCAGAAATTGAGGGGCGCTCACGAATTACAACT |
| | | | | GTGGTTCCTGGTACTGATGAATCAATTAGCAAGTTGGTGCAGCAAC |
| | | | | TTTACAAGCTAATAGAGCTCCACGAGGTCAGAAAACTTCACCAACA |
| | | | | AGTGTCAATTTTTTTGAGTAAATAATGTGTTTTGTGTTTTGATGTAAT |
| | | | | ATTCTATTGTTTTGAAACAAAATGCCTCTATTACCAATCTAATGTCTC |
| | | | | GGAGAAAAAAGAACTTTTTGGTCCCTGTGCTATAAACTTTTTGGCCA |
| | | | | TTTGGTCCATGTGGTATACACTTGACCTTTTTGCTCCCTATTCTATGC |
| | | | | AATATATTACCTTTTTGTCCAGATGTTTTCAATCATTATTCATTACCCA |
| | | | | TTTGGTCCATGTGCAATGCAATTTGTTTATCTTTGTGGACCTTGTTCG |
| | | | | ATAGAGACTTAATTAGTGATATGTCACATATTATGTACTAGAGGGA |
| | | | | CAAAAAAGGTAACAAATTGAATAGCACAGGGACTAAATAGGTAAC |
| | | | | ATGTATATCACCAAAAGGACAAACTGATGATACAACGAGGCTCGAA |
| | | | | AAGGTCTTTTTGCCAATATCTCAGCCTTTGAAGGTTGAATCTTGTATT |
| | | | | TGGTATATATATATATATTTATATAAAGGTCTTCAATCTTGTAAATGG |
| | | | | TGACTTGTCCAGGTTCGGGATCTTACCCACTTGCCATTTGCTGAACG |
| | | | | AGAGTTGATGCTGATAAAAATTGCGGTGAATGCTGCTGCACGGCGT |
| | | | | GATGTCCTCGACATTGCCAGCATATTCAGAGCAAGAGCCATTGATG |
| | | | | TATCGGACCACACAATAACTCTCGAGGTAATTTTCACATTATCTTGTC |
| | | | | ATTGTGTTTAGCCGACAATATGCTCAAACTTATCGCCTTTTTTCATCC |
| | | | | CTGGCTGATGATTTTGCTTTGAATTCTGTTTCCAGCTTACCGGAGAT |
| | | | | CTCGACAAGATGGTCGCATTGCAAAGGTTGTTGGAACCCTATGGCA |
| | | | | TTTGCGAGGTTTGTGGTTAAAACTGTCTCTTCCTTATGCCATAACATC |
| | | | | TTTTTAAAAATTATATCTTTAATTCTTTATCTTTTTATAGGTGGCAAG |
| | | | | AACAGGGCGAATAGCATTGGTTCGAGAGTCTGGTGTGGATTCTAAT |
| | | | | TATCTCCGTGGATACTCGTTTCCTATATAATAAATAGGCCACACTTGT |
| | | | | TATGGTAATCCCCGAGTTTAAACTGGCTTTTGGCTTTTGGCTTTTGTA |
| | | | | TCAGTGTTAACAGTTGAGTGCTCTAGACACATTGTTTTATTTCTTTTT |
| | | | | CATACACAAATAGTATTGAAGTTTTGTTTATAAATTTAAAAATGAAC |
| | | | | TTCACGGGTATTTATTTTTTTAGCAACTAATCGAAATAACTAAAATCC |
| | | | | GGAATCTACATAGAAAATGAGCAAACCAGAGAAGCCTGTCATATAT |
| | | | | ATAGAAGACAATCAACTATCAAGTAAATAACTACAAAATCAGTCTC |
| | | | | GGGCTACGACAACCAATTGTTTCCCGACATTATGGCCAGAAAATAT |
| | | | | CCCTATAAGAGCTTTTGGAGCTTCCTCGAGGCCTTCTGCTACATCTT |
| | | | | CTACATACACAAATTTTCCTTCCTTGATGTTAGGGATAACCAATTGTA |
| | | | | GGAATTTCGGGTAGAGGTGATAGAAATCACCAGCCAAGAATCCTTC |
| | | | | CATTCGAATTCGTTTCCCAATCACATTGCCTAGGTTATATATTCCTTC |
| | | | | GGGATTTTCAAGATTGTATTGAGAAATCATTCCACACACCGCAATTC |
| | | | | TACCTCGAACCCTCATGTTTAGGAGCACTGCGTCCAACATTTTCCCC |
| | | | | CCAACATTCTCAAAGTATATATCTATGCCTTCCGGAAAATACCTGCA |
| | | | | ATTCCAATACAAGACAATCAAAGCTATGAAGCACTTGGTTCAAATTC |
| | | | | AAACCATGGAAACTTGACAGAAAAAATAATAAATTTAAAAAATTTA |
| | | | | CCTTTTGAGTGCTGCATCCAAGTCAGGTTCTTCCTTGTAATTGAAAG |
| | | | | CCTCGTCATACCCGAACTTATTTTTCAGAAGATCAACCTGTAAGAAC |
| | | | | CAAGGAAAACGCAACTCAACAAAAACCTTTAGAAATGTTTACAAGT |
| | | | | TTTCAGGAGGTTGTTGTAAAATGCCAACATAATTCGTCCAACGGCCT |
| | | | | CTTTGAAAGACGATTTTGCCCTTATTTGCGTAATGAAAACCTGAATT |
| | | | | TTCACGGCGCATAAATTACCTTCTCTTTAGAGCCAGCGCTTCCAACA |
| | | | | ACATAGCAACCTGCCAGTTTTGCCAGTTGGCCAACAATCTGACCGAC |
| | | | | CGCACCAGAAGCAGCCGATACGTAGACATATTCTCCT |
| 1754 | Euphorbia | gDNA | 4073 | ATAGGATCTGATAAATTTCTGTTCCGTTGTTCATCAGAAGAATTTTTT |
| | heterophylla | Contig | | TTTTACTATGAGCTCAAATTAAGAAATAAAGCATAAATGTCAGGGTT |
| | | | | TTTATGTAAAAACAACTGAAATATACAACTTATGAAGGAGGGTTTG |
| | | | | TGATCTGTGTGTATGCCAGGTGATTGTCACTTAAGTAGTATAAAAGT |
| | | | | TCATAGCATGCAAAATCAAGACTACTATATGTTCTTTTTAAATGTGT |
| | | | | GTACTATGCTATGGCAAATAGCCTTGTAATGAAGCTTGAATTAGCAC |
| | | | | TAATATAGATTGCTCTAAGACGGGAAAGGATGGGCGAGACAGCGC |
| | | | | CGTTCTGGAGGTTTTCTGCAGCTTCCTATCCGGACCTTGAAAGTATG |
| | | | | CAACCTGGTGCAGTTGAAAATGTGCAACCTGCTGGTGCCTCACCCG |
| | | | | GTAGTCCTGCTACTAATTTTACAGAGAATTCAAATGGTTCCATTAAT |
| | | | | GGCAGAACCATCTCATCTTCAAAGGTTTATTATACTTTAATATATTAT |
| | | | | TTGGTCGATCTAATATCTTATTAGATATGTCCAATATTATTTGTGCAC |
| | | | | TACCATGCCTTGAATATGCCATGTCTTGAATGAAACTTTTAGTACAT |
| | | | | CTACATGGAGATGTGTACAATACTACTGATCTGAATTATTATTAATA |
| | | | | CACTAGATACAGTTAATCATCAGATAGCATCATGTGTTTAGTATCTT |
| | | | | CTGTAGAAAGATAAAGTGTGCTTGAATGAAGATAAATAATACCTTA |
| | | | | ATTGAATGGTTCGGAAGAAGAGCTTGCTGTTATTTGTCGAGGATAT |
| | | | | ATTTTTTCTATATTGTCTGCTTGTCTGTGTTCTGGGATGGCCATCCAT |
| | | | | TTGACTGCAAGTTATATTACCATGATGATCATGAATAAGGATCAAAT |
| | | | | AATATCCGGTTGCAACTATTCTCCCGTTTTTACTTTTAGGAAAGATAA |
| | | | | TTTAGTTGATATTTAAACCTTTCTGATGCAATAGTTGTCAAAAGCTCT |
| | | | | TATCCTGCTCAAGCCTCATTGCCACCTGCCAGGTTCCTCACATGCAA |
| | | | | AAGATCCGCACCTAGATGCTCTAGGCTTTTGGTGCTATAGGCTCTAG |
| | | | | GTGCTCTAGGCGTGCGCCTCACACCAAAATTAAGTTGTTTTTGGAAA |
| | | | | TATAACAATTTAGGTGAGTTTAGACTTTATGGTAAAAACATAATTAG |
| | | | | GAAATAGAAATTAAGAAAGAAAAATCCGGAAAGGAAAAATATTTTT |
| | | | | GTATGGAAAATATTAATTATGGAGGAACAAAAGTTAGGAAATGGA |
| | | | | ACAATAAAAATGAGGAAATTAGAGAAACTACGAAAAAAATTAAAAT |
| | | | | AAGGAAGAAAATAGAATTGAGTGAGAGAAATAGGTATGAAAAGAA |
| | | | | AAGAAAGACACTTGGATGTTGATTTCACAATCAGCTACTAAACTACT |
| | | | | AATAAGGCATTAAAATAACTATTTAGGTCCTTACACAATTTTTTAACT |
| | | | | GCAACGTATATATATACAGCAAATTTACGGTGCTTCTTAATTTTTTAG |
| | | | | CTATTGAAATTCAATTTATTTATAGTTCATTTACCCCCTTACGCCTAG |
| | | | | TATGCGCCTTAACAATTGATTTGTGTATATGGTTTCTTATTTTTACTC |
| | | | | TACAGAACTTGTATTCTAATTCCATTTGTTTAATGAACTTGAACTCAC |
| | | | | GTGTATGGCTAAATCTGTATGTTAATTTTCAATTTAATTTAGTTCTCC |
| | | | | TTTAACTTGTGTGTGTGATAATTTTGCATTATATTGTTACATTATTAA |
| | | | | AGGCTTACTTCTTGTTTATGCCTTCTCAATCTAAGGGTGACGTATATC |
| | | | | CTGTGGAGCCTTATGATGAATTCTCTGTGCATCAAGTTCTCGATGCT |
| | | | | CATTGGGGAGTTCTCTATGATGAAGATGTAAGACATGTTCAAAGTG |
| | | | | ATCAGTTTCTATATTGCTTCATCTTTCATTATCATGTTACTTTCCATGA |
| | | | | TTGAACATCACATGCTCTATTATTAAAAATAGGAAATTTTGTTGGCT |
| | | | | TTGTTTTCTGTAACTTAATTTGGAAGTTGTAATATAATTATTTTACGA |
| | | | | GGTAGGTATAATTAAGTGGATAGCATAGGCTATAGTTATCTCACCTT |
| | | | | TCATTCTCATATTGACTTTATATTTCCTTTGTTGTTTTCAATTCAGTCT |
| | | | | AGTGGACTTCGATCACATACGTTATCCATACTTGTGAATGATTCTCC |
| | | | | TGGTGTTCTCAACACGATTACCGGGGTTATGTCTCGGCGAGGTTAT |
| | | | | AACATCCAGGTCTGCATATTTCATGTTCATCTCTATTTTTCTTCCAAA |
| | | | | ATGTTTAGTGACTTGTCATTGTTGTTATTGTTTTTTTTCACTTTAATCT |
| | | | | ATTAGAGTCTTGCTGTGGGTCGAGCTGAGAGAGAGGGACTTTCTCG |
| | | | | CATTACAACTGTTATCCTTGGAAATGATGATACAGTTAGAAAGTTGG |
| | | | | TTCTGCAACTTCACAAGTTGATTGATATACATGAGGTAAGTGGTGGT |
| | | | | GAAATTAACTTTTTCCCTCATATTTGTAAGTGCATAATCAAGCAACAT |
| | | | | GTGACATTATAAGAAAGGGAAAACTAGGAAGAAAAATTTGACGAA |
| | | | | TACAGTGACTGGCTTTTATGATATCCAAGTTGAAACTGCAGTTTTCT |
| | | | | TGTGAGATAGTTTGAGCATTTTGTCCTATAAGGCTGTAACTGTTTGG |
| | | | | AAGAACCTGGAACAAATTTAAGCTTCAGTGTCGTACAATGTCATGG |
| | | | | TCTATATGTTTGCTAATGATTTATTCTTGAGGAGTAGGCATAATTTA |
| | | | | AAATATTATTGAAGTATAATCTTGTACCCATACTTTGGTGATCCGCC |
| | | | | CATGATAATTATACTTCATGTCATGCTTATTTTCTTTCCAAATAAAGA |
| | | | | ATCTAAAGTTTTATTATTTAACTATGGTGTATCTCCACAGAGTAATTT |
| | | | | GTGTGTGTGTGTGTGTCTTTTGTTTTTGACAAATTCATGACCCATTTG |
| | | | | AGTGAATGGATAACTATGGTGGGTGCTTGATTCAGGTACAAGATAT |
| | | | | TACACATTTGCCATTTGCAGAGCGAGAGTTAATGCTGATAAAGGTG |
| | | | | GCAGTCAATACTGCAGCCAGGAGAGATGTCCTAGATATTGCTAGCA |
| | | | | TATTTCGGGCCAAAGCTGTTGATGTGTCTGACCACACAATTACCCTA |
| | | | | GAGGTAGTGGCTTCCTATGCTTGAATGATTAGCTGGCTTGCTCTTTG |
| | | | | CTAATATAATTTCACAACTCAATAATGATCATGATTCACGAGCCAAT |
| | | | | ATCAGGCTATGCTCTTGAGTTCTCTCTATGTGTTTTTAGTGAAGTTG |
| | | | | AGTTATTTTGTTTGTTTGATCTTTTTTTATTATTGGCGTGGCAAGTTC |
| | | | | AAATCTATACCCTCTAACTGGCTTGAAATTTGAGTTCCCTTTTTTCTT |
| | | | | TGTGCTACGGCAGTTTATGCACGCAGTCACAATATATTCCGTTAGAT |
| | | | | GTCTAGTATAACTGTGTGAAGTTTGAAGTATTGATGATAGAATTGG |
| | | | | ATGGTATGTGATTTTGATTTTTCGACAATGCACTTCATATGGTAGCC |
| | | | | GGCCCCATTTAGCATGGGATATTAAGGGTTTGCTGTTGTTGTTGCAC |
| | | | | TTCCACCAAGGAAGAGCTTGATGCCTATTGATGCCTTTTTCTATCATC |
| | | | | TCCATCAGTAGCTGTTGTTGGTTTTCCAAATTTTATGACTTCACTGAT |
| | | | | TATGTTATCATTTGATAGCTTACTGGTGATTTAACAAAGATGGCGGC |
| | | | | CCTGCAGAAACTATTAGAGCCATATGGGATTTGTGAGGTCAGTCAT |
| | | | | TTCCTGCCAAGTTTCAACAAGTAAAGATGTTTGTTTGCCTATGTTTCT |
| | | | | CTAAACATTGACAATCTTTGTTTATTTTGTCAATTTCTGTTTTTATGTT |
| | | | | TACACCAGGTTCTTTATAGTATATATGTTCAGAACAGAAGTTTTTTA |
| | | | | AATCTGTTCCACTAACTTGGCATGTAAAGCTGGATGCCACTGTGAAC |
| | | | | TTTCAAAAGTTCCAACCCTTGAACTTGGCAAAAGTGTATCAGTTTGC |
| | | | | CCCCTCATTGCCATGCGTCAGCTTTTGATTGGAGAAAGGAAGTTCA |
| | | | | GAACAATGACATATTTGAAAACTCAAACCAATTAGAAGCTGACACG |
| | | | | TGGCATTGAGGGAGGGGGGGAAA |
| 1755 | Euphorbia | gDNA | 2403 | AAAATTAAAGAACTAATAAATGATAATAATGACGATTACAAACTAC |
| | heterophylla | Contig | | AAAAGTTTGAACAAACTAAATAATCAATTAAAAATACTAAAATTAAG |
| | | | | AAGGTAATTACTTCGTAAAAATTATAAATTTGGCTAAAATATTTTAA |
| | | | | ATTTTACTTGAGACTTGAGAATATAATTAATTAGTCGAAATTGGACC |
| | | | | TAAATAATAAAGAAAAACTACACTTAATAATCACTCATAATATAAAC |
| | | | | TCAACAAACACAAGGTCTCGAACATAACAATAATATAAACATTAAG |
| | | | | AATCTAATAAGTACTACATATTATACAAATTTATAAACTCTTTGCTTT |
| | | | | ATTCTTTTTTAATCTCTTCTGTCAAATATTTATTTTGCAATATATACTT |
| | | | | TTAAATTTTGTTAATTAATTTGGTTTGGTTTTTATCATGAAAACCGAA |
| | | | | AATTGAACCAAAACCAAACCAAAACCAAAGTATTAGAATTTTACAA |
| | | | | ATCAAAACCAAACCCTATAGATACATAAGAACAAACAATAAATATG |
| | | | | TTATATCCTAATAAATCGATTTGATTTTCTTATTGTTAAGATAAAAAA |
| | | | | TGCAAAATCACATATTACAAATCACAAGAGAAAAATAATAGAAGGA |
| | | | | AAAGAAAAGAAAACGCAAGAATGAACCCATATTCACGAACAACGA |
| | | | | TCTCTATTGAGAGAAAATGATAGCGAATTCTGGTTGAATGAGCTGC |
| | | | | AAGTAATCTTTCCTAATTTGTAGGTTCGCATATTTTGCTTAATTAGTT |
| | | | | TGTCAATTATTTTGTTTTAAAGACGCGTGTTTCTTTGTTTTCAATTAT |
| | | | | AAGTGCAATTTCATATTGTTTCCATTAAGGTGTAAGGTATGGTTACT |
| | | | | TTATTTTTTTTCAAAATAAATCATACTTTACCTCTTAACACAATTGGAT |
| | | | | CAAAATAGCAACCATTCATCTTATGGTGATAGAAACAACATAAAAAT |
| | | | | TACTTCGGATAACCATATAATTTCCCTTTCATTAATTATCAATTAATT |
| | | | | ATCGATGGTCAAAAGATCAATATCTAAGTTGTTTTATAAAAAATTTA |
| | | | | GGAGTTTTTAACAATTTGATGTACGAATGTTATATTTTGAAAATATT |
| | | | | ATTTAATTTTGTTAATCATTTCCCATTCTTCTCTGTTTCTCTTTAGCTCT |
| | | | | CAAATCTCTCCTTGGCACACTCTCAGTCTAATGGCCGCCGCAACAAT |
| | | | | TCAAACCTTCAAAACCTCTTCTTTCCCTTCCACATCTTCTGCTTCAATT |
| | | | | GCTTCTCTTGGCATAGCCAACTCCTCAATTACGATACCCCTCTCCAAA |
| | | | | TTCACTTCCAAGCATAATTTTTCCAAAAAATCTCTCAAGGTCTCTGCT |
| | | | | ACCGCTGCTGATGATGACGGAAAAATTTCCCATACCCCTTTCCCCAA |
| | | | | TAACGGCTCTCTCCCTCCAAGGCCCGTGTCAAAGTACGTTGCTCGCT |
| | | | | AATTCACTGCTTAAAGCTTGTTTCTTTTGAACTTATGTGATCATATAT |
| | | | | GGGCACTCTGCCATCTGGTTTAATTTGGGGGTTTTTCCTTTTGACGG |
| | | | | AATTTGAACATTTAAAATTGAGGGGTCATCAAATTAAGCTTTGGGAT |
| | | | | TTCTCAATTTCACATGTATTTTAGTGATTGTTCGTTATTGATTGATTA |
| | | | | TTGTTTGTTGCACATTGTGATGGGATTTAGGGTTAGGCGGCATACG |
| | | | | ATTTCTGTGTTTGTTGGAGATGAGAGTGGAATGATCAATCGCATTG |
| | | | | CTGGGGTTTTTGCGAGAAGGGGATATAACATTGAGTCCCTTGCTGT |
| | | | | GGGTCTTAATAAGGACAAGGCGCTTTTCACTATAGTTGTCTCTGGAA |
| | | | | CTGAAAGGGTGTTACAACAAGTTATGGAGCAGTTGCAGAAACTTGT |
| | | | | TAATGTTTTGAAGGTTGTATTCTTATTTCGAGGGAGTCCCTTTATCTA |
| | | | | CTGATTTTAGTTTTATAGTTATAATTGTTGGCACATAGTCCAGTTGAA |
| | | | | GAGAAATGGATACTGGATATATAGAAGAGACAGATTTCTATTTGCC |
| | | | | TATTTTCATATTATGGCTTTTGTTACATTGTTCGGATTGTAATTTCTCC |
| | | | | ATATGCTGTTGTTTACTATGATATTTAGGTTGAGGACCTATCGTCCG |
| | | | | AGCCACAGGTAGAACGTGAGCTAATGCTTATTAAACTGAAGGCAGA |
| | | | | TGCTAATAACCGTGCTGAGGTAACTGGATTTTCATCGTTGTTGAGTA |
| | | | | TGGAAATGCTTTGAGGATTCCAATTTGAATTGCTCCCTTGTTTTTCTC |
| | | | | GGCTACATTTTGTTCTTCGGTGTGATTTTCAGATTATGTGGTTGGTG |
| | | | | GACATCTTCAGAGCAAAAATAGTGGACATCTCTGAGCATTCAGTTA |
| | | | | CAATTGAGGTAAATGATTTGTTAATTGATTCCAATTCTAGAGATAAA |
| | | | | AAGATCCTAGTGGATGACCCCTCTGATACTTTAAAAATTAAAGA |
| 1756 | Euphorbia | gDNA | 1780 | TCTGATACTTTAAAAATTAAAGATTATTAATAGTTGTTTAATAATATT |
| | heterophylla | Contig | | GACAAATGAATGAACTGGAAATATTGAACATATTGTTGTTGAAAAG |
| | | | | TTGTGCATATTAATTATGAAGTTAAGTTTGATGCAAACTTGTAGCTA |
| | | | | ATCATTTTTCTAGTAATTTAGTTTCAATGATAGTATCAGTGTAATATA |
| | | | | TGATATGTGGAAATTTTTAAGGGCTTTAACATCTTAATTTTTCAGCAT |
| | | | | AGCTATGGAGTAATAGGAGTTCACTTTTAACTACCAAATATGTTTCT |
| | | | | GTTATGATTTTTTAACATTTTTCCTCATTTCTGATGCTTGTGTTGGTA |
| | | | | GCTATGCTTTTCATGATATACTAATTGCTATAGTTGTTTTAGTCTTTC |
| | | | | CATTACTTTTACTTGTAAAATATATTAAGTGGTATATTAGTTTGTTGA |
| | | | | GCTTGGTGAACCTCCATATTTTATTTATCTTTTATACCAAGTGCCCAG |
| | | | | ATTGTTTGTGGTTTATTATGGCTATGTTTTTTTTCCCATTGCATGGAA |
| | | | | ACCTTGGTGGCTTGAACAGAATTCTAGTTTATGTACGGTAGTTTTGA |
| | | | | AAAATGATGTGCGTTGAAAGTGTAGTTGAAATTTCAAATCAAATGC |
| | | | | ACCTTTTGAAATTCCTAAAGGCTAATTTATTGTTATTGGTTTAGGTGA |
| | | | | CTGGAGATCCAGGGAAGATGGTTGCTGTCGAGAGAAACTTAAGCA |
| | | | | AGTTTGGAATTAGAGAAATTGCAAGAACTGGAAAGGTTGTGTTCAG |
| | | | | TTTTCTTTTTATTGTGGCTGTTTTGTAGGTTTTAGTTTGCCGTTGATTC |
| | | | | GTGAGCCTGCTGTTCTGAATAGATTGCATTGAGAAGGGAAAAAATG |
| | | | | GGGGCTTGTGCTCCATTTTGGCGATTTTCAGCTGCTTCATATCCCGA |
| | | | | TCTTGGAGAAACAATGTCGAATAATTCTCTCTTGGAAACTAAAAGTT |
| | | | | CAGCAATTGTAGGGGAAGACACCACATCTTCAGGGGTATGCCAATT |
| | | | | TCAAGTTTTAATTTTGTCTGTTTGGTGCTTTTTAGAGACTATCCCTGA |
| | | | | ACATTTACAACTTTATGAGCAAATCCAGGGGGATGTATATCCAGTG |
| | | | | GAACCGTCTGACAGCTTTACCGTAACTCAAGTTCTCGATGCTCATTG |
| | | | | GGGTGTCCTCAATGAGGAAGATGTAGGTCTGCTTATTTTCAATTCCA |
| | | | | TCTTTTTTTTTCCGGTCTTATAGAGCGCACATTAATTTGTCGAGTATT |
| | | | | TCAGACAACTGGACTTCGATCCCACACTCTATCTATGGTAGTAAATG |
| | | | | ACTCTCCTGGAGTTCTCAACATCGTGACAGGGGTATTTGCTCGAAG |
| | | | | GGGCTATAACATTCAGGTATTTCTGATGACTTTCTTCTCTGGTTTGAC |
| | | | | CTTGTTTCCCTTAATCCTAGAATGGATTGAAGAACAAATGATTTTCTC |
| | | | | GTCTTTGGCGGCAGAGTTTGGCAGTTGGGCATTCAGAAATTGAGGG |
| | | | | GCGTTCACGAATTACAACTGTGGTTCCTGGTACTGATGAATCCATTA |
| | | | | GCAAGTTGGTGCAGCAACTTTACAAGCTAATAGAGCTCCACGAGGT |
| | | | | CAGAAAACTTCACCAACAAGTGTCAATTTTTTTGAGTAAATAATGTG |
| | | | | TTTTGTGTTTTGATGTAATATTCTATTGTTTTGAAACAAAACGCCTCT |
| | | | | ATTACCAATCTAATGTCTCGGAGAAAAAAGAACTTTTTGGTCCCTGT |
| | | | | GCTATAAACTTTTTGGCCATTTGGTCCATGTGGTATACACTTGACCTT |
| | | | | TTTGCTCCCTATTCTATGCAATATATTACCTTTTT |
| 1757 | Euphorbia | gDNA | 666 | GGGGAATTTGGAATCCCACATTGCGTGGCTATGAGCATAAAATGCC |
| | heterophylla | Contig | | TCCTACATTGATGAACAATCCTGACCCCATAGAATACCTTTTTGGGT |
| | | | | AATATATGGATGCTTGCAGGCTTATATCCCTTTCTTCAAAGCAATTTA |
| | | | | AAAACTAATATGTATGCTTTTGTTGCTTTAAATATTGGATCATTATGT |
| | | | | AATTAGCTATAAATTCCAGATTATGTGGCTGTTGGATGTCTTCAGAG |
| | | | | CTAAAGTTGTTGATACCTCAGAGCATACAATGACTATTGAGGTAATT |
| | | | | TTAAGTATTACGTATCATATCCTTTATCTGTACGTCTAATGGCTGTGA |
| | | | | AGATTCATGTAGGATTGGATTTAAAATTGAAAGGTCGAATATGGGA |
| | | | | GTTTTTCCTTTGGTTTGTGGCTTTGTGCCTTTCAACATCATAACTTTTT |
| | | | | ATGAGGTTTTTATTGTTCATCAATTTATCATATGATTGCAATGGGTCT |
| | | | | TAAGGTGAACCTTATCAGAAAATTGCTTAAGCAAAATATCTTGCAGT |
| | | | | TTATAATTGAACTAGTTCACTTGATGGTTTGGAGTCAAAGGGTTATT |
| | | | | TTGACTTAAAAACAGTAATGGTTCACTCGAATCCAGCGACGTGCTTC |
| | | | | TTTCTATTTATAGGAATGTTTATTTTTTTTGGTAGAGGACGTTTTCAA |
| | | | | TTC |
| 1758 | Euphorbia | gDNA | 351 | ATTTTGCTTTGAATTCTGTTTCCAGCTTACCGGAGATCTCGACAAGA |
| | heterophylla | Contig | | TGGTCGCATTGCAAAGGTTGTTGGAACCCTATGGCATTTGCGAGGT |
| | | | | TTGTGGTTAAAACTGTCTTTTCCTTATGCCATAACATCTTTTTAAAAA |
| | | | | TTATATCTTTAATTCTTTATCTTTTTATAGGTGGCAAGAACAGGGCG |
| | | | | AATAGCATTGGTTCGAGAGTCAGGTGTGGATTCTAATTATCTCCGTG |
| | | | | GATACTCGTTTCCTAAATAATAAATAAGCCACACTTGTTATGGTAAT |
| | | | | CCCCGAGTTTAAACTGGCTTTTGGCTTTTGTATCAGTGTTAACAGTT |
| | | | | GAGTGCTCTAGACACATTGTTT |
| 1759 | Euphorbia | gDNA | 318 | GAAAAATTGTTTGTGCTCAATAGGTGGAAGATATCTCTAAGGAACC |
| | heterophylla | Contig | | ACATGTGGAACGTGAACTGATGCTTATAAAACTTAATGTGGATCTA |
| | | | | GAAACTCGTCCCGAGGTAATCTTGTAGTATTTAGTTTGTCTCTTTTCT |
| | | | | TTCATTTATTATTTTGTAAGCCTTAGTTTTGTTGTATGATATTGACAA |
| | | | | TCTTCTGCAAATTTCTGCAACATATTGCGAAAACAACACTACAGGCT |
| | | | | CTTTTGAATTTGTTATGCATGCAAAATGTTTGATTTGGTCTACCTGCT |
| | | | | TCCAATCAATAGTTAATATGCATTCACAATTTATG |
| 1760 | Commelina | cDNA | 591 | ATTAAGTCTACAGCTGGATCATTCAATGGGAACACAGAACAATCAT |
| | diffusa | Contig | | CTGGAGGAGATGTTTATCCAGTGGAACCTTACGAAGGCTTTGTTTTC |
| | | | | AATCAAGTTCTTGATGCTCATTGGGGTGTTCTCGACGATGAAGATTC |
| | | | | GAGTGGCCTGCGTTCACACACATTGTCCATTGTTGTAAGTGATATTC |
| | | | | CCGGTGTCCTCAACATTGTAACTGGGGTGTTTGCTCGTAGGGGCTA |
| | | | | TAACATTCAGAGCCTTGCTGTTGGCCCAGCTGAAAAGGAAAATGTC |
| | | | | TCTCGTATTACTACTGTAGTCCCGGGAACAGATGAATCTATCGGCAA |
| | | | | GCTAGTTCAGCACCTTTACAAGCTTATCGATGTTCACGAGGTTCATG |
| | | | | ATATAACTCATTTGCCGTTCTCTGAAAGAGAGTTGATGCTTATAAAA |
| | | | | GTTGCTGTGAACACTGATGCTAGACGGGATATCCTAGACATTGCTA |
| | | | | ATGTTTTCCGGGCAAAAGCTGTTGATGTTTCTGATCATACAGTCACA |
| | | | | CTTGAGCTTACTGGAGACTTCGAGAAGTTGGTTGCATTACAGAAAC |
| | | | | TGTTGGAGCCTTATGGCATCTGTGAGGCGGCG |
| 1761 | Commelina | gDNA | 760 | CTAGAAGTTTATAAACATCACCTTGGCAATTTTTATCGTGACAAACT |
| | diffusa | Contig | | TCTTGTTTATTTATTTGGTTTTGCTTTGATTAAGTTGGTATTCAGCTA |
| | | | | GGAGTAACAGATTGCCAAATTGTAGAAATGCATTTTTTTTAATACCA |
| | | | | AACATTGGATATATAGGAAAGTTTTGAATATTCTTTCGTTTTAAGTT |
| | | | | AGTATGTTGGTTCTTTCTTTATTAGAGCCCTTTTAGTGCATTTCTCTC |
| | | | | GAAATTCTTATGAGATAATTGAGTTCTGTGTAACTGTAGAATAAGG |
| | | | | AGACACACTATATCTGTTTTTGTTGGAGATGAGAGCGGAATCATAA |
| | | | | ATCGGATAGCTGGTGTGTTTGCAAGAAGGGGATTCAACATTGAATC |
| | | | | ACTTGCAGTTGGTTTAAATAAGGACAAGGCACTATTTACTGTAGTTG |
| | | | | TGTCTGGGACTGAGAAAGTGTTACAGCAGGTTGTGGAGCAGCTAA |
| | | | | ATAAGCTCATTAGTGTTTTGAAGGTAAGTTGACAATTTCTGATTATT |
| | | | | GGTTAGAGTGCTCTGTGTTGATTTATAAGGATGTATTTATATTTATTT |
| | | | | GCACATGCTGTGGATTAGGTTGAGGATTTGTCCAAGGAGCCTCAGG |
| | | | | TTGAACGAGAGTTGATGCTTATAAAACTCAACGTTAACCCAGATGA |
| | | | | GCGCCAAGAGGTATGTACTATTGTATATCACTTTACAATTTGGTATT |
| | | | | TCACTTCTTGGAGTTCTAAATTTCCGGAGAATTGGCGAGTCTAGTAA |
| | | | | CTATTGGGCCAA |
| 1762 | Commelina | gDNA | 374 | TTATAACACTGTTAGTTGCCAAAACTTCTAGAACTAAACTTTTTACCT |
| | diffusa | Contig | | GCTATATGGATTACACTAGTAAATGACATAATGCTATCGCTTCTGGT |
| | | | | AAACCGTCTCGCAACTTGTCAATCACTTCATGAAATTTGACACCTTCT |
| | | | | ATTATGTAGTATCAACCATAATTTTGTGGTAATATTGCTTCTAATGTG |
| | | | | TTGCAGGTGGCGCGAACTGGCCGAGTTGCACTAGTTCGTGAGTCGA |
| | | | | GAGTTGACTCTAAATATCTGCGCTATGGCTATGCTCTTCCATTATGA |
| | | | | GCAACTTCAAAAACTGCACATAACCAGTGAGACCGGTAAAGATAAT |
| | | | | GCTCCTTCCTCAACTCATTTCCTAAATAAGTCAATCCCGTCTTT |
| 1763 | Commelina | gDNA | 310 | CACTCATAACTTTGGATTTTGATAATCTGATTTTTCTGTAGATTGCTT |
| | diffusa | Contig | | TGAGACGTGAGAAGATGGGCGCAACTGCACCCTTTTGGCGGTTCTC |
| | | | | AGCTGCTTCATATCCAGATCTTGAAGCCTCAGTGCCAGTTAGTACTC |
| | | | | CTATTAAGTCTACAGCTGGATCATTCAATGGGAACACAGAACAATC |
| | | | | ATCTGGAGTAAGTACTTGATTCATGTATGAATATATAATGTAACTGG |
| | | | | TTGAAAATAGAATTTTTATTGATTGTTTTGATGTGCGTGATTCTTTTT |
| | | | | CAAGCATCTGAGTTTGTAAGAGTGAGAA |
| 1764 | Commelina | gDNA | 288 | CATTCATGTATTTGCCCTACTGTCAATTTGCAGGTCATGGCTCTGGT |
| | diffusa | Contig | | CGATATTTTTAGGGCAAAAGTAGTTGACATTTCAGACGAGACTCTG |
| | | | | ACCATTGAGGTCATATCTATCTAAAAATCGTTTTCTTCTAAGAAAGT |
| | | | | GAACGTTAATTATCTTAGCTCTCCAGTCATGAGTCAATTCTTCTTCAG |
| | | | | CTGAGATCTTGGTCAACGTGCAGGTTACAGGAGATCCAGGGAAAAT |
| | | | | GGTTGCAGTTCAGAGGATCATGAACAAGTTTGGAAACAAAAGTTGA |
| | | | | AATATGAA |
| 1765 | Commelina | gDNA | 255 | TCATCAGTACTCTATTCGTACATGCATCTCTTGTACACTGCATATATC |
| | diffusa | Contig | | CTTTGATTTTCATTTTTATTGCTGCTAGGCTTGATTATAATTTAGATC |
| | | | | CCTCATTTTTTGTGGTTAAGGGAGATGTTTATCCAGTGGAACCTTAC |
| | | | | GAAGGCTTTGTTTTCAATCAAGTTCTTGATGCTCATTGGGGTGTTCT |
| | | | | CGACGATGAAGATGTAAGTTTCTGTACTATCATGTTTTAGTTTTCTTT |
| | | | | CCTTTGCTGTGAATGAT |
| 1766 | Commelina | gDNA | 210 | AGTCCCGGGAACAGATGAATCTATCGGCAAGCTAGTTCAGCACCTT |
| | diffusa | Contig | | TACAAGCTTATCGATGTTCACGAGGTCAATTGAGCTTTACATTCAGC |
| | | | | CATATTCTTCTACCGATTCTTGTATCTGAACATATGATGCATTCAGGT |
| | | | | TCATGATATAACTCATTTGCCGTTCTCTGAAAGAGAGTTGATGCTTA |
| | | | | TAAAAGTTGCTGTGAACACTGA |
| 1767 | Digitaria | cDNA | 569 | TGATAAATCGGATTGCAGGAGTTTTTGCAAGGAGGGGATATAATAT |
| | sanguinalis | Contig | | CGAGTCCCTTGCTGTTGGTTTAAACAAGGATAAAGCTCTTTTTACTA |
| | | | | TAGTTGTATCGGGAACTGAAAGAGTGTTGGAGCAAGTCATGAAAC |
| | | | | AACTTCTAAAGCTCGTTAATGTTTTAAAGGTTGAAGATATATCAAAG |
| | | | | GAGCCACAAGTAGAACGTGAGTTGATGCTCATCAAGATAAATGCTG |
| | | | | ATCCAAGATACCGTTTAGAGGTCAAGTGGTTAGTGGACATATTTAG |
| | | | | AGCTAGAATTGTTGATATTTCAGAGCACTCTATAACTATTGAGGTTA |
| | | | | CTGGTGACCCAGGGAAAATGGTTGCGGTGCAAAGAAATTTAAGCA |
| | | | | AGTTTGGGATCAAAGAAGTTGCTAGGACCGGGAAGATTGCCTTGA |
| | | | | GAAGAGAAAGAATGGGTGAAGATGCTCCTTTCTGGCGGTTTTCAGC |
| | | | | AGCTTCATATCCTGACCTTGAAGAAATGAACAAAAGAACTCCTCTTC |
| | | | | AAGCCAAAAAGAGAATGGAGTATCAGGAATCTGATAAGCCTGCTG |
| | | | | GGGGAGATGTTTATCCA |
| 1768 | Digitaria | gDNA | 5819 | GTGCTTGCCCCCCGTCGTCCCGCGCCAGCTGCGCCGTCGCCGCGGC |
| | sanguinalis | Contig | | GGCGTCGTCTTCGGGCGCTGGAGGCGGCGAGGCGGCCTCCGCGGT |
| | | | | CACGGCGGTCGCCCCCGCGGCCGCCGGCAGGGACAAGTAAGCGCT |
| | | | | CTCCCTCCCCACCCCGCCGAAAGTGTGGCGTCGTTTTTTATAATTTCT |
| | | | | CGCGGGGCGAGGTGGTGGCGCGGTTACTGTTCGTGGACCCTCACT |
| | | | | CCGCTTTTTTCCCAAATATTTTGTTTTTCCCTCCTCATGACGGGGCTG |
| | | | | GTCGGGCTTCTGGCGGCAAATGCAGGGTGCGGCGCCACACGATTTC |
| | | | | GGTGTTCGTCGGGGACGAGAGCGGCATGATCAACCGGATCGCCGG |
| | | | | GGTCTTCGCCAGGAGAGGGTACAACATTGAGTCCCTCGCCGTGGGT |
| | | | | CTCAACAAGGACAAGGCCCTCTTCACCATTGTCGTCTCCGGGACTGA |
| | | | | TAAAGTGCTCAACCAAGTCATCGAACAGCTCAATAAGCTTGTCAAT |
| | | | | GTCCACAGCGTGAGTTGGCCACACTAAAATTTCAGCAAAGTCGTGT |
| | | | | GAATTCGTACTTGATTACTTGAATTTCCCCTTTAAATTGGTAGCTGGT |
| | | | | GGCATTTCACTAATGCATTGAGATTTTTGTTTTATGTGTTTTACCTTG |
| | | | | CTGAATGCCCTTCCCCCTTTTAGTCTGTTAAGATAATTTCCACTTTTG |
| | | | | TGGAGCATCTTTTGTATATATAGAGTAGTACACAAAACCTTTTGCTT |
| | | | | TTCTGATTCGTCCATAATCCTTGTTTTCTTTTTTGGTATTCTGTTGCTT |
| | | | | TTCCCCATAGAATCACTGCTTGACAGTCATGTCAACATCAGGGACAT |
| | | | | ACTTCTTTCTAGGTGATACCTCAAATGTGTGTTGATACTATATATGCA |
| | | | | CTGTCATACACTTTCTTTGGGTGCACATACACTCCTTTGTCTTTTCTA |
| | | | | GGTGACGCCTCAAATGTGCATGTTATTATTCTGGCTGTGTAATGCCT |
| | | | | CAGAACTCAACAGTGACATTGTCGTATCATGTTTCCTTGGTTAATCA |
| | | | | TCGCCAAAAAATGACTTTGTTATTTTGACCATACAGTAGCTAATTAG |
| | | | | CTCTAATGCTATCCTGTTGAAAAGTCTATCTATAAATATAGAGCACC |
| | | | | CTTTTGGTTACAAGTGGGATGTTTCTGAATTTTCTTGTCTCTTTGCAG |
| | | | | TGTTGATCTCTTAGGGAAAAGTCTCTGAAAAAAATACTATGCAGAT |
| | | | | ATTAAATAGCTTAGGTAAATGTGCTTCATGGATCATCATTACTGCAC |
| | | | | TGTTTGTCTCAAGTCTCAACTCTCAACTACCGTTTAGATGGGCTTTAA |
| | | | | TATTTGTACTATTTTATTTTTCAAAAAATATTTACTACTATTGATATGT |
| | | | | TGCAATAAGAGTAATACCAGTAAATATTTATTATTTTTTGTGTCGAC |
| | | | | CCAATTAATTACAGGTCGAAGATCTATCTAAAGAACCTCAGGTTGA |
| | | | | AAGAGAGCTGATGCTTATAAAATTAAACGTTGAACCTGATCAACGG |
| | | | | CCTGAGGTATGTTATGGTGTTCACTAAGTGTTTGTAATCCAAAAGTT |
| | | | | GAATAGCATCCTGTATTTATGATACTAAATTACATGGTGAGTCTTAC |
| | | | | TGCAGTGCTTCTTTTCATGACTGACAGGTCATGGTTTTAGTTGATAT |
| | | | | TTTCAGAGCAAAAGTTGTTGATATATCTGATAACACACTTACAATTG |
| | | | | AGGTAAAAAAAACTCAAATCTCTAACTGGTGCACATATGTTAATTG |
| | | | | GTATCACTTTTCATGCGATGAACTATGTGTTTGATGGTTATTTGACC |
| | | | | ACTGTTAGGTAGCCGGAGATCCTGGAAAAATTGCTGCAGTCCAGAG |
| | | | | GAACCTAAGGAAATTTGGAATCAAAGAAATTTGCCGAACGGGAAA |
| | | | | GGTAATTTCTGAATATTCTTTCCTTACATGGATATGAATATTTTTTCG |
| | | | | TAGTACTAATTTTTAATGCTCCACCTTCTTAGATTGCTTTGAGACGTG |
| | | | | AAAAGATTGGTGCAACTGCACGTTTCTGGCGATTTTCTGCTGCTTCT |
| | | | | TATCCGGACCTTATTGAGGCACTGCCTAAACAACCACTTACATCTGT |
| | | | | AAATGGGAGAGTTAATGGCAGTTTCGAGCAACCATCCAATGCTGGG |
| | | | | GTATGTTCCCTTTGCCTTTTCCCTTTTTATTATGACCTGCTCTGTTTAT |
| | | | | AATTCACAACACTTAGTTATGGTGAGAGGATGCTTCTTTATTTCTTG |
| | | | | AACGATGCATATAAGCAATTTGAGGTGGATAAATTAAATAAAAACC |
| | | | | TGTAATGTCAGTAATACCTTTTCAGCTAACCAAATGTAGTTAATACC |
| | | | | TTTTCAGCTAACCAAATGTAGTTGAACTTCTATGTTAAGATAAAAGT |
| | | | | GCCAATAATTACCTATTGTGTTTAAGCAATTATCTGCAATGATGCAG |
| | | | | GGATATTAGTTCACTAAACCTAATGTGTTTGAAAAATAATTAACTTG |
| | | | | TCTTGTGAGGGGTTTGGGTCACTTCACCAAACATTTTTGCCAGTGCA |
| | | | | TGATACTACTTTTAGAAGTCAGAGTGCACTATAACAGTGCTTTTATG |
| | | | | AGTTATAAAGCAGTAATTGAAGGTACATATTTCATATTTGCATGCAT |
| | | | | AAACAGCTCTCCTAATGGACGAGTCATTACACAACTGGTATGTCAA |
| | | | | ACTTGTGCATATCTTCTATTATGCTAATGAGGGAACATATTGTTGGG |
| | | | | TGTTGCCCACCATAGTGCACAAACTTACATAAACAGCATTCCTAATG |
| | | | | GGTGAATCATGCAGTCCTAGCCATATTTCACACTTGCCGTGAAACTT |
| | | | | GGACTAATGCTAAATTGCTAATATCATTAATGAAATTGTGCAGCCAT |
| | | | | TGAATTTGTGTGATGCAGTATTACAATCTAGAGACTTCTATAACATG |
| | | | | TGATGTTTTGACCTTTGAACTGTTCAGGGTGATGTTTATCCTGTGGA |
| | | | | ACCTTATGAGAGCTCATCACTGAACCAAGTACTTGATGCCCATTGGG |
| | | | | GTGTTCTTGATGATGATGATGTAAGTTTATAGATTTCCATCGTGGGA |
| | | | | TTGTGGTTAGTGCTATATGGACTGTATTGTTGTAAGCATAATTGCAC |
| | | | | CCTGAATCTAATTCTAATGTGACTGGAGTGAGTTCTCATTTAATACTT |
| | | | | TCATAATGTTTGCAAGAAGTCAATTGTAGGCCGCCTATATAACTTTA |
| | | | | AGCAATGGTACTGCCTAATCTGCATGGTACTAAACTGGTACTGATTT |
| | | | | TGTTTGTTACAGTCAACTGGACTACGCTCACATACCCTCTCCATCCTT |
| | | | | GTGAACGATTGTCCTGGTGTCCTCAACATTGTCACAGGGGTCTTTGC |
| | | | | TCGCAGGGGCTACAATATACAGGTTTTCCTTATTGAAAAACTATCTT |
| | | | | TCTTCTTGTGAAATATAGTGCCTGGGTAGTTATTAATGACAGTTACT |
| | | | | TCTCATCAGAGCCTTGCTGTTGGCCCAGCTGAGAAGGAAGGCATTT |
| | | | | CACGCATTACAACTGTTGTTCCTGGTACTGATGAATCCATTGAGAAG |
| | | | | TTAGTTCAGCAGCTTTACAAGCTTATTGATGTGCATGAGGTAAGCA |
| | | | | GCATGTGACTTTATGAAACTCATTTTTTATTGCAATTTTATTATTTAA |
| | | | | CATAAGCATCTAAATTGTGCAGGTTCATGACATCACTCACTCACCTT |
| | | | | TTTCTGAAAGGGAGCTGATGCTTATTAAGGTTTCTGTAAACACTGCT |
| | | | | GCTCGGAGGGAAATCCTAGATATTGCTGAAATCTTCCGAGCAAAAC |
| | | | | CTGTTGATGTTTCTGACCACACAGTTACACTACAGGTTAGCTTCCTA |
| | | | | ACAATTTTGTCTCTCCTGGATTTACTTCAGTGTCTGTCCGAACACTGA |
| | | | | TCATAGCAGAGCCAGTTGTGATACAAGTTTTGTGCACAATCAAAAA |
| | | | | ATTAGATCATGGTGTTCTTTTACTTTTTTCAGCATAGCACTCTTGTAG |
| | | | | TAACAGTAACATTACCATATAAAAACACTGTGATGCCAATGACATTA |
| | | | | CTATTCTTCACCCTGCTTTATCTCACTCCTTCCAAGCTATTGGCGTATT |
| | | | | GCAAAACTCTTACATAATGGAGTTCTTATTGGTAATTTGCAGCTCAC |
| | | | | TGGTGATCTTAACAAGATGGTTGCATTGCAACGGTTATTGGAGCCTT |
| | | | | ATGGCATCTGTGAAGTATGTGTGCTTCTTATACAGTCACTGTCTGTT |
| | | | | ACTTTATTGTTTTTCTTATGTAGTTATTATGAAACAGACATTCCTTTC |
| | | | | ACAGTTTCACTAACTAGTTGTGAAACATAATCTAACTACTGTAATAA |
| | | | | TGTCGATCAAAGCATTATGCACAACATTGCACATGGCTAGCCCTTAG |
| | | | | GCCTTAGCAGTTAGAGAAAAAGGAAAATCTTGTAGCAGCTACTAAA |
| | | | | GGATTCCCAATCAGTTGCACACCTGTTTTATCTTTAGGCCTCCTCCAA |
| | | | | TGGACCGACTCTTAGCTAGCTCATAAATATTTTATTCGATGAACAAT |
| | | | | GCTCGCTAGCTCTTAGTTAAGAGATCAGCTCTTAGCCCAACCCCCTC |
| | | | | ACATGCATGTTTTTCCAAGCTCTTGTTCTACTGTGTCGAGCTCTAAG |
| | | | | AGCTAGCTAGTTGGCTCTTGCCATTAAAGGAGGCCTTGGATTGATA |
| | | | | CATTTGCTTTCCTTTTCTCTGCAAAGCAATTGCTCTTTCCTCACCGTTG |
| | | | | TGTTGCACTCATGACAGGTTGCCAGAACAGGTCGTGTGGCTCTGGT |
| | | | | CCGCGAATCCGGAGTGGATTCCACGTACCTCCGGGGCTACCCCCTC |
| | | | | CCATTGTAGCCTGGCGTTTGTGATGATGGCCCAGGAAAGGCGTGCC |
| | | | | TGGTTGCTAGAGCAGAATGTGTAGGCGTCCCTTGTTCGGTTGTGTG |
| | | | | TATTTGCTGGACTTGTTTGTTTCATGTTCGTCGACTCCTTGATTCGCT |
| | | | | TGCTTAATAATATGCTCCTTGGTGGATGCAAGCTGAGGTTGCATTTG |
| | | | | TATCCCGTTGACAGTAATAATAAGATGTATGTGCATCCTCGTAGTTC |
| | | | | AGTTGCCAGTCTTCAGTTGGGAGAGATGTAGCCAGCCACCGTCCAA |
| | | | | AATTAACTGGCCCGCGGCCCGCACCTTCCTATTATTCCTTCTGTCTG |
| | | | | GTGTTTTTAGCCGGGAGAAATTGTACATCTGGAGAAGGGACATGGC |
| | | | | TGCTCCGGGCTCTGGCTAGCAGCGTGTGCAGCCAGGAAGCTGTTGG |
| | | | | TAAGGGCGTCCACCATGGGAGGCAAAACCAGTCCATAGTGACTAA |
| | | | | AATATTTCATTCAACCTAGCTGACACATTGTGGAAGTAGTCCATATA |
| | | | | GTAGTCCATAGTAAAAGGTACCTATAAGCTTATGGGCTGCCCATAT |
| | | | | GAAATAAAAAAAAACAATTTCTCTCTCACATACCTCTCATCTCTCTTC |
| | | | | CTTTTCCTCTCACCACTACTGCCTACTTTTGGATGACCATTGTGAAGT |
| | | | | TTGCAATAGTTATGAAGTTTACAATAGTTATAGAGCACTTTCGTAAG |
| | | | | GTTCCACCACTATGAATTACTTAGTCCGAAAGAGGCTGCGGCATGC |
| | | | | CGCCGATTCGTTTTTGACCGTTCTATAGACTGCGTCTTGCGAGTCTC |
| | | | | CATGGCCCGAGACCTCTGTGTGGCCAATAGATCGATGGGCATGGG |
| | | | | GCTGGCTCCTGGCGTACACGCAACGACTGCATTTGCGGAGACGCGG |
| | | | | CGCATGCGTCGTGCGTGATCACGACGAGAAAGGAGACGAAACGAA |
| | | | | CGAGAGCGTGGGCCGAGAGACTTCGCCCGCCATCGCTTTTGGGCTT |
| | | | | AAAAGGAAGAGCCCAGTGGCCCACTACAGATCGGACGATGAGGAC |
| | | | | GACGGCGTGGCATGGCATCCGCCCATCCGGTATTCCGGCAGGAAG |
| | | | | GTACGGGAAGAATCGGACTCTCGCCTTCGACGACCAGGGCGGGCG |
| | | | | CCGATCCGAATC |
| 1769 | Digitaria | gDNA | 5263 | TTTTAATACATTATGATGTGGCAAATTTTTAAATCTTTGGTATTCACT |
| | sanguinalis | Contig | | TCAGAAAAATAATGTGTAAGCTGCCAAAAATAGGGAATGTTTCTAT |
| | | | | TGTACATTGTTAACATGTGTTAGCAACTGTCATCAGTGATCCTGTTC |
| | | | | GAAAACATGTGGCAATCAGTTTTATTGCCTTAGTGGGGATCTTTATG |
| | | | | TTGGAGAAAGTATTTTATTTTAGCTCATATAAATCATAAAAAGTGAG |
| | | | | AAACTTTCAAAATCAGGTAACTGTCAGGACAAAAAACATTTTATTTT |
| | | | | TTTATATTAAAAACAAGCAATCTTCCATATATGTTGATTTTGTACTAA |
| | | | | GGTTGTGAACCAAAACACAACTTCAAATCTGTCCACATTATCAGGTT |
| | | | | CGTTTTTGTCAATATAAAAGTAACTATTCTTTTTGTCACATACTATGC |
| | | | | CCTGTAGTGGTGTCCAGGAACTTAGGGAATCAGTAATTACTGCATG |
| | | | | TCCATACAGCAAAGTAGCATTCTTATTTTGTAAGATCTGTAAATCGT |
| | | | | TCCATACTGATACTGTAGCAAAACGTTATTAATTGTTCCCCTAGTAA |
| | | | | GTAATTATTAACACTTTCCCTTTTTAATTTATAGGGTTGTAAAGCGTC |
| | | | | ACACACTATCAGTTTTTGTTGGTGATGAAAGTGGGATGATCAATCG |
| | | | | AATTGCTGGGGTTTTTGCTAGAAGAGGATATAACATCGAGTCATTG |
| | | | | GCTGTTGGGCTAAACAAGGATAAAGCATTATTTACAATAGTAGTGT |
| | | | | CAGGAACAGACAAAGTATTGAACCAAGTTGTAGAGCAACTAAACAA |
| | | | | ACTTGTTAATGTTATAAAGGTCAGTATTTTTTCCTCTTAACATGTGGC |
| | | | | TTACAAAAATGATTAGTATGTCACAGAAGTCATCACTCTTTGTCTTG |
| | | | | TACTGAGACTGTTGAACATAGTATCTTATATTCACATCTTAACATTTG |
| | | | | GATTAATAAACAGGTTGTTGACTTATCAAATGTACCACAAGTTGAAA |
| | | | | GAGAACTGATGCTTATAAAAATAAATGCGGAGCGAGAGAAGCTGC |
| | | | | CTGAGGTATAATCATACATATCTTTCAAAACGTATCTGTTCTGACCTT |
| | | | | GTAACCTTAAGAATGAATATTGGATATTACGTTTGTAGAAAGAAAT |
| | | | | ATATAAATTGATAACAAAGATATCAGAAATCAGGTCTGTTGGTGCA |
| | | | | AACTAGCACATTAAACTTCATTGTTGGGGTAACATCTATAGTTGTGA |
| | | | | ATGCAGATAATTGTTTTGGCTCGCATTTTCAAAGCAGAAGTGGTTGA |
| | | | | TCTTTCAGACGATACACTAACTTTGGAGGTAATGTCGTTGTTGTGCC |
| | | | | TTGACTCAAAGATAACTCAATTTATCGTTCTTATGGTTCTTTCTTGTG |
| | | | | GATATGTTCAAGGTAACTGGAGATCCAGGAAAGATGGCTGCACTAC |
| | | | | AAAAGACTCTGAGCAAATATGGGATCATAGAAATTGCTAGAACTGG |
| | | | | CAAGGTCATTATTGTTCAGAAAATAGTGAAAATTTATTGTGATCTTG |
| | | | | TTCTACACTATATCTAACGAGCCTTAGTGATGTTATAGATAGCTTTG |
| | | | | CGCCGTGAAAGAATGGGAGAATCTGCTCCATTTTGGGGGTTCTCTG |
| | | | | CAGCATCTTATCCTGATCTCGAAGTGACAATACCTTCAAATTCTCGTC |
| | | | | TAAGCACTGGAATGGATGCCGTGAGTCAGAATCCCAATGAATCTTC |
| | | | | AGGGGTAAGAAACCACAATTGTCCTTTTATCTGATCAATGGATGTAT |
| | | | | GGCGTGTTCTTTAGTTTCTAAGCACCGCAGTGCTGTTTTGTAATTGT |
| | | | | AACTGATATTTCCCTATTTGTTTGCTTGTCTGCATCGAGTAGATTTGC |
| | | | | ACTCATGCTTTCTGTTGACTAGTTTCGGATGAACAAATTGTTCTTTCT |
| | | | | GAATTCGTTTACATTGCTGTTCATTCTGTTCAATTGGGTACTAAATAC |
| | | | | CTGTCTATCTTCATGAAGAAACAGCCTTATGGCTAATTGGCTATTGA |
| | | | | TAGTTTTGCAATATTGGTCCCTTCCAAGGTGAACCTAGTATGTGTGT |
| | | | | ACTTGTATATGACATCACTTGCATATTTTTATATTGAACATATCTGAC |
| | | | | ACATTCCAGTGCTCACTCAATACTTAATTTGTCGTTTGAACTCTTAAG |
| | | | | TATATATAAAATGCTTGCTCATCTTTTGTTATTGGAGTGTTGTATTTT |
| | | | | GAATGCTTGCTCATCTTTTATATAAAATGCTTGCTCATCTTTTATTTA |
| | | | | AAATGCTTGTTCATATTAGTTTCAATTTTGAATGATTGTTTTATACAT |
| | | | | GCTTTTCTTCTTCTACCCAGGGCGATGTCTATCCAGTGGAATCGTAT |
| | | | | GAAAGTTTTTCAGCAAATCAAATTCTTGATGCTCATTGGGGTATTAT |
| | | | | GACAGATGGTGATGTAAGATCTTGTATATTCTAATCACTTTGAACTA |
| | | | | TTTGATGGTAGCTAGAGATGATTTGCATCTAGTTCTACTCATCCTTTC |
| | | | | ACAAGAAGTTATTGTGTTGCTAATAGTTAACAATCAAGACTGCAAG |
| | | | | ATCCAAATTAAACTCTAGTTTGTATAACTAAATTATGGTTCACAGTG |
| | | | | AGAAGTACAAACCATACCATACAGCAGTTAAATGTACCTAATTATGA |
| | | | | ATTAATCGAGGAATTTCTTGTTGCTTCAGTCAATAGGGTTTTGTTCA |
| | | | | CATACTTTATCGATTCTCGTGAATGATTTTCCTGGAGTTCTCAATGTT |
| | | | | GTGACAGGTGTTTTCTCCCGAAGGGGCTACAATATTCAGGTTCTTCA |
| | | | | AAACCCATTTAGTATATTGTGCTCCCTTTAATTAGATCACTACAGGAT |
| | | | | CCAGTCCTTGCTAAACAGTAACCATCACTGTCACAGAGTCTTGCTGT |
| | | | | TGGCCCAGCTGAAAAAGAAGGAACATCTCGCATCACTACTGTTGTT |
| | | | | CCTGGAACTGACGAATCTATTGCCAAGCTAATACATCAACTGTACAA |
| | | | | GCTGATTGATGTTCTTGAGGTCAATAATTTGCAGTTTCCATGACTGT |
| | | | | TATAGAAGTAATGCAGTTGGTATAATTGTTATAGAAGTAGTGTGGC |
| | | | | TCAAATTATTTTTTGGTTTCTCAGGTCAAGGATTTTACTCATTTACCA |
| | | | | TTTGCTGCTAGAGAGTTAATGATCATAAAGGTTGCTGCAAATGCTG |
| | | | | CAGCTCGAAGGGATGTCCTAGATATTGCTCAGATTTTTGAGGCCCC |
| | | | | AAAAGTTGACATATCAGACCACACAATTACACTACAGGTAACTGTTA |
| | | | | TTGAAATAACTTTTATTTAAATATACCTCATATAGGACTATAAGATTC |
| | | | | TTCTGAAAGTATGATAGTTTAATCATCTTTATGTACACAATAACGAC |
| | | | | TTCATATTGTTCATATATGGTATTTTTTAATGCGTATTGTAGTATTGG |
| | | | | TCCAACCATGAAGGGACTAATATATTGATGTTAATAGGCCAAATAT |
| | | | | GTCAGGTATTAGCATATTTTAGCAATTGGCAATCTTTGAATAAAGAG |
| | | | | TTATTTGAAATTCGTAAATTTTGGACTCATCATTTTCTTTTCTACTTTG |
| | | | | CCTTTGACTAATGATCTTTTCTACCTGCACTGACTTCTTTAATCAATC |
| | | | | GTGTTTCATTAGCCGTGCCTAGTGAGTAATAATCTTGTTGGTCTGTG |
| | | | | CAGCTTACCGGAGACATTGACAGAATGGTTAGATTGCAAAAGATGG |
| | | | | TAGAGCAATATGGCATCTGTGAGGTTTGTGTTGACTATGTTGCTTCT |
| | | | | AATTTCACTGATATGTCTGGCATTACCTTACCATTTCATGCATGATGA |
| | | | | TTTTCTTTTTCTCACAAGTTATTAATTGCCAGTGTTACTTCAGCAACC |
| | | | | TCAAGGAGACACTTGAAATATATTTATGTAATTGTCTTTCCTCTTTGC |
| | | | | CTATGCCTACTGCAGGTTGCACGAACTGGCCGGGTTGCTCTGCTCC |
| | | | | GTGAGTCTGGAGTCGACTCCAAGTACCTCCGTGGATTCGAGCTGCC |
| | | | | GCTGTAGTTATCCATTCACTGACATACCACTACGGTTTTACTCCGTGC |
| | | | | TTCCATTTGGTCTTCTGAGCATATGATGCAGCTTTTCATGTTTCCCTT |
| | | | | TGGACCAACAGTAGAAGAAATATAACTCAAAACCAAAGGTTGGGA |
| | | | | ATCATAGGCGGAATATTCTGTACAATCGTGTTAATTGGCTAAACAGT |
| | | | | GATAGGTTATATGGGTTGCTATGGGCTTATGCTGTAAGTTTCTTGAT |
| | | | | TTTGTAAGTAGCACTCATTTGCTCAGTTAAAAATTGAAATCTCACGG |
| | | | | AGCAAACGGGAGGTTTGTGGAATCATAAATGAACCTACCAAGCATA |
| | | | | ACAGGTGGGTTGGCATTGGCACAGCTAGCGCAACCGATCAAATGA |
| | | | | GAGATCAGATTGTGCGAGTTTTATTGATATAGCAGAAAAAGCTAAG |
| | | | | ACTATTACACTGGGAGGCCATATAGGTGGAAAGAAATGTATAATTT |
| | | | | TGCTCCAAATAAACAGGTATAATTTAATTTACCTGTCACCTTTTCAAA |
| | | | | TATACTCGGCACATTTACTGTATTCTGACGATTCAAGAATCTACGCT |
| | | | | CGTTGTATCAATATCTTTTTGCTATTTTTGGACTAGGTCGAGCTAGC |
| | | | | GGAAGATTTTTCTCTGGGCTGAATGTTCCATCCCAAGCCCGACGGA |
| | | | | CCTTCTTTAATTTGTGTTTCGTGTCAGACCATGATTTGGGTTTATTGG |
| | | | | GGTGGGATTCCTACCCATGATTTGGGTTTATCACTTTCTTGTTCGGTT |
| | | | | GGAGGGTATCCAATACAGGGGCTGATTTAATCCCTGCCGGTATTGG |
| | | | | GTGGATCCCTGAGCTGCCACCCAATCCCTGTGGTGTTTCATTCCCTG |
| | | | | TTAGCACTAATCCCCTCTCCTCCCTTTTCTTGTTCGGTTACCCACCAT |
| | | | | GGATCAGAAGACGCAACAAGTGATGGTAACAGGTAGAAACATACA |
| | | | | TATCCCTTTCCTGGACAGAAGATCATACGCAATATGGCAAGCTCATA |
| | | | | CACAATAAGAAGACTACCTGGTCAATTGTTTCCTGGACAGAAGTGC |
| | | | | ATACATAACTCCACCAAAGACTGACAACAGGGTAATCATGACCATC |
| | | | | AAGTTTGCAGACTAACCAACAATCGTTAAGTACCAAGCAATCTATG |
| | | | | ATCATGGAACTATAGCATTGTTTGCCCCTAATTCTAACCAGCCAAAA |
| | | | | ATCCAAGACTACTCAACTGTTAGAACACCGATAATAAGCTGGAGCA |
| | | | | ATGAGTATCGATAGGCTCCTTAACAACATATAATCTTAATGCTTAGG |
| | | | | CTGCATATTCTTCTTGGCACCAAACTTTGTAGGGGATTCAGTGCACT |
| | | | | GAGCTCAGTCTAGGAATAGCCAACATTGTCAGACCAAGCTTGTAGT |
| | | | | AGT |
| 1770 | Digitaria | gDNA | 4483 | ATAAATATAAAGGGCCGTGGCCGACTGAGGACGAACAACTCCAAA |
| | sanguinalis | Contig | | CGAATATTTGCATATTACTTTTATCTCTCAAACCCTAACTTTTCCAATC |
| | | | | TTTTGCTTTCCTTCTTCGTCTCCACGGTGATTCAGAAGGTTCTAAGTG |
| | | | | GCCCTGTCAACCTTAGAACAACCCTAGGTTCGTTGCCTCCGACGAG |
| | | | | GTCTCTCCCGAGGCGGCGATCGTAGGCTTTTGCAGCGACCTAGCTA |
| | | | | CAGGCGGTCTAACCGGCTGCGCAGAGAGGCGCCGGCGAGGTGATT |
| | | | | TCAACTTTCGCGCGATCCAGCGCACGCGTTTGTTGACCGAAAAGGC |
| | | | | GTCAACACTTTCTGATTCTTAGATGAAATAAAGCCTGCAAATGAACT |
| | | | | TTTTTTTTCAGTCCAACAATTCATTTCAACAGAAGTAAGATCGGTAG |
| | | | | TTGACAGTACAGACACAGAAGACTTGAATTGAAACTAAGGTTCCTG |
| | | | | ACGTGCAAATTATATATGGAGACCTGAATGGCAACACCATTTCCTTG |
| | | | | TTTGCCGATTTCAGAACATGAATAGCCATCGGGATGTCTGAATGATT |
| | | | | CAGTTTCATGTTCAACAAACACATGTCTATTTGGTTAACTTACGATGT |
| | | | | ATGTCTATTCAGAACATGGCAATATCAGAACATGAATTTGCCGGCG |
| | | | | TGCACTCTCCAGTCCGTCCGGGGCGTCCACCTCCTTAGCTTGCCGGC |
| | | | | AAAGGGCAAAGTTGCCTCCACGCCAGCCACACTAGAGTGTCCACCA |
| | | | | AAACAGGAAGAAGCCGCCGGCGGACTTCCATTCAGACACGCCTGG |
| | | | | GGACTTGAGGTGCTCATCGAGGGAGATGCCGAAGAGAGATGACGG |
| | | | | AGACACTGAGAAGAAACCGCCGTCCGAACTAAACGGATCGGAAGT |
| | | | | TGAACTTGGATGGGCAAGGGTAGGGCTATAGGGTTGGATCCATTC |
| | | | | GGGTCACCCATAACAAAGTAATTAAAAAATAATTATTTAATTAGATG |
| | | | | GGTTGCTTTAAGACTCGTCTCCTAAAATAAAAGTGGAGTATCGGATT |
| | | | | AGTTCTCTATTATAATTAGTAGCAAATTAAATATATATGTGCATTGCT |
| | | | | GCAGGCTAAAGAAATGTATCACTACTACACAACCCCTTTCGCAGCC |
| | | | | GGTTTTGCCAACCGTTTGATTCCACCAACCGGCTATGATTAGGGTAC |
| | | | | CCAGGATTTTAGTTCCACATCCAAACAATTGAAAAAAATTTACTACC |
| | | | | CGAGCAACATCAACCTATGGCGAAGTGTTATTAATCATTCCCCTAGT |
| | | | | AAGTTATTATTAACAGTTTTTATTGCACTTCCATTGACATGCCTGGAC |
| | | | | AAATTTTCCTTTTTAATTTGTAGGGTTATTAAGCATCACACACTATCA |
| | | | | GTTTTTGTTGGTGATCAAAGTAGGATGATCAATCGAATTGCTGTGG |
| | | | | TTTTTGGTAGAAGAGGATATAACATCGAGCCATTGGCTGTTGGGCT |
| | | | | AAACTAGGATAAAGTATTATTTACAAAAGTAGTGTCAGGAATAGAC |
| | | | | AAATTATTGAACCAAGTTGTAGAGCAACTAAACAAACTTGTTATTGT |
| | | | | TATAAAGGTCCGTATTTTTCCCTCTAACATGTGACTTACAAAAATGA |
| | | | | TTAGTACGTCACAGAAGTCATCACTCTGTCTTGTCCTGAGTCTGTTC |
| | | | | AACATGGTATCTGATATTCACATGTTAACATTTGGATTGATAAGCAG |
| | | | | GTTGTTGTTATGTTCGGCAAGATGTATCAGAGGAAGAAGTGTGGCT |
| | | | | GAGGAAGAACGATTGACAAAAACGTGAAGCACTGCCCGTCAACCG |
| | | | | ACAACGTACGGCGACATATGCTACAGTAGGTGCGACAACGGCGAG |
| | | | | ACACGCTATAGTAGGCAGGTGCGCAACTACTGTAGGCCAACGCTCC |
| | | | | CACGATGTGGAGTAGTTCAGATTACTTTTCCTATACTTTAGTAGTAA |
| | | | | TTGCCAGTATTGTAATTTGGATATATGTCTCTGTTCGGAATTGGAAA |
| | | | | GAGTTATGTTTAGAGATCAATATGATCTCTAGACGTCCTGAGCTGCT |
| | | | | GTTTCTTCGTCTCACTCTTGCTCCCACCGGCCGTTAGGACGGCGTCT |
| | | | | CGTCCTCGCCGCCGGCGTGAACCCTCACCTCGCCTGCCAACTTCCTA |
| | | | | TTCATACGCACTCCAAGGGATTAGCATTGCCAACAATTTGGTATCAG |
| | | | | CAAGCAGCTGTGTCCAGGGATTGACGACTGGGAATTCCATCCACAC |
| | | | | CGCCGCCATGTCCTCCACTCCCGTGTCCACCACAGCACCGCCGCCTT |
| | | | | CCACTACCGCACCACCACTCTCATCCACCAACCTCGCCACCACTACT |
| | | | | GTTCCAGTCGCCACCATCGACACCCTAACGACGGCGATCTACAGCTT |
| | | | | GCAACGTCAAATAGGAGAATTCGCCAACCGTCTCTCTATGGTTGAA |
| | | | | CATCGTCCTCATCCACCGGAGGCCGGACCATCGGCATCACTGCCAC |
| | | | | ATGGGTTGCCGGGCTATGACGGCATCCCACATCATCATCCATAATC |
| | | | | GTCCATACCTCCACAACCATCGAACTGGTTCCCATCACTCAGATTGC |
| | | | | CTTTTCACCCTCCCCTTCACCACTTACTCCACTGCATGAGAGATTCTA |
| | | | | CATGATGGAAAGAGATGCAGGTGGCAAATGAAGCATTATTGCAAA |
| | | | | AATATAATGATGTCAATATTCTAGTAATGATTAATGAAACTCTTATT |
| | | | | GCAATGGGAATCTTGATGATACAGTTTTTCCACCACCAAAGATACAT |
| | | | | GTCATGCTTTATTAATACTCCTTATTGTGGCCATGCACTTCATCAGGA |
| | | | | CCTTAGCTCGATGAAGGGCGCTGTGTGCAGTGTGTCTTCAAATTTA |
| | | | | GCAGTGCATTTACGTCGTATACACTCCTCTCTGCTGCTGTAAGTAGT |
| | | | | TCGACAAGGCACTGCATCGTTCCCTCGCCGCCGGACTGCAATGTCCT |
| | | | | CACTGACCGGCAACAGCTATCATCATAGGTTGGGAGTATACGGGAG |
| | | | | GGATTTGAGCGTAGGATGAAGCGGTTACAGACATTACGGACAATA |
| | | | | AAGTTTTTCTGTCTCTCTGTACATGGTTGTGGCTTGTGACAAAACCA |
| | | | | ACATCCACCACTCACAGGAACAAGAGAAACTGTAGCAAACACTAGT |
| | | | | AGGAGGAGAATAGCTGCTGCTTTCCCAGTTGACATCTTAATGCCCA |
| | | | | ATGTTATGTCTACTAGATATTGCTAAGAAAATGTTATGACTAGATAT |
| | | | | TGACCTCCTGCGTTCATACGTGCTCCTATATATAGGAGGCAAAGCAT |
| | | | | GCAGGTTGGTATACACCGGGTTTAATGCGCTTTTATCCTTGATACCA |
| | | | | TATTACGTGATCTAGATATGGTATCATTTATGAAGCGATCCTTAGAG |
| | | | | ATATTAATGCCTATTGTAGAGTTCCATGTTATCCTCTTGTATTCATAT |
| | | | | TTTATGACCATAAAATAACTAGAATTATTTGAATATCTCATCAGGAT |
| | | | | AAATAATCAGTTTATTAATTCATGCCTTACCTTTGTTTTGTAACCACA |
| | | | | GCAGGTCCCGTCTCCCGGATTGTCTTCTTTTGCATGCACGCATGCAT |
| | | | | TCAGGGCCGCTGACACCTCGAACGTTTCCATCCGTATATGCATGCCA |
| | | | | TCGCCCGGGCGACCATGTAACTCAGCTCTGCGCCGACCAATCAGTG |
| | | | | CACCTGCCTCTTGACAGCCTCGCCGCCGACGAGCCGCCGTGCGCGC |
| | | | | GAAGGTCGCCGCGCTCTCGCAGTACGTCGCCACGCCAACAAGTTCA |
| | | | | AGAAGGACGGGACCGGCGGCGGCGGCTACACCACCGCCACCGTCG |
| | | | | GTACGTACGGACGGTAGCCACGTCAGCACATCCATCTGCTTGATCA |
| | | | | TGCACAAAACCTGCATATACCTATAATTTGGAAGGTGCTAAAATAAT |
| | | | | ACAATTATGTGTGCAGAAGAGTGGATTTAAATCTTCAAGCAACATG |
| | | | | CAACAAATGTGAGAAAGAAGAGTGCTTTTAAATCTTCAATCAACAT |
| | | | | GCGACAAATTTTACCCTTATGAAAGCTGCGGGAGTACCCGCCGAGT |
| | | | | GCAGCCGTCCACGACAGTGAAAAAAAAAGTTACCGCCGGATCTGA |
| | | | | GCCTAACAGCCGGTAATTCTGTCTCCTTTTGTTGATGCAACCCGTTG |
| | | | | AAGCCCCACCCGTCGGTGCACCGATTAGATTCCACCGCTACTCTTCA |
| | | | | ACTTTTCATTATTCATCAAACTTTCCATTATTCATCCACTCACACCTAT |
| | | | | CACGATCTATAGTGTTTTCTCTCATCTACAGTGTATTTTCACCCAGTC |
| | | | | TAAACACACCCTTGAAAACACACCCGGTGTGGCCTCTCCCCATTCAC |
| | | | | CCAGAAGTGTTAAACACACCCTTGAAAACACACCCGGTGTGGCCTC |
| | | | | TCCCCGTTCACACAGAAGTGTCCCGAACTGCCACGGGGAGCCACTG |
| | | | | GCGTGGGCGGTAAAAAAAGAGAAAAAAATCTACCCCTCCTTTTTTT |
| | | | | GCCTTCCTCGCCACCTCGGCCGTGGCCGTCGTGATCGTCCGTAGCTT |
| | | | | CCCTCCGTGGCCGTCAGCAAGGTACGCTCCCTGCACTTCTTCTTTTCT |
| | | | | CCATGTACTC |
| 1771 | Digitaria | gDNA | 3928 | TCTTTAGTTTCTCAGCACTGCAATAATGCTTTGTAATTGTAACTGATA |
| | sanguinalis | Contig | | TTTTCCTAGTTGTTTGCTTGTTTGCATTGAGTAGATTTGCACTCATGC |
| | | | | TTTCTGTTGACTAGTTTCAGATGAACAAATTTTCTTTCCGAATTGGTT |
| | | | | TAATTTGCTGTTCATTCCGTTCCATTGGGTACTGAATACCTGTCCATC |
| | | | | GTCATGAAGAAACAGCCTTATGGCTAATTGGCTATTGATAGTTTTGC |
| | | | | AACATTGGTCCTTCCAAGGTGAACCTAGTATGTGTGTACTTGTATAT |
| | | | | GACATCACTTGCATATTTTTATATTGAACATATCTGACACATTCCAGT |
| | | | | GCTCACTCAATACTTAATTTGTCGTTTGAACTCTTAAATATATATAAA |
| | | | | TGCTCGCATATCCTTTTTTTTTGTTGCCGTGTTGTATTTTGAAAATAC |
| | | | | TTTTATCATCTTCATGATATTAGTTCCAATTTTGAATGATGGTTTTAT |
| | | | | ATGTGCTTTTCTTCTACCCAGGGTGATGTCTATCCAGTGGAATCTTA |
| | | | | TGAAAGTTTTTCAGCAAATCAAGTTCTTGATGCTCATTGGGGTATTA |
| | | | | TGACTGATGGTGATGTAAGATCTTGTATATTCTAATCACTTTGAACT |
| | | | | ATTTGATGGTAGCTAGAGATGATTTGCATCTAGTTCTACTCATCCTTT |
| | | | | CACAAGAAATTATTGTTGCTAATAGTTAACAATCAAGACTGCAAGAT |
| | | | | CCAAATTAAACTCTAGTTTGTATAACTAAATTATGGTTCACAGTGAG |
| | | | | AAGTACAAACCATACCATACAGCAGTTAAATGTACCTAGTCATGAAC |
| | | | | TAATCTAGAATTTCTTGTTGCTTCAGTCTACAGGGTTTTGTTCACATA |
| | | | | CTTTATCGATTCTTGTGAATGATTTCCCTGGAGTTCTCAATGTTGTGA |
| | | | | CAGGTGTTTTTTCCCGAAGGGGCTACAATATTCAGGTTCTTCAAAAC |
| | | | | CCATTTAGTATATTGTGCTCCCTTTAATTAGATCACTACAGGATCCAG |
| | | | | TCCTTGCTAAACAGTAACCATCACTGTCACAGAGTCTTGCTGTTGGC |
| | | | | CCAGCTGAAAAAGAAGGAACATCTCGCATCACTACTGTTGTTCCTG |
| | | | | GAACTGACGAATCTATTGCCAAGCTAATACATCAACTGTACAAGCT |
| | | | | GATTGATGTTCTTGAGGTCAATAATTTGCAGTTTCCATGACTGTTAT |
| | | | | AGAAGTAATGCAGTTGGTATAATTGTTATAGAAGTAGTGTGGCTCA |
| | | | | AATTATTTTTTGGTTTCTCAGGTCAAGGATTTTACTCATTTACCATTT |
| | | | | GCTGCTAGAGTTAATGATCATAAAGGTTGCTGCAAATGCTGCAGCT |
| | | | | AGAAGGGATGTCCTAGATATTGCTCAGATTTTTGAGGCCCCAAAAG |
| | | | | TTGACATATCAGACCACACAATTACACTACAGGTAAACTGTTATTGA |
| | | | | AAAACTTTTATTTAAATATACCTCATATAGGACTGTAAGATTCTTCTG |
| | | | | AAAGTATGATATTGAAGTACTGGTCCAACCATGAAGGGACTAATAT |
| | | | | ATTGATGTTAATAGGTCAAATAATGTCAGGTATTAGCATATTTTAGC |
| | | | | AATTGGCAATCGTTGAAGAAAGAGTTATTTGAAATTTGTAATTTTTG |
| | | | | GACTCAGTCAATCATTTTCTTTCTGCTTTGCTTTTGACTAATGATCAT |
| | | | | TCATACCTGCACTGACTTTTTTAATCAATCATGTTTCATTAGTACTGT |
| | | | | GCCTAGTGAGCTATATCTTGTTGTCTTGTGCAGCTTACCGGAGACAT |
| | | | | TGACAGAATGGTTAGATTGCAAAAAATGGTAGAGCAATACGGCATC |
| | | | | TGTGAGGTTTGTGCTGACTATGGAGCTTCTAATTCACTGATATGTCT |
| | | | | GTCATTACCTTACCATTTCATGCATGATGATTTTGTTTTTCTCACAAG |
| | | | | TTATTAATTGCCAATGTTAATTGTACTTCAGCAACCTCAAGGAGACG |
| | | | | CTTGAAATATATTTATGTAATTCTGTCTCTTTTCTTATGCCTACTACA |
| | | | | GGTTGCACGAACTGGCCGGGTTGCTCTGCTCCGTGAGTCTGGAGTG |
| | | | | GACTCCAAGTACCTCCGTGGATTCGACCTCCCGCTCCCCCTGTAGTT |
| | | | | CTCCATTCACTGACATACCACTCTACGGTTTTACGCTGTGGTTCCATT |
| | | | | TGGTCTTTTGAGCATGATGCAGCGTTTAATGTTTCCTTTGGGACCAA |
| | | | | CAGTATTAGAAAGATAACTCAAAACCAAAGAGTGAGAATCATAGGA |
| | | | | AGAATATTCTGTGCAATCGTGTTAATTGGCTGCAGAGATAGGTTATC |
| | | | | TGGATTGCTATGGGCTTATGCTGTAAGTTTCTTGATTATGTAAGTAG |
| | | | | TAAAGTAGCACTCATTTGCTCTGAAAGAAATGGAAATCTCACGGAG |
| | | | | CAAACGGGAGGTTTGTGGAATCAGAAATGACCCTACCAAGCATAAC |
| | | | | AGGTCGGTTGGCATTGGCACAGCTAACAAAACCGATCAAATGAGA |
| | | | | GATAAGATAGTGCAAGTTTTATTGATATAGAGGAAAAAGAATACAA |
| | | | | GACTAGCACTGAGGCTATAGGTAGGAAAAAACTACATAGGTATAAT |
| | | | | TTTATTCCAAATAGACAGGTATAATTTTACCCTTCACCTTTTCAAATA |
| | | | | TCCTTGGCATACTGTATTCCTCACGATTCCAAGAATCTACGCCCGTTT |
| | | | | TATTCATGTTCGCCTAAATGGCGGTGTGTTCGGTGTGTACGTTTTAT |
| | | | | TCAGTGTTCGCCTAAATGGCCGTGTATTCGGCGTGTACACAGTACAC |
| | | | | GGCGGTTAATTGTGCAATTTAGGGTATAAACATTAGTTTACATGGTT |
| | | | | TGCTTGTTTACACGGATTTACACGGTCCAAACAGCCTACACGGCACA |
| | | | | TTAAAAACACGGTTTTGCATGGCATGTTGGCATTTTCTTCTTCGTTTA |
| | | | | GGCGATGGAGATCCAAGAAGATTCCAATCTGAACCAGAAGATGTCC |
| | | | | TAACCAGCCTAATCTGAACATGCTTGTTAGTCCTATGTTAATCTATCC |
| | | | | GTTGTAATTTGTGGTAGCATATGACTTATGAGTTGTATTTCCTAGAT |
| | | | | AATGGATGACAGCTATATTTAATTACCTATCACTTTTTAATTGGTTAA |
| | | | | GTGAATAAATTGTCTAACCATGAGATTATATGTATATTTTTCTAATAT |
| | | | | AGCACAAAAACGTGTAATCCATAAATTCCGTCTAAACGGTCTAAAC |
| | | | | GCTACACGACACCTTTTCAATTGTTTAGTGTTTACGGTTTAAGCTAAC |
| | | | | ACTGGTTTTATCAATGTCTTTTTGCTATTTTTGGACTAGGTCGAGGTA |
| | | | | GCCGAAGGTTTCTTGTTTCTCTGGCTGAAAGTTCAATCGCAAGCCCG |
| | | | | ACGGACTGACTTTGTGTTTCGTGTCGGCCCATGATTTGGGTTTATCG |
| | | | | AGGTTGGGAGGTGCCTGCTACTCCCTCCCTCCCTCCTGCAAGGGATT |
| | | | | ATAGCCTAGCCCAACGAGTCAGAAAGAACTATATCAAATAATTCAA |
| | | | | TGAACCGATGTTGGCCCACAAAAAGACTAAATATATAAGCTGCTGC |
| | | | | TATTTGCAATTTTGATAAGGCTTTCATCCCGGGCTGGACCTGTGCGC |
| | | | | AGGTGTTAGGCTAGCTAAAGCTAGGTTGATTTTTAGTGTTTTTAGCT |
| | | | | CATGCAGGACAACTTATTTTGGAGGAACAACTCCACGTTGCTGCTAC |
| | | | | AAATAGATAGATAGGTAGATATGGCACATGTATTGGAGGAACCTCT |
| | | | | CCAATGTGCTAATAGGTAGAAACAGCACAAAGTGTTTATGAACCAC |
| | | | | TTCAACGTGTTGAGCTAGATATCATTCTAGAGATGAAGATAGACCA |
| | | | | AAAGGTCGGAATCCGAATCCACGGATCACATGAGCTTAATCCAAAG |
| | | | | CCCTTAGGATACAAGATTTAGTCCAAGATCTAAACGGTAGAACAAC |
| | | | | ATGTTCTATTCATAAGGTAGTGGGTACATCGAAACCACCTTACAAAC |
| | | | | TTTGGCTTGCCTCTATTTATAGGC |
| 1772 | Digitaria | gDNA | 579 | TTATATATCTCCCTGAGTGCCTTTCAATAGTTATAGTTGGATTGACTA |
| | sanguinalis | Contig | | ATAAGTAATAATTCATCTATGAATGACCCAAATAAAGGTTACCAGTC |
| | | | | AACAAATTAGTACTCACCCTTACAAAGTTGTTATAAATATCGTTAAT |
| | | | | CACCTCAATCCTTCCAGAAGTAACTCCTGTAAAACTCTGTCTAACCAT |
| | | | | AACTGTTGTCAGGCGATTTCCCACAAAAGAATTTGCGAAAATGTTG |
| | | | | ATAAAGAAACCAGAGCGAATTCCTAGTCTCCTATCCCAGTATTTCTG |
| | | | | AACTGACTTGGAAGGTTTGCGCTGGCGGTGGCTGATGCCATAACAA |
| | | | | AGATGGGGTCCGGAGATGTTAATTTTAGTCTTGTTTTATTAATTGGA |
| | | | | ATTTCATTTCATGATCCAGGTTAGAGATATTACACACCTGCCATTTGC |
| | | | | TGAGCGAGAATTGATGCTGATAAAGGTCGCTGTGAATACTACTGCA |
| | | | | CGGCGGGATGTCCTTGACATTGCCAGCATATTCAGAGCTAAGGCAG |
| | | | | TTGATGTCTCGGATCATACCATTACTCTTGAGGTAGAATGTAGGAGT |
| | | | | CATTCATTATACCATT |
| 1773 | Digitaria | gDNA | 402 | GTATCTACCCGAGATTCCGTCAAGAGAAGTATCAATGGTGACACTA |
| | sanguinalis | Contig | | GCACATCATCAGGGGTGTGTAAACTTCCATTTATTAAGATTAAGCAT |
| | | | | AAGATCATATCGTTCTTGCCAGAATAAGTTCGGTATAGTTCACTTCA |
| | | | | ATCCATTTTTGCATGAGTAAATTTACAATGCAGCACACTTCGGTTCC |
| | | | | ACCCATCATAGCCATTTTGCTTATAAAGATTGATGAAATGCTTTCTTA |
| | | | | GTCTAAAGGGGGATGTTTATCCAGTGGAGCCATACGATGACTTTAT |
| | | | | GGTGCATCAAGTACTTGATGCGCATTGGGGTGTCCTCAATGATGAA |
| | | | | GATGTAAGCATTCTTTTATCCGTTCCCAAATCTTTCGAGCTTCCTGCT |
| | | | | GTACTCTGAAATCCCGCCCAGGCGCGG |
| 1774 | Digitaria | gDNA | 227 | TATACTGTGTTCCTGCCAAACTGATTCATGGCTGAATGCATTCAGAC |
| | sanguinalis | Contig | | AAGTGGGGTCCGTTCACATACTCTATCGATGCTTGTCAATGACAAGC |
| | | | | CAGGAGTTCTAAATGTTGTTACGGGGGTTTTCGCTCGAAGAGGTTA |
| | | | | CAACATTCAGGTATGCAATCACTATCTAGCCTTGTGGTCTGGCGCTG |
| | | | | GGTCCTTTGTTGTTGTTGTTGAGGTTTGTGTGTTGTTGTC |
| 1775 | Kochia | gDNA | 12412 | TAGTGGAAGAGATGAAATGTCTAGTTATGGAATTATGGTTGATGAT |
| | scoparia | Contig | | TTTGGTTTCTGGTTTGGTAAACGGAGGTTTAATTCTCTGGCTAGTTT |
| | | | | TGCATTGATGAACAAAGTCATTGCCCAAGCCACCAACTGTTTTCATT |
| | | | | TGAAGGAGGCTCCCCGACTCATGTATTTTTCAGCCATGAATCTCGAT |
| | | | | CAAGTGATTAATTTGATCTTAGGTCATTCATATTGCTCTTGTTGTTCG |
| | | | | CATGGTTTTTTTACAACTTTCTAGCATTCAGACTATGGTAATTTCTTT |
| | | | | TTTCATGCCAACACTTCCATCATTTGTCATAACCTATTGCTTGACGAA |
| | | | | GATTCTGTGTAATGACATGTGCTTGAGTTTCCACTTATTCACAACAA |
| | | | | CCAAGTTGTGAGAAGTTGATGATTTATAGGGCGTGTTTGACAATAG |
| | | | | TAGCTGGCACTGGGCGTTTAGTTGTGGAAATAGTTGGTAGTGCTAA |
| | | | | CATTGGTAGTTAGCGGTAGTAGTAGATGTTAAGTGTCAACAAGGGA |
| | | | | TTATACAATCTAATAATTTGGCAATGGTAACAGTTGTATATATAATA |
| | | | | AAAGAAAAATGGTATAAGAAGAAAAAATTGTACCCGCTACATGACA |
| | | | | AATGTTAGTCCTAGTGTTGAGCAATATTCTCTTTAAGCACTTACCTCT |
| | | | | ATCCGAAACATTATTTTGCCAAACCATAATTTAAATTACCAAGTAGT |
| | | | | GTTTTGATCTAGTCATACCACTAAATGCTATCTTATACTCCTACGCCG |
| | | | | ATCACGCCCATAGCATAGGTGAAACTATATAAAAAAAAATGCTAAT |
| | | | | GAAATGCCCCTATCCCAAAACAAAATAAATGCATTGTAACCCAAGTA |
| | | | | TTAGTAGATTTGATCATGGTATTTGGTCGATTTTGATTAATGGCTGT |
| | | | | AGTTCCCAAGTGCAATAGGAATCTTCTTTGGTGAATGTACCCACGAC |
| | | | | CCTGAAAAGCCTTGAATTTTGCATTTATTGTAATCAATCAATCAATCA |
| | | | | AAGCCTTATTATCATATGTGGGGTCGAACATGAACCAAAAGTGATT |
| | | | | ATTCCAGGAGGTCATCCATATAGATTTTCCTCCTCTATTCTCTACGAT |
| | | | | CGAGAGCCTCACTCTTGCAAATCCAACTGTTTCAAATCCTTTCTAACT |
| | | | | CCTCCACTTTATGGTAATCATTGTATAAAAAAACAAAAAACAAAAAA |
| | | | | CAAAGAAAGAAAAAGAAAGAAAGAAAAGAAAGGAGATCAAAGGG |
| | | | | CATAATAGTTAATTATGATGCCAAGTAAAAAATTAGTCTGGGTCTTG |
| | | | | GGAAATAGTGATGTGACAATGAAGAGGTTAACGGATGATGGTTTA |
| | | | | TTCCACTGGAATTTGTTCTAATTGTTCGTCTTTCAAACCTTGTTTGAG |
| | | | | TTATTTAGTTGCCTTTTGGTGTTGTTCAAATCAATTTATTATCGTGAA |
| | | | | TTTGTGATGACTGAGATAGGTGTTTTCACAACGTATTTGGTCTAGAA |
| | | | | CTAGTCACAACTCGCATTTTGTTTGTGGTATGTGTAGTAGTGACGGT |
| | | | | GTTTCCACGGGAGAAACAGAGTGATGCTGCGTTTGCATGCATTATT |
| | | | | GTTTTTGGTATCATCTTTGTTTCTGATTCAATGTTTGGAATTGAAATG |
| | | | | GAGATTACTTCCTAGTTGTTTAAATATCTTATTTGTGATTCAGGAAGT |
| | | | | TGAGTTTGGTTTAAAATGTATGTATCTTTGGATTTTAGAAAAAGCCA |
| | | | | AGGGAGGTCAAATGCCCAAATGTGACTTGTGATGAAGGGAATTAA |
| | | | | AAGAGATATGGAGAAAGTAGGATCATAGAGCTCTGGATAGAGGTG |
| | | | | AATGGAGGAAGAGGTCCTTATTGATGATTGTAAAATATAAATTCTT |
| | | | | GATTCATGCAACCGTTATTCGTTAAACAATATCAAAGCTTTGTTATA |
| | | | | ATTCTTAAGGCTGTAGTGTTTCCTTGTAGAAATGCTGTAATCTGCAA |
| | | | | ATTTTGCACATTTAATGAATTATATGATGGGTTATCTTTAAATTGTCT |
| | | | | ATAATCTTAATTGTGGTTGTTTAAAATGTCTAAGGGCAAGACGACAT |
| | | | | ACCATTTCAGTATTTGTTGGGGATGAGAGTGGAATGATAAATCGAA |
| | | | | TTGCTGGGGTCTTCGCTAGAAGGGGTTATAATATTGAATCTCTTGCT |
| | | | | GTTGGATTGAACAAGGACAAAGCTCTCTTTACAATTGTTGTCTCTGG |
| | | | | AACTGATAAGGTTTTGCAGCAAGTGATGGAACAACTTCAGAAGCTT |
| | | | | GTAAATGTTCTGAAGGTTGTCTTTTGCTTTAGATGAAAATTAATTTT |
| | | | | GCATTGATGTTTTCCACGAAGTATTATCTGAGATCTATTCTCATGGC |
| | | | | CTTTTATTTATGTTTAGGTTGTAGATTTATCCAAGGAGCCTCAGGTA |
| | | | | GAACGTGAATTAATGCTTGTTAAAGTTGGAGCTGATCAGAGTAAAC |
| | | | | GTGCTGAGGTATCATTCACAATATGTTTCGGTTCCCCAAAAGTGGAA |
| | | | | GCTAGTAATATTATTCTTGCTTGTCAGAAAATTTCGTTTAGTTGTAGC |
| | | | | TAATTATTTTGAAATAGTAAATTTGTTGTATGCTAATCTTCCGCCATC |
| | | | | ATTCATCCTGATTGAACTTTACTGTACAAGTATCTTTAAATCTTTGAG |
| | | | | CTGAGTGAATTCTAATTTATAATGTTTGCTACTTATATAATAACTTTT |
| | | | | GTTATTTCCTTTTGATACTGCAGTTAATGTGGTTGGTGGACATCTTCC |
| | | | | GTGCTAAAATTGTGGATATTTCAGAAGAAAATCTTACGATGGAGGT |
| | | | | ATCACTGGAAACATTATGCAGATTAAGACAAATTTCTAAAGAACTAT |
| | | | | GTAATCATCCTAACGAGGATTGACTTCCAAGAATTTAACACATGTTA |
| | | | | TCTGCTTTATTTTTCAGTAGTCTTTGATGCTTTAGGTGCATTTGAATA |
| | | | | ATTTCTGTTTTAATATATATTCTAATAATGTTATAGTTACTGTATGTG |
| | | | | ATTCTATGATCCAGGAGATGTAACTTAAGTATTTTGTTTTGCTTGGT |
| | | | | GCATACCCATTTACTTCCTGGAAATATTTCATGTAGCTTGTGTCATTT |
| | | | | ATATACTTGATCAAACAAGGTCCGAAAGGAAGTCATTTGAGCAACA |
| | | | | ACTTTTAACAACCAAGGTCCCTTTTTTTTTTTTTTTTTTTTCTCATTCTA |
| | | | | AAACTCTAATCTCACAAACTGCAATCTTTTAGGAACAAAGGGAGAC |
| | | | | CTCCTCCACCTAATATTTCTTTTAAACCAAAGACATTTTAGGATAAAT |
| | | | | TGAGAAGCTTTTAGAACAAGAGGAAATCAGCAACACTTAATGTTCT |
| | | | | TTCTTGGTCATGATGGACATGAATGAGGAATCTTTGGATGATGCTCC |
| | | | | CGTAGCTTTTAGTCCTTATTTAACTTTATGTATTTTGGGTTGTTTTGA |
| | | | | GTCTTTGTCTCTCCCTTGGGTTTTGTTGTGTTTTTTAGCTTAATGGTC |
| | | | | CTTTTGCTTTTTAATTGGCTCTCTTTGACTTTTGTTGGTTTTGTCGTGG |
| | | | | GGTTCCTTGGGGCTCTTCTCCCCTTGTGTTCCCTTGTACTCTTTTACTT |
| | | | | TTGGTAATGAATTTCTTTATATACCAATTATAATAGTAATAATAATAA |
| | | | | TAAAGAACCAGTGACAATAGGTTTAGTCACCAAAAGTTTTTAGACTT |
| | | | | GCAACATAGAATATTATACACATTAAAACACAATAGTAAAACTAAA |
| | | | | ACTAAAACGCCTACCAATTGGTGGTTTCTTCTCTTTTGAGAACTTTTC |
| | | | | CAGAGCATCCCAATCTCTTATTGTAGCAAGTGCAAACACCTTAAGCC |
| | | | | AATACCATCTTTTCTCAGAAACCTGGAAAAGAGTGGCAATGTCAAG |
| | | | | TAGACAATTTAAACAAAGTAGCAACTCGTCGACAAAAGAAATAAGG |
| | | | | ATTGAACGAACATGTATCTTGTTGCTTATAAAAAGAAGAAACAAATT |
| | | | | ACATACCTTGAATTCATTTTTCACCCTCAAAGCAGCTCGATGATTTCC |
| | | | | GAGTACAATGCATGTGCGAATCGTGTCACTGATGCTTGAATCTACA |
| | | | | AAGATTGCTTGCTTTGTGGATACCTCAAGCTCATGCTGCATTCTATT |
| | | | | ATGGTAGAAAAATTAAATTTTAAACACACATGAACAAGAAAATTCA |
| | | | | TAAACTAAAAGCTTATATTTAATCTTGTTCAGTTATGCATTAAGTGA |
| | | | | AAATATCACCCCCACAGGATACAAGGAACAAAAACAACTGAACATG |
| | | | | AGAGATAAAGGGCTGATAATTGCGAGATGACAATAAAGATGACAA |
| | | | | TAAAAGAGCAAGATAAATAATCTTGTTCCCCACAGGATACAAGGAA |
| | | | | CAAAAACTATTGCGAGATGACAATAAAAGAGCTCTTTTATTGTCATC |
| | | | | CTTTATCTTGCTCTTTTTCTGCGTTCAGCAAGTTTTTTCCATCGTTATT |
| | | | | TGAGATGGATGTGTGGGAATACTATGAGAGATAGAATCAAAAATG |
| | | | | AAGTTATCTATGGGAAAGTTGGTGTTGCAGATATTGCAAGTAAAGT |
| | | | | AAGATGAAGGAAAACCGTTTGCATTGGTTTGGTCATGTAAGACGGA |
| | | | | GATCTCAAGAGGCACCGGTGCAAAGAGTAGAAAACTGGGATCAGA |
| | | | | GTATTTTTAAATGAGGTAGAGGGAGACCGAGAATGACTTGGTGAG |
| | | | | AAGGAGTTAAAAGGGATCTACAAGCGAATGAGGCTCTTGATTGTA |
| | | | | GGGGGTGGAGGAGGAAAATATTTGTTGATGACTTCCCTAAATACTC |
| | | | | ACTTTTGGTTCATGTTAGTCGACCCCACTTATGGGACTAAGGCTTTG |
| | | | | ATTGATTGATTGACTTGTCGTGCACTGTTTATTTTTTTATAAAAAATT |
| | | | | CTTTTCTTTTATTACATGTTTTAAAAATTATTCGACAAATCCTTTGGT |
| | | | | GAATTACATAAGAATTTATGCTATTATCTTGGTGTATCTCTTATGTAA |
| | | | | ATAATTTTCCATGTTATAGCTATGTTTTTTGATCAGCTTTCTCTATGTC |
| | | | | GCACAAGGTAGAACCAAACCAAGAGTTCCGAGGATTGGTGTGTGA |
| | | | | AGGAGCTAATGTTCTCCCTTCCAAGTTTGTAGAGGATTCACTTCGTG |
| | | | | ATGGATATGGTAGGATTCTCTCTCTCTTTGATTTTTATATTGGAATAC |
| | | | | CTGACCCTTCTCTCCATTAAATTAAATTTTGTCATCAAGCTGTGCAGG |
| | | | | AAAAGTTGTCAATATTAGGCATTTGGATAAAGTAATTGACTCCATCA |
| | | | | ATGGGTGGTACATGGAGCGAGGCCTCTTTGGCATGGTTAATATCTC |
| | | | | ATTTCTTTCTGTTGTTTAAGACTTCTGGATCAGCCTCTTTCATTGCCT |
| | | | | ATCCTTATTTTGTTAAGGCTTGAGCGCGGCAATGTTTTGGAGCAGG |
| | | | | AGATTCAATGTTTTATTCCTACAGGTTTCAGGTGTTGAGATCCTTTCA |
| | | | | GGAGGTATACTAAGGTTACAAATTGCTGAAGCTGAGGTCAATGATG |
| | | | | TTTCAATTCGTTTCCTTGATCGGAAGACGTAAGTGATAATTTTCATAT |
| | | | | TTGGTACTAAAACGAGTGAAAAAGGAACAGTGGAGTCTCGATCATC |
| | | | | TCATAGCGTTTGTTTAATCAAAAGTACATTACTTCGGGCCTGTTCTT |
| | | | | GTCAGCTTACTAAACTTGAACTTATTTGAATGGAATTTGGTTGAATG |
| | | | | TATTATATCGATCCTATATTTGAGTGCTTCATGAGACCTTATTGGTCT |
| | | | | TAAACTCAACACTTGAACTGAACTTATTGGACCTAATTTTTGAATGT |
| | | | | AAAGAGCACACATTCTTAGTGTTAAACATTTTCATTAATTTTACGACT |
| | | | | GCATTATTTAATTTGGAAATGAAGAGGGTTTGTCCGTTTGTGGTTCA |
| | | | | TTAATATTGATACTACGTGTACTTATTATTGTTCTCTTTAGACGTGAG |
| | | | | CCAACTGCGGGGAAGACTAAGCCGGAAACAATACTTCGACAACTTA |
| | | | | CAACCAAAAAAGGACAGGTAAAATGCAATTTGTATGTCTGTTACTG |
| | | | | TGGTAGTAGACTAGTAGTAAATGATGTTCCACCTTTTCTGATGTATT |
| | | | | GATGTAAGATTAGGGTATCTACTGAGAGAATAATTTTCCTAGAGCTT |
| | | | | TATATGAAGTATTAATTTTTTATTGATTTTTAGTCTTTTTGTCTGCTGA |
| | | | | AAACTACTGAATTTCATTACATAGTTCTTAGATTTATATCAAACAAAT |
| | | | | GAGCAACCAGTGATTTGTGATATTTTAGTATGAAATCTTCCACGTTT |
| | | | | GGAAGATCAGTTTTCAGTTACTTGTTTTAAAATCCATAGGTTTTTATT |
| | | | | CTGCTATGGGTCCAGTGATTTAGAGCAGAAAAAAAAACATAACTGA |
| | | | | AAGAATAAAAGGGGAATTAGTTGATAGTTTGATGAAATAGAAGGG |
| | | | | GAGTTGCCTTGAGATCAACGTGATCTATACAATCACTCACTGCTCAC |
| | | | | TTTTTAGAGGTGGAGCTCTCCAATAGTATAAGCTAAGAGTAATTAAT |
| | | | | TTATTGTTGCATTCTCTCTCCTGATCCTCCTTTCATTTCCTTATCTGAT |
| | | | | CTATCCTTAGTGTTTTTTATTTTCATTCCCGACCATTGTGGCCTTTCGT |
| | | | | AACGTAAATGATTAATGGTGGTATTTTGTGTTGATAATTTTCATTTCA |
| | | | | TTGGTTTATTGGCCGATTTATTTATGATTTGGTAATGAGAAATCATA |
| | | | | CAATTTTGAATTTGCCAGTGTGTTGAGGTCTCACGTATCCTATATGC |
| | | | | TTCTGCCGCAGGTTTACAGTTTAAATCAGGGAAAAAGGGATGTAGA |
| | | | | AACTGTTTTAACAATGGGAATTATGGAAGATGTTAGCATTTTCCCCC |
| | | | | AGCCTGCTGGAGGTAACTGTGTCCTGTATTCTTATTAGCTTTATCAA |
| | | | | CTGAAAAAAATGCTGGTTTTTTTTAATTTTTTTTTAATTTTTTATTGGT |
| | | | | AGATTTGTATTGTTTACGCTTTTAAGGTTTAACCATTTAATAAATAAA |
| | | | | TCTGTTTGTAAGCCCTTTGTGTTTAATATCCCTTTTCGAAGCTTTTGTT |
| | | | | GGCCGCTGAGGCACTAAAGATGCTCTTTCTTTCTGTGTATTCCTCTTT |
| | | | | AAGAGGTTACCCATGTATTGGTGAGTCTGTATAGGCTTTGGTTAAAT |
| | | | | TCATACACTATCAAATTAGGAAAAGAAAAAGAAGTTATCGAGTTTA |
| | | | | TAACTAAAGAGTTTGCGAAAGGCACACTCGTATTCACCCAACCAAC |
| | | | | CTTGAATTTAAGTTTGAAGGAGATGCAAAACTAAGTGATAGAGGCT |
| | | | | CATATAATCATATAGACCACTTTGCCTTGTTGTAAGCCCATGTTGGC |
| | | | | GTAGTTTATGTTTTCCATATCCCAGTTTGGATGATTCCATCAATTACA |
| | | | | GTTTTCGAGATGAATTGTCATTTTGCAATTTCACACCTGCAATAACC |
| | | | | ATTGTGTGGGCCTGTGGCTATGGGCAATGGACTCACAAGTCACATT |
| | | | | GTACTGACTTTCCTTCTGAAAGCATAGAGGACAATTGATTTCGCGTT |
| | | | | CTAATATTATGCTTTAACAGAGTCAACAGAGATGATGAGATTGTTTT |
| | | | | ATTGTGCAACATCTCTCCTAGCCTGAGATTCTTTATGTCGTCAAATCG |
| | | | | CTCTATTGGTCTTTACTTTATTAGATCCTTTAAAGTGAAAGATACCCT |
| | | | | TTTCTATATTAATTTTTGTTTGAATGGAGTCAAGCTACCTAGGCAATT |
| | | | | TGATATATGATGTTACCGTCAACAAAATTGTTTGGCTTCTGTAGTGT |
| | | | | TTGTTTTTTTGGATGGAATAGGCCTCTCTTATCTGTATCCACTTCATT |
| | | | | GCTAAAAAAGATTGTTTATGTTGGATGAGAAGTTTGAGTTTTGCATT |
| | | | | TGTTCTTTGTTAATAAAAAAATATTAGCTGTTGCCTTAAACCTCTGGA |
| | | | | CGAATAAAAAATTTAGTCTAACATATATCTTTTGCTTCTAAAATTCCA |
| | | | | GACACTGGTAAGGTTGATTTGGTAATGAATGTGGTTGAGCGTGTGA |
| | | | | GTGGAGGTTTTTCGGCGGGTGGTGGAATTTCAAGCGGGTAAGAGA |
| | | | | ACATGTGACTCTGCTCTGTGCAATTTCTTATATGATGGTCAATGTTG |
| | | | | CTATGCATATGTTTATTCTGATCCAACAAATGCATTTACAGGATAAC |
| | | | | GAGTGGCCCATTGTCAGGTCTGATTGGAAGGTATATATCTGCTCTTC |
| | | | | CTATTTCTGTCGGATAATCTATTCAACTTCTCATGTAAATGCCATACA |
| | | | | AGAAAAAGCATTTCCTCTTGGTCAATTTCTATGTGATAATTGGGTTT |
| | | | | AAGGAATTTATATACTCGCATGCACACACACACTGACACACATACAT |
| | | | | CAAAGCAACATCTTTTTTGATTAATGATTTCTTAACATGCTGTGAACT |
| | | | | AATACTTTCTGTTTATGTTAGTTTATGTGTGCAAATTTTATAATTAAT |
| | | | | AATAGCTTTTGCTTTAATTTGGTTGCAGCTTCGCATATTCTCACAGAA |
| | | | | ATCTTTTTGGTAAAAATCAAAAAGTAAATGTTTCACTTGAGAGGGG |
| | | | | ACAAATTGACTCTATATTTCGGATAAATTACACAATTCCATGGATTG |
| | | | | AAGGTGATGATAAGCGTACACAACAGTCAATAGTGATTCAGGTGAT |
| | | | | TGGCATCAGTCTTGGCTGTCTGTATATCTTTTTAGAAAAAGCGGGTA |
| | | | | TTGCTTATCTTTCTTTGTGATTTTGATTCTCTAACTTAACTTTATTGGT |
| | | | | CATCCAGAATTCAAGAACTCCTGGTACTTTGGTCCATGGAAACCAAC |
| | | | | CTGGAAATACTAACATAACCATTGGCCGTGTAACTGCTGGCATGGA |
| | | | | ATTTAGCCGGCCTCTAAGACCAAAGTGGAGTGGAACAGCTGGACTT |
| | | | | ACGTTTCAGGTATCTTTGCATAATCTGTTTTGTATTTCTTTCTTGATTT |
| | | | | CTCATTGCTAAATTTGATCGATCAACTTGTGGGTGACATACGTGAGT |
| | | | | TATATAATTACCATTAATGTTATTGCAGCGTGCTGGTGCTCATGATG |
| | | | | AGAAAGGGGATCCTATAATAAAAGATTTTTACAGCAGCCCTCTTACT |
| | | | | GCGAGGTATATGAAATTGCTGGCAGCAACCTATTAGCTAAAACTTT |
| | | | | GAGACTGCTGCTTCTGTAAATTTTCAAGTCCATAGCTATTTATTCCTG |
| | | | | CATCTAGTTTCTTGCTTTGTTTAATCAATACCTTGTTTTCAGTGGGAA |
| | | | | TTCTCATGATAATATGTTGCTTGCCAAATGTGAGTGTGGCTACACTG |
| | | | | GTGATTTAGGTTCTTCAATGGTTAGTCCTCTGATCTCTAGAAACAGA |
| | | | | TGTTCATTATACTCTTTTTGCTCACATATTTTTATATGCTCTTTTTGCT |
| | | | | CACATAATTTCATTATGTTCTTTTTGCTCATATATTTTCATTATGCAGT |
| | | | | TGGTCTTCAGTATGGAACAAGGTCTTCCCATACTGCCAGAGTGGCTT |
| | | | | TGCTTTAATAGAGTGAATGCCCGTGCTAGGACAGGGATGGATGTTG |
| | | | | GGCCAGCTAATCTCCTTTTAAGGTTGGAGTTGGTTGTTTAAAGTTTC |
| | | | | CTATTATGGTTATGTAGAGCCTAGTTCAGAGTGTATGTGGAAATTTA |
| | | | | GTGTATCCTTATTCTCAACTTGTTTTTGGTGGTTCTTGACATTATTAC |
| | | | | ATTGTTACCTTTGATTCAGTTTGTCTGGTGGGAATGTGGTGGGTAAA |
| | | | | TTCTCTCCTCATGAAGCATTTGCCATTGGCGGCACAAATAGTGTGAG |
| | | | | AGGATATGAAGAGGGTGCCGTTGGCTCTGGTTGCTCCTATGTGGTT |
| | | | | GGCTGTGGGGAATTATCCTTCCCTCTGGTACTCATGATTTAAAAGCT |
| | | | | CATTGCAATTTTTTCTAACTTATTTCACTTCTTTAGTTCTCTTTAGTAT |
| | | | | ATATATATATGCTACAATTTTTCTGCTGCTATATCTTTGAATATAAAT |
| | | | | TGTTTTGCTTTTGCTTGGGATGTGGCAATGAGGACTGACAATATTGT |
| | | | | CTTTCAGAAGGGATTTGATCTTGGGAACATTTTTCATTTTATATTTTA |
| | | | | ATTTATCTTGAAAAACATACAAATGGCTCATCTAATGTAGAACCTAG |
| | | | | CAAGCTCCACTGCTTATACTACTGTGGCTAAGGCTGTCAGTTAAAGC |
| | | | | TTCAGAATGCTTGGAAAATTATTACCGTAAAAATAATAATAAAAAA |
| | | | | GGTTCTGGAAATGTGTGTATGAAGTATTAAGAGAAGCAACTCTAAA |
| | | | | AAGTCTATACTGTAGTAAAGTGAATCAATGTAAGAGAAAGTACAAG |
| | | | | TGTGTGTAATAATCAAATAACATTGTGATAATGTTAAATTGATTAAG |
| | | | | CAAATTGATATGTTGTGATTTGTTGTAGTAGTGTGCAAATATCGTTT |
| | | | | GATTTAAGCTGATTGTTGGACATATATACTTTTGTTTTTTGTTTTTTG |
| | | | | TTTCAAAAGGCTCTCCCCATTGATTTAAGCTTACAAATTTATTTATTT |
| | | | | TTTCCAGTTTGGTCCAGTTGATGGTGCTCTTTTTGCTGATTATGGAA |
| | | | | CTGACCTCGGATCAGGCTCTTCTGTTCCTGGTATGTGTAGCCCACCA |
| | | | | ACCCAACCCAAAATAAAAATAAAAATAGAATTCAAATATAATAAAA |
| | | | | CGGACATGCAAAATGAGTCGAGCTTTGTTATTATCATTAGTAGCCCT |
| | | | | AGATATTTTTGGCATTGTTAATCATGCTAGTACCAAAGTTTGAACAT |
| | | | | TCCAGCTTTCTAATAAATGGATTGATTGGATTCACAAGAATGAAGCT |
| | | | | CGTTATTCTGTAGTTTAATTTGTAATTCTTTAAAACATTGTGTTAAAA |
| | | | | ATGGAAATTAAGAGATTATGAGGATCATTATACATTGATATATTTCA |
| | | | | GACATTGTGTGAACTGATTCAGTTGAATGACGAAGATAATGATAAT |
| | | | | GAATTGATTACATCAAAATTACACTGGGATAGTAACAAATTTTTTTA |
| | | | | ATTTTTAGTTTTTAATTTTTAATTTTTTTTGTGCTAAAAAAAACAACCT |
| | | | | GCAAGGCTGCAACACTAAAAGTATTTAACTTATTTCTTTCTCTATTAT |
| | | | | CTCCACAGCTTCACTAAAATTCCAGATTCTTCTCAAGTGTTCAATAAC |
| | | | | AATTTTTTTTTCATATTCTAAACTACCTATCCCATAATATCGTTTCTTA |
| | | | | GCTGACAATTAGGTATTGTGGAAGTATGAAGTCTTTGCCTCTTTTTT |
| | | | | CCGCATCTGAATAGACTGTATTTTTGACACAAATCTGTCCGGTTCTT |
| | | | | AAAAAGAAAAAATTGTGGATTTATCACTTAGTTGTGTGCGAGTTTTC |
| | | | | ATTACAAATTTTTTTTACAGTTAGAGATAAATAAGATTTTGTGATCTT |
| | | | | TATTATACAAGTGGAGGGGGGAGGGAGATGGTATCCCTCTTCAAA |
| | | | | AAACTCAAAATACCCTTAATGAATTAAATGCATATTTAAAAAGTTAA |
| | | | | TTTTGTTTAATTTTTCACTTGCCAATTAAAAGTAGTGATCGTATTTTA |
| | | | | TTGAACAAGTTAAATATGAAATTTTATTTTAAAATTTTCAATCAAATA |
| | | | | TTAAAGTAATGAGAATGATTTTCTTTTCATATTTGACATATGAAACAT |
| | | | | GCTATTACTGACCTGTGTATATGAAACTCGATCTAATAAGAAATTAT |
| | | | | ATGAAAATTATATTCTAAAACTTAAATGTTAATTTTTTATTATTCAGT |
| | | | | ATTCTCTATCATATAGATAAAACTTTTTCACTTTATTTTAGTCAACGA |
| | | | | TTGAACCTAATACACAAATTCCTTTTCACTTTATATGAACACATGATT |
| | | | | TTTGTTACTTTAAAGAAAACAAATAAACATTGTTTTAAGAAAACTAG |
| | | | | CAAAAAAATACAAATAAATGAATATGTATGTTCAATTGTTCAAACCA |
| | | | | AATTTTCTACAGCTAACTTATTTTAAGAAATAACATTAATATGATACA |
| | | | | TGCACACCCTTAATTGAATGTGACCTTTCATGTATCAATGAAATAAC |
| | | | | AAATAATTCATATGATATGGACATATAGTTAGATTGTTAATGCAGAG |
| | | | | CTTATGTGAGAGATGGGTATGATTATTAAATATTTTTCTTTTAAATTA |
| | | | | TTTCATAGGCGATCCAGCTGGTGCAAGATTAAAACCCGGTAGTGGA |
| | | | | TACGGGTATGGATTTGGTATCCGCGTGGAGTCACCTTTAGGTCCATT |
| | | | | GCGGCTTGAATATGCATTTAATGACAAGCAAGCAAGGAGGTTTCAT |
| | | | | TTTGGTGTTGGTCATCGGAACTAACAGAGCTTCAATTGTTTGGGTA |
| | | | | GCTGCTTGCATCATCTATCTGCGATTTTGTTAGAGTAGGAAAGGAA |
| | | | | AAATGCTGCTTGTATTTTTGCAGTAATTGGTCTGGAATCGATGAGAA |
| | | | | TGGAGGGCTACACAAACTTGAAAATATTTATTCTTTGTTTCACAGGC |
| | | | | TCAAGTACATCGTCTCATCTGTTTTTTCTCAGCTGTGAATGTATAATA |
| | | | | AGTTTAACCTGCGAAAATTGCCATTAAATCACTTGCTCAATTATTAA |
| | | | | CCTTGATTCTGCTACATGACTTTCGGCGAATCTTGCCATCTACAAACT |
| | | | | GTACGTTGAATCTTTAATAGAAATAATCGCGGCCAAATCTTGCCCGT |
| | | | | CTATTATGAGTAACTGGCGCAAATTCTACTATGGTCCCGGGATCATG |
| | | | | GTCCTCTTTATGGTCCCGGGCAAATGTTCATCATGTAAAACTCAAAT |
| | | | | GTCAATGTTCATATTTGAAAATG |
| 1776 | Kochia | gDNA | 7214 | TAAGTGGAAGAGATGAAATGTCTAGTTATGGAATTATGGTTGATGA |
| | scoparia | Contig | | TTTTGGTTTCTGGTTTGGTAAACGGGGAATTCTCTGGCTAGTTTTGC |
| | | | | ATTGATGAACAAAGTCATTGCACCAAGCCACCAACTGTTTTCATTTG |
| | | | | AAGGAGGCTCCCCGACTCATGTATTTTTCAGCCATGAATCTCGATCA |
| | | | | AGTGATTAATTTGATCTTAGGTCATTCATATTGCTCTTGTTGTTCGCA |
| | | | | TGGTTTTTTTAAAACTTTCTAGCATTCAGACTATGGTAATTTCTTTTTT |
| | | | | CATGCCAACACTTCCATCATTTGTCATAACCTATTGCTTGACGAAGA |
| | | | | TTCTGTGTAATGACATGTGCTTGAGTTTCCACTTATTCACAACAACCA |
| | | | | AGTTGTGAGAAGTTGATGATTTATAGGGCGTGTTTGGCAATAGTAG |
| | | | | CTGGCAGTGGGCGTTTAGTTGTGGAAATAGTTGGTAGTGCTAACAT |
| | | | | TGGTAGTTAGCGGTAGTAGTAGATGTTAAGTGTCAACAAGGGATTA |
| | | | | TACAATCTAATAATTTGGCAATGGTAACAGTTGTATATATAATAAAA |
| | | | | GAAAAATGGTATAAGAAGAAAAAATTGTACCCGCTACATGACAAAT |
| | | | | GTTAGTCCTAGTGTTGAGCAATATTCTCTTTAAGTACTTACCTCTATC |
| | | | | CGAAACATTATTTTGCCAAACCATAATTTAAATTACCAAGTAGTGTT |
| | | | | TTGATCTAGCCATACCACTAAATGCTATCTTATACTCCTACGCCGATC |
| | | | | ACGCCCATAGCATAGGTGAAACTATATAAAAAAAAATGCTAATGAA |
| | | | | ATGCCCCTATCCCAAAACAAAATAAATGCATTGTAACCCAAGTATTA |
| | | | | GTAGATTTGATCATGGTATTTGGTTGATTTTGATTAATGGCTGTAGT |
| | | | | TCCCAAGTACAATAGGAATCTTCTTTGGTGAATGTACCCACGACCCT |
| | | | | GAAAAACCTTGAATTTTGCATTTATTGTAATCAATCAATCAATCAAA |
| | | | | GCCTTATTATCATATGTGGGGTCGAACATGAACCAAAAGTGATTATT |
| | | | | CCAGGAGGTCATCCATATAGATTTTCCTCCTCTATTCTCTACGATCGA |
| | | | | GAGCCTCACTCTTGCAAATCCAACTGTTTCAAATCCTTTCTAACTCCT |
| | | | | CCACTTTATGGTAATCATTGTATAAAAAAACAAAAAACAAAAAACA |
| | | | | AAGAAAGAAAAAGAAAGAAAGAAAAGAAAGGAGATCAAAGGGCA |
| | | | | TAATAGTTAATTATGATGCCAAGTAAAAAATTAGTCTGGGTCTTGG |
| | | | | GAAATAGTGATGTGACAATGAAGAGGTTAACGGATGATGGTTTATT |
| | | | | CCACTGGAATTTGTTTTAATTGTTCGTCTTTCAAACCTTGTTTGAGTT |
| | | | | ATTTAGTTGCCTTTTGGTGTTGTTCAAATCAATTTATTATCGTGAATT |
| | | | | TGTGATGACTGAGATAGGTGTTTTCACAACGTATTTGGTCTAGAACT |
| | | | | AGTCACAACTCGCATTTTGTTTGTGGTATGTGTAGTAGTGACGGTGT |
| | | | | TTCCACGGGAGAAACAGAGTTATGCTGCGTTTGCATGCATTATTGTT |
| | | | | TTTGGTATCATCTTTGTTTCTGATTCAATGTTTGGAATTGAAATGGA |
| | | | | GATTACTTCCTAGTTGTTTAAATATCTTATTTGTGATTCAGGAAGTTG |
| | | | | AGTTTGGTTTAAAATGTATGTATCTTTGGATTTTAGAAAAAGCCAAG |
| | | | | GGAGGTCAAATGCCCAAATGTGACTTGTGATGAAGGGAATTAAAA |
| | | | | GAGATATGGAGAAAGTAGGATCATAGAGCTCTGGATAGAGGTGAA |
| | | | | TGGAGGAAGAGGTCCTTATTGATGATTGTAACATATGAATTCTTGAT |
| | | | | TCATGCAACCGTTATTCGTTTGACAATATCAAAGCTTTGTTATAATTC |
| | | | | TTAAGGCTGTAGTGTTTCCTTGTAGAAATGCTGTAATCTGCAAATTT |
| | | | | TGCACATTTAATGAATTATATGATGGGTTATCTTTAAATTGTCTATAA |
| | | | | TCTTAATTGTGGTTGTTTAAAATATGTCTAAGGGCAAGACGACATAC |
| | | | | CATTTCAGTATTTGTTGGGGATGAGAGTGGAATGATAAATCGAATT |
| | | | | GCTGGGGTCTTCGCTAGAAGGGGTTATAATATTGAATCTCTTGCTGT |
| | | | | TGGATTGAACAAGGACAAAGCTCTCTTTACAATTGTTGTCTCTGGAA |
| | | | | CTGATAAGGTTTTGCAGCAAGTGATGGAACAACTTCAGAAGCTTGT |
| | | | | AAATGTTCTGAAGGTTGTCTTTTGCTTTAGATGAAAATTAATTTTGC |
| | | | | ATTGATGTTTTCCACGAAGTATTATCTGAGATCTATTCTCATGGCCTT |
| | | | | TTATTTATGTTTAGGTTGTAGATTTATCCAAGGAGCCTCAGGTAGAA |
| | | | | CGTGAATTAATGCTTGTTAAAGTTGGAGCTGATCAGAGTAAACGTG |
| | | | | CTGAGGTATCATTCACAATATGTTTCGGTTCCCCAAAAGTGGAAGCT |
| | | | | AGTAATATTATTCTTGCTTGTCAGAAAATTTCGTTTAGTTGTAGCTAA |
| | | | | TTATTTTGAAACAGTAAATTTGTTGTATGCTAATCTTCCGCCATCATT |
| | | | | CATCCTGATTGAACTTTACTGTACAAGTATCTTTAAATCTTTGAGCTG |
| | | | | AGTGAATTCTAATTTATAATGTTTGCTACTTATATAATAACTTTTGTT |
| | | | | ATTTCCTTTTGATACTGCAGTTAATGTGGTTGGTGGACATCTTCCGT |
| | | | | GCTAAAATTGTGGATATTTCAGAAGAAAATCTTACGATGGAGGTAT |
| | | | | CACTGGAAACATTATGCAGATTAAGACAAATTTCTAAAGAACTATGT |
| | | | | AATCATCCTAACGAGGATTGACTTCCAAGAATTAAACACATGTTATC |
| | | | | TGCTTTATTTTTCAGTAGTCTTTGATGCTTTAGGTGCATTTGAATAAT |
| | | | | TTCTGTTTTAATATATATTCTAATAATGTTATAGTTACTGTATGTGAT |
| | | | | TCTATGATCCAGGAGATGTAACTTATAAGTATTTTGTTTTGCTTGGT |
| | | | | GCATACCCATTTACTTCCTGGAAATATTTCATGTAGCTTGTGTCATTT |
| | | | | ATATACTTGATCAAACAAGGTCCGAAAGGAAGTCATTTGAGCAACA |
| | | | | ACTTTTAACAACCAAGGTCCCTTTTTTTTTTTTTTGAATAAAAAACAC |
| | | | | CACATTGGCCACTATATCAAAGATTTCATGTAGCCTGCCATATCTAC |
| | | | | ATGTAAAGAGTCAACTCTGCACGCAATGCTTTTTGTTCAATGTTTGT |
| | | | | GCCTTTGTGGTGTCATGGACCTCATGGTGTGGACTGTGGAGGAACA |
| | | | | AAAACTACATTTGGTGCCTACTTTTATGACTCAGGATTATGTTATCC |
| | | | | GTAATTTGCTAATATGAAAACAATGAGTTCATGGTTATTGTTTTAGC |
| | | | | TCTTTGCAACCTGAATTATAAGTTGTTTTCTACTAGCATTTGATGTTT |
| | | | | AATGTTATCATTTACATAGCAAGTTGAATATTTATAGGTCACTGGAG |
| | | | | ATCCTGGAAAGATAATTGCTGTGCTAAGAAATCTAAGCAAGTTTGG |
| | | | | AATTAGAGAAATTGCTCGTACTGGAAAGGTTTGTTTTAAGTTGTTCA |
| | | | | GTTTCTGCATTGCATGTCGTATTTCATTGAAGAGTTGGAAACTTTTC |
| | | | | ATGTATTTCTGTATATGATTTGAAATGTTGATATTATCCTTCACAGAT |
| | | | | CGCTCTAAGAAGGGAAAAGTTGGGTGAAACTGCTCCTTTCTGGCGT |
| | | | | TTTTCTGCAGCTTCTTATCCTGATCTTGGAGAAGCTATGTCTGAGGA |
| | | | | TGCCCTCCCTGAAATTTCAAGACGAGCTCCTGAGACTGAATCATCAG |
| | | | | ATTTATCTGTTGAAGTATGTTCTTTGATAGCTCTCCTTATTTTTGGCA |
| | | | | ACTAATTTATGTTTGTGTATTTACTTTATGCTTTCTTTTGATGAAGTCT |
| | | | | ATAAAGAACTTGACCTTCTGCATAGTTAGAAACTATGTTATTTAGAC |
| | | | | TCTTCATTTTACCTGAAGTACCCGTGTTGGATCCTTGAATCTCGGAC |
| | | | | ATGGGTAGACTCTCGGACATGGGTACGATACTTGGACACTTCATTTT |
| | | | | GAGCTAAAATCTTGGTATTTTTCCAAAAATTAGCCTAGTTGGACACT |
| | | | | TGGAAACGTACCCGTGTCGGACATAGGTATACATGTCCAAGTAACA |
| | | | | CATGTTAGAAATCTTTGGAGCTTGTTTCACAAGCTGTTAAGATTGCT |
| | | | | CAAGGTGTCAAGGCCTCATCTCACTTATTAATACAGAGTTGTTTTGT |
| | | | | TAGAAGTCTCAGAAACTTATAAACCAGGCTGAAATTTGAAAAGATA |
| | | | | GGCTAGTATAATACTTTGCTGTTTTTCTCTCAAAAAAGTTCTAAAAC |
| | | | | GACAAAGAGGGCTAGTAATATTAGAGAATTACTGATTTAGGACAAG |
| | | | | GGCTAATGAATGAGTCTTGATAAGTTCAGATAAGGGTCAATAAGTT |
| | | | | TAAATGAGGTTAGATAAGGGCTGAGAAGTTCATATTATGATAATAG |
| | | | | TTCAAATAATGGATTAGTGAAGGAACAAAGGTGAAAGATAGTGATT |
| | | | | TAAGTTCCTAGGGATGCACAATTACCATGATTATTGATACAACAACA |
| | | | | ATGCACGATTCTATAGCTGAAAGGGGATCTTTATGCTGCAATTCATC |
| | | | | TTCCTTGGGTCATCTTTTCCATTAAAGAGAATAATTTCCTGGTGAGA |
| | | | | AGAGAAATCACGAGAGGAAGTATGAAACTTCCTTATAGGACATAGA |
| | | | | ACAGATTGGACACTCCAGGAATGAAAAAATAATGCTCTAATGAACT |
| | | | | TGCATATCACCATATTTGTTGAGGCAAACTTAACTCCTGACTATATA |
| | | | | ATCATATAGTCAGACTGTTTTAGGATTCTAAACAATTGCAGATAATT |
| | | | | GTGTTCATGCTTGGTCGAAAAGTTTTTCTGCATTATATTTGAAACAA |
| | | | | ATTATGTCAGCAGGTGATAAATAAATTGTGCCATGCTACAGAAAGC |
| | | | | GAAAAAATTCATACATTGATTTATTTAAGGTAATTCTTCAGATTGTA |
| | | | | ATGTGTACTTCTACAGATTATGGAGCCTCCCACAATTGTTGTGAAGA |
| | | | | TGTCCACTGGTTTAGGATTGTTATGTCTAATTTTTTTTCCATGACCTA |
| | | | | ATTTATCACTTATTTTCGAAAATTGAACTGCTATTTCCACTATAATTC |
| | | | | GCTGCTTCTCCACTAATTCATATAACTTCTCCACTTAACTTCTCCTTAA |
| | | | | ACTACTTAATGAACTACTTTCCCCCTTCATGAAGATGTCTTCCAAGAA |
| | | | | CATGGAGTCCATGCGAGTCGTTTGCTGGCCATGGATTTGGGGACAT |
| | | | | CTTTTTGTCACGAATTTTGTATTGTATCTCTGTGTTATTGGGAATATG |
| | | | | CTACAAAGACACTATTTAGTGTTTGCCTAATTATATTGACTCAATGA |
| | | | | GATATACTTCTATAGTTATATACTTATATACTAATCAATGCCCATTCA |
| | | | | TGGGTTAAAATTTTGATATTACAGGGTGATGTTTACCCAGTGGAACC |
| | | | | TTTTGATGGTTTTGCAGTTCCACAAGTCCTTGATGCTCACTGGGGTG |
| | | | | TCTTGAACGTTGAAGATGTAAGTGACCCTCTTGTTTTCTTCAATTAAA |
| | | | | TTATTCTACATTTCTATTGTATGCTTTCAGGGTTTCTGTTCATTTTTTT |
| | | | | AATTAATTCTGTACTTAGGTCTCATTACTCTCTTAGTCTCTTGAGCTC |
| | | | | TTAATTTACAGCTGTAATGGCCCCCTCTTTGTTTCATTGCCAACTATG |
| | | | | TTGGCAGATGATGTGGGATAAGGCAATGATATTCTGAATTTAATTG |
| | | | | AATGTTGAGTAAAATGGATGTGTAGTTGGCAAGAAGCATTTGTTTT |
| | | | | GGTTGGGGTTAAGGTGTTTGGGCGGAAATTTAGTAACGCTAGCATG |
| | | | | AATATATTAAAAACCATTAAAATTTCGTATACTTGTATGAACTATGA |
| | | | | AGTATTTCCTTATCTCTGAAATTTCTCTGATAGCTGTACTAGCTATAT |
| | | | | ACAATGTCTACAAGTTTTAATATGGTTGAATTATTAAATTCATAAAG |
| | | | | ATGCTTAGCACAAAAATTTTATTTAGTGGTTTTTCCTATTCTAAACCA |
| | | | | GATAACTAAGTTCAGACTGCAATCAAGCTTAGCAGTAGCACAAAAT |
| | | | | GCTAGAAAAGCAAAACTTATTATAATTTCTTTTCTGTCGAGACTTTTT |
| | | | | TACAGGCTTATCTAGTTATTTGTGATGTTTGGTATGTCCACTCTATAA |
| | | | | TTTGATAGAATGATAGAGTAACTGTTTGCTATGTCCACGAGGTAACC |
| | | | | AATTGTAAAAATTTAAATGTATATGAGGACACTGGAGCAACCTAAT |
| | | | | GTTTGGACATATGTATGCAGACAAGTGGGATGCGATCACACACTCT |
| | | | | TTCGCTGCTTGTTAACGACAAACCTGGGGTCCTTAATGTTGTTACAG |
| | | | | GGGTTTTTGCTCGAAGGGGTTATAATATTCAGGTGTGAGTCAATTCT |
| | | | | TACTCTGCATGATACAATTTAAGTTCTGCTGTCACTAGCTAAGTTTAT |
| | | | | ATTGCTGATTTTTCTGGTTTTCAGAGTTTAGCTGTGGGCCATGCAGA |
| | | | | AGGGGAGGGTCTATCTCGGATTACTACTGTTGTACCTGGTACAGAT |
| | | | | GAATCAATTAGCAAATTGGTGCAACAAATTTATAAGCTAGTTGATAT |
| | | | | TCATGAGGTGAGAATATCTATTCATTTGATCGTTGTTACCCACAATA |
| | | | | TTTCTATTTTTGACTCATACTCATAATTTATTATATTATGCTATTTTAT |
| | | | | TTCTGACTTTAGGAAGCTGCAAACATGGAAAGAATTTTGTATGAGTT |
| | | | | TTGACGTCAGTATTTTAGTTGTAACGAATGTCTTTGGGTGTTTCTAG |
| | | | | CATGTTTGAAAGCTAATGTTATTTGTTTAAAATTTAATCTATTTATAT |
| | | | | TTTTATCCAGGTTAGAGATATCACCCATCTGCCATTTGCTGAGCGAG |
| | | | | AGTTGATGTTGGTGAAGGTTGCCGTGAATACTGCTGCACGCCGTGA |
| | | | | GGTCCTTGACATTGCTGGCATTTTTAGAGCTAAAGCTGTCGATGTAT |
| | | | | CTGATCATACCATAACACTTGAGGTAATAATCAATTCGGAAGGTTGC |
| | | | | TCAAAAATATTGTTGACTCCGTTTCAAATGCAACTTTTTTAATTTGAT |
| | | | | GTTTTGATATAATTGCTACCCTCTTGTTTATAGCATGTTTTAAAAATC |
| | | | | AAAGTTTGGGCAATTTTCTTAACTATCTTAACTACAGTACTACCTCTG |
| | | | | TGTTTTTTAGATGTCCCACTTTGGGTTTTGTGATTTTTAAGGTAACAT |
| | | | | TTTGGTGTAGGTGATTAGGTGAGTAGATGGGTAATTTAATAAAGTA |
| | | | | TGTGGGACCCATAGA |
| 1777 | Kochia | gDNA | 5508 | TTTTTTTTTTGACGCGGAAGGATCATTTTATTCAAATATAAGCCTTCT |
| | scoparia | Contig | | TAGTATCTACATCATCAATAGAAAAGAACTCAAAAAGACTTAAAAA |
| | | | | ACAGAAACGGAACTATAACCACAACACCTAAACTTTAATCTAATCAA |
| | | | | GCTCAACTCCACGAAAGTGCATACAATCATGTAAACATGATAAAAT |
| | | | | ATCGACGTCAAAGCATATTTCAATAGAATCTACACCACCGGTGCTGT |
| | | | | AGATGTTATTGCAAAAGCAATCGCCCCGTAGATCAAACCCTTTAATT |
| | | | | TCATTCATTAGTATCCTTATTTGGTCATACTCTTGCATTTTATTTAGAT |
| | | | | GATATTCAAAAGGAGCAATGCATTGGGAAATTCTCCCACCTTAACA |
| | | | | ATGAGGGATAAAAAAAGCACCTATTAGGTGACTTTTGAAGGCTCAT |
| | | | | GCACTATAGAATTTGCGTCTTTGTTTCATTTATTCTTTGGTGTATAAG |
| | | | | TGAAGTTTATTGCTTACTTGATGGAAGGTTATCTCATGTCTATAATA |
| | | | | ATTTTTGAGAATTCTAGGTTAAGGTATGAGAGAGGAGTAAACTTCA |
| | | | | ATTTCCTGACTCTTTGTAGTTTGTAGAAGTCAATTCCCGATGTATCCC |
| | | | | TTCTGTTGTTTTGCTCCTCAGTCTAGAACCCTGTGTCACCTCCTTTCT |
| | | | | GAATTGGGGTTTTAAAGAGAGTTACTGTATATAGTTTGTCAAGTTAC |
| | | | | ATTGCCCCAAATCCTTGAACGGAAGTCAGTTTTCCTAAAGAATGTGG |
| | | | | TTCTCTCATTGAGTCTTGCCATGTTTTTCCTATGTATTTAAATCTCTGT |
| | | | | TTGGGTAAAAATGAAATGGCTATGCAAGATGTTGAGGTTTATATAT |
| | | | | TTTTATATTTCATATCCAGGGTGATGCGCCACACGATTTCAGTTTTTG |
| | | | | TCGGTGATGAAAGTGGGATAATCAATAGGATTGCTGGTGTTATTTC |
| | | | | TAGAAGAGGGTACAACATAGAGTCTCTCGCTGTCGGATTAAACAGG |
| | | | | GATAAAGCTCTTTTCACTATAGTAGTGTGTGGTACTGAAAAGGTGTT |
| | | | | GCGCCAAGTTATGGAACAACTTAACAAGCTTGTTAATGTTTTGAAG |
| | | | | GTAATGATCTAATTTAAACTTGTATTTAAGTCATCTCTGATGCCCTGA |
| | | | | CAACGCTGACGTTGGTGTCATAAAGATCTTTAGATTGTACACGGCTT |
| | | | | ACAACTTTCACCATTTGGCGCGTAGTAAAGAAACCTGAATTGCCCAT |
| | | | | CAGATCTTCTGATCAATTGAATTTTGAAAATAGATATATAATGGTGT |
| | | | | ATTATAATTGTGGTTATCCTATCCATGTATGTTTGTACATGGTAGTG |
| | | | | ATGGTACATTGTCTTCACAGTAATGCTGGAGAGCGTAGGGCTTGTG |
| | | | | TTGCTACCTATTGTTTAATTACTCCCGTCCGTGTAGGTTGAAGATCTT |
| | | | | TCAAGAGAGCCACAAGTGGAACGTGAATTGATGCTTGTAAAACTGA |
| | | | | AAGCAGATGCAAATTACCGTGCTGAGGTATTTCAACAGTTTTGGTTA |
| | | | | ATTCTCATAATGGCATAATTATCACTTTAAATGTTTTTTCGTATTCAA |
| | | | | TTGTCAGCTGGTAATGAAGAAGAAATTGCGGTTAATCATAAGAAAA |
| | | | | ATGTTCACAGAAAGCTCAAGTTCTTCGGAAACTTTATTGCATAAAAG |
| | | | | TATTGTTTAGAATATGTCTTAATTCCAATTATTGCGTAAGAAGTCTCA |
| | | | | AAAATCACCAATTACCTCACAAAACTCACCCTTTCAATAATTAACTAC |
| | | | | CTATAATAGTCACTCCTGTTTTACTGTACTCCCCTTATGCAGTTATAT |
| | | | | CCATCTTTTAGTATGTCCGTCTCTCTCCATCTCCATGAATTATATGAT |
| | | | | CCGGGATTTAGTTGCACGTTATCTTCAGTTATTATTTGTAATTTGCTA |
| | | | | TTATGCAATCACAAATGAAGGTTTCAGAATCTTTTTCACTTGAACAT |
| | | | | GAAGTCAACTTGTGAATTGTTTTTAAGTATCTGTTATTACTAAGGAA |
| | | | | AGTCACGGGCATTAGTAAACTCACATTCCTCTATTGTGCAAATTTTC |
| | | | | ATGAAACATAGCCATCAATATTCGGTATTGGAGTGGTTTCCCATATT |
| | | | | GTATACACAAATTTTCACATGATTGTGTGACACTCAATTATTAACTCT |
| | | | | CTATTTGCATGGCTTTTTAAAAATATATATGGATGGGCGGAGAGGA |
| | | | | ATCTATGTTAGTAGTTATCTGCTCTGTGCATTGGTCAGTTAAGGATG |
| | | | | CTGAATTACCCAAATGTTCTATTTTTTTGGATCTGCAGATAATGTGGT |
| | | | | TAGTGGACATTTTCAGGGCAAAGATTGTGGATGTGTCAGAAAATTT |
| | | | | AGTCACAGTTGAGGTGAGTGATTGATGAATGGTTATTTTGTGTCCT |
| | | | | AAACTGATGCAATTTACTGTGCTAATGTTGTCTTCATGTAAATCATAT |
| | | | | CCAATTATATATAGCTCGATGTGTAAGCTCTAGATTTTCATATTGCA |
| | | | | GCCTACTCTATAAATTGCACCTTTTATAATAATGTGGTTAGTAAGAC |
| | | | | TAAATTCTCCTTTTTATTGTAGACTTAATAGTTTTACAGGCCTTTTATC |
| | | | | TGGTGCTGTGCAGGTGACTGGGGATCCTGGAAAGTTGGAAGCTGT |
| | | | | CCTGAGAAACTTTCACAAGTTTGGAATTAAAGAAATTGCAAGGACA |
| | | | | GGAAAGGTTAGTCTTTTTGTCACTTGAATAGAAGAATCAGATAGAT |
| | | | | GGAAAGTATACAAGTCATAGTACTGAGAAGGAAAAGTATAGTTGG |
| | | | | AGTAAATAGCTTTATAGTCTACTGTTGATTTTGTAAACGAATTTTTTC |
| | | | | AATGCTAATTCTCAGTGAGACCAATGAAGATAAAGGTTTTTGATGTT |
| | | | | TTGAATGTGTGACAGATTGCTTTAAGACGGGAGAGGATAGGTGAG |
| | | | | ACAGCTCCATTCTGGAGATTCTCTGCTGCTTCTTATCCAGATCTAGA |
| | | | | AGAGAAGGCCTCTATTGCTGTTGTTAAGTCTGCAGAAAGAAGTATG |
| | | | | AATGGTAATGCTGGCTCATCGTTGAGTGTAAGTAAAGTTTCTCCTAC |
| | | | | CGAAAAATCATCTCATTCCATTCTCCCCATTTGTCTCTTCTATGAGCG |
| | | | | TCTTTTCTTTGTCGCTTTAACTTTTAAAGAACATATGGACTTTTATAT |
| | | | | GCTTATGATTTGCAATCCTGCTTGATTTTATTTCATATTTTCTATCTAT |
| | | | | AACTTGTGATTGCTTGTCATGAATTCAATCATAAGCTTTGTGTTTGAC |
| | | | | AATTTGTATCTAAAATGTTTCTTTTGTTCTCTACAGGGTGATGTTTAT |
| | | | | CCGGTGGAGCCTTATGATCTTTCTCCAGCACATCAAGTACTTGATGC |
| | | | | TCACTGGGGTGTCCTTTATGATGATGATGTAAGCTTTATTTGTTGCT |
| | | | | TCCCGAATTTTTCTTGTATCGTTGTCGTGGAATTTGCATAAATTGTGA |
| | | | | AATTCAAGCTTATACTCAGTTGTTTTCATCTGAAGGGTCAAGTTAAA |
| | | | | TGCCTTTGGCATGGTTTTTCCCCCTTTCTAGAGTTGGGTCGTTTTGCA |
| | | | | ATTTGATTTTATGATGAATTTACTCTGCTAGATTTTTTTATGTGGGAG |
| | | | | TGCTTTCCTTTTTCAATTTTCAGTCAAGCGGGCTTCGATCACATACTT |
| | | | | TGACCATGGTAGTTAATAATGTTCCTGGAGTTCTCAACACTGTAACC |
| | | | | ATGGTGATTGCTCGTAGGGGTTATAACATTCAGGTCAGTATGACTCT |
| | | | | CACTGTATGTATTAAAAATATGTATAATGCATTTTTTTTGTGATGTGG |
| | | | | TCTCATATTTCCTTTTCTTCCCCATCCTCAGAGTCTTGCTGTTGGCCCT |
| | | | | GCTGAGAAGGAGGGTCTTTCTCGTATCACCACTGTCGTTCCCGGAA |
| | | | | ATGATGAATCAATTGGAAAATTGGTTCAGCAACTGAACAAATTGGT |
| | | | | AGATCTATATGAGGTGAGATTATATGTGTAGTGGGCATGTTGCCCA |
| | | | | AGTAAGGTAGTTTTAATCCATGCTTATATATTTTTAGGAAAAATTAA |
| | | | | CAAAAATAATCCGACCTTTCAGTGGTCTTCCAAAAATAACCGGACCT |
| | | | | TTGAATAATCCCAAAATAATCCCACCTTTGCTCCCCATCTTCCAAAAA |
| | | | | TGGTCCCTTACCGGTTTGTGACCTGTTGACTAGGTTAATTTGCAACG |
| | | | | TGTTGCTTCTACATTGGCTGTAATTAAATTTAATCTAAATTAATCAAC |
| | | | | ATTTAATCATAATTAAATATAAAACTAATTATAATTAATTAAATTATA |
| | | | | ATTATAATACCATCCCTCACCACCTCCTGCCACCACGCACCACCTGCC |
| | | | | ACCACCACACATCACTTGCCACCATGACCCACCAACCACCACCTCAA |
| | | | | CCATGCACCATCTCTCTCCTCTCTCCTCCCTCACCACCACACCAACCA |
| | | | | CCATCGCCACACACTGAAACTACCTACTCAAACCCAGGCCACAACAA |
| | | | | CCATCACAATCACTATTCTCCCAAAATATGGCGCAAGGAATCAATAC |
| | | | | TTAACGCCGCCTTAACTGGCGCACTCTCGTTCAGAATGCTCTTTTCTT |
| | | | | CTCCAATCTCAATTGTTGCTCAATCTCCTCCTAATCAGTCATCTTCTTT |
| | | | | TGTCGAAGACTGTTGCAACAATGAGTTTCCATTGGTGGACGAAACA |
| | | | | TCATCATCATCGAGTAATTTGAGAACTAATGAGGGAATTGCAAAGG |
| | | | | AAGCATGGGAGAGGGGGGCTTAATTTTTCCATATTTTTATTGTAACA |
| | | | | ATTTTGACCCTACTATTTCATGGAAGAATCTCAATTTCTTATGGAAAC |
| | | | | CCAGTCTCATTGGTGCAATGCAGTAGTTCAATTTATTTTAGCTTTGCT |
| | | | | TTTGTGTGAAGTAGTGATTAGGAAAATCATTGGTGTTGTCATTTGTC |
| | | | | AGAGTTTATCGGTCGATCCTCCTCCTTGCAATACCTTAATTCTCAGTT |
| | | | | GAATTACCTGTTTTTGGTAACATTGTTTGGCATAGATACTTAATATTT |
| | | | | TTCGTATGACCTTCCATCCTTTGCCTTGGCCCATTGTTCCAAGGTCCG |
| | | | | GGTTGATGGCTCAACATCATGGTCAGCAGGCCTCATCAAATATTTTC |
| | | | | AAACATTTTATACTCCATAGTTATTTATTGATCATGATAGCCTGTGGT |
| | | | | CTGCACCTTCTGCCTTGTTCTGGTCTCCAAGAACCAGTCTAGGCGCA |
| | | | | GCACATGGACCATTCAGGAAGGTGCTGTGCTAGGTTGGGACATGA |
| | | | | AAATGCTGGCCCGCTTGCTAGCATGGCCAACTGCCTGCTTTAATTCC |
| | | | | TACTCTTCATCAAAATTGTGACATGCATATTGTTTCTAGAACCAAACT |
| | | | | GTGGTGCTACGTCTAAAGATCTGTCAATCTAACTATAAAAATGCTGT |
| | | | | GCCTGCAGATTCAGGACCTTACACACCTGCCATTTGCAGAGCGAGA |
| | | | | GCTTATGTTGATCAAAGTAGCTGTAAACACTTCTGCCAGGAGAGAT |
| | | | | GTCCTTGATATTGCCAATATATTCCGTGCAAAAGCAGTAGATGTTTC |
| | | | | TGATCACACCATAACCTTACAGGTATGCCATACACGAGATGTTGTGC |
| | | | | TTCACTCTTTTTGCACTTGCAATAATGAGGTGCATCTT |
| 1778 | Kochia | gDNA | 2741 | CATGTCTTGAAAGGGAAAAAAATCAAAGTGGGACAACTAATTCCAA |
| | scoparia | Contig | | ACTTCCTAAAATAGAAACTTGGACATCTAAAAAAAATGGAGGAAGT |
| | | | | ATATTTTTACTATTTTCTTATCTCTTGTAGCTAGCACTTTATTTTCTGT |
| | | | | CCTTATTCCTTGCTTGCCAAGTTTGAGTGTCGCCATATTTTTTGACCA |
| | | | | GAGAAAGTACAAACTATTCTTTGTCTGTCCCAATTTTCTATTTCTGAA |
| | | | | ACCTTTCTTGCATGTAAAGATTCTCATTCTACCTATTGAAGATAAATA |
| | | | | TTGTGGAAGGAATGATCCATGGAGAAAAACATGGCTTGAAGTTGA |
| | | | | CTAGATCTTCTGAAGATTTTTTTTTTAATACATTGATGAACTCATGCA |
| | | | | CTTGAATTTTCCTAAAATGGATTTAGCATCTCAATGAATAGCATTTG |
| | | | | CTTAATCATTCTGTTGATAGTCACGATACACTGTACAAAAGCTGATT |
| | | | | CTAATTGTGATGCTTCTGACCGACTGACTCCCAGCTTACCGGAGATG |
| | | | | TGCATAAGATGGTTGCTCTCCAAAGATTGCTGGAGCCCTATGGAAT |
| | | | | ATGCGAGGTAGGATTTCTGTACATTGTCTAAGTTGCCTAGTATGTCA |
| | | | | AAAATATCAGTTGTTTCTTGCTCGGTTCTCACATCATCTGTATTGACA |
| | | | | TTATTCAAATACCTGTAAAGGAGGATTTCACTGTAGATATGGTGGCT |
| | | | | TTGCACTTACTTTTCGACCAAAAATCCACTATCATGGAGACTACTCTT |
| | | | | TATTCAGTACATCAACAAACATTTAATATGAATGGATAATTGGATAC |
| | | | | TATGAGTGAGATGTACTCTTGAATATGAATGGATAGTATGAGATGT |
| | | | | AACGTTTAATATGAATGCTATACACTTGAATATCTGAGGATATCTGA |
| | | | | CTTGCCATACTGCCAGTTTGCTTCCTCATAGTAGTATTGCATCTTCTT |
| | | | | TTACACTACATAGCTTGTTACTGTATTGTTTTAAATGTCTTAATAACT |
| | | | | GCATACATGTTTTTGTAATAATATAGGTTGCGCGTACTGGGCGAGT |
| | | | | GGCCTTGGTTCGTGAGTCTGGCGTAGACTCGAAATATCTTCGTGGA |
| | | | | TACTCTTTTCCCGTATAAGTGTCTGAGCAAAAAAAATACTTCGGGAG |
| | | | | GAAGGCATTCGAGTTTTGTCAGAATATACTTTTGCAATATTTTCTTTA |
| | | | | TTGTTTTTATAGGTAGAAAGTAAGCAATCTTGTAACCTATTTTTGTTG |
| | | | | TTTGCTTTTGTAGAATTTTTTTCAAACCAGAAAACTTTTGAGAATTGA |
| | | | | ACAAATGAAGTGTATTTCTTTCTAAGGTATATGTTCTCTATCCATTGC |
| | | | | CGGTTGGTTTTCTTCCCATTTTTGTGTGTGTGTGTGTGTGTTTGTCGA |
| | | | | TACTTGTGAGCGGTACTATGGAGCTCGATACTTGTACGTTGTTCAGA |
| | | | | TGTTGAATAGTAATATACGAAGTACTCTGTCGTATCTTTATGTTTATC |
| | | | | CATATTTTTCTGCAGATTAAGTTTACTGTAAATCATAACCTTGTGCTC |
| | | | | ATGATGAGCGTCCAAATCAGATTTTCACATGCCAAAACTATATGATG |
| | | | | CACTTCTTTCACTAGTGTATCATATACAAGAATAAGTTTAATGACAA |
| | | | | CCCACCAAATTAAATTTTTTAAAATAAGTTTAGTGCGTCCTCAATAAT |
| | | | | ATTTAATTTTTATAATTTTTGAAAATAAAATGGACATTGACATATGTA |
| | | | | ACGAAGGGAATACTTTTGGGGTTTAATTCATTTAATTTAAAAAAAAG |
| | | | | TAAGTTGAGACTTGACAGCAGGACCAACAAATCGTGATGATGCGAA |
| | | | | ATTGTGAATGACACGATGTTTGTGCACTCTCCTCCGTCCTCAGTCCA |
| | | | | AAACTCAATCACTCAAATCTGAAATATTCACCAGAGCTTAACCACAG |
| | | | | CTTAATATCAGCACCAAGTCAGTCGTCACAGCTTCCGACCACCACGA |
| | | | | CGAGGTTCTTTTTGCTGCCTTTGAGCCTCTCATTTTTGTTTTTGTCAT |
| | | | | GCAACTTTATATTTCAATATTTATATTTATATTTTGAGGTGTCAATAT |
| | | | | TGATAGGATCATAAACAATTATACATAAGGTTTGAAGGCGAATCGA |
| | | | | ACAAGATTTCCTATTACTCCCAGACCCAGTGTAAAAAACTAAACAAG |
| | | | | TCAAAGTTTATGAGTGAGTCAATAGTATCAAAATATAGCAACAAAC |
| | | | | AAGATTCCAATGTTGAAGTTCATTTTGTAGAATTGTGGCTGAAATTT |
| | | | | CAATTAAACTAGTTACTTTGTTCAATTGTCAGTTAAAGTTTTGCCTAA |
| | | | | ACATTGCTTGTGCAGCTGTTTTTTTAGTTTTTGTTTTTTGCATCTTATT |
| | | | | GATGGTAGCTGTTTGATTATTCTGATTTTATTGTTCATACTTAATTAG |
| | | | | TATTACTGAATTTGATTTATGATTTTAATTGCTATTATTGTGTTTGATT |
| | | | | GTTTGATGTCTATTTTGAGTCTAACTGAATAAATTATAACTACGAAG |
| | | | | TATTATTTTTATGAATCAATTTATTATTTTTCATAGACAATTAGAAAA |
| | | | | ACAAATAATCAACGACCTATTCATCCTGCTAGCTTAGTACTTGGGAC |
| | | | | TGACAACTTTGATGAGAGAAAAGAAGAACACAGTTTAAATAGTTTT |
| | | | | AAAAATCAACATTAACTAAATTATAGATAGGGGTAAATGAGATTCG |
| | | | | AACGTCTAACGTACTTGTCAGGTTGGCTATGGTTGAGTTGAGATGG |
| | | | | TCTTTTAATATTATCATGTATATGACTCTACAATTTGTTATGCAGCGC |
| 1779 | Lolium | gDNA | 1203 | AACAGTATTATAAGGACTAGGGTGTTCCGTGTCTTATGCTGGATTAA |
| | multiflorum | Contig | | ATTTCTTGATGCTGATCTGGCAAATGCTGCAGGTCACTGGAGATCCT |
| | | | | GGAAAGATTGTTGCGGCACAAAGGAGCCTAAGCAAATTTGGGATC |
| | | | | GAAGAAATTTGTAGAACGGGAAAAGTATTTTTCTGGACCATTTGCT |
| | | | | ACGTACATGCATGGGCACATTTCTTCATTGTCTAATTCTTCTATCTCA |
| | | | | ATGTTCTTAGATTGCTTTGATTCGTGAAAAAATTGGAACAGCTGCCC |
| | | | | GTTTCTGGGGATTTTCTACTGCTTCTTACCCAGACCTCATAGAAGCA |
| | | | | TCGCCCAGAAACCCTCTTCTTACTTCTCCGAAAAAGACGGTTAATGG |
| | | | | CAGTTTTGATCAGCCATCCAGTGCTGGGGTATGTTTCCATGAATATA |
| | | | | AGACCAACTACATATTTATTGCATATTCCTTCTTGTTCTGCTTCAACT |
| | | | | GTTCTAAAAGCGAGGCAACACTTAGGCTATAGGTGCCACAGTCTGC |
| | | | | CTAGTACCTTTTAAAACGCTGTTACTCCCTCCGATCCAAAATACTTGT |
| | | | | CCAAAAATGGGAGAATCTACACACTAAAACACGTCTAGATACATGC |
| | | | | ATTTTTGGGCAAGTATTTTGGACCGGATAGAGTATTTGTAGTTTTAA |
| | | | | ATACTTGGTAATCTACACCACGCTGAAAAGATGCCCCTTTTACATGA |
| | | | | ATGACACAATCAATAATTTGATGTGCAATAACAGAGAAGATCTCAC |
| | | | | GAACTCTGCATATCTTTAATAGCTATAGTGTAAACAAAGGCAACTGA |
| | | | | ACTTCTCTGTCACCAGTAAACATCCCAGTGCAGCCGTAGCACTGTAA |
| | | | | GTGAATTTATAGATGAAGGTAATTTGTATACCTGGCCTTAGGAAAG |
| | | | | CAAAAAAAAAATTGCTCATACATCGAGGGGAGATACCTGCGTGCTG |
| | | | | ACAAAACTAAAAACCACACAGAGATACTCATCCCCACGCAGGCAGG |
| | | | | GATATAAAACAAATAACCAGATTAAGTCGAACATTCCTATCGTTTCC |
| | | | | ACTACATCAGACACAAACGCCCCTCAAAGGAGGCCAACATAAATAT |
| | | | | TTCAGCTTCCTAGCTTGAAGTTGAAGTCTGATCTGCATCTTTAACAT |
| | | | | GAAATCCTGGTATGTAGTCTTCAGCGACCCCAACATTGCTGCATAGA |
| | | | | AAATATAATTTGGTAAATGAGCGACACAGGATTCA |
| 1780 | Lolium | gDNA | 1074 | ACTGGCATGGTTATACTTCTTGAGAAAATATTATAAGTTAATTCTTCT |
| | multiflorum | Contig | | TTTATTTCTAACTAGATCTAATGAGCCTTATTAATGTAATAGATAGCT |
| | | | | TTGCGGCGTGAGAGAATGGGACAAGCGGCTCCCTTTTGGAGGTTCT |
| | | | | CTGTAGAATCCTATCCTGATCTTGAAGTGAAAAGGCCTTCAGAATCT |
| | | | | ACCCTAAGCACTCAATCAAAGACAACCAATGGCGACTCTGAAGAAT |
| | | | | CTTTGCAGGTAGGACATGGTAATTCTCATCATTCATATAATTTTTCAT |
| | | | | GTATCCATTTTTTCTTTAGCATTGCACCAACATTGTTTCCATTTATTTT |
| | | | | GTACCTGGATTGTTGTGTTGTTTGAATTCTTTTCATGCATTCAACAGA |
| | | | | AATCAACACGTGCAATAGAATACTCTCTCTTCCATTCTTCATTAGTAA |
| | | | | GGTTGTTCTTTTCTGATTTGGTCTTAATGGGGATTTATTGTTGTTCAT |
| | | | | TTTATTAAGATATGTCCTAAATATATTTCTTCCAATCTGAGTTTACAA |
| | | | | GGGAAAACAATATATAGACTAGATAATCTACCAATATTAAATCCTTG |
| | | | | CAAGTGTACCTTCTATGCAATGCGTATTGATGCGATTAGCTACATTT |
| | | | | GCCTATTTAATGCATATGTTTAGTAGACAAGCACAACTTGATGCGTC |
| | | | | ATTTAAATTGTTTGTATGATTCATAGTATCAGCATTCTGTTGACAATT |
| | | | | ACTTGTTTTATTTTAGAAGTTGTAAAAAGTTGGCTATATTATATAGCT |
| | | | | TAACTACATCTCACAAACAATGGTAGAAAATGACCATTACTAGTTTG |
| | | | | TTTTGTGTAGACAATTTTATTGCAGAAACTTTTTCTATTATCCACATA |
| | | | | GTTTTTTTTGCAGTTTGATGATTGATTTATATATTCTTATGTTCTTATT |
| | | | | ACTAGGGCGATGTTTATCCAGTGGAATCTTATGAAAGCTTCTCGATG |
| | | | | AATCAAATTCTTGATGCCCATTGGGGTGTGATGGCTGACACTGATG |
| | | | | TAAGTTCTTGCATTCTTCTTTCACAAGAGCTGCTGTGTTGCTGAAAG |
| | | | | TTAATGGCAAGCGTCCCAAAGTCTAGTGG |
| 1781 | Lolium | gDNA | 838 | AATGGTCGCATTACAAAGGATGCTTGAGCCGTACGGCATATGTGAG |
| | multiflorum | Contig | | GTTTGTACTGAACGTTCACTTTGCAAATGCGCCATAGTAATGATAGT |
| | | | | TCATGATTTTAGAGTACTTATTCCAATGTAAATTGTGGTTTAGCTACT |
| | | | | GCAATTACTGATTATATGTAGTCGATCCTTTAACCTCCTTGGCCATAC |
| | | | | GTATTCTGCAGATTGCACGAACTGGCAGGGTTGCGCTGAGCCGTGA |
| | | | | GTCAGGAGTTGATTCTAAGTACCTCCGTGGGTATTCGCTTCCTCTAT |
| | | | | AGCGTGTCAGTTGGCGAGACATCACGCTCTATCACTTTGCAGATTGC |
| | | | | CATTCTTCCGATCTCGGATCTCGTAAACTCTGAATATGGAATGGTGT |
| | | | | TTCATTTCTTTGGGAGCATACATCAAGAACACATTGGTCATAGTTAG |
| | | | | GATTTTTTTTTGTTCTTTTAGATACCAGAGAGAAATGGAGTTGGGAT |
| | | | | GTTTGAATCCTATGTGAATACAGGGTGGGAGAAGAAACTCATCCAT |
| | | | | AGTTTGCTTCTAGCGTCGCTGAGGTTTTATGATTCCCTCTTCGGGGT |
| | | | | GCTAAGCAGAATTAATTTTCTCCGACAGCCCATCACGTGGCTGCTAT |
| | | | | ATGTACCGCGCGGAGAGGTGTATACATCCAAAACCGTGTGCTTCCC |
| | | | | CACCGCTTACTCCTTCTCTCCGCCGCATTCTCTAACGTGATTTTGACG |
| | | | | CCTTCGATCGTGCTTGTGACCTAGAGATTCGGCACCCCATGATGCAC |
| | | | | GATGTGGTCGAGGAACTTCTCAATCGGAGTAGGGGTTGCTGGCCA |
| | | | | GGTTGCCGGCATCCCCGTGTGGCTGTGGAACCCTGGCCACCC |
| 1782 | Lolium | gDNA | 651 | GCCAGGAGAGGGTACAACATCGAGTCGTTGGCCGTCGGGCTGAAC |
| | multiflorum | Contig | | AAGGACAAGGCGCTGTTCACGATAGTGGTATCCGGAACAGAGAAA |
| | | | | ATACTGCAACAGGTTGTGGAGCAGCTCTACAAACTTGTCAACGTCA |
| | | | | TACAGGTCAGTATGTTCCTCCAATAAACCACTTGAATTACAAGATCA |
| | | | | TTGCTAACTGATACGTAGTAATGCGCCGTCAATTGAATCGTTCATTT |
| | | | | CATTTTTCGTGCTTGTTTCAGTCGTCAGATCCTCTGATATTCGTAGCT |
| | | | | TTGTATATTGATGAAACAGGTTGAGGATTTGTCAAAGGAGCCACAA |
| | | | | GTTAAAAAAGAGCTCATGCTGATAAAGCTGAACGTAGAGCCAGAG |
| | | | | CAGCGTCTTGAGGTAAATTTGTATGCGCGTAGGATGCTCGATTATAT |
| | | | | GCTTCAGTTGGAGTGGAGGTGAGGGTAGAATGCTGTTACTCTTTGC |
| | | | | TTATTTCTCCACTTGTACGTACATGCAGGTGATGGGTTTGGTTGACA |
| | | | | TTTTCAGAGCAAAAGTTGTTGATCTTTCAGACCGGACACTGACTATT |
| | | | | GAGGTAAAATCCATGGTGTGTTGTATCTTGCTGATCGCTTACATTGC |
| | | | | CACTTGTATGCTTTTATTAAAGCAAATTATGCGCTCTGTTTTTTGAGT |
| 1783 | Lolium | gDNA | 487 | ATGTAGTTCCAGGCTATTTTTCAATGAAACCATACTACTTCCCAGAG |
| | multiflorum | Contig | | TCTTGCTGTTGGCCGAGCTGAAAAGGAAGGCATTTCACGTATTACA |
| | | | | ACAGTTGTTCCCGGAACTGATGAATCCATCGAGAAGTTAGTTCAGC |
| | | | | AGCTTTACAAGCTTATTGATGTACTTAAGGTAAATGGCATGCAATTT |
| | | | | CTTAGGGCAATCATTTACGATATAAAATATGTTGTGTAATGCAAAAC |
| | | | | ATGTGATTGTCCCAGGTTGAGGACTTGACTCACTTACCTTTTGCCGA |
| | | | | AAGAGAACTGATGCTTATCAAGGTATCTGGGAACACCGCTGCTCGG |
| | | | | AGGGAGATACTAGATATCGGTCAAATCTTCCGGGCAGAATGTCTGG |
| | | | | ATCTTTCTGATCACACAGTTACGTTAATGGTGAGCTTCTGGCTTTATT |
| | | | | AGGTGTTGTTTGTTATTGTTGATTTAGCTTTACCAACTCTCAAGTGTG |
| | | | | CTATGGTGAGTATTAAAGC |
| 1784 | Lolium | gDNA | 475 | GTTTTGTTCACATACTTTGTCAATCCTTGTGAATGATTTCCCTGGAGT |
| | multiflorum | Contig | | TCTCAATGTTGTAACGGGTATCTTTTCCCGAAGGGGCTACAATATCC |
| | | | | AGGTTCGTTTTCCATTCGTAGATATCTTATGTTCCCTCAATTAGATCA |
| | | | | CTCCAGGTTAGTGTTGCGGTTCATTGTTTACAGACGTGCCCATTACT |
| | | | | TTGACAGAGTCTTGCTGTTGGTCCAGCTGAAAAAATAGGCACTTCTC |
| | | | | GCATCACTACTGTCGTTCCTGGGAGTGATGAATCTATCGCCAAGCTA |
| | | | | ATACATCAGCTTTACAAGCTCATTGACGTTTATGAGGTCAACTTATT |
| | | | | AATGTTGTGGTTTGCATGATTTTTTGTTGCCTATGTAAGGTTAGCTC |
| | | | | AAATTCGCCTTAGTTTTTCAGGTCCAAGATCTTACACATTTACCGTTT |
| | | | | ACTGCTAGAGAGTTAATGATCATCAAGGTCGCTGGGAACACCTCAG |
| | | | | CTT |
| 1785 | Lolium | gDNA | 373 | CGTGGCAGGTGATGGGGTTGGTTGACATTTTCAGAGCAAAAGTGG |
| | multiflorum | Contig | | TTGATCTTTCAGACCGGACACTGACTATTGAGGTAAAATCCATGGTG |
| | | | | TGTTGTATCTTGCTGCTCGCTTACATTATCACTTGTATGCTTTTATTA |
| | | | | AACCAAAGTATGCTCTCTGTTTTTTGAGTCATTCCAAAGGTAACTGG |
| | | | | AGATCCTGGAAAAATGGTAGCTGTACAGAGGAACCTGAGCAAATTT |
| | | | | GGGATCAAAGAAGTTACTAGAGGTGGTAAGGTTATAATTCTTGAGA |
| | | | | AAATATTAGAAACTTATTCGGCTTTTGTTCTTGGCTAGATCTAATGG |
| | | | | GCCTTATTAATGTGTAGATAGCTTTGCGGCGTGAGAAAATGGGACAA |
| 1786 | Lolium | gDNA | 343 | ATGTGCACTAAATTGCAATTTAAATCCCCATTTCGAAAATAACTGGC |
| | multiflorum | Contig | | ACACTTTTCTTTCTTCAGCCTACAGGGTTTTGTTCACATACTTTGTCA |
| | | | | ATCCTTGTGAATGATTTCCCTGGAGTTCTCAATGTTGTAACGGGTAT |
| | | | | CTTTTCCCGAAGGGGCTACAATATCCAGGTTTGTTTTCCATTTGTAG |
| | | | | ATATCTTATGTTCCCTCAATTAAATCACCCCGGGTTAGTGTTGCGGT |
| | | | | TCATTGTTAACAGACGTGTCCATTACTTTGACAGAGTCTTGCTGTTG |
| | | | | GTCCAGCTGAAAAAATAGGCACTTCTCGCATCACTACTGTTGTTCCT |
| | | | | GGGAGTGATGAAT |
| 1787 | Lolium | gDNA | 332 | CTTTACAAGCTCATCGACGTTTATGAGGTTAACTTATTAATGTTGTG |
| | multiflorum | Contig | | GTTTGCATTATTGTTGTTGCCTATGTAAGGTTAGCTCAAATTCGCCTT |
| | | | | GGTTTTTCAGGTCCAAGATCTTACACATTTACCGTTTACTGCTAGAG |
| | | | | AGTTAATGATCATCAAGGTCGCTGGGAACACCTCAGCTCGCAGGGC |
| | | | | TATCCTGGATATTGCTGAGGATGTTTTCGGGGCCAAAACGGTTGAT |
| | | | | GTATCTGACCACACAATAACTCTTCAGGTATTAATTACTTTTAACCTT |
| | | | | TTGTGCAGAACTATTGGATTTAGGACATCTATATGCTTTTCTTGGTA |
| | | | | GGA |
| 1788 | Lolium | gDNA | 285 | GGTCATGCTTGTAGCTAATGTGTTCAGAGCGAAAGTTGTTGATATTT |
| | multiflorum | Contig | | CCGAGAATTCTCTAACCCTAGAGGTAAAAACTTGGCTTCAAACAGT |
| | | | | ATTATAAGGACTAGGGTGTTCCGTGTCTATGCTGGATTAAATTTCTT |
| | | | | GATGCTGATCTGGCTAATGCTGCAGGTCACTGGAGATCCTGGAAAG |
| | | | | ATTGTTGCGGCACAAAGGAGCCTAAGCAAATTTGGGATCGAAGAA |
| | | | | ATTTGTAGAACGGGAAAAGTATTTTTCTGGACCATTTGCTACGTACA |
| | | | | TGCATGG |

We claim:

1. A method of plant control comprising: treating a plant with a composition comprising a polynucleotide and a transfer agent, wherein said polynucleotide is essentially identical or essentially complementary to an ALS gene sequence or fragment thereof, or to an RNA transcript of said ALS gene sequence or fragment thereof, wherein said ALS gene sequence is selected from the group consisting of SEQ ID NO:1-45 and 1692-1788 or a polynucleotide fragment thereof, whereby said plant growth or development or reproductive ability is reduced or said plant is more sensitive to an ALS inhibitor herbicide relative to a plant not treated with said composition.

2. The method as claimed in claim 1, wherein said transfer agent is an organosilicone surfactant composition or compound contained therein.

3. The method as claimed in claim 1, wherein said polynucleotide fragment is 18 contiguous, 19 contiguous nucleotides, 20 contiguous nucleotides or at least 21 contiguous nucleotides in length and at least 85 percent identical to an ALS gene sequence selected from the group consisting of SEQ ID NO:1-45 and 1692-1788.

4. The method as claimed in claim 3, wherein said polynucleotide fragment is selected from the group consisting of sense or anti-sense ssDNA or ssRNA, dsRNA, or dsDNA, or dsDNA/RNA hybrids.

5. The method as claimed in claim 1, wherein said plant is selected from the group consisting of Amaranthus palmeri, Amaranthus rudis, Amaranthus chlorostachys, Amaranthus graecizans, Amaranthus hybridus, Amaranthus lividus, Amaranthus spinosus, Amaranthus thunbergii, Amaranthus viridis, Ambrosia trifida, Kochia scoparia, Abutilon theophrasti, Chenopodium album, Commelina diffusa, Conyza candensis Digitaria sanguinalis Euphorbia heterophylla, and Lolium multiflorum.

6. The method as claimed in claim 1, wherein said composition further comprises said ALS inhibitor herbicide and external application to a plant with said composition.

7. The method as claimed in claim 6, wherein said composition further comprises one or more herbicides different from said ALS inhibitor herbicide.

8. The method as claimed in claim 3, wherein said composition comprises any combination of two or more of said polynucleotide fragments and external application to a plant with said composition.

9. A composition comprising a polynucleotide and a transfer agent, wherein said polynucleotide is essentially identical or essentially complementary to an ALS gene sequence or fragment thereof, or to an RNA transcript of said ALS gene sequence or fragment thereof, wherein said ALS gene sequence is selected from the group consisting of SEQ ID NO:1-45 and 1692-1788 or a polynucleotide fragment thereof, and whereby a plant treated with said composition has its growth or development or reproductive ability regulated, suppressed or delayed or said plant is more sensitive to an ALS inhibitor herbicide as a result of said polynucleotide containing composition relative to a plant not treated with said composition.

10. The composition of claim 9, wherein said transfer agent is an organosilicone composition.

11. The composition of claim 9, wherein said polynucleotide fragment is 18 contiguous, 19 contiguous nucleotides, 20 contiguous nucleotides or at least 21 contiguous nucleotides in length and at least 85 percent identical to an ALS gene sequence selected from the group consisting of SEQ ID NO:1-45 and 1692-1788.

12. The composition of claim 9, wherein said polynucleotide is selected from the group consisting of SEQ ID NO: 46-1363 and 1789-4166.

13. The composition of claim 9, wherein said polynucleotide is selected from the group consisting of SEQ ID NO: 1364-1691 and 4167-4201.

14. The composition of claim 9, further comprising an ALS inhibitor herbicide.

15. The composition of claim 14, wherein said ALS inhibitor molecule is selected from the group consisting of amidosulfuron, azimsulfuron, bensulfuron-methyl, chlorimuron-ethyl, chlorsulfuron, cinosulfuron, cyclosulfamuron, ethametsulfuron-methyl, ethoxysulfuron, flazasulfuron, flupyrsulfuron-methyl-Na, foramsulfuron, halosulfuron-methyl, imazosulfuron, iodosulfuron, metsulfuron-methyl, nicosulfuron, oxasulfuron, primisulfuron-methyl, prosulfuron, pyrazosulfuron-ethyl, rimsulfuron, sulfometuron-methyl, sulfosulfuron, thifensulfuron-methyl, triasulfuron, tribenuron-methyl, trifloxysulfuron, triflusulfuron-methyl, tritosulfuron, imazapic, imazamethabenz-methyl, imazamox, imazapyr, imazaquin, imazethapyr, cloransulam-methyl, diclosulam, florasulam, flumetsulam, metosulam, bispyribac-Na, pyribenzoxim, pyriftalid, pyrithiobac-Na, pyriminobac-methyl, flucarbazone-Na, and procarbazone-Na.

16. The composition of claim 14, further comprising a co-herbicide.

17. A method of reducing expression of an ALS gene in a plant comprising: external application to a plant of a composition comprising a polynucleotide and a transfer agent, wherein said polynucleotide is essentially identical or essentially complementary to an ALS gene sequence or fragment thereof, or to the RNA transcript of said ALS gene sequence or fragment thereof, wherein said ALS gene sequence is selected from the group consisting of SEQ ID NO:1-45 and 1692-1788 or a polynucleotide fragment thereof, whereby said expression of said ALS gene is reduced relative to a plant in which the composition was not applied.

18. The method as claimed in claim 17, wherein said transfer agent is an organosilicone compound.

19. The method as claimed in claim 17, wherein said polynucleotide fragment is 19 contiguous nucleotides, 20 contiguous nucleotides or at least 21 contiguous nucleotides in length and at least 85 percent identical to an ALS gene sequence selected from the group consisting of SEQ ID NO:1-45 and 1692-1788.

20. The method as claimed in 17, wherein said polynucleotide molecule is selected from the group consisting of sense or anti-sense ssDNA or ssRNA, dsRNA, or dsDNA, or dsDNA/RNA hybrids.

21. A microbial expression cassette comprising a polynucleotide fragment of 18 contiguous, 19 contiguous nucleotides, 20 contiguous nucleotides or at least 21 contiguous nucleotides in length and at least 85 percent identical to an ALS gene sequence selected from the group consisting of SEQ ID NO:1-45 and 1692-1788.

22. A method of making a polynucleotide comprising a) transforming the microbial expression cassette of claim 21 into a microbe; b) growing said microbe; c) harvesting a polynucleotide from said microbe, wherein said polynucleotide is 18 contiguous, 19 contiguous nucleotides, 20 contiguous nucleotides or at least 21 contiguous nucleotides in length and at least 85 percent identical to an ALS gene sequence selected from the group consisting of SEQ ID NO:1-45 and 1692-1788.

23. A method of identifying polynucleotides useful in modulating ALS gene expression when externally treating a plant comprising: a) providing a plurality of polynucleotides that comprise a region essentially identical or essentially complementary to a polynucleotide fragment of 18 contiguous, 19 contiguous nucleotides, 20 contiguous nucleotides or at least 21 contiguous nucleotides in length and at least 85 percent identical to an ALS gene sequence selected from the group consisting of SEQ ID NO:1-45 and 1692-1788; b) externally treating said plant with one or more of said polynucleotides and a transfer agent; c) analyzing said plant or extract for modulation of ALS gene expression, and whereby a plant treated with said composition has its growth or development or reproductive ability regulated, suppressed or delayed or said plant is more sensitive to an EPSPS inhibitor herbicide as a result of said polynucleotide containing composition relative to a plant not treated with said composition.

24. The method as claimed in 23, wherein said plant is selected from the group consisting of Amaranthus palmeri, Amaranthus rudis, Amaranthus chlorostachys, Amaranthus graecizans, Amaranthus hybridus, Amaranthus lividus, Amaranthus spinosus, Amaranthus thunbergii, Amaranthus viridis, Ambrosia trifida, Kochia scoparia, Abutilon theophrasti, Chenopodium album, Commelina diffusa, Conyza candensis Digitaria sanguinalis Euphorbia heterophylla, and Lolium multiflorum.

25. The method as claimed in 23, wherein said ALS gene expression is reduced relative to a plant not treated with said polynucleotide fragment and a transfer agent.

26. The method as claimed in 23, wherein said transfer agent is an organosilicone compound.

27. An agricultural chemical composition comprising an admixture of a polynucleotide and a glyphosate herbicide and a co-herbicide, wherein said polynucleotide is essentially identical or essentially complementary to a portion of an ALS gene sequence, or to a portion of an RNA transcript of said ALS gene sequence, wherein said ALS gene sequence is selected from the group consisting of SEQ ID NO: 1-45 and 1692-1788 or a polynucleotide fragment thereof, and whereby a plant treated with said composition has its growth or development or reproductive ability regulated, suppressed or delayed or said plant is more sensitive to an ALS inhibitor herbicide as a result of said polynucleotide containing composition relative to a plant not treated with said composition.

28. The agricultural chemical composition of claim 27, wherein said co-herbicide is selected from the group consisting of amide herbicides, arsenical herbicides, benzothiazole herbicides, benzoylcyclohexanedione herbicides, benzofuranyl alkylsulfonate herbicides, carbamate herbicides, cyclohexene oxime herbicides, cyclopropylisoxazole herbicides, dicarboximide herbicides, dinitroaniline herbicides, dinitrophenol herbicides, diphenyl ether herbicides, dithiocarbamate herbicides, halogenated aliphatic herbicides, imidazolinone herbicides, inorganic herbicides, nitrile herbicides, organophosphorus herbicides, oxadiazolone herbicides, oxazole herbicides, phenoxy herbicides, phenylenediamine herbicides, pyrazole herbicides, pyridazine herbicides, pyridazinone herbicides, pyridine herbicides, pyrimidinediamine herbicides, pyrimidinyloxybenzylamine herbicides, quaternary ammonium herbicides, thiocarbamate herbicides, thiocarbonate herbicides, thiourea herbicides, triazine herbicides, triazinone herbicides, triazole herbicides, triazolone herbicides, triazolopyrimidine herbicides, uracil herbicides, and urea herbicides.

29. An agricultural chemical composition comprising an admixture of a polynucleotide and a glyphosate herbicide and a pesticide, wherein said polynucleotide is essentially identical or essentially complementary to a portion of an ALS gene sequence or a fragment thereof, or to a portion of an RNA transcript of said ALS gene sequence or a fragment thereof, wherein said ALS gene sequence is selected from the group consisting of SEQ ID NO:1-45 and 1692-1788 or a polynucleotide fragment thereof, and whereby a plant treated with said composition has its growth or development or reproductive ability regulated, suppressed or delayed or said plant is more sensitive to an ALS inhibitor herbicide as a result of said polynucleotide containing composition relative to a plant not treated with said composition.

30. The agricultural chemical composition of claim 29, wherein said pesticide is selected from the group consisting of insecticides, fungicides, nematocides, bactericides, acaricides, growth regulators, chemosterilants, semiochemicals, repellents, attractants, pheromones, feeding stimulants, and biopesticides.

31. A polynucleotide molecule applied to the surface of a plant that enhances said plant sensitivity to a glyphosate containing herbicide composition, wherein said polynucleotide comprises a homologous or complementary polynucleotide having at least 85 percent identity to a polynucleotide selected from the group consisting of SEQ ID NO:4202 and 4218-4247.
